(19) United States
(12) Patent Application Publication
(10) Pub. No.: US 2025/0161474 A1
(43) Pub. Date: May 22, 2025

(54) ANTIBODY-DRUG CONJUGATES TARGETING EGFR/C-MET AND PREPARATION METHODS AND USES THEREOF

(71) Applicant: Sichuan Kelun-Biotech Biopharmaceutical Co., Ltd., Chengdu (CN)

(72) Inventors: Yong Zheng, Chengdu (CN); Qiang Tian, Chengdu (CN); Dongmei Lei, Chengdu (CN); Yanshu Di, Chengdu (CN); Yuehua Zhou, Chengdu (CN); Ruibin Hu, Chengdu (CN); Hu Long, Chengdu (CN); Yuzhi Pu, Chengdu (CN); Xiaoxi Yuan, Chengdu (CN); Hongmei Song, Chengdu (CN); Miao Tan, Chengdu (CN); Xiangyang Tan, Chengdu (CN); Junyou Ge, Chengdu (CN)

(21) Appl. No.: 18/949,295

(22) Filed: Nov. 15, 2024

(30) Foreign Application Priority Data
Nov. 15, 2023 (CN) 202311531642.X
Nov. 24, 2023 (CN) 202311596146.2
Nov. 7, 2024 (CN) 202411591580.6

(51) Int. Cl. A61K 47/68; A61K 45/06; A61P 35/00; C07K 16/28

(52) U.S. Cl. CPC A61K 47/68037; A61K 45/06; A61K 47/6845; A61K 47/6889; A61P 35/00; C07K 16/2863

(57) ABSTRACT

Antibody-drug conjugates (ADC) useful for preventing, treating, and/or acting as an adjuvant in treating c-MET and/or EGFR-related diseases are disclosed. The ADCs comprise a bispecific antibody or antigen-binding fragment thereof comprising an arm specific for binding to EGFR and an arm specific for binding to c-MET conjugated to a cytotoxic drug.

### REFERENCE TO RELATED APPLICATIONS

This application claims the priority of and benefit to Chinese Patent Application Serial No. 2023-11531642.X, filed Nov. 15, 2023, Chinese Patent Application Serial No. 2023-11596146.2, filed Nov. 24, 2023, and Chinese Patent Application Serial No. 2024-11591580.6, filed Nov. 7, 2024, the entire disclosures of each of which are incorporated herein by reference.

### SEQUENCE LISTING

The content of the electronically submitted Sequence Listing XML (Name: 214279_seglist.xml; Size: 88,294 bytes; Created: Nov. 14, 2024) is herein incorporated by reference in its entirety.

### FIELD OF THE INVENTION

The present disclosure relates to targeted therapy, and more specifically, to antibody-drug conjugates targeting EGFR and c-MET and methods of preparation and use thereof.

### DESCRIPTION OF RELATED ART

EGFR (epidermal growth factor receptor, referred to as EGFR, ErbB-1 or HER1) is a member of the epidermal growth factor receptor (HER) family. This family includes HER1 (erbB1, EGFR), HER2 (erbB2, NEU), HER3 (erbB3), and HER4 (erbB4). The HER family plays important regulatory roles in cellular physiological processes. EGFR is a type I transmembrane glycoprotein composed of 1210 amino acids, with a molecular weight of about 170 kDa. Its main structure includes a ligand-binding extracellular domain, a hydrophobic transmembrane region, and an intracellular region comprising tyrosine kinase. EGFR is highly expressed in various tumors such as colorectal cancer, head and neck cancer, and non-small cell lung cancer. It is also expressed to a certain extent in normal epithelial tissues such as skin and lungs. EGFR, which is highly expressed in tumor tissues, undergoes homologous or heterologous dimerization through the binding of ligands such as EGF or TGFα. Dimerization leads to the activation of tyrosine kinases and protein phosphorylation in tumor cells, mediating gene transcription and cell cycle. The activation of various cell signaling pathways plays an important role in promoting the survival, proliferation and migration of tumor cells and tumor angiogenesis.

Tyrosine protein kinase Met (c-MET), also known as hepatocyte growth factor receptor (HGFR), is a heterodimeric transmembrane tyrosine kinase receptor encoded by the Met proto-oncogene. c-MET is a type I transmembrane glycoprotein composed of 1390 amino acids, with a molecular weight of about 190 kDa. It eventually forms two polypeptide chains connected by disulfide bonds through shearing, namely a chain (50 kDa) and R chain (140 kDa). MET protein, from outside the membrane to inside the cell, can be divided into SEMA domain (Semaphorin domain, SEMA), PSI domain (Plexin-semaphorin-integrin domain, PSI), four immunoglobulin-like repeat domains (Immunoglobulin-plexins-transcription domain, IPT), a transmembrane region (Transmembrane region), a juxtamembrane region (JM), tyrosine kinase domain (TK) and a carboxyl terminal region (Carboxyl terminal region, CT). The natural ligand of the c-MET receptor is hepatocyte growth factor (HGF), an inactive protein that is converted to its active form by proteolytic cleavage. Abnormal regulation of c-MET has been reported in a variety of cancers, including colorectal cancer, non-small cell lung cancer, gastric cancer, and breast cancer. High activation of c-MET and its downstream signaling pathways has been shown to trigger hyperproliferation, tumor invasion, and angiogenesis, and is associated with poor survival.

Nearly 60% of tumors resistant to EGFR tyrosine kinase inhibitors (TKIs) have increased c-MET expression, amplified c-MET, or increased c-MET ligand HGF (Turke et al., Cancer Cell, 17: 77-88, 2010), indicating that the tumor c-MET signaling pathway will compensate for the EGFR signaling pathway. Both EGFR and c-MET signal are transduced through the same survival and anti-apoptotic pathways (ERK and AKT). Targeting EGFR and c-MET at the same time is expected to inhibit the two signaling pathways of EGFR and c-MET at the same time, avoiding the occurrence and development of tumors caused by the activation of the c-MET signaling pathway after EGFR inhibition, thereby improving the overall efficacy and safety.

Anti-c-MET and anti-EGFR antibodies are known in the art, for example, Telisotuzumab vedotin (Teliso-v or ABBV399), which is disclosed in U.S. patent Ser. No. 10/603,389, is an anti-c-MET antibody ABT-700 conjugated to vc-MMAE (vedotin), and MRG003 (ADC-3 or MYK-3), which is disclosed in WO2023040941, is an anti-EGFR antibody BA03 conjugated to vedotin.

Several EGFR/c-Met bispecific antibodies have been disclosed in the art. Bispecific antibody BSAB01 disclosed in WO2010115551 comprises the EGFR binding VH/VL pair (Fab) of cetuximab and the c-Met binding VH/VL pair (Fab) of onartuzumab, which is currently in Phase III trials. Amivantamab, a bispecific antibody marketed under the tradename RYBREVANT®, is disclosed in U.S. Pat. No. 9,593,164. Amivantamab comprises an Anti-EGFR Fab comprising the VH and VL of Zalutumumab (See, U.S. Pat. Nos. 7,247,301 and 7,595,378) and an anti-cIET Fab comprising the VH and VL of antibody 069 disclosed in U.S. Pat. No. 9,068,011. EGFR/c-Met bispecific antibodies conjugated to cytotoxins have been disclosed in Sellman et al. (J Biol Chem. 291(48): 25106-25119 (2016)), which discloses an EGFR/c-Met bispecific antibody conjugated to vedotin, and in WO2023083846, which discloses AZD9592, an EGFR/c-Met bispecific antibody conjugated to a camptothecin derivative. There is a need in the art for antibody-drug conjugates (ADC) that bind to both EGFR and c-MET and comprise cytotoxic drugs.

### SUMMARY OF THE INVENTION

The present invention provides antibody-drug conjugates (ADC) useful for preventing, treating, and/or acting as an adjuvant in treating c-MET and/or EGFR-related diseases. The ADCs of the present invention comprise a bispecific antibody comprising an arm specific for binding to EGFR and an arm specific for binding to c-MET conjugated to a cytotoxic drug. In particular embodiments, the bispecific antibody comprises an scFv that binds EGFR and a Fab that binds c-MET. In other embodiments, the bispecific antibody comprises a Fab that binds EGFR and an scFv that binds c-MET or comprises an scFv that binds EGFR and an scFv that binds c-MET. In further embodiments, the anti-EGFR Fab and/or the anti-c-MET Fab comprises a CrossMab format selected from CrossMab^{Fab}, CrossMab^{VH-VL}, and CrossMab^{CH1-CL}. The ADCs and medicaments thereof are useful for preventing, treating, and/or acting as an adjuvant in treating diseases (e.g., cancer) associated with c-MET and/or EGFR.

Exemplary ADCs comprising bispecific antibodies 07B, 10B, 38B, 41B, 49B, 55B or 56B disclosed herein conjugated to a camptothecin derivative display affinity for EGFR and c-MET and killing activity in vivo and in vitro against EGFR and/or c-MET positive tumor cells, such as gastric cancer, non-small cell lung cancer (e.g., lung adenocarcinoma), epidermal squamous cell carcinoma, and the like.

The present invention provides an antibody-drug conjugate comprising a structure shown in formula:

Ab-[M-L-E-D]ₓ
wherein:
Ab is a bispecific antibody or bispecific antigen-binding fragment thereof comprising a first antigen-binding domain specifically binding to c-MET and a second antigen-binding domain specifically binding to EGFR, wherein the first antigen-binding domain comprises a first light chain variable region (VL) and a first heavy chain variable region (VH), and the first VL and the first VH collectively form a domain capable of specifically binding to c-MET; and the second antigen-binding domain comprises a second VL and a second VH, and the second VL and the second VH together form a domain capable of specifically binding to EGFR;
M is a linker site connected to the bispecific antibody or bispecific antigen-binding fragment thereof,
L is a structural fragment connecting the linker sites M and E;
E is a structural fragment connecting L and D;
D is a cytotoxic drug or residue thereof, and
x is selected as any integer from 1 to 10.

In a further embodiment of the antibody-drug conjugate, the first antigen-binding domain and the second antigen-binding domain are each independently selected from scFv, Fab, and scFab.

In a further embodiment of the antibody-drug conjugate, the bispecific antibody or bispecific antigen-binding fragment thereof further comprises an Fc domain comprising a first Fc domain monomer and a second Fc domain monomer, and wherein the first and second Fc domain monomers comprise one or more modifications that promote heterodimerization of the Fc domain monomers.

In a further embodiment of the antibody-drug conjugate, the Fc domain comprises a first Fc domain monomer comprising modifications that form a knob structure and a second Fc domain monomer comprising modifications that form a hole structure, wherein the hole structure can be paired with the knob structure to form a heterodimeric Fc domain.

In a further embodiment of the antibody-drug conjugate, the first Fc domain monomer comprises the amino acid sequence as shown in SEQ ID NO: 49 or 51, and the second Fc domain monomer comprises the amino acid sequence as shown in SEQ ID NO: 50 or 52.

In a further embodiment of the antibody-drug conjugate, the first antigen-binding domain and the second antigen-binding domain are each linked to one of the first and second Fc domain monomers of the Fc domain.

In a further embodiment of the antibody-drug conjugate, the first antigen-binding domain is linked to the first Fc domain monomer, and the second antigen-binding domain is linked to the second Fc domain monomer; or the first antigen-binding domain is linked to the second Fc domain monomer, and the second antigen-binding domain is linked to the first Fc domain monomer.

In a further embodiment of the antibody-drug conjugate, the first VL comprises the complementarity determining region (CDR)-L1, CDR-L2, and CDR-L3 amino acid sequences of a VL amino acid sequence set forth in SEQ ID NO: 17 or SEQ ID NO: 59; and/or the first VH comprises the CDR-H1, CDR-H2, and CDR-H3 amino acid sequences of a VH amino acid sequence set forth in SEQ ID NO: 18 or SEQ ID NO: 60.

In a further embodiment of the antibody-drug conjugate, the first VL comprises:
(i) a CDR-L1 comprising the amino acid sequence as shown in SEQ ID NO: 34, a CDR-L2 comprising the amino acid sequence as shown in SEQ ID NO: 36, and a CDR-L3 comprising the amino acid sequence as shown in SEQ ID NO: 38; or
(ii) a CDR-L1 comprising the amino acid sequence as shown in SEQ ID NO: 35, a CDR-L2 comprising the amino acid sequence as shown in SEQ ID NO: 37, and a CDR-L3 comprising the amino acid sequence as shown in SEQ ID NO: 38; and/or
the first VH comprises:
(i) a CDR-H1 comprising the amino acid sequence as shown in SEQ ID NO: 39, a CDR-H2 comprising the amino acid sequence as shown in SEQ ID NO: 43, and a CDR-H3 comprising the amino acid sequence as shown in SEQ ID NO: 47;
(ii) a CDR-H1 comprising the amino acid sequence as shown in SEQ ID NO: 40, a CDR-H2 comprising the amino acid sequence as shown in SEQ ID NO: 44, and a CDR-H3 comprising the amino acid sequence as shown in SEQ ID NO: 47;
(iii) a CDR-H1 comprising the amino acid sequence as shown in SEQ ID NO: 42, a CDR-H2 comprising the amino acid sequence as shown in SEQ ID NO: 46, and a CDR-H3 comprising the amino acid sequence as shown in SEQ ID NO: 47; or
(iv) a CDR-H1 comprising the amino acid sequence as shown in SEQ ID NO: 41, a CDR-H2 comprising the amino acid sequence as shown in SEQ ID NO: 45, and a CDR-H3 comprising the amino acid sequence as shown in SEQ ID NO: 48.

In a further embodiment of the antibody-drug conjugate, the first VL comprises the amino acid sequence as shown in SEQ ID NO: 17 or 59 and/or the first VH comprises the amino acid sequence as shown in SEQ ID NO: 18 or 60; or the first VL comprises the amino acid sequence as shown in SEQ ID NO: 17, the first VH comprises the amino acid sequence as shown in SEQ ID NO: 18; or the first VL comprises the amino acid sequence as shown in SEQ ID NO: 59, the first VH comprises the amino acid sequence as shown in SEQ ID NO: 60.

In a further embodiment of the antibody-drug conjugate, the second VL comprises the CDR-L1, CDR-L2, and CDR-L3 amino acid sequences of a VL amino acid sequence set forth in SEQ ID NO: 15; and/or the second VH comprises the CDR-H1, CDR-H2, and CDR-H3 amino acid sequences of a VH amino acid sequence set forth in SEQ ID NO: 16.

In a further embodiment of the antibody-drug conjugate, the second VL comprises:
(i) a CDR-L1 comprising the amino acid sequence as shown in SEQ ID NO: 19, a CDR-L2 comprising the amino acid sequence as shown in SEQ ID NO: 21, and a CDR-L3 comprising the amino acid sequence as shown in SEQ ID NO: 23; or
(ii) a CDR-L1 comprising the amino acid sequence as shown in SEQ ID NO: 20, a CDR-L2 comprising the amino acid sequence as shown in SEQ ID NO: 22, and a CDR-L3 comprising the amino acid sequence as shown in SEQ ID NO: 23; and/or
the second VH comprises:
(i) a CDR-H1 comprising the amino acid sequence as shown in SEQ ID NO: 24, a CDR-H2 comprising the amino acid sequence as shown in SEQ ID NO: 28, and a CDR-H3 comprising the amino acid sequence as shown in SEQ ID NO: 32;
(ii) a CDR-H1 comprising the amino acid sequence as shown in SEQ ID NO: 25, a CDR-H2 comprising the amino acid sequence as shown in SEQ ID NO: 29, and a CDR-H3 comprising the amino acid sequence as shown in SEQ ID NO: 32;
(iii) a CDR-H1 comprising the amino acid sequence as shown in SEQ ID NO: 27, a CDR-H2 comprising the amino acid sequence as shown in SEQ ID NO: 31, and a CDR-H3 comprising the amino acid sequence as shown in SEQ ID NO: 32; or
(iv) a CDR-H1 comprising the amino acid sequence as shown in SEQ ID NO: 26, a CDR-H2 comprising the amino acid sequence as shown in SEQ ID NO: 30, and a CDR-H3 comprising the amino acid sequence as shown in SEQ ID NO: 33.

In a further embodiment of the antibody-drug conjugate, the second VL comprises the amino acid sequence as shown in SEQ ID NO: 15, and/or the second VH comprises the amino acid sequence as shown in SEQ ID NO: 16.

In a further embodiment of the antibody-drug conjugate, the first antigen-binding domain is a Fab, and the second antigen-binding domain is an scFv.

In a further embodiment of the antibody-drug conjugate, the bispecific antibody comprises a polypeptide chain I-A, a polypeptide chain I-B and a polypeptide chain I-C; wherein the polypeptide chain I-A comprises the first VL and a light chain constant region; the polypeptide chain I-B comprises: the VH, a heavy chain CH1 region and the first Fc domain monomer (or the second Fc domain monomer); the polypeptide chain I-C comprises: the second VL, the second VH and the second Fc domain monomer (or the first Fc domain monomer).

In a further embodiment of the antibody-drug conjugate, the polypeptide chain I-A comprises from N-terminus to C-terminus the first VL and the light chain constant region; the polypeptide chain I-B comprises from N-terminus to C-terminus the first VH, the heavy chain CH1 region and the first Fc domain monomer (or the second Fc domain monomer); and/or the polypeptide chain I-C comprises from N-terminus to C-terminus (i) the second VL, the second VH and the second Fc domain monomer (or the first Fc domain monomer), or (ii) the second VH, the second VL and the second Fc domain monomer (or the first Fc domain monomer).

In a further embodiment of the antibody-drug conjugate, the adjacent domains of the polypeptide chain I-A are connected optionally with or without a linker, and the adjacent domains of the polypeptide chain I-B are connected optionally with or without a linker, and/or the adjacent domains of the polypeptide chain I-C are connected optionally with or without a linker.

In a further embodiment of the antibody-drug conjugate, the linkers are each independently the same peptide linker or different peptide linkers (e.g., rigid peptide linkers or flexible peptide linkers); or the peptide linkers are each independently selected from the group consisting of peptide linkers comprising one or more glycines (G) and/or serines (S), for example, having the structure shown as (GGGGS)ₙ, wherein n is selected from 1, 2, 3, 4, 5, 6, 7, 8, 9 or 10 (SEQ ID NOs: 55-58 or 65-70); or the peptide linkers each independently comprise the amino acid sequence shown in SEQ ID NOs: 55-58, 61, or 65-70.

In a further embodiment of the antibody-drug conjugate, the light chain constant region comprises the amino acid sequence as shown in SEQ ID NO: 53 or SEQ ID NO: 63, and/or the heavy chain CH1 region comprises the amino acid sequence as shown in SEQ ID NO: 54.

In a further embodiment of the antibody-drug conjugate, the polypeptide chain I-A comprises the amino acid sequence as shown in SEQ ID NO: 1, the polypeptide chain I-B comprises the amino acid sequence as shown in SEQ ID NO: 2 or 9, and/or the polypeptide chain I-C comprises the amino acid sequence as shown in SEQ ID NO: ID NO: 3 or 10

In a further embodiment of the antibody-drug conjugate, the first antigen-binding domain is an scFv, and the second antigen-binding domain is a Fab.

In a further embodiment of the antibody-drug conjugate, the bispecific antibody comprises a polypeptide chain II-A, a polypeptide chain II-B and a polypeptide chain II-C; wherein the polypeptide chain II-A comprises the second VL and a light chain constant region; the polypeptide chain II-B comprises the second VH, a heavy chain CH1 region and the first Fc domain monomer (or the second Fc domain monomer), the polypeptide chain II-C comprises: the first VL, the first VH and the second Fc domain monomer (or the first Fc domain monomer).

In a further embodiment of the antibody-drug conjugate, the polypeptide chain II-A comprises from N-terminus to C-terminus the second VL and the light chain constant region; and the polypeptide chain II-B comprises from N-terminus to C-terminus the second VH, the heavy chain CH1 region and the first Fc domain monomer (or the second Fc domain monomer); and/or the polypeptide chain II-C comprises from N-terminus to C-terminus (i) the first VL, the first VH and the second Fc domain monomer (or the first Fc domain monomer), or (ii) the first VH, the first VL and the second Fc domain monomer (or the first Fc domain monomer).

In a further embodiment of the antibody-drug conjugate, the adjacent domains of the polypeptide chain II-A are connected optionally with or without a linker; the adjacent domains of the polypeptide chain II-B are connected optionally with or without a linker, and/or the adjacent domains of the polypeptide chain II-C are connected optionally with or without a linker.

In a further embodiment of the antibody-drug conjugate, the linkers are each independently the same peptide linker or different peptide linkers (e.g., rigid peptide linkers or flexible peptide linkers); or the peptide linkers are each independently selected from peptide linkers comprising one or more glycines (G) and/or serines (S), for example, having the structure shown in (GGGGS)ₙ, wherein n is selected from 1, 2, 3, 4, 5, 6, 7, 8, 9 or 10 (SEQ ID NOs: 55-58 or 65-70); or the peptide linkers each independently comprise the amino acid sequence shown in SEQ ID NOs: 55-58, 61, or 65-70.

In a further embodiment of the antibody-drug conjugate, the light chain constant region comprises the amino acid sequence as shown in SEQ ID NO: 53 or SEQ ID NO: 63, and/or the heavy chain CH1 region comprises the amino acid sequence as shown in SEQ ID NO: 54.

In a further embodiment of the antibody-drug conjugate, the polypeptide chain II-A comprises the amino acid sequence as shown in SEQ ID NO: 4, the polypeptide chain II-B comprises the amino acid sequence as shown in SEQ ID NO: 5 or 7, and/or the polypeptide chain II-C comprises the amino acid sequence as shown in SEQ ID NO: 6 or 8.

In a further embodiment of the antibody-drug conjugate, the first antigen-binding domain is a Fab, and the second antigen-binding domain is an scFab.

In a further embodiment of the antibody-drug conjugate, the bispecific antibody comprises a polypeptide chain III-A, a polypeptide chain III-B and a polypeptide chain III-C; wherein the polypeptide chain III-A comprises the first VL and a light chain constant region; the polypeptide chain III-B comprises: the first VH, a heavy chain CH1 region and the first Fc domain monomer (or the second Fc domain monomer), the polypeptide chain III-C comprises: the second VL, a light chain constant region, the second VH, a heavy chain CH1 region and the second Fc domain monomer (or the first Fc domain monomer).

In a further embodiment of the antibody-drug conjugate, the polypeptide chain III-A comprises from N-terminus to C-terminus the first VL and the light chain constant region; the polypeptide chain III-B comprises from N-terminus to C-terminus the first VH, the heavy chain CH1 region and the first Fc domain monomer (or the second Fc domain monomer); and/or the polypeptide chain III-C comprises from N-terminus to C-terminus (i) the second VL, the light chain constant region, the second VH, the heavy chain CH1 region and the second Fc domain monomer (or the first Fc domain monomer), or (ii) the second VH, the heavy chain CH1 region, the second VL, the light chain constant region, and the second Fc domain monomer (or the first Fc domain monomer).

In a further embodiment of the antibody-drug conjugate, the adjacent domains of the polypeptide chain III-A are connected optionally with or without a linker; the adjacent domains of the polypeptide chain III-B are connected optionally with or without a linker; and/or the adjacent domains of the polypeptide chain III-C are connected optionally with or without a linker.

In a further embodiment of the antibody-drug conjugate, the linkers are each independently the same peptide linker or different peptide linkers (e.g., rigid peptide linkers or flexible peptide linkers); or the peptide linkers are each independently selected from peptide linkers comprising one or more glycines (G) and/or serines (S), for example, having the structure shown in (GGGGS)ₙ, wherein n is selected from 1, 2, 3, 4, 5, 6, 7, 8, 9 or 10 (SEQ ID NOs: 55-58 or 65-70); or the peptide linkers each independently comprise the amino acid sequence shown in SEQ ID NOs: 55-58, 61, or 65-70.

In a further embodiment of the antibody-drug conjugate, the light chain constant region comprises the amino acid sequence as shown in SEQ ID NO: 53 or SEQ ID NO: 63, and/or the CH1 region of the heavy chain comprises the amino acid sequence as shown in SEQ ID NO: 54.

In a further embodiment of the antibody-drug conjugate, the first antigen-binding domain is an scFab, and the second antigen-binding domain is a Fab.

In a further embodiment of the antibody-drug conjugate, the bispecific antibody comprises a polypeptide chain IV-A, a polypeptide chain IV-B and a polypeptide chain IV-C; wherein the polypeptide chain IV-A comprises the second VL and a light chain constant region; the polypeptide chain IV-B comprises: the second VH, a heavy chain CH1 region and the first Fc domain monomer (or the second Fc domain monomer); the polypeptide chain IV-C comprises: the first VL, a light chain constant region, the first VH, a heavy chain CH1 region and the second Fc domain monomer (or the first Fc domain monomer).

In a further embodiment of the antibody-drug conjugate, the polypeptide chain IV-A comprises from N-terminus to C-terminus the second VL and the light chain constant region, and the polypeptide chain IV-B comprises from N-terminus to C-terminus the second VH, the CH1 region of the heavy chain and the first Fc domain monomer (or the second Fc domain monomer); and/or the polypeptide chain IV-C comprises from N-terminus to C-terminus (i) the first VL, the light chain constant region, the first VH, the heavy chain CH1 region and the second Fc domain monomer (or the first Fc domain monomer); or (ii) the first VH, the heavy chain CH1 region, the first VL, the light chain constant region, and the second Fc domain monomer (or the first Fc domain monomer).

In a further embodiment of the antibody-drug conjugate, the adjacent domains of the polypeptide chain IV-A are connected optionally with or without a linker, and the adjacent domains of the polypeptide chain IV-B are connected optionally with or without a linker; and/or the adjacent domains of the polypeptide chain IV-C are connected optionally with or without a linker.

In a further embodiment of the antibody-drug conjugate, the linkers are each independently the same peptide linker or different peptide linkers (e.g., rigid peptide linkers or flexible peptide linkers); or the peptide linkers are each independently selected from peptide linkers comprising one or more glycines (G) and/or serines (S), for example, having the structure shown in (GGGGS)ₙ, wherein n is selected from 1, 2, 3, 4, 5, 6, 7, 8, 9 or 10 (SEQ ID NOs: 55-58 or 65-70); or the peptide linkers each independently comprise the amino acid sequence shown in SEQ ID NOs: 55-58, 61, or 65-70.

In a further embodiment of the antibody-drug conjugate, the light chain constant region comprises the amino acid sequence as shown in SEQ ID NO: 53 or SEQ ID NO: 63, and/or the heavy chain CH1 region comprises the amino acid sequence as shown in SEQ ID NO: 54.

In a further embodiment of the antibody-drug conjugate, the polypeptide chain IV-A comprises the amino acid sequence as shown in SEQ ID NO: 4, the polypeptide chain IV-B comprises the amino acid sequence as shown in SEQ ID NO: 7, and/or the polypeptide chain IV-C comprises the amino acid sequence as shown in SEQ ID NO: 11.

In a further embodiment of the antibody-drug conjugate, the first antigen-binding domain and the second antigen-binding domain are Fabs, and the Fab of the second antigen-binding domain comprises domain swaps in a form of CrossMab.

In a further embodiment of the antibody-drug conjugate, the bispecific antibody comprises a polypeptide chain V-A, a polypeptide chain V-B, a polypeptide chain V-C and a polypeptide chain V-D; wherein the polypeptide chain V-A comprises the first VL and a light chain constant region; the polypeptide chain V-B comprises: the first VH, a heavy chain CH1 region and the first Fc domain monomer (or the second Fc domain monomer), and the polypeptide chain V-C comprises: the second VH, a light chain constant region and the second Fc domain monomer (or the first Fc domain monomer), the polypeptide chain V-D comprises: the second VL and a heavy chain CH1 region.

In a further embodiment of the antibody-drug conjugate, the polypeptide chain V-A comprises from N-terminus to C-terminus the first VL and the light chain constant region; the polypeptide chain V-B comprises from N-terminus to C-terminus the first VH, the heavy chain CH1 region and the first Fc domain monomer (or the second Fc domain monomer); the polypeptide chain V-C comprises from N-terminus to C-terminus the second VH, the light chain constant region and the second Fc domain monomer (or the first Fc domain monomer), and/or the polypeptide chain V-D comprises from N-terminus to C-terminus the second VL and the heavy chain CH1 region.

In a further embodiment of the antibody-drug conjugate, the adjacent domains of the polypeptide chain V-A are connected optionally with or without a linker, the adjacent domains of the polypeptide chain V-B are connected optionally with or without a linker, the adjacent domains of the polypeptide chain V-C are connected optionally with or without a linker, and/or the adjacent domains of the polypeptide chain V-D are connected optionally with or without a linker.

In a further embodiment of the antibody-drug conjugate, the linkers are each independently the same peptide linker or different peptide linkers (e.g., rigid peptide linkers or flexible peptide linkers); or the peptide linkers are each independently selected from peptide linkers comprising one or more glycines (G) and/or serines (S), for example, having the structure shown in (GGGGS)ₙ, wherein n is selected from 1, 2, 3, 4, 5, 6, 7, 8, 9 or 10 (SEQ ID NOs: 55-58 or 65-70); or the peptide linkers each independently comprise the amino acid sequence shown in SEQ ID NOs: 55-58, 61, or 65-70.

In a further embodiment of the antibody-drug conjugate, the light chain constant region comprises the amino acid sequence as shown in SEQ ID NO: 53 or SEQ ID NO: 63, and/or the heavy chain CH1 region comprises the amino acid sequence as shown in SEQ ID NO: 54

In a further embodiment of the antibody-drug conjugate, the polypeptide chain V-A comprises the amino acid sequence as shown in SEQ ID NO: 1, the polypeptide chain V-B comprises the amino acid sequence as shown in SEQ ID NO: 9; the polypeptide chain V-C comprises the amino acid sequence as shown in SEQ ID NO: 13, and/or the sequence of the polypeptide chain V-D is shown in SEQ ID NO: 12.

In a further embodiment of the antibody-drug conjugate, the first antigen-binding domain and the second antigen-binding domain are Fabs, and the Fab of the first antigen-binding domain comprises domain swaps in a form of CrossMab.

In a further embodiment of the antibody-drug conjugate, the bispecific antibody comprises a polypeptide chain VI-A, a polypeptide chain VI-B, a polypeptide chain VI-C and a polypeptide chain VI-D; wherein the polypeptide chain VI-A comprises the second VL and a light chain constant region; the polypeptide chain VI-B comprises: the second VH, a heavy chain CH1 region and the first Fc domain monomer (or the second Fc domain monomer); the polypeptide chain VI-C comprises: the first VH, a light chain constant region and the second Fc domain monomer (or the first Fc domain monomer); the polypeptide chain VI-D comprises: the first VL and a heavy chain CH1 region.

In a further embodiment of the antibody-drug conjugate, the polypeptide chain VI-A comprises from N-terminus to C-terminus the second VL and the light chain constant region, and the polypeptide chain VI-B comprises from N-terminus to C-terminus the second VH, the CH1 region of the heavy chain, and the first Fc domain monomer (or the second Fc domain monomer), the polypeptide chain VI-C comprises from N-terminus to C-terminus the first VH, the light chain constant region and the second Fc domain monomer (or the first Fc domain monomer), and/or the polypeptide chain VI-D from N-terminus to C-terminus comprises: the first VL and the heavy chain CH1 region.

In a further embodiment of the antibody-drug conjugate, the adjacent domains of the polypeptide chain VI-A are connected optionally with or without a linker; the adjacent domains of the polypeptide chain VI-B are connected optionally with or without a linker; the adjacent domains of the polypeptide chain VI-C are connected optionally with or without a linker; and/or the adjacent domains of the polypeptide chain VI-D are connected optionally with or without a linker.

In a further embodiment of the antibody-drug conjugate, the linkers are each independently the same peptide linker or different peptide linkers (e.g., rigid peptide linkers or flexible peptide linkers); or the peptide linkers are each independently selected from peptide linkers comprising one or more glycines (G) and/or serines (S), for example, having the structure shown in (GGGGS)ₙ, wherein n is selected from 1, 2, 3, 4, 5, 6, 7, 8, 9 or 10 (SEQ ID NOs: 55-58 or 65-70); or the peptide linkers each independently comprise the amino acid sequence shown in SEQ ID NOs: 55-58, 61, or 65-70.

In a further embodiment of the antibody-drug conjugate, the light chain constant region comprises the amino acid sequence as shown in SEQ ID NO: 53 or SEQ ID NO: 63, and/or the heavy chain CH1 region comprises the amino acid sequence as shown in SEQ ID NO: 54.

In a further embodiment of the antibody-drug conjugate, both the first antigen-binding domain and the second antigen-binding domain are scFvs.

In a further embodiment of the antibody-drug conjugate, the bispecific antibody comprises a polypeptide chain VII-A and polypeptide chain VII-B; wherein the polypeptide chain VII-A comprises: the first VL, the first VH and the first Fc domain monomer (or the second Fc domain monomer), the polypeptide chain VII-B comprises: the second VL, the second VH and the second Fc domain monomer (or the first Fc domain monomer).

In a further embodiment of the antibody-drug conjugate, the polypeptide chain VII-A comprises from N-terminus to C-terminus (i) the first VL, the first VH and the first Fc domain monomer (or the second Fc domain monomer); or (ii) the first VH, the first VL and the first Fc domain monomer (or the second Fc domain monomer); and/or the polypeptide chain VII-B comprises from N-terminus to C-terminus (i) the second VL, the second VH and the second Fc domain monomer (or the first Fc domain monomer), or (ii) the second VH, the second VL and the second Fc domain monomer (or the first Fc domain monomer).

In a further embodiment of the antibody-drug conjugate, the adjacent domains of the polypeptide chain VII-A are connected optionally with or without a linker, and/or the adjacent domains of the polypeptide chain VII-B are connected optionally with or without a linker.

In a further embodiment of the antibody-drug conjugate, the linkers are each independently the same peptide linker or different peptide linkers (e.g., rigid peptide linkers or flexible peptide linkers); or the peptide linkers are each independently selected from peptide linkers comprising one or more glycines (G) and/or serines (S), for example, having the structure shown in (GGGGS)ₙ, wherein n is selected from 1, 2, 3, 4, 5, 6, 7, 8, 9 or 10 (SEQ ID NOs: 55-58 or 65-70).

In a further embodiment of the antibody-drug conjugate, the peptide linkers each independently comprise the amino acid sequence shown in SEQ ID NOs: 55-58, 61, or 65-70.

In a further embodiment of the antibody-drug conjugate, the polypeptide chain VII-A comprises the amino acid sequence as shown in SEQ ID NO: 14, and/or the polypeptide chain VII-B comprises the amino acid sequence as shown in SEQ ID NO: 8.

In a further embodiment of the antibody-drug conjugate, the bispecific antibody or antigen-binding fragment thereof comprises:
(1) polypeptide chain I-A comprising the amino acid sequence shown in SEQ ID NO: 1, polypeptide chain I-B comprising the amino acid sequence shown in SEQ ID NO: 2, and polypeptide chain I-C comprising the amino acid sequence shown in SEQ ID NO: 3;
(2) polypeptide chain I-A comprising the amino acid sequence shown in SEQ ID NO: 1, polypeptide chain I-B comprising the amino acid sequence shown in SEQ ID NO: 9, and polypeptide chain I-C comprising the amino acid sequence shown in SEQ ID NO: 10;
(3) polypeptide chain II-A comprising the amino acid sequence shown in SEQ ID NO: 4, polypeptide chain II-B comprising the amino acid sequence shown in SEQ ID NO: 5, and polypeptide chain II-C comprising the amino acid sequence shown in SEQ ID NO: 6;
(4) polypeptide chain II-A comprising the amino acid sequence shown in SEQ ID NO: 4, polypeptide chain II-B comprising the amino acid sequence shown in SEQ ID NO: 7, and polypeptide chain II-C comprising the amino acid sequence shown in SEQ ID NO: 8;
(5) polypeptide chain IV-A comprising the amino acid sequence shown in SEQ ID NO: 4, polypeptide chain IV-B comprising the amino acid sequence shown in SEQ ID NO: 7, and polypeptide chain IV-C comprising the amino acid sequence shown in SEQ ID NO: 11;
(6) polypeptide chain V-A comprising the amino acid sequence shown in SEQ ID NO: 1, polypeptide chain V-B comprising the amino acid sequence shown in SEQ ID NO: 9, polypeptide chain V-C comprising the amino acid sequence shown in SEQ ID NO: 13, and polypeptide chain V-D comprising the amino acid sequence as shown in SEQ ID NO: 12; or
(7) polypeptide chain VII-A comprising the amino acid sequence shown in SEQ ID NO: 14, and polypeptide chain VII-B comprising the amino acid sequence shown in SEQ ID NO: 8.

In a further embodiment of the antibody-drug conjugate, the antibody-drug conjugate has an enhanced tumor suppressive effect relative to a monospecific anti-c-MET antibody and/or a monospecific anti-EGFR antibody; wherein the amino acid sequences of the CDRs of the monospecific anti-c-MET antibody are identical to the amino acid sequences of the CDRs of the first antigen-binding domain, and the amino acid sequences of the CDRs of the monospecific anti-EGFR antibody are identical to the amino acid sequences of the CDRs of the second antigen-binding domain.

In a further embodiment of the antibody-drug conjugate, the tumor suppressive effect comprises: inhibition of EGFR and c-MET signaling pathways, antibody-dependent cell-mediated cytotoxicity (ADCC) activity and/or complement-dependent cytotoxicity (CDC) activity.

In a further embodiment of the antibody-drug conjugate, M comprises

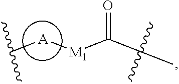

and wherein ring A is a 5-6 membered aliphatic heterocyclic ring or a 5-20 membered aromatic ring system, and the aliphatic heterocyclic ring and aromatic ring system are optionally replaced by one or more groups independently selected from the group consisting of oxo (═O), halogen, cyano, amino, carboxyl, thiol, and C₁₋₆ alkyl; and M₁ is selected from single bond and C₁₋₂₀ alkylene, C₂₋₂₀ alkenylene, C₂₋₂₀ alkynylene, or amine group.

In a further embodiment of the antibody-drug conjugate, M comprises

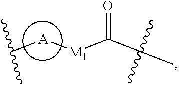

wherein ring A is a 5-membered aliphatic heterocyclic ring, a 6-membered heteroaromatic ring, or a polycyclic ring formed by connecting more than one 6-membered aromatic heterocyclic ring and a benzene ring through a single bond or by connecting more than one 6-membered heteroaromatic rings through single bonds, and wherein the aliphatic heterocyclic ring is optionally substituted by one or more groups selected from oxo (═O), halogen, and C₁₋₄ Alkyl group substitution; and M₁ is selected from single bond, C₁₋₂₀ Alkylene, C₂₋₂₀ alkenylene, C₂₋₂₀ alkynylene or amine group.

In a further embodiment of the antibody-drug conjugate, M is

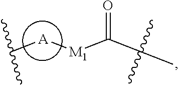

wherein ring A is selected from

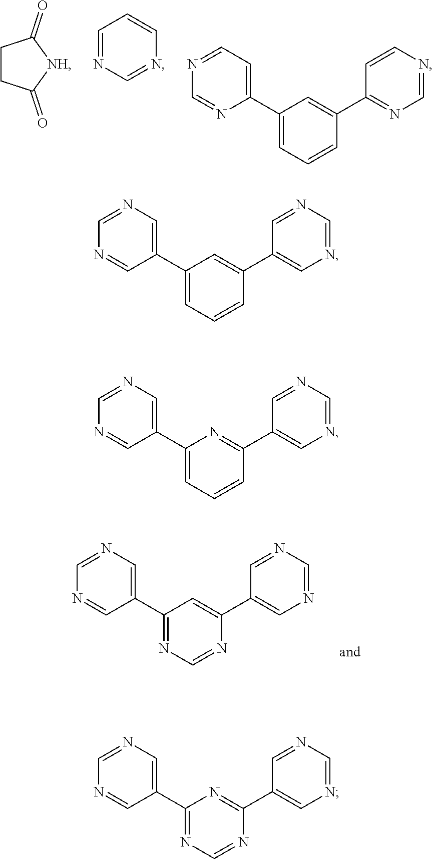

and M₁ is selected from a single bond, C₁₋₆ alkylene, C₂₋₆ alkenylene, C₂₋₆ alkynylene, and amine group.

In a further embodiment of the antibody-drug conjugate, M is selected from

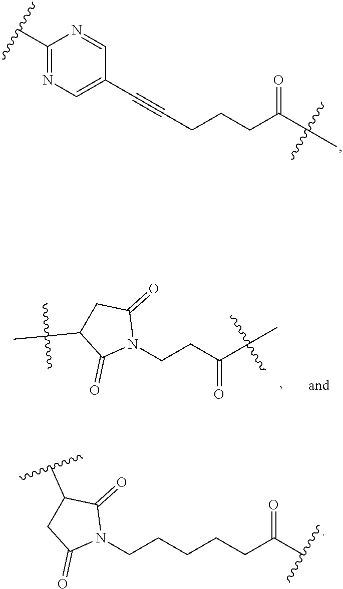 [SMILES: *C(=O)CCCC#Cc1cnc(*)nc1 ; *C(=O)CCN1C(=O)CC(*)C1=O ; *C(=O)CCCCCN1C(=O)CC(*)C1=O]

In a further embodiment of the antibody-drug conjugate, M is

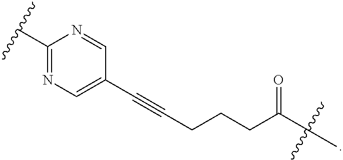 [SMILES: *C(=O)CCCC#Cc1cnc(*)nc1]

In a further embodiment of the antibody-drug conjugate, M is selected from

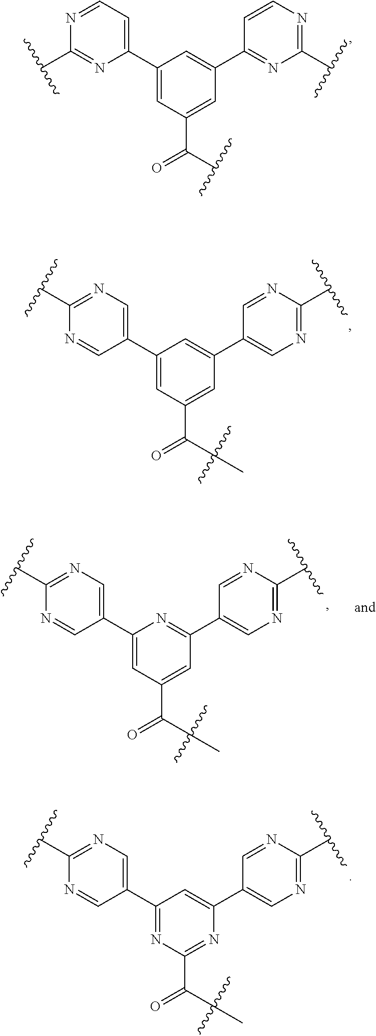 [SMILES: *C(=O)c1cc(-c2ccnc(*)n2)cc(-c2ccnc(*)n2)c1 ; *C(=O)c1cc(-c2cnc(*)nc2)cc(-c2cnc(*)nc2)c1 ; *C(=O)c1cc(-c2cnc(*)nc2)nc(-c2cnc(*)nc2)c1 ; *C(=O)c1nc(-c2cnc(*)nc2)cc(-c2cnc(*)nc2)n1]

In a further embodiment of the antibody-drug conjugate, M is selected from

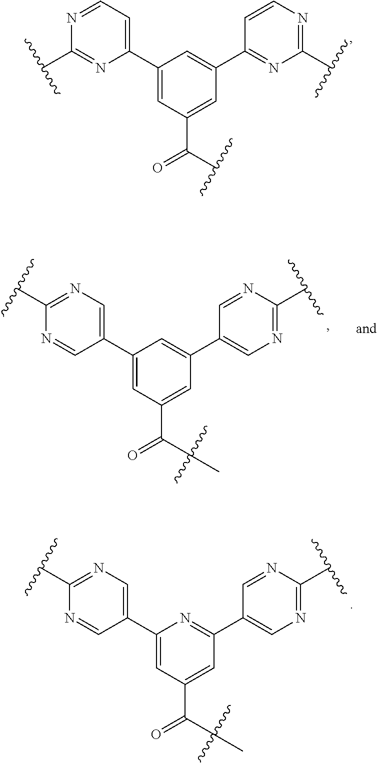 [SMILES: *C(=O)c1cc(-c2ccnc(*)n2)cc(-c2ccnc(*)n2)c1 ; *C(=O)c1cc(-c2cnc(*)nc2)cc(-c2cnc(*)nc2)c1 ; *C(=O)c1cc(-c2cnc(*)nc2)nc(-c2cnc(*)nc2)c1]

In a further embodiment of the antibody-drug conjugate, L is selected from a structure comprising one or more of the following: C₁₋₆ Alkylene, —N(R′)—, carbonyl, —O—, natural amino acids or unnatural amino acids and their analogs selected from Ala, Arg, Asn, Asp, Cit, Cys, Gln, Glu, Gly, His, Ile, Leu, Lys, Met, Phe, Pro, Ser, Thr, Trp, Tyr, Val, Lys, (COCH₂CH₂(OCH₂CH₂)ₛOCH₃)), and peptides comprising 1, 2, 3, or 4 amino acids,

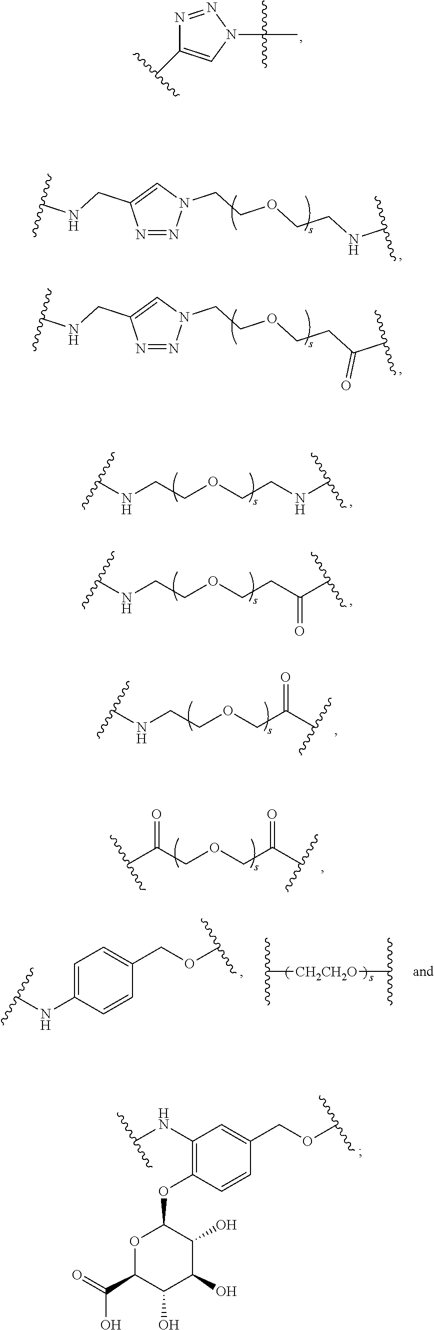

wherein R′ represents hydrogen, C₁₋₆ alkyl, or a polyethylene glycol fragment containing 1-10 ethylene oxide units; and s is selected from an integer of 1-20.

In a further embodiment of the antibody-drug conjugate, the peptide is selected from Ala-Ala, Ala-Lys, Ala-Lys(Ac), Ala-Pro, Gly-Glu, Gly-Gly, Phe-Lys, Phe-Lys(Ac), Val-Ala, Val-Lys, Val-Lys(Ac), Val-Cit, Ala-Ala-Ala, Ala-Ala-Asn, Leu-Ala-Glu, Gly-Gly-Arg, Gly-Glu-Gly, Gly-Gly-Gly, Gly-Ser-Lys, Glu-Val-Ala, Glu-Val-Cit, Ser-Ala-Pro, Val-Leu-Lys, Val-Lys-Ala, Val-Lys-Gly, Gly-Gly-Phe-Gly (GGFG (SEQ ID NO: 71)), Gly-Gly-Val-Ala (GGVA (SEQ ID NO: 72), Gly-Phe-Leu-Gly (GFLG (SEQ ID NO: 73)), Glu-Ala-Ala-Ala (EAAA (SEQ ID NO: 74)), and Gly-Gly-Gly-Gly-Gly (GGGGG (SEQ ID NO: 75)).

In a further embodiment of the antibody-drug conjugate, L is selected from a structure comprising one or more of the following: C₁₋₆ Alkylene, carbonyl, —NH—, Ala-Ala, Ala-Lys, Ala-Pro, Gly-Glu, Gly-Gly, Phe-Lys, Val-Ala, Val-Lys, Val-Cit, Ala-Ala-Ala, Ala-Ala-Asn, Leu-Ala-Glu, Gly-Gly-Arg, Gly-Glu-Gly, Gly-Gly-Gly, Gly-Ser-Lys, Glu-Val-Ala, Glu-Val-Cit, Ser-Ala-Pro, Val-Leu-Lys, Val-Lys-Ala, Val-Lys-Gly, Gly-Gly-Phe-Gly (GGFG (SEQ ID NO: 71)), Gly-Gly-Val-Ala (GGVA (SEQ ID NO: 72), Gly-Phe-Leu-Gly (GFLG (SEQ ID NO: 73)), Glu-Ala-Ala-Ala (EAAA (SEQ ID NO: 74)), and Gly-Gly-Gly-Gly-Gly (GGGGG (SEQ ID NO: 75)),

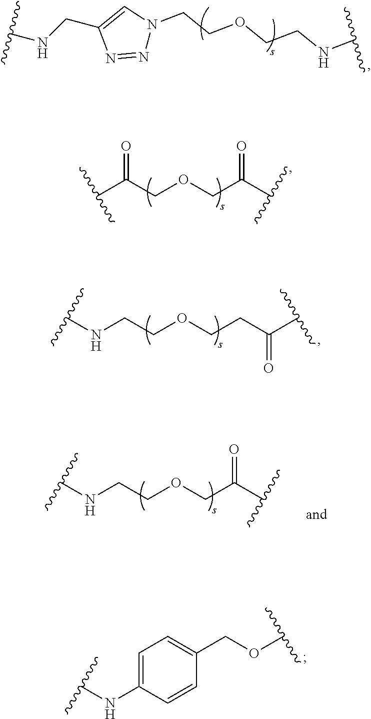

wherein s is selected from an integer of 1-20.

In a further embodiment of the antibody-drug conjugate, L is selected from a structure consisting of one or more of the following:

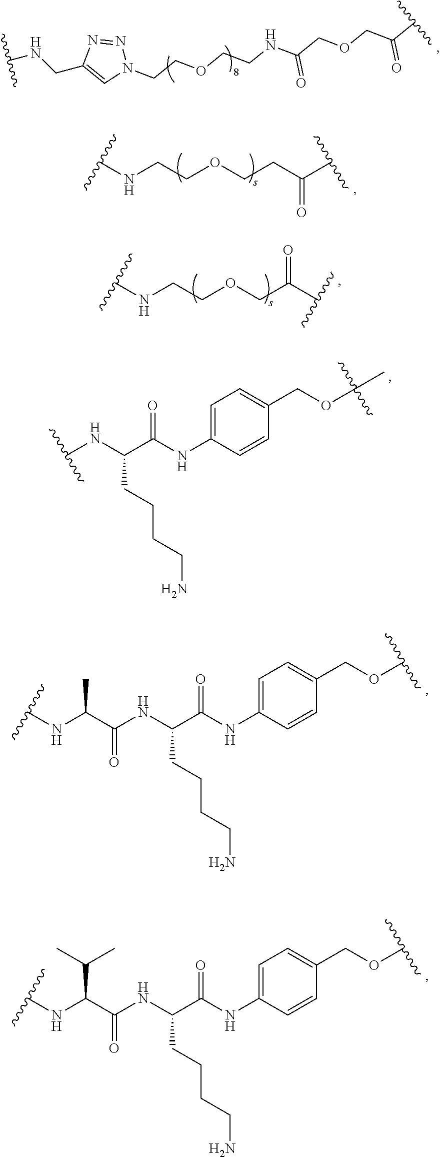

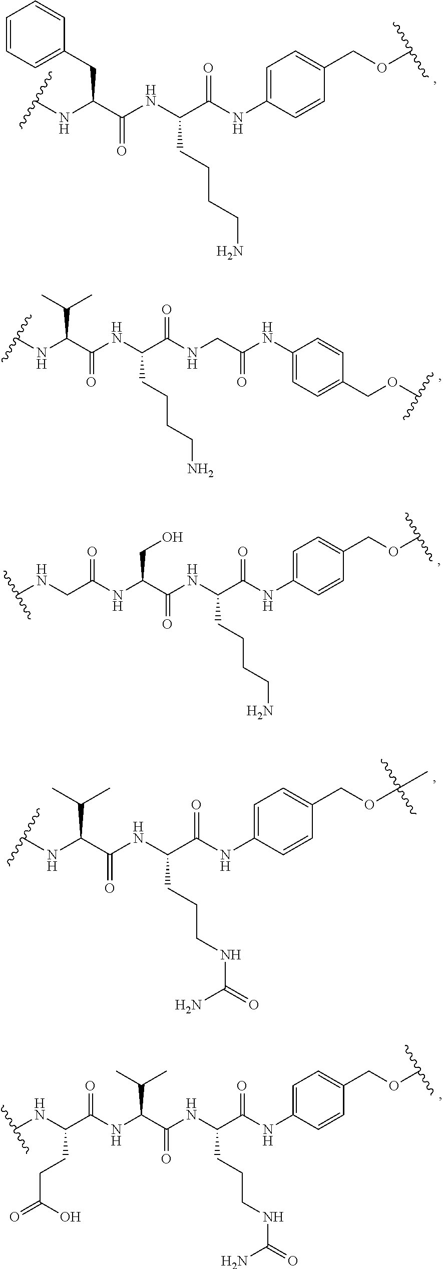 [SMILES: *N[C@@H](Cc1ccccc1)C(=O)N[C@@H](CCCCN)C(=O)Nc1ccc(CO*)cc1 ; *N[C@H](C(=O)N[C@@H](CCCCN)C(=O)NCC(=O)Nc1ccc(CO*)cc1)C(C)C ; *NCC(=O)N[C@@H](CO)C(=O)N[C@@H](CCCCN)C(=O)Nc1ccc(CO*)cc1 ; *N[C@H](C(=O)N[C@@H](CCCNC(N)=O)C(=O)Nc1ccc(CO*)cc1)C(C)C ; *N[C@@H](CCC(=O)O)C(=O)N[C@H](C(=O)N[C@@H](CCCNC(N)=O)C(=O)Nc1ccc(CO*)cc1)C(C)C]

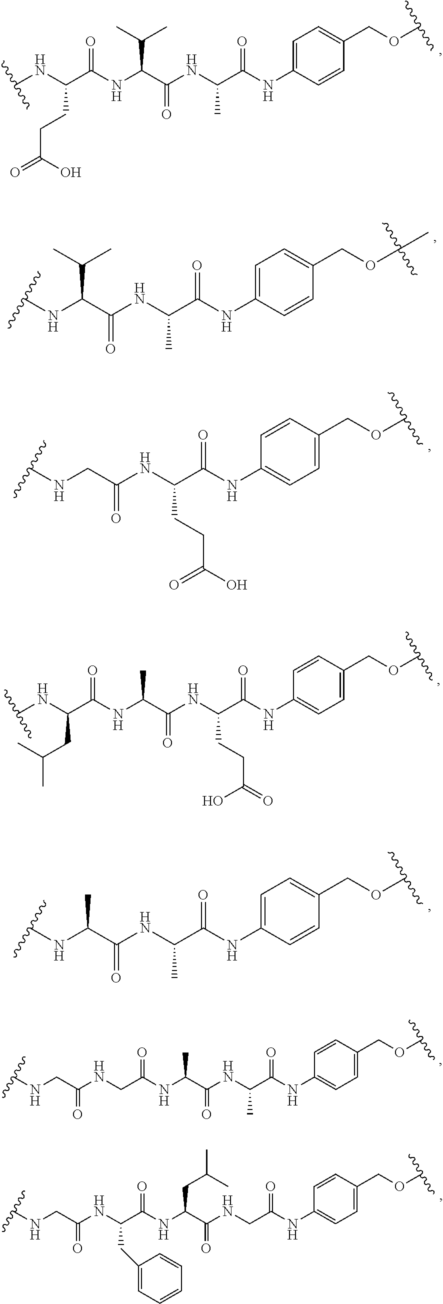

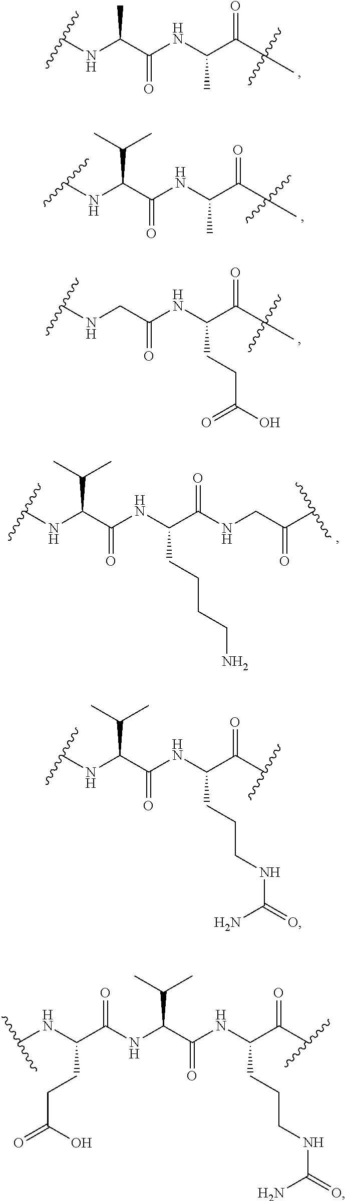

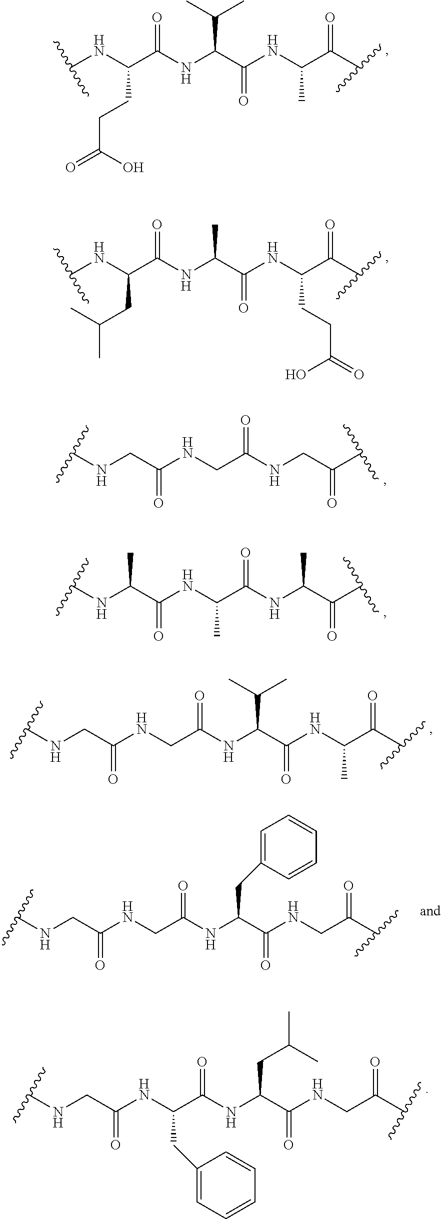

In a further embodiment of the antibody-drug conjugate, L is selected from the following structures:

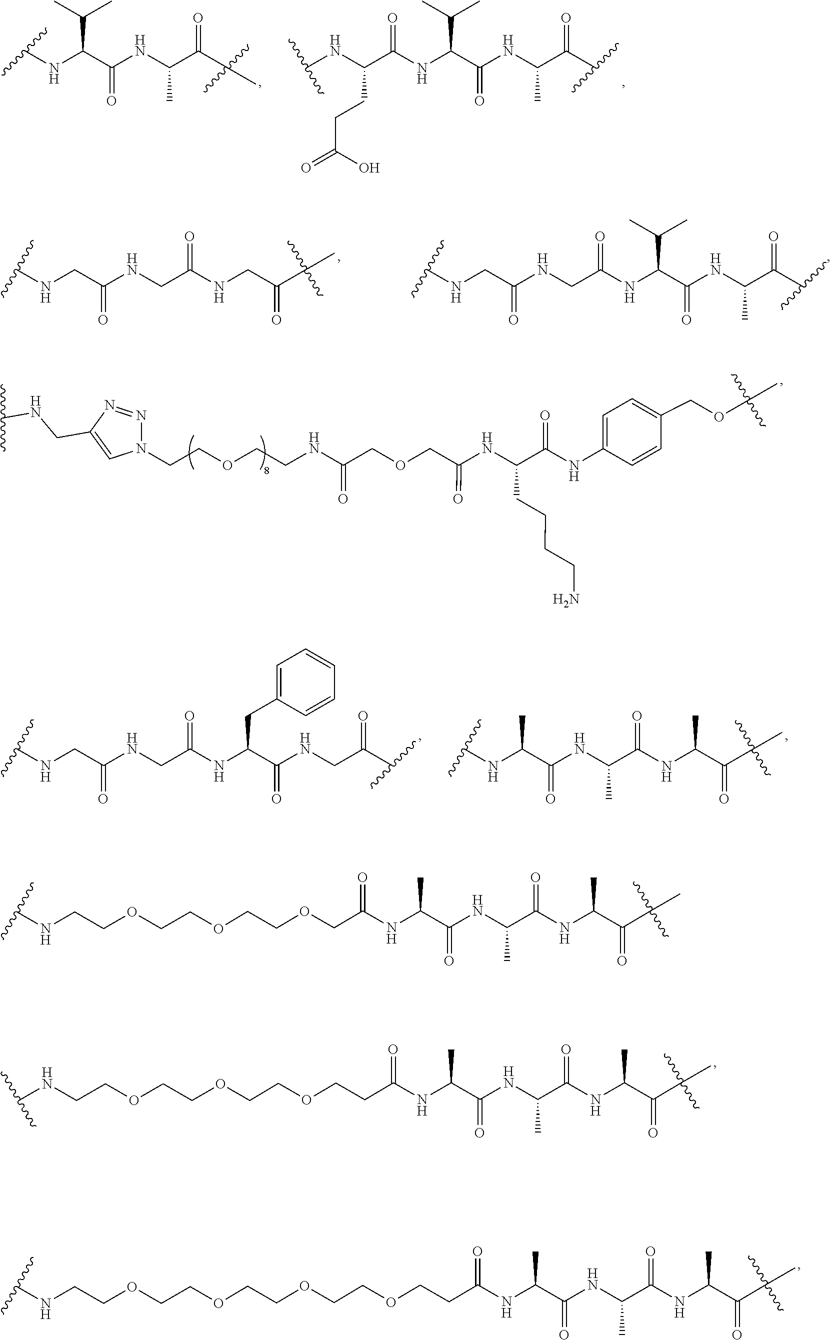

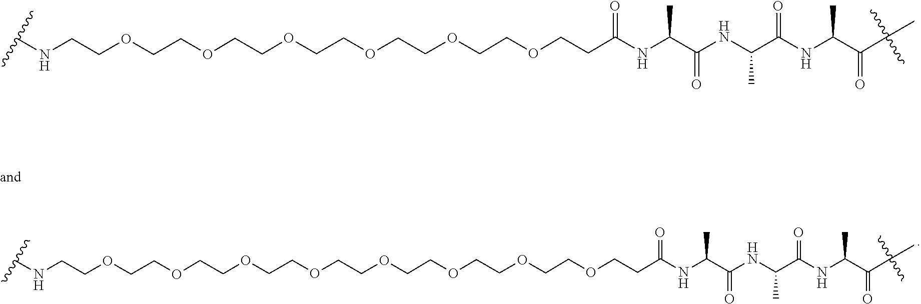 [SMILES: *NCCOCCOCCOCCOCCOCCOCCC(=O)N[C@@H](C)C(=O)N[C@@H](C)C(=O)N[C@@H](C)C(*)=O ; *NCCOCCOCCOCCOCCOCCOCCOCCOCCC(=O)N[C@@H](C)C(=O)N[C@@H](C)C(=O)N[C@@H](C)C(*)=O]

In a further embodiment of the antibody-drug conjugate, L is selected from the following structures:

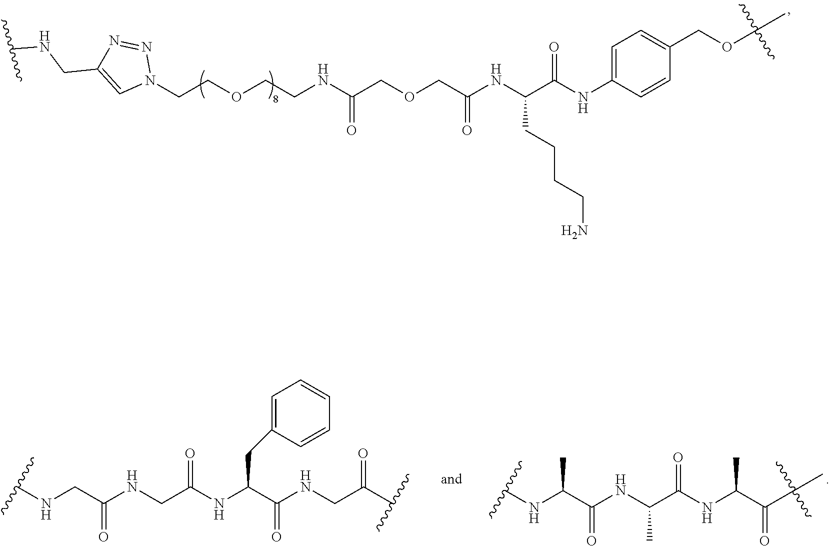

In a further embodiment of the antibody-drug conjugate, L is selected from the following structures:

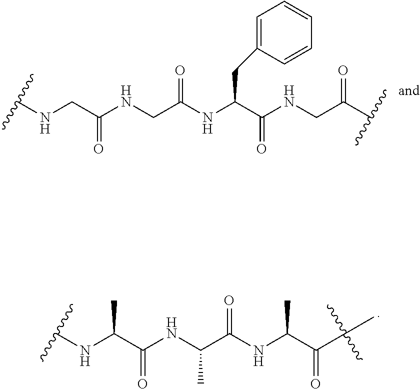 [SMILES: *NCC(=O)NCC(=O)N[C@@H](Cc1ccccc1)C(=O)NCC(*)=O ; "and" ; *N[C@@H](C)C(=O)N[C@@H](C)C(=O)N[C@@H](C)C(*)=O]

In a further embodiment of the antibody-drug conjugate, L is selected from the following structures:

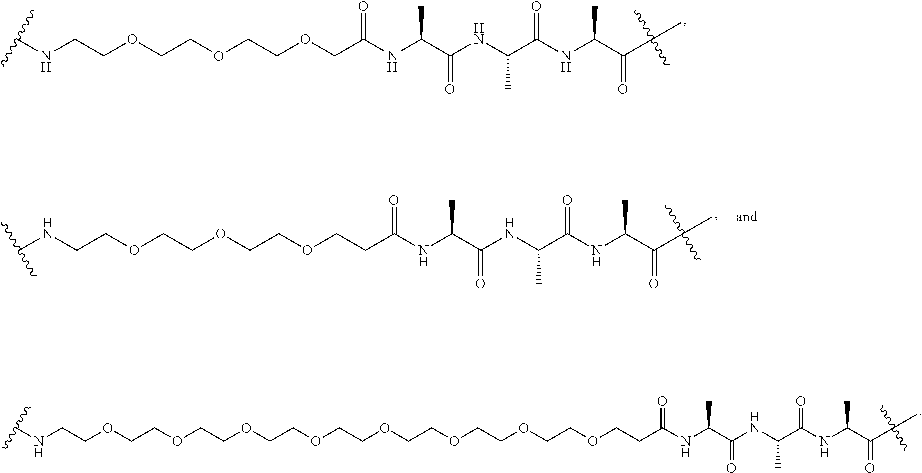 [SMILES: *NCCOCCOCCOCC(=O)N[C@@H](C)C(=O)N[C@@H](C)C(=O)N[C@@H](C)C(*)=O ; *NCCOCCOCCOCCC(=O)N[C@@H](C)C(=O)N[C@@H](C)C(=O)N[C@@H](C)C(*)=O ; *NCCOCCOCCOCCOCCOCCOCCOCCOCCC(=O)N[C@@H](C)C(=O)N[C@@H](C)C(=O)N[C@@H](C)C(*)=O]

In a further embodiment of the antibody-drug conjugate, E is a single bond or selected from the following structures: —NCH₂—,

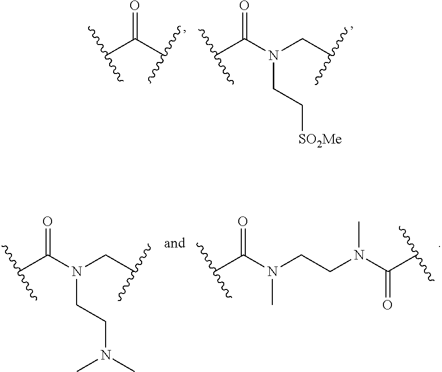

In a further embodiment of the antibody-drug conjugate, E is a single bond, —NHCH₂—,

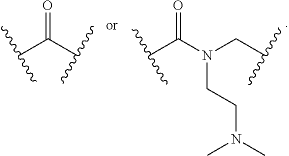

In a further embodiment of the antibody-drug conjugate, E is —NHCH₂— or

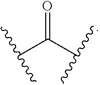 [SMILES: *C(*)=O]

In a further embodiment of the antibody-drug conjugate, E is —NHCH₂— or a single bond.

In a further embodiment of the antibody-drug conjugate, E is

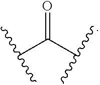 [SMILES: *C(*)=O]

In a further embodiment of the antibody-drug conjugate,

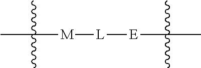

is selected from the following structures:

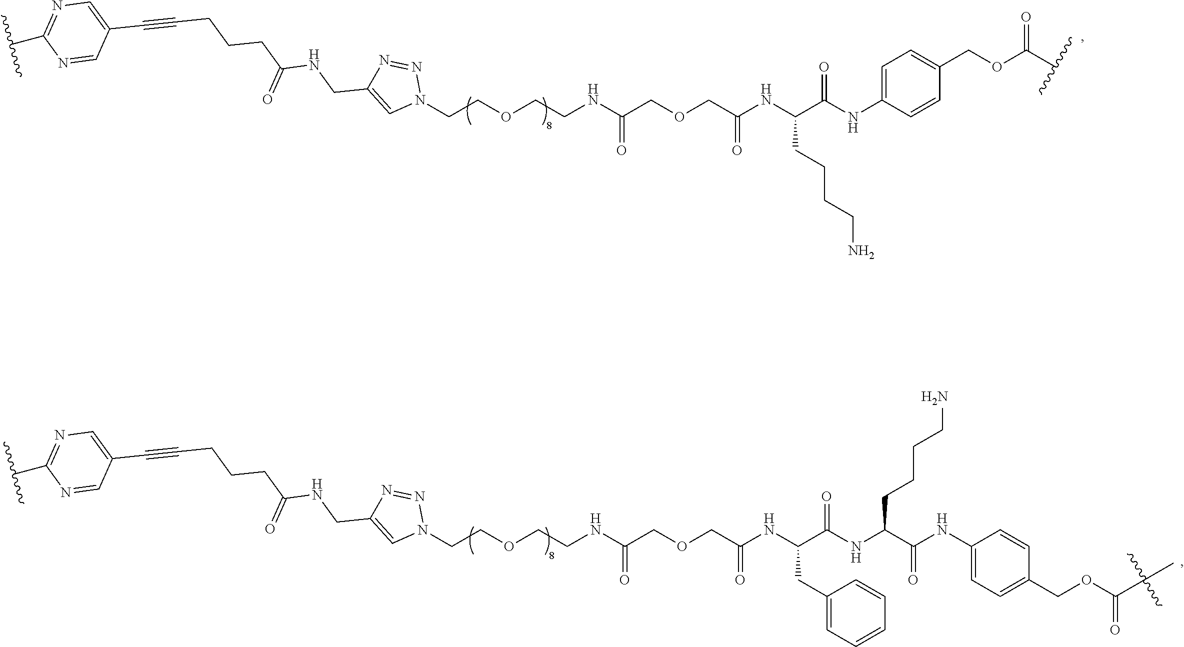

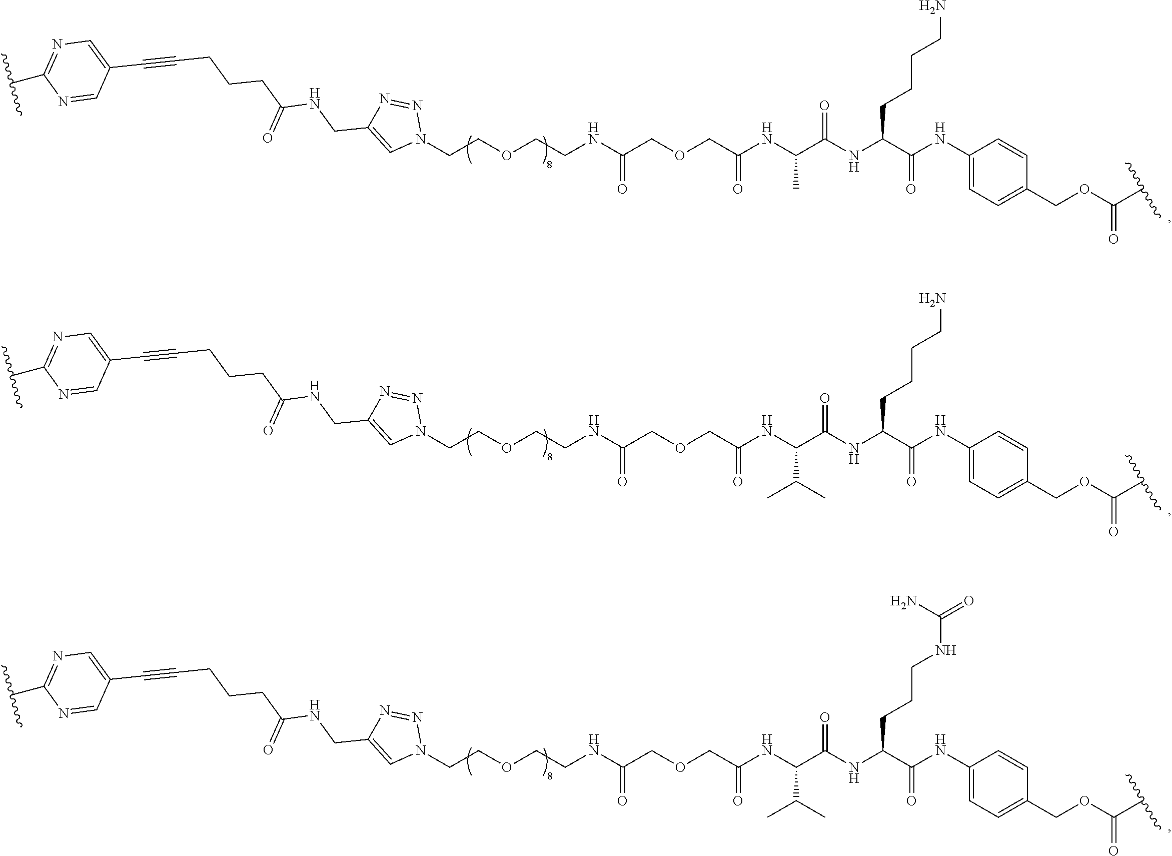

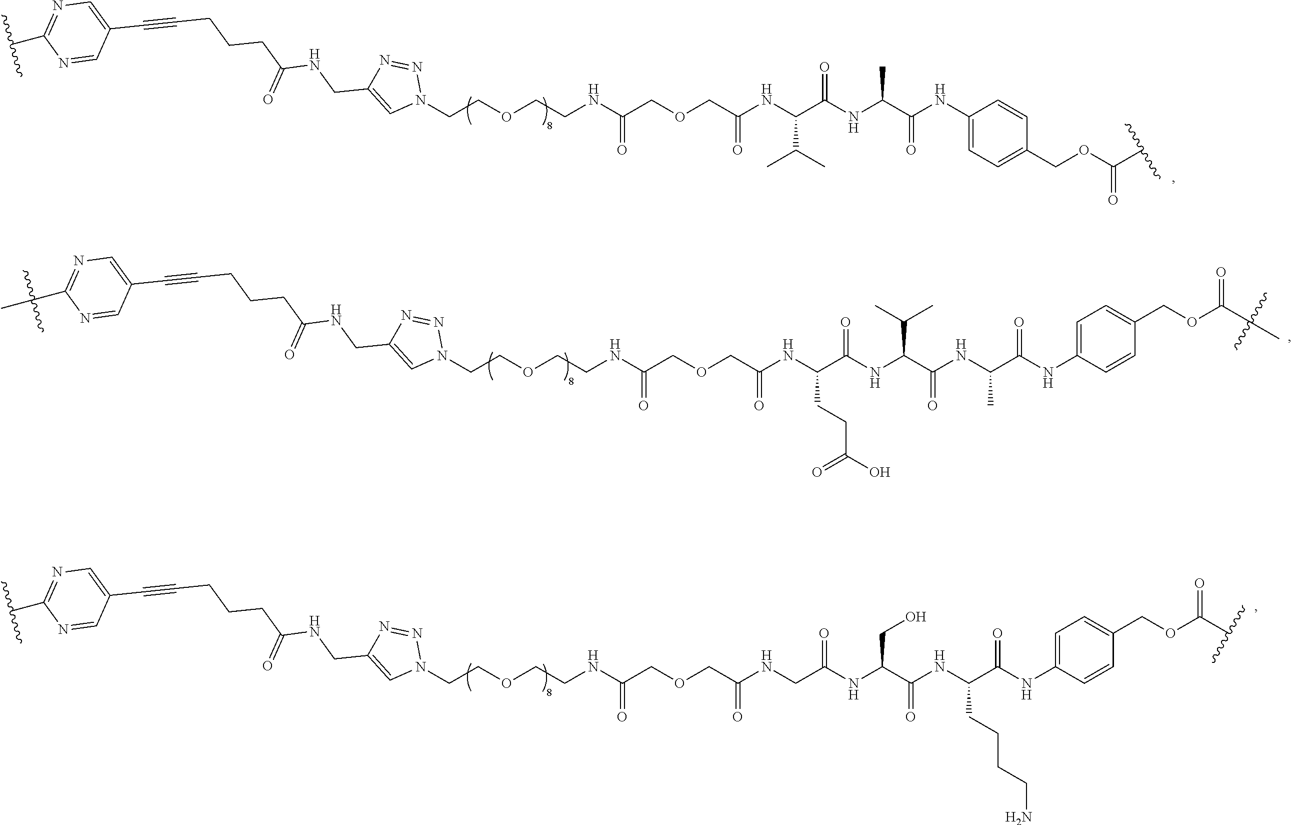

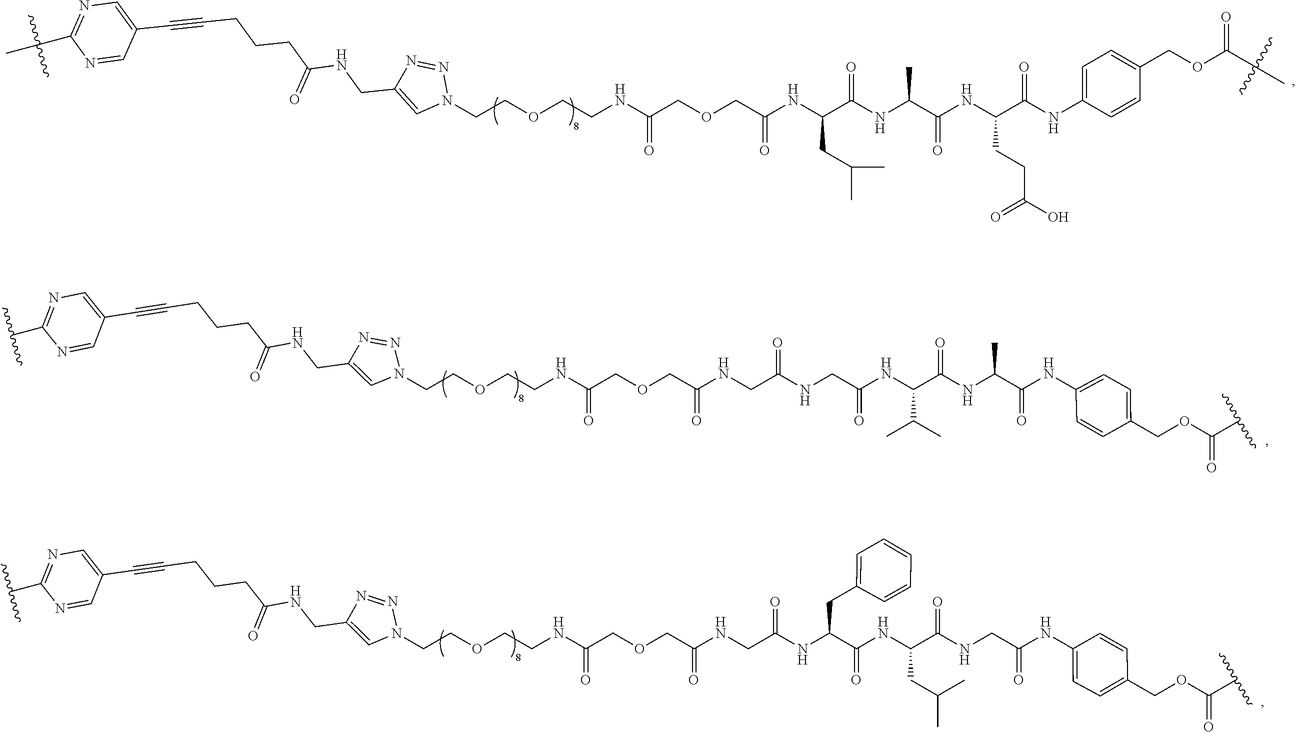

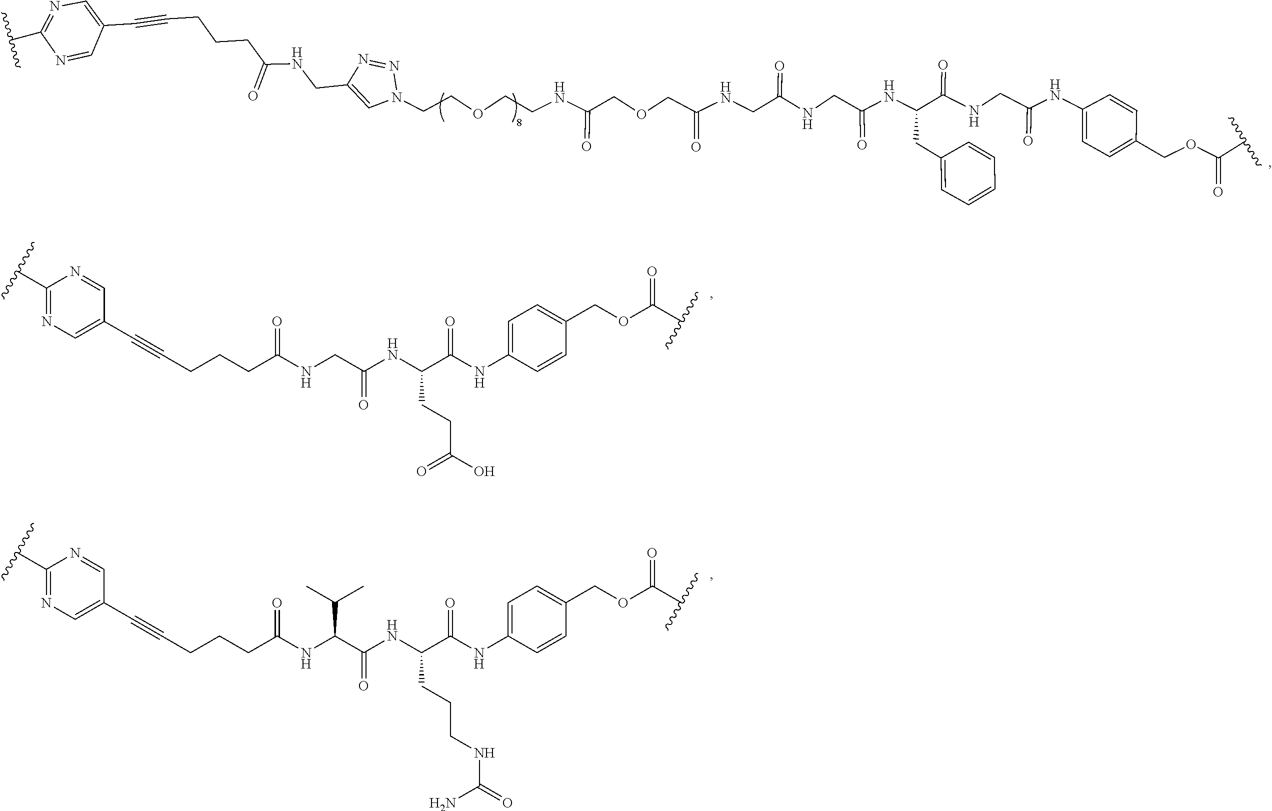

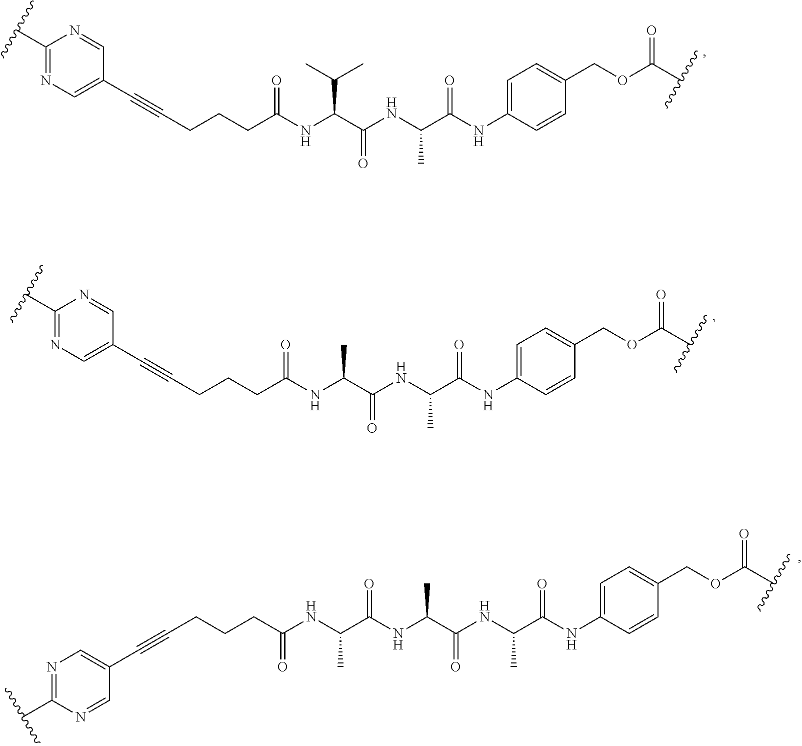 [SMILES: *C(=O)OCc1ccc(NC(=O)[C@H](C)NC(=O)[C@@H](NC(=O)CCCC#Cc2cnc(*)nc2)C(C)C)cc1 ; *C(=O)OCc1ccc(NC(=O)[C@H](C)NC(=O)[C@H](C)NC(=O)CCCC#Cc2cnc(*)nc2)cc1 ; *C(=O)OCc1ccc(NC(=O)[C@H](C)NC(=O)[C@H](C)NC(=O)[C@H](C)NC(=O)CCCC#Cc2cnc(*)nc2)cc1]

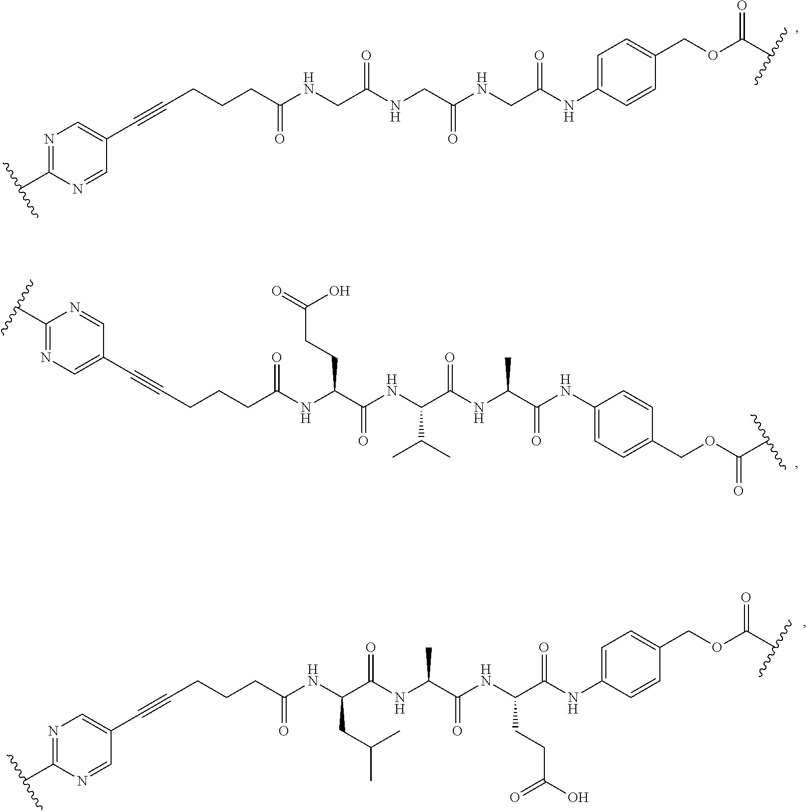 [SMILES: *C(=O)OCc1ccc(NC(=O)CNC(=O)CNC(=O)CNC(=O)CCCC#Cc2cnc(*)nc2)cc1 ; *C(=O)OCc1ccc(NC(=O)[C@H](C)NC(=O)[C@@H](NC(=O)[C@H](CCC(=O)O)NC(=O)CCCC#Cc2cnc(*)nc2)C(C)C)cc1 ; *C(=O)OCc1ccc(NC(=O)[C@H](CCC(=O)O)NC(=O)[C@H](C)NC(=O)[C@@H](CC(C)C)NC(=O)CCCC#Cc2cnc(*)nc2)cc1]

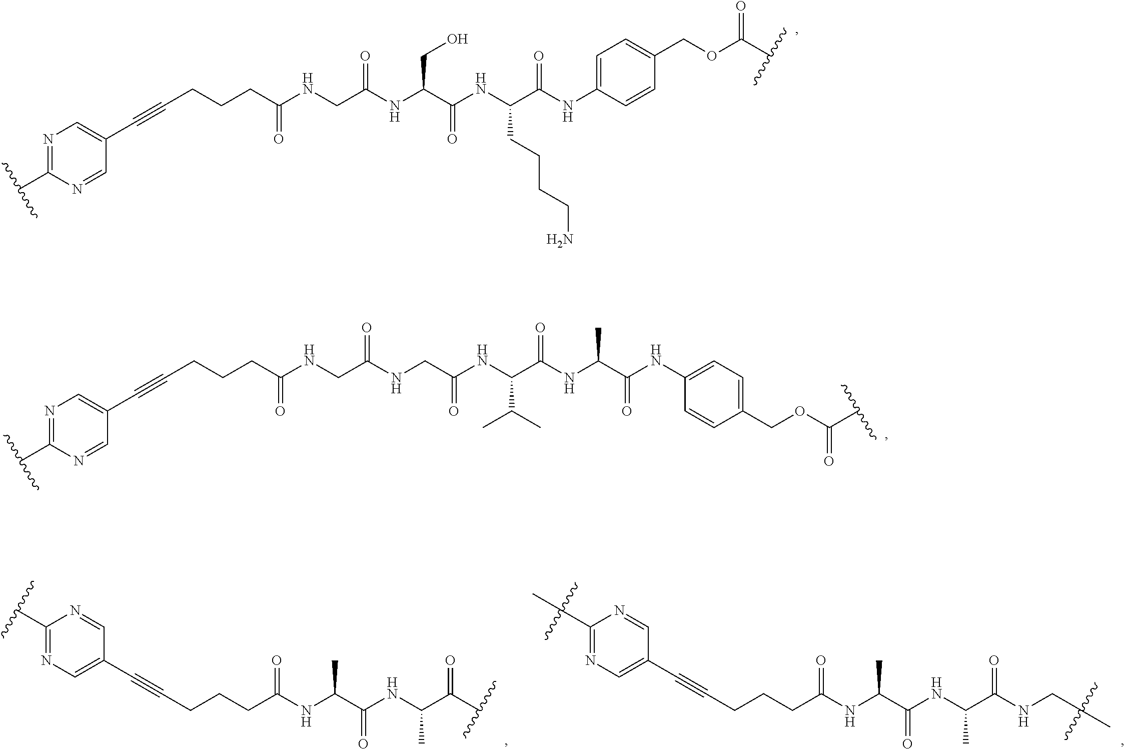 [SMILES: *C(=O)OCc1ccc(NC(=O)[C@H](CCCCN)NC(=O)[C@H](CO)NC(=O)CNC(=O)CCCC#Cc2cnc(*)nc2)cc1 ; *C(=O)OCc1ccc(NC(=O)[C@H](C)NC(=O)[C@@H](NC(=O)CNC(=O)CNC(=O)CCCC#Cc2cnc(*)nc2)C(C)C)cc1 ; *C(=O)[C@H](C)NC(=O)[C@H](C)NC(=O)CCCC#Cc1cnc(*)nc1 ; *CNC(=O)[C@H](C)NC(=O)[C@H](C)NC(=O)CCCC#Cc1cnc(*)nc1]

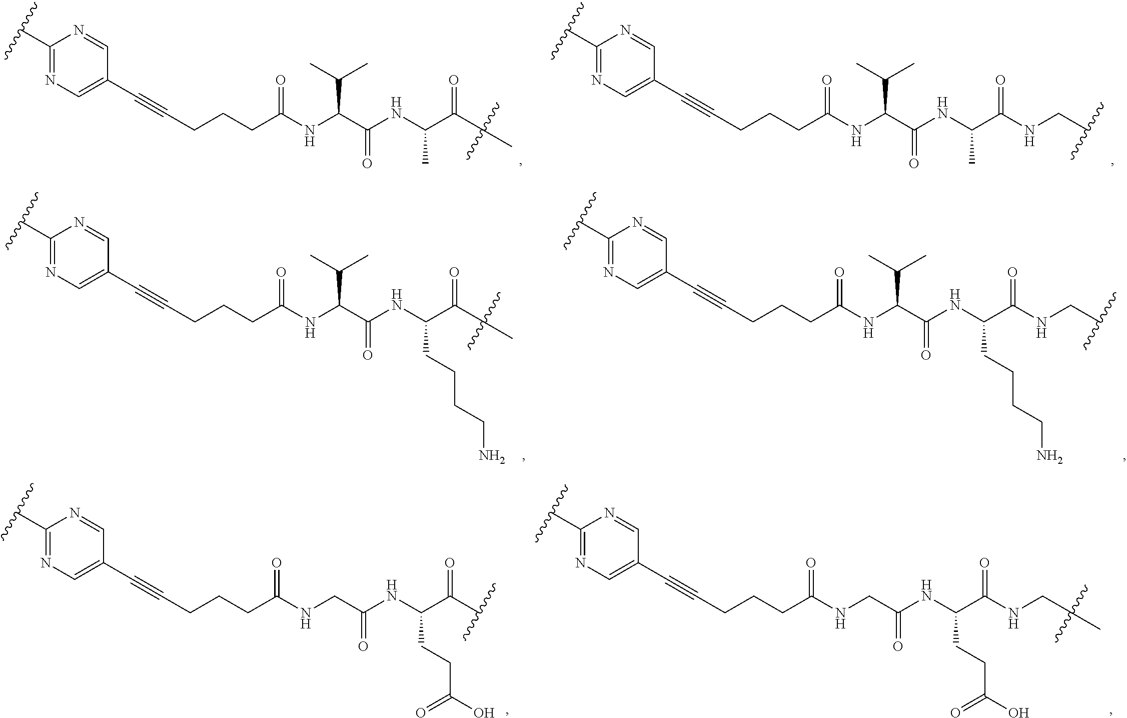 [SMILES: *C(=O)[C@H](C)NC(=O)[C@@H](NC(=O)CCCC#Cc1cnc(*)nc1)C(C)C ; *CNC(=O)[C@H](C)NC(=O)[C@@H](NC(=O)CCCC#Cc1cnc(*)nc1)C(C)C ; *C(=O)[C@H](CCCCN)NC(=O)[C@@H](NC(=O)CCCC#Cc1cnc(*)nc1)C(C)C ; *CNC(=O)[C@H](CCCCN)NC(=O)[C@@H](NC(=O)CCCC#Cc1cnc(*)nc1)C(C)C ; *C(=O)[C@H](CCC(=O)O)NC(=O)CNC(=O)CCCC#Cc1cnc(*)nc1 ; *CNC(=O)[C@H](CCC(=O)O)NC(=O)CNC(=O)CCCC#Cc1cnc(*)nc1]

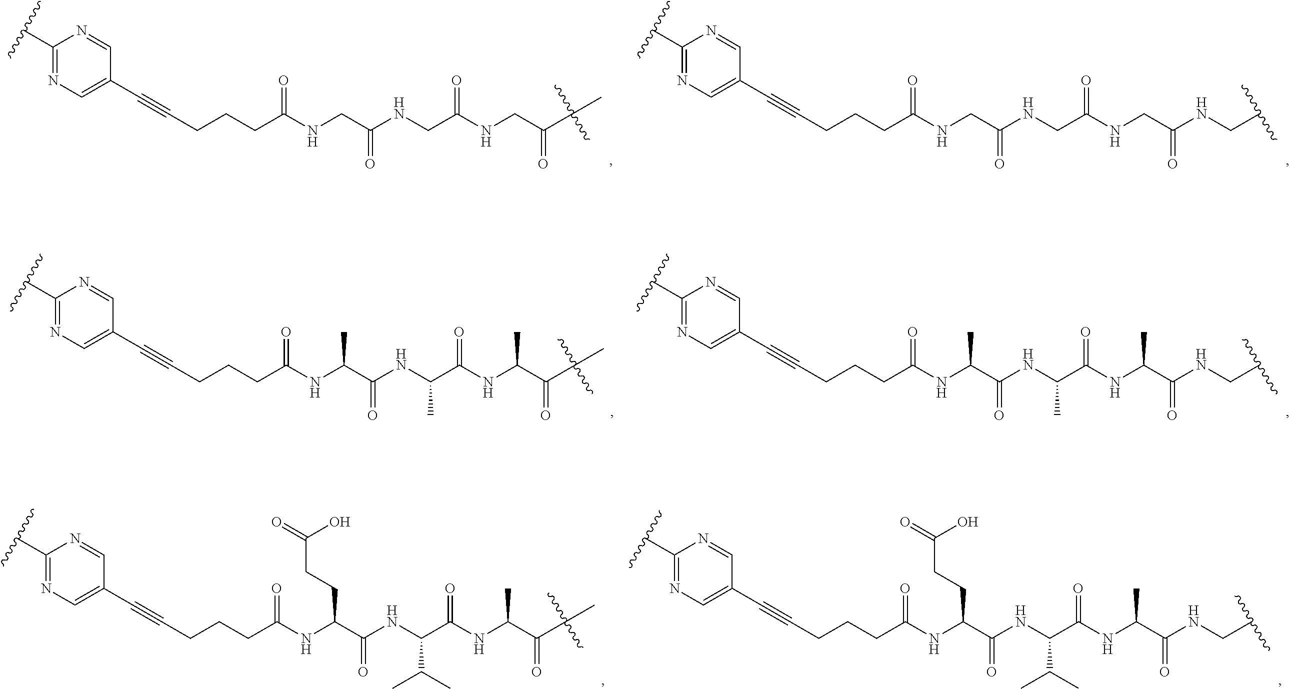 [SMILES: *C(=O)CNC(=O)CNC(=O)CNC(=O)CCCC#Cc1cnc(*)nc1 ; *CNC(=O)CNC(=O)CNC(=O)CNC(=O)CCCC#Cc1cnc(*)nc1 ; *C(=O)[C@H](C)NC(=O)[C@H](C)NC(=O)[C@H](C)NC(=O)CCCC#Cc1cnc(*)nc1 ; *CNC(=O)[C@H](C)NC(=O)[C@H](C)NC(=O)[C@H](C)NC(=O)CCCC#Cc1cnc(*)nc1 ; *C(=O)[C@H](C)NC(=O)[C@@H](NC(=O)[C@H](CCC(=O)O)NC(=O)CCCC#Cc1cnc(*)nc1)C(C)C ; *CNC(=O)[C@H](C)NC(=O)[C@@H](NC(=O)[C@H](CCC(=O)O)NC(=O)CCCC#Cc1cnc(*)nc1)C(C)C]

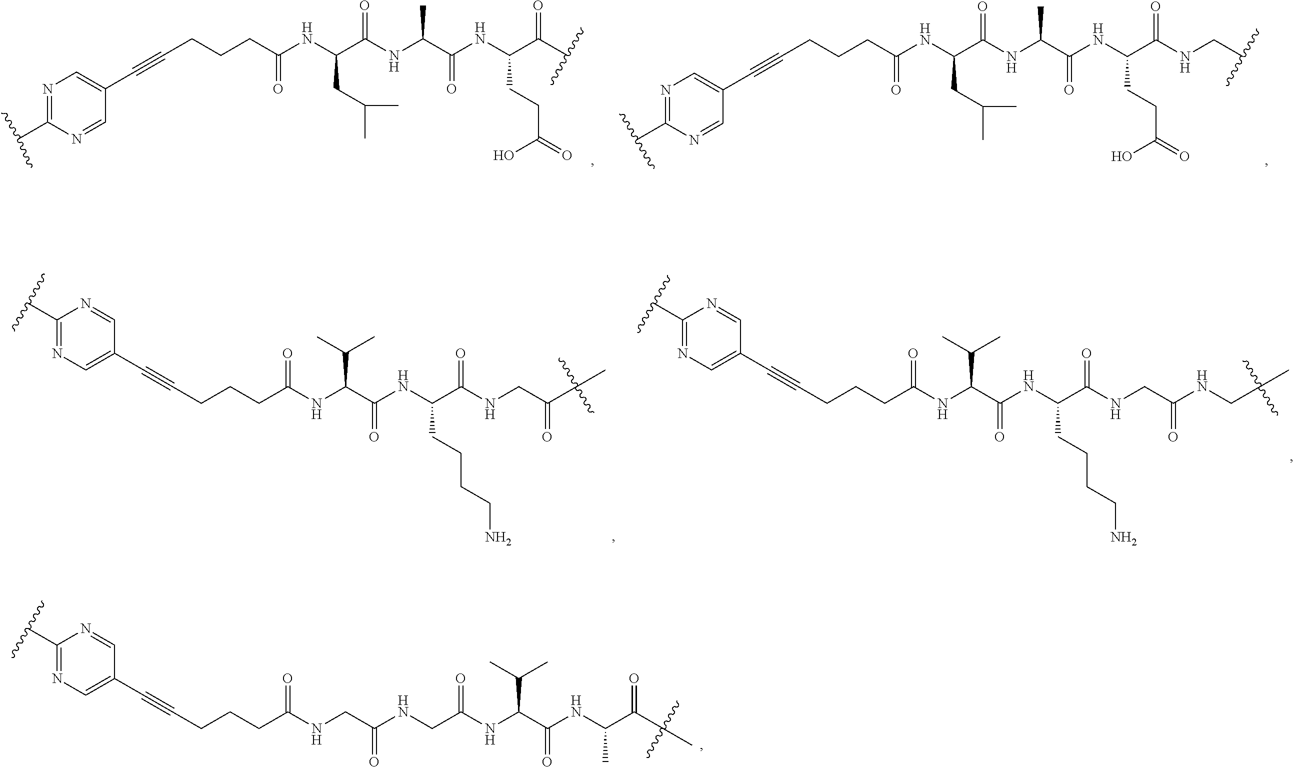 [SMILES: *C(=O)[C@H](CCC(=O)O)NC(=O)[C@H](C)NC(=O)[C@@H](CC(C)C)NC(=O)CCCC#Cc1cnc(*)nc1 ; *CNC(=O)[C@H](CCC(=O)O)NC(=O)[C@H](C)NC(=O)[C@@H](CC(C)C)NC(=O)CCCC#Cc1cnc(*)nc1 ; *C(=O)CNC(=O)[C@H](CCCCN)NC(=O)[C@@H](NC(=O)CCCC#Cc1cnc(*)nc1)C(C)C ; *CNC(=O)CNC(=O)[C@H](CCCCN)NC(=O)[C@@H](NC(=O)CCCC#Cc1cnc(*)nc1)C(C)C ; *C(=O)[C@H](C)NC(=O)[C@@H](NC(=O)CNC(=O)CNC(=O)CCCC#Cc1cnc(*)nc1)C(C)C]

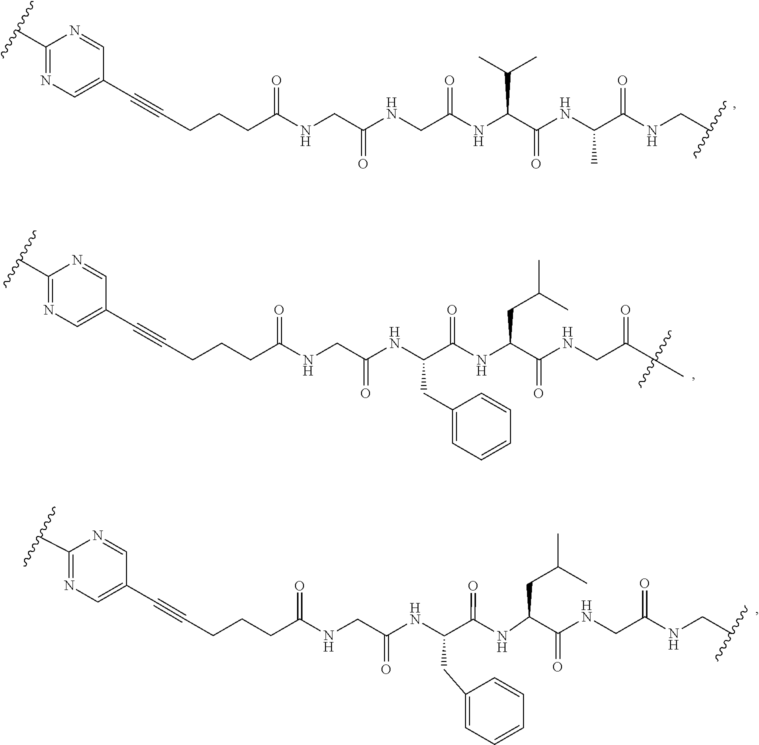 [SMILES: *CNC(=O)[C@H](C)NC(=O)[C@@H](NC(=O)CNC(=O)CNC(=O)CCCC#Cc1cnc(*)nc1)C(C)C ; *C(=O)CNC(=O)[C@H](CC(C)C)NC(=O)[C@H](Cc1ccccc1)NC(=O)CNC(=O)CCCC#Cc1cnc(*)nc1 ; *CNC(=O)CNC(=O)[C@H](CC(C)C)NC(=O)[C@H](Cc1ccccc1)NC(=O)CNC(=O)CCCC#Cc1cnc(*)nc1]

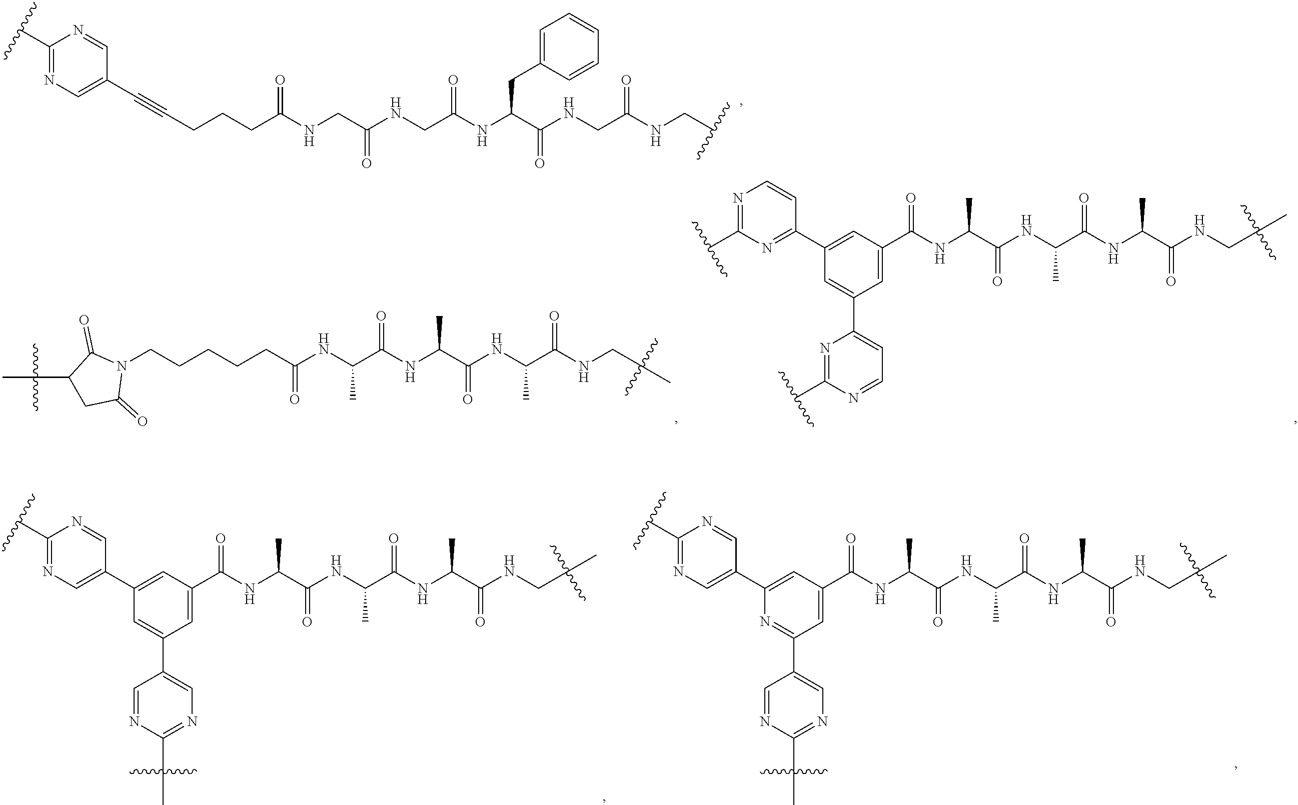 [SMILES: *CNC(=O)CNC(=O)[C@H](Cc1ccccc1)NC(=O)CNC(=O)CNC(=O)CCCC#Cc1cnc(*)nc1 ; *CNC(=O)[C@H](C)NC(=O)[C@H](C)NC(=O)[C@H](C)NC(=O)CCCCCN1C(=O)CC(*)C1=O ; *CNC(=O)[C@H](C)NC(=O)[C@H](C)NC(=O)[C@H](C)NC(=O)c1cc(-c2ccnc(*)n2)cc(-c2ccnc(*)n2)c1 ; *CNC(=O)[C@H](C)NC(=O)[C@H](C)NC(=O)[C@H](C)NC(=O)c1cc(-c2cnc(*)nc2)cc(-c2cnc(*)nc2)c1 ; *CNC(=O)[C@H](C)NC(=O)[C@H](C)NC(=O)[C@H](C)NC(=O)c1cc(-c2cnc(*)nc2)nc(-c2cnc(*)nc2)c1]

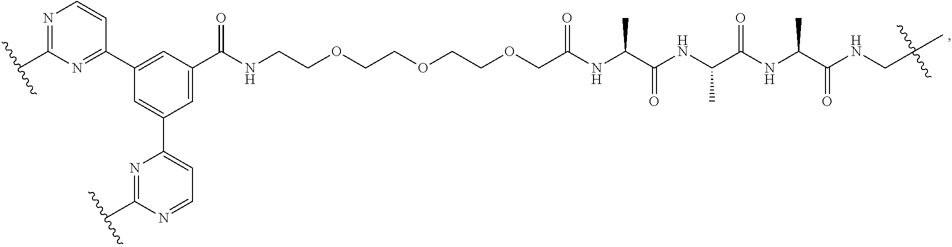 [SMILES: *CNC(=O)[C@H](C)NC(=O)[C@H](C)NC(=O)[C@H](C)NC(=O)COCCOCCOCCNC(=O)c1cc(-c2ccnc(*)n2)cc(-c2ccnc(*)n2)c1]

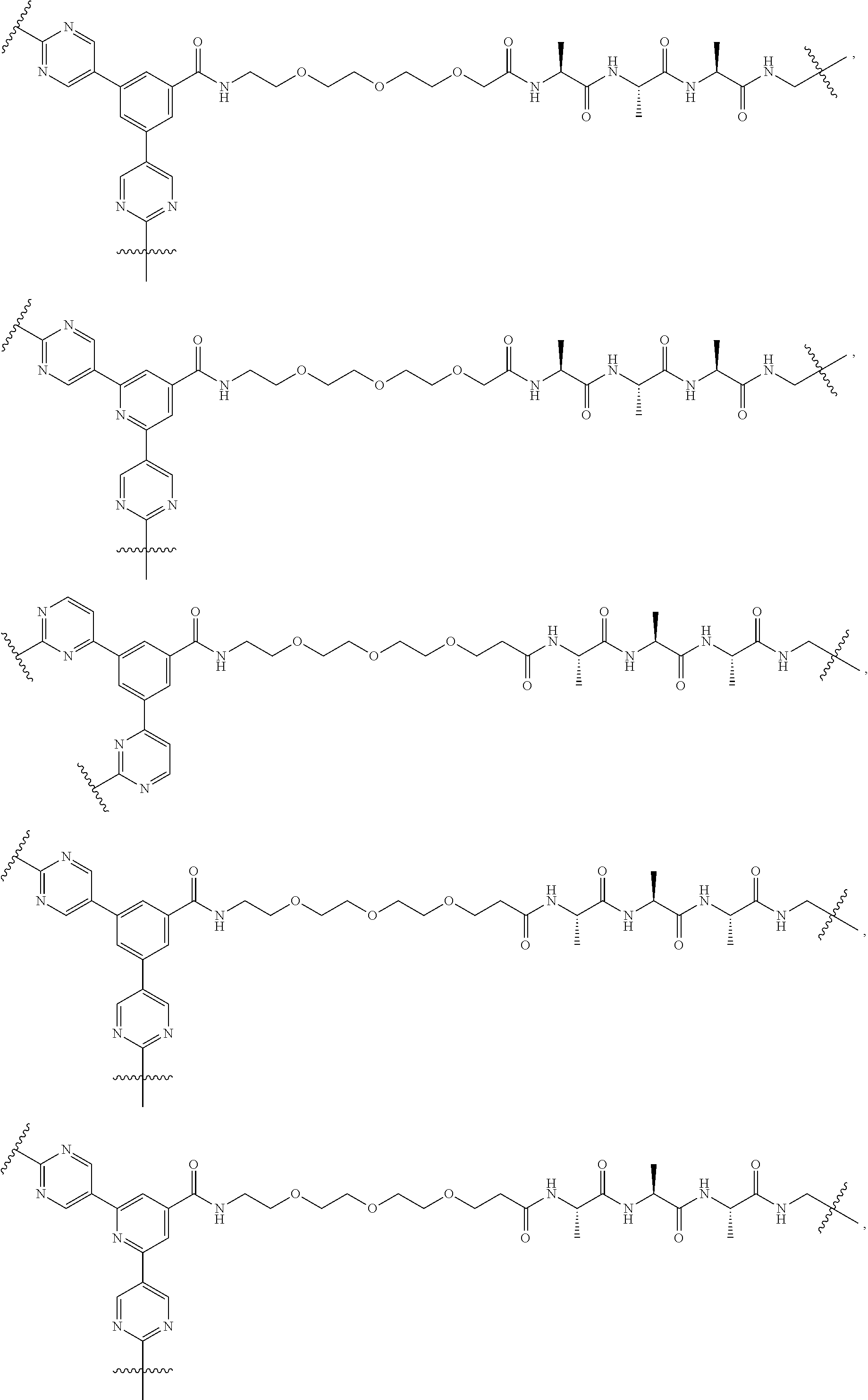

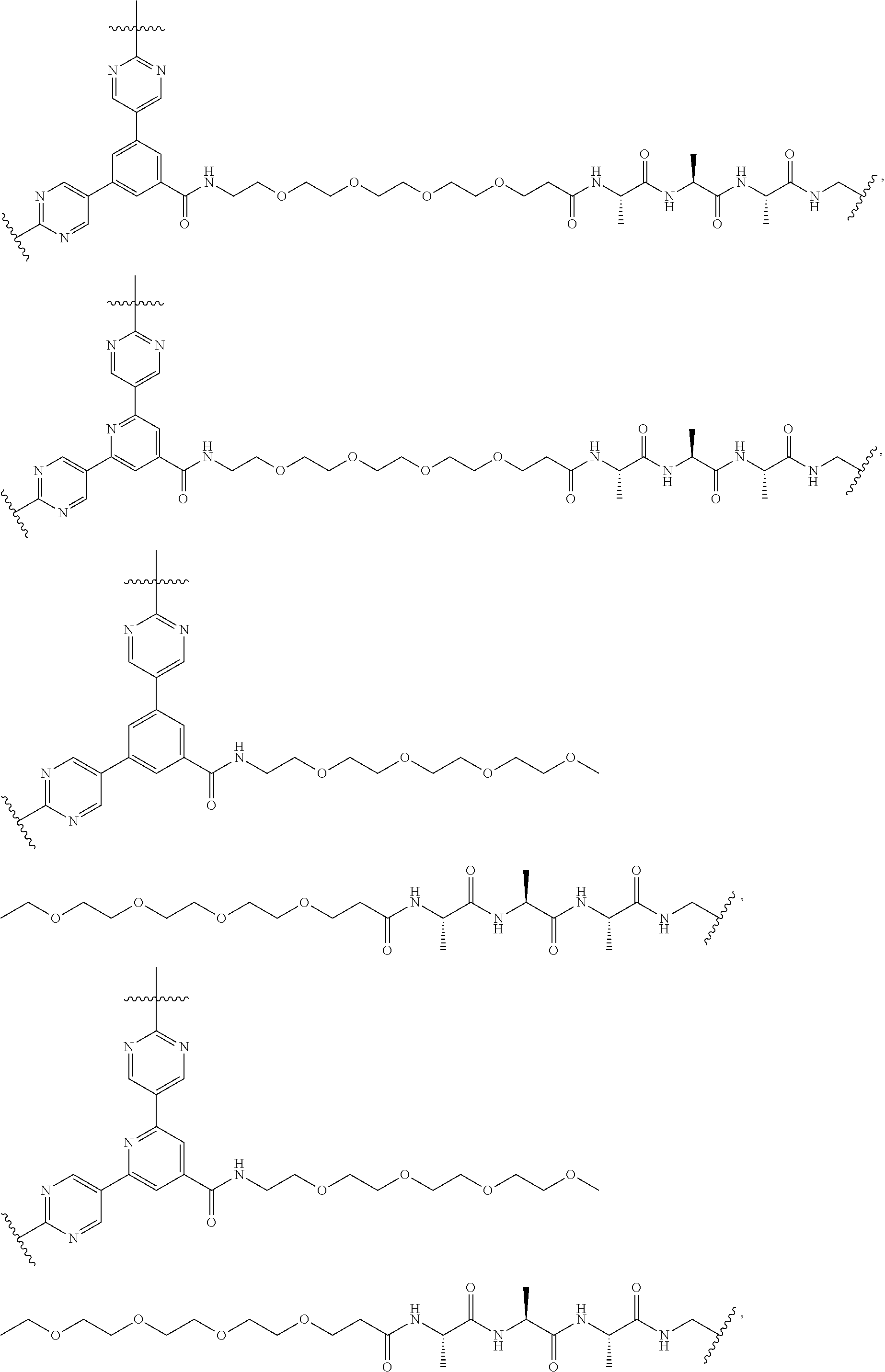

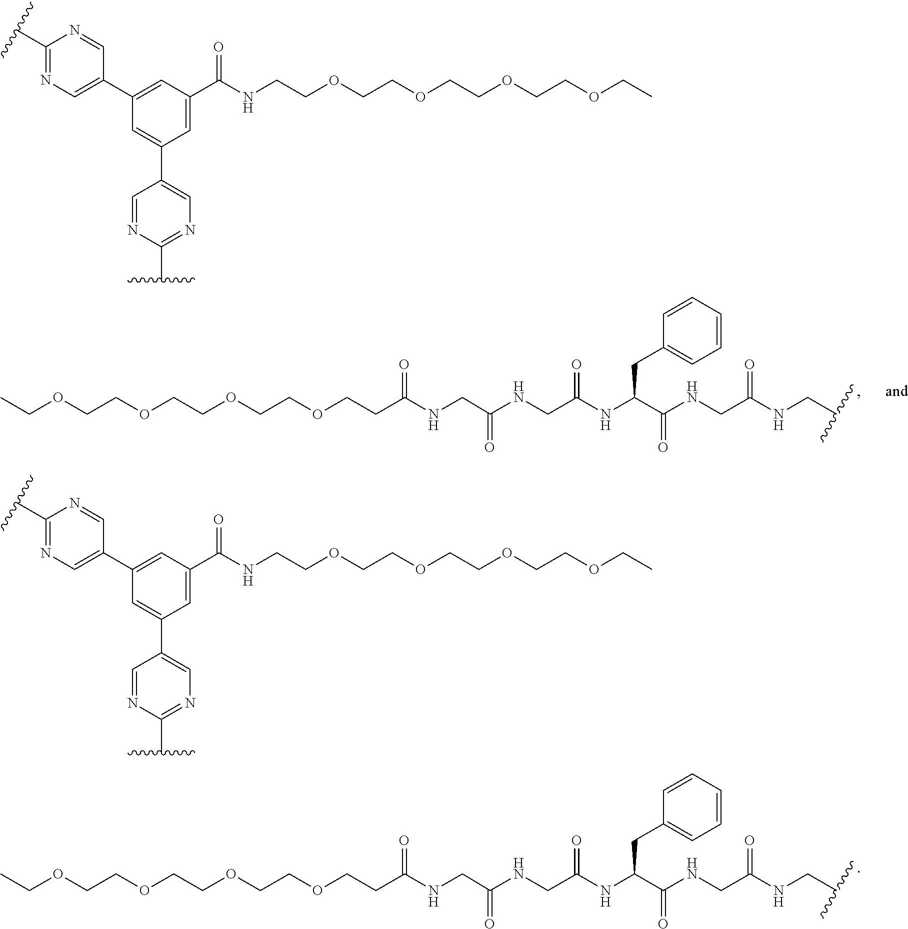

In a further embodiment of the antibody-drug conjugate,

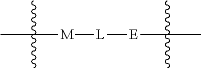

is selected from the following structures:

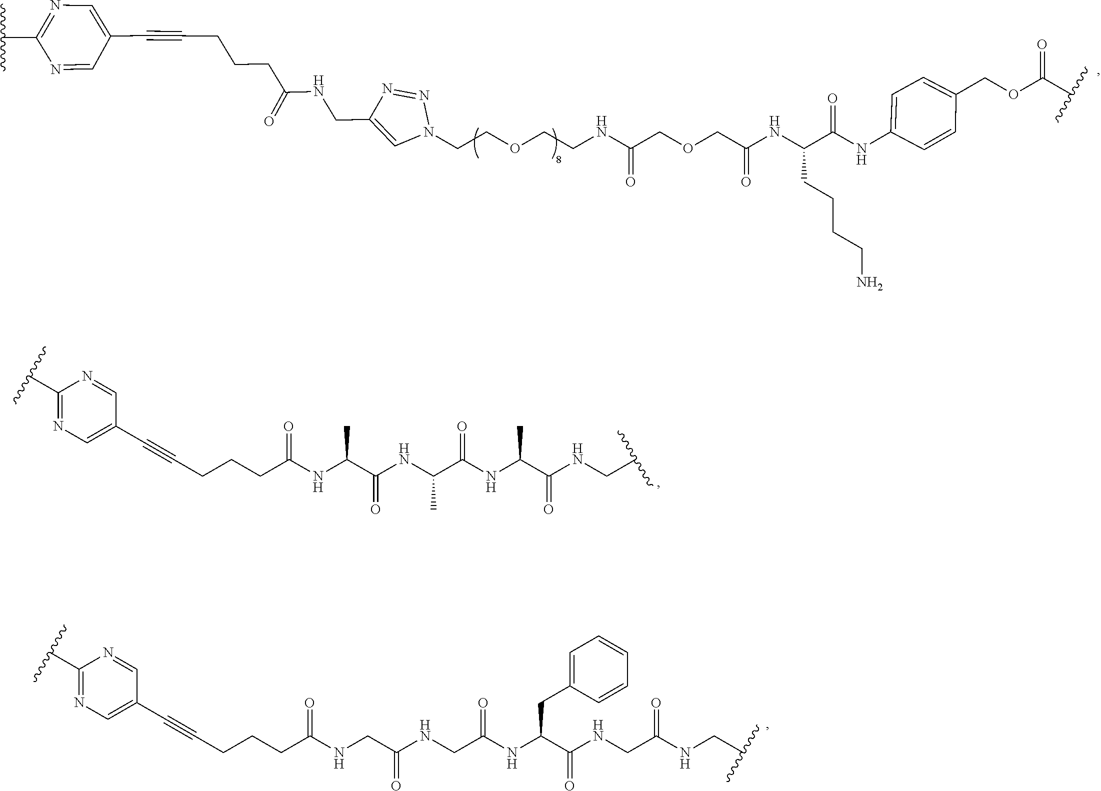

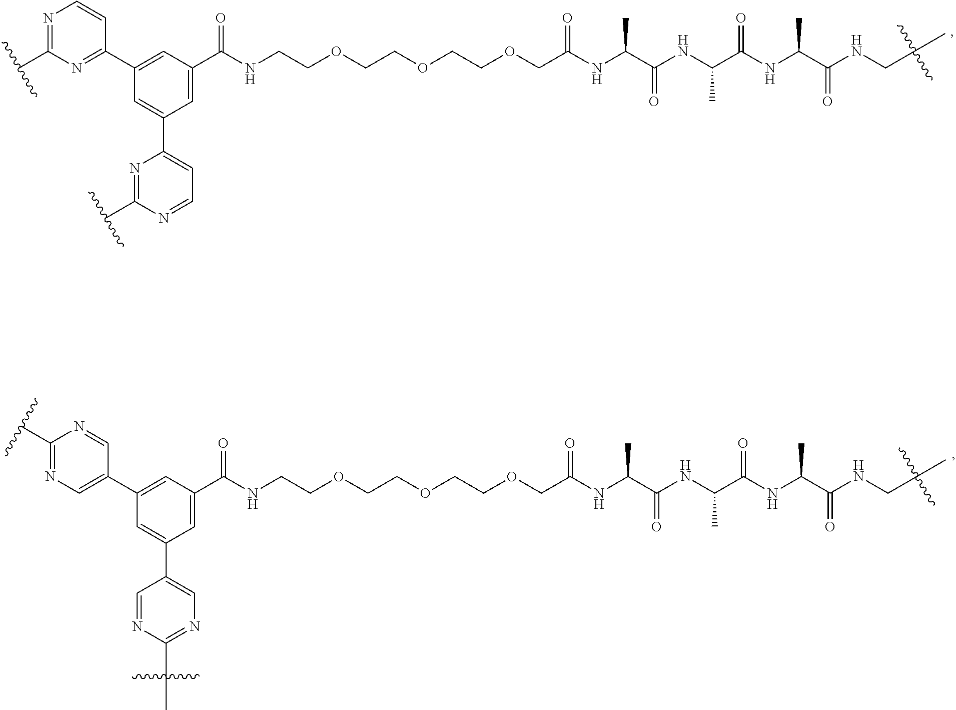 [SMILES: *CNC(=O)[C@H](C)NC(=O)[C@H](C)NC(=O)[C@H](C)NC(=O)COCCOCCOCCNC(=O)c1cc(-c2ccnc(*)n2)cc(-c2ccnc(*)n2)c1 ; *CNC(=O)[C@H](C)NC(=O)[C@H](C)NC(=O)[C@H](C)NC(=O)COCCOCCOCCNC(=O)c1cc(-c2cnc(*)nc2)cc(-c2cnc(*)nc2)c1]

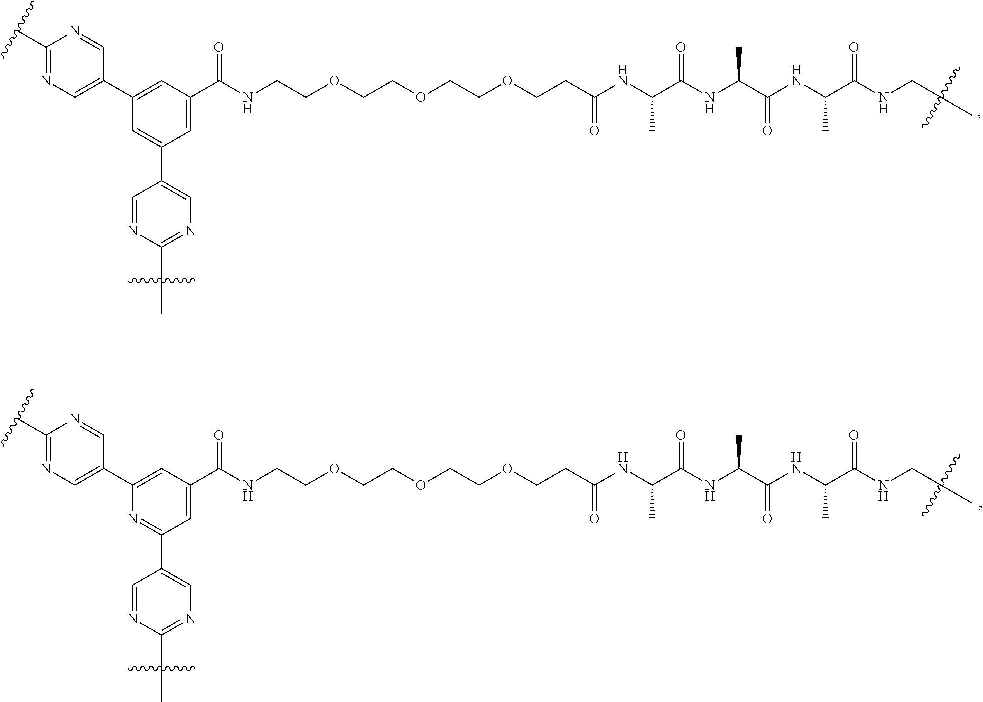 [SMILES: *CNC(=O)[C@H](C)NC(=O)[C@H](C)NC(=O)[C@H](C)NC(=O)CCOCCOCCOCCNC(=O)c1cc(-c2cnc(*)nc2)cc(-c2cnc(*)nc2)c1 ; *CNC(=O)[C@H](C)NC(=O)[C@H](C)NC(=O)[C@H](C)NC(=O)CCOCCOCCOCCNC(=O)c1cc(-c2cnc(*)nc2)nc(-c2cnc(*)nc2)c1]

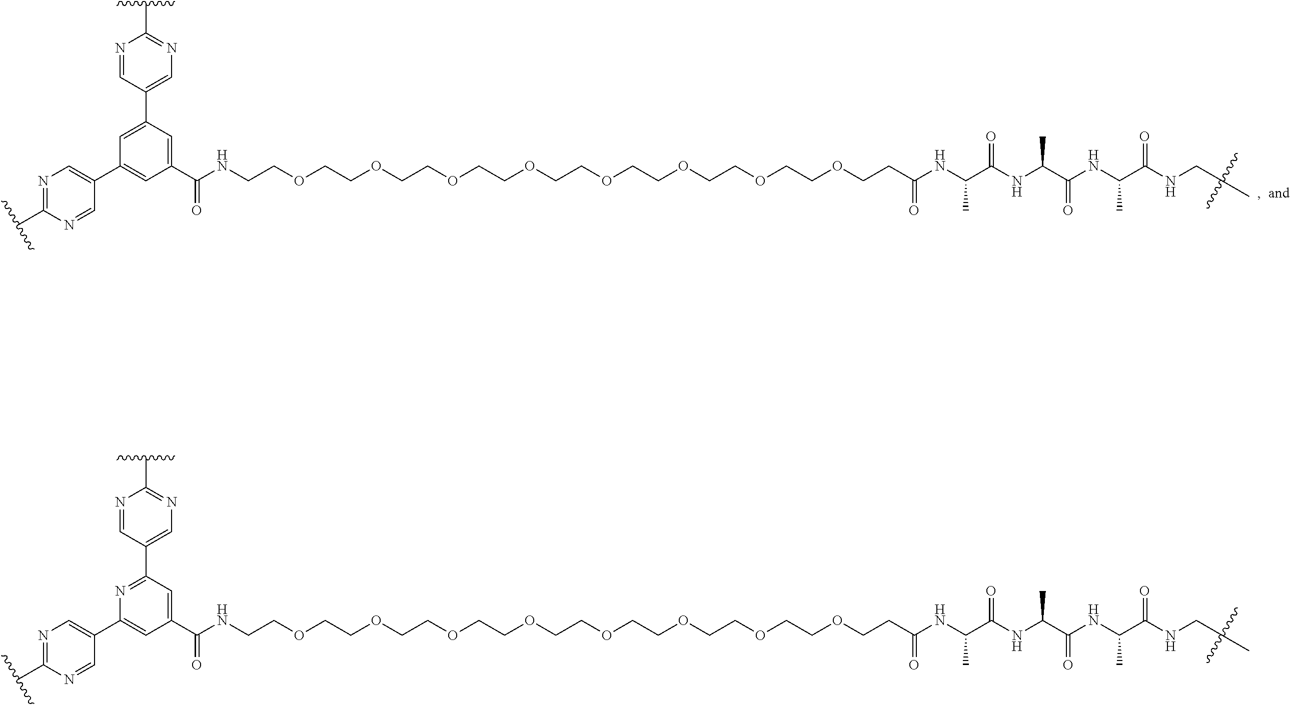 [SMILES: *CNC(=O)[C@H](C)NC(=O)[C@H](C)NC(=O)[C@H](C)NC(=O)CCOCCOCCOCCOCCOCCOCCOCCOCCNC(=O)c1cc(-c2cnc(*)nc2)cc(-c2cnc(*)nc2)c1 ; *CNC(=O)[C@H](C)NC(=O)[C@H](C)NC(=O)[C@H](C)NC(=O)CCOCCOCCOCCOCCOCCOCCOCCOCCNC(=O)c1cc(-c2cnc(*)nc2)nc(-c2cnc(*)nc2)c1]

In a further embodiment of the antibody-drug conjugate, the cytotoxic drug is selected from the group consisting of tubulin inhibitors, DNA intercalating agents, DNA Topoisomerase inhibitors, and RNA polymerase inhibitors.

In a further embodiment of the antibody-drug conjugate, the tubulin inhibitor is an auristatin compound or a maytansinoid compound.

In a further embodiment of the antibody-drug conjugate, the DNA intercalator is pyrrolobenzodiazepine (PBD).

In a further embodiment of the antibody-drug conjugate, the DNA topoisomerase inhibitor is a topoisomerase I inhibitor or a topoisomerase II inhibitor.

In a further embodiment of the antibody-drug conjugate, the topoisomerase I inhibitor is selected from camptothecin, hydroxycamptothecin, 9-aminocamptothecin, SN-38, irinotecan, topotecan, bellotecan, rubotecan, and a pharmaceutically acceptable salt, ester, or analog thereof, and the topoisomerase II inhibitor is selected from doxorubicin, PNU-159682, docarmicin, daunorubicin, mitoxantrone, podophyllin toxin, etoposide, and a pharmaceutically acceptable salt, ester, or analog thereof.

In a further embodiment of the antibody-drug conjugate, the RNA polymerase inhibitor is α-amanitin or a pharmaceutically acceptable salt, ester, or analog thereof.

In a further embodiment of the antibody-drug conjugate, the cytotoxic drug is selected from the group consisting of compounds represented by Formula I and Formula II, or pharmaceutically acceptable salts, esters, stereoisomers, tautomers or precursors of the compounds represented by Formula I and Formula II:

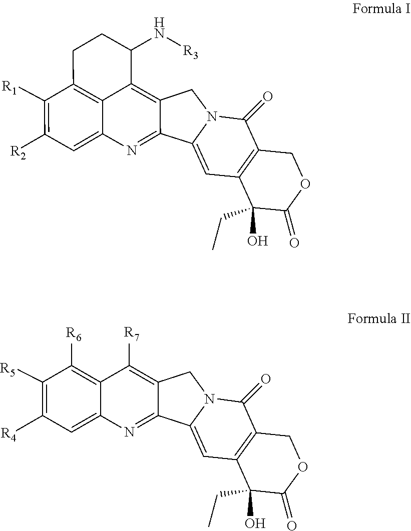

wherein R₁ and R₂ are each independently selected from C₁₋₆ Alkyl and halogen;
R₃ is selected from H and —CO—CH₂OH;
R₄ and R₅ are each independently selected from H, halogen, and hydroxyl, or R₄ and R₅ are connected to form a 5-6 membered oxygen-containing heterocyclic ring;
R₆ is selected from hydrogen or —C₁₋₄ alkylene-NRₐR_{b}; and
R₇ is selected from C₁₋₆ alkyl, —C₁₋₄ alkylene-NRₐR_{b}, —C₁₋₄ alkylene-SiRₐR_{b}R_{c}, —SiRₐR_{b}R_{c}, —C₁₋₄ alkylene=N—ORₐ; wherein, Rₐ, R_{b} and R_{c} are independently selected from H, C₁₋₆ alkyl, —SO₂—C₁₋₆ alkyl, and —CO—C₁₋₆ alkyl at each occurrence; wherein optionally Rₐ and R_{b} connected to the associated atoms form a 5-6 membered nitrogen containing heterocyclic ring.

In a further embodiment of the antibody-drug conjugate, the cytotoxic drug is selected from the following compounds or pharmaceutically acceptable salts, esters, stereoisomers, tautomers, or prodrugs of said compounds:

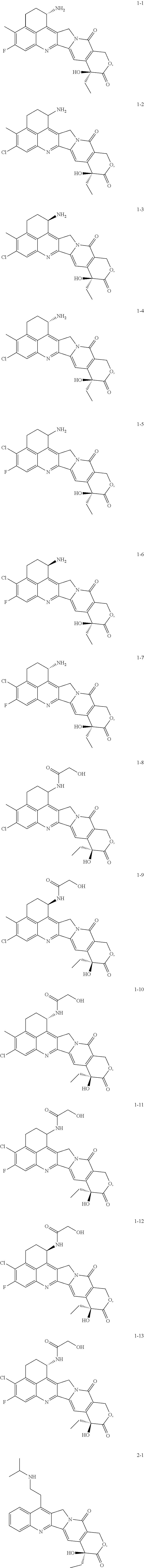

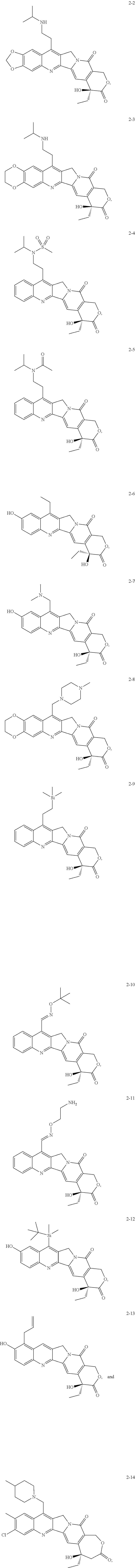

wherein the corresponding residue of the cytotoxic drug obtained after connecting the cytotoxic drug to the linker is D in the formula described above.

In a further embodiment of the antibody-drug conjugate, the cytotoxic drug is selected from the following compounds or pharmaceutically acceptable salts, esters, stereoisomers, tautomers or prodrugs of said compounds:

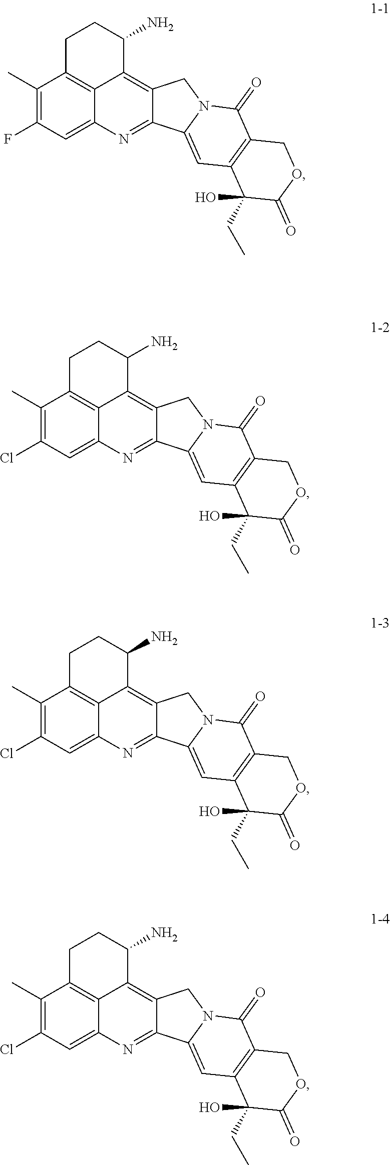 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3]

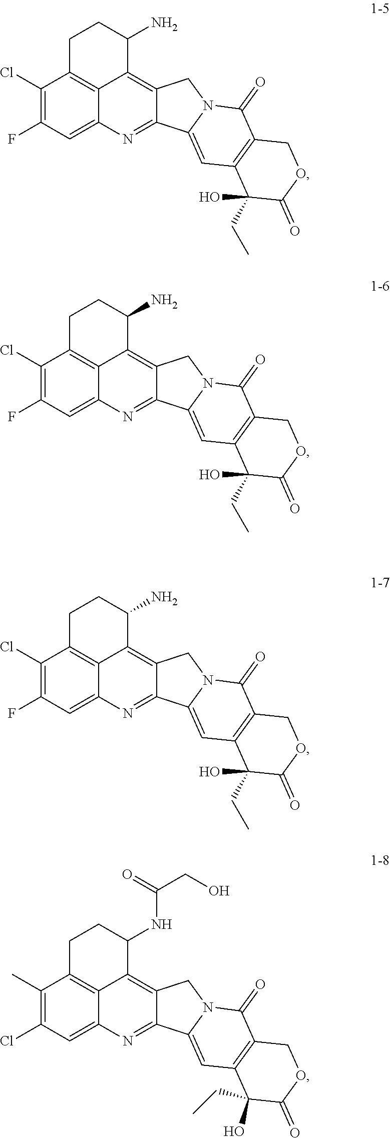 [SMILES: CC[C@@]1(O)C(=O)OCc2c1cc1n(c2=O)Cc2c-1nc1cc(F)c(Cl)c3c1c2C(N)CC3 ; CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3]

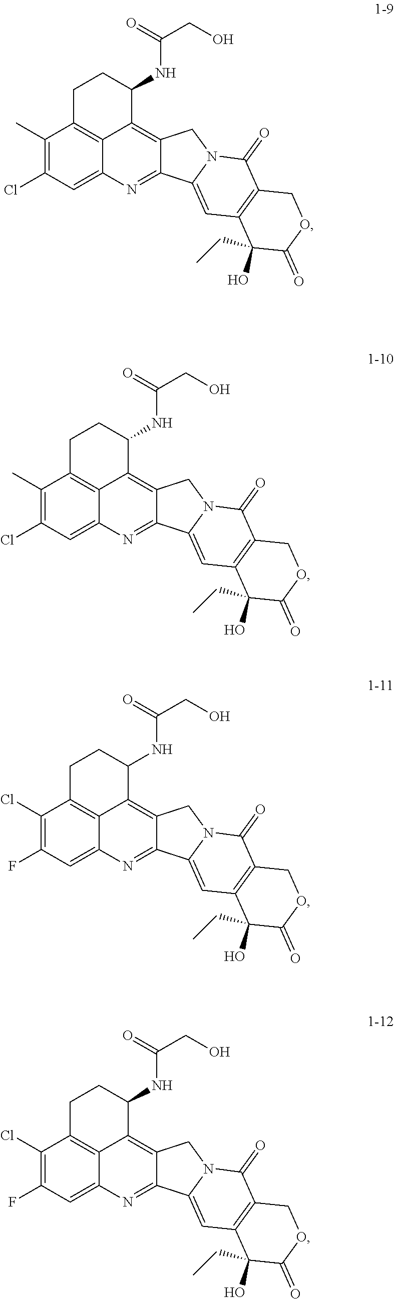 [SMILES: CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3]

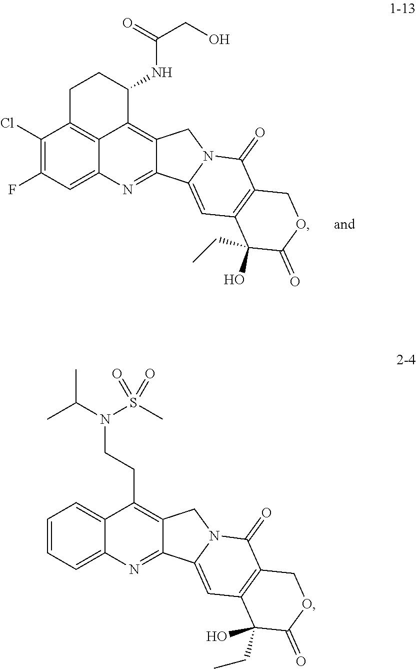 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1ccccc1c2CCN(C(C)C)S(C)(=O)=O]

wherein the corresponding residue of the cytotoxic drug obtained after connecting the cytotoxic drug to the linker is D in the formula described above.

In a further embodiment of the antibody-drug conjugate, D is a monovalent structure obtained by the loss of one H from an —OH, an —NH₂, or a secondary amine on the cytotoxic drug.

In a further embodiment of the antibody-drug conjugate, the antibody-drug conjugate is selected from:

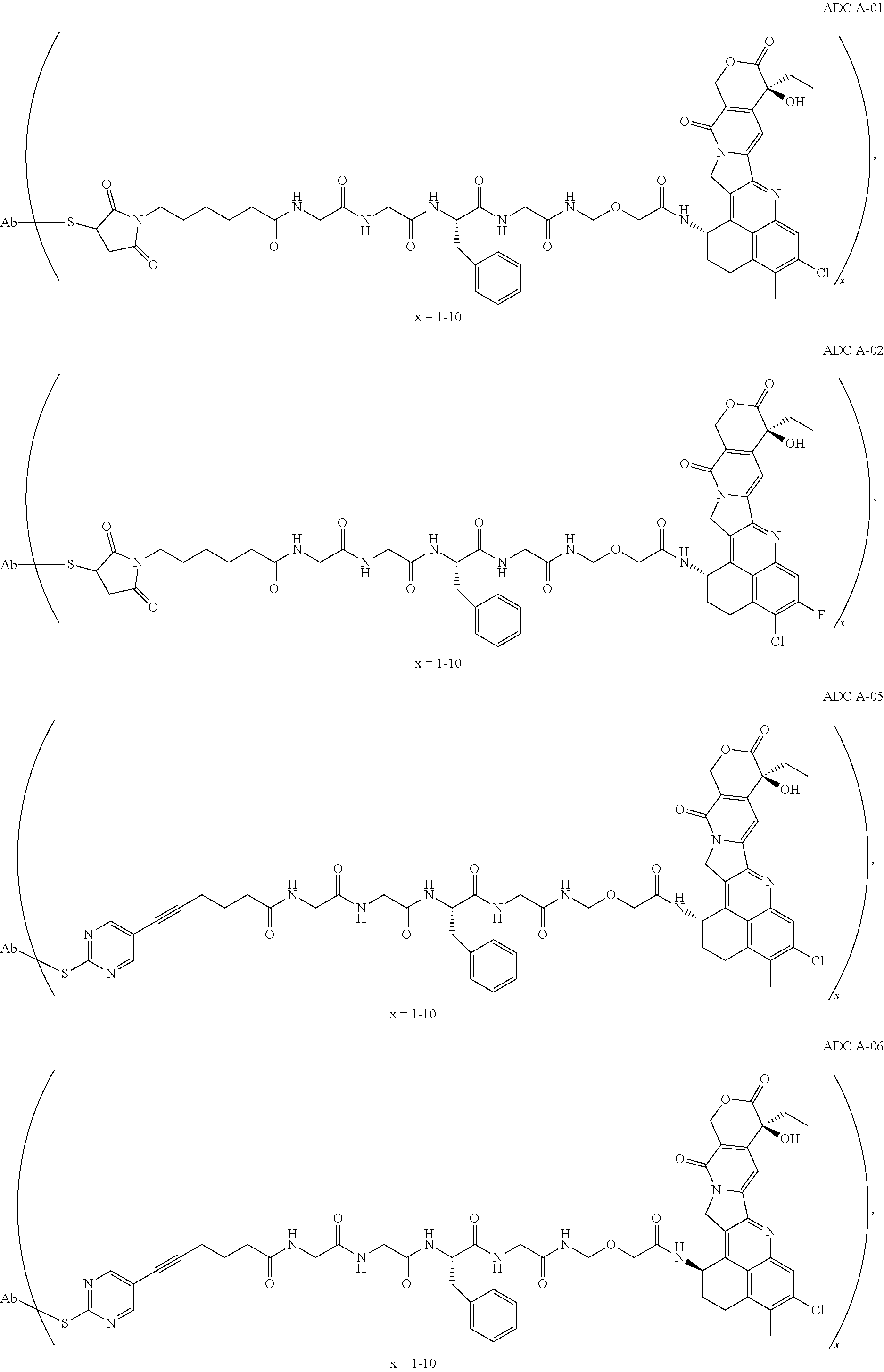

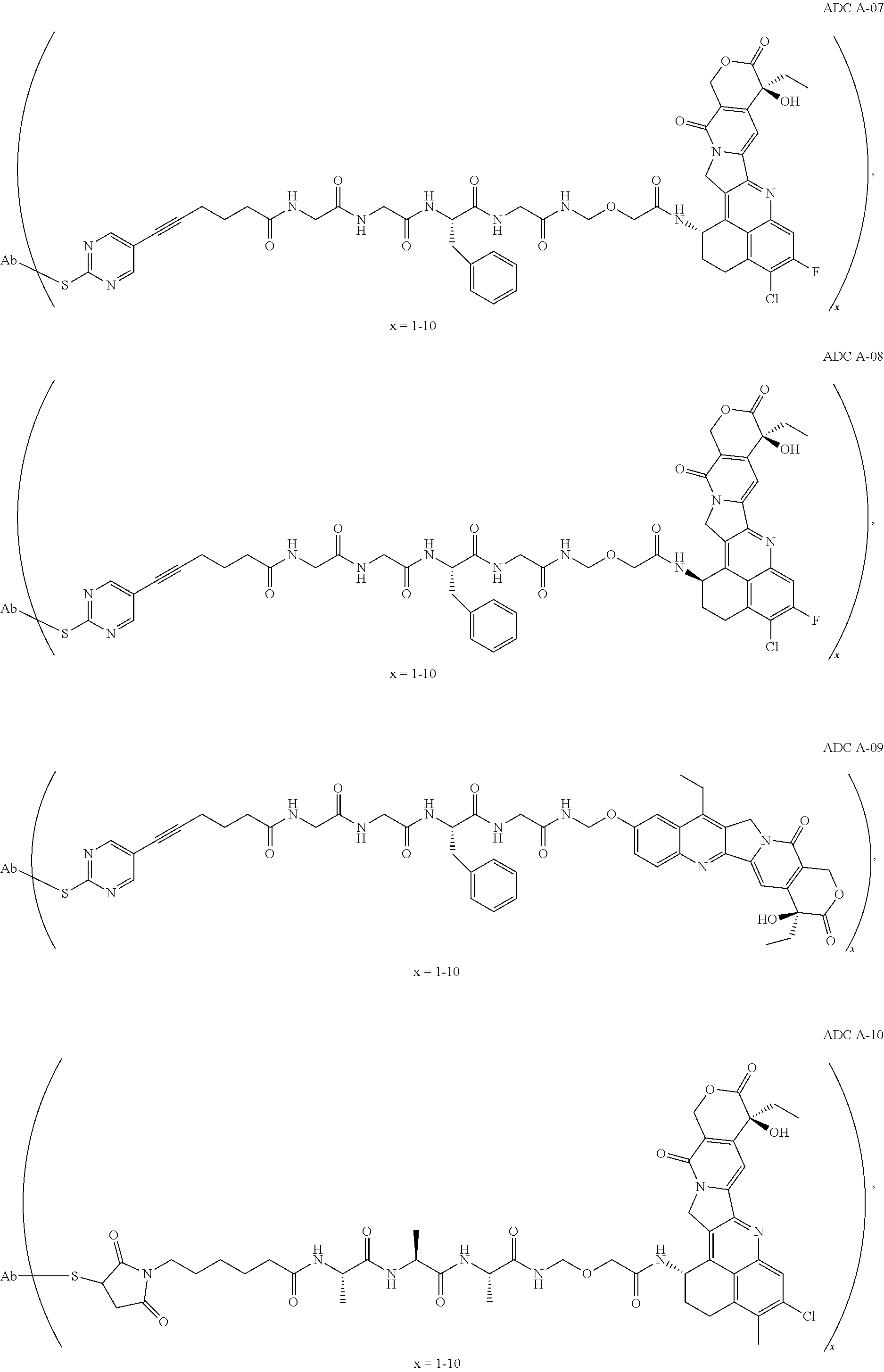

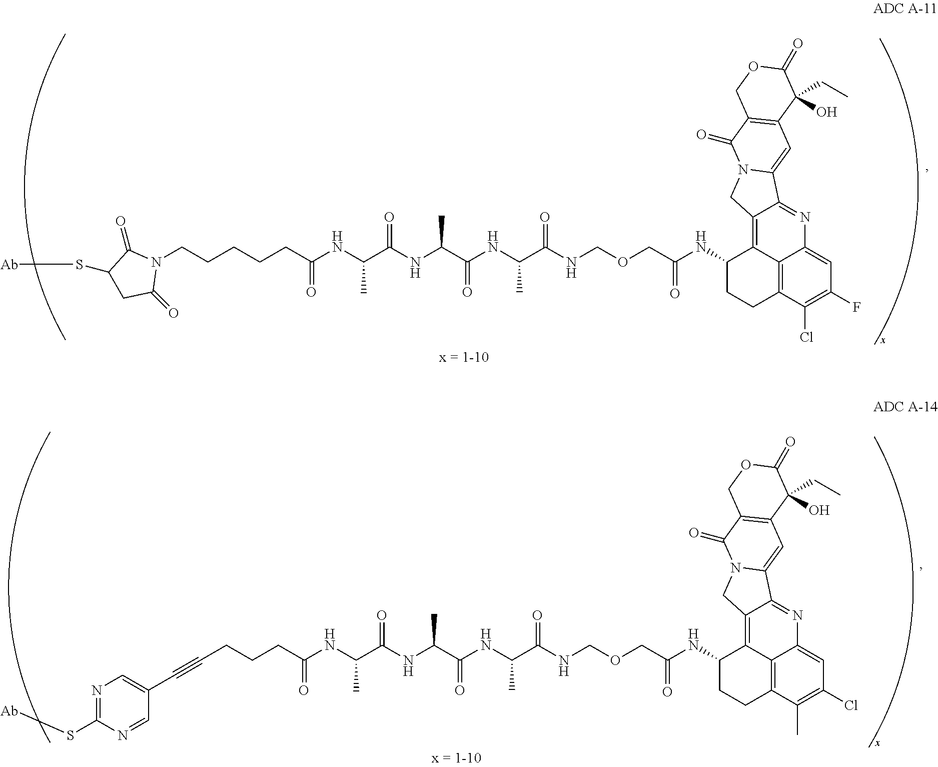

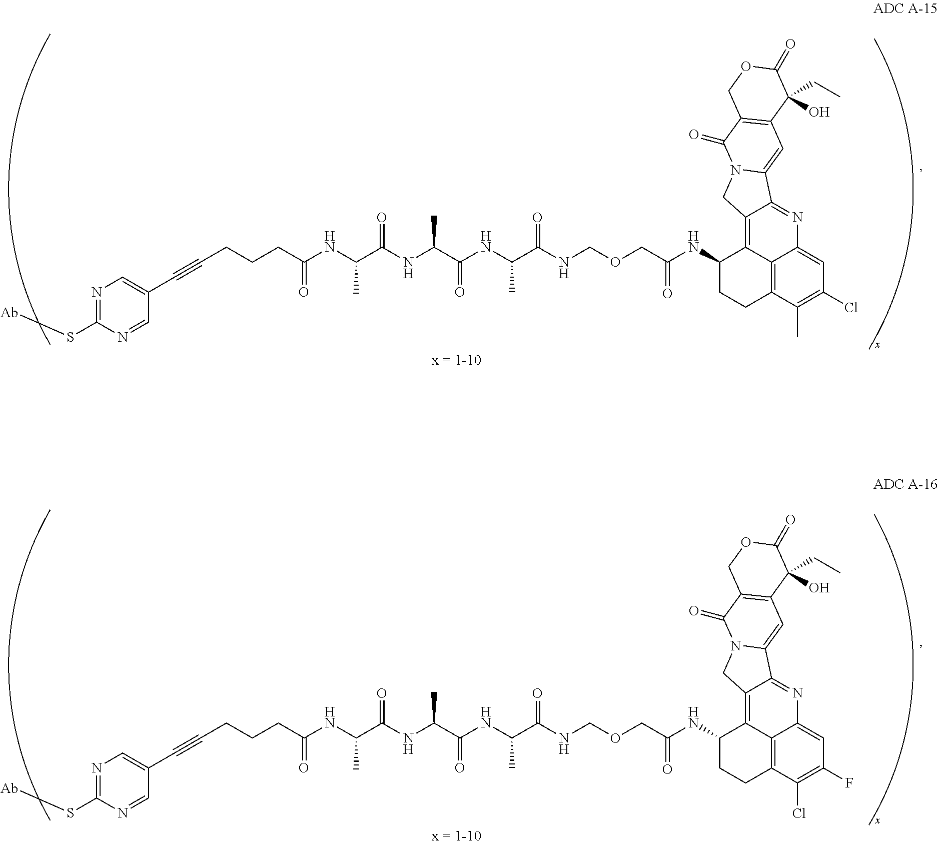

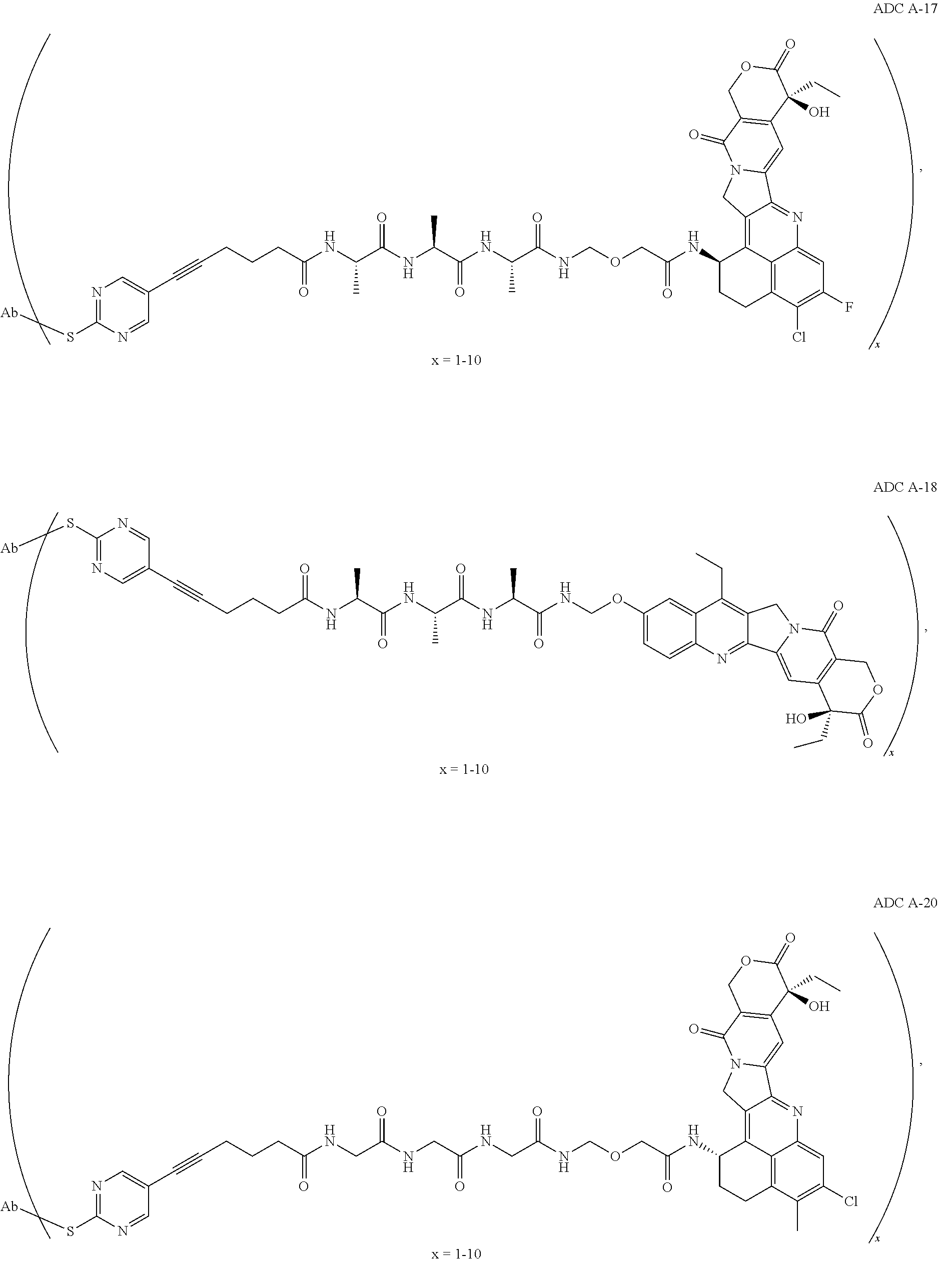

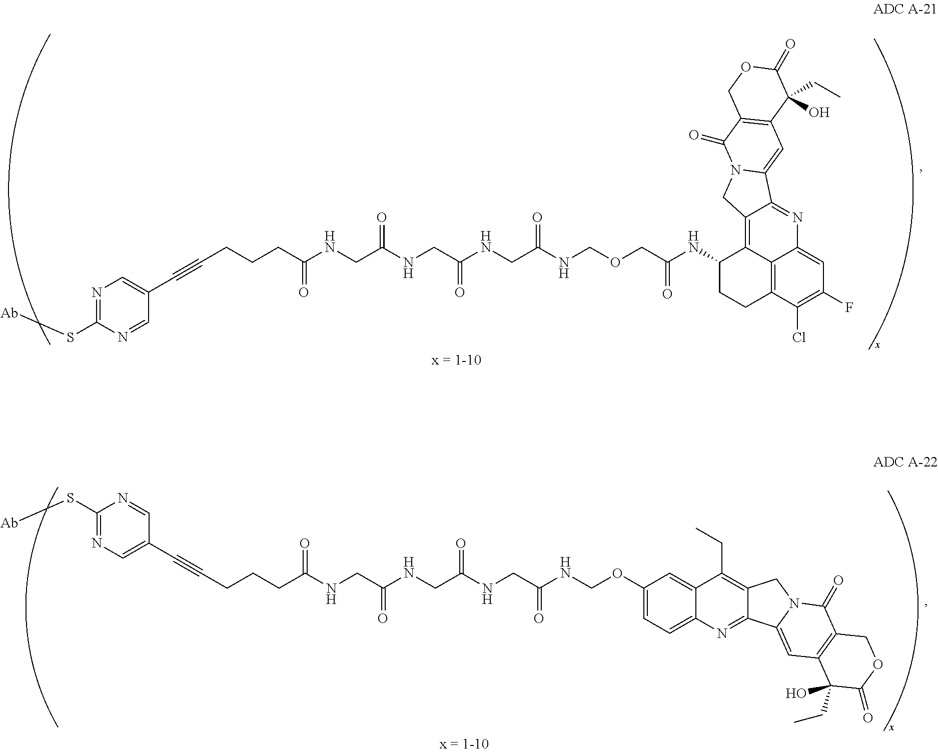

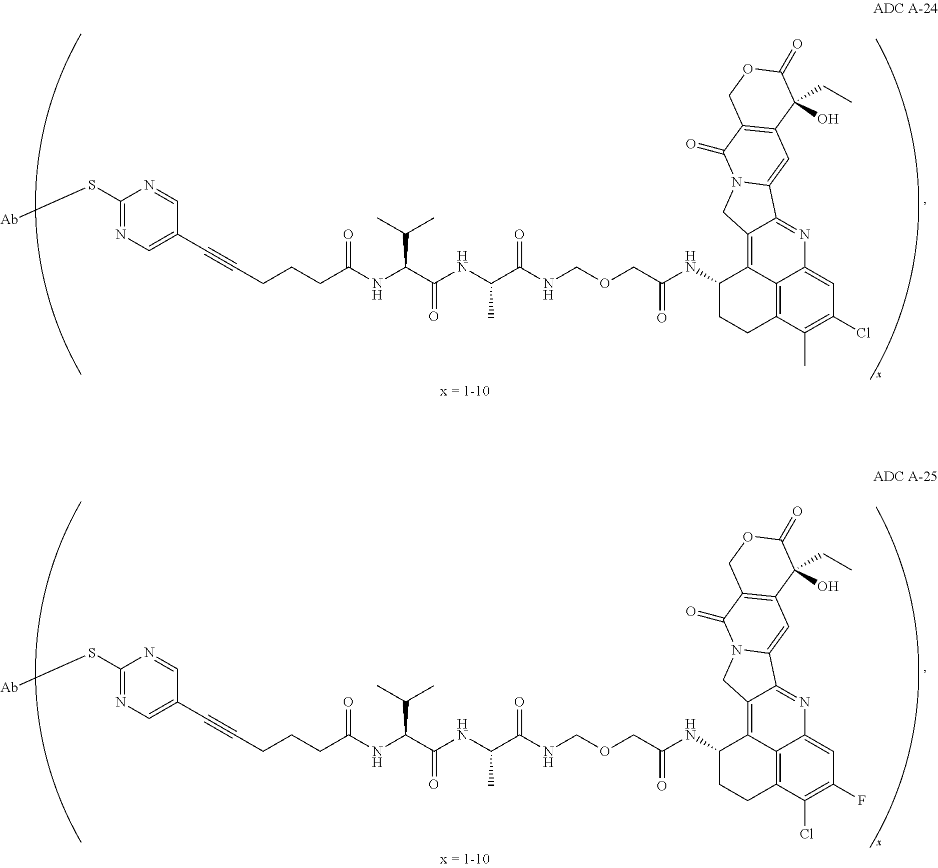

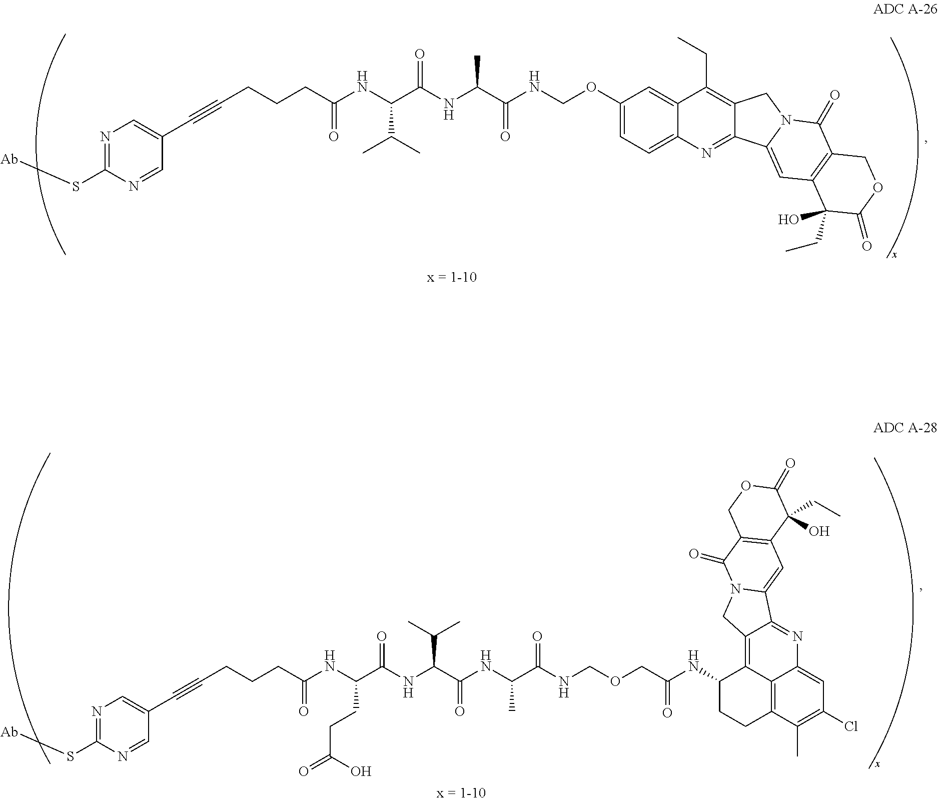

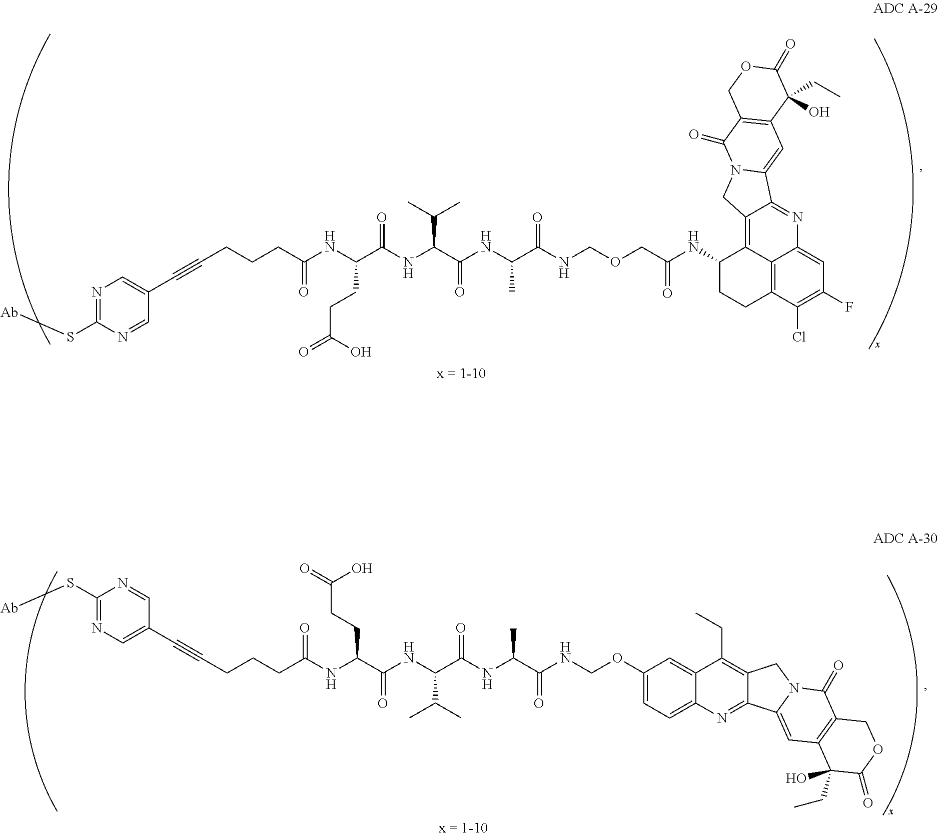

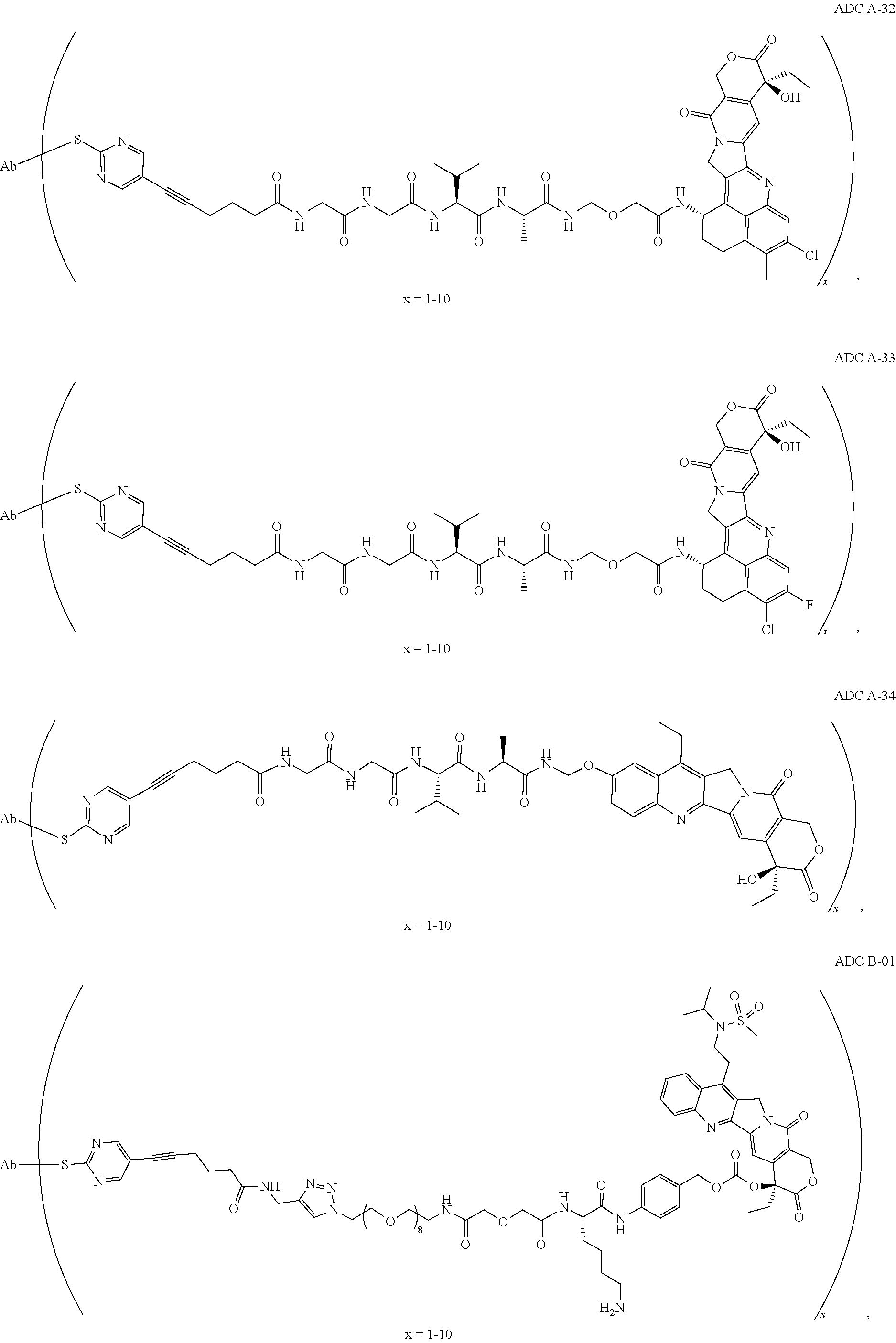

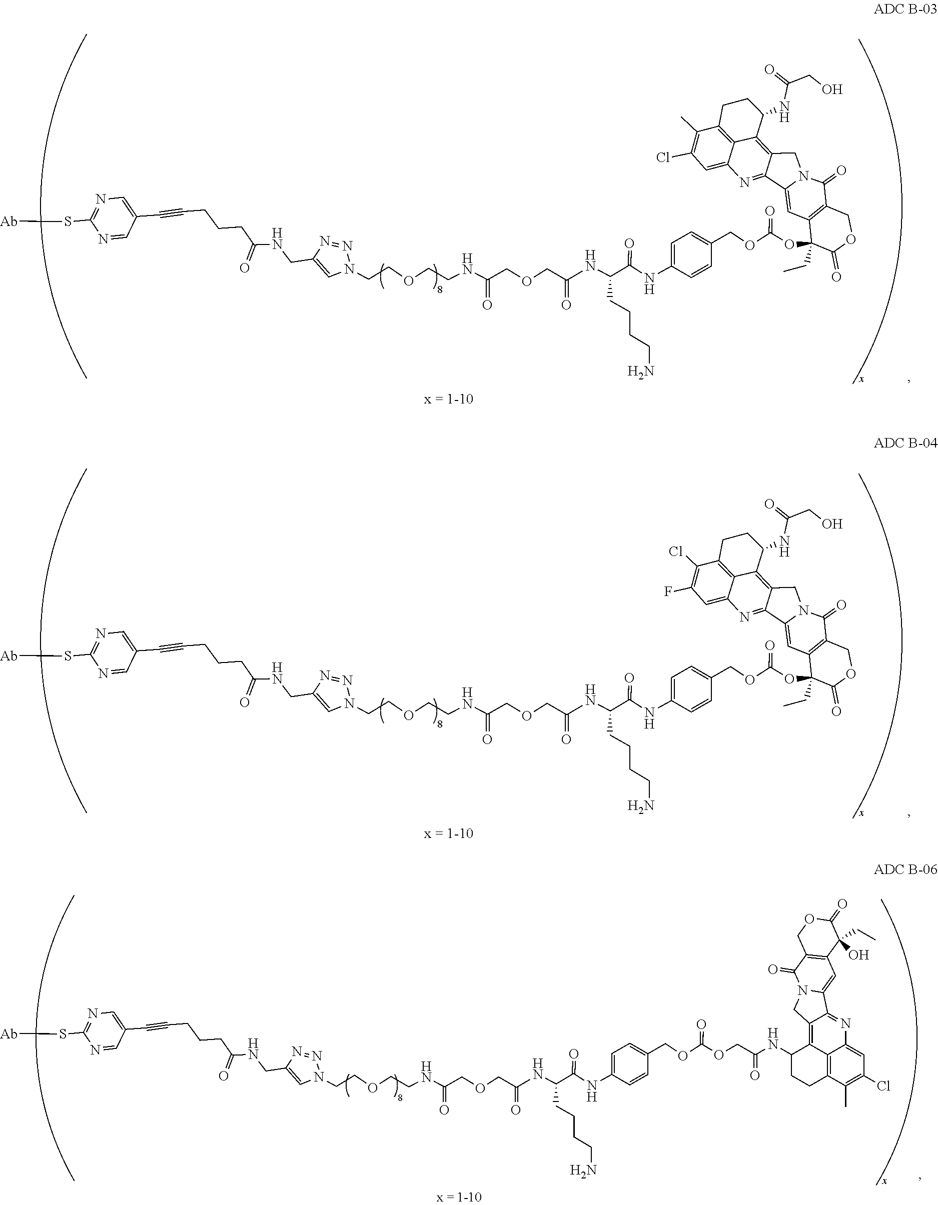

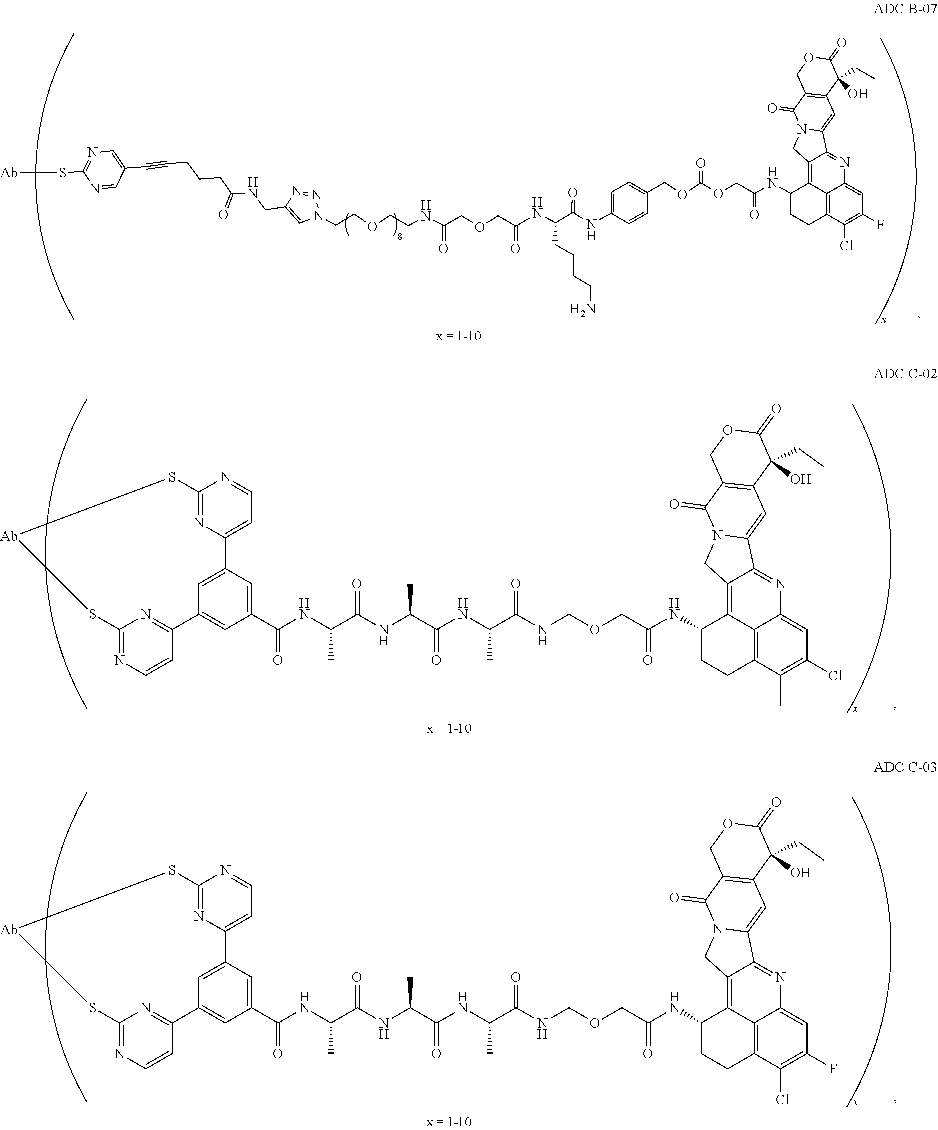

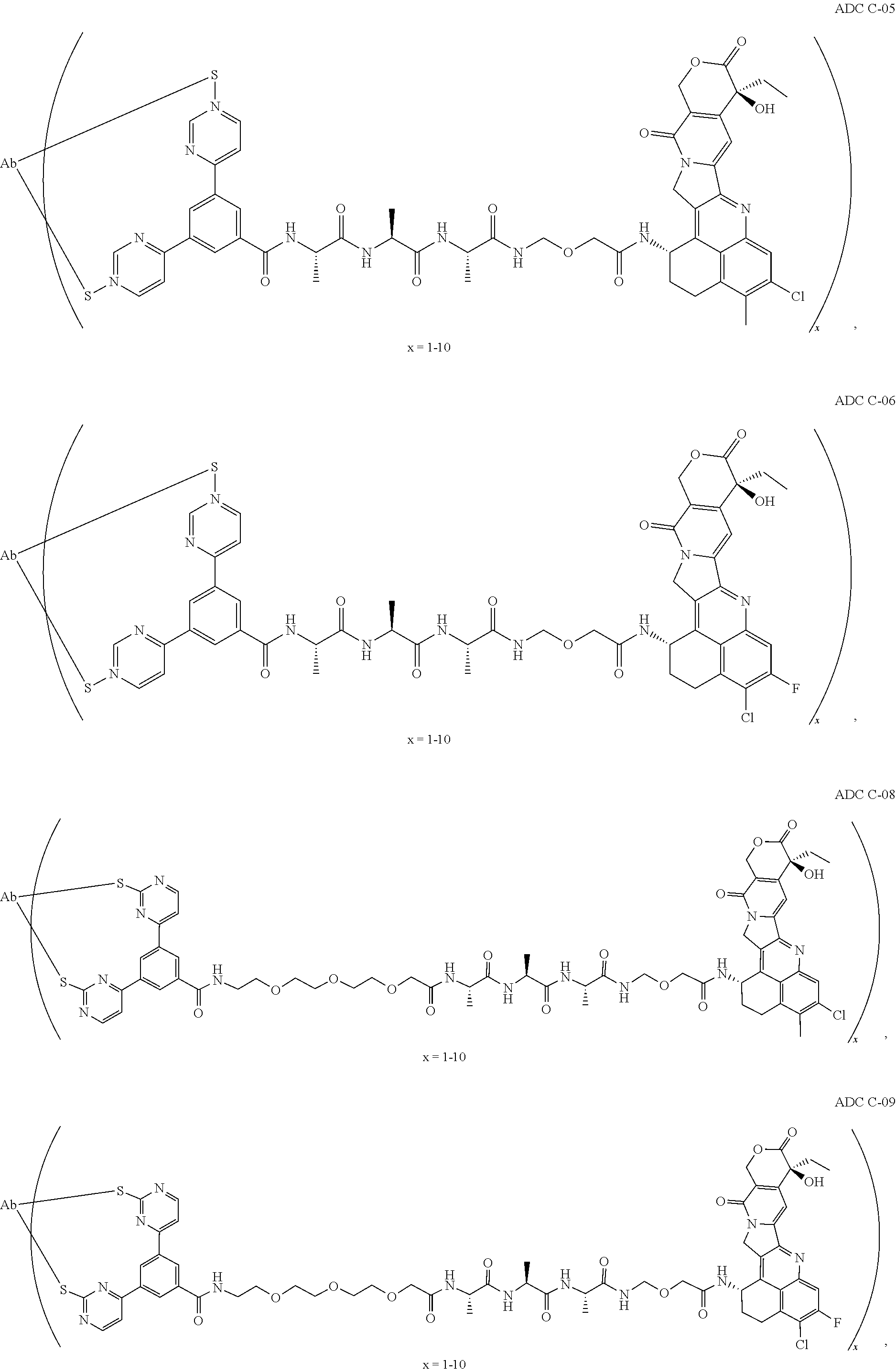

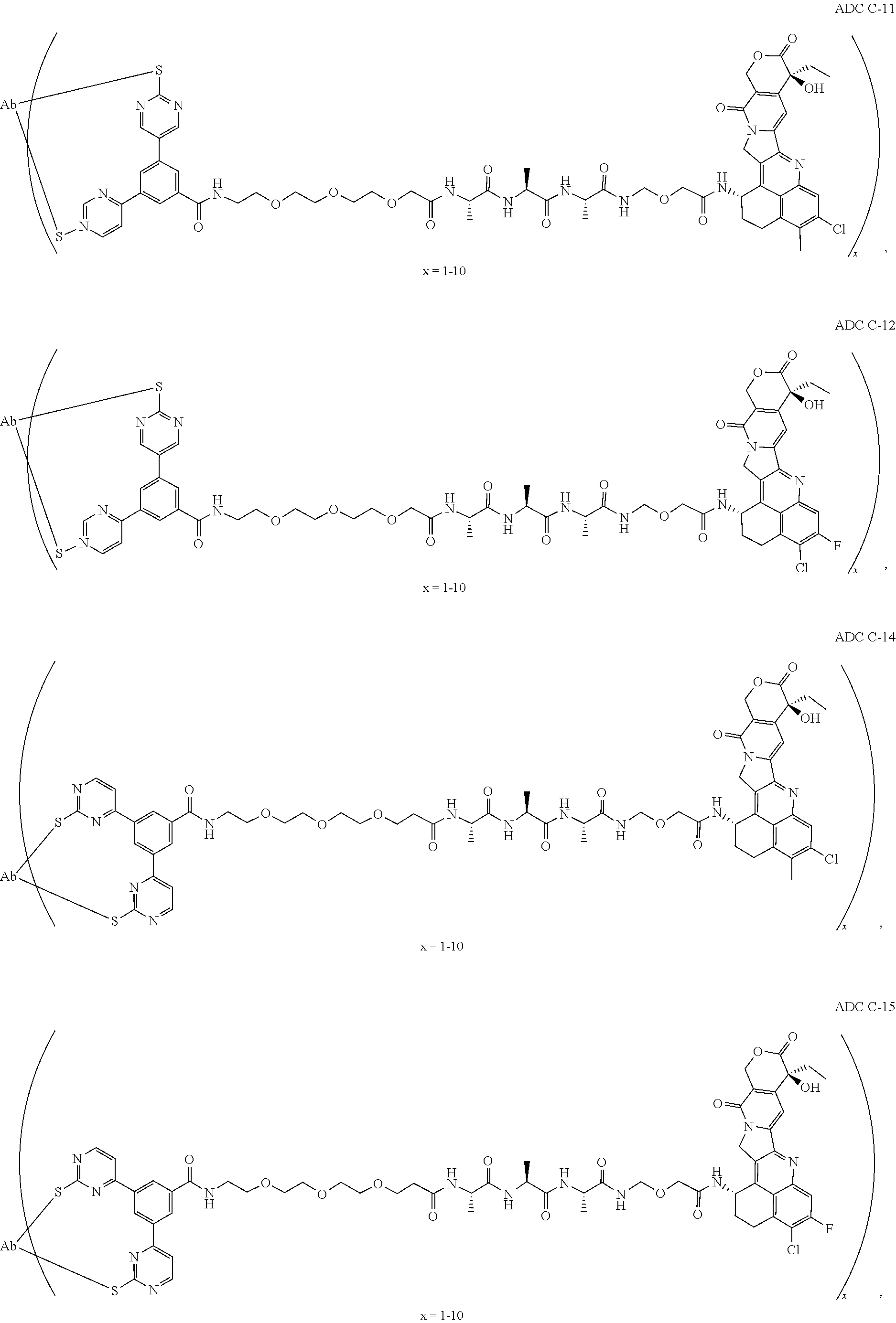

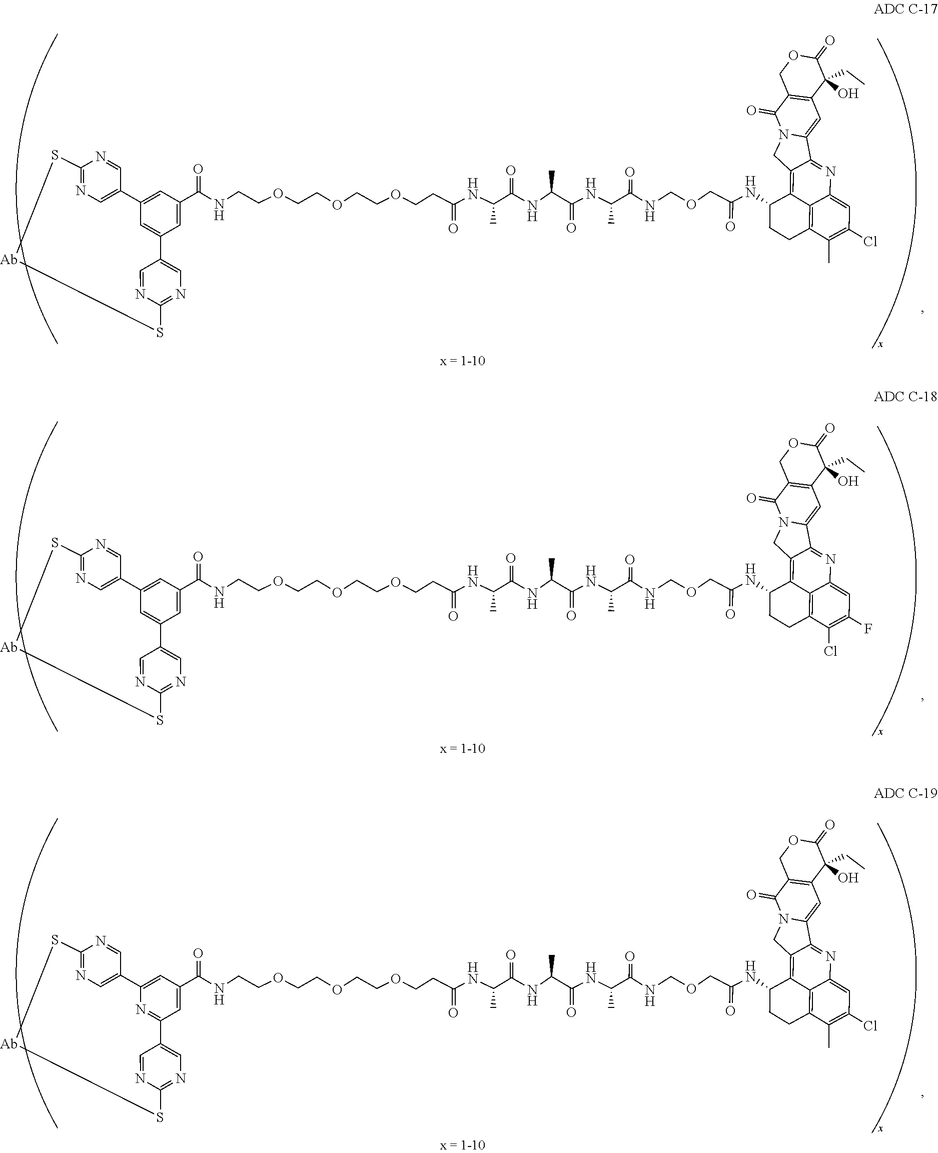

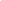

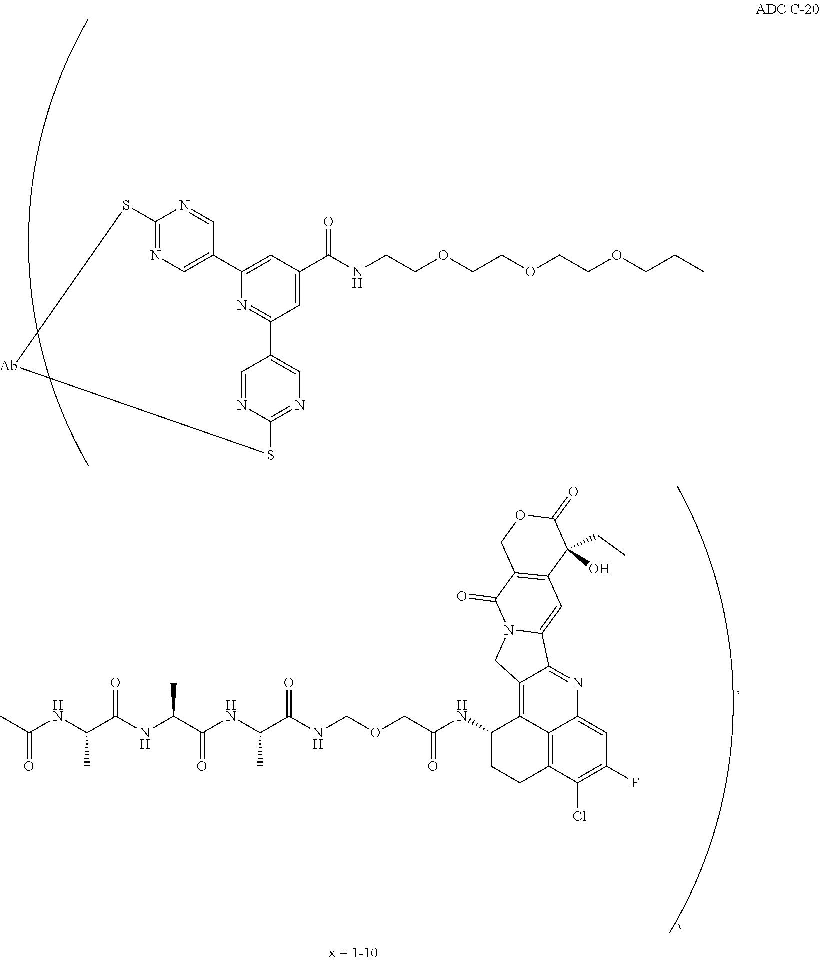

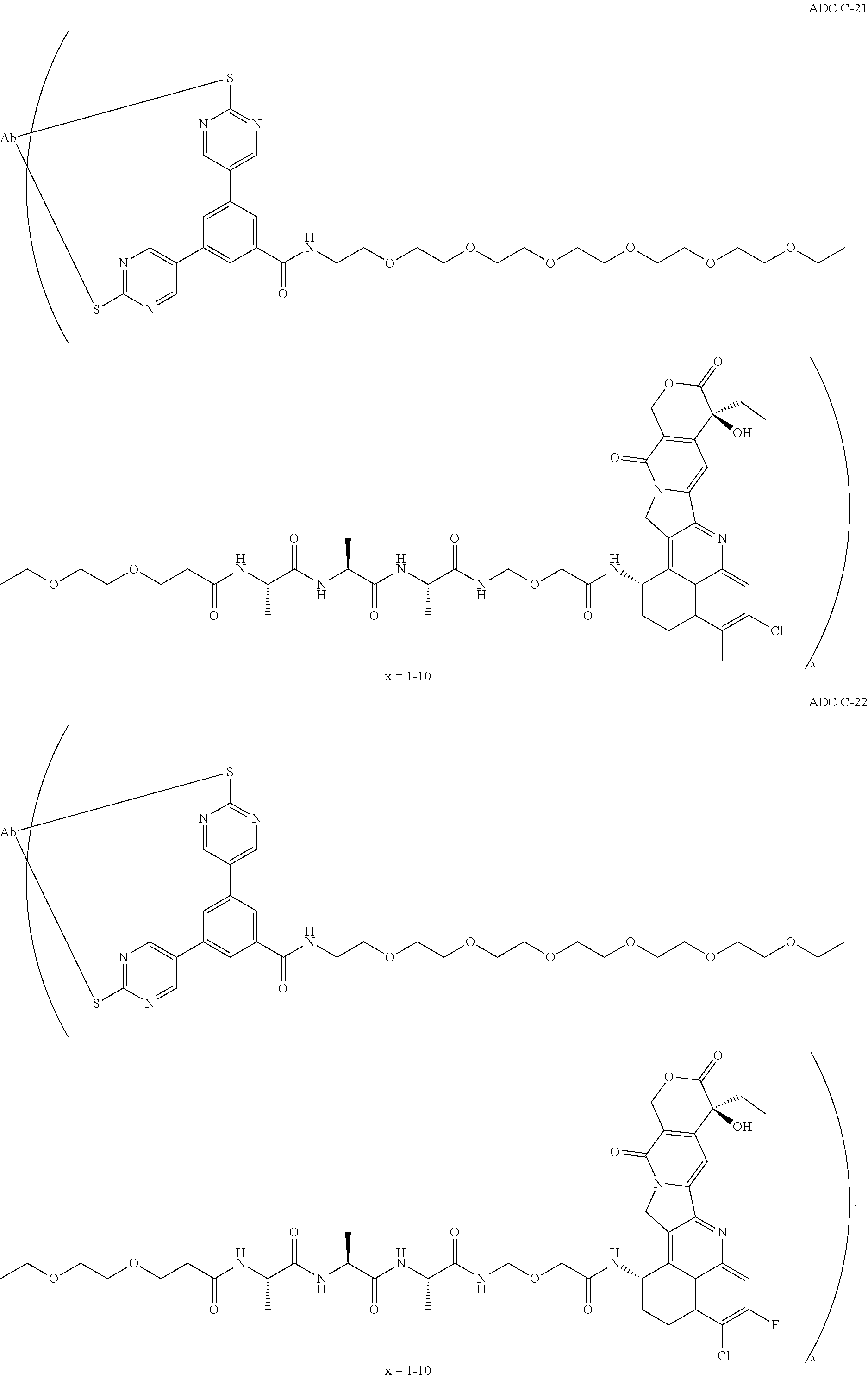

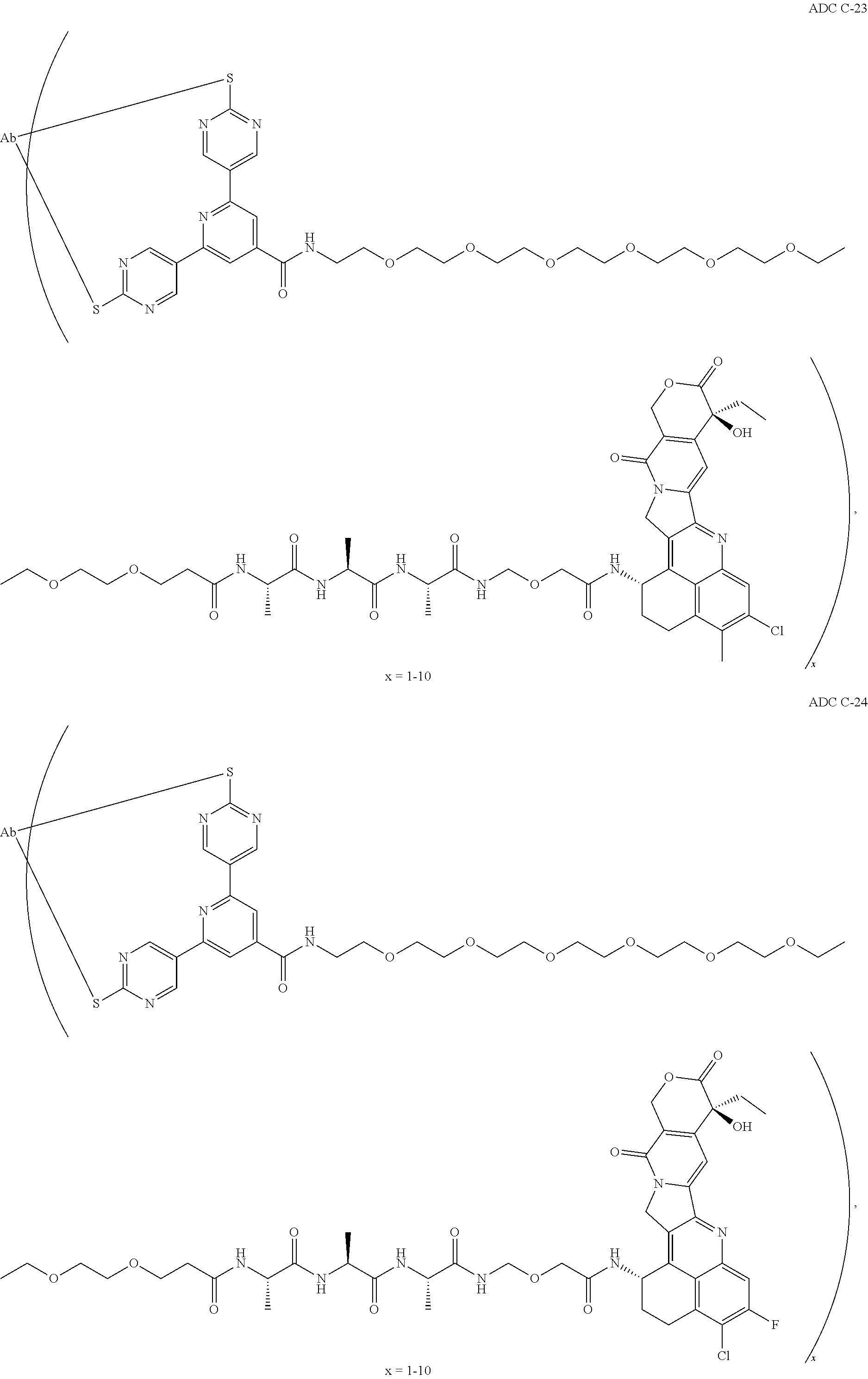

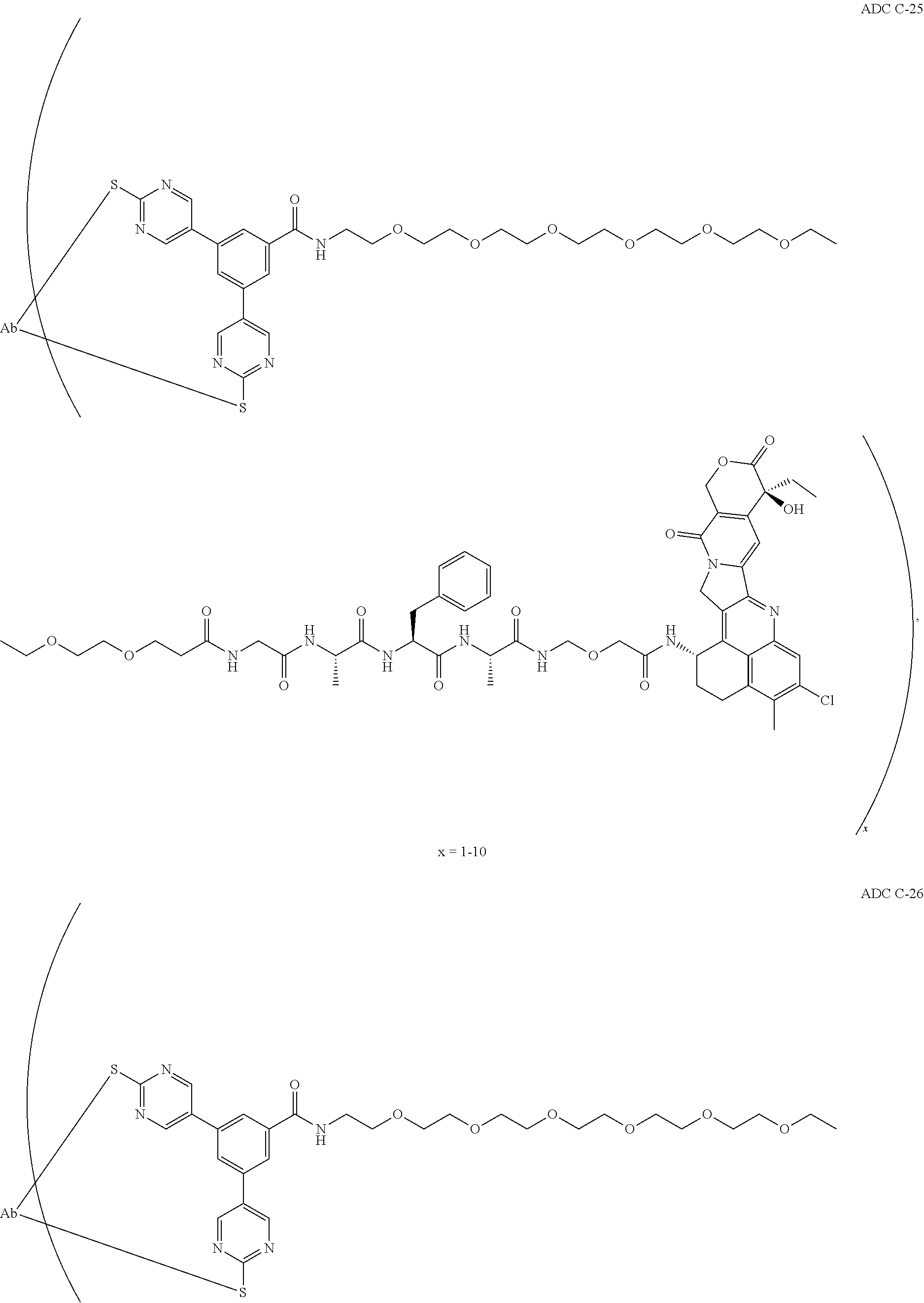

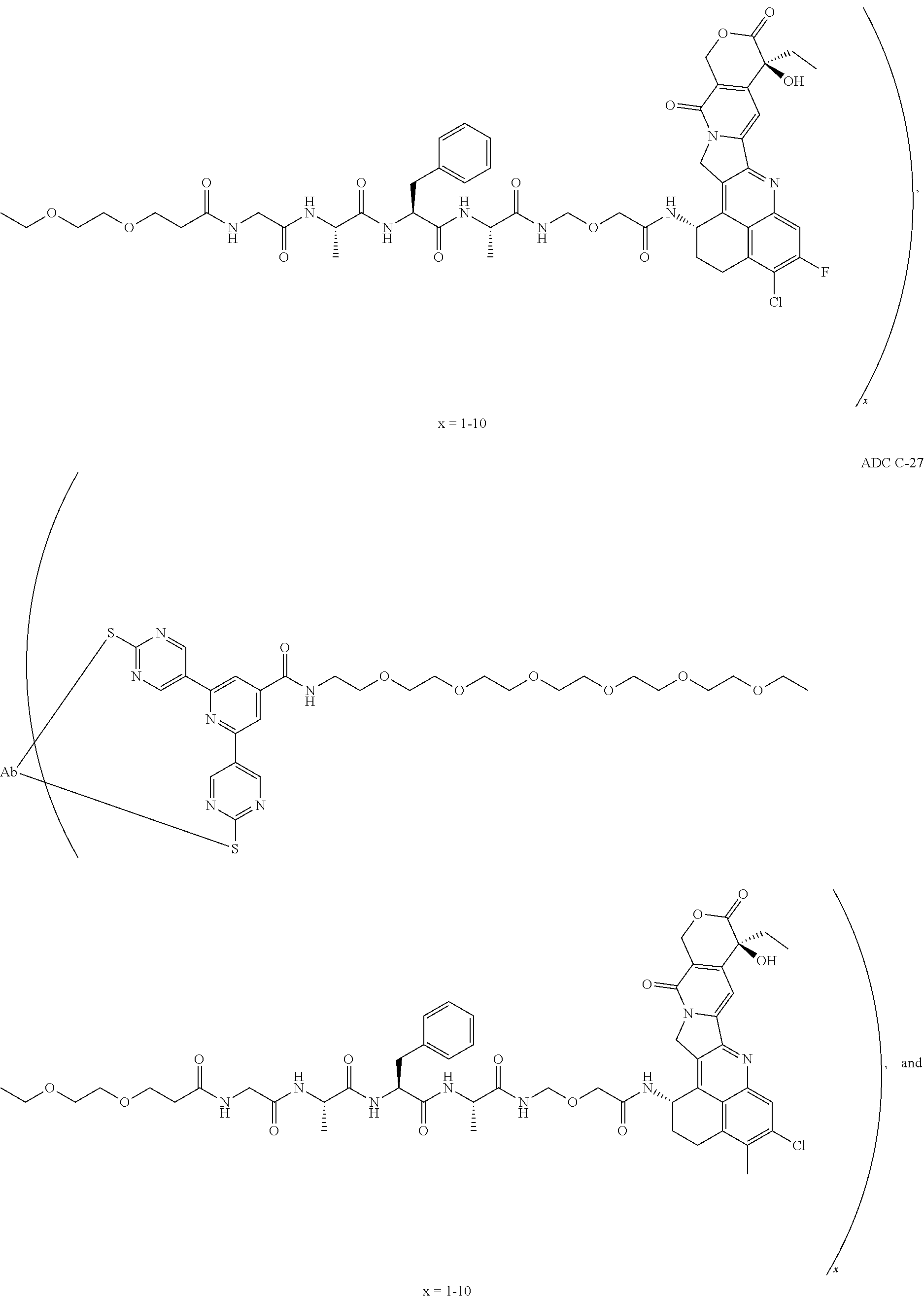

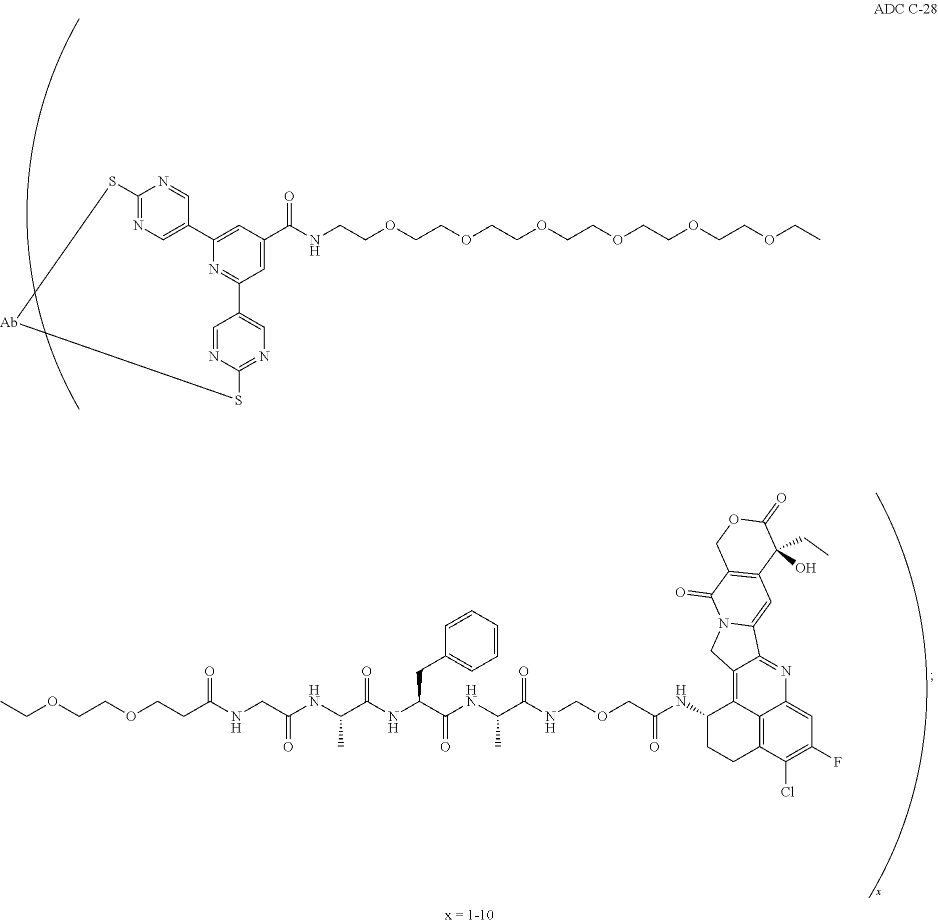

wherein, Ab is a bispecific antibody or an antigen-binding fragment thereof as defined above;

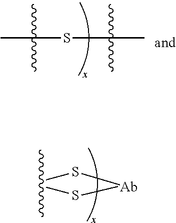

represent the specific connection mode of the sulfhydryl group of a Cys residue in the bispecific antibody or its antigen-binding fragment and M of the antibody-drug conjugate; and x represents the quantity of drug load.

In a further embodiment of the antibody-drug conjugate, the sulfhydryl group in the bispecific antibody or its antigen-binding fragment forms a thioether bond with M of the antibody-drug conjugate through an addition reaction or a substitution reaction to obtain the antibody-drug conjugate.

In a further embodiment of the antibody-drug conjugate, Ab is a bispecific antibody selected from the group consisting of BsAb 07B, BsAb 10B, BsAb 38B, BsAb 41B, BsAb 49B, BsAb 55B, and BsAb 56B.

The present invention further provides an antibody-drug conjugate selected from the group consisting of ADC 07B-A-05, ADC 07B-A-14, ADC 38B-A-14, ADC 49B-A-14, ADC 49B-B-01, ADC 49B-A-05, ADC 41B-A-05, ADC 55B-A-14, and ADC 56B-A-14.

The present invention further provides a composition comprising one or more antibody-drug conjugates disclosed herein. In a further embodiment of the composition, the composition has a DAR value (Drug-Antibody Conjugation Ratio) of 1 to 10. In a further embodiment of the composition, the composition has a DAR value of 1, 2, 3, 4, 5, 6, 7, 8, 9, 10, 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10. In a further embodiment of the composition, the composition has a DAR value of 3 to 9.

In a further embodiment of the composition, the composition has a DAR value of 4 to 8. In a further embodiment of the composition, the composition has a DAR value of 3.0 to 3.5, 3.0 to 4.0, 3.0 to 4.5, 3.0 to 5.0, 3.0 to 5.5, 3.0 to 6.0, 3.5 to 4.0, 3.5 to 4.5, 3.5 to 5.0, 3.5 to 5.5, 3.5 to 6.0, 3.5 to 6.5, 3.5 to 7.0, 3.5 to 7.5, 3.5 to 8.0, 4.0 to 4.5, 4.0 to 5.0, 4.0 to 5.5, 4.0 to 6.0, 4.0 to 6.5, 4.0 to 7.0, 4.0 to 7.5, 4.0 to 8.0, 4.5 to 5.0, 4.5 to 5.5, 4.5 to 6.0, 4.5 to 6.5, 4.5 to 7.0, 4.5 to 7.5, 4.5 to 8.0, 5.0 to 5.5, 5.0 to 6.0, 5.0 to 6.5, 5.0 to 7.0, 5.0 to 7.5, 5.0 to 8.0, 5.5 to 6.0, 5.5 to 6.5, 5.5 to 7.0, 5.5 to 7.5, 5.5 to 8.0, 6.0 to 6.5, 6.0 to 7.0, 6.0 to 7.5, 6.0 to 8.5, 6.5 to 7.0, 6.5 to 7.5, 6.5 to 8.5, 7.0 to 7.5, 7.0 to 9.0, or 7.5 to 9.0.

The present invention further provides a pharmaceutical composition comprising one or more antibody-drug conjugates disclosed herein or a composition disclosed herein, and one or more pharmaceutically acceptable carriers and/or excipients. In a further embodiment of the pharmaceutical composition, the pharmaceutical composition further comprises one or more additional pharmaceutically active agents selected from the group consisting of: EGFR inhibitors, HER2 inhibitors, HER3 inhibitors, HER4 inhibitors, IGFR-1 inhibitors, mTOR inhibitors, PI3 kinase inhibitors, c-MET or VEGF inhibitors, chemotherapy drugs, and any combination thereof.

The present invention further provides a drug combination comprising a first therapeutic agent and a second therapeutic agent, wherein the first therapeutic agent and the second therapeutic agent are administered concurrently or sequentially; the first therapeutic agent is selected from one or more antibody-drug conjugates disclosed herein or a composition disclosed herein; and the second therapeutic agent is selected from the group consisting of an EGFR inhibitor, a HER2 inhibitor, a HER3 inhibitor, a HER4 inhibitor, an IGFR-1 inhibitor, an mTOR inhibitor, a PI3 kinase inhibitor, a c-MET inhibitor, a VEGF inhibitor, a chemotherapy drug, and any combination thereof.

The present invention further provides a use of an antibody-drug conjugate provided herein, a composition provided herein, a pharmaceutical composition provided herein, or a drug combination provided herein in preparation of a medicament, wherein the medicament is used for preventing, treating, and/or acting as an adjuvant in treating diseases related to c-MET and/or EGFR in a subject, and/or to inhibit an activity of c-MET and/or EGFR in vitro or in the body of a subject; wherein the diseases related to c-MET and/or EGFR are associated with EGFR activating mutations, EGFR gene amplification, elevated circulating HGF levels, c-MET activating mutations, and/or c-MET gene amplification, optionally wherein the diseases related to c-MET and/or EGFR comprise cancer.

In a further embodiment of the use, the cancer is selected from epithelial cell carcinoma, breast cancer, ovarian cancer, lung adenocarcinoma, small cell lung cancer, non-small cell lung cancer, oral cancer, colorectal cancer, anal cancer, prostate cancer, bladder cancer, pharyngeal cancer, nasal cancer, pancreatic cancer, skin cancer, tongue cancer, esophageal cancer, vaginal cancer, cervical cancer, spleen cancer, testicular cancer, gastric cancer, thymus cancer, thyroid cancer, hepatocellular carcinoma, sporadic or hereditary papillary renal cell carcinoma, colon cancer, liver cancer, kidney cancer, and head and neck cancer.

In a further embodiment of the use, the antibody-drug conjugate or pharmaceutical composition and an additional pharmaceutically active agent are administered in combination, either simultaneously, separately or sequentially; wherein the additional pharmaceutically active agent is selected from: EGFR inhibitors, HER2 inhibitors, HER3 inhibitors, HER4 inhibitors, IGFR-1 inhibitors, mTOR inhibitors, PI3 kinase inhibitors, c-MET or VEGF inhibitors, chemotherapy drugs, and any combination thereof.

The present invention further provides a method for inhibiting the activity of c-MET and/or EGFR in cells, the method comprising contacting the cells with an antibody-drug conjugate provided herein, a composition provided herein, a pharmaceutical composition provided herein, or a drug combination provided herein; wherein the cells are cells that express c-MET and/or EGFR.

The present invention further provides a method for preventing, treating, and/or acting as an adjuvant in treating a disease associated with c-MET and/or EGFR in a subject in need thereof, the method comprising administering to the subject an effective amount of an antibody-drug conjugate provided herein, a composition provided herein, a pharmaceutical composition provided herein, or a drug combination provided herein, wherein the disease associated with c-MET and/or EGFR is a disease comprising EGFR activating mutations, EGFR gene amplification, elevated circulating HGF levels, c-MET activating mutations, and/or c-MET gene amplification, optionally wherein the disease associated with c-MET and/or EGFR is cancer.

In a further embodiment of the method, the cancer is selected from the group consisting of epithelial cell carcinoma, breast cancer, ovarian cancer, lung adenocarcinoma, small cell lung cancer, non-small cell lung cancer, oral cancer, colorectal cancer, anal cancer, and prostate cancer, bladder cancer, pharyngeal cancer, nasal cancer, pancreatic cancer, skin cancer, tongue cancer, esophageal cancer, vaginal cancer, cervical cancer, spleen cancer, testicular cancer, gastric cancer, thymus cancer, thyroid cancer, hepatocellular carcinoma, sporadic or hereditary papillary renal cell carcinoma, colon cancer, liver cancer, kidney cancer, or head and neck cancer.

In a further embodiment of the method, the method further comprises administering a second therapy to the subject, the second therapy being selected from the group consisting of: surgery, chemotherapy, radiotherapy, immunotherapy, gene therapy, DNA therapy, RNA therapy, nanotherapy, viral therapy, adjuvant therapy and any combination thereof, optionally wherein the second therapy is administered simultaneously, separately, or sequentially.

The present invention further provides an antibody-drug conjugate provided herein, a composition provided herein, a pharmaceutical composition provided herein, or a drug combination provided herein, for preventing, treating, and/or acting as an adjuvant in treating a disease associated with c-MET and/or EGFR in a subject, wherein the disease associated with c-MET and/or EGFR is a disease comprising EGFR activating mutations, EGFR gene amplification, elevated circulating HGF levels, c-MET activating mutations, and/or c-MET gene amplification, optionally wherein the disease associated with c-MET and/or EGFR is cancer. In a further embodiment, the cancer is selected from the group consisting of epithelial cell carcinoma, breast cancer, ovarian cancer, lung adenocarcinoma, small cell lung cancer, non-small cell lung cancer, oral cancer, colorectal cancer, anal cancer, and prostate cancer, bladder cancer, pharyngeal cancer, nasal cancer, pancreatic cancer, skin cancer, tongue cancer, esophageal cancer, vaginal cancer, cervical cancer, spleen cancer, testicular cancer, gastric cancer, thymus cancer, thyroid cancer, hepatocellular carcinoma, sporadic or hereditary papillary renal cell carcinoma, colon cancer, liver cancer, kidney cancer, or head and neck cancer. In a further embodiment, the method further comprises administering a second therapy to the subject, the second therapy being selected from the group consisting of surgery, chemotherapy, radiotherapy, immunotherapy, gene therapy, DNA therapy, RNA therapy, nanotherapy, viral therapy, adjuvant therapy and any combination thereof, optionally wherein the second therapy is administered simultaneously, separately, or sequentially.

### DESCRIPTION OF THE DRAWINGS

FIG. 1A: Schematic diagram of the structure of Fab-scFv-Fc (KIH) bispecific antibody.

FIG. 1B: Schematic diagram of the structure of Fab-scFab-Fc (KIH) bispecific antibody.

FIG. 1C: Schematic diagram of a CrossMab^{VH-VL} bispecific antibody structure.

FIG. 1D: Schematic diagram of the structure of scFv-Fc (KIH) bispecific antibody.

FIG. 2: Efficacy testing of different antibody-drug conjugates on MKN45 models.

FIG. 3: Testing the efficacy of different antibody-drug conjugates on MKN45-EGFR model.

FIG. 4: Testing of the efficacy of different antibody-drug conjugates on MKN45 models.

FIG. 5: Efficacy testing of different antibody-drug conjugates on HCC827 models.

FIG. 6: Testing the efficacy of different antibody-drug conjugates on EBC-1-EGFR model.

FIG. 7: Testing the efficacy of different antibody-drug conjugates on HCC827 model.

FIG. 8: Testing the efficacy of different antibody-drug conjugates on PC-9 model.

FIG. 9: Testing the bystander effect of different antibody-drug conjugates.

### DETAILED DESCRIPTION OF THE INVENTION

### Definition of Terms

In the present invention, unless otherwise stated, scientific and technical terms used herein have the meanings commonly understood by those skilled in the art. Moreover, the cell culture, biochemistry, nucleic acid chemistry, immunology laboratory and other operating procedures used in this disclosure are routine procedures widely used in the corresponding fields. Meanwhile, in order to better understand the present invention, definitions and explanations of relevant terms are provided below.

As used herein, the term “antibody” is used in the broadest sense and includes a variety of antibody structures, including but not limited to monoclonal antibodies, polyclonal antibodies, multispecific antibodies (e.g., bispecific antibodies) and antibody fragments, provided they exhibit the required antigen-binding activity. For example, an immunoglobulin molecule may be composed of two pairs of polypeptide chains, each pair having a light chain (LC) and a heavy chain (HC). Antibody light chains can be classified into kappa (κ) and lambda (λ) light chains. Heavy chains can be classified as mu (μ), delta (δ), gamma (γ), alpha (α), or epsilon (ε), and define the antibody's isotype as IgM, IgD, IgG, IgA, and IgE, respectively. Within the light and heavy chains, the variable and constant regions are connected by a “J” region of approximately 12 or more amino acids, and the heavy chain also contains a “D” region of approximately 3 or more amino acids. Each heavy chain consists of a heavy chain variable region (VH) and a heavy chain constant region (CH). The heavy chain constant region consists of three domains (CH1, CH2 and CH3). Each light chain consists of a VL (VL) and a light chain constant region (CL). The light chain constant region consists of one domain, CL. The constant domains are not directly involved in the binding of antibodies to antigens, but exhibit a variety of effector functions, such as mediating the interaction of immunoglobulins with host tissues or factors, including various cells of the immune system (e.g., effector cells) and the first component of the classical complement system (C1q). The VH and VL regions can also be subdivided into highly variable regions called complementarity-determining regions (CDRs), interspersed with more conservative regions called framework regions (FRs). Each VH and VL is composed of 3 CDRs and 4 FRs arranged from the amino terminus to the carboxyl terminus in the following order: FR1, CDR1, FR2, CDR2, FR3, CDR3, FR4. The variable regions (VH and VL) of each heavy chain/light chain pair respectively form the antigen-binding site. The assignment of amino acids to regions or domains can follow the definition of Kabat, Sequences of Proteins of Immunological Interest (National Institutes of Health, Bethesda, Md. (1987 and 1991)), or Chothia & Lesk (1987) J. Mol. Biol. 196:901-917 and Chothia et al. (1989) Nature 342:878-883.

As used herein, when the term “antibody” is mentioned, it includes not only intact antibodies but also antigen-binding fragments of the antibodies, unless the context clearly indicates otherwise.

The term “antibody” further includes embodiments in which heavy chain constant domains may comprise a C-terminal lysine or lack either a C-terminal lysine or a C-terminal glycine-lysine dipeptide. The term further includes embodiments in which the N-terminal amino acid of the antibody variable domains has undergone cyclization to pyroglutamate. Thus, in a composition comprising antibodies as disclosed herein, various species of the antibodies therein may independently comprise a C-terminal lysine, lack a C-terminal lysine, lack a C-terminal glycine-lysine and/or comprise an N-terminal glutamine or glutamic acid or cyclization of the N-terminal amino acid to pyroglutamate.

As used herein, the term “complementarity determining region” or “CDR” refers to the amino acid residues in the variable region of an antibody that are responsible for antigen binding. The precise boundaries of these amino acid residues can be defined according to various numbering systems known in the art, for example according to the AbM numbering system (Martin A C R, Cheetham J C, Rees A R (1989) Modeling antibody hypervariable loops: A combined algorithm. Proc Natl Acad Sci USA 86:9268-9272), the MacCallum numbering system (MacCallum et al., (1996) J Mol Biol 262: 732-745, see also, e.g., Martin A. “Protein Sequence and Structure Analysis of Antibody Variable Domains,” in Antibody Engineering, Kontermann and Dubel, eds., Chapter 31, pp. 422-439, Springer-Verlag, Berlin (2001)), the AHo numbering system (Honegger and Phückthun, A., J. Mol. Biol. 309:657-670 (2001)), or the IMGT numbering system (Lefranc et al., Dev. Comparat. Immunol. 27:55-77, 2003). For a given antibody, one skilled in the art will readily identify the CDRs defined by each numbering system. Furthermore, the correspondence between different numbering systems is well known to those skilled in the art (see, for example, Lefranc et al., Dev. Comparat. Immunol. 27:55-77, 2003).

In the present invention, the CDRs comprised in an antibody or antigen-binding fragment thereof can be determined according to various numbering systems known in the art, such as Kabat, Chothia, IMGT or AbM numbering systems. In certain embodiments, the CDRs comprised in an antibody or antigen-binding fragment thereof are determined by the Chothia numbering system.

The following general rules disclosed in www.bioinforg.uk: Prof Andrew C. R. Martin's Group and reproduced below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

| Loop | Kabat | AbM | Chothia¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L52 |
| L3 | L89--L97 | L89--L97 | L89--L97 | L89--L96 | L89--L97 |
| H1 | H31--H35B | H26-- | H26-- | H30--H35B | H26-- |
| | (Kabat | H35B | H32 . . . 34 | | H35B |
| | Numbering)³ | | | | |
| H1 | H31--H35 | H26--H35 | H26--H32 | H30--H35 | H26--H33 |
| | (Chothia | | | | |
| | Numbering) | | | | |
| H2 | H50--H65 | H50--H58 | H52--H56 | H47--H58 | H51--H56 |
| H3 | H95--H102 | H95-- | H95--H102 | H93--H101 | H93-- |
| | | H102 | | | H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

The entire amino acid sequence of the VH is commonly numbered according to Kabat, while the three CDRs within the variable region may be defined according to any one of the aforementioned numbering schemes. In particular embodiments, the numbering of the amino acid positions in the VH may be sequential beginning with amino acid position 1 and continuing sequentially to the end of the sequence or according to Kabat. Unless specified otherwise, the amino acid positions in the VH and VL herein are defined according to sequential numbering.

The numbering of the amino acid positions in the heavy chain constant domain may be sequential beginning with amino acid position 1 and continuing sequentially to the end of the sequence or according to Eu numbering. The IgG1 heavy chain constant domain amino acid sequence has 330 amino acids sequentially numbered 1 to 330. The corresponding sequence numbered according to Eu begins with position number 118 and ends with position number 447. Unless specified otherwise, the amino acid positions in the heavy and light chains herein are defined according to sequential numbering.

As used herein, the term “framework region” or “FR” residues refers to those amino acid residues in an antibody variable region other than the CDR residues as defined above.

The term “antibody” is not limited to any particular method of producing the antibody. This includes, for example, recombinant antibodies, monoclonal antibodies, and polyclonal antibodies. The antibodies may be of different isotypes, for example, IgG (e.g., IgG1, IgG2, IgG3 or IgG4 subtypes), IgA1, IgA2, IgD, IgE or IgM antibodies.

As used herein, the term “bispecific antibody” or “BsAb” refers to an antibody with binding specificity for two different antigens (or epitopes), which includes binding specificities for different antigens (or epitopes), e.g., two antigen-binding domains with binding specificities, thereby being able to bind to two different binding sites and/or target molecules. Each antigen-binding domain of a bispecific antibody can be independently selected from a full-length antibody (e.g., an IgG antibody) or an antigen-binding fragment thereof (e.g., Fv, Fab, scFab or scFv). In some cases, the individual antigen-binding domains are linked by a peptide linker.

As used herein, the term “Fv fragment” means an antibody fragment consisting of the VL and VH domains of a single arm of the antibody. Fv fragments are generally considered to be the smallest antibody fragments that can form a complete antigen-binding site. It is generally believed that six CDRs confer the antigen-binding specificity of an antibody. However, even a variable region (such as an Fd fragment, which contains only three CDRs specific for the antigen) can recognize and bind the antigen, although its affinity may be lower than that of the intact binding site.

As used herein, the term “Fc fragment” means a fragment formed by disulfide bonding of the second and third constant regions of the first heavy chain of an antibody to the second and third constant regions of the second heavy chain of antibody fragments. The Fc fragment of an antibody has many different functions but is not involved in antigen binding.

As used herein, the term “scFv” refers to a single polypeptide chain containing VL and VH domains connected by a linker. Such scFv molecules can have a general structure: NH₂—VL-joint-VH—COOH or NH₂—VH-joint-VL-COOH. A suitable prior art peptide linker consists of the repeated GGGGS amino acid sequence (SEQ ID NO: 57) or its variants, e.g., the amino acid sequence (GGGGS)₄ (SEQ ID NO: 58), but variations thereof may also be used. In some cases, a disulfide bond may also exist between the VH and VL of the scFv.

As used herein, the term “Fab fragment” means an antibody fragment consisting of VL, VH, CL, and CH1 domains, which generally consists of one peptide chain containing VL and CL and another peptide chain containing VH and CH1, however, those skilled in the art understand that Fab domains can be arranged according to the native orientation described above, but can also contain domain substitutions or exchanges that promote correct VH and VL pairing (e.g., domain exchanges in the form of CrossMabs). The term “scFab” refers to a single polypeptide chain containing VL, VH, CL, and CH1 domains, in which adjacent domains are optionally connected by a linker. In the typical structure, an scFab contains a single polypeptide chain from N-terminus to C-terminus: (1) VL, CL, VH, and CH1, where CL and VH are usually connected through a peptide linker (e.g., a flexible peptide linker), or (1) VH, CH1, VL, and CL, where CH1 and VL are usually connected through a peptide linker (e.g., a flexible peptide linker).

As used herein, the terms “monoclonal antibody” and “mAb” have the same meaning and are used interchangeably to refer to one from a group of highly homologous antibody molecules or fragments thereof, that is, a group of antibody molecules which are identical except for natural mutations that may occur spontaneously. Monoclonal antibodies are highly specific for a single epitope on an antigen. Polyclonal antibodies, as opposed to monoclonal antibodies, usually refers to antibodies or fragments thereof from a group of antibodies containing at least 2 or more different antibodies that usually recognize different epitopes on an antigen. In addition, the modifier “monoclonal” only indicates that the antibody is characterized as being obtained from a highly homologous population of antibodies and cannot be understood as requiring any specific method to prepare the antibody.

As used herein, the term “CrossMab” refers to a method in the construction of bispecific antibodies that enables the correct association of the light chains and their cognate heavy chains by exchange of heavy-chain and light-chain domains within the antigen-binding fragment (Fab) of one half of the bispecific antibody. This “crossover” retains the antigen-binding affinity but makes the two arms so different that light-chain mispairing can no longer occur. Three possible “CrossMab” formats are: CrossMab^{Fab}, which refers to crossover or exchange in position of the complete VH-CH1 and VL-CL domains of one half of the bispecific antibody; CrossMab^{VH-VL} which refers to the crossover or exchange in position of only the VH and VL domains of one half of the bispecific antibody; and CrossMab^{CH1-CL}, which refers to the crossover or exchange in position of the CH1 and CL domains within the Fab region of one half of the bispecific antibody. CrossMab antibodies have been described or claimed in WO2009080252, WO2009080253, WO2009080251, WO2009080254, WO2010136172, WO2010145792 and WO2013026831. The term “CrossMab” antibody is generally recognized in the art; see, e.g., Brinkmann and Kontennann, MAbs 9(2): 182-212 (2017); Kontermann and Brinkmann, Drug Discovery Today 20(7):838-846 (2015); Schaefer et al., PNAS, 108 11187-1191 (2011); Klein et al., MAbs 8(6):1010-1020 (2016); and Klein et al., MAbs 4(6):653-663 (2012).

As used herein, the term “specific binding” refers to a non-random binding reaction between two molecules, such as the reaction between an antibody and the antigen against which it is directed. The strength or affinity of a specific binding interaction can be expressed as the equilibrium dissociation constant (KD) or half-maximal effect concentration (EC₅₀) of the interaction.

The specific binding properties between two molecules can be determined using methods known in the art. One approach involves measuring the rate at which antigen-binding site/antigen complexes form and dissociate. “Binding rate constant” (kₐ or kₒₙ) and “dissociation rate constant” (k_{dis} or k_{off}) both can be calculated from the concentration and actual rates of association and dissociation (see Malmqvist M, Nature, 1993, 361:186-187). The ratio k_{dis}/kₒₙ is equal to the dissociation constant KD (see Davies et al., Annual Rev Biochem, 1990; 59:439-473). The KD, kₒₙ and k_{dis} values may be measured by any valid method. In certain embodiments, the dissociation constant can be measured using bioluminescence interferometry (e.g., the ForteBio Octet method). Alternatively, surface plasmon resonance techniques (e.g. Biacore) or Kinexa can be used to measure dissociation constants.

As used herein, the term “vector” refers to a nucleic acid delivery vehicle into which a polynucleotide can be inserted. When the vector can express the protein encoded by the inserted polynucleotide, the vector is called an expression vector. The vector can be introduced into the host cell through transformation, transduction or transfection, so that the genetic material elements it carries can be expressed in the host cell. Vectors are well known to those skilled in the art, including but not limited to: Plasmid; phagemid; cosmid; artificial chromosome, such as yeast artificial chromosome (YAC), bacterial artificial chromosome (BAC) or P1-derived artificial chromosome (PAC); phage such as lambda phage or M13 phage and animal viruses, etc. Animal viruses that can be used as vectors include, but are not limited to, retroviruses (including lentiviruses), adenoviruses, adeno-associated viruses, herpesviruses (such as herpes simplex virus), poxviruses, baculoviruses, papillomaviruses, and polyomaviruses (such as SV40). A vector can contain a variety of expression-controlling elements, including, but not limited to, promoter sequences, transcription initiation sequences, enhancer sequences, selection elements, and reporter genes. In addition, the vector may also contain an origin of replication site.

Expression and cloning vectors contain nucleic acid sequences that enable the vector to replicate in one or more selected host cells. Typically, in cloning vectors, this sequence is one that enables the vector to replicate independently of the host chromosomal DNA, and it includes an origin of replication or an autonomously replicating sequence. The term “expression vector” as used herein refers to a vector containing a recombinant polynucleotide that contains expression control sequences operably linked to the nucleotide sequence to be expressed. The expression vector contains sufficient cis-acting elements for expression; other elements for expression can be provided by the host cell or in vitro expression system. Expression vectors include all those known in the art, such as cosmids, plasmids (e.g., naked or contained in liposomes) and viruses (e.g., lentiviruses, retroviruses, adenoviruses, and adeno-associated viruses).

As used herein, the term “host cell” refers to a cell that can be used to introduce a vector, which includes, but is not limited to, prokaryotic cells such as Escherichia coli or Bacillus subtilis, fungal cells such as yeast cells or Aspergillus, insect cells such as S2, Drosophila cells, or Sf9, or animal cells such as fibroblasts, NS0 cells, Vero cells, Hela cells, COS cells, CHO cells (such as CHO-K1, CHO-S, CHO DXB11, ExpiCHO, or CHO DG44 cells), ExpiCHO cells, HEK293 cells, Expi293 cells, BHK cells, and MDCKII cells, etc.

As used herein, the term “identity” is used to refer to a match of sequences between two polypeptides or between two nucleic acids. When a position in both sequences being compared is occupied by the same base or amino acid monomer subunit (for example, a position in each of two DNA molecules is occupied by adenine, or a position in each of two polypeptides is occupied by lysine), then the molecules are identical at that position. “Percent identity” between two sequences is a function of the number of matching positions common to the two sequences divided by the number of positions compared×100. For example, if 6 out of 10 positions of two sequences match, then the two sequences are 60% identical. For example, the DNA sequences CTGACT and CAGGTT share 50% identity (3 positions match out of 6 total positions). Typically, comparisons are made when two sequences are aligned to yield maximum identity. Such alignments can be achieved using, for example, the method of Needleman et al. (1970) J. Mol. Biol. 48: 443-453 which can be conveniently performed by computer programs such as the Align program (DNAstar, Inc.). The algorithm of E. Meyers and W. Miller (Comput. The algorithm of Appl Biosci., 4:11-17 (1988)) which has been incorporated into the ALIGN program (version 2.0) can also be used to determine the percent identity between two amino acid sequences by using the PAM120 weight residue table, a gap length penalty of 12, and a gap penalty of 4. Alternatively, the Needleman algorithm has been integrated into the GAP program of the GCG software package (available at www.gcg.com) and can be used to determine the percent identity between two amino acid sequences by using the Blossum 62 matrix or PAM250 matrix with a gap weight of 16, 14, 12, 10, 8, 6 or 4 and a length weight of 1, 2, 3, 4, 5 or 6.

The twenty conventional amino acids involved in this disclosure have been prepared following conventional usage. See, e.g., Immunology-A Synthesis (2nd Edition, E. S. Golub and D. R. Gren, Eds., Sinauer Associates, Sunderland, Mass. (1991)), which is incorporated herein by reference. In the present disclosure, the terms “polypeptide” and “protein” have the same meaning and are used interchangeably. And in the present disclosure, amino acids are generally represented by one-letter and three-letter abbreviations well known in the art. For example, alanine can be represented by A or Ala.

As used herein, the term “pharmaceutically acceptable carrier and/or excipient” refers to a carrier and/or excipient that is pharmacologically and/or physiologically compatible with the subject and the active ingredient. It is well known in the art (see, e.g., Remington's Pharmaceutical Sciences. Edited by Gennaro A R, 19th ed. Pennsylvania: Mack Publishing Company, 1995), and includes but is not limited to: pH adjusters, surfactants, adjuvants, ionic strength enhancers, diluents, agents to maintain osmotic pressure, agents to delay absorption, preservatives. For example, pH adjusting agents include, but are not limited to, phosphate buffer. Surfactants include, but are not limited to, cationic, anionic or nonionic surfactants such as Tween-80. Ionic strength enhancers include, but are not limited to, sodium chloride. Preservatives include, but are not limited to, various antibacterial and antifungal agents, such as parabens, chlorobutanol, phenol, sorbic acid, etc. Agents that maintain osmotic pressure include, but are not limited to, sugar, NaCl, and the like. Agents that delay absorption include, but are not limited to, monostearate and gelatin. Diluents include, but are not limited to, water, aqueous buffers (such as buffered saline), alcohols and polyols (such as glycerol), and the like. Stabilizer has the meaning generally understood by those skilled in the art, and a stabilizer can stabilize the desired activity of active ingredients in medicines, including but not limited to sodium glutamate, gelatin, SPGA, sugars (such as sorbitol, mannitol, starch, sucrose, lactose, dextran, or glucose), amino acids (such as glutamic acid, glycine), proteins (such as dry whey, albumin or casein) or their degradation products (such as lactalbumin hydrolyzate), etc.

As used herein, the terms “DAR” or “Drug Antibody Ratio” or “Drug-Antibody Conjugate Ratio” as used herein interchangeably, refer to the average number of linker/payload moieties attached to the antibodies present in a composition. For a composition comprising an ADC of the present disclosure, the DAR for the composition is the average of the DARs (linker-payload moieties of all of the individual ADC molecules present in said composition), and this average is expressed as an integer or a decimal. As such, in some embodiments for a composition comprising an ADC of the present disclosure, the DAR of the composition is an integer or a decimal from 1, 2, 3, 4, 5, 6, 7, 8, 9, 10, 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10. In additional embodiments, for a composition comprising an ADC of the present disclosure, the DAR of the composition is an integer or a decimal from 3.0 to 3.5, 3.0 to 4.0, 3.0 to 4.5, 3.0 to 5.0, 3.0 to 5.5, 3.0 to 6.0, 3.5 to 4.0, 3.5 to 4.5, 3.5 to 5.0, 3.5 to 5.5, 3.5 to 6.0, 3.5 to 6.5, 3.5 to 7.0, 3.5 to 7.5, 3.5 to 8.0, 4.0 to 4.5, 4.0 to 5.0, 4.0 to 5.5, 4.0 to 6.0, 4.0 to 6.5, 4.0 to 7.0, 4.0 to 7.5, 4.0 to 8.0, 4.5 to 5.0, 4.5 to 5.5, 4.5 to 6.0, 4.5 to 6.5, 4.5 to 7.0, 4.5 to 7.5, 4.5 to 8.0, 5.0 to 5.5, 5.0 to 6.0, 5.0 to 6.5, 5.0 to 7.0, 5.0 to 7.5, 5.0 to 8.0, 5.5 to 6.0, 5.5 to 6.5, 5.5 to 7.0, 5.5 to 7.5, 5.5 to 8.0, 6.0 to 6.5, 6.0 to 7.0, 6.0 to 7.5, 6.0 to 8.5, 6.5 to 7.0, 6.5 to 7.5, 6.5 to 8.5, 7.0 to 7.5, 7.0 to 9.0, or 7.5 to 9.0. The term “composition” as used above, is understood to encompass pharmaceutical compositions. Average DAR can be determined by various conventional means such as UV spectroscopy, mass spectroscopy, ELISA assay, radiometric methods, hydrophobic interaction chromatography (HIC), electrophoresis, and HPLC.

As used herein, the term “prevention” refers to a method performed to prevent or delay the occurrence of a disease or condition or symptom (e.g., tumor) in a subject. As used herein, the term “treatment” refers to a method performed to obtain a beneficial or desired clinical result. For the purposes of this disclosure, beneficial or desired clinical results include, but are not limited to, alleviation of symptoms, reduction of the extent of the disease, stabilization (i.e., no worsening) of the disease state, delaying or slowing the progression of the disease, ameliorating or alleviating the symptoms of the disease status, and relief of symptoms (whether partial or complete), whether detectable or undetectable. In addition, “treatment” may also refer to prolonging survival compared with expected survival if no treatment was received.

As used herein, the term “subject” refers to a mammal, such as a primate mammal, such as a human. In certain embodiments, the subject (e.g., human) has a tumor, or is at risk of suffering from a disease described herein.

As used herein, the term “effective amount” refers to an amount sufficient to achieve, or at least partially achieve, the desired effect. For example, a prophylactically effective amount refers to an amount sufficient to prevent or delay the occurrence of a disease (e.g., tumor); a therapeutically effective amount refers to an amount sufficient to cure or at least partially prevent an existing disease or its complications. Determining such effective amounts is well within the capabilities of those skilled in the art. For example, the amount effective for therapeutic use will depend on the severity of the disease to be treated, the overall status of the patient's own immune system, the patient's general condition such as age, weight and gender, the manner in which the drug is administered, other treatments administered concurrently, etc.

As used herein, the term “effector function” refers to those biological activities that are attributable to the Fc domain of an antibody (either a native sequence Fc domain or an amino acid sequence variant Fc domain) and which vary with the antibody's Fc domain. Examples of antibody effector functions include, but are not limited to: Fc receptor binding affinity, antibody-dependent cell-mediated cytotoxicity (ADCC), complement-dependent cytotoxicity (CDC), antibody-dependent cellular phagocytosis (ADCP), cell surface receptor (e.g., B cell receptor) binding, B cell activation, cytokine secretion, half-life/clearance rate of antibodies and antigen-antibody complexes, etc. Methods of altering the effector function of antibodies are known in the art, for example by introducing mutations in the Fc domain.

As used herein, the term “antibody-dependent cell-mediated cytotoxicity (ADCC)” refers to a form of cytotoxicity in which Ig interacts with the Fc receptor (FcR) present on cytotoxic cells (e.g., natural killer (NK) cells, neutrophils, macrophages), allowing these cytotoxic effector cells to specifically bind to antigen-attached target cells and then kill the target cells by secreting cytotoxins.

As used herein, combination therapy includes combining a bispecific antibody or pharmaceutical composition of the present disclosure with one or more additional active therapeutic agents of a second therapy (e.g., a chemotherapeutic agent) or other prophylactic or therapeutic modalities (e.g., radiotherapy).

In such combination therapies, the various active agents often have different complementary mechanisms of action, and the combination therapy may result in synergistic effects. Combination therapies include therapeutic agents that affect the immune response (e.g., enhance or activate the response) and therapeutic agents that affect (e.g., inhibit or kill) tumors/cancer cells. Combination therapy reduces the likelihood of drug-resistant cancer cells developing. Combination therapy may allow for dose reduction of one or more of the agents to reduce or eliminate adverse effects associated with one or more of the agents. Such combination therapies may have a synergistic therapeutic or preventive effect on the underlying disease, disorder or condition.

As used herein, “combination” includes therapies that may be administered separately, for example, formulated separately for separate administration (e.g., may be provided in a kit), as well as therapies that may be administered together in a single formulation (i.e., a “co-formulation”). In certain embodiments, bispecific antibodies of the present disclosure can be administered sequentially. In other embodiments, bispecific antibodies can be administered simultaneously. The bispecific antibodies of the present disclosure may be used in any combination with at least one other (active) agent.

The terms “cancer” and “tumor” are used interchangeably and refer to a large group of diseases characterized by the uncontrolled growth of abnormal cells in the body. Unregulated cell division may lead to the formation of malignant tumors or cells that invade adjacent tissues and may metastasize to distant parts of the body via the lymphatic system or bloodstream. Cancer includes benign and malignant cancers as well as dormant tumors or micrometastases. Cancer also includes hematological malignancies.

The term “alkyl” refers to a group obtained by removing one hydrogen atom from a straight-chain or branched hydrocarbon group, such as “C₁₋₂₀ alkyl”, “C₁₋₁₀ alkyl”, “C₁₋₆ alkyl”, “C₁₋₄ alkyl”, “C₁₋₃ alkyl”, etc. Specific examples include but are not limited to: Methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 3-methylpentyl, 2-methylpentyl, 1-methylpentyl, 3,3-dimethylbutyl, 2,2-dimethyl butyl, 1,1-dimethylbutyl, 1,2-dimethylbutyl, 1,3-dimethylbutyl, 2,3-dimethylbutyl, 2-ethylbutyl, 1,2-dimethylpropyl, etc.

The term “alkylene” refers to a group obtained by removing 2 hydrogen atoms from a straight-chain or branched hydrocarbon group, such as “C₁₋₂₀ alkylene”, “C₁₋₁₀ alkylene”, “C₃₋₁₀ alkylene”, “C₅₋₈ alkylene”, “C₁₋₆ alkylene”, “C₁₋₄ alkylene”, “C₁₋₃ alkylene”, etc. Specific examples include but are not limited to: methylene, ethylene, 1,3-propylene, 1,4-butylene, 1,5-pentylene or 1,6-hexylene, etc.

The term “alkenylene” refers to a divalent group obtained by losing two hydrogen atoms from a linear or branched hydrocarbon group containing at least one carbon-carbon double bond, including, for example, “C₂₋₂₀ alkenylene”, “C₃₋₁₀ alkenylene”, “C₅₋₈ alkenylene” etc. Examples include but are not limited to: ethenylene, 1-propenylene, 2-propenylene, 1-butenylene, 2-butenylene, 1,3-butadienylene, 1-pentenylene, 2-pentenylene, 3-pentenylene, 1,3-pentadienylene, 1,4-pentadienylene, 1-hexenylene, 2-hexenylene, 3-hexenylene, 1,4-hexadienylene, etc.

The term “alkynylene” refers to a divalent group obtained by losing two hydrogen atoms from a straight or branched chain hydrocarbon group containing at least one carbon-carbon triple bond. Includes e.g. “C₂₋₂₀ alkynylene”, “C₃₋₁₀ “alkynylene”, “C₅₋₈ alkynylene” etc. Examples include but are not limited to: ethynylene, 1-propynylene, 2-propynylene, 1-butynylene, 2-butynylene, 1,3-butadiynylene, 1-pentynylene, 2-pentynylene, 3-pentynylene, 1,3-pentadiynylene, 1,4-pentadyinylene, 1-hexynylene, 2-hexynylene, 3-hexynylene, 1,4-hexadiynylene, etc.

The term “aliphatic heterocycle” refers to a saturated or partially saturated cyclic structure containing at least one ring member selected from N, O, and S. Specific examples include but are not limited to 5-6 membered aliphatic heterocycles, 5-6 membered nitrogen-containing aliphatic heterocycles, 5-6 membered oxygen-containing aliphatic heterocycles, etc., such as tetrahydrofuran, pyrrolidine, piperidine, tetrahydropyran, etc.

The term “heteroaromatic ring” refers to an aromatic cyclic structure containing at least one ring member selected from N, O, and S. Specific examples include but are not limited to 5-6 membered aromatic heterocycles, 5-6 membered nitrogen-containing aromatic heterocycles, 5-6 membered oxygen-containing aromatic heterocycles, etc., such as furan, thiophene, pyrrole, thiazole, isothiazole, thiadiazole, oxaazole, isooxaazole, oxadiazole, imidazole, pyrazole, 1,2,3-triazole, 1,2,4-triazole, 1,2,3-oxadiazole, 1,2,4-oxadiazole, 1,2, 5-oxadiazole, 1,3,4-oxadiazole, pyridine, pyrimidine, pyridazine, pyrazine, 1,2,3-triazine, 1,3,5-triazine, 1,2,4,5-tetrazine, etc.

The term “aromatic ring system” refers to a monocyclic or polycyclic system containing at least one aromatic ring (such as benzene ring, etc.) or heteroaromatic ring (such as pyrimidine ring, etc.), wherein two or more aromatic rings and/or heteroaromatic rings can form a fused ring or be connected by a single bond (such as dipyrimidinylphenyl, etc.). The aromatic ring system can be divalent or higher (such as trivalent or tetravalent), such as 5-20 membered aromatic ring system.

### Abbreviations

The abbreviations used in herein have the following meanings:

| Abbreviation | Meaning | | |
|---|---|---|---|
| CDR | Complementarity determining regions in immunoglobulin variable regions | | |
| FR | Antibody framework region: Amino acid residues other than CDR residues in | | |
| | the antibody variable region | | |
| VH | Antibody heavy chain variable region | | |
| VL | Antibody light chain variable region | | |
| IgG | Immunoglobulin G | | |
| IMGT | The numbering system based on The international ImMunoGeneTics | | |
| | information system ® (IMGT) initiated by Lefranc et al. can be found in | | |
| | Lefranc et al., Dev. Comparat. Immunol. 27: 55-77, 2003. | | |
| Kabat | Immunoglobulin alignment and numbering system proposed by Elvin A. | | |
| | Kabat (see, e.g., Kabat et al., Sequences of Proteins of Immunological | | |
| | Interest, 5th Ed. Public Health Service, National Institutes of Health, | | |
| | Bethesda, Md., 1991). | | |
| Chothia | The immunoglobulin numbering system proposed by Chothia et al. is a classic | | |
| | rule for identifying CDR region boundaries based on the position of structural | | |
| | loop regions (see, e.g., Chothia & Lesk (1987) J. Mol. Biol. 196: 901-917; | | |
| | Chothia et al. (1989) Nature 342: 878-883). | | |
| AbM | The definition of CDR comes from Martin's related research (Martin ACR, | | |
| | Cheetham JC, Rees AR (1989) Modeling antibody hypervariable loops: A | | |
| | combined algorithm. Proc Natl Acad Sci USA 86: 9268-9272). | | |
| mAb | Monoclonal antibodies | | |
| EC₅₀ | Concentration that produces 50% efficacy or binding | | |
| IC₅₀ | Concentration that produces 50% inhibition | | |
| ELISA | Enzyme-linked immunosorbent assay | | |
| PCR | Polymerase chain reaction | | |
| HRP | Horseradish peroxidase | | |
| KD | Equilibrium dissociation constant | | |
| Ka | Binding rate constant | | |
| Kd | Dissociation rate constant | | |
| ADCC | Antibody-dependent cell-mediated cytotoxicity | | |
| CDC | Complement-dependent cytotoxicity | | |
| FACS | Flow cytometry technology | | |
| CDR-H1 | Complementarity determining region 1 in the immunoglobulin heavy chain | | |
| | variable region | | |
| CDR-H2 | Complementarity determining region 2 in the immunoglobulin heavy chain | | |
| | variable region | | |
| CDR-H3 | Complementarity determining region 3 in the immunoglobulin heavy chain | | |
| | variable region | | |
| CDR-L1 | Complementarity determining region 1 in the immunoglobulin light chain | | |
| | variable region | | |
| CDR-L2 | Complementarity determining region 2 in the immunoglobulin light chain | | |
| | variable region | | |
| CDR-L3 | Complementarity determining region 3 in the immunoglobulin light chain | | |
| | variable region | | |
| Abbreviation | Meaning | Abbreviation | Meaning |
| HATU | N,N,N′,N′-tetramethyl-O-(7- | HOBt | 1-Hydroxybenzotriazole |
| | azabenzotriazol-1-yl)urea | | |
| | hexafluorophosphate | | |
| DIPEA | Diisopropylethylamine | EDCl | 1-(3-Dimethylaminopropyl)- |
| | | | 3-ethylcarbodiimide |
| | | | hydrochloride |
| TFA | Trifluoroacetic acid | DMF | N,N-dimethylformamide |
| Pd/C | Palladium carbon | DMTMM | 4-(4,6-Dimethoxytriazin-2- |
| | | | yl)-4-methylmorpholine |
| | | | hydrochloride |
| NBS | N-bromosuccinimide | IPA | Isopropyl alcohol |
| NaIO₄ | Sodium periodate | RuCl₃•H₂O | Rutheniumtrichloride hydrate |
| XPhos Pd G3 | (2-dicyclohexylphosphino-2′, | LiOH•H₂O | Lithium hydroxide hydrate |
| | 4′,6′-tri-isopropyl-1,1′- | | |
| | biphenyl) (2′-amino-1,1′- | | |
| | biphenyl-2-yl)palladium(II) | | |
| | methanesulfonate | | |
| K₃PO₄ | Potassium phosphate | m-CPBA | Metachloroperoxybenzoic |
| | | | acid |
| THF | Tetrahydrofuran | DMSO | Dimethyl sulfoxide |
| DCM | Dichloromethane | MeOH | Methanol |
| HPLC | HPLC | LC-MS | LC/MS |
| TCEP | (tris(2-carboxyethyl) | Na₂HPO₄ | Disodium phosphate |
| | phosphine | | |
| EDTA | Ethylenediaminetetraacetic | PB | Phosphate buffer |
| | acid | | |
| DAR | Drug loading ratio | | |

### Antibody-Drug Conjugates (ADCs)

In one aspect, the present disclosure provides an ADC having a structure represented by the formula Ab-[M-L-E-D]ₓ, wherein:
Ab is a bispecific antibody or bispecific antigen-binding fragment thereof comprising a first antigen-binding domain specifically binding c-MET and a second antigen-binding domain specifically binding EGFR;
M is a linker site connected to the bispecific antibody or bispecific antigen-binding fragment thereof,
L is a structural fragment connecting the linker sites M and E;
E is a structural fragment connecting L and D;
D is a cytotoxic drug fragment; and
x is selected as any integer from 1 to 10.

### Antibodies of the ADCs

In certain embodiments, the bispecific antibody of the ADC is a bispecific antibody that has high affinity for both EGFR and c-MET, can specifically recognize/bind EGFR and c-MET, and may block the binding of EGF to EGFR and/or the binding of HGF to c-MET, thus blocking the downstream signaling pathways of EGFR and/or c-MET at the same time, causing inhibition of cell proliferation.

In some embodiments, the first antigen-binding domain of the bispecific antibody comprises a first light chain variable region (VL) and a first heavy chain variable region (VH), and the first VL and the first VH collectively form a domain capable of specifically binding to c-MET; and the second antigen-binding domain comprises a second VL and a second VH, and the second VL and the second VH together form a domain capable of specifically binding to EGFR.

In certain embodiments, the first antigen-binding domain and the second antigen-binding domain of the bispecific antibody are each independently selected from an scFv, a Fab, and an scFab.

Unless otherwise explicitly stated, Fab domains may be arranged according to the their native orientation or may comprise domain substitutions or exchanges that promote correct VH and VL pairing (e.g., domain exchanges in the form of CrossMabs).

CrossMab is a method for preventing mispairing of light and heavy chains, see US patent U.S. Pat. No. 9,266,967B2, which is incorporated herein by reference in its entirety. This method mainly utilizes the principle that the same parts of an antibody repel each other, that is, VH and VH repel each other, CH1 and CH1 repel each other, CL and CL repel each other, and VL and VL repel each other. The purpose of preventing light chain mismatch is achieved by regional interchange of the heavy and light chains of one of the Fab in bispecific antibodies (e.g., light chain CL-VL is exchanged with heavy chain CH1-VH, VL and VH are interchanged, or CH and CL are interchanged).

Therefore, in certain embodiments, the domain exchange in the CrossMab format is selected from:
CrossMab^{Fab}: wherein, in the Fab of one of the two heavy chains of the antibody, the antibody light chain CL-VL thereof is interchanged with the antibody heavy chain CH1-VH of the Fab;
CrossMab^{VH-VL}: wherein, in the Fab of one of the two heavy chains of the antibody, the VL and VH of the antibody Fab are interchanged; and
CrossMab^{CH1-CL}: wherein, in the Fab of one of the two heavy chains of the antibody, the CH and CL of the antibody Fab are interchanged.

In certain embodiments, the bispecific antibody further comprises an Fc domain (Fc). In certain embodiments, the Fc domain comprises a first Fc domain monomer and a second Fc domain monomer. In certain embodiments, the first and second Fc domain monomers each independently comprise one or more amino acid modifications capable of promoting heterodimerization of the first and second Fc domain monomers over homodimerization.

In certain embodiments, the Fc domain comprises a first Fc domain monomer comprising amino acid modifications capable of forming a knob structure and a second Fc domain monomer comprising amino acid capable of forming a hole structure corresponding to the position of the knob structure in the first Fc domain monomer, wherein the hole structure can pair with the knob structure to form a heterodimeric Fc domain (Fc heterodimer).

Those skilled in the art can easily understand that the hole structure can be paired with the knob structure to form a “knob-in-hole (KIH) structure”. The KIH structure is formed by introducing amino acid modifications that can form a “knob” structure and a “hole” structure at corresponding positions in the CH3 domains of two Fc domain monomers through amino acid substitutions so as to form two Fc domain monomers that can only form heterodimeric Fc domains having the knob-in-hole structure. Methods for reducing heavy chain mismatching in bispecific antibodies by targeting specific interaction interfaces between monomers (see Ridgway et al., Protein Eng., 9:617-621(1996); WO2006028936; the entire text of which is incorporated herein by reference), due to the mutual attraction between the “knob” structure and the “hole” structure, and the mutual repulsion between the “knob” structure and the “knob” structure, thus can effectively reduce heavy chain mismatching in bispecific antibodies.

Amino acid modifications that promote formation of heterodimers have been disclosed in WO9850431, which discloses a KIH method for creating Fc domain monomer heterodimers. In the KIH method, one Fc domain monomer of the heterodimer pair comprises amino acid substitutions that create a protuberance that extends outward from surface of the Fc domain monomer (knob) that fits into a hole created by appropriate amino acid substitutions in the other Fc domain monomer of the heterodimeric pair, which promotes heterodimer formation over homodimer formation. An example of amino acid substitutions include S354C:T366W amino acid substitutions in a first Fc domain monomer to form the knob, and include Y349C:T366S:L368A:Y407V amino acid substitutions in a second Fc domain monomer to form the hole (amino acid numbering according to EU Index) wherein the first and second Fc domain monomers form a heterodimer pair. WO2014084607 discloses KIH in which the Fc heterodimer comprises a first Fc domain monomer comprising a K409W amino acid substitution to form the knob and a second Fc domain monomer comprising D399V and F405T amino acid substitutions to form the hole (amino acid numbering according to EU Index) wherein the first and second Fc domain monomers form a heterodimer pair. WO2013063702 discloses KIH in which the Fc heterodimer comprises a first Fc domain monomer comprising amino acid modifications at positions T350, L351, F405, and Y407, and the second Fc domain monomer comprises amino acid modifications at positions T350, T366, K392 and T394 (amino acid numbering according to EU Index) wherein the first and second Fc domain monomers form a heterodimer pair.

In certain embodiments, the Fc domain monomer is derived from the Fc of human immunoglobulin and is modified to provide a pair of Fc domain monomers capable of forming a heterodimer pair. For example the amino acid sequence of a first Fc domain monomer is modified in a specific region thereof to provide a knob structure and the amino acid sequence of a second Fc domain monomer is modified in the corresponding region to provide a hole structure capable of accepting the knob structure on the first Fc domain monomer, thereby enabling the first and second Fc domain monomers to form a heterodimer pair while preventing the first and second Fc domain monomers from forming homodimer pairs. In certain embodiments, the human immunoglobulin is IgG1, IgG2, IgG3, or IgG4. In certain embodiments, the Fc domain monomer comprises one or more modifications that form a knob structure, wherein the one or more modifications comprise S354C and/or T366W amino acid substitutions, wherein the numbering is according to the Eu numbering scheme. In certain embodiments, the Fc domain monomer comprises S354 and T366W amino acid substitutions, wherein the numbering is according to the Eu numbering scheme. In certain embodiments, the Fc domain monomer comprises one or more modifications that form a hole structure, wherein the one or more modifications comprise Y349C, T366S, L368A, and/or Y407V amino acid substitutions, wherein the numbering is according to the Eu numbering scheme. In certain embodiments, the Fc domain monomer comprises Y349C, T366S, L368A, and Y407V amino acid substitutions, wherein the numbering is according to the Eu numbering scheme.

In certain embodiments, the Fc domain monomers when in the form of an Fc heterodimer further display altered effector function (e.g., enhanced antibody-dependent cell-mediated cytotoxicity (ADCC) activity) compared to a wild-type Fc domain. The altered effector function may be introduced by, for example, mutation or chemical modification of the Fc domain monomers comprised in the Fc heterodimer.

In further embodiments, the first and second Fc domain monomers further comprise one or more amino acid substitutions that reduce or ablate effector function of the Fc domain. In particular embodiments, the first and second Fc domain monomers further comprise: (i) E233A and L235A (EALA) amino acid substitutions, wherein the numbering is according to the Eu numbering scheme; (ii) L234A and L235A (LALA) amino acid substitutions, wherein the numbering is according to the Eu numbering scheme; (iii) L234A L235A D265S (LALADS) amino acid substitutions, wherein the numbering is according to the Eu numbering scheme; (iv) L234A L235A P329G (LALAPG) amino acid substitutions, wherein the numbering is according to the Eu numbering scheme; (v) L235E (LE) amino acid substitutions, wherein the numbering is according to the Eu numbering scheme; (vi) D265A (DS) amino acid substitution, wherein the numbering is according to the Eu numbering scheme; (vii) D265A N297G (DANG) amino acid substitutions, wherein the numbering is according to the Eu numbering scheme; (viii) N297X amino acid substitution, wherein X is any amino acid other than N, wherein the numbering is according to the Eu numbering scheme; (ix) N297A/D356E/L358M (NADELM) amino acid substitutions, wherein the numbering is according to Eu numbering scheme; or (x) D356E L358M (DELM) amino acid substitutions, wherein the numbering is according to Eu numbering scheme.

In certain embodiments, the first Fc domain monomer comprises the amino acid sequence shown in SEQ ID NO: 49 or 51, and the second Fc domain monomer comprises the amino acid sequence shown in SEQ ID NO: 50 or 52.

In certain embodiments, a first antigen-binding domain and a second antigen-binding domain are each linked to one of the first and second Fc domain monomers of the Fc heterodimer.

In certain embodiments, the first antigen-binding domain is linked to the first Fc domain monomer and the second antigen-binding domain is linked to the second Fc domain monomer; or the first antigen-binding domain is linked to the second Fc domain monomer, and the second antigen-binding domain is linked to the first Fc domain monomer.

The individual CDRs of an antigen-binding domain of a bispecific antibody of an ADC provided herein can be determined according to any CDR numbering scheme known in the art.

In certain embodiments, an antigen-binding domain of a bispecific antibody of an ADC provided herein comprises the light chain CDR 1 (CDR-L1), CDR-L2, and/or CDR-L3 of a VL amino acid sequence set forth in any one of SEQ ID NOs: 15, 17, and 59 as determined by the Kabat numbering scheme. In certain embodiments, an antigen-binding domain of a bispecific antibody of an ADC provided herein comprises the heavy chain CDR 1 (CDR-H1), CDR-H2, and/or CDR-H3 of a VH amino acid sequence set forth in any one of SEQ ID NOs: 16, 18, and 60 as determined by the Kabat numbering scheme.

In certain embodiments, an antigen-binding domain of a bispecific antibody of an ADC provided herein comprises the CDR-L1, CDR-L2, and/or CDR-L3 of a VL amino acid sequence set forth in any one of SEQ ID NOs: 15, 17, and 59 as determined by the Chothia numbering scheme. In certain embodiments, an antigen-binding domain of a bispecific antibody of an ADC provided herein comprises the CDR-H1, CDR-H2, and/or CDR-H3 of a VH amino acid sequence set forth in any one of SEQ ID NOs: 16, 18, and 60 as determined by the Chothia numbering scheme.

In certain embodiments, an antigen-binding domain of a bispecific antibody of an ADC provided herein comprises the CDR-L1, CDR-L2, and/or CDR-L3 of a VL amino acid sequence set forth in any one of SEQ ID NOs: 15, 17, and 59 as determined by the MacCallum numbering scheme. In certain embodiments, an antigen-binding domain of a bispecific antibody of an ADC provided herein comprises the CDR-H1, CDR-H2, and/or CDR-H3 of a VH amino acid sequence set forth in any one of SEQ ID NOs: 16, 18, and 60 as determined by the MacCallum numbering scheme.

In certain embodiments, an antigen-binding domain of a bispecific antibody of an ADC provided herein comprises the CDR-L1, CDR-L2, and/or CDR-L3 of a VL amino acid sequence set forth in any one of SEQ ID NOs: 15, 17, and 59 as determined by the IMGT numbering scheme. In certain embodiments, an antigen-binding domain of a bispecific antibody of an ADC provided herein comprises the CDR-H1, CDR-H2, and/or CDR-H3 of a VH amino acid sequence set forth in any one of SEQ ID NOs: 16, 18, and 60 as determined by the IMGT numbering scheme.

In certain embodiments, an antigen-binding domain of a bispecific antibody of an ADC provided herein comprises the CDR-L1, CDR-L2, and/or CDR-L3 of a VL amino acid sequence set forth in any one of SEQ ID NOs: 15, 17, and 59 as determined by the AbM numbering scheme. In certain embodiments, an antigen-binding domain of a bispecific antibody of an ADC provided herein comprises the CDR-H1, CDR-H2, and/or CDR-H3 of a VH amino acid sequence set forth in any one of SEQ ID NOs: 16, 18, and 60 as determined by the AbM numbering scheme.

In certain embodiments, an antigen-binding domain of a bispecific antibody of an ADC provided herein comprises the CDR-L1, CDR-L2, and/or CDR-L3 of a VL amino acid sequence set forth in any one of SEQ ID NOs: 15, 17, and 59 as determined by the AHo numbering scheme. In certain embodiments, an antigen-binding domain of a bispecific antibody of an ADC provided herein comprises the CDR-H1, CDR-H2, and/or CDR-H3 of a VH amino acid sequence set forth in any one of SEQ ID NOs: 16, 18, and 60 as determined by the AHo numbering scheme.

In certain embodiments, the individual CDRs of an antigen-binding domain of a bispecific antibody of an ADC provided herein are each independently determined according to one of the Kabat, Chothia, MacCallum, IMGT, AHo, or AbM numbering schemes, or by structural analysis of the antigen-binding domain, wherein the structural analysis identifies residues in the variable region(s) predicted to make contact with an epitope region of c-MET or EGFR.

In certain embodiments, the instant disclosure provides an ADC comprising a bispecific antibody comprising a first antigen-binding domain specifically binding to c-MET and a second antigen-binding domain specifically binding to EGFR, wherein the first antigen-binding domain comprises a VL comprising the CDR-L1, CDR-L2, and CDR-L3 amino acid sequences of a VL amino acid sequence set forth in SEQ ID NO: 17 or 59, and a VH comprising the CDR-H1, CDR-H2, and CDR-H3 amino acid sequences of a VH amino acid sequence set forth in SEQ ID NO: 18 or 60 and/or wherein the second antigen-binding domain comprises a VL comprising the CDR-L1, CDR-L2, and CDR-L3 amino acid sequences of a VL amino acid sequence set forth in SEQ ID NO: 15, and a VH comprising the CDR-H1, CDR-H2, and CDR-H3 amino acid sequences of a VH amino acid sequence set forth in SEQ ID NO: 16, wherein each CDR is independently determined according to one of the Kabat, Chothia, MacCallum, IMGT, AHo, or AbM numbering schemes, or by structural analysis of the bispecific molecule, wherein the structural analysis identifies residues in the variable region(s) predicted to make contact with an epitope region of c-MET or EGFR.

In certain embodiments, the instant disclosure provides an ADC comprising a bispecific antibody comprising a first antigen-binding domain specifically binding to c-MET and a second antigen-binding domain specifically binding to EGFR, wherein the first antigen-binding domain comprises a VL comprising the CDR-L1, CDR-L2, and CDR-L3 amino acid sequences of a VL amino acid sequence set forth in SEQ ID NO: 17, and a VH comprising the CDR-H1, CDR-H2, and CDR-H3 amino acid sequences of a VH amino acid sequence set forth in SEQ ID NO: 18 and/or wherein the second antigen-binding domain comprises a VL comprising the CDR-L1, CDR-L2, and CDR-L3 amino acid sequences of a VL amino acid sequence set forth in SEQ ID NO: 15, and a VH comprising the CDR-H1, CDR-H2, and CDR-H3 amino acid sequences of a VH amino acid sequence set forth in SEQ ID NO: 16, wherein each CDR is independently determined according to one of the Kabat, Chothia, MacCallum, IMGT, Aho, or AbM numbering schemes, or by structural analysis of the bispecific molecule, wherein the structural analysis identifies residues in the variable region(s) predicted to make contact with an epitope region of c-MET or EGFR.

In certain embodiments, the instant disclosure provides an ADC comprising a bispecific antibody comprising a first antigen-binding domain specifically binding to c-MET and a second antigen-binding domain specifically binding to EGFR, wherein the first antigen-binding domain comprises a VL comprising the CDR-L1, CDR-L2, and CDR-L3 amino acid sequences of a VL amino acid sequence set forth in SEQ ID NO: 59, and a VH comprising the CDR-H1, CDR-H2, and CDR-H3 amino acid sequences of a VH amino acid sequence set forth in SEQ ID NO: 60 and/or wherein the second antigen-binding domain comprises a VL comprising the CDR-L1, CDR-L2, and CDR-L3 amino acid sequences of a VL amino acid sequence set forth in SEQ ID NO: 15, and a VH comprising the CDR-H1, CDR-H2, and CDR-H3 amino acid sequences of a VH amino acid sequence set forth in SEQ ID NO: 16, wherein each CDR is independently determined according to one of the Kabat, Chothia, MacCallum, IMGT, AHo, or AbM numbering schemes, or by structural analysis of the bispecific molecule, wherein the structural analysis identifies residues in the variable region(s) predicted to make contact with an epitope region of c-MET or EGFR.

In certain embodiments, the instant disclosure provides an ADC comprising a bispecific antibody comprising a first antigen-binding domain specifically binding to c-MET and a second antigen-binding domain specifically binding to EGFR, wherein the first antigen-binding domain comprises a VL comprising the CDR-L1, CDR-L2, and CDR-L3 amino acid sequences of a VL amino acid sequence set forth in SEQ ID NO: 17, and a VH comprising the CDR-H1, CDR-H2, and CDR-H3 amino acid sequences of a VH amino acid sequence set forth in SEQ ID NO: 60 and/or wherein the second antigen-binding domain comprises a VL comprising the CDR-L1, CDR-L2, and CDR-L3 amino acid sequences of a VL amino acid sequence set forth in SEQ ID NO: 15, and a VH comprising the CDR-H1, CDR-H2, and CDR-H3 amino acid sequences of a VH amino acid sequence set forth in SEQ ID NO: 16, wherein each CDR is independently determined according to one of the Kabat, Chothia, MacCallum, IMGT, AHo, or AbM numbering schemes, or by structural analysis of the bispecific molecule, wherein the structural analysis identifies residues in the variable region(s) predicted to make contact with an epitope region of c-MET or EGFR.

In certain embodiments, the instant disclosure provides an ADC comprising a bispecific antibody comprising a first antigen-binding domain specifically binding to c-MET and a second antigen-binding domain specifically binding to EGFR, wherein the first antigen-binding domain comprises a VL comprising the CDR-L1, CDR-L2, and CDR-L3 amino acid sequences of a VL amino acid sequence set forth in SEQ ID NO: 59, and a VH comprising the CDR-H1, CDR-H2, and CDR-H3 amino acid sequences of a VH amino acid sequence set forth in SEQ ID NO: 18 and/or wherein the second antigen-binding domain comprises a VL comprising the CDR-L1, CDR-L2, and CDR-L3 amino acid sequences of a VL amino acid sequence set forth in SEQ ID NO: 15, and a VH comprising the CDR-H1, CDR-H2, and CDR-H3 amino acid sequences of a VH amino acid sequence set forth in SEQ ID NO: 16, wherein each CDR is independently determined according to one of the Kabat, Chothia, MacCallum, IMGT, AHo, or AbM numbering schemes, or by structural analysis of the bispecific molecule, wherein the structural analysis identifies residues in the variable region(s) predicted to make contact with an epitope region of c-MET or EGFR.

In certain embodiments of the ADCs provided herein, the first VL of the bispecific antibody of the ADC comprises:
a CDR-L1 comprising the amino acid sequence shown in SEQ ID NO: 34, a CDR-L2 comprising the amino acid sequence shown in SEQ ID NO: 36, and a CDR-L3 comprising the amino acid sequence shown in SEQ ID NO: 38; wherein the CDRs are defined by the Kabat numbering system;
a CDR-L1 comprising the amino acid sequence shown in SEQ ID NO: 34, a CDR-L2 comprising the amino acid sequence shown in SEQ ID NO: 36, and a CDR-L3 comprising the amino acid sequence shown in SEQ ID NO: 38; wherein the CDRs are defined by the Chothia numbering system;
a CDR-L1 comprising the amino acid sequence shown in SEQ ID NO: 34, a CDR-L2 comprising the amino acid sequence shown in SEQ ID NO: 36, and a CDR-L3 comprising the amino acid sequence shown in SEQ ID NO: 38; wherein the CDRs are defined by the Abm numbering system; or
a CDR-L1 comprising the amino acid sequence shown in SEQ ID NO: 35, a CDR-L2 comprising the amino acid sequence shown in SEQ ID NO: 37, and a CDR-L3 comprising the amino acid sequence shown in SEQ ID NO: 38; wherein the CDRs are defined by the IMGT numbering system;
and/or
the first VH of the bispecific antibody of the ADC comprises:
a CDR-H1 comprising the amino acid sequence shown in SEQ ID NO: 39, a CDR-H2 comprising the amino acid sequence shown in SEQ ID NO: 43, and a CDR-H3 comprising the amino acid sequence shown in SEQ ID NO: 47; wherein the CDRs are defined by the Kabat numbering system;
a CDR-H1 comprising the amino acid sequence shown in SEQ ID NO: 40, a CDR-H2 comprising the amino acid sequence shown in SEQ ID NO: 44, and a CDR-H3 comprising the amino acid sequence shown in SEQ ID NO: 47; wherein the CDRs are defined by the Chothia numbering system;
a CDR-H1 comprising the amino acid sequence shown in SEQ ID NO: 42, a CDR-H2 comprising the amino acid sequence shown in SEQ ID NO: 46, and a CDR-H3 comprising the amino acid sequence shown in SEQ ID NO: 47; wherein the CDRs are defined by the Abm numbering system; or
a CDR-H1 comprising the amino acid sequence shown in SEQ ID NO: 41, a CDR-H2 comprising the amino acid sequence shown in SEQ ID NO: 45, and a CDR-H3 comprising the amino acid sequence shown in SEQ ID NO: 48; where the CDRs are defined by the IMGT numbering system.

In certain embodiments, the first VL comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 17 or 59. In certain embodiments, the first VL comprises an amino acid sequence set forth in SEQ ID NO: 17 or 59. In certain embodiments, the first VL consists of the amino acid sequence set forth in SEQ ID NO: 17 or 59.

In certain embodiments, the first VH comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 18 or 60. In certain embodiments, the first VH comprises an amino acid sequence set forth in SEQ ID NO: 18 or 60. In certain embodiments, the first VH consists of the amino acid sequence set forth in SEQ ID NO: 18 or 60.

In certain embodiments, the first VL comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 17 or 59 and the first VH comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 18 or 60. In certain embodiments, the first VL comprises an amino acid sequence set forth in SEQ ID NO: 17 or 59 and the first VH comprises an amino acid sequence set forth in SEQ ID NO: 18 or 60. In certain embodiments, the first VL consists of the amino acid sequence set forth in SEQ ID NO: 17 or 59 and the first VH consists of the amino acid sequence set forth in SEQ ID NO: 18 or 60.

In certain embodiments, the first VL comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 17 and the first VH comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 18. In certain embodiments, the first VL comprises an amino acid sequence set forth in SEQ ID NO: 17 and the first VH comprises an amino acid sequence set forth in SEQ ID NO: 18. In certain embodiments, the first VL consists of the amino acid sequence set forth in SEQ ID NO: 17 and the first VH consists of the amino acid sequence set forth in SEQ ID NO: 18.

In certain embodiments, the first VL comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 59 and the first VH comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 60. In certain embodiments, the first VL comprises an amino acid sequence set forth in SEQ ID NO: 59 and the first VH comprises an amino acid sequence set forth in SEQ ID NO: 60. In certain embodiments, the first VL consists of the amino acid sequence set forth in SEQ ID NO: 59 and the first VH consists of the amino acid sequence set forth in SEQ ID NO: 60.

In certain embodiments, the first VL comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 17 and the first VH comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 60. In certain embodiments, the first VL comprises an amino acid sequence set forth in SEQ ID NO: 17 and the first VH comprises an amino acid sequence set forth in SEQ ID NO: 60. In certain embodiments, the first VL consists of the amino acid sequence set forth in SEQ ID NO: 17 and the first VH consists of the amino acid sequence set forth in SEQ ID NO: 60.

In certain embodiments, the first VL comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 59 and the first VH comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 18. In certain embodiments, the first VL comprises an amino acid sequence set forth in SEQ ID NO: 59 and the first VH comprises an amino acid sequence set forth in SEQ ID NO: 18. In certain embodiments, the first VL consists of the amino acid sequence set forth in SEQ ID NO: 59 and the first VH consists of the amino acid sequence set forth in SEQ ID NO: 18.

In certain embodiments of the ADCs provided herein, the second VL of the bispecific antibody of the ADC comprises:
(i) a CDR-L1 comprising the amino acid sequence shown in SEQ ID NO: 19, a CDR-L2 comprising the amino acid sequence shown in SEQ ID NO: 21, and a CDR-L3 comprising the amino acid sequence shown in SEQ ID NO: 23; wherein the CDRs are defined by the Kabat numbering system;
(ii) a CDR-L1 comprising the amino acid sequence shown in SEQ ID NO: 19, a CDR-L2 comprising the amino acid sequence shown in SEQ ID NO: 21, and a CDR-L3 comprising the amino acid sequence shown in SEQ ID NO: 23; wherein the CDRs are defined by the Chothia numbering system;
(iii) a CDR-L1 comprising the amino acid sequence shown in SEQ ID NO: 19, a CDR-L2 comprising the amino acid sequence shown in SEQ ID NO: 21, and a CDR-L3 comprising the amino acid sequence shown in SEQ ID NO: 23; wherein the CDRs are defined by the Abm numbering system; or
(iv) a CDR-L1 comprising the amino acid sequence shown in SEQ ID NO: 20, a CDR-L2 comprising the amino acid sequence shown is SEQ ID NO: 22, and a CDR-L3 comprising the amino acid sequence shown in SEQ ID NO: 23; wherein the CDRs are defined by the IMGT numbering system;
and/or
the second VH of the bispecific antibody of the ADC comprises:
(i) a CDR-H1 comprising the amino acid sequence shown in SEQ ID NO: 24, a CDR-H2 comprising the amino acid sequence shown in SEQ ID NO: 28, and a CDR-H3 comprising the amino acid sequence shown in SEQ ID NO: 32; wherein the CDRs are defined by the Kabat numbering system;
(ii) a CDR-H1 comprising the amino acid sequence shown in SEQ ID NO: 25, a CDR-H2 comprising the amino acid sequence shown in SEQ ID NO: 29, and a CDR-H3 comprising the amino acid sequence shown in SEQ ID NO: 32; wherein the CDRs are defined by the Chothia numbering system;
(iii) a CDR-H1 comprising the amino acid sequence shown in SEQ ID NO: 27, a CDR-H2 comprising the amino acid sequence shown in SEQ ID NO: 31, and a CDR-H3 comprising the amino acid sequence shown in SEQ ID NO: 32; wherein the CDRs are defined by the Abm numbering system; or
(iv) a CDR-H1 comprising the amino acid sequence shown in SEQ ID NO: 26, a CDR-H2 comprising the amino acid sequence shown as SEQ ID NO: 30, and a CDR-H3 comprising the amino acid sequence shown in SEQ ID NO: 33; where the CDRs are defined by the IMGT numbering system.

In certain embodiments, the second VL comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 15. In certain embodiments, the second VL comprises an amino acid sequence set forth in SEQ ID NO: 15. In certain embodiments, the second VL consists of the amino acid sequence set forth in SEQ ID NO: 15.

In certain embodiments, the second VH comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 16. In certain embodiments, the second VH comprises an amino acid sequence set forth in SEQ ID NO: 16. In certain embodiments, the second VH consists of the amino acid sequence set forth in SEQ ID NO: 16.

In certain embodiments, the second VL comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 15 and the second VH comprises an amino acid sequence that is at least 75%, 80%, 85%, 90%, 95%, or 100% (e.g., at least 86%, 87%, 88%, 89%, 90%, 91%, 92%, 93%, 94%, 95%, 96%, 97%, 98%, or 99%) identical to the amino acid sequence set forth in SEQ ID NO: 16. In certain embodiments, the second VL comprises an amino acid sequence set forth in SEQ ID NO: 15 and the second VH comprises an amino acid sequence set forth in SEQ ID NO: 16. In certain embodiments, the second VL consists of the amino acid sequence set forth in SEQ ID NO: 15 and the second VH consists of the amino acid sequence set forth in SEQ ID NO: 16.

In certain embodiments, the instant disclosure provides an ADC comprising a bispecific antibody that cross-competes for binding to c-MET and/or EGFR with any of the antibodies described herein. In certain embodiments, the instant disclosure provides an ADC comprising a bispecific antibody that binds to the same or an overlapping epitope of c-MET and/or EGFR as an antibody described herein.

In certain embodiments, the epitope of an antibody can be determined by, e.g., NMR spectroscopy, surface plasmon resonance (BIAcore®), X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giege R et al., (1994) Acta Crystallogr D Biol Crystallogr 50 (Pt 4): 339-350; McPherson A (1990) Eur J Biochem 189: 1-23; Chayen N E (1997) Structure 5: 1269-1274; McPherson A (1976) J Biol Chem 251: 6300-6303, all of which are herein incorporated by reference in their entireties). Antibody:antigen crystals may be studied using well known X-ray diffraction techniques and may be refined using computer software such as X-PLOR (Yale University, 1992, distributed by Molecular Simulations, Inc.; see, e.g., Meth Enzymol (1985) volumes 114 & 115, eds. Wyckoff H W et al.; U.S. Patent Application No. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323, all of which are herein incorporated by reference in their entireties). Mutagenesis mapping studies may be accomplished using any method known to one of skill in the art. See, e.g., Champe M et al., (1995) and Cunningham B C & Wells J A (1989) for a description of mutagenesis techniques, including alanine scanning mutagenesis techniques. In a specific embodiment, the epitope of an antibody is determined using alanine scanning mutagenesis studies. In addition, bispecific antibodies that recognize and bind to the same or overlapping epitopes of c-MET and/or EGFR can be identified using routine techniques such as an immunoassay, for example, by showing the ability of one antibody to block the binding of another antibody to a target antigen, i.e., a competitive binding assay. Competition binding assays also can be used to determine whether two antibodies have similar binding specificity for an epitope. Competitive binding can be determined in an assay in which the immunoglobulin under test inhibits specific binding of a reference antibody to a common antigen, such as c-MET or EGFR. Numerous types of competitive binding assays are known, for example: solid phase direct or indirect radioimmunoassay (RIA), solid phase direct or indirect enzyme immunoassay (EIA), sandwich competition assay (see Stahli C et al., (1983) Methods Enzymol 9: 242-253); solid phase direct biotin-avidin EIA (see Kirkland T N et al., (1986) J Immunol 137: 3614-9); solid phase direct labeled assay, solid phase direct labeled sandwich assay (see Harlow E & Lane D, (1988) Antibodies: A Laboratory Manual, Cold Spring Harbor Press); solid phase direct label RIA using I-125 label (see Morel G A et al., (1988) Mol Immunol 25(1): 7-15); solid phase direct biotin-avidin EIA (see Cheung R C et al., (1990) Virology 176: 546-52); and direct labeled RIA (see Moldenhauer G et al., (1990) Scand J Immunol 32: 77-82), all of which are herein incorporated by reference in their entireties. Typically, such an assay involves the use of purified antigen (e.g., c-MET or EGFR) bound to a solid surface or cells bearing either of these, an unlabeled test immunoglobulin and a labeled reference immunoglobulin. Competitive inhibition can be measured by determining the amount of label bound to the solid surface or cells in the presence of the test immunoglobulin. Usually, the test immunoglobulin is present in excess. Usually, when a competing antibody is present in excess, it will inhibit specific binding of a reference or antibody to a common antigen by at least 50-55%, 55-60%, 60-65%, 65-70%, 70-75%, or more. A competition binding assay can be configured in a large number of different formats using either labeled antigen or labeled antibody. In a common version of this assay, the antigen is immobilized on a 96-well plate. The ability of unlabeled antibodies to block the binding of labeled antibodies to the antigen is then measured using radioactive or enzyme labels. For further details see, e.g., Wagener C et al., (1983) J Immunol 130: 2308-2315; Wagener C et al., (1984) J Immunol Methods 68: 269-274; Kuroki M et al., (1990) Cancer Res 50: 4872-4879; Kuroki M et al., (1992) Immunol Invest 21: 523-538; Kuroki M et al., (1992) Hybridoma 11: 391-407 and Antibodies: A Laboratory Manual, Ed Harlow E & Lane D editors supra, pp. 386-389, all of which are herein incorporated by reference in their entireties.

In certain embodiments of the ADCs provided herein, one, two, or more mutations (e.g., amino acid substitutions) are introduced into an Fc domain (e.g., a CH2 domain (residues 231-340 of human IgG1)) and/or a CH3 domain (residues 341-447 of human IgG1, numbered according to the EU numbering system) and/or a hinge region (residues 216-230, numbered according to the EU numbering system) of a bispecific antibody of the ADC, to alter one or more functional properties of the antibody, such as serum half-life, complement fixation, Fc receptor binding, and/or antigen-dependent cellular cytotoxicity.

In certain embodiments, one, two, or more mutations (e.g., amino acid substitutions) are introduced into an Fc domain to, e.g., reduce or ablate effector function of the Fc domain. In certain embodiments, the antibodies comprise an Fc domain comprising: (i) E233A and L235A (EALA) amino acid substitutions, wherein the numbering is according to the Eu numbering scheme; (ii) L234A and L235A (LALA) amino acid substitutions, wherein the numbering is according to the Eu numbering scheme; (iii) L234A L235A D265S (LALADS) amino acid substitutions, wherein the numbering is according to the Eu numbering scheme; (iv) L234A L235A P329G (LALAPG) amino acid substitutions, wherein the numbering is according to the Eu numbering scheme; (v) L234A L235A P329A (LALAPA) amino acid substitutions, wherein the numbering is according to the Eu numbering scheme; (vi) L235E (LE) amino acid substitutions, wherein the numbering is according to the Eu numbering scheme; (vii) D265A (DS) amino acid substitution, wherein the numbering is according to the Eu numbering scheme; (viii) D265A N297G (DANG) amino acid substitutions, wherein the numbering is according to the Eu numbering scheme; (ix) N297X amino acid substitution, wherein X is any amino acid other than N, wherein the numbering is according to the Eu numbering scheme; (x) N297A/D356E/L358M (NADELM) amino acid substitutions, wherein the numbering is according to Eu numbering scheme; or (x1) D356E L358M (DELM) amino acid substitutions, wherein the numbering is according to Eu numbering scheme.

In certain embodiments, any of the constant region mutations or modifications described herein can be introduced into one or both heavy chain constant regions of an antibody described herein having two heavy chain constant regions.

### Fab-scFv Structure

In certain embodiments, the first antigen-binding domain is a Fab and the second antigen-binding domain is an scFv.

In certain embodiments, the bispecific antibody comprises polypeptide chain I-A, polypeptide chain I-B, and polypeptide chain I-C; wherein (a) the elements comprised in polypeptide chain I-A comprise a first VL and a first CL; the elements comprised in polypeptide chain I-B comprise a first VH, a first heavy chain CH1 region, and a first Fc domain monomer; and the elements comprised in polypeptide chain I-C comprise a second VL, a second VH, a second heavy chain CH1 hinge region, and a second Fc domain monomer; or (b) the elements comprised in polypeptide chain I-A comprise a first VL and a first CL; the elements comprised in polypeptide chain I-B comprise a first VH, a first heavy chain CH1 region, and a second Fc domain monomer; and the elements comprised in polypeptide chain I-C comprise a second VL, a second VH, a second heavy chain CH1 hinge region, and a first Fc domain monomer.

In certain embodiments, (a) the elements comprised in polypeptide chain I-A comprise from N-terminus to C-terminus the first VL and the first CL; the elements comprised in polypeptide chain I-B comprise from N-terminus to C-terminus the first VH, the first heavy chain CH1 region, and the first Fc domain monomer; and/or the elements comprised in polypeptide chain I-C comprise from N-terminus to C-terminus (i) the second VL, the second VH, the second heavy chain CH1 hinge region, and the second Fc domain monomer, or (ii) the second VH, the second VL, the second heavy chain CH1 hinge region, and the second Fc domain monomer; or (b) the elements comprised in polypeptide chain I-A comprise from N-terminus to C-terminus the first VL and the first CL; the elements comprised in polypeptide chain I-B comprise from N-terminus to C-terminus the first VH, the first heavy chain CH1, and the second Fc domain monomer; and/or the elements comprised in polypeptide chain I-C comprise from N-terminus to C-terminus: (i) the second VL, the second VH, the second heavy chain CH1 hinge region, and the first Fc domain monomer, or (ii) the second VH, the second VL, the second heavy chain CH1 hinge region, and the first Fc domain monomer.

In certain embodiments, the adjacent elements comprised in the polypeptide chain I-A are optionally connected to each other by a peptide linker or not; the adjacent elements comprised in the polypeptide chain I-B are optionally connected to each other by a peptide linker or not; and/or the adjacent elements comprised in the polypeptide chain I-C are optionally connected by a peptide linker or not.

In certain embodiments, the peptide linkers are each independently the same peptide linker or a different peptide linker (e.g., a rigid peptide linker or a flexible peptide linker). In certain embodiments, each of the peptide linkers independently comprises 3 to 55 (e.g., 3-10, 10-25, 10-20, 10 to 17, 10 to 15, 10-25, 10-35, 10-45, 10-55) amino acid residues. In certain embodiments, the peptide linkers are each independently selected from peptide linkers comprising one or more glycine (G) and/or serine (S) residues. In certain embodiments, each peptide linker may independently comprise 1, 2, 3, 4, 5, 6, 7, 8, 9, or 10 glycine residues. In certain embodiments, each peptide linker may independently comprise the peptide GGGG (SEQ ID NO: 61). In certain embodiments, the peptide linkers each independently comprise 1, 2, 3, 4, 5, 6, 7, 8, 9 or 10 tandem copies of the peptide linker subunit GGGGS (SEQ ID NO: 57), which may be represented by (GGGGS)n, wherein n is selected from 1, 2, 3, 4, 5, 6, 7, 8, 9 or 10 (SEQ ID NOs: 57, 65, 55, 58, 66-68, 56, 69, or 70, respectively). In certain embodiments, the peptide linkers each independently comprise the amino acid sequence shown in any one of SEQ ID NOs: 55-58 or 65-70.

In certain embodiments, the first CL comprises the amino acid sequence shown in SEQ ID NO: 53 or SEQ ID NO: 63. In certain embodiments, the first heavy chain CH1 region comprises the amino acid sequence shown in SEQ ID NO: 54. In certain embodiments, the second heavy chain CH1 hinge region comprises the amino acid sequence shown in SEQ ID NO: 62.

In certain embodiments, the first VH comprises the amino acid sequence shown in SEQ ID NO: 18 and the first VL comprises the amino acid sequence shown in SEQ ID NO: 17. In certain embodiments, the first VH comprises the amino acid sequence shown in SEQ ID NO: 60 and the first VL comprises the amino acid sequence shown in SEQ ID NO: 59. In certain embodiments, the second VH comprises the amino acid sequence shown in SEQ ID NO: 16 and the second VL comprises the amino acid sequence shown in SEQ ID NO: 15. In certain embodiments, the first VH comprises the amino acid sequence shown in SEQ ID NO: 18, the first VL comprises the amino acid sequence shown in SEQ ID NO: 17, the second VH comprises the amino acid sequence shown in SEQ ID NO: 16, and the second VL comprises the amino acid sequence shown in SEQ ID NO: 15. In certain embodiments, the first VH comprises the amino acid sequence shown in SEQ ID NO: 60, the first VL comprises the amino acid sequence shown in SEQ ID NO: 59, the second VH comprises the amino acid sequence shown in SEQ ID NO: 16, and the second VL comprises the amino acid sequence shown in SEQ ID NO: 15.

In certain embodiments, the first Fc domain monomer comprises the amino acid sequence shown in SEQ ID NO: 49 and the second Fc domain monomer comprises the amino acid sequence shown in SEQ ID NO: 50. In certain embodiments, the first Fc domain monomer comprises the amino acid sequence shown in SEQ ID NO: 51 and the second Fc domain monomer comprises the amino acid sequence shown in SEQ ID NO: 52.

In certain embodiments, the first Fc domain monomer comprises the amino acid sequence shown in SEQ ID NO: 50 and the second Fc domain monomer comprises the amino acid sequence shown in SEQ ID NO: 49. In certain embodiments, the first Fc domain monomer comprises the amino acid sequence shown in SEQ ID NO: 52 and the second Fc domain monomer comprises the amino acid sequence shown in SEQ ID NO: 51.

In certain embodiments, the polypeptide chain I-A comprises the amino acid sequence shown in SEQ ID NO: 1, the polypeptide chain I-B comprises the amino acid sequence shown in SEQ ID NO: 2 or 9, and/or the polypeptide chain I-C comprises the amino acid sequence shown in SEQ ID NO: 3 or 10.

scFv-Fab Structure

In certain embodiments, the first antigen-binding domain is an scFv and the second antigen-binding domain is a Fab.

In certain embodiments, the bispecific antibody comprises polypeptide chain II-A, polypeptide chain II-B, and polypeptide chain II-C; wherein (a) the elements comprised in polypeptide chain II-A comprise a second VL and a second CL; the elements comprised in polypeptide chain II-B comprise a second VH, a second heavy chain CH1 region, and a first Fc domain monomer; the elements comprised in polypeptide chain II-C comprise a first VL, a first VH, a first heavy chain CH1 hinge region, and a second Fc domain monomer, or (b) the elements comprised in polypeptide chain II-A comprise a second VL and a second CL; the elements comprised in polypeptide chain II-B comprise a second VH, a second heavy chain CH1 region, and a second Fc domain monomer; and the elements comprised in polypeptide chain II-C comprise a first VL, a first VH, a first heavy chain CH1 hinge region, and a first Fc domain monomer.

In certain embodiments, (a) the elements comprised in the polypeptide chain II-A comprise from N-terminus to C-terminus the second VL and the second CL; the elements comprised in polypeptide chain II-B comprise from N-terminus to C-terminus the second VH, the second heavy chain CH1 region, and the first Fc domain monomer; and/or the elements comprised in polypeptide chain II-C comprise from N-terminus to C-terminus: (i) the first VL, the first VH, the first heavy chain CH1 hinge region, and the second Fc domain monomer, or (ii) the first VH, the first VL, the first heavy chain CH1 hinge region, and the second Fc domain monomer, or (b) the elements comprised in polypeptide chain II-A comprise from N-terminus to C-terminus the second VL and the second CL; the elements comprised in polypeptide chain II-B comprise from N-terminus to C-terminus the second VH, the second heavy chain CH1 region, and the second Fc domain monomer; and/or the elements comprised in polypeptide chain II-C comprise from N-terminus to C-terminus: (i) the first VL, the first VH, the first heavy chain CH1 hinge region, and the first Fc domain monomer, or (ii) the first VH, the first VL, the first heavy chain CH1 hinge region, and the first Fc domain monomer.

In certain embodiments, the adjacent elements comprised in the polypeptide chain II-A are optionally connected to each other by a peptide linker or not; the adjacent elements comprised in the polypeptide chain II-B are optionally connected to each other by a peptide linker or not; and/or the adjacent elements comprised in the polypeptide chain II-C are optionally connected to each other by a peptide linker or not.

In certain embodiments, the peptide linkers are each independently the same peptide linker or a different peptide linker (e.g., a rigid peptide linker or a flexible peptide linker). In certain embodiments, each of the peptide linkers independently comprises 3 to 55 (e.g., 3-10, 10-25, 10-20, 10 to 17, 10 to 15, 10-25, 10-35, 10-45, 10-55) amino acid residues. In certain embodiments, the peptide linkers are each independently selected from peptide linkers comprising one or more glycine (G) and/or serine (S) residues. In certain embodiments, the peptide linkers each independently comprise 1, 2, 3, 4, 5, 6, 7, 8, 9 or 10 tandem copies of the peptide linker subunit GGGGS (SEQ ID NO: 57), which may be represented by (GGGGS)ₙ, wherein n is selected from 1, 2, 3, 4, 5, 6, 7, 8, 9 or 10 (SEQ ID NOs: 57, 65, 55, 58, 66-68, 56, 69, or 70, respectively). In certain embodiments, the peptide linkers each independently comprise the amino acid sequence shown in any one of SEQ ID NOs: 55-58 or 65-70.

In certain embodiments, the second CL comprises the amino acid sequence shown in SEQ ID NO: 53 or SEQ ID NO: 63, the first and second heavy chain CH1 region comprises the amino acid sequence shown in SEQ ID NO: 54, and/or the second heavy chain CH1 hinge region comprises the amino acid sequence shown in SEQ ID NO: 62.

In certain embodiments, the first VH comprises the amino acid sequence shown in SEQ ID NO: 18 and the first VL comprises the amino acid sequence shown in SEQ ID NO: 17. In certain embodiments, the first VH comprises the amino acid sequence shown in SEQ ID NO: 60 and the first VL comprises the amino acid sequence shown in SEQ ID NO: 59. In certain embodiments, the second VH comprises the amino acid sequence shown in SEQ ID NO: 16 and the second VL comprises the amino acid sequence shown in SEQ ID NO: 15. In certain embodiments, the first VH comprises the amino acid sequence shown in SEQ ID NO: 18, the first VL comprises the amino acid sequence shown in SEQ ID NO: 17, the second VH comprises the amino acid sequence shown in SEQ ID NO: 16, and the second VL comprises the amino acid sequence shown in SEQ ID NO: 15. In certain embodiments, the first VH comprises the amino acid sequence shown in SEQ ID NO: 60, the first VL comprises the amino acid sequence shown in SEQ ID NO: 59, the second VH comprises the amino acid sequence shown in SEQ ID NO: 16, and the second VL comprises the amino acid sequence shown in SEQ ID NO: 15.

In certain embodiments, the first Fc domain monomer comprises the amino acid sequence shown in SEQ ID NO: 49 and the second Fc domain monomer comprises the amino acid sequence shown in SEQ ID NO: 50. In certain embodiments, the first Fc domain monomer comprises the amino acid sequence shown in SEQ ID NO: 51 and the second Fc domain monomer comprises the amino acid sequence shown in SEQ ID NO: 52.

In certain embodiments, the first Fc domain monomer comprises the amino acid sequence shown in SEQ ID NO: 50 and the second Fc domain monomer comprises the amino acid sequence shown in SEQ ID NO: 49. In certain embodiments, the first Fc domain monomer comprises the amino acid sequence shown in SEQ ID NO: 52 and the second Fc domain monomer comprises the amino acid sequence shown in SEQ ID NO: 51.

In certain embodiments, the polypeptide chain II-A comprises the amino acid sequence shown in SEQ ID NO: 4, the polypeptide chain II-B comprises the amino acid sequence shown in SEQ ID NO: 5 or 7, and/or the polypeptide chain II-C comprises the amino acid sequence shown in SEQ ID NO: 6 or 8.

### Fab-scFab Structure

In certain embodiments, the first antigen-binding domain is a Fab and the second antigen-binding domain is a scFab (single chain Fab).

In certain embodiments, the bispecific antibody comprises polypeptide chain III-A, polypeptide chain III-B, and polypeptide chain III-C; wherein (a) the elements comprised in polypeptide chain III-A comprise a first VL and a first CL; the elements comprised in polypeptide chain III-B comprise a first VH, a first heavy chain CH1 region, and a first Fc domain monomer; and the elements comprised in polypeptide chain III-C comprise a second VL, a second CL, a second VH, a second heavy chain CH1 region, and a second Fc domain monomer; or (b) the elements comprised in polypeptide chain III-A comprise a first VL and a first CL; the elements comprised in polypeptide chain III-B comprise a first VH, a first heavy chain CH1 region, and a second Fc domain monomer; and the elements comprised in polypeptide chain III-C comprise a second VL, a second CL, a second VH, a second heavy chain CH1 region, and a first Fc domain monomer.

In certain embodiments, (a) the elements comprised in polypeptide chain III-A comprise from N-terminus to C-terminus the first VL and the first CL; the elements comprised in polypeptide chain III-B comprise from N-terminus to C-terminus the first VH, the first heavy chain CH1 region, and the first Fc domain monomer; and/or the elements comprised in polypeptide chain III-C comprise from N-terminus to C-terminus: (i) the second VL, the second CL, the second VH, the second heavy chain CH1 region, and the second Fc domain monomer; or (ii) the second VH, the second heavy chain CH1 region, the second VL, the second CL, and the second Fc domain monomer; or (b) the elements comprised in polypeptide chain III-A comprise from N-terminus to C-terminus the first VL and the first CL; the elements comprised in polypeptide chain III-B comprise from N-terminus to C-terminus the first VH, the first heavy chain CH1 region, and the second Fc domain monomer; and/or the elements comprised in polypeptide chain III-C comprise from N-terminus to C-terminus: (i) the second VL, the second CL, the second VH, the second heavy chain CH1 region, and the first Fc domain monomer; or (ii) the second VH, the second heavy chain CH1 region, the second VL, the second CL, and the first Fc domain monomer.

In certain embodiments, the adjacent elements comprised in the polypeptide chain III-A are optionally connected to each other by a peptide linker or not; the adjacent elements comprised in the polypeptide chain III-B are optionally connected to each other by a peptide linker or not; and/or the adjacent elements comprised in the polypeptide chain III-C are optionally connected to each other by a peptide linker or not.

In certain embodiments, the peptide linkers are each independently the same peptide linker or a different peptide linker (e.g., a rigid peptide linker or a flexible peptide linker). In certain embodiments, each of the peptide linkers independently comprises 3 to 55 (e.g., 3-10, 10-25, 10-20, 10 to 17, 10 to 15, 10-25, 10-35, 10-45, 10-55) amino acid residues. In certain embodiments, the peptide linkers are each independently selected from peptide linkers comprising one or more glycine (G) and/or serine (S) residues. In certain embodiments, the peptide linkers each independently comprise 1, 2, 3, 4, 5, 6, 7, 8, 9 or 10 tandem copies of the peptide linker unit GGGGS (SEQ ID NO: 57), which may be represented by (GGGGS)ₙ, wherein n is selected from 1, 2, 3, 4, 5, 6, 7, 8, 9 or 10 (SEQ ID NOs: 57, 65, 55, 58, 66-68, 56, 69, or 70, respectively). In certain embodiments, the peptide linkers each independently comprise the amino acid sequence shown in any one of SEQ ID NOs: 55-58 or 65-70.

In certain embodiments, the first and second CL comprises the amino acid sequence shown in SEQ ID NO: 53 or SEQ ID NO: 63, the first and second heavy chain CH1 region comprises the amino acid sequence shown in SEQ ID NO: 54, and/or the second heavy chain CH1 hinge region comprises the amino acid sequence shown in SEQ ID NO: 62.

In certain embodiments, the first VH comprises the amino acid sequence shown in SEQ ID NO: 18 and the first VL comprises the amino acid sequence shown in SEQ ID NO: 17. In certain embodiments, the first VH comprises the amino acid sequence shown in SEQ ID NO: 60 and the first VL comprises the amino acid sequence shown in SEQ ID NO: 59. In certain embodiments, the second VH comprises the amino acid sequence shown in SEQ ID NO: 16 and the second VL comprises the amino acid sequence shown in SEQ ID NO: 15. In certain embodiments, the first VH comprises the amino acid sequence shown in SEQ ID NO: 18, the first VL comprises the amino acid sequence shown in SEQ ID NO: 17, the second VH comprises the amino acid sequence shown in SEQ ID NO: 16, and the second VL comprises the amino acid sequence shown in SEQ ID NO: 15. In certain embodiments, the first VH comprises the amino acid sequence shown in SEQ ID NO: 60, the first VL comprises the amino acid sequence shown in SEQ ID NO: 59, the second VH comprises the amino acid sequence shown in SEQ ID NO: 16, and the second VL comprises the amino acid sequence shown in SEQ ID NO: 15.

In certain embodiments, the first Fc domain monomer comprises the amino acid sequence shown in SEQ ID NO: 49 and the second Fc domain monomer comprises the amino acid sequence shown in SEQ ID NO: 50. In certain embodiments, the first Fc domain monomer comprises the amino acid sequence shown in SEQ ID NO: 51 and the second Fc domain monomer comprises the amino acid sequence shown in SEQ ID NO: 52.

In certain embodiments, the first Fc domain monomer comprises the amino acid sequence shown in SEQ ID NO: 50 and the second Fc domain monomer comprises the amino acid sequence shown in SEQ ID NO: 49. In certain embodiments, the first Fc domain monomer comprises the amino acid sequence shown in SEQ ID NO: 52 and the second Fc domain monomer comprises the amino acid sequence shown in SEQ ID NO: 51.

scFab-Fab Structure

In certain embodiments, the first antigen-binding domain is an scFab and the second antigen-binding domain is a Fab.

In certain embodiments, the bispecific antibody comprises polypeptide chain IV-A, polypeptide chain IV-B; and polypeptide chain IV-C, wherein (a) the elements comprised in polypeptide chain IV-A comprise a second VL and a second CL; the elements comprised in chain IV-B comprise a second VH, a second heavy chain CH1 region, and a first Fc domain monomer; and the elements comprised in polypeptide chain IV-C comprise a first VL, a first CL, a first VH, a first heavy chain CH1 region, and a second Fc domain monomer; or (b) the elements comprised in polypeptide chain IV-A comprise a second VL and a second CL; the elements comprised in polypeptide chain IV-B comprise a second VH, a second heavy chain CH1 region, and a second Fc domain monomer; and the elements comprised in polypeptide chain IV-C comprise a first VL, a first CL, a first VH, a first heavy chain CH1 region, and a first Fc domain monomer.

In certain embodiments, (a) the elements comprised in polypeptide chain IV-A comprise from N-terminus to C-terminus the second VL and the second CL; the elements comprised in polypeptide chain IV-B comprise from N-terminus to C-terminus the second VH, the second heavy chain CH1 region, and the first Fc domain monomer; and/or the elements comprised in polypeptide chain IV-C comprise from N-terminus to C-terminus the first VL, the first CL, the first VH, the first heavy chain CH1 region, and the second Fc domain monomer; or (b) the elements comprised in polypeptide chain IV-A comprise from N-terminus to C-terminus the second VL and the second CL; the elements comprised in polypeptide chain IV-B comprise from N-terminus to C-terminus the second VH, the second heavy chain CH1 region, and the second Fc domain monomer; and/or the elements comprised in polypeptide chain IV-C comprise from N-terminus to C-terminus (i) the first VL, the first CL, the first VH, the first heavy chain CH1 region, and the first Fc domain monomer.

In certain embodiments, the adjacent elements comprised in the polypeptide chain IV-A are optionally connected to each other by a peptide linker or not; the adjacent elements comprised in the polypeptide chain IV-B are optionally connected to each other by a peptide linker or not; and/or the adjacent elements comprised in the polypeptide chain IV-C are optionally connected to each other by a peptide linker or not.

In certain embodiments, the peptide linkers are each independently the same peptide linker or a different peptide linker (e.g., a rigid peptide linker or a flexible peptide linker). In certain embodiments, each of the peptide linkers independently comprises 3 to 55 (e.g., 3-10, 10-25, 10-20, 10 to 17, 10 to 15, 10-25, 10-35, 10-45, 10-55) amino acid residues. In certain embodiments, the peptide linkers are each independently selected from peptide linkers comprising one or more glycine (G) and/or serine (S) residues. In certain embodiments, the peptide linkers each independently comprise 1, 2, 3, 4, 5, 6, 7, 8, 9 or 10 tandem copies of the peptide linker subunit GGGGS (SEQ ID NO: 57), which may be represented by (GGGGS)ₙ, wherein n is selected from 1, 2, 3, 4, 5, 6, 7, 8, 9 or 10 (SEQ ID NOs: 57, 65, 55, 58, 66-68, 56, 69, or 70, respectively). In certain embodiments, the peptide linkers each independently comprise the amino acid sequence shown in any one of SEQ ID NOs: 55-58 or 65-70.

In certain embodiments, the first and second CL comprise the amino acid sequence shown in SEQ ID NO: 53 or SEQ ID NO: 63, the first and second heavy chain CH1 region comprises the amino acid sequence shown in SEQ ID NO: 54, and/or the first heavy chain CH1 hinge region comprises the amino acid sequence shown in SEQ ID NO: 62.

In certain embodiments, the first VH comprises the amino acid sequence shown in SEQ ID NO: 18 and the first VL comprises the amino acid sequence shown in SEQ ID NO: 17. In certain embodiments, the first VH comprises the amino acid sequence shown in SEQ ID NO: 60 and the first VL comprises the amino acid sequence shown in SEQ ID NO: 59. In certain embodiments, the first VL comprises the amino acid sequence shown in SEQ ID NO: 59 and the first VH comprises the amino acid sequence shown in SEQ ID NO: 60. In certain embodiments, the second VH comprises the amino acid sequence shown in SEQ ID NO: 16 and the second VL comprises the amino acid sequence shown in SEQ ID NO: 15. In certain embodiments, the first VH comprises the amino acid sequence shown in SEQ ID NO: 18, the first VL comprises the amino acid sequence shown in SEQ ID NO: 17, the second VH comprises the amino acid sequence shown in SEQ ID NO: 16, and the second VL comprises the amino acid sequence shown in SEQ ID NO: 15. In certain embodiments, the first VH comprises the amino acid sequence shown in SEQ ID NO: 60, the first VL comprises the amino acid sequence shown in SEQ ID NO: 59, the second VH comprises the amino acid sequence shown in SEQ ID NO: 16, and the second VL comprises the amino acid sequence shown in SEQ ID NO: 15.

In certain embodiments, the first Fc domain monomer comprises the amino acid sequence shown in SEQ ID NO: 49 and the second Fc domain monomer comprises the amino acid sequence shown in SEQ ID NO: 50. In certain embodiments, the first Fc domain monomer comprises the amino acid sequence shown in SEQ ID NO: 51 and the second Fc domain monomer comprises the amino acid sequence shown in SEQ ID NO: 52.

In certain embodiments, the first Fc domain monomer comprises the amino acid sequence shown in SEQ ID NO: 50 and the second Fc domain monomer comprises the amino acid sequence shown in SEQ ID NO: 49. In certain embodiments, the first Fc domain monomer comprises the amino acid sequence shown in SEQ ID NO: 52 and the second Fc domain monomer comprises the amino acid sequence shown in SEQ ID NO: 51.

In certain embodiments, the polypeptide chain IV-A comprises the amino acid sequence shown in SEQ ID NO: 4, the polypeptide chain IV-B comprises the amino acid sequence shown in SEQ ID NO: 7, and/or the polypeptide chain IV-C comprises the amino acid sequence shown in SEQ ID NO: 11.

### Fab-Fab (Domain Swapping Including CrossMab Form) Structures

In certain embodiments, the first antigen-binding domain and the second antigen-binding domain are Fabs, and the Fab of the second antigen-binding domain comprises a domain swap in the form of a CrossMab.

In certain embodiments, the Fab of the second antigen-binding domain comprises a domain swap of the VH and VL in the form of CrossMab^{VH-VL}.

In certain embodiments, the Fab of the second antigen-binding domain comprises a domain swap of the CH1 and CL in the form of CrossMab^{CH1-CL}.

In certain embodiments, the Fab of the second antigen-binding domain comprises a domain swap in the form of CrossMab^{Fab}.

In certain embodiments, the bispecific antibody comprising a domain swap in the form of CrossMab^{CH1-CL} comprises polypeptide chain V-A, polypeptide chain V-B, polypeptide chain V-C, and polypeptide chain V-D; wherein (a) the elements comprised in polypeptide chain V-A comprise a first VL and a first CL; the elements comprised in polypeptide chain V-B comprise a first VH, a first heavy chain CH1 region, and a first Fc domain monomer; the elements comprised in polypeptide chain V-C comprise a second VH, a second CL, a second heavy chain CH1 hinge region, and a second Fc domain monomer; and the elements comprised in polypeptide chain V-D comprise a second VL and a second heavy chain CH1 region; or (b) the elements comprised in polypeptide chain V-A comprise a first VL and a first CL; the elements comprised in polypeptide chain V-B comprise a first VH, a first heavy chain CH1 region, and a second Fc domain monomer; the elements comprised in polypeptide chain V-C comprise a second VH, a second CL, a second heavy chain CH1 hinge region, and a first Fc domain monomer; and the elements comprised in polypeptide chain V-D comprise a second VL and a second heavy chain CH1 region.

In certain embodiments, (a) the elements comprised in polypeptide chain V-A comprise from N-terminus to C-terminus the first VL and the first CL; the elements comprised in polypeptide chain V-B comprise from N-terminus to C-terminus the first VH, the first heavy chain CH1 region, and the first Fc domain monomer; the elements comprised in polypeptide chain V-C comprise from N-terminus to C-terminus the second VH, the second CL, the second heavy chain CH1 hinge region, and the second Fc domain monomer, and/or the elements comprised in polypeptide chain V-D comprise from N-terminus to C-terminus the second VL and the second heavy chain CH1 region; or (b) the elements comprised in polypeptide chain V-A comprise from N-terminus to C-terminus the first VL and the first CL; the elements comprised in polypeptide chain V-B comprise from N-terminus to C-terminus the first VH, the first heavy chain CH1 region, and the second Fc domain monomer; the elements comprised in polypeptide chain V-C comprise from N-terminus to C-terminus the second VH, the second CL, the second heavy chain CH1 hinge region, and the first Fc domain monomer, and/or the elements comprised in polypeptide chain V-D comprise from N-terminus to C-terminus the second VL and the second heavy chain CH1 region.

In certain embodiments, the adjacent elements comprised in the polypeptide chain V-A are optionally connected to each other by a peptide linker or not; the adjacent elements comprised in the polypeptide chain V-B are optionally connected to each other by a peptide linker or not; the adjacent elements comprised in the polypeptide chain V-C are optionally connected to each other by a peptide linker or not; and/or the adjacent elements comprised in the polypeptide chain V-D are optionally connected to each other by a peptide linker or not.

In certain embodiments, the peptide linkers are each independently the same peptide linker or a different peptide linker (e.g., a rigid peptide linker or a flexible peptide linker). In certain embodiments, each of the peptide linkers independently comprises 3 to 55 (e.g., 3-10, 10-25, 10-20, 10 to 17, 10 to 15, 10-25, 10-35, 10-45, 10-55) amino acid residues. In certain embodiments, the peptide linkers are each independently selected from peptide linkers comprising one or more glycine (G) and/or serine (S) residues. In certain embodiments, the peptide linkers each independently comprise 1, 2, 3, 4, 5, 6, 7, 8, 9 or 10 tandem copies of the peptide linker unit GGGGS (SEQ ID NO: 57), which may be represented by (GGGGS)ₙ, wherein n is selected from 1, 2, 3, 4, 5, 6, 7, 8, 9 or 10 (SEQ ID NOs: 57, 65, 55, 58, 66-68, 56, 69, or 70, respectively). In certain embodiments, the peptide linkers each independently comprise the amino acid sequence shown in any one of SEQ ID NOs: 55-58 or 65-70.

In certain embodiments, the first and second CL comprises the amino acid sequence shown in SEQ ID NO: 53 or SEQ ID NO: 63, the first and second heavy chain CH1 region comprises the amino acid sequence shown in SEQ ID NO: 54, and/or the second heavy chain CH1 hinge region comprises the amino acid sequence shown in SEQ ID NO: 62.

In certain embodiments, the first VH comprises the amino acid sequence shown in SEQ ID NO: 18 and the first VL comprises the amino acid sequence shown in SEQ ID NO: 17. In certain embodiments, the first VH comprises the amino acid sequence shown in SEQ ID NO: 60 and the first VL comprises the amino acid sequence shown in SEQ ID NO: 59. In certain embodiments, the second VH comprises the amino acid sequence shown in SEQ ID NO: 16 and the second VL comprises the amino acid sequence shown in SEQ ID NO: 15. In certain embodiments, the first VH comprises the amino acid sequence shown in SEQ ID NO: 18, the first VL comprises the amino acid sequence shown in SEQ ID NO: 17, the second VH comprises the amino acid sequence shown in SEQ ID NO: 16, and the second VL comprises the amino acid sequence shown in SEQ ID NO: 15. In certain embodiments, the first VH comprises the amino acid sequence shown in SEQ ID NO: 60, the first VL comprises the amino acid sequence shown in SEQ ID NO: 59, the second VH comprises the amino acid sequence shown in SEQ ID NO: 16, and the second VL comprises the amino acid sequence shown in SEQ ID NO: 15.

In certain embodiments, the first Fc domain monomer comprises the amino acid sequence shown in SEQ ID NO: 49 and the second Fc domain monomer comprises the amino acid sequence shown in SEQ ID NO: 50. In certain embodiments, the first Fc domain monomer comprises the amino acid sequence shown in SEQ ID NO: 51 and the second Fc domain monomer comprises the amino acid sequence shown in SEQ ID NO: 52.

In certain embodiments, the first Fc domain monomer comprises the amino acid sequence shown in SEQ ID NO: 50 and the second Fc domain monomer comprises the amino acid sequence shown in SEQ ID NO: 49. In certain embodiments, the first Fc domain monomer comprises the amino acid sequence shown in SEQ ID NO: 52 and the second Fc domain monomer comprises the amino acid sequence shown in SEQ ID NO: 51.

In certain embodiments, the polypeptide chain V-A comprises the amino acid sequence shown in SEQ ID NO: 1, the polypeptide chain V-B comprises the amino acid sequence shown in SEQ ID NO: 9, the polypeptide chain V-C comprises the amino acid sequence shown in SEQ ID NO: 13, and/or the polypeptide chain V-D comprises the amino acid sequence shown in SEQ ID NO: 12.

### Fab (Contains Domain Swapping in the Form of CrossMab)-Fab Structures

In certain embodiments, the first antigen-binding domain and the second antigen-binding domain are Fabs, and the Fab of the first antigen-binding domain comprises a domain swap in the form of a CrossMab.

In certain embodiments, the Fab of the first antigen-binding domain comprises a domain swap of the CH1 and CL in the form of CrossMab^{CH1-CL}.

In certain embodiments, the Fab of the first antigen-binding domain comprises a domain swap of the VH and VL in the form of CrossMab^{VH-VL}.

In certain embodiments, the Fab of the first antigen-binding domain comprises a domain swap in the form of CrossMab^{Fab}.

In certain embodiments, the bispecific antibody comprises polypeptide chain VI-A, polypeptide chain VI-B, polypeptide chain VI-C, and polypeptide chain VI-D; wherein (a) the elements comprised in polypeptide chain VI-A comprise a second VL and a second CL; the elements comprised in polypeptide chain VI-B comprise a second VH, a heavy chain CH1 region, and a first Fc domain monomer; the elements comprised in polypeptide chain VI-C comprise a first VH, a first CL, and a second Fc domain monomer; and the elements comprised in polypeptide chain VI-D comprise a first VL and a first heavy chain CH1 region; or (b) the elements comprised in polypeptide chain VI-A comprise a second VL and a second CL; the elements comprised in polypeptide chain VI-B comprise a second VH, a heavy chain CH1 region, and a second Fc domain monomer; the elements comprised in polypeptide chain VI-C comprise a first VH, a first CL, a first heavy chain CH1 hinge region, and a first Fc domain monomer; and the elements comprised in polypeptide chain VI-D comprise first VL and a first heavy chain CH1 region.

In certain embodiments, (a) the elements comprised in polypeptide chain VI-A comprise from N-terminus to C-terminus the second VL and the second CL; the elements comprised in polypeptide chain VI-B comprise from N-terminus to C-terminus the second VH, the heavy chain CH1 region, and the first Fc domain monomer; the elements comprised in polypeptide chain VI-C comprise from N-terminus to C-terminus the first VH, the first CL, the first heavy chain CH1 hinge region, and the second Fc domain monomer; and/or the elements comprised in polypeptide chain VI-D comprise from N-terminus to C-terminus the first VL and the heavy chain CH1 region; or (b) the elements comprised in polypeptide chain VI-A comprise from N-terminus to C-terminus the second VL and the second CL; the elements comprised in polypeptide chain VI-B comprise from N-terminus to C-terminus the second VH, the heavy chain CH1 region, and the second Fc domain monomer; the elements comprised in polypeptide chain VI-C comprise from N-terminus to C-terminus the first VH, the first CL, the first heavy chain CH1 hinge region, and the first Fc domain monomer; and/or the elements comprised in polypeptide chain VI-D comprise from N-terminus to C-terminus the first VL and the heavy chain CH1 region.

In certain embodiments, the adjacent elements comprised in the polypeptide chain VI-A are optionally connected to each other by a peptide linker or not; the adjacent elements comprised in the polypeptide chain VI-B are optionally connected to each other by a peptide linker or not; the adjacent elements comprised in the polypeptide chain VI-C are optionally connected to each other by a peptide linker or not; and/or the adjacent elements comprised in the polypeptide chain VI-D are optionally connected to each other by a peptide linker or not.

In certain embodiments, the peptide linkers are each independently the same peptide linker or a different peptide linker (e.g., a rigid peptide linker or a flexible peptide linker). In certain embodiments, each of the peptide linkers independently comprises 3 to 55 (e.g., 3-10, 10-25, 10-20, 10 to 17, 10 to 15, 10-25, 10-35, 10-45, 10-55) amino acid residues. In certain embodiments, the peptide linkers are each independently selected from peptide linkers comprising one or more glycine (G) and/or serine (S) residues. In certain embodiments, the peptide linkers each independently comprise 1, 2, 3, 4, 5, 6, 7, 8, 9 or 10 tandem copies of the peptide linker unit GGGGS (SEQ ID NO: 57), which may be represented by (GGGGS)ₙ, wherein n is selected from 1, 2, 3, 4, 5, 6, 7, 8, 9 or 10

(SEQ ID NOs: 57, 65, 55, 58, 66-68, 56, 69, or 70, respectively). In certain embodiments, the peptide linkers each independently comprise the amino acid sequence shown in any one of SEQ ID NOs: 55-58 or 65-70.

In certain embodiments, the first CL and the second CL comprises the amino acid sequence shown in SEQ ID NO: 53 or SEQ ID NO: 63, the heavy chain CH1 region comprises the amino acid sequence shown in SEQ ID NO: 54, and/or the first heavy chain CH1 hinge region comprises the amino acid sequence shown in SEQ ID NO: 62.

In certain embodiments, the first VH comprises the amino acid sequence shown in SEQ ID NO: 18 and the first VL comprises the amino acid sequence shown in SEQ ID NO: 17. In certain embodiments, the first VH comprises the amino acid sequence shown in SEQ ID NO: 60 and the first VL comprises the amino acid sequence shown in SEQ ID NO: 59. In certain embodiments, the second VH comprises the amino acid sequence shown in SEQ ID NO: 16 and the second VL comprises the amino acid sequence shown in SEQ ID NO: 15. In certain embodiments, the first VH comprises the amino acid sequence shown in SEQ ID NO: 18, the first VL comprises the amino acid sequence shown in SEQ ID NO: 17, the second VH comprises the amino acid sequence shown in SEQ ID NO: 16, and the second VL comprises the amino acid sequence shown in SEQ ID NO: 15. In certain embodiments, the first VH comprises the amino acid sequence shown in SEQ ID NO: 60, the first VL comprises the amino acid sequence shown in SEQ ID NO: 59, the second VH comprises the amino acid sequence shown in SEQ ID NO: 16, and the second VL comprises the amino acid sequence shown in SEQ ID NO: 15.

In certain embodiments, the first Fc domain monomer comprises the amino acid sequence shown in SEQ ID NO: 49 and the second Fc domain monomer comprises the amino acid sequence shown in SEQ ID NO: 50. In certain embodiments, the first Fc domain monomer comprises the amino acid sequence shown in SEQ ID NO: 51 and the second Fc domain monomer comprises the amino acid sequence shown in SEQ ID NO: 52.

In certain embodiments, the first Fc domain monomer comprises the amino acid sequence shown in SEQ ID NO: 50 and the second Fc domain monomer comprises the amino acid sequence shown in SEQ ID NO: 49. In certain embodiments, the first Fc domain monomer comprises the amino acid sequence shown in SEQ ID NO: 52 and the second Fc domain monomer comprises the amino acid sequence shown in SEQ ID NO: 51.

In certain embodiments, the bispecific antibody comprising a domain swap in the form of CrossMab^{VH-VL} comprises polypeptide chain V-A, polypeptide chain V-B, polypeptide chain V-C, and polypeptide chain V-D; wherein (a) the elements comprised in polypeptide chain V-A comprise a first VL and a first CL; the elements comprised in polypeptide chain V-B comprise a first VH, a first heavy chain CH1 region, and a first Fc domain monomer; the elements comprised in polypeptide chain V-C comprise a second VL, a second heavy chain CH1 hinge region, and a second Fc domain monomer; and the elements comprised in polypeptide chain V-D comprise a second VH and a second CL; or (b) the elements comprised in polypeptide chain V-A comprise a first VL and a first CL; the elements comprised in polypeptide chain V-B comprise a first VH, a first heavy chain CH1 region, and a second Fc domain monomer; the elements comprised in polypeptide chain V-C comprise a second VL, a second heavy chain CH1 hinge region, and a first Fc domain monomer; and the elements comprised in polypeptide chain V-D comprise a second VH and a second CL.

In certain embodiments, (a) the elements comprised in polypeptide chain V-A comprise from N-terminus to C-terminus the first VL and the first CL; the elements comprised in polypeptide chain V-B comprise from N-terminus to C-terminus the first VH, the first heavy chain CH1 region, and the first Fc domain monomer; the elements comprised in polypeptide chain V-C comprise from N-terminus to C-terminus the second VL, the second heavy chain CH1 hinge region, and the second Fc domain monomer, and/or the elements comprised in polypeptide chain V-D comprise from N-terminus to C-terminus the second VH and the second CL; or (b) the elements comprised in polypeptide chain V-A comprise from N-terminus to C-terminus the first VL and the first CL; the elements comprised in polypeptide chain V-B comprise from N-terminus to C-terminus the first VH, the first heavy chain CH1 region, and the second Fc domain monomer; the elements comprised in polypeptide chain V-C comprise from N-terminus to C-terminus the second VL, the second heavy chain CH1 hinge region, and the first Fc domain monomer, and/or the elements comprised in polypeptide chain V-D comprise from N-terminus to C-terminus the second VH and the second CL.

In certain embodiments, the adjacent elements comprised in the polypeptide chain V-A are optionally connected to each other by a peptide linker or not; the adjacent elements comprised in the polypeptide chain V-B are optionally connected to each other by a peptide linker or not; the adjacent elements comprised in the polypeptide chain V-C are optionally connected to each other by a peptide linker or not; and/or the adjacent elements comprised in the polypeptide chain V-D are optionally connected to each other by a peptide linker or not.

In certain embodiments, the peptide linkers are each independently the same peptide linker or a different peptide linker (e.g., a rigid peptide linker or a flexible peptide linker). In certain embodiments, each of the peptide linkers independently comprises 3 to 55 (e.g., 3-10, 10-25, 10-20, 10 to 17, 10 to 15, 10-25, 10-35, 10-45, 10-55) amino acid residues. In certain embodiments, the peptide linkers are each independently selected from peptide linkers comprising one or more glycine (G) and/or serine (S) residues. In certain embodiments, the peptide linkers each independently comprise 1, 2, 3, 4, 5, 6, 7, 8, 9 or 10 tandem copies of the peptide linker unit GGGGS (SEQ ID NO: 57), which may be represented by (GGGGS)ₙ, wherein n is selected from 1, 2, 3, 4, 5, 6, 7, 8, 9 or 10 (SEQ ID NOs: 57, 65, 55, 58, 66-68, 56, 69, or 70, respectively). In certain embodiments, the peptide linkers each independently comprise the amino acid sequence shown in any one of SEQ ID NOs: 55-58 or 65-70.

In certain embodiments, the first and second CL comprise the amino acid sequence shown in SEQ ID NO: 53 or SEQ ID NO: 63, the first and second heavy chain CH1 region comprise the amino acid sequence shown in SEQ ID NO: 54, and/or the second heavy chain CH1 hinge region comprises the amino acid sequence shown in SEQ ID NO: 62.

In certain embodiments, the first VH comprises the amino acid sequence shown in SEQ ID NO: 18 and the first VL comprises the amino acid sequence shown in SEQ ID NO: 17. In certain embodiments, the first VH comprises the amino acid sequence shown in SEQ ID NO: 60 and the first VL comprises the amino acid sequence shown in SEQ ID NO: 59. In certain embodiments, the second VH comprises the amino acid sequence shown in SEQ ID NO: 16 and the second VL comprises the amino acid sequence shown in SEQ ID NO: 15. In certain embodiments, the first VH comprises the amino acid sequence shown in SEQ ID NO: 18, the first VL comprises the amino acid sequence shown in SEQ ID NO: 17, the second VH comprises the amino acid sequence shown in SEQ ID NO: 16, and the second VL comprises the amino acid sequence shown in SEQ ID NO: 15. In certain embodiments, the first VH comprises the amino acid sequence shown in SEQ ID NO: 60, the first VL comprises the amino acid sequence shown in SEQ ID NO: 59, the second VH comprises the amino acid sequence shown in SEQ ID NO: 16, and the second VL comprises the amino acid sequence shown in SEQ ID NO: 15.

In certain embodiments, the first Fc domain monomer comprises the amino acid sequence shown in SEQ ID NO: 49 and the second Fc domain monomer comprises the amino acid sequence shown in SEQ ID NO: 50. In certain embodiments, the first Fc domain monomer comprises the amino acid sequence shown in SEQ ID NO: 51 and the second Fc domain monomer comprises the amino acid sequence shown in SEQ ID NO: 52.

In certain embodiments, the first Fc domain monomer comprises the amino acid sequence shown in SEQ ID NO: 50 and the second Fc domain monomer comprises the amino acid sequence shown in SEQ ID NO: 49. In certain embodiments, the first Fc domain monomer comprises the amino acid sequence shown in SEQ ID NO: 52 and the second Fc domain monomer comprises the amino acid sequence shown in SEQ ID NO: 51.

In certain embodiments, the bispecific antibody comprising a domain swap in the form of CrossMab^{Fab} comprises polypeptide chain V-A, polypeptide chain V-B, polypeptide chain V-C, and polypeptide chain V-D; wherein (a) the elements comprised in polypeptide chain V-A comprise a first VL and a first CL; the elements comprised in polypeptide chain V-B comprise a first VH, a first heavy chain CH1 region, and a first Fc domain monomer; the elements comprised in polypeptide chain V-C comprise a second VL, a second CL, a second heavy chain CH1 hinge region, and a second Fc domain monomer; and the elements comprised in polypeptide chain V-D comprise a second VH and second heavy chain CH1 region; or (b) the elements comprised in polypeptide chain V-A comprise a first VL and a first CL; the elements comprised in polypeptide chain V-B comprise a first VH, a first heavy chain CH1 region, and a second Fc domain monomer; the elements comprised in polypeptide chain V-C comprise a second VL, a second CL, a second heavy chain CH1 hinge region, and a first Fc domain monomer; and the elements comprised in polypeptide chain V-D comprise a second VH and a second heavy chain CH1 region.

In certain embodiments, (a) the elements comprised in polypeptide chain V-A comprise from N-terminus to C-terminus the first VL and the first CL; the elements comprised in polypeptide chain V-B comprise from N-terminus to C-terminus the first VH, the first heavy chain CH1 region, and the first Fc domain monomer; the elements comprised in polypeptide chain V-C comprise from N-terminus to C-terminus the second VL, the second CL, the second heavy chain CH1 hinge region, and the second Fc domain monomer, and/or the elements comprised in polypeptide chain V-D comprise from N-terminus to C-terminus the second VH and the second heavy chain CH1 region; or (b) the elements comprised in polypeptide chain V-A comprise from N-terminus to C-terminus the first VL and the first CL; the elements comprised in polypeptide chain V-B comprise from N-terminus to C-terminus the first VH, the first heavy chain CH1 region, and the second Fc domain monomer; the elements comprised in polypeptide chain V-C comprise from N-terminus to C-terminus the second VL, the second CL, the second heavy chain CH1 hinge region, and the first Fc domain monomer, and/or the elements comprised in polypeptide chain V-D comprise from N-terminus to C-terminus the second VH and the second heavy chain CH1 region.

In certain embodiments, the adjacent elements comprised in the polypeptide chain V-A are optionally connected to each other by a peptide linker or not; the adjacent elements comprised in the polypeptide chain V-B are optionally connected to each other by a peptide linker or not; the adjacent elements comprised in the polypeptide chain V-C are optionally connected to each other by a peptide linker or not; and/or the adjacent elements comprised in the polypeptide chain V-D are optionally connected to each other by a peptide linker or not.

In certain embodiments, the peptide linkers are each independently the same peptide linker or a different peptide linker (e.g., a rigid peptide linker or a flexible peptide linker). In certain embodiments, each of the peptide linkers independently comprises 3 to 55 (e.g., 3-10, 10-25, 10-20, 10 to 17, 10 to 15, 10-25, 10-35, 10-45, 10-55) amino acid residues. In certain embodiments, the peptide linkers are each independently selected from peptide linkers comprising one or more glycine (G) and/or serine (S) residues. In certain embodiments, the peptide linkers each independently comprise 1, 2, 3, 4, 5, 6, 7, 8, 9 or 10 tandem copies of the peptide linker unit GGGGS (SEQ ID NO: 57), which may be represented by (GGGGS)ₙ, wherein n is selected from 1, 2, 3, 4, 5, 6, 7, 8, 9 or 10 (SEQ ID NOs: 57, 65, 55, 58, 66-68, 56, 69, or 70, respectively). In certain embodiments, the peptide linkers each independently comprise the amino acid sequence shown in any one of SEQ ID NOs: 55-58 or 65-70.

In certain embodiments, the first and second CL comprise the amino acid sequence shown in SEQ ID NO: 53 or SEQ ID NO: 63, the first and second heavy chain CH1 region comprise the amino acid sequence shown in SEQ ID NO: 54, and/or the second heavy chain CH1 hinge region comprises the amino acid sequence shown in SEQ ID NO: 62.

In certain embodiments, the first VH comprises the amino acid sequence shown in SEQ ID NO: 18 and the first VL comprises the amino acid sequence shown in SEQ ID NO: 17. In certain embodiments, the first VH comprises the amino acid sequence shown in SEQ ID NO: 60 and the first VL comprises the amino acid sequence shown in SEQ ID NO: 59. In certain embodiments, the second VH comprises the amino acid sequence shown in SEQ ID NO: 16 and the second VL comprises the amino acid sequence shown in SEQ ID NO: 15. In certain embodiments, the first VH comprises the amino acid sequence shown in SEQ ID NO: 18, the first VL comprises the amino acid sequence shown in SEQ ID NO: 17, the second VH comprises the amino acid sequence shown in SEQ ID NO: 16, and the second VL comprises the amino acid sequence shown in SEQ ID NO: 15. In certain embodiments, the first VH comprises the amino acid sequence shown in SEQ ID NO: 60, the first VL comprises the amino acid sequence shown in SEQ ID NO: 59, the second VH comprises the amino acid sequence shown in SEQ ID NO: 16, and the second VL comprises the amino acid sequence shown in SEQ ID NO: 15.

In certain embodiments, the first Fc domain monomer comprises the amino acid sequence shown in SEQ ID NO: 49 and the second Fc domain monomer comprises the amino acid sequence shown in SEQ ID NO: 50. In certain embodiments, the first Fc domain monomer comprises the amino acid sequence shown in SEQ ID NO: 51 and the second Fc domain monomer comprises the amino acid sequence shown in SEQ ID NO: 52.

In certain embodiments, the first Fc domain monomer comprises the amino acid sequence shown in SEQ ID NO: 50 and the second Fc domain monomer comprises the amino acid sequence shown in SEQ ID NO: 49. In certain embodiments, the first Fc domain monomer comprises the amino acid sequence shown in SEQ ID NO: 52 and the second Fc domain monomer comprises the amino acid sequence shown in SEQ ID NO: 51.

scFv-scFv Structures

In certain embodiments, the first antigen-binding domain and the second antigen-binding domain are both scFvs.

In certain embodiments, the bispecific antibody comprises polypeptide chain VII-A and polypeptide chain VII-B; wherein (a) the elements comprised in polypeptide chain VII-A comprise a first VL, a first VH, a first heavy chain CH1 hinge region, and a first Fc domain monomer; and the elements comprised in polypeptide chain VII-B comprise a second VL, a second VH, a second heavy chain CH1 hinge region, and a second Fc domain monomer; or (b) the elements comprised in polypeptide chain VII-A comprise a first VL, a first VH, a first heavy chain CH1 hinge region and a second Fc domain monomer; and the elements comprised in polypeptide chain VII-B comprise a second VL, a second VH, a second heavy chain CH1 hinge region, and a first Fc domain monomer.

In certain embodiments, (a) the elements comprised in polypeptide chain VII-A comprise from N-terminus to C-terminus (i) the first VL, the first VH, the first heavy chain CH1 hinge region, and the first Fc domain monomer, or (ii) the first VH, the first VL, the first heavy chain CH1 hinge region, and the first Fc domain monomer; and/or the elements comprised in polypeptide chain VII-B comprise from N-terminus to C-terminus (i) the second VL, the second VH, the second heavy chain CH1 hinge region, and the second Fc domain monomer, or (ii) the second VH, the second VL, the second heavy chain CH1 hinge region, and the second Fc domain monomer; or (b) the elements comprised in polypeptide chain VII-A comprise from N-terminus to C-terminus (i) the first VL, the first VH, the first heavy chain CH1 hinge region, and the second Fc domain monomer, or (ii) the first VH, the first VL, the first heavy chain CH1 hinge region, and the second Fc domain monomer; and/or the elements comprised in polypeptide chain VII-B comprise from N-terminus to C-terminus (i) the second VL, the second VH, the second heavy chain CH1 hinge region, and the first Fc domain monomer, or (ii) the second VH, the second VL, the second heavy chain CH1 hinge region, and the first Fc domain monomer.

In certain embodiments, the adjacent elements comprised in the polypeptide chain VII-A are optionally connected to each other by a peptide linker or not and/or the adjacent elements comprised in the polypeptide chain VII-B are optionally connected to each other by a peptide linker or not.

In certain embodiments, the peptide linkers are each independently the same peptide linker or a different peptide linker (e.g., a rigid peptide linker or a flexible peptide linker). In certain embodiments, each of the peptide linkers independently comprises 3 to 55 (e.g., 3-10, 10-25, 10-20, 10 to 17, 10 to 15, 10-25, 10-35, 10-45, 10-55) amino acid residues. In certain embodiments, the peptide linkers are each independently selected from peptide linkers comprising one or more glycine (G) and/or serine (S) residues. In certain embodiments, the peptide linkers each independently comprise 1, 2, 3, 4, 5, 6, 7, 8, 9 or 10 tandem copies of the peptide linker unit GGGGS (SEQ ID NO: 57), which may be represented by (GGGGS)ₙ, wherein n is selected from 1, 2, 3, 4, 5, 6, 7, 8, 9 or 10 (SEQ ID NOs: 57, 65, 55, 58, 66-68, 56, 69, or 70, respectively). In certain embodiments, the peptide linkers each independently comprise the amino acid sequence shown in any one of SEQ ID NOs: 55-58 or 65-70.

In certain embodiments, the first and second heavy chain CH1 hinge region comprises the amino acid sequence shown in SEQ ID NO: 62.

In certain embodiments, the first VH comprises the amino acid sequence shown in SEQ ID NO: 18 and the first VL comprises the amino acid sequence shown in SEQ ID NO: 17. In certain embodiments, the first VH comprises the amino acid sequence shown in SEQ ID NO: 60 and the first VL comprises the amino acid sequence shown in SEQ ID NO: 59. In certain embodiments, the second VH comprises the amino acid sequence shown in SEQ ID NO: 16 and the second VL comprises the amino acid sequence shown in SEQ ID NO: 15. In certain embodiments, the first VH comprises the amino acid sequence shown in SEQ ID NO: 18, the first VL comprises the amino acid sequence shown in SEQ ID NO: 17, the second VH comprises the amino acid sequence shown in SEQ ID NO: 16, and the second VL comprises the amino acid sequence shown in SEQ ID NO: 15. In certain embodiments, the first VH comprises the amino acid sequence shown in SEQ ID NO: 60, the first VL comprises the amino acid sequence shown in SEQ ID NO: 59, the second VH comprises the amino acid sequence shown in SEQ ID NO: 16, and the second VL comprises the amino acid sequence shown in SEQ ID NO: 15.

In certain embodiments, the first Fc domain monomer comprises the amino acid sequence shown in SEQ ID NO: 49 and the second Fc domain monomer comprises the amino acid sequence shown in SEQ ID NO: 50. In certain embodiments, the first Fc domain monomer comprises the amino acid sequence shown in SEQ ID NO: 51 and the second Fc domain monomer comprises the amino acid sequence shown in SEQ ID NO: 52.

In certain embodiments, the first Fc domain monomer comprises the amino acid sequence shown in SEQ ID NO: 50 and the second Fc domain monomer comprises the amino acid sequence shown in SEQ ID NO: 49. In certain embodiments, the first Fc domain monomer comprises the amino acid sequence shown in SEQ ID NO: 52 and the second Fc domain monomer comprises the amino acid sequence shown in SEQ ID NO: 51.

In certain embodiments, the polypeptide chain VII-A comprises the amino acid sequence shown in SEQ ID NO: 14, and/or the polypeptide chain VII-B comprises the amino acid sequence shown in SEQ ID NO: 8.

In certain exemplary embodiments, a bispecific antibody of an ADC provided herein comprises:
a polypeptide chain I-A comprising the amino acid sequence shown in SEQ ID NO: 1, a polypeptide chain I-B comprising the amino acid sequence shown in SEQ ID NO: 2, and a polypeptide chain I-C comprising the amino acid sequence shown in SEQ ID NO: 3;
a polypeptide chain I-A comprising the amino acid sequence shown in SEQ ID NO: 1, a polypeptide chain I-B comprising the amino acid sequence shown in SEQ ID NO: 9, and a polypeptide chain I-C comprising the amino acid sequence shown in SEQ ID NO: 10;
a polypeptide chain II-A comprising the amino acid sequence shown in SEQ ID NO: 4, a polypeptide chain II-B comprising the amino acid sequence shown in SEQ ID NO: 5, and a polypeptide chain II-C comprising the amino acid sequence shown in SEQ ID NO: 6;
a polypeptide chain II-A comprising the amino acid sequence shown in SEQ ID NO: 4, a polypeptide chain II-B comprising the amino acid sequence shown in SEQ ID NO: 7, and a polypeptide chain II-C comprising the amino acid sequence shown in SEQ ID NO: 8;
a polypeptide chain IV-A comprising the amino acid sequence shown in SEQ ID NO: 4, a polypeptide chain IV-B comprising the amino acid sequence shown in SEQ ID NO: 7, and a polypeptide chain IV-C comprising the amino acid sequence shown in SEQ ID NO: 11;
a polypeptide chain V-A comprising the amino acid sequence shown in SEQ ID NO: 1, a polypeptide chain V-B comprising the amino acid sequence shown in SEQ ID NO: 9, a polypeptide chain V-C comprising the amino acid sequence shown in SEQ ID NO: 13, and a polypeptide chain V-D comprising the amino acid sequence shown in SEQ ID NO: 12; or
a polypeptide chain VII-A comprising the amino acid sequence shown in SEQ ID NO: 14 and a polypeptide chain VII-B comprising the amino acid sequence shown in SEQ ID NO: 8.

In certain embodiments, the ADCs comprising the bispecific antibodies have an enhanced tumor suppressive effect relative to a monospecific anti-c-MET antibody and/or a monospecific anti-EGFR antibody; wherein the CDR amino acid sequences of CDRs of the monospecific anti-c-MET are identical to the amino acid sequences of CDRs sequence of the first antigen-binding domain, and the amino acid sequences of CDRs sequence of the monospecific anti-EGFR antibody are identical to the CDR amino acid sequences of CDRs of the second antigen-binding domain.

In certain embodiments, the tumor suppressive effect includes inhibition of EGFR and c-MET signaling, antibody-dependent cell-mediated cytotoxicity (ADCC) activity, and/or complement-dependent cytotoxicity (CDC) activity.

### Drug-Linkers of the ADCs

The ADCs provided herein comprise a bispecific antibody as described herein and further comprise a cytotoxic drug fragment D, which is linked to the antibody via a linker -M-L-E-.

In some embodiments, the linkers described in European patent publication, EP 4349372 are hereby incorporated by reference. In some embodiments, the linkers (M-L-E) and the individual components M, L and E as described in European patent publication, EP 4349372, are hereby incorporated by reference. In some embodiments, the cytotoxic drugs described in European patent publication, EP 4349372 are hereby incorporated by reference. In some embodiments, the linkers/cytotoxic drugs described in European patent publication, EP 4349372 are hereby incorporated by reference.

In some embodiments, M in the ADC is

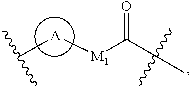

wherein ring A is a 5-6 membered aliphatic heterocyclic ring or a 5-20 membered aromatic ring system, and the aliphatic heterocyclic and aromatic ring systems are optionally substituted by one or more groups independently selected from the group consisting of oxo (═O), halogen, cyano, amino, carboxyl, thiol and C₁₋₆ alkyl group substitution; and M1 is selected from single bond and C₁₋₂₀ alkylene group, C₂-20 alkenylene group, C₂₋₂₀ alkynylene group, or amine group.

In some embodiments, M is

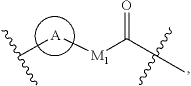

wherein ring A is a 5-membered aliphatic heterocyclic ring, a 6-membered heteroaromatic ring, or a polycyclic ring formed by connecting more than one 6-membered aromatic heterocyclic ring and a benzene ring through a single bond or by connecting more than one 6-membered heteroaromatic rings through single bonds, and the aliphatic heterocyclic ring is optionally substituted by one or more selected from the group consisting of oxo (═O), halogen and C₁₋₄ alkyl group substitution; and M1 is selected from single bond, C₁₋₂₀ alkylene, C₂₋₂₀ alkenylene, C₂₋₂₀ alkynylene, or amine group.

In some embodiments, M is

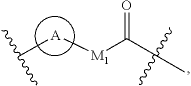

wherein ring A is selected from

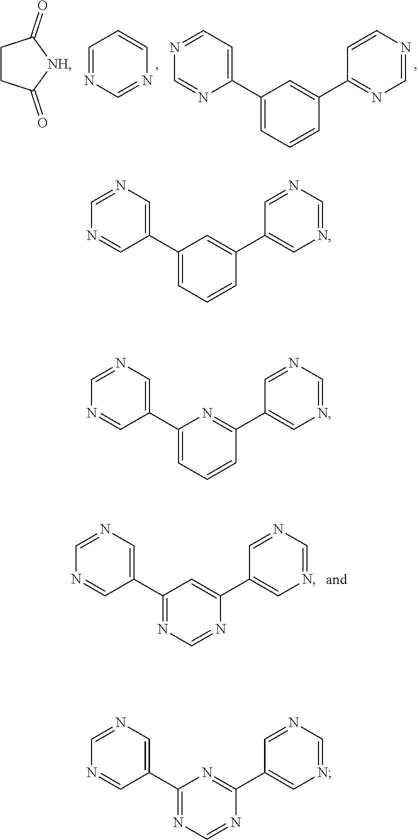

and M₁ is selected from single bond and C₁₋₆ alkylene, C₂₋₆ alkenylene, C₂₋₆ alkynylene or amine group.

In some embodiments, M is selected from

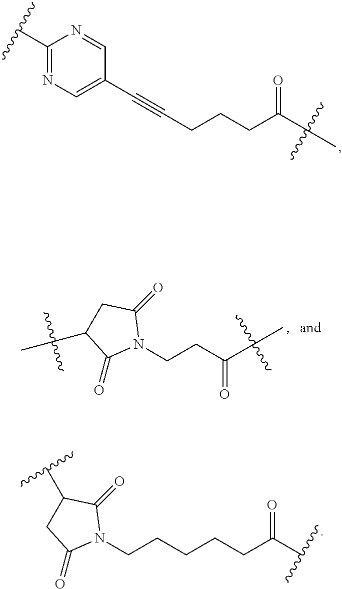 [SMILES: *C(=O)CCCC#Cc1cnc(*)nc1 ; *C(=O)CCN1C(=O)CC(*)C1=O ; "," ; "and" ; *C(=O)CCCCCN1C(=O)CC(*)C1=O]

In some embodiments, M is

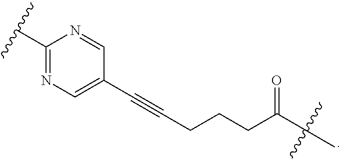 [SMILES: *C(=O)CCCC#Cc1cnc(*)nc1]

In some embodiments, M is selected from

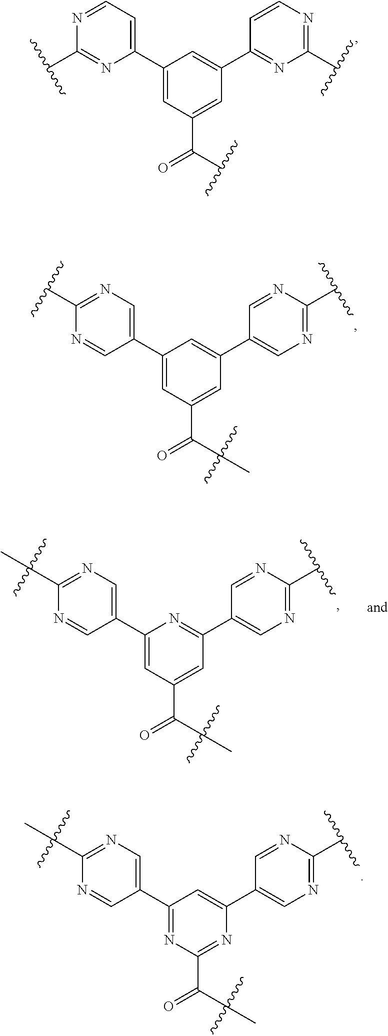 [SMILES: *C(=O)c1cc(-c2ccnc(*)n2)cc(-c2ccnc(*)n2)c1 ; *C(=O)c1cc(-c2cnc(*)nc2)cc(-c2cnc(*)nc2)c1 ; *C(=O)c1cc(-c2cnc(*)nc2)nc(-c2cnc(*)nc2)c1 ; *C(=O)c1nc(-c2cnc(*)nc2)cc(-c2cnc(*)nc2)n1]

In some embodiments, M is selected from

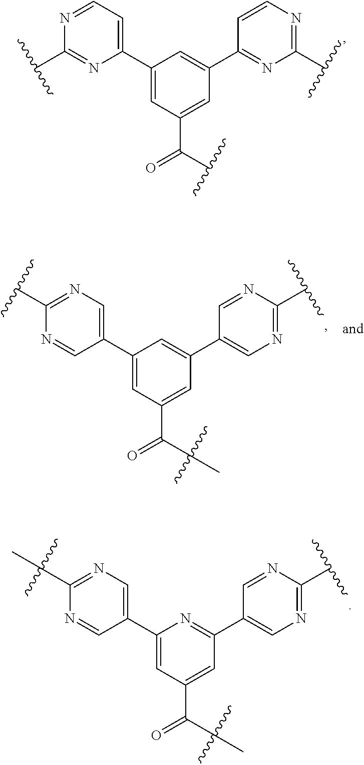 [SMILES: *C(=O)c1cc(-c2ccnc(*)n2)cc(-c2ccnc(*)n2)c1 ; *C(=O)c1cc(-c2cnc(*)nc2)cc(-c2cnc(*)nc2)c1 ; *C(=O)c1cc(-c2cnc(*)nc2)nc(-c2cnc(*)nc2)c1]

In some embodiments, L in the ADC is selected from a structure consisting of one or more of the following: C₁₋₆ alkylene, —N(R′)—, carbonyl, —O—, natural amino acids or unnatural amino acids and their analogs (such as Ala, Arg, Asn, Asp, Cit, Cys, Gln, Glu, Gly, His, Ile, Leu, Lys, Met, Phe, Pro, Ser, Thr, Trp, Tyr, Val, Lys(COCH₂CH₂(OCH₂CH₂)_{S}OCH₃)), and short peptides composed of amino acids (such as Ala-Ala, Ala-Lys, Ala-Lys(Ac), Ala-Pro, Gly-Glu, Gly-Gly, Phe-Lys, Phe-Lys(Ac), Val-Ala, Val-Lys, Val-Lys(Ac), Val-Cit, Ala-Ala-Ala, Ala-Ala-Asn, Leu-Ala-Glu, Gly-Gly-Arg, Gly-Glu-Gly, Gly-Gly-Gly, Gly-Ser-Lys, Glu-Val-Ala, Glu-Val-Cit, Ser-Ala-Pro, Val-Leu-Lys, Val-Lys-Ala, Val-Lys-Gly, Gly-Gly-Phe-Gly (GGFG (SEQ ID NO: 71)), Gly-Gly-Val-Ala (GGVA (SEQ ID NO: 72), Gly-Phe-Leu-Gly (GFLG (SEQ ID NO: 73)), Glu-Ala-Ala-Ala (EAAA (SEQ ID NO: 74)), and Gly-Gly-Gly-Gly-Gly (GGGGG (SEQ ID NO: 75))),

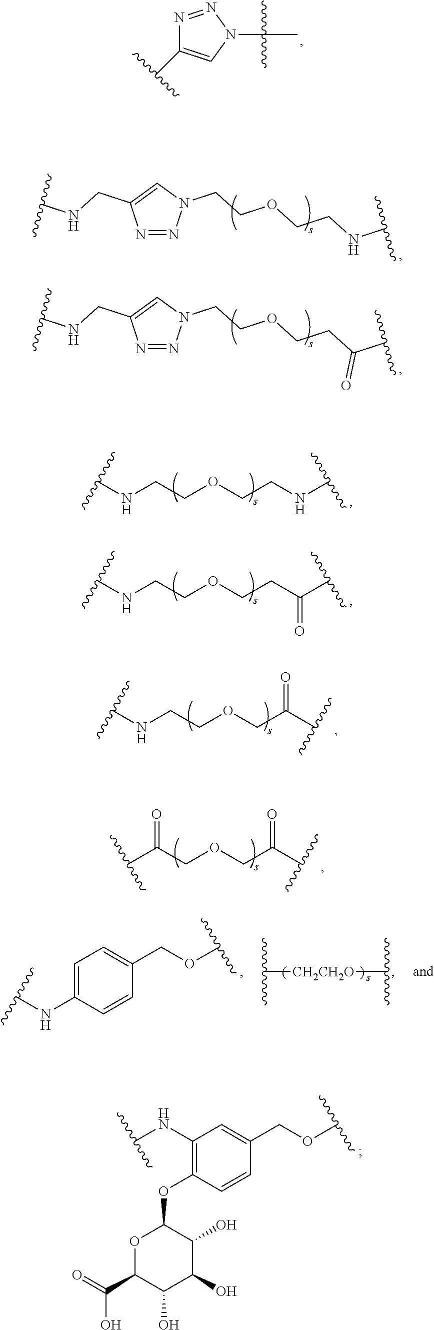

wherein R′ represents hydrogen, C₁₋₆ alkyl, or a polyethylene glycol fragment containing 1-10 ethylene oxide (EO) units; and s is selected from an integer of 1-20.

In some embodiments, L is selected from a structure consisting of one or more of the following: C₁₋₆ Alkylene, carbonyl, —NH—, Ala-Ala, Ala-Lys, Ala-Pro, Gly-Glu, Gly-Gly, Phe-Lys, Val-Ala, Val-Lys, Val-Cit, Ala-Ala-Ala, Ala-Ala-Asn, Leu-Ala-Glu, Gly-Gly-Arg, Gly-Glu-Gly, Gly-Gly-Gly, Gly-Ser-Lys, Glu-Val-Ala, Glu-Val-Cit, Ser-Ala-Pro, Val-Leu-Lys, Val-Lys-Ala, Val-Lys-Gly, Gly-Gly-Phe-Gly (GGFG (SEQ ID NO: 71)), Gly-Gly-Val-Ala (GGVA (SEQ ID NO: 72), Gly-Phe-Leu-Gly (GFLG (SEQ ID NO: 73)), Glu-Ala-Ala-Ala (EAAA (SEQ ID NO: 74)), and Gly-Gly-Gly-Gly-Gly (GGGGG (SEQ ID NO: 75)),

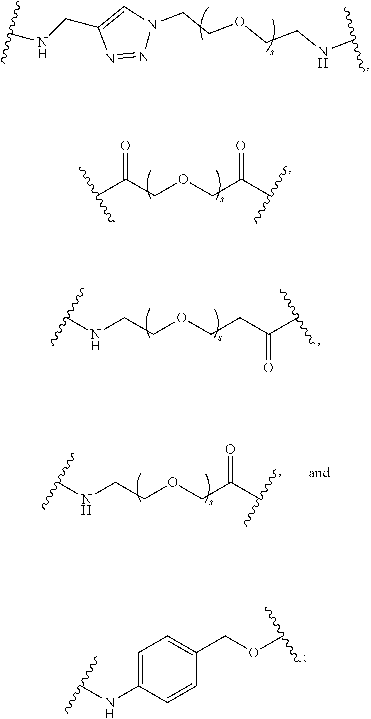

wherein s is selected from an integer from 1 to 20.

In some embodiments, L is selected from a structure consisting of one or more of the following:

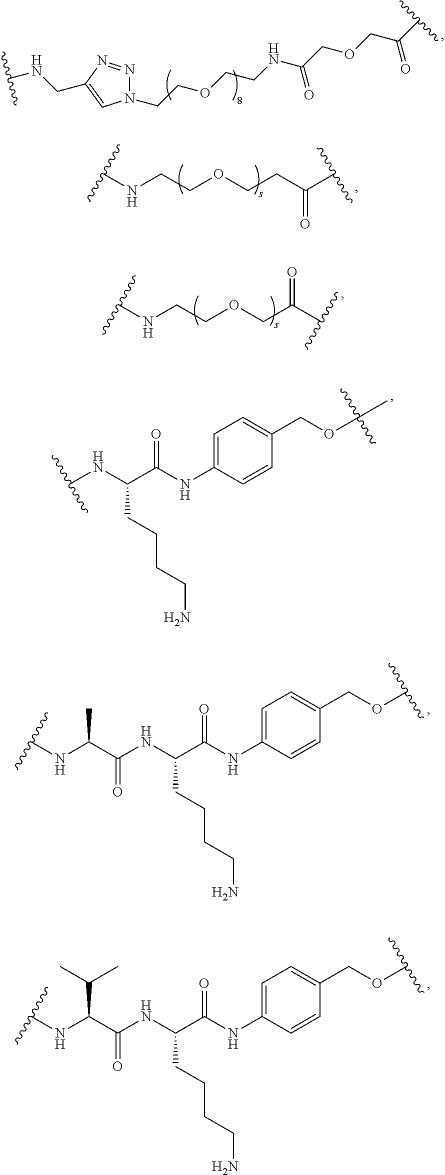

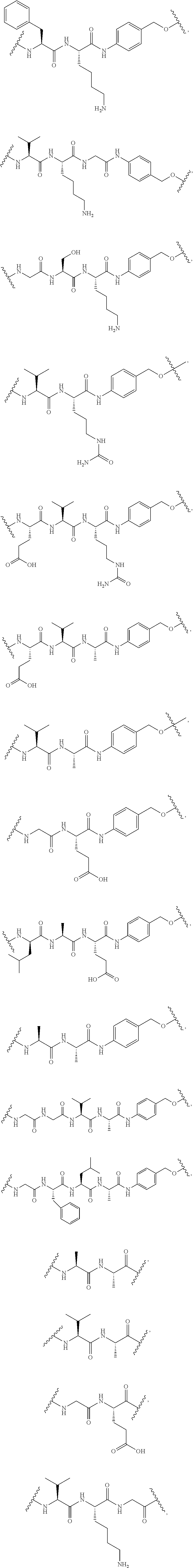

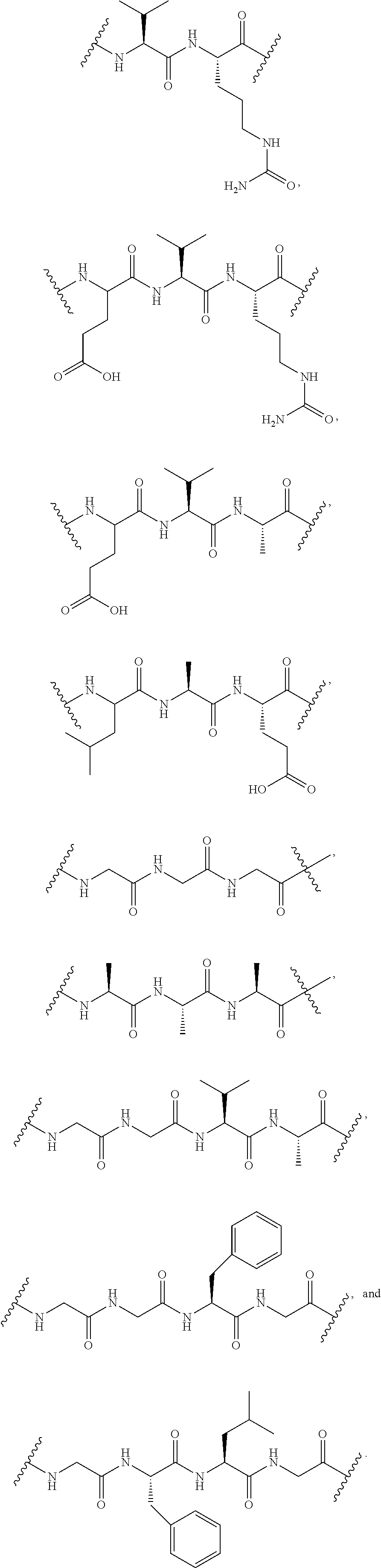

In some embodiments, L is selected from the following structures:

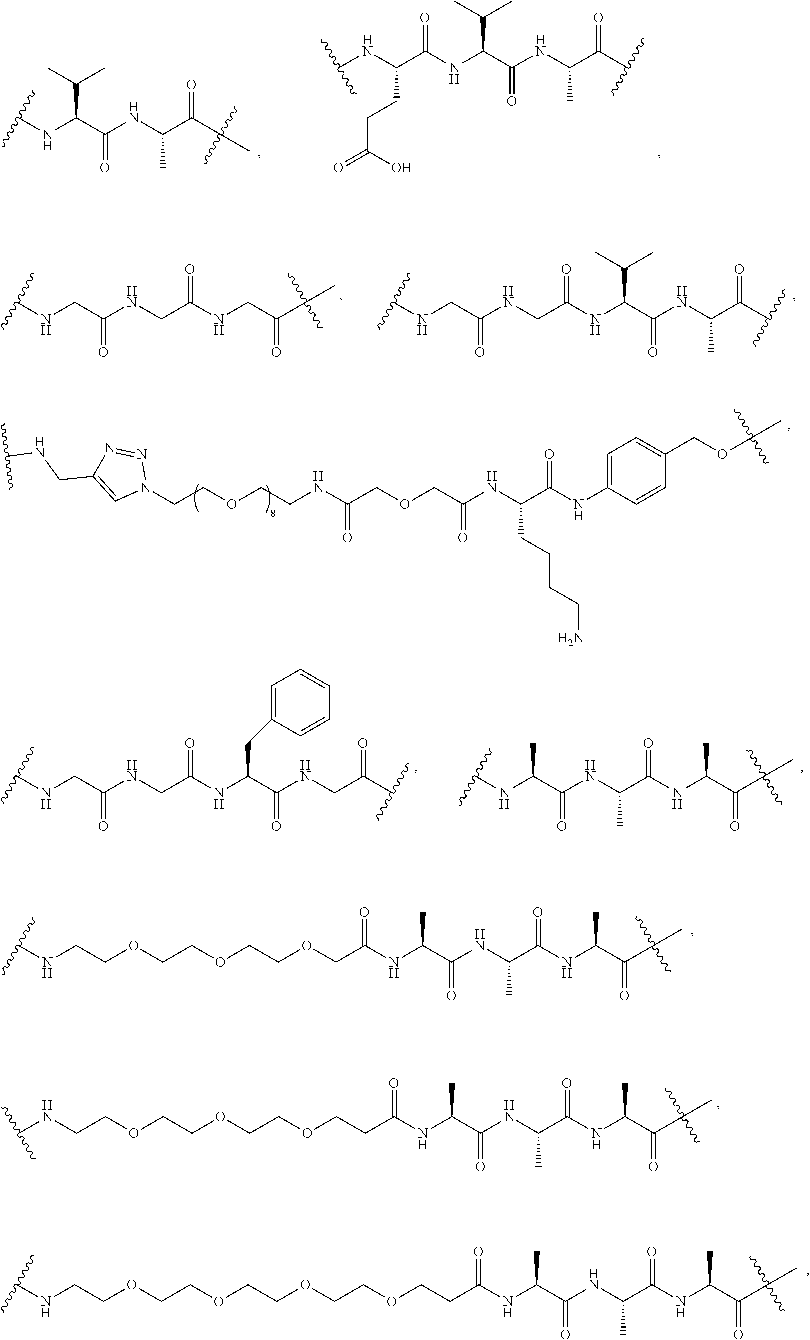

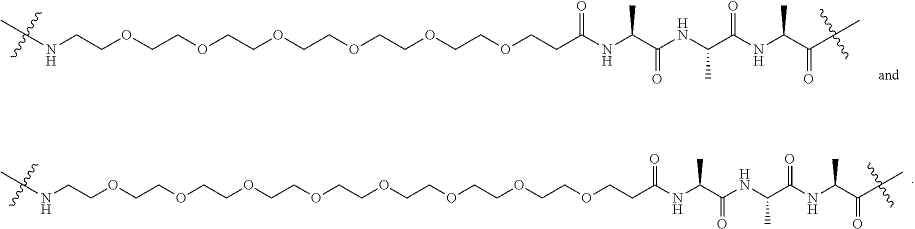 [SMILES: *NCCOCCOCCOCCOCCOCCOCCC(=O)N[C@@H](C)C(=O)N[C@@H](C)C(=O)N[C@@H](C)C(*)=O ; *NCCOCCOCCOCCOCCOCCOCCOCCOCCC(=O)N[C@@H](C)C(=O)N[C@@H](C)C(=O)N[C@@H](C)C(*)=O]

In some embodiments, L is selected from the following structures:

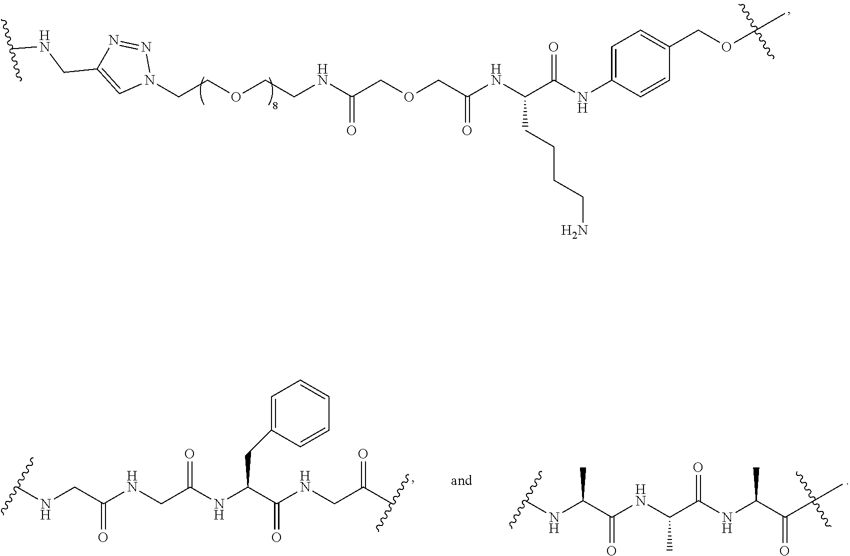

In some embodiments, L is selected from the following structures:

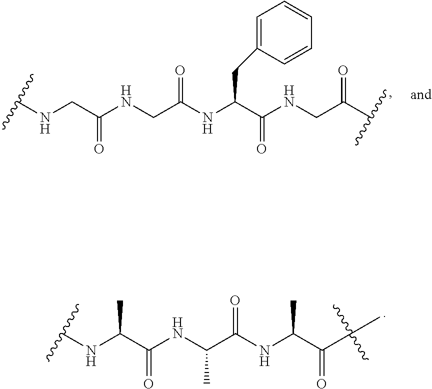 [SMILES: *NCC(=O)NCC(=O)N[C@@H](Cc1ccccc1)C(=O)NCC(*)=O ; *N[C@@H](C)C(=O)N[C@@H](C)C(=O)N[C@@H](C)C(*)=O]

In some embodiments, L is selected from the following structures:

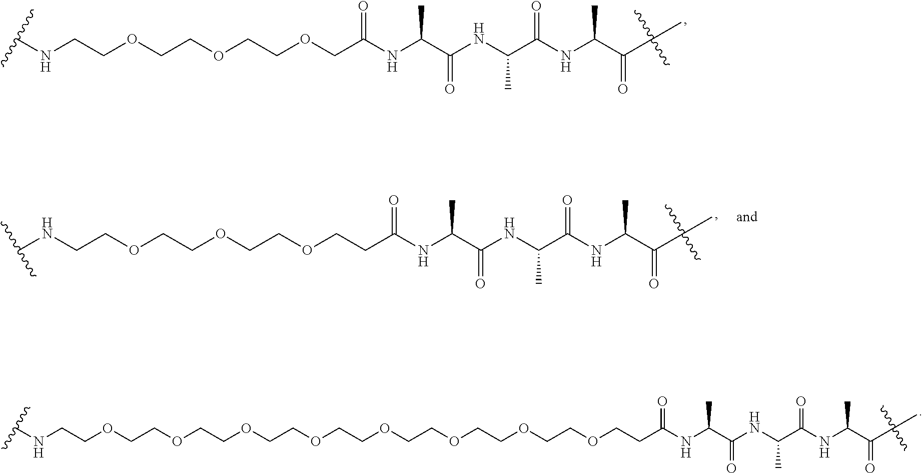 [SMILES: *NCCOCCOCCOCC(=O)N[C@@H](C)C(=O)N[C@@H](C)C(=O)N[C@@H](C)C(*)=O ; *NCCOCCOCCOCCC(=O)N[C@@H](C)C(=O)N[C@@H](C)C(=O)N[C@@H](C)C(*)=O ; *NCCOCCOCCOCCOCCOCCOCCOCCOCCC(=O)N[C@@H](C)C(=O)N[C@@H](C)C(=O)N[C@@H](C)C(*)=O]

In some embodiments, E in the ADC is a single bond or selected from the following structures:

### —NHCH₂—,

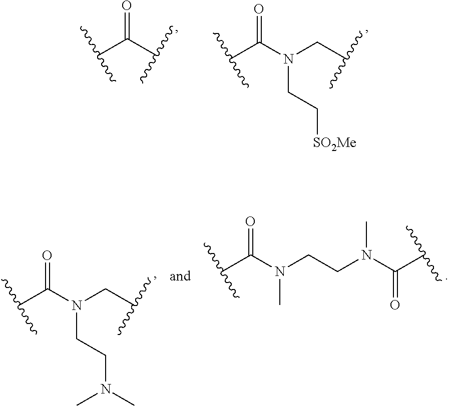

In some embodiments, E is a single bond, —NHCH₂—,

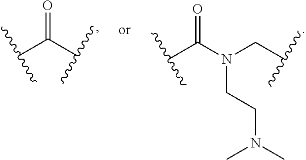

In some embodiments, E is —NHCH₂— or

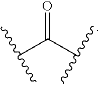 [SMILES: *C(*)=O]

In some embodiments, E is —NHCH₂—.

In some embodiments, E is a single bond.

In some embodiments, E is

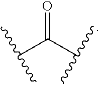 [SMILES: *C(*)=O]

In some embodiments, M is selected from

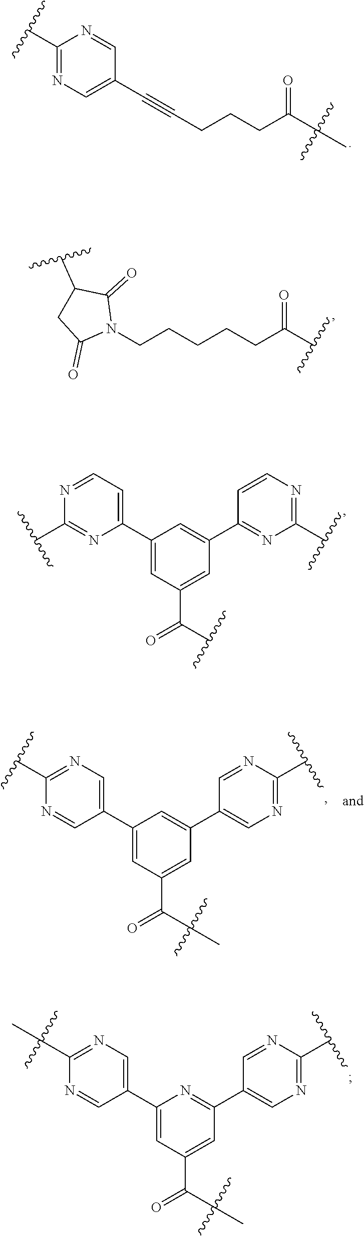 [SMILES: *C(=O)CCCC#Cc1cnc(*)nc1 ; *C(=O)CCCCCN1C(=O)CC(*)C1=O ; *C(=O)c1cc(-c2ccnc(*)n2)cc(-c2ccnc(*)n2)c1 ; *C(=O)c1cc(-c2cnc(*)nc2)cc(-c2cnc(*)nc2)c1 ; *C(=O)c1cc(-c2cnc(*)nc2)nc(-c2cnc(*)nc2)c1]

L is selected from

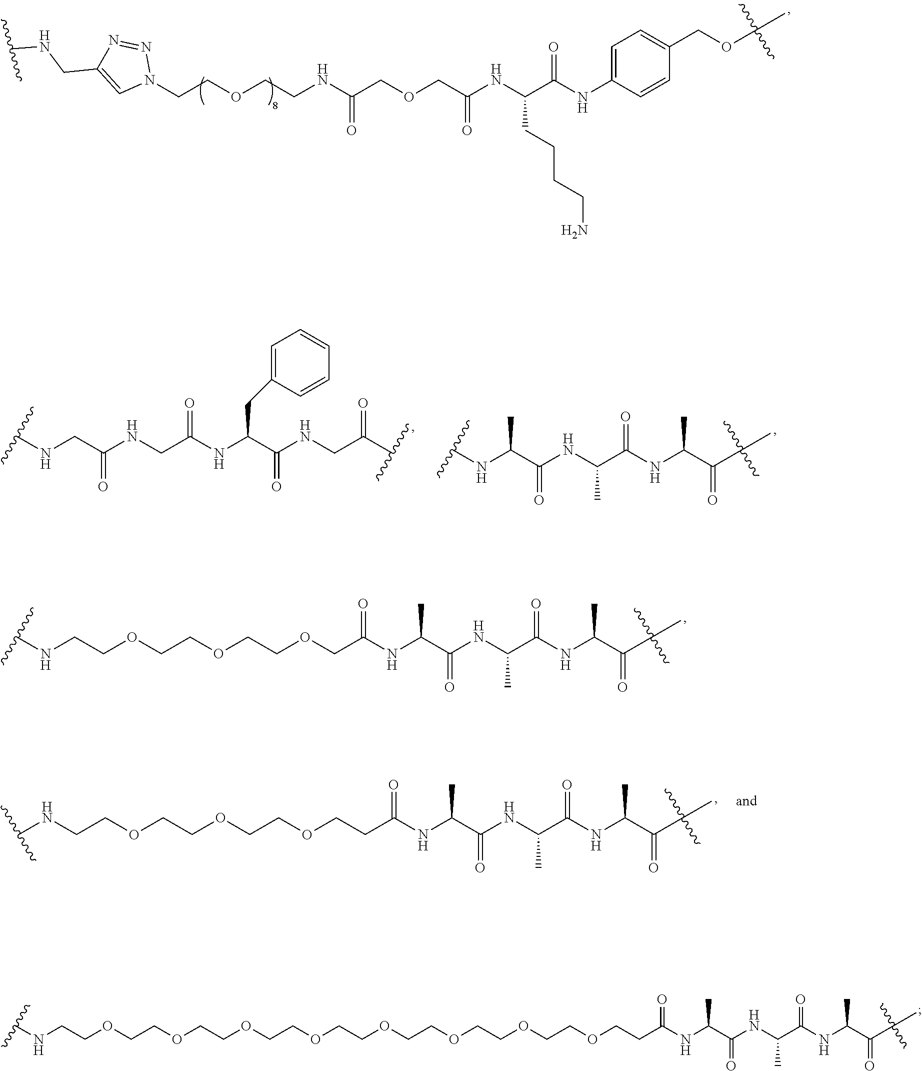

and E is selected from —NHCH₂— and

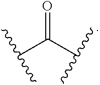 [SMILES: *C(*)=O]

In some embodiments of the ADCs provided herein,

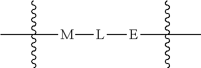

is selected from the following structures:

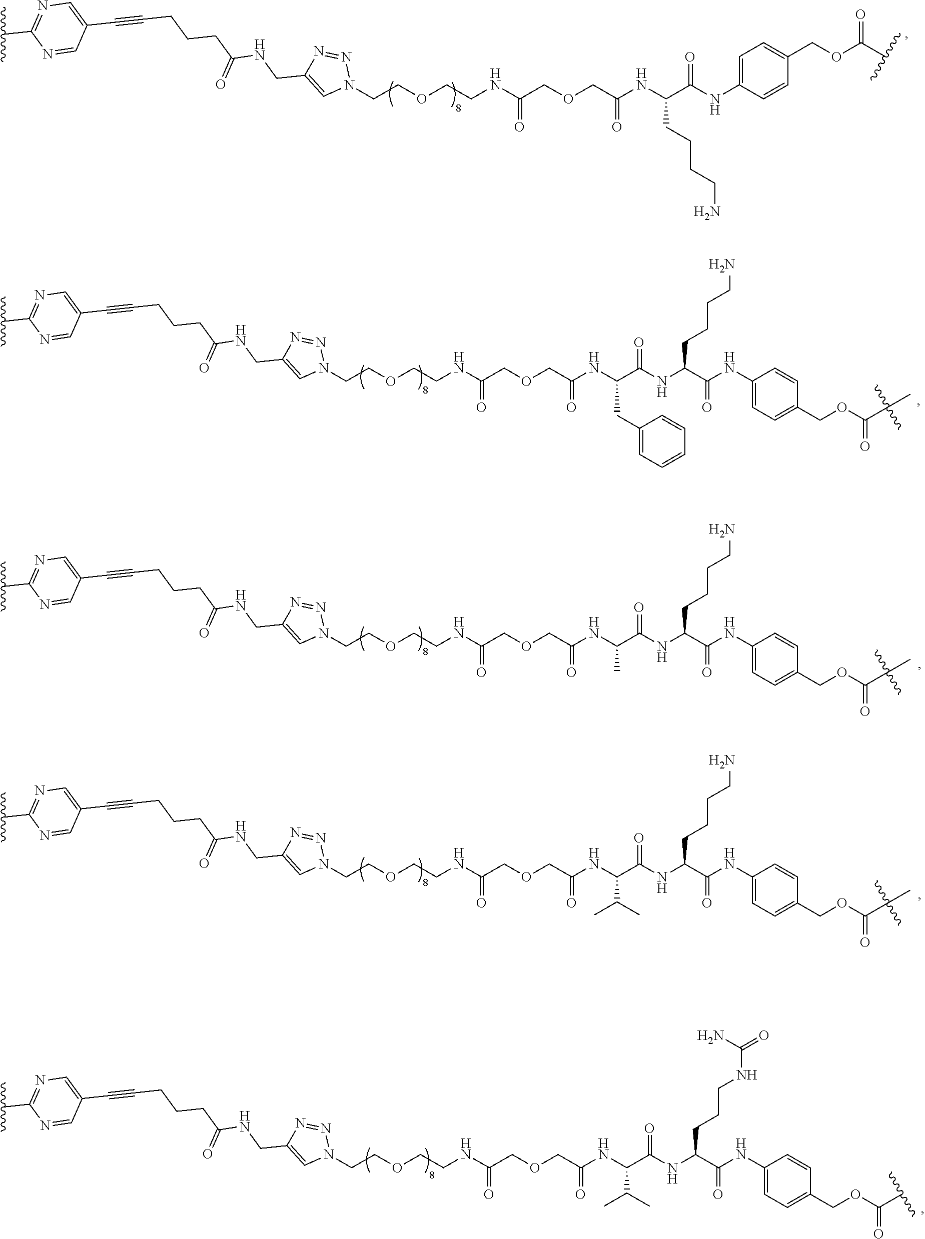

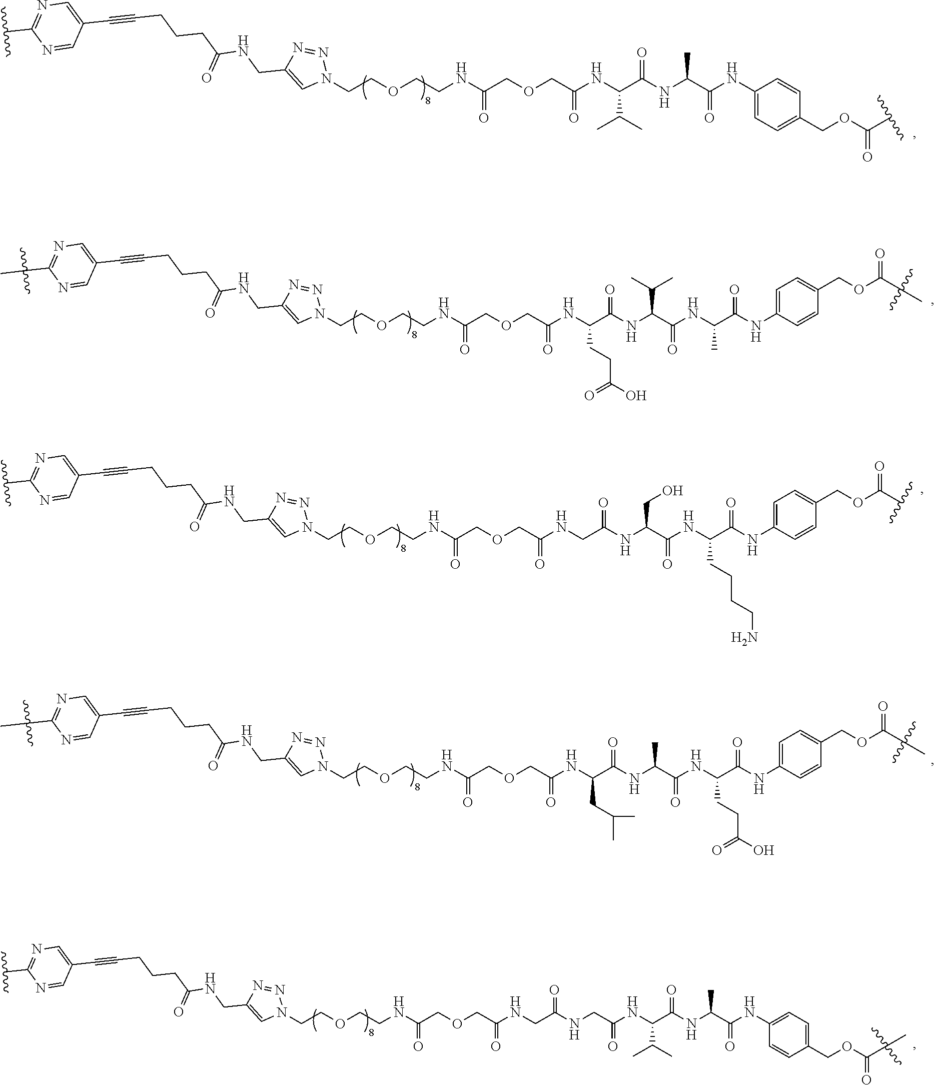

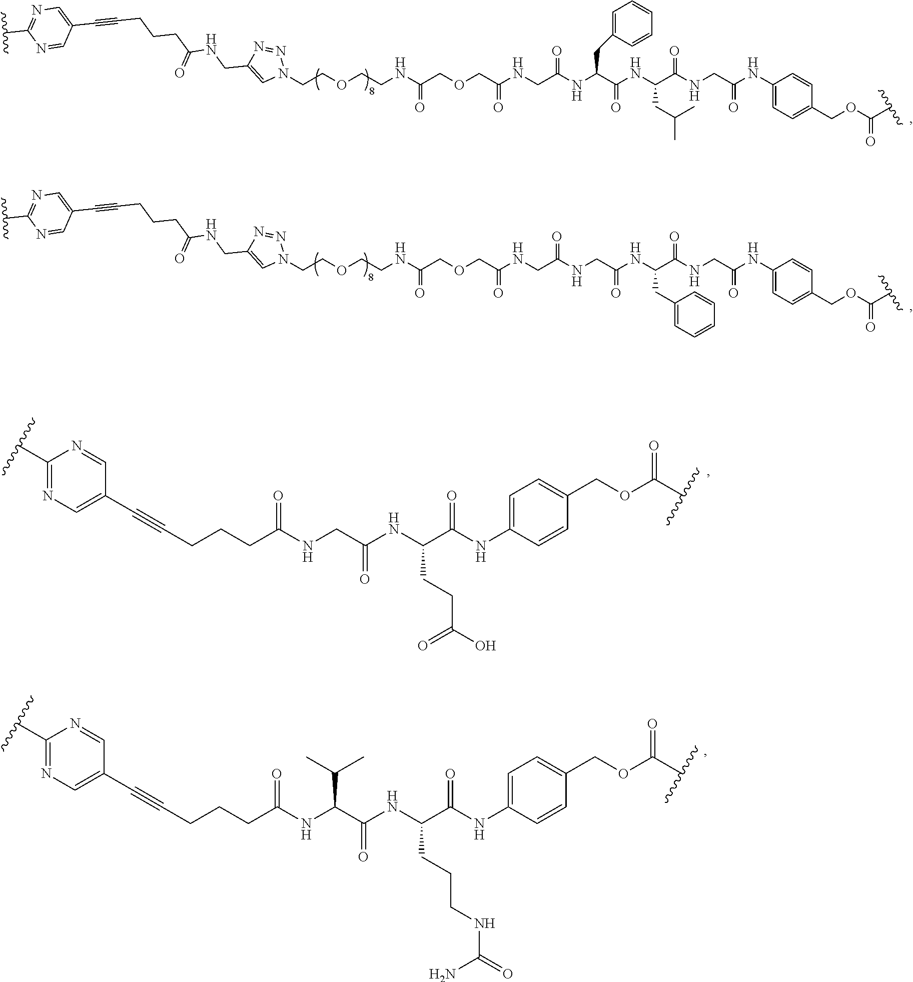

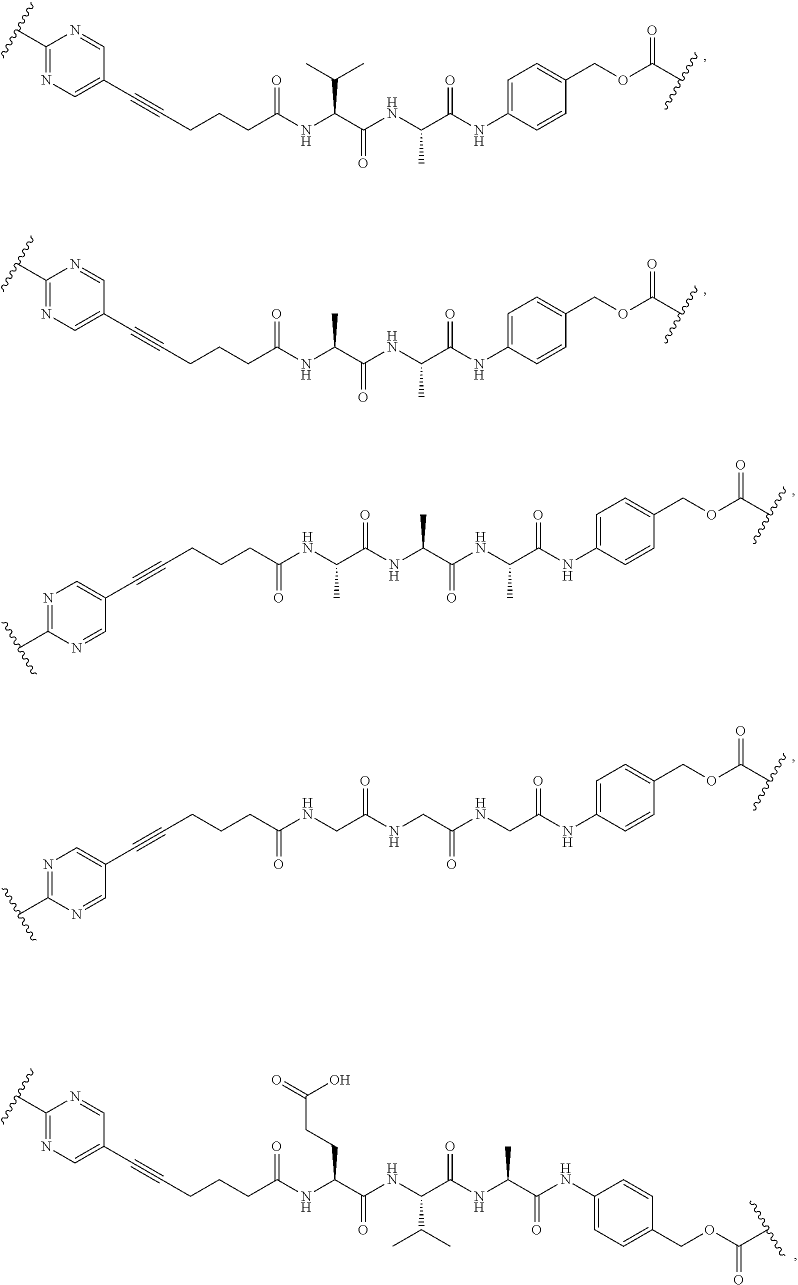 [SMILES: *C(=O)OCc1ccc(NC(=O)[C@H](C)NC(=O)[C@@H](NC(=O)CCCC#Cc2cnc(*)nc2)C(C)C)cc1 ; *C(=O)OCc1ccc(NC(=O)[C@H](C)NC(=O)[C@H](C)NC(=O)CCCC#Cc2cnc(*)nc2)cc1 ; *C(=O)OCc1ccc(NC(=O)[C@H](C)NC(=O)[C@H](C)NC(=O)[C@H](C)NC(=O)CCCC#Cc2cnc(*)nc2)cc1 ; *C(=O)OCc1ccc(NC(=O)CNC(=O)CNC(=O)CNC(=O)CCCC#Cc2cnc(*)nc2)cc1 ; *C(=O)OCc1ccc(NC(=O)[C@H](C)NC(=O)[C@@H](NC(=O)[C@H](CCC(=O)O)NC(=O)CCCC#Cc2cnc(*)nc2)C(C)C)cc1]

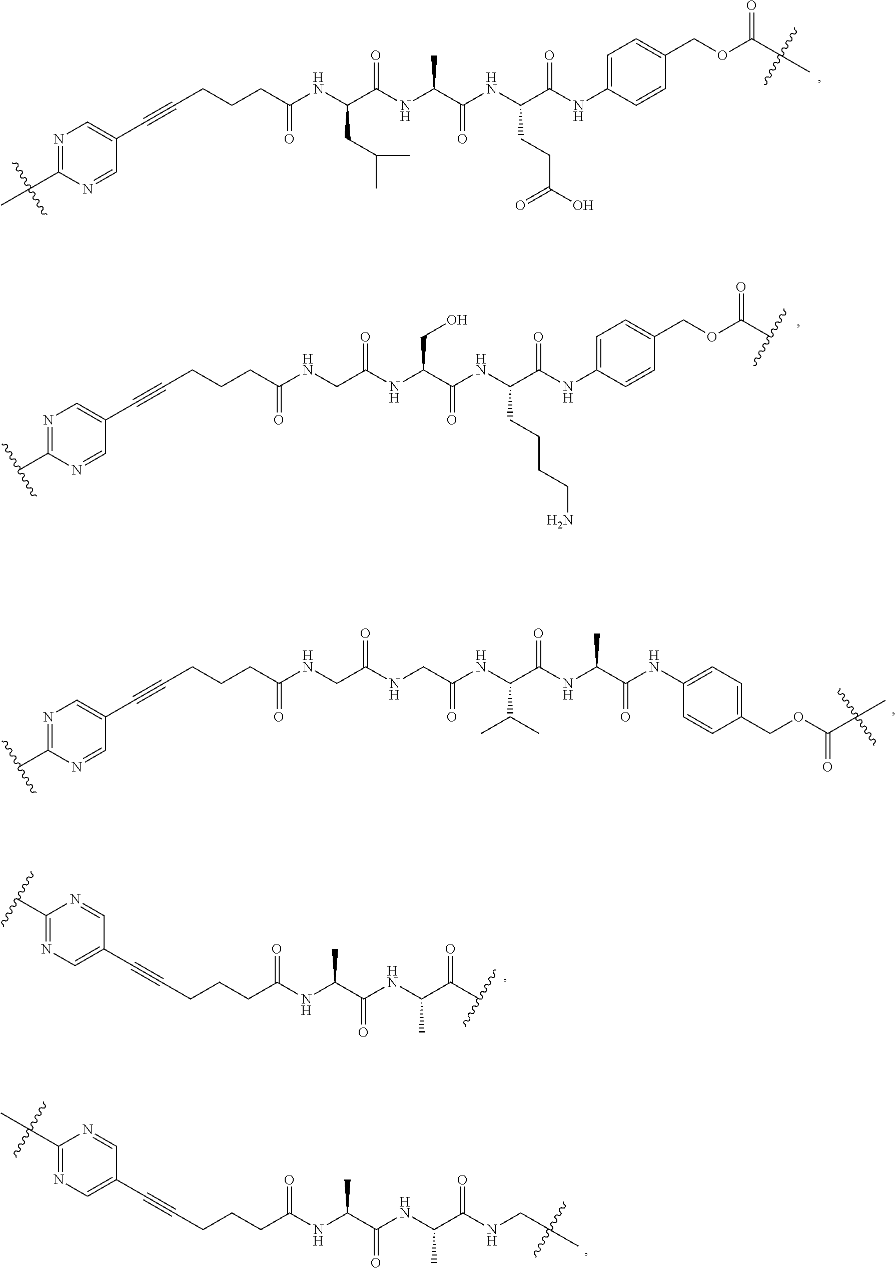 [SMILES: *C(=O)OCc1ccc(NC(=O)[C@H](CCC(=O)O)NC(=O)[C@H](C)NC(=O)[C@@H](CC(C)C)NC(=O)CCCC#Cc2cnc(*)nc2)cc1 ; *C(=O)OCc1ccc(NC(=O)[C@H](CCCCN)NC(=O)[C@H](CO)NC(=O)CNC(=O)CCCC#Cc2cnc(*)nc2)cc1 ; *C(=O)OCc1ccc(NC(=O)[C@H](C)NC(=O)[C@@H](NC(=O)CNC(=O)CNC(=O)CCCC#Cc2cnc(*)nc2)C(C)C)cc1 ; *C(=O)[C@H](C)NC(=O)[C@H](C)NC(=O)CCCC#Cc1cnc(*)nc1 ; *CNC(=O)[C@H](C)NC(=O)[C@H](C)NC(=O)CCCC#Cc1cnc(*)nc1]

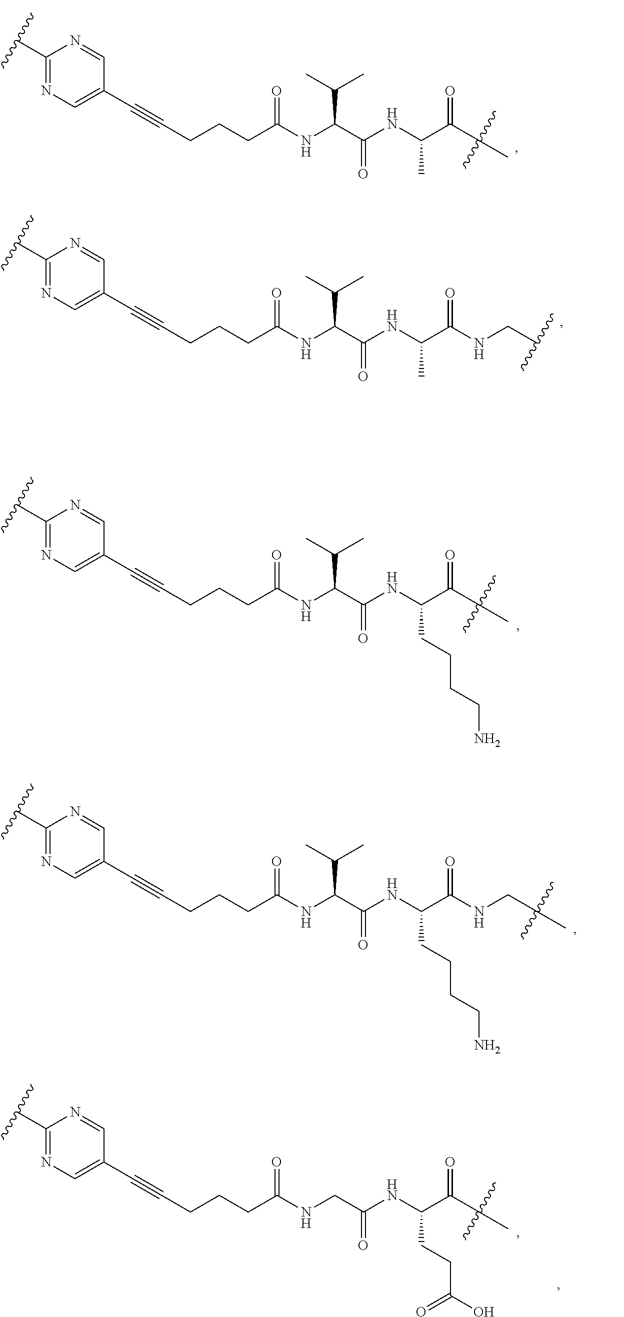 [SMILES: *C(=O)[C@H](C)NC(=O)[C@@H](NC(=O)CCCC#Cc1cnc(*)nc1)C(C)C ; *CNC(=O)[C@H](C)NC(=O)[C@@H](NC(=O)CCCC#Cc1cnc(*)nc1)C(C)C ; *C(=O)[C@H](CCCCN)NC(=O)[C@@H](NC(=O)CCCC#Cc1cnc(*)nc1)C(C)C ; *CNC(=O)[C@H](CCCCN)NC(=O)[C@@H](NC(=O)CCCC#Cc1cnc(*)nc1)C(C)C ; *C(=O)[C@H](CCC(=O)O)NC(=O)CNC(=O)CCCC#Cc1cnc(*)nc1]

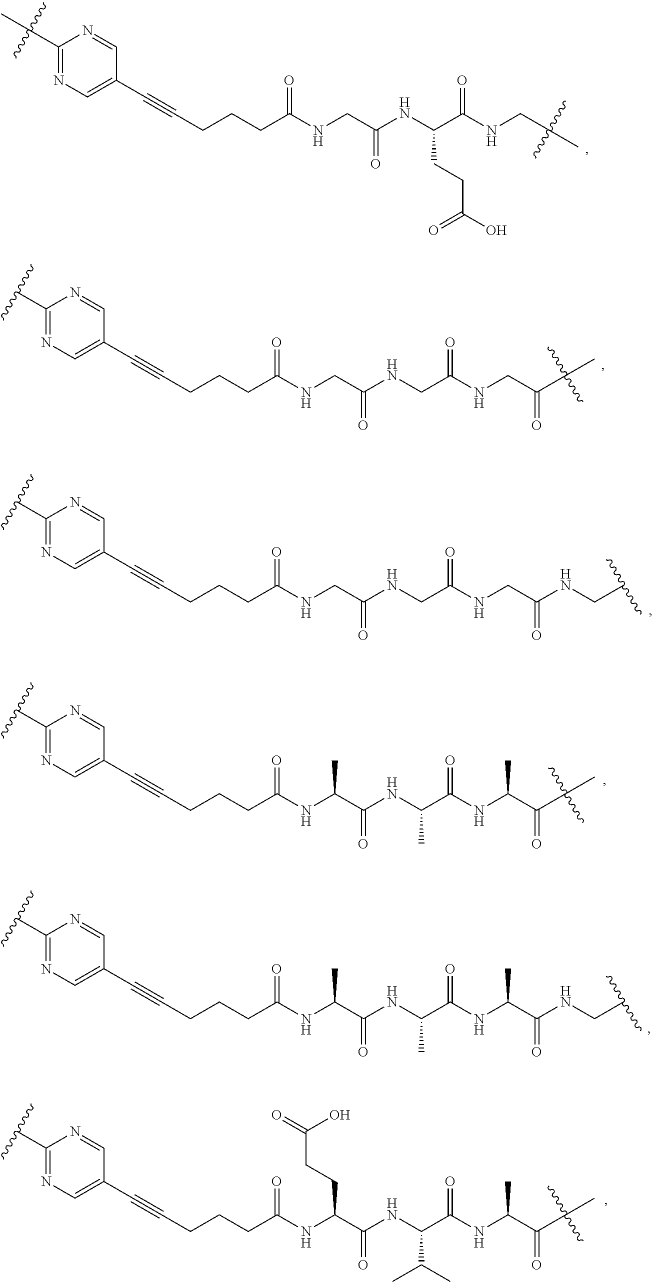 [SMILES: *CNC(=O)[C@H](CCC(=O)O)NC(=O)CNC(=O)CCCC#Cc1cnc(*)nc1 ; *C(=O)CNC(=O)CNC(=O)CNC(=O)CCCC#Cc1cnc(*)nc1 ; *CNC(=O)CNC(=O)CNC(=O)CNC(=O)CCCC#Cc1cnc(*)nc1 ; *C(=O)[C@H](C)NC(=O)[C@H](C)NC(=O)[C@H](C)NC(=O)CCCC#Cc1cnc(*)nc1 ; *CNC(=O)[C@H](C)NC(=O)[C@H](C)NC(=O)[C@H](C)NC(=O)CCCC#Cc1cnc(*)nc1 ; *C(=O)[C@H](C)NC(=O)[C@@H](NC(=O)[C@H](CCC(=O)O)NC(=O)CCCC#Cc1cnc(*)nc1)C(C)C]

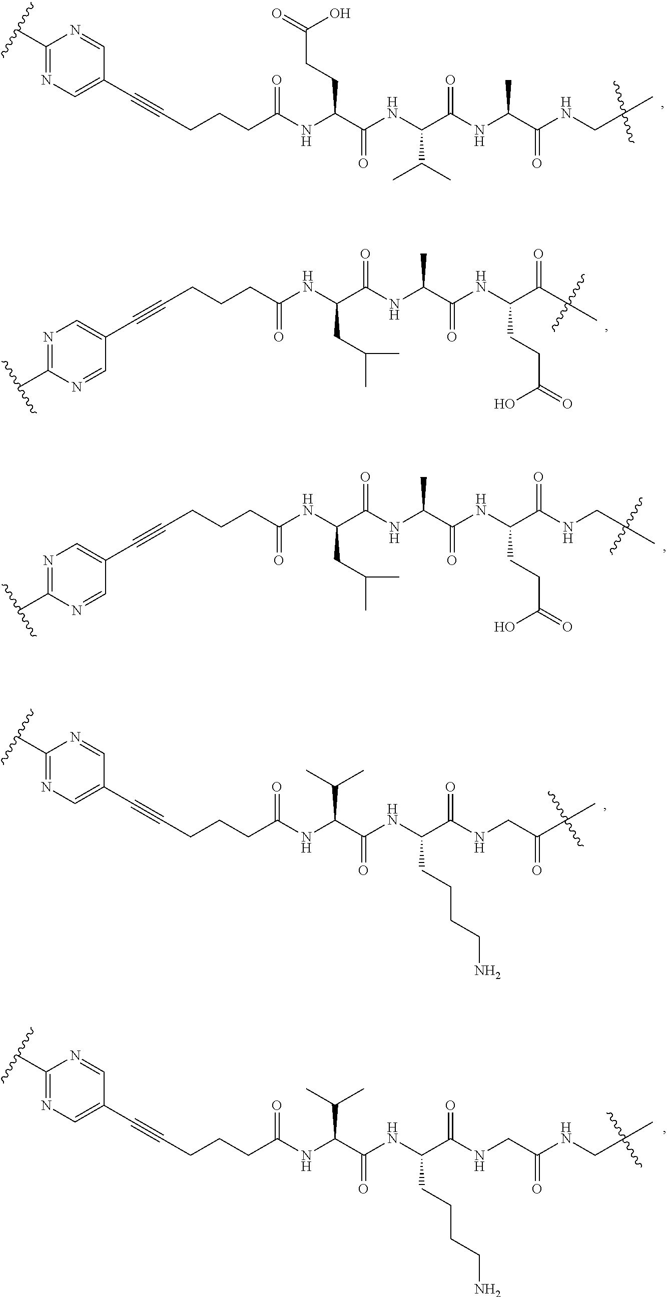 [SMILES: *CNC(=O)[C@H](C)NC(=O)[C@@H](NC(=O)[C@H](CCC(=O)O)NC(=O)CCCC#Cc1cnc(*)nc1)C(C)C ; *C(=O)[C@H](CCC(=O)O)NC(=O)[C@H](C)NC(=O)[C@@H](CC(C)C)NC(=O)CCCC#Cc1cnc(*)nc1 ; *CNC(=O)[C@H](CCC(=O)O)NC(=O)[C@H](C)NC(=O)[C@@H](CC(C)C)NC(=O)CCCC#Cc1cnc(*)nc1 ; *C(=O)CNC(=O)[C@H](CCCCN)NC(=O)[C@@H](NC(=O)CCCC#Cc1cnc(*)nc1)C(C)C ; *CNC(=O)CNC(=O)[C@H](CCCCN)NC(=O)[C@@H](NC(=O)CCCC#Cc1cnc(*)nc1)C(C)C]

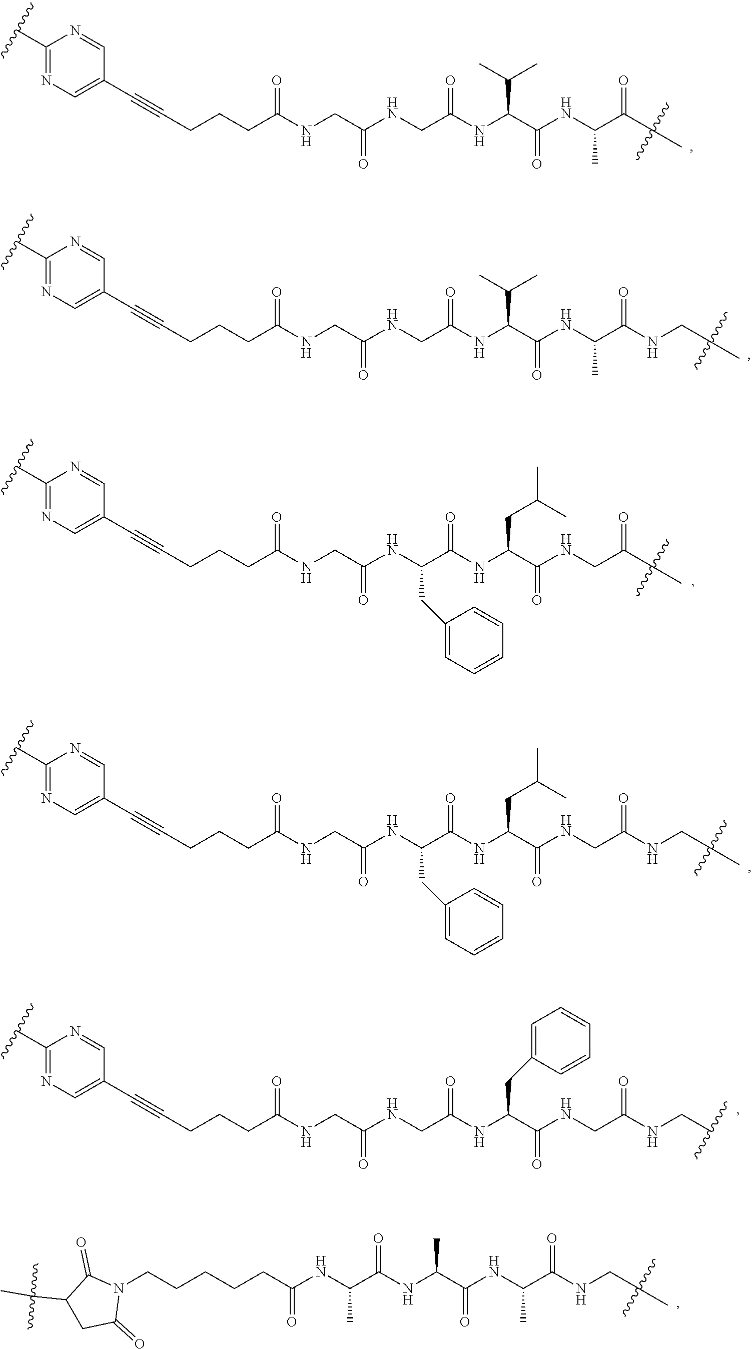 [SMILES: *C(=O)[C@H](C)NC(=O)[C@@H](NC(=O)CNC(=O)CNC(=O)CCCC#Cc1cnc(*)nc1)C(C)C ; *CNC(=O)[C@H](C)NC(=O)[C@@H](NC(=O)CNC(=O)CNC(=O)CCCC#Cc1cnc(*)nc1)C(C)C ; *C(=O)CNC(=O)[C@H](CC(C)C)NC(=O)[C@H](Cc1ccccc1)NC(=O)CNC(=O)CCCC#Cc1cnc(*)nc1 ; *CNC(=O)CNC(=O)[C@H](CC(C)C)NC(=O)[C@H](Cc1ccccc1)NC(=O)CNC(=O)CCCC#Cc1cnc(*)nc1 ; *CNC(=O)CNC(=O)[C@H](Cc1ccccc1)NC(=O)CNC(=O)CNC(=O)CCCC#Cc1cnc(*)nc1 ; *CNC(=O)[C@H](C)NC(=O)[C@H](C)NC(=O)[C@H](C)NC(=O)CCCCCN1C(=O)CC(*)C1=O]

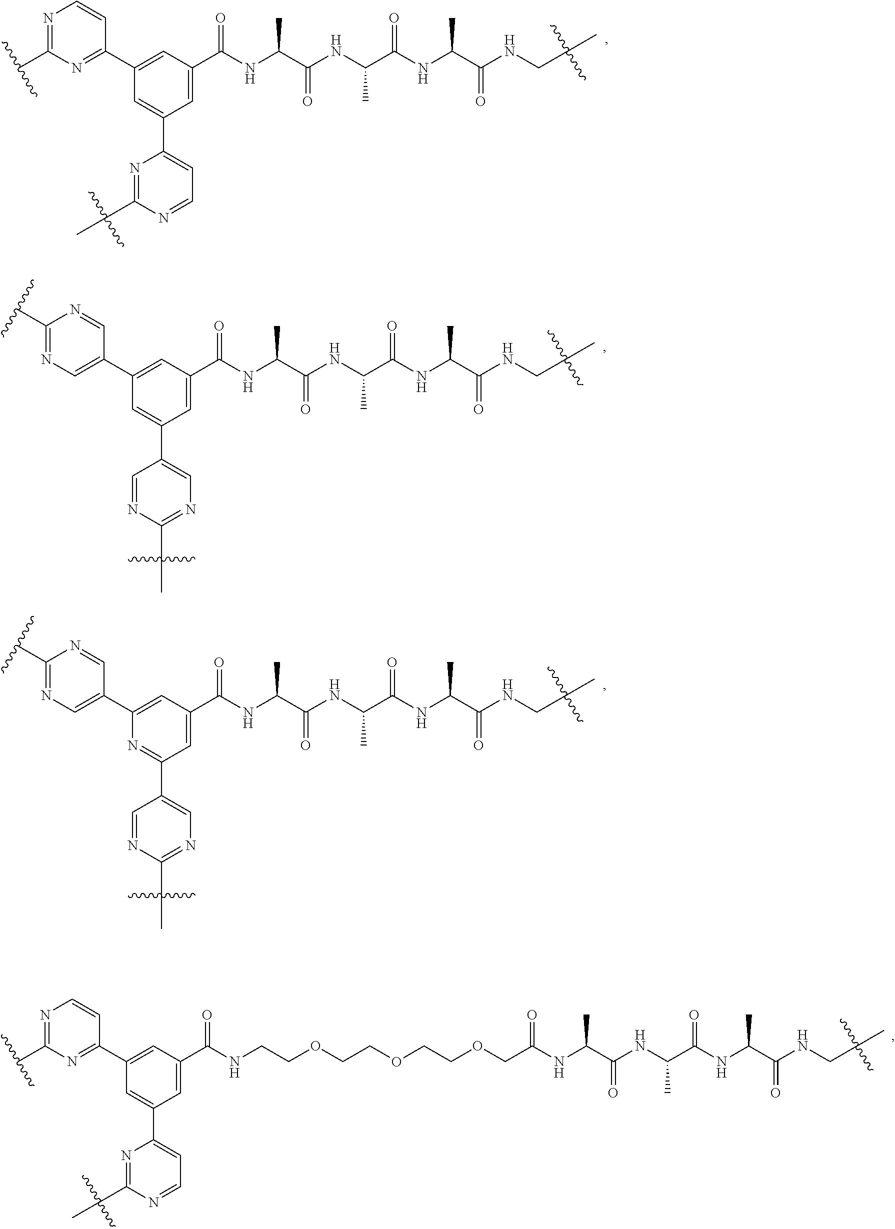 [SMILES: *CNC(=O)[C@H](C)NC(=O)[C@H](C)NC(=O)[C@H](C)NC(=O)c1cc(-c2ccnc(*)n2)cc(-c2ccnc(*)n2)c1 ; *CNC(=O)[C@H](C)NC(=O)[C@H](C)NC(=O)[C@H](C)NC(=O)c1cc(-c2cnc(*)nc2)cc(-c2cnc(*)nc2)c1 ; *CNC(=O)[C@H](C)NC(=O)[C@H](C)NC(=O)[C@H](C)NC(=O)c1cc(-c2cnc(*)nc2)nc(-c2cnc(*)nc2)c1 ; *CNC(=O)[C@H](C)NC(=O)[C@H](C)NC(=O)[C@H](C)NC(=O)COCCOCCOCCNC(=O)c1cc(-c2ccnc(*)n2)cc(-c2ccnc(*)n2)c1]

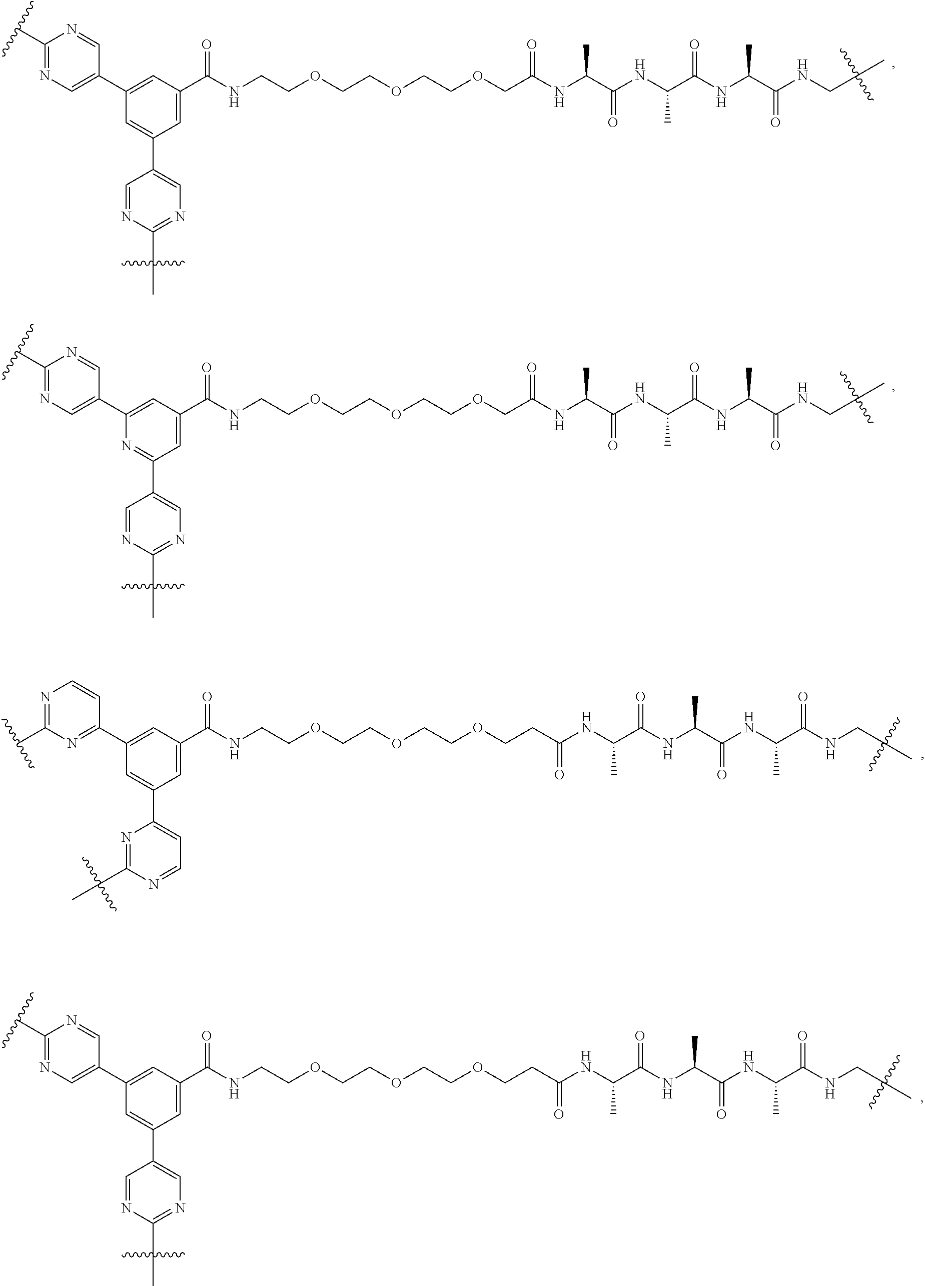 [SMILES: *CNC(=O)[C@H](C)NC(=O)[C@H](C)NC(=O)[C@H](C)NC(=O)COCCOCCOCCNC(=O)c1cc(-c2cnc(*)nc2)cc(-c2cnc(*)nc2)c1 ; *CNC(=O)[C@H](C)NC(=O)[C@H](C)NC(=O)[C@H](C)NC(=O)COCCOCCOCCNC(=O)c1cc(-c2cnc(*)nc2)nc(-c2cnc(*)nc2)c1 ; *CNC(=O)[C@H](C)NC(=O)[C@H](C)NC(=O)[C@H](C)NC(=O)CCOCCOCCOCCNC(=O)c1cc(-c2ccnc(*)n2)cc(-c2ccnc(*)n2)c1 ; *CNC(=O)[C@H](C)NC(=O)[C@H](C)NC(=O)[C@H](C)NC(=O)CCOCCOCCOCCNC(=O)c1cc(-c2cnc(*)nc2)cc(-c2cnc(*)nc2)c1]

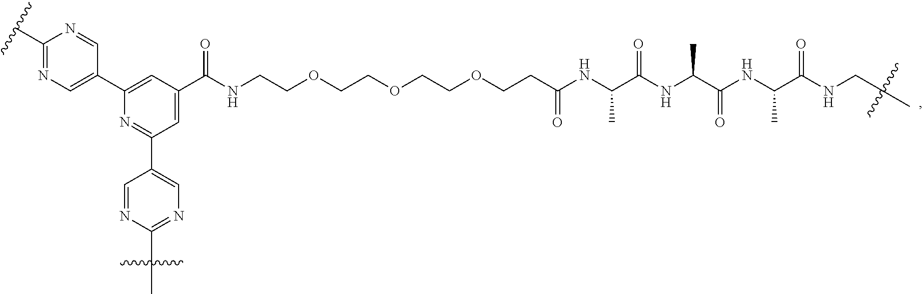 [SMILES: *CNC(=O)[C@H](C)NC(=O)[C@H](C)NC(=O)[C@H](C)NC(=O)CCOCCOCCOCCNC(=O)c1cc(-c2cnc(*)nc2)nc(-c2cnc(*)nc2)c1]

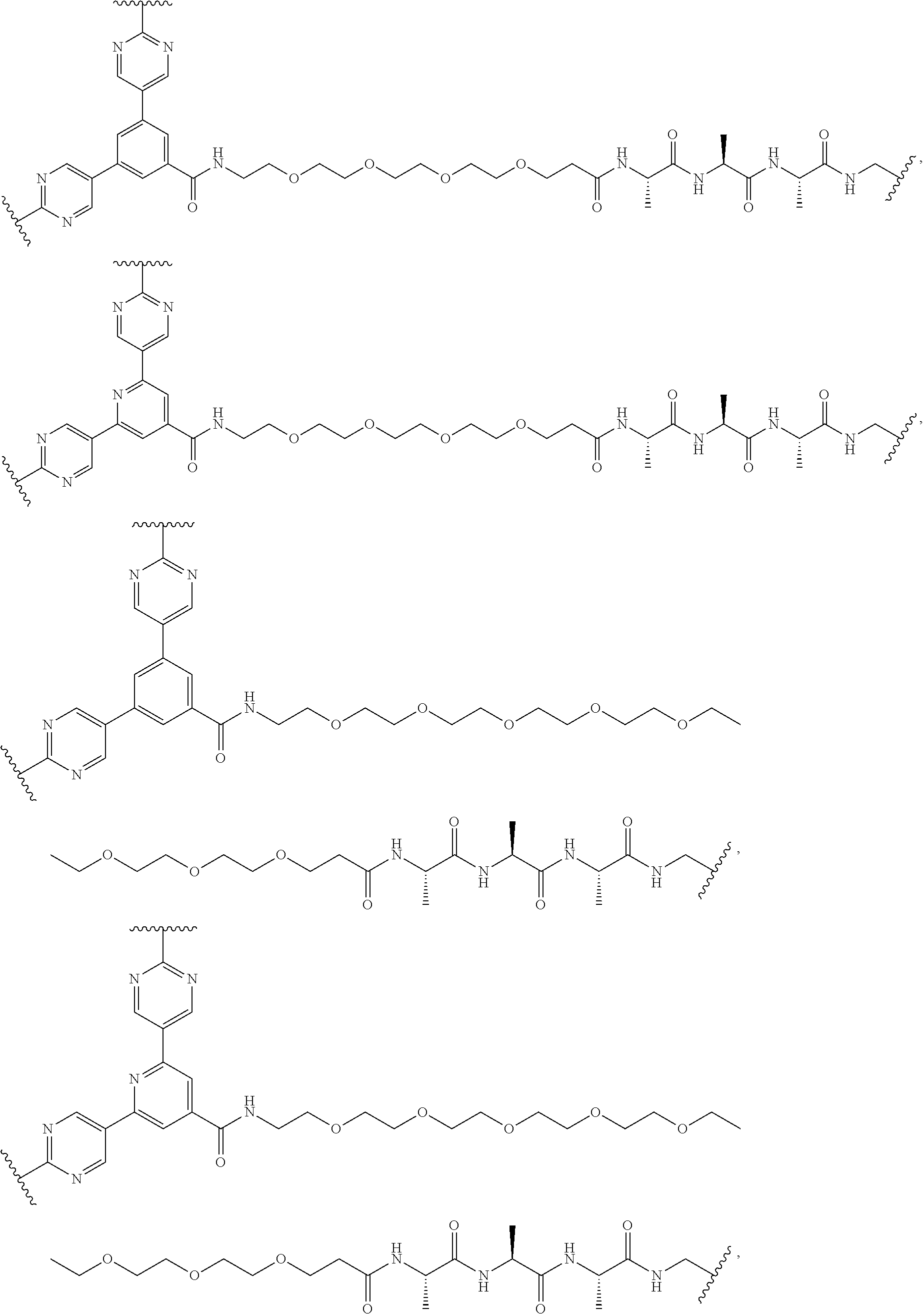

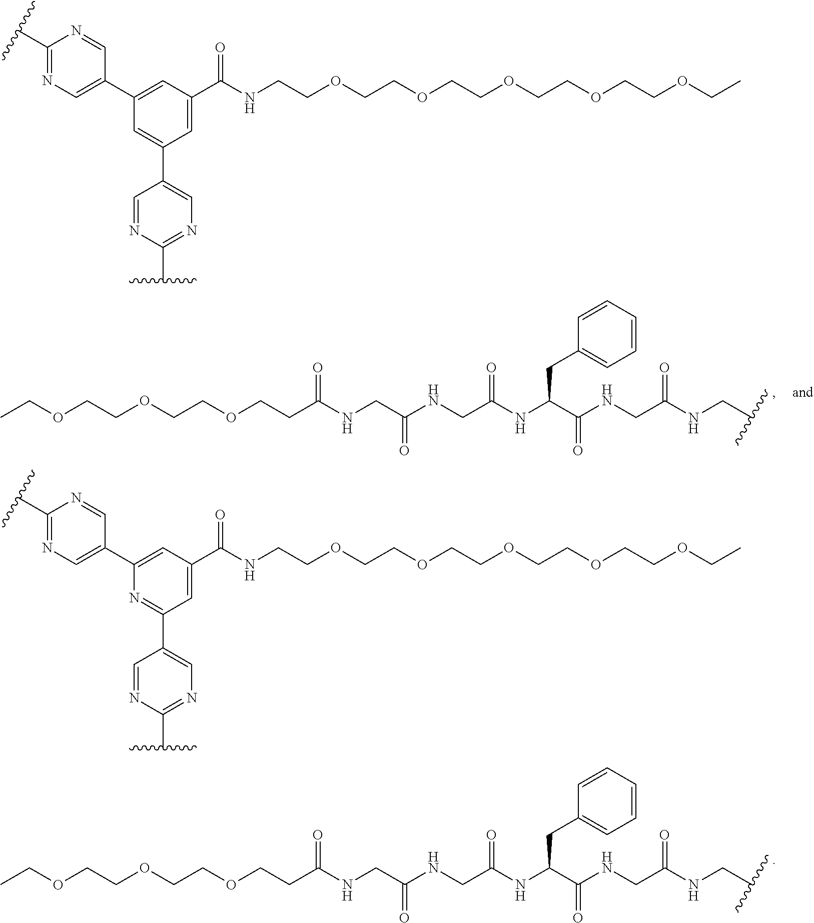

In some embodiments,

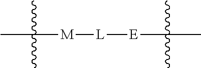

is selected from the following structures:

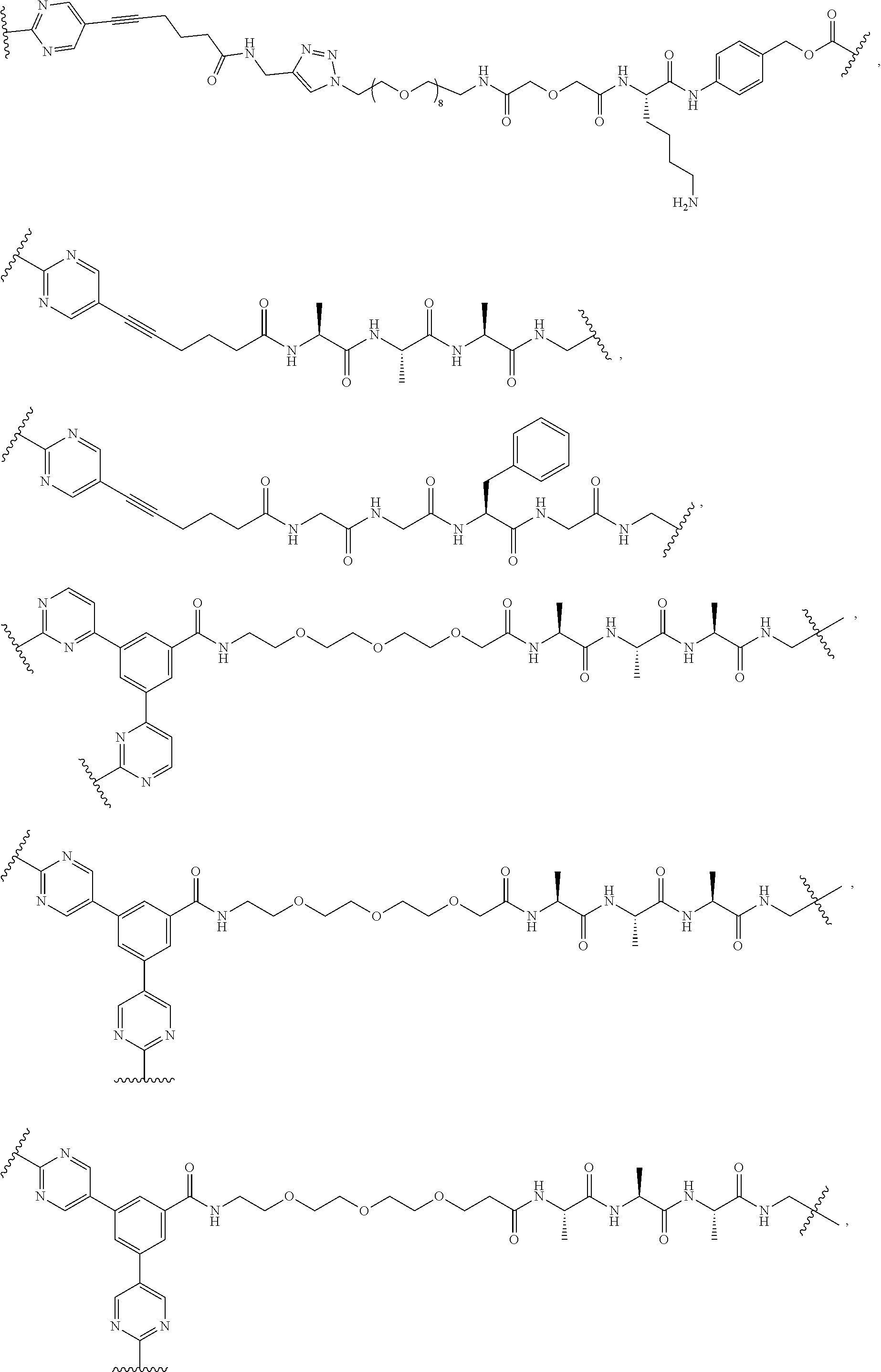

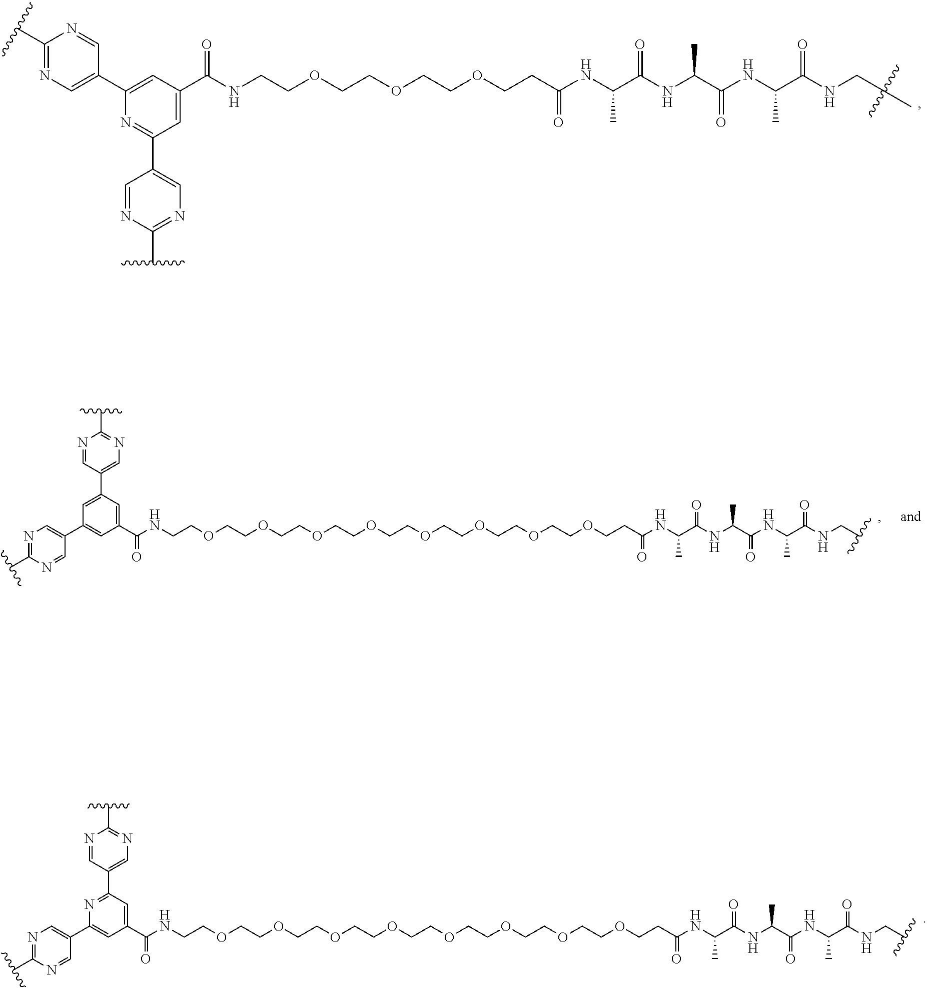 [SMILES: *CNC(=O)[C@H](C)NC(=O)[C@H](C)NC(=O)[C@H](C)NC(=O)CCOCCOCCOCCNC(=O)c1cc(-c2cnc(*)nc2)nc(-c2cnc(*)nc2)c1 ; *CNC(=O)[C@H](C)NC(=O)[C@H](C)NC(=O)[C@H](C)NC(=O)CCOCCOCCOCCOCCOCCOCCOCCOCCNC(=O)c1cc(-c2cnc(*)nc2)cc(-c2cnc(*)nc2)c1 ; *CNC(=O)[C@H](C)NC(=O)[C@H](C)NC(=O)[C@H](C)NC(=O)CCOCCOCCOCCOCCOCCOCCOCCOCCNC(=O)c1cc(-c2cnc(*)nc2)nc(-c2cnc(*)nc2)c1]

In some embodiments, the cytotoxic drug of an ADC provided herein is selected from the group consisting of tubulin inhibitors, DNA intercalators, DNA topoisomerase inhibitors, and RNA polymerase inhibitors. In certain embodiments, the tubulin inhibitor is an auristatin or maytansinoid. In certain embodiments, the DNA intercalator is pyrrolobenzodiazepine (PBD). In certain embodiments, the DNA topoisomerase inhibitor is a topoisomerase I inhibitor (for example, camptothecin, hydroxycamptothecin, 9-aminocamptothecin, SN-38, irinotecan, topotecan, bellotecan, or rubotecan) or a topoisomerase II inhibitor (for example, Doxorubicin, PNU-159682, docarmicin, daunorubicin, mitoxantrone, podophyllotoxin, or etoposide). In certain embodiments, the RNA polymerase inhibitor is α-amanitine (α-amanitin) or a pharmaceutically acceptable salt, ester or analog thereof.

In some embodiments, the cytotoxic drug is selected from the group consisting of compounds represented by Formula I and Formula II, or pharmaceutically acceptable salts, esters, stereoisomers, and tautomers of compounds represented by Formula I and Formula II or prodrug of said compounds:

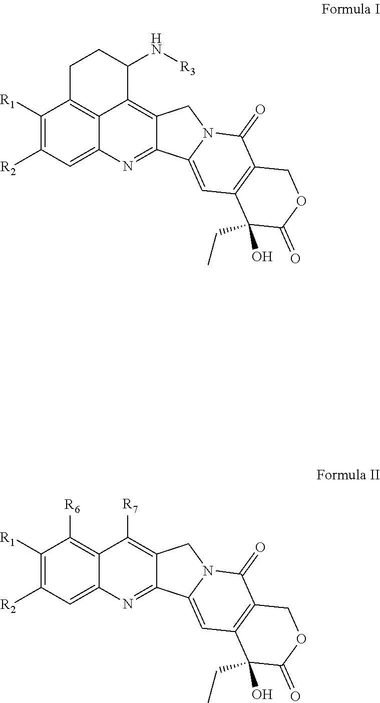

wherein R₁ and R₂ are each independently selected from C₁₋₆ alkyl and halogen; R₃ is selected from H and —CO—CH₂OH; R₄ and R₅ are each independently selected from H, halogen, and hydroxyl; or R₄ and R₅ are connected to the associated carbon atoms to form a 5-6 membered oxygen-containing heterocyclic ring; R₆ is selected from hydrogen or —C₁₋₄ alkylene-NRₐR_{b}, —C₁₋₄ alkylene-SiRₐR_{b}R_{c}, —SiRₐR_{b}R_{c}, —C₁₋₄ alkylene=N—ORₐ; and R₇ is selected from C₁₋₆ alkyl, —C₁₋₄ alkylene-NRₐR_{b}; wherein, Rₐ, R_{b}, and R_{c} are independently selected from H, C₁₋₆ alkyl, —SO₂—C₁₋₆ alkyl, and —CO—C₁₋₆ alkyl at each occurrence; wherein optionally Rₐ and R_{b} connected to the associated atoms form a 5-6 membered nitrogen containing heterocyclic ring.

In some embodiments, the cytotoxic drug is selected from the following compounds or pharmaceutically acceptable salts, esters, stereoisomers, tautomers or prodrugs of said compounds:

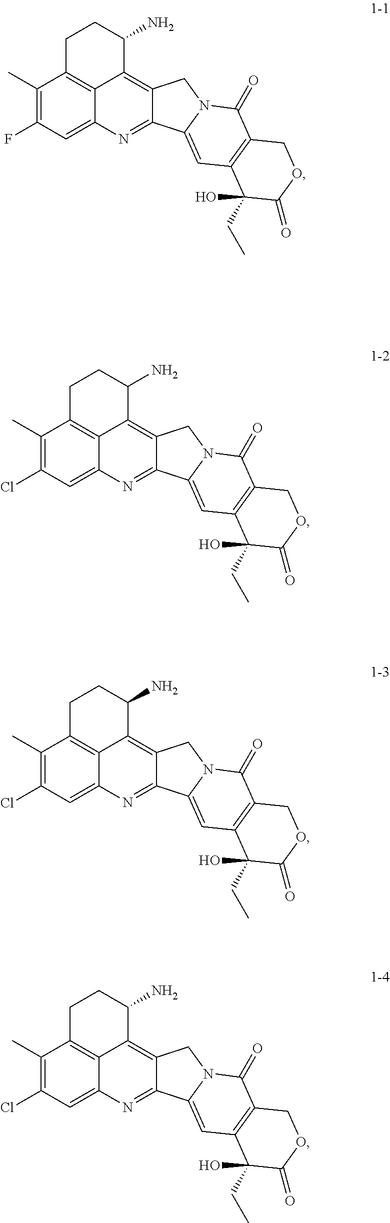 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3]

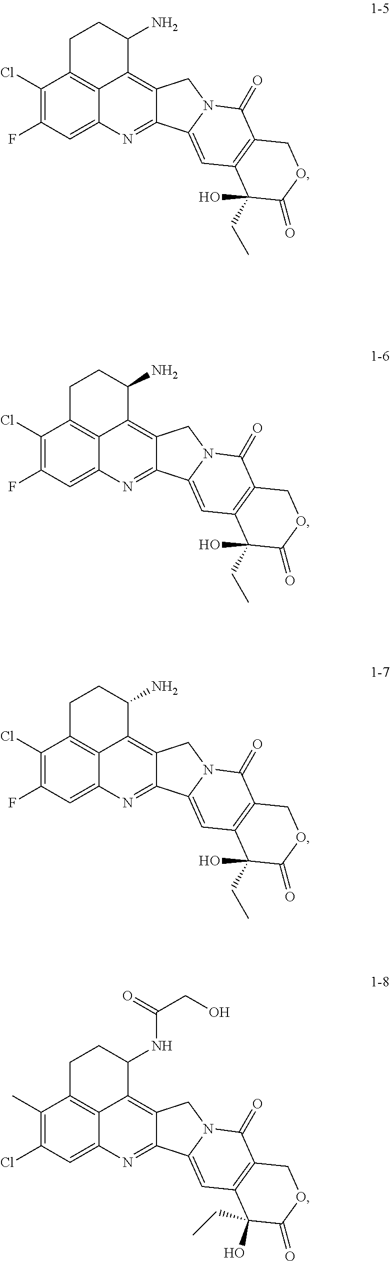 [SMILES: CC[C@@]1(O)C(=O)OCc2c1cc1n(c2=O)Cc2c-1nc1cc(F)c(Cl)c3c1c2C(N)CC3 ; CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3]

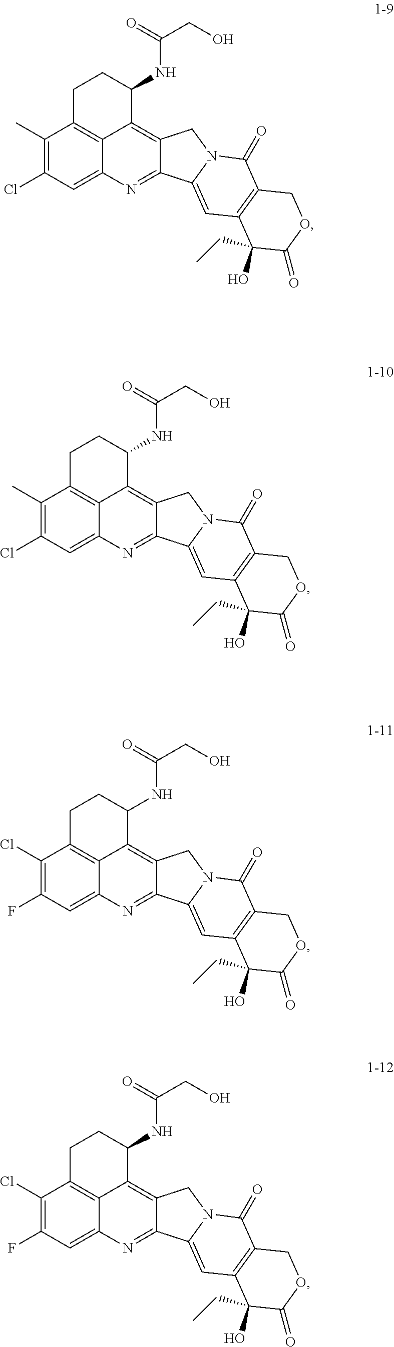 [SMILES: CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3]

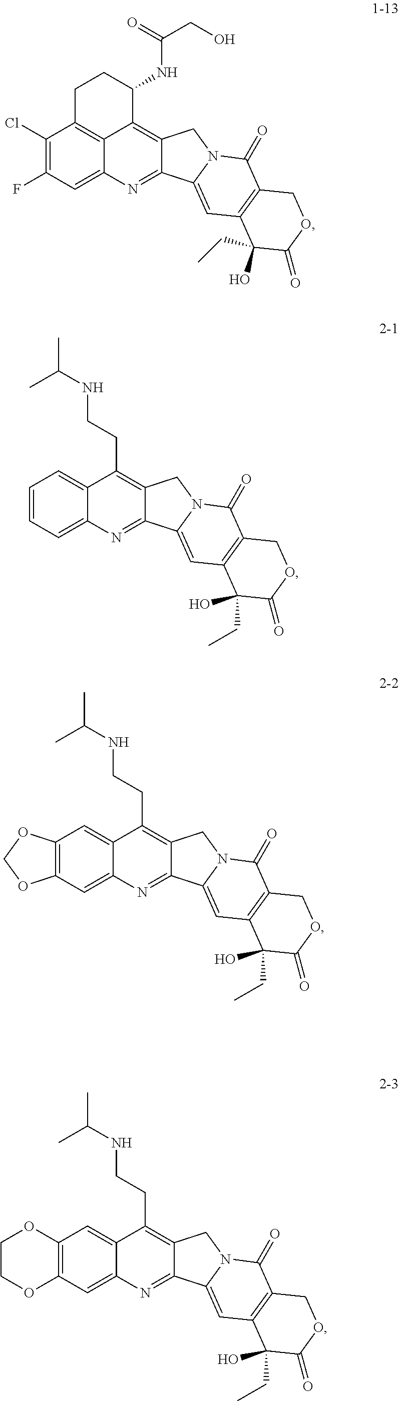 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1cc3c(cc1c2CCNC(C)C)OCCO3]

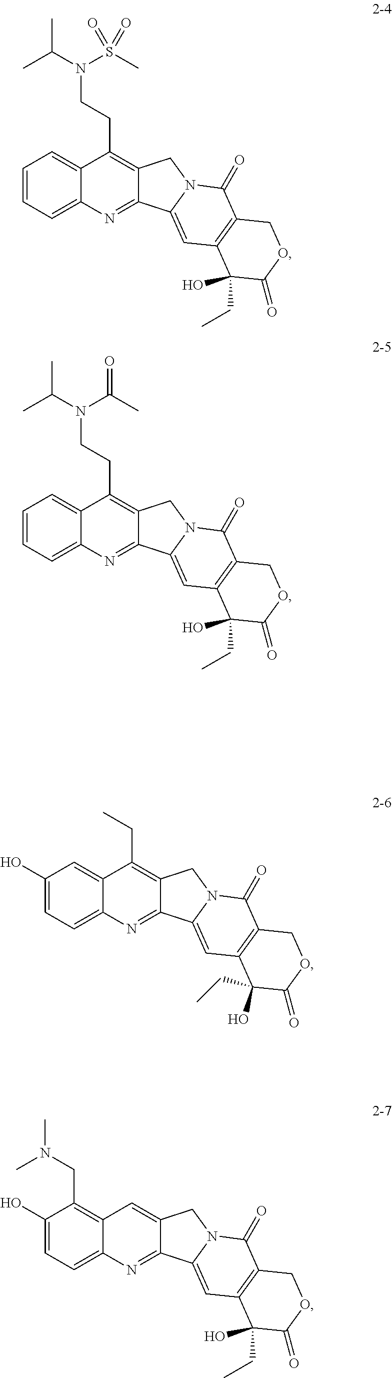 [SMILES: CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1ccccc1c2CCN(C(C)=O)C(C)C ; CCc1c2c(nc3ccc(O)cc13)-c1cc3c(c(=O)n1C2)COC(=O)[C@]3(O)CC ; CC[C@@]1(O)C(=O)OCc2c1cc1n(c2=O)Cc2cc3c(CN(C)C)c(O)ccc3nc2-1]

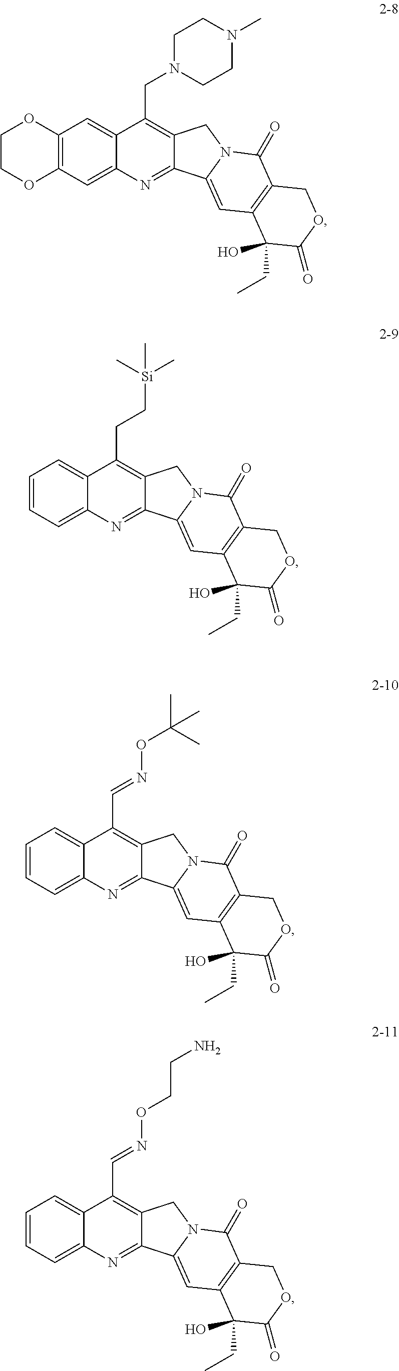

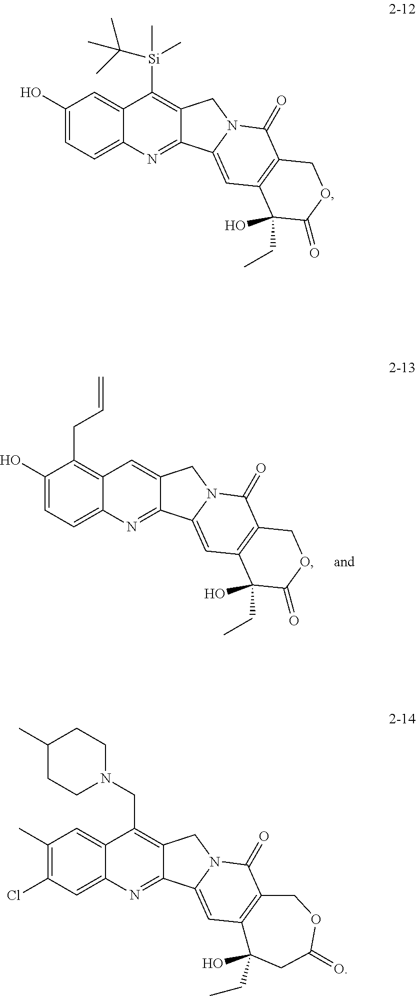 [SMILES: CC[C@@]1(O)C(=O)OCc2c1cc1n(c2=O)Cc2c-1nc1ccc(O)cc1c2[Si](C)(C)C(C)(C)C ; C=CCc1c(O)ccc2nc3c(cc12)Cn1c-3cc2c(c1=O)COC(=O)[C@]2(O)CC ; CC[C@@]1(O)CC(=O)OCc2c1cc1n(c2=O)Cc2c-1nc1cc(Cl)c(C)cc1c2CN1CCC(C)CC1]

Cytotoxic drugs of the ADCs provided herein may contain a variety of functional groups, such as hydroxyl (—OH), carboxyl (—COOH), sulfhydryl (—SH), primary amine (—NH₂), secondary amine (—NR_{A}H) or tertiary amine group (—NR_{B}R_{C}), wherein R_{A}, R_{B} and R_{C} represent non-hydrogen substituents on N, and the cytotoxic drug can be combined with the conjugate through these functional groups.

In some embodiments, the cytotoxic drug is selected from the following compounds or pharmaceutically acceptable salts, esters, stereoisomers, tautomers or prodrugs of said compounds:

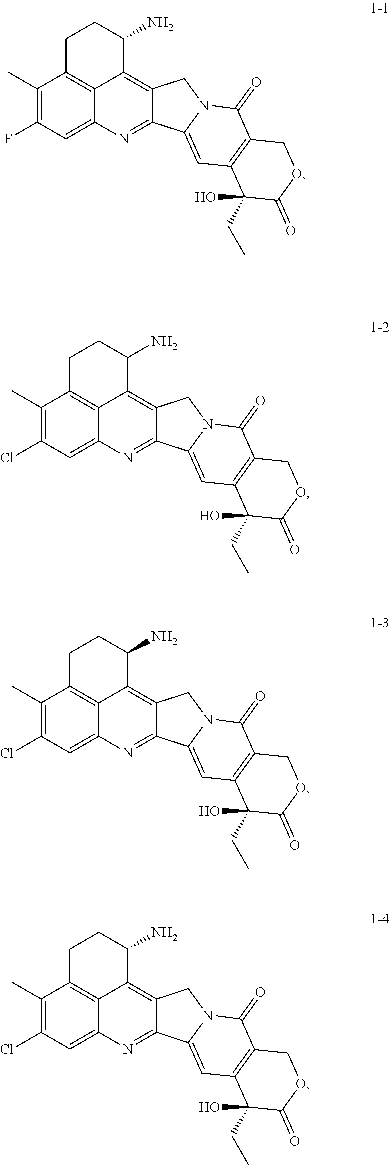 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3]

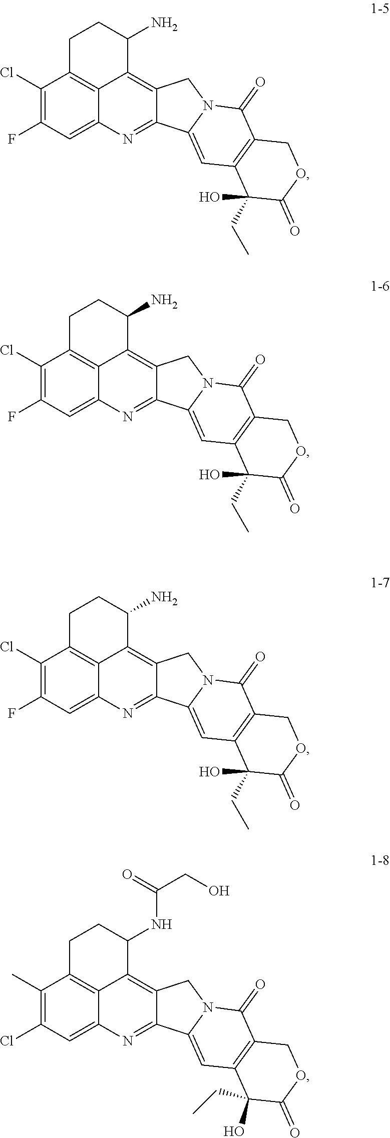 [SMILES: CC[C@@]1(O)C(=O)OCc2c1cc1n(c2=O)Cc2c-1nc1cc(F)c(Cl)c3c1c2C(N)CC3 ; CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3]

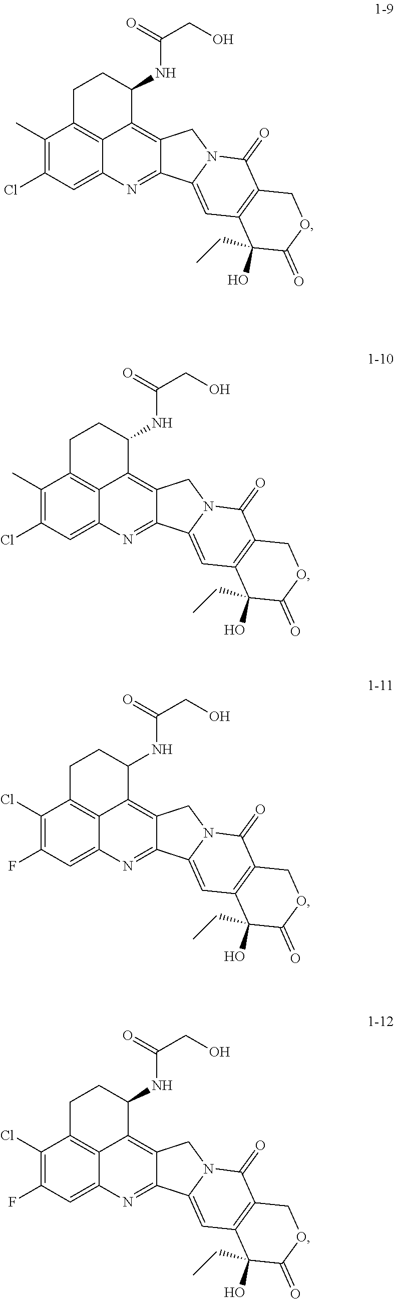 [SMILES: CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3]

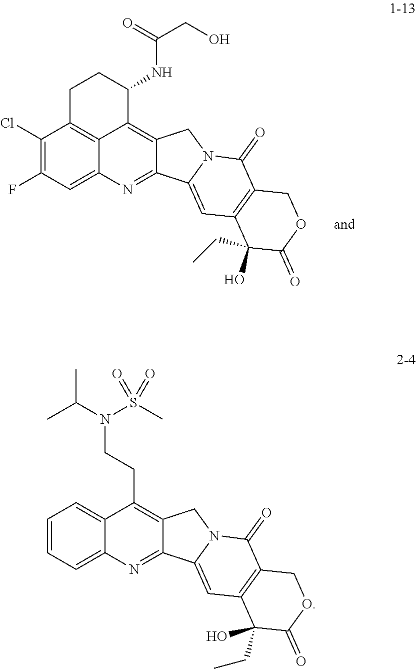 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1ccccc1c2CCN(C(C)C)S(C)(=O)=O]

### Connection Between Linker and Drug (Payload)

In some embodiments, the cytotoxic drug of an ADC provided herein is linked to E in the ADC through an —OH, an —SH, a primary amino group, a secondary amine group, or a tertiary amine group thereon.

In some embodiments, D is a monovalent structure obtained by losing one H from an —OH, —an NH₂, or a secondary amine group on the cytotoxic drug.

### Exemplary ADCs

In some embodiments, an ADC provided herein is selected from the group consisting of ADC A-01 to ADC A-34, the group consisting of ADC B-01 to ADC B-07, or the group consisting of ADC C-01 to ADC C-28, wherein the structures of the ADCs are as follows:

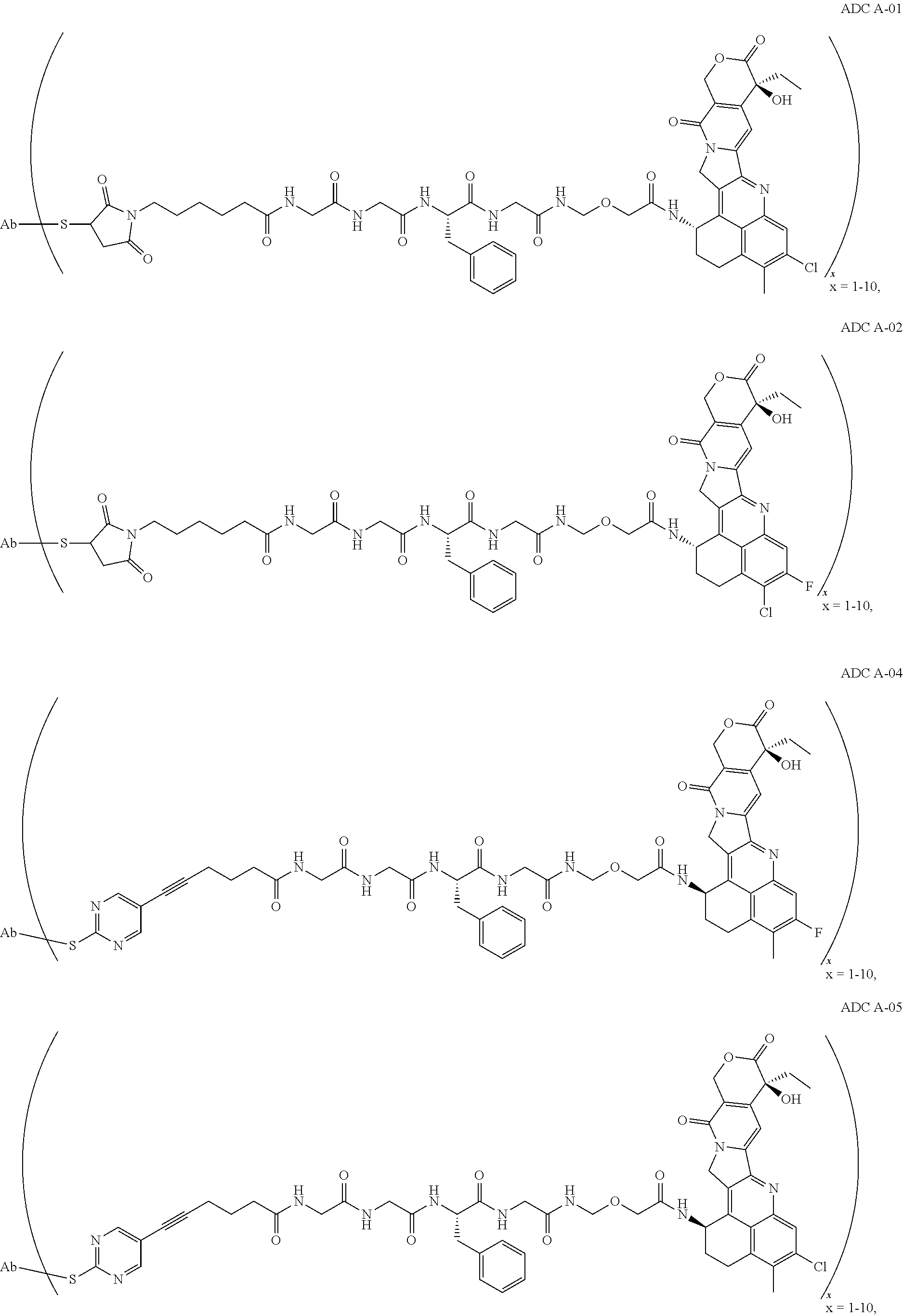

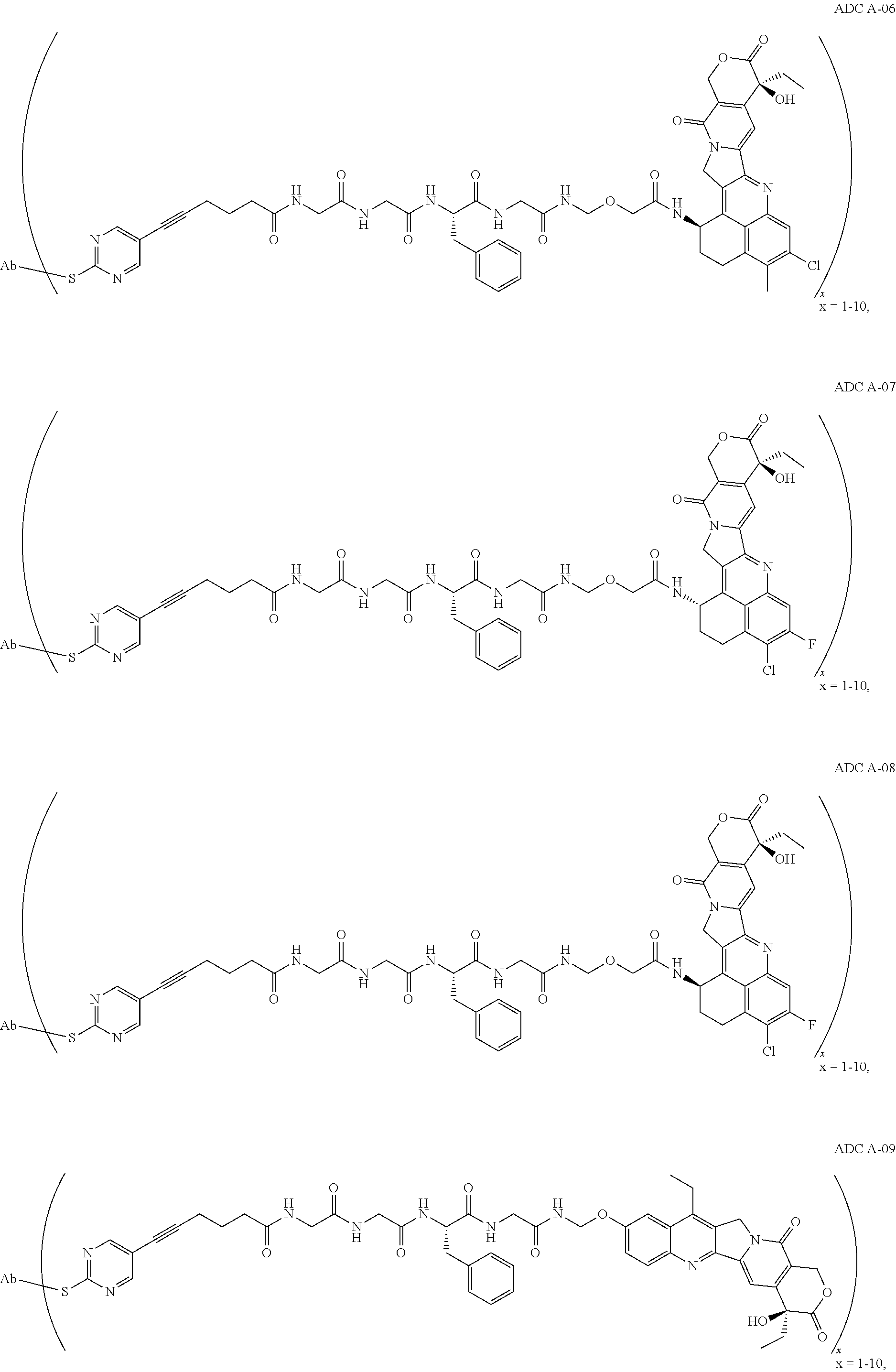

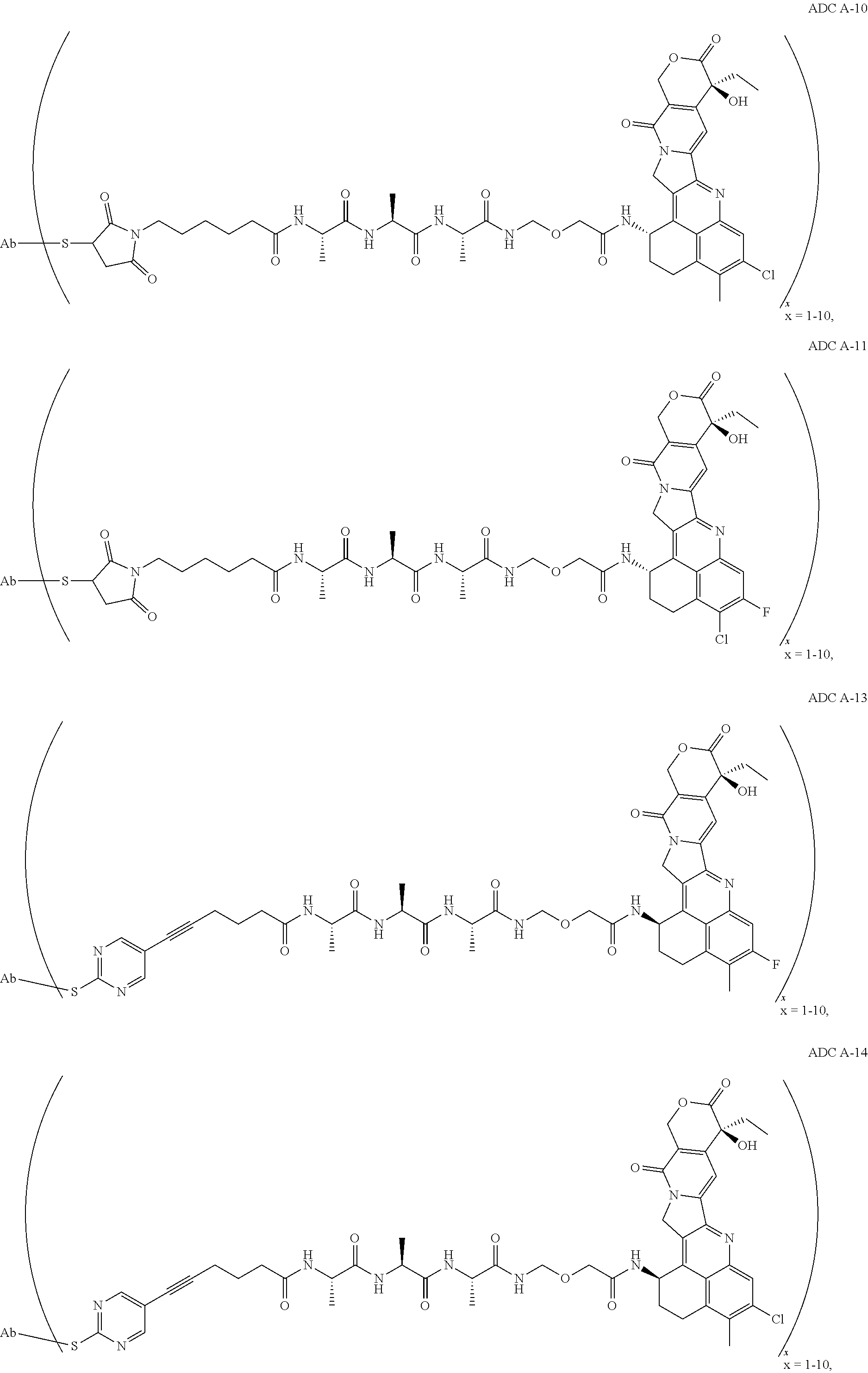

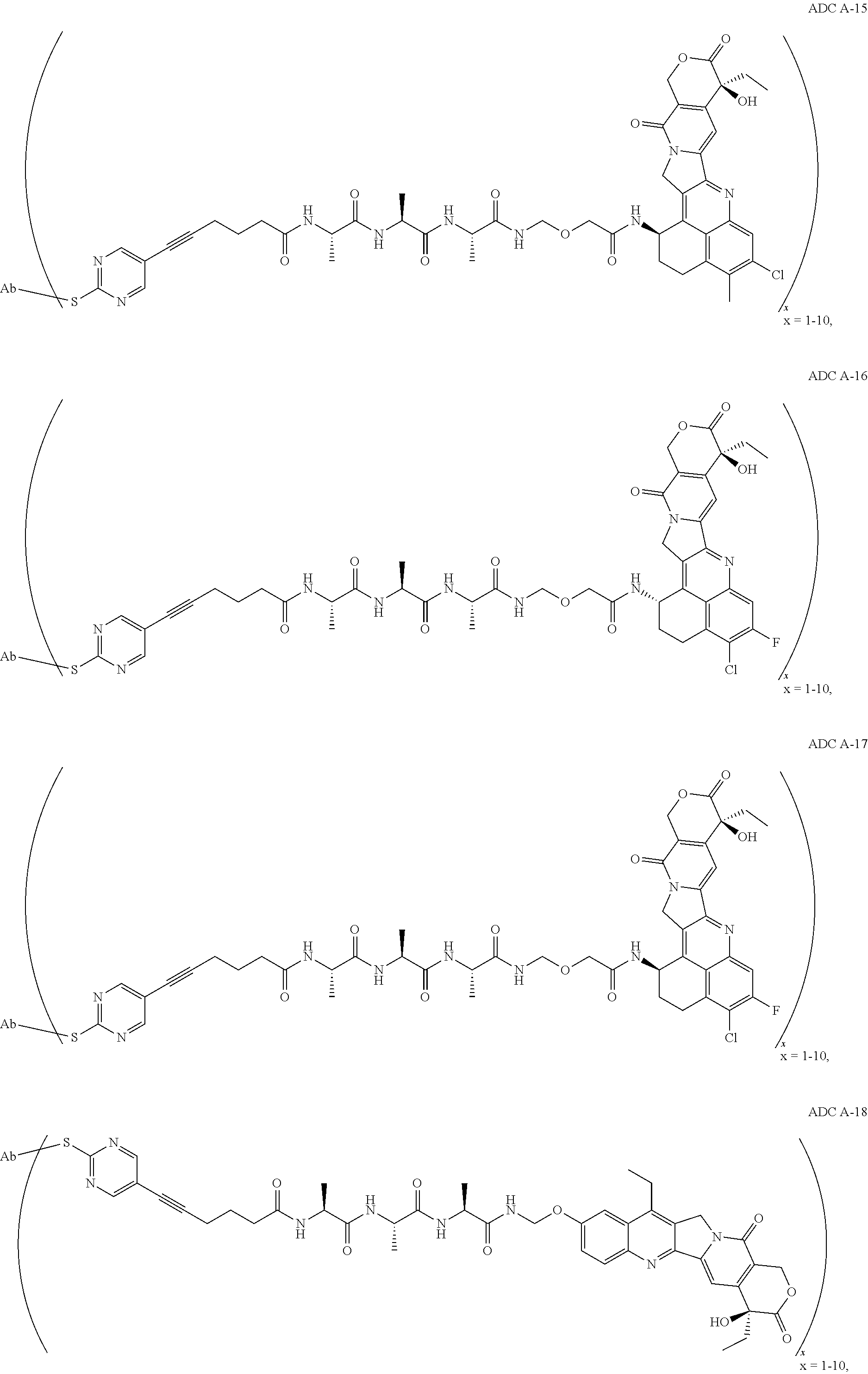

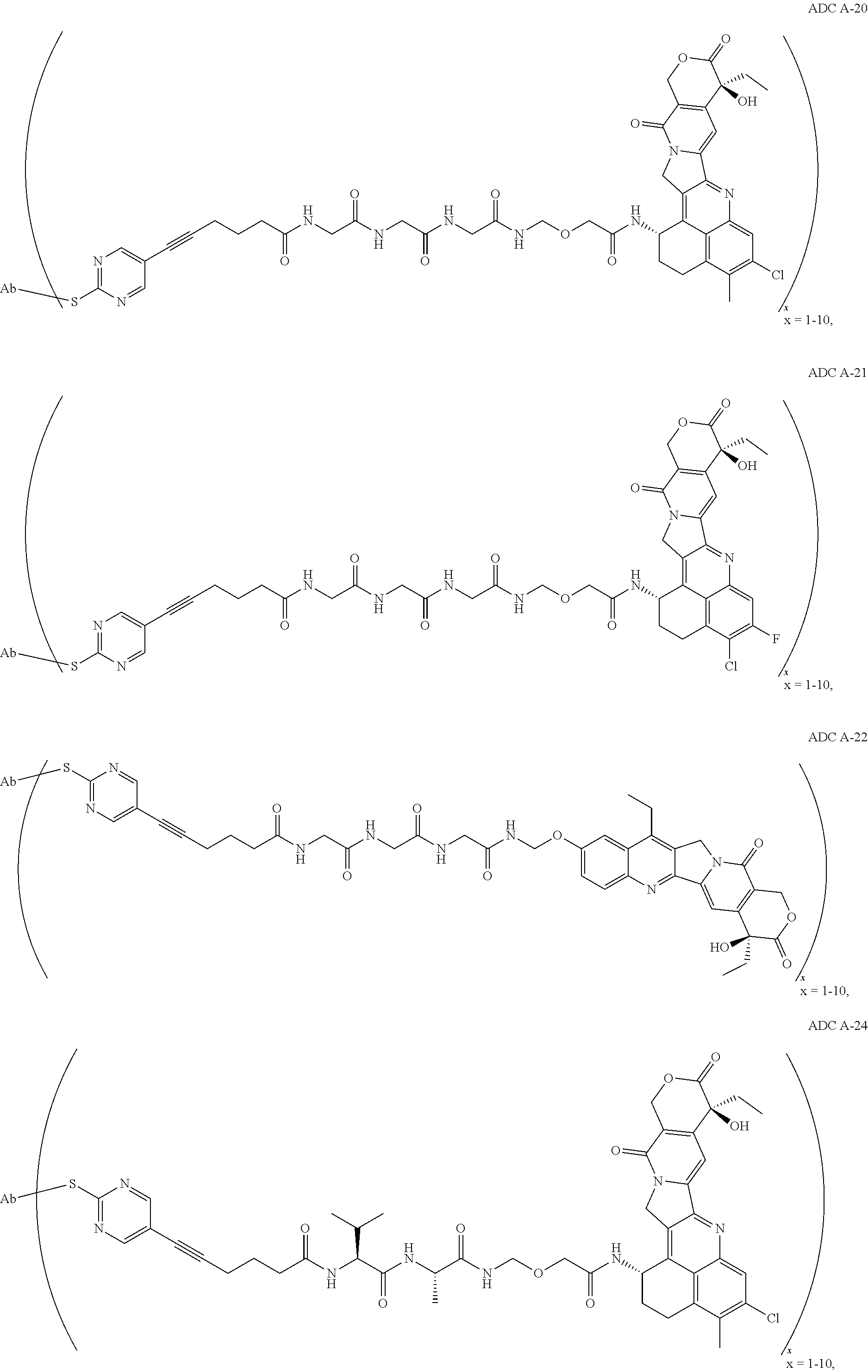

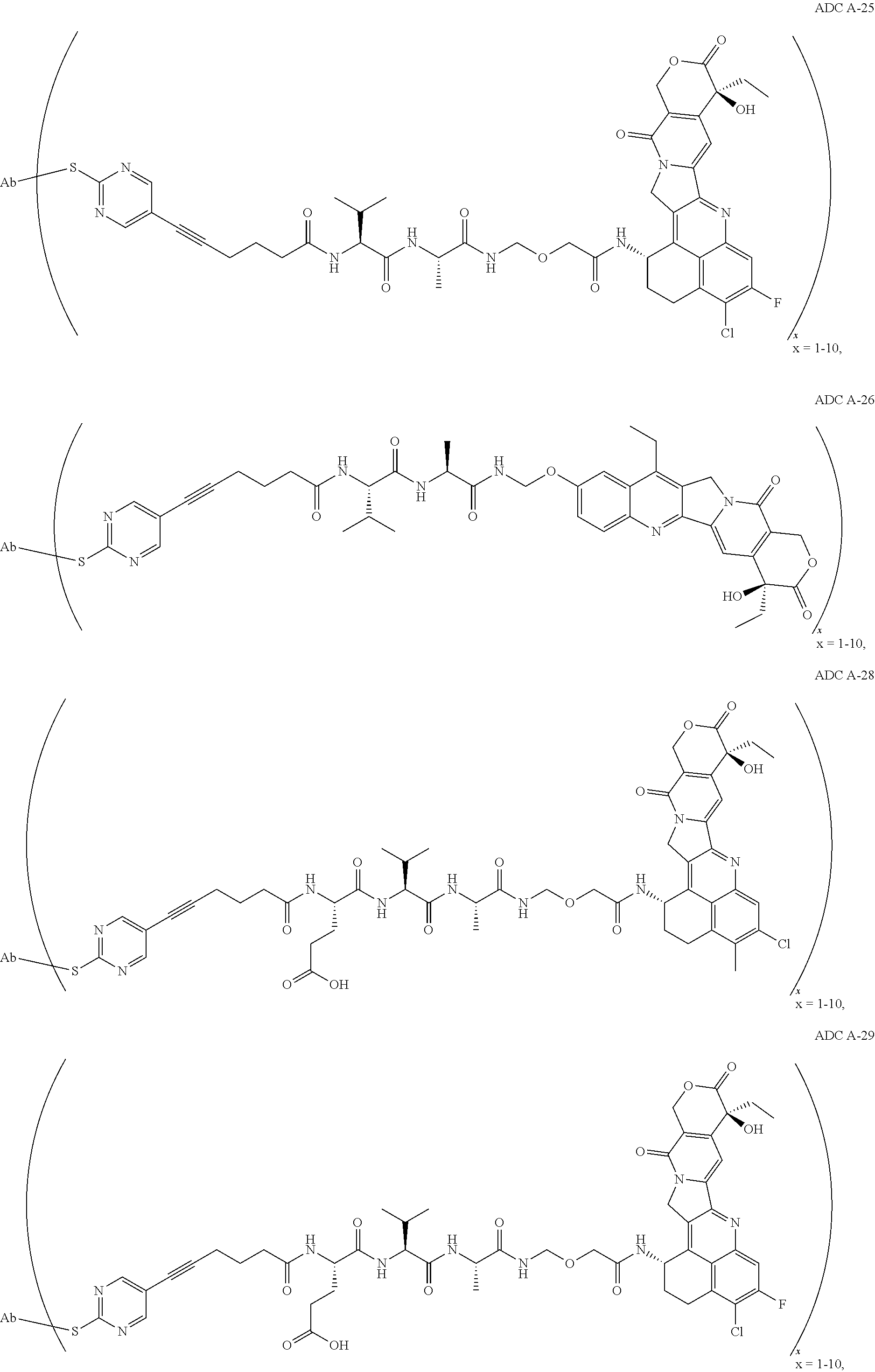

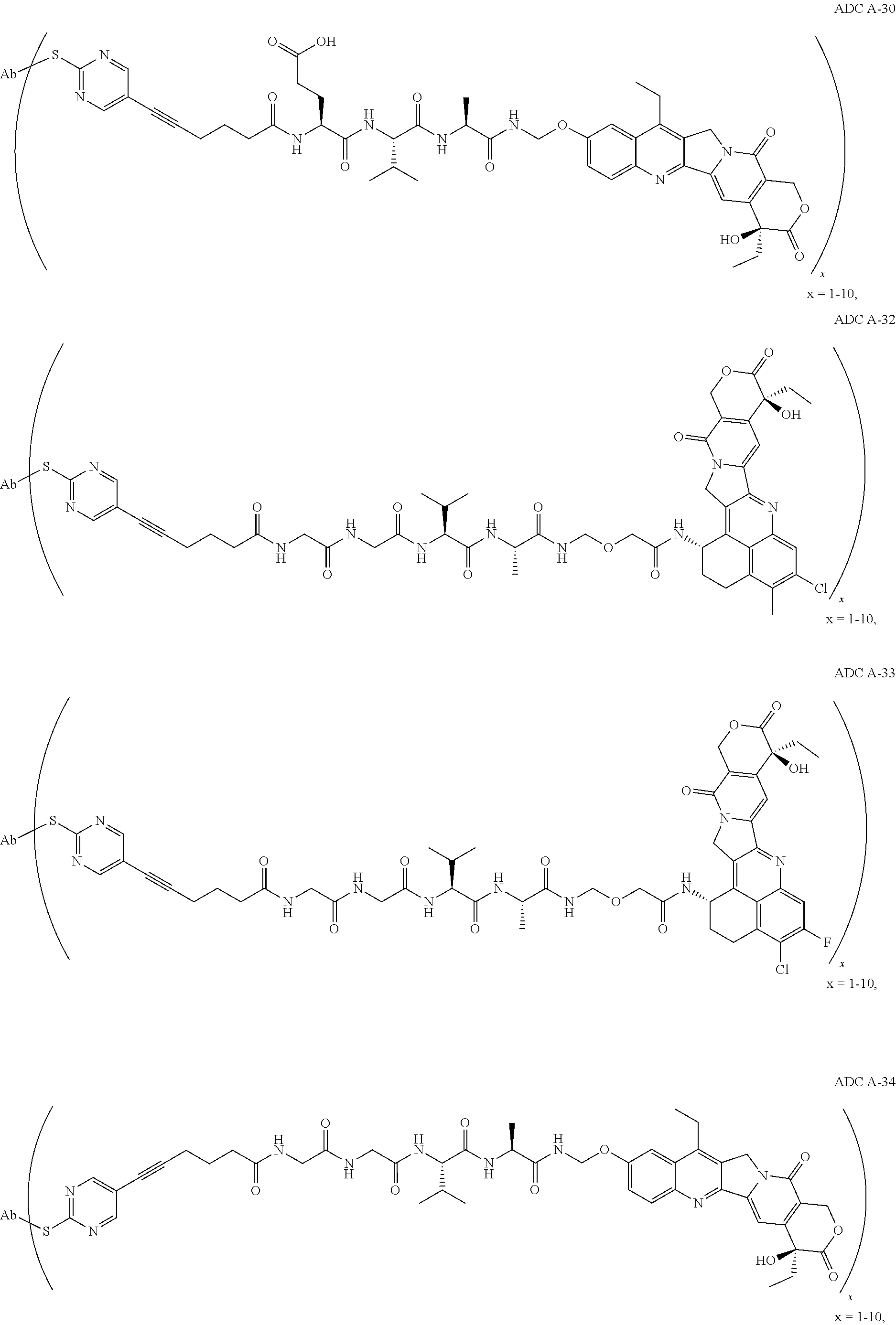

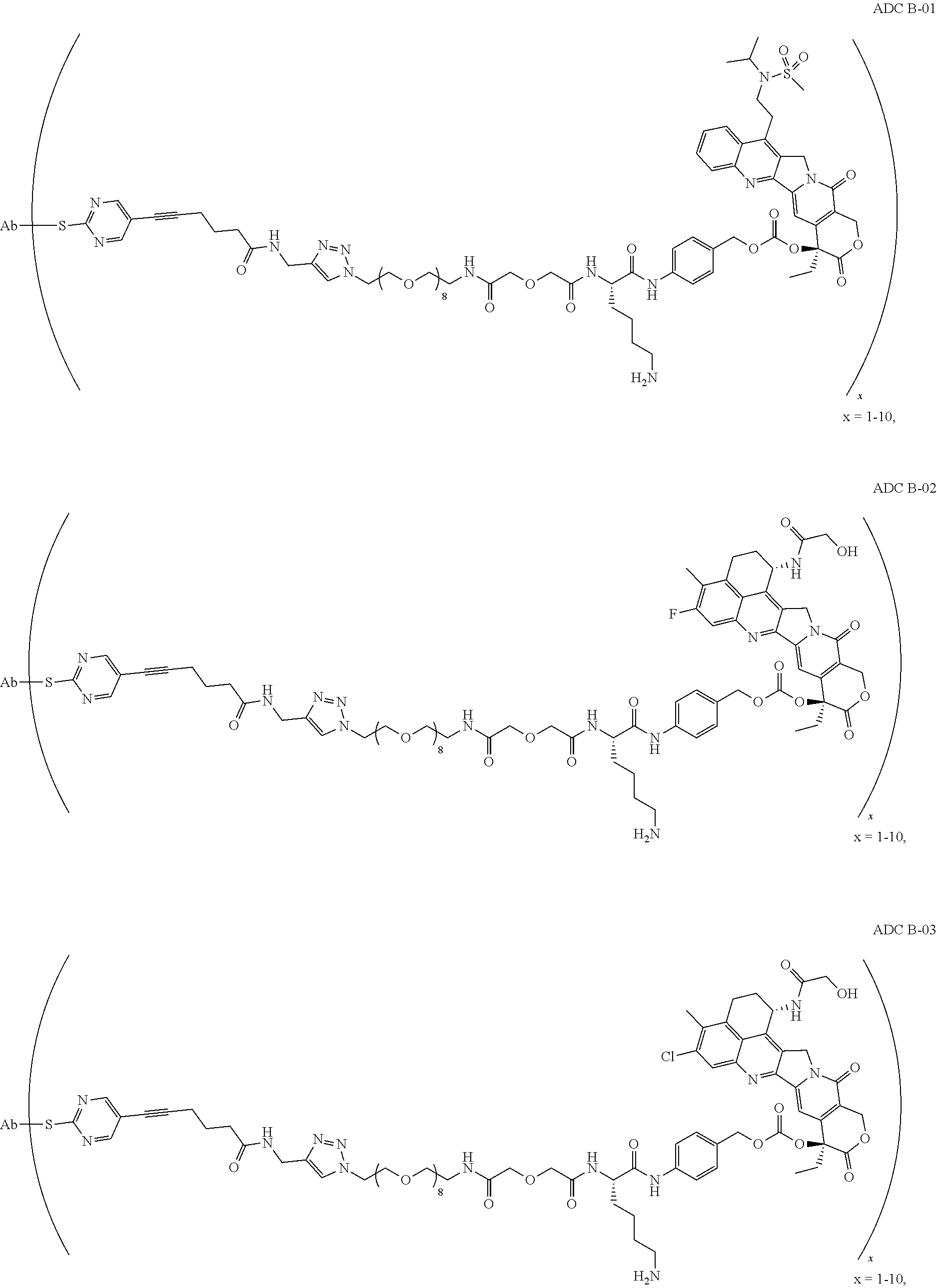

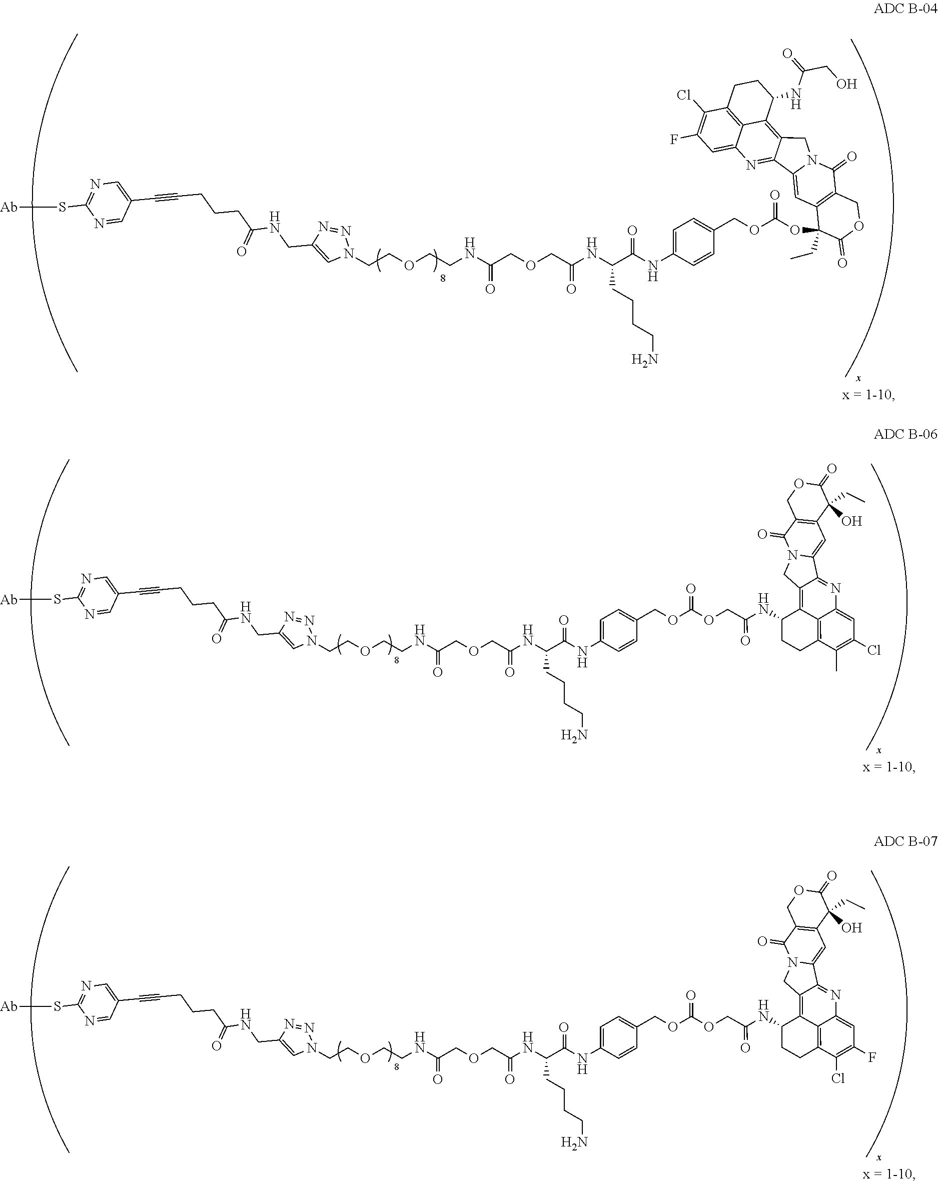

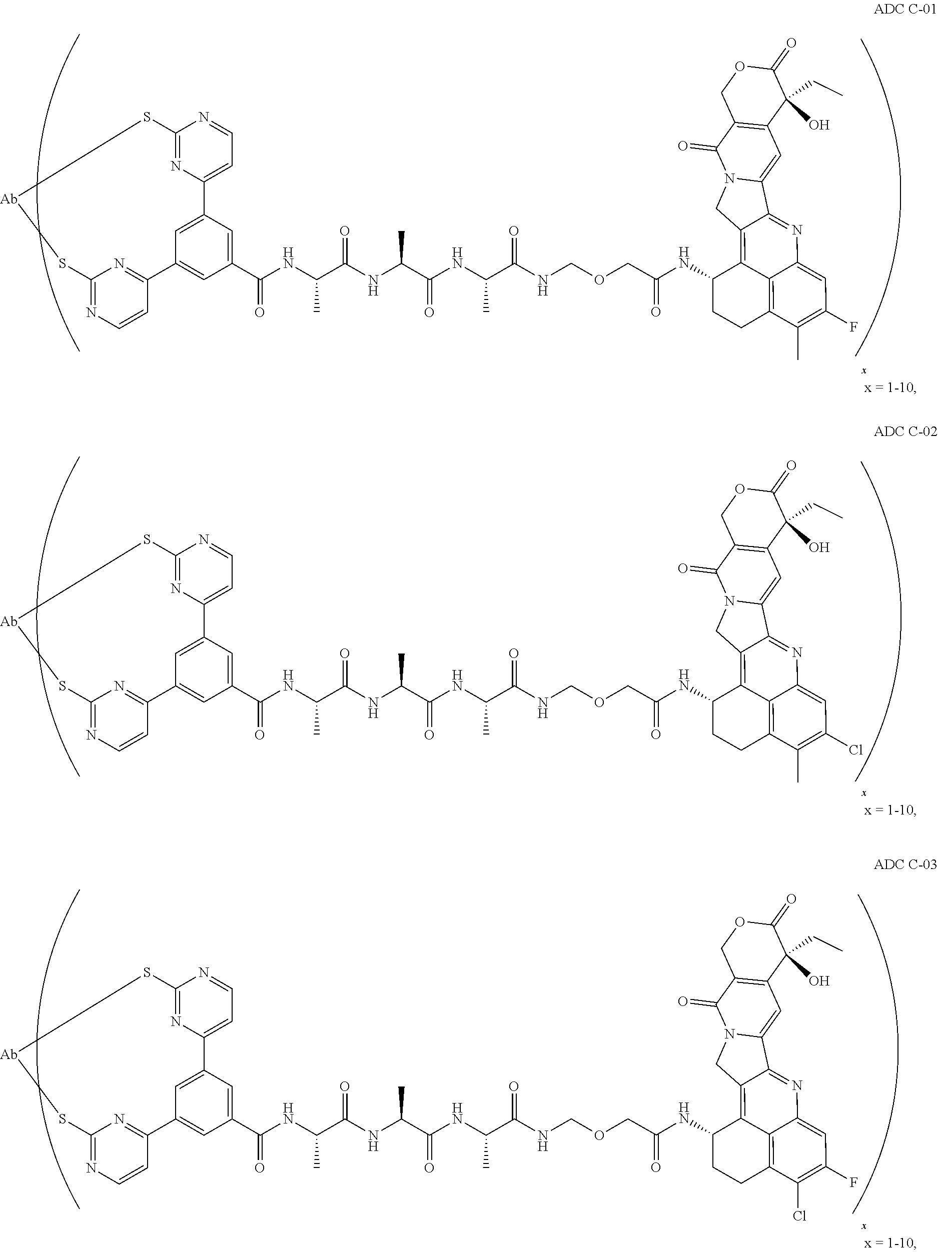

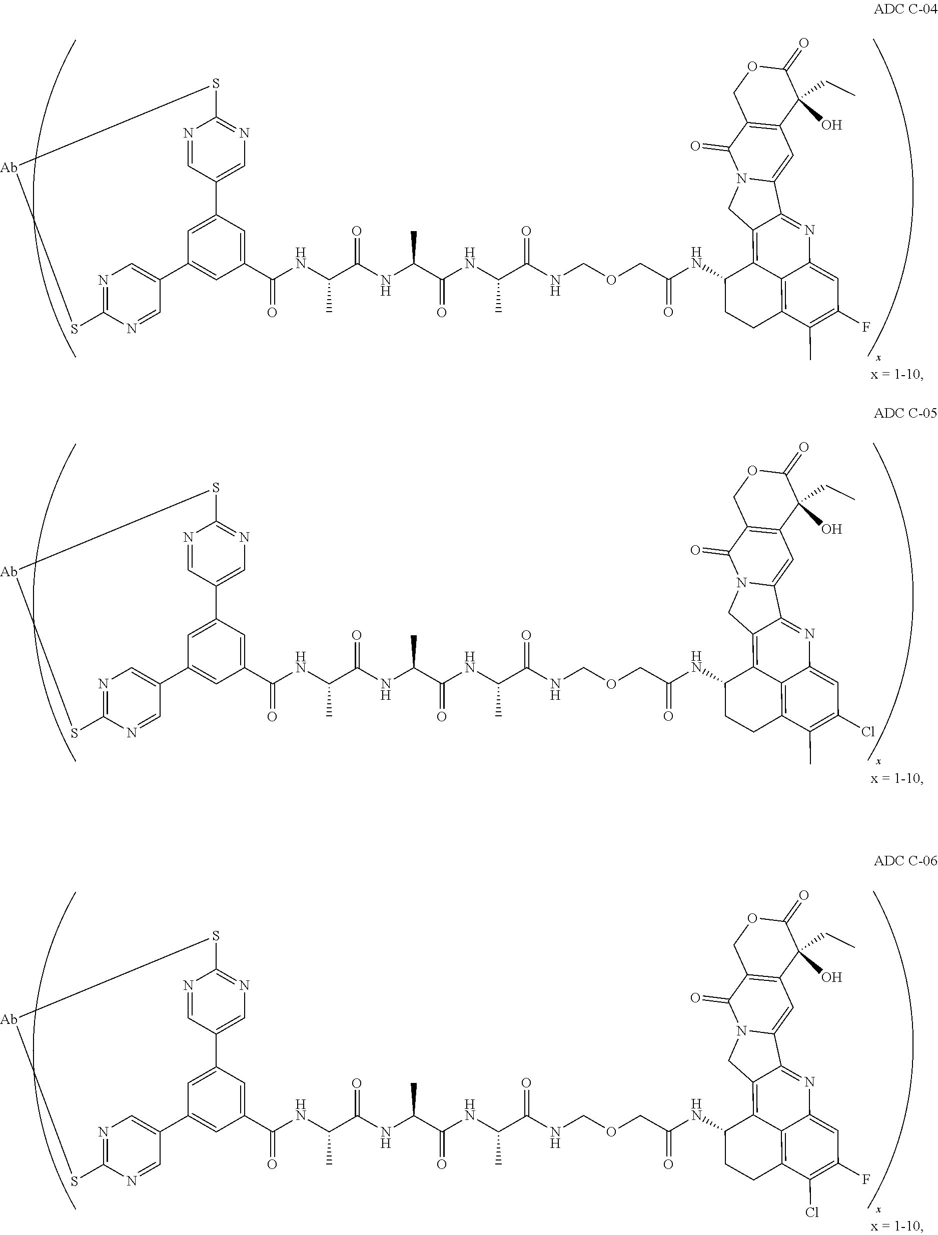

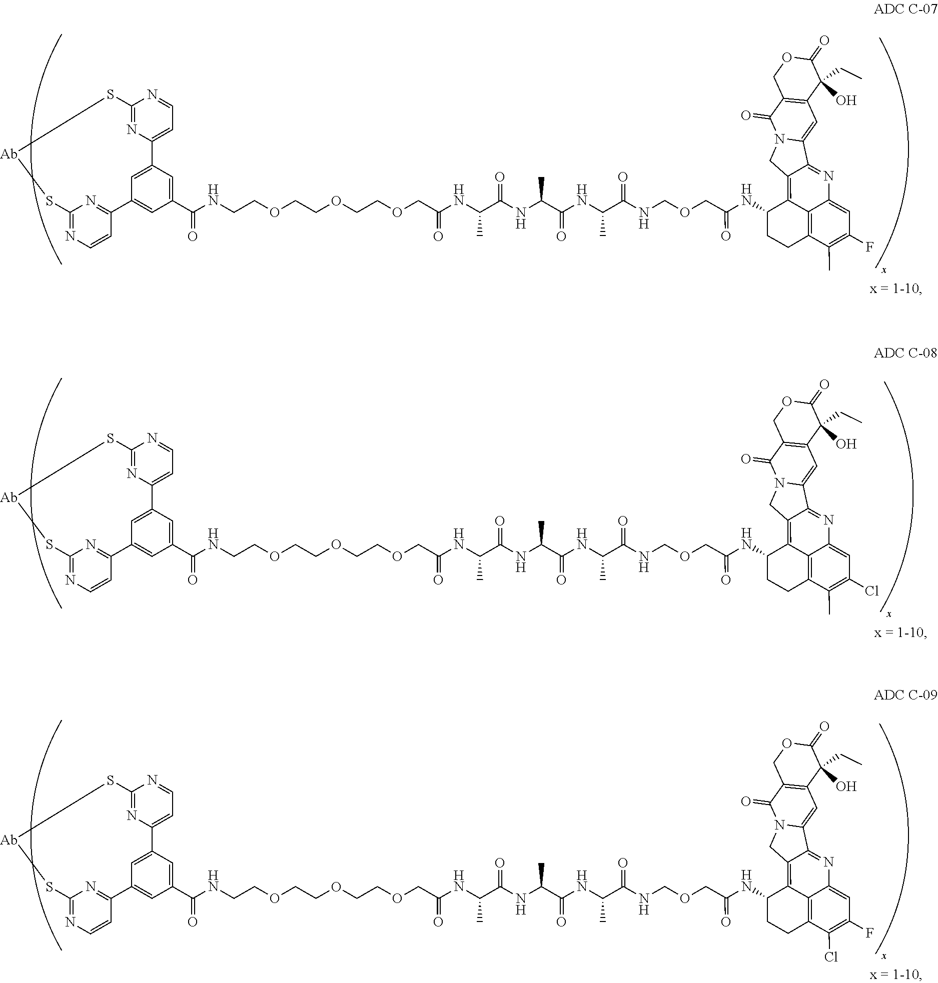

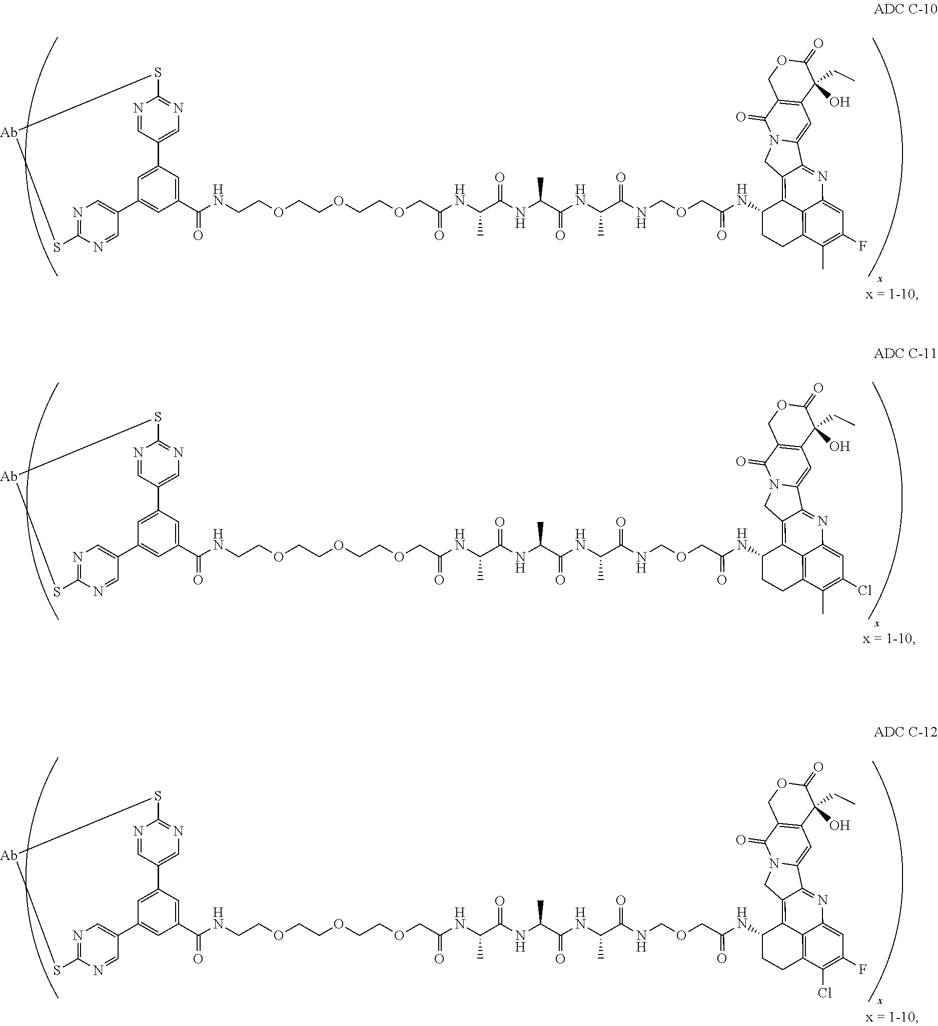

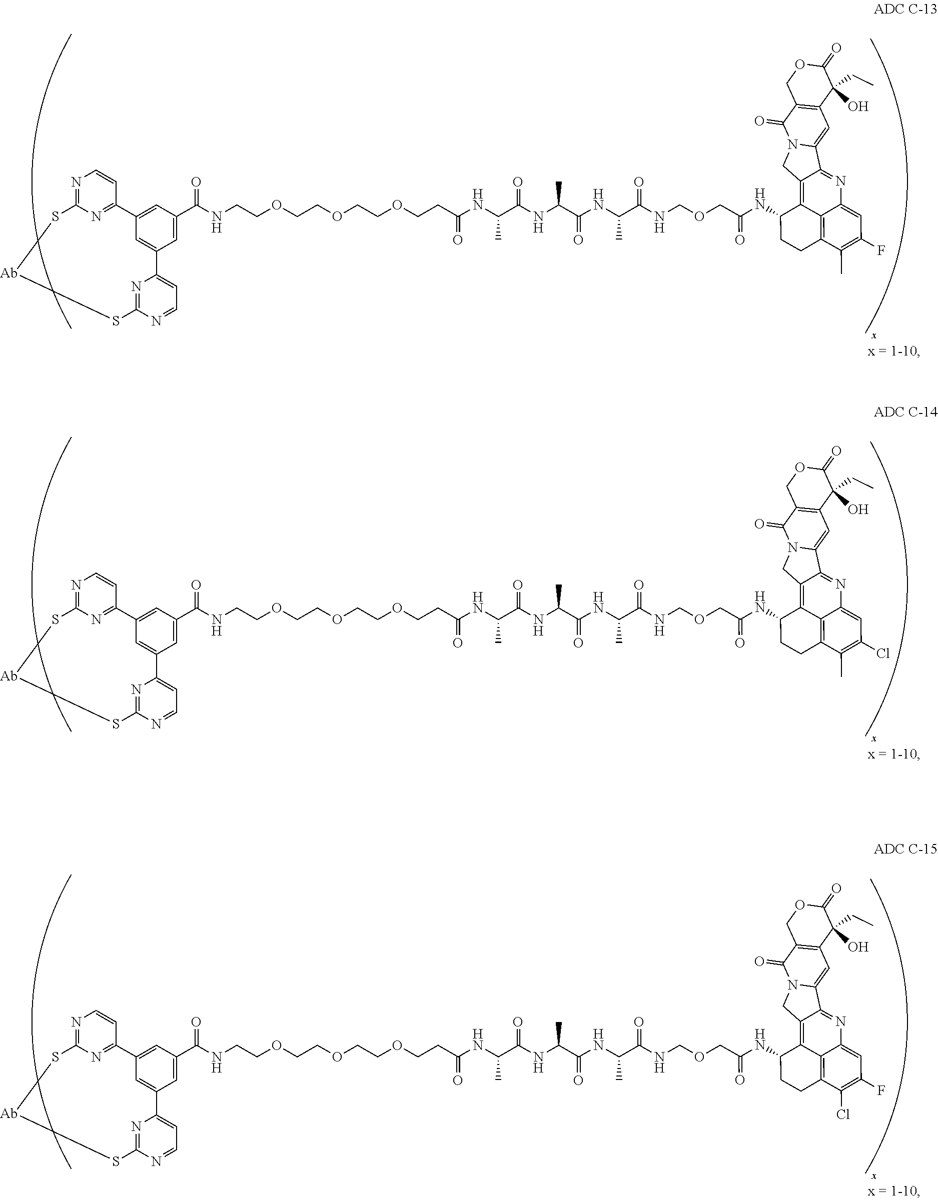

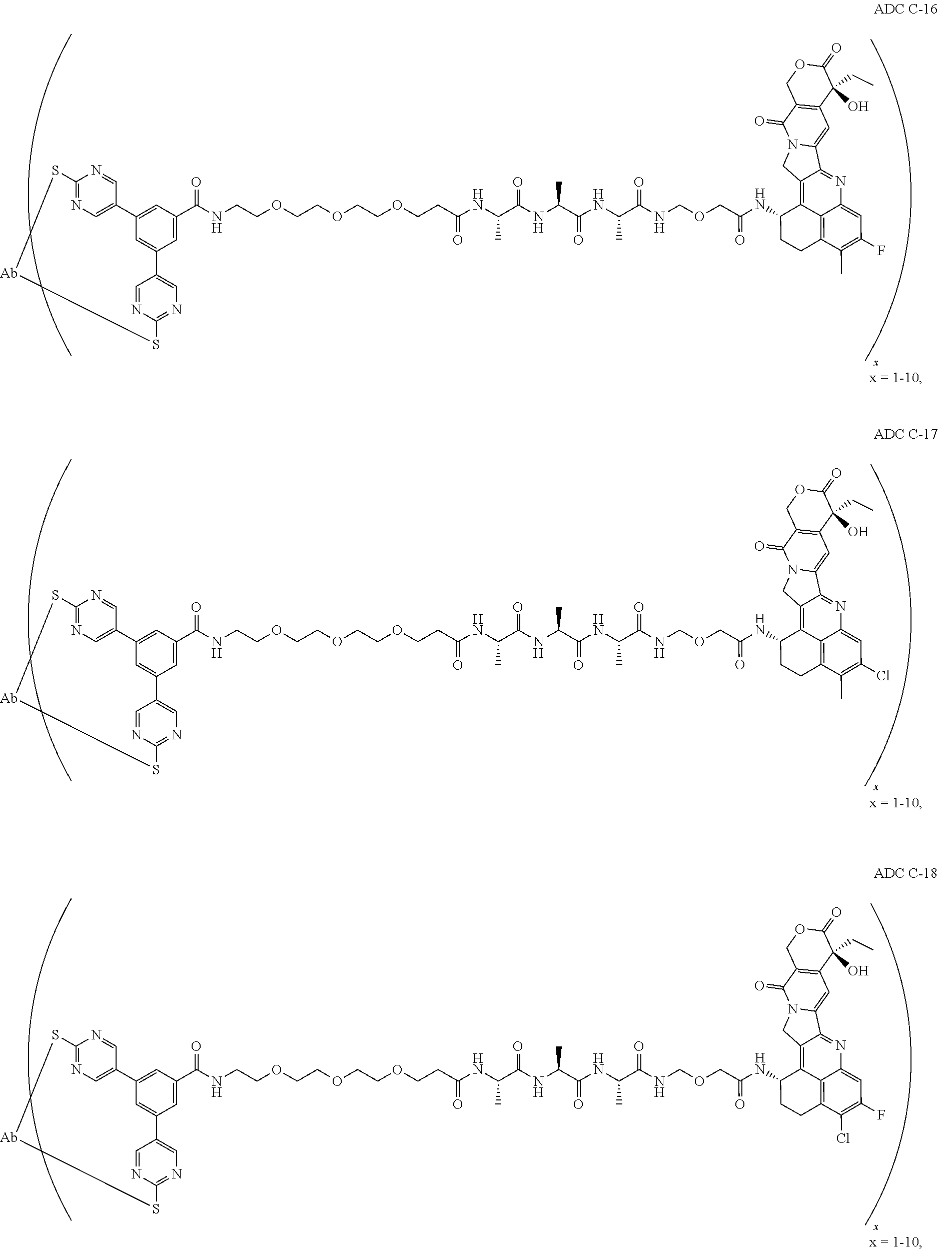

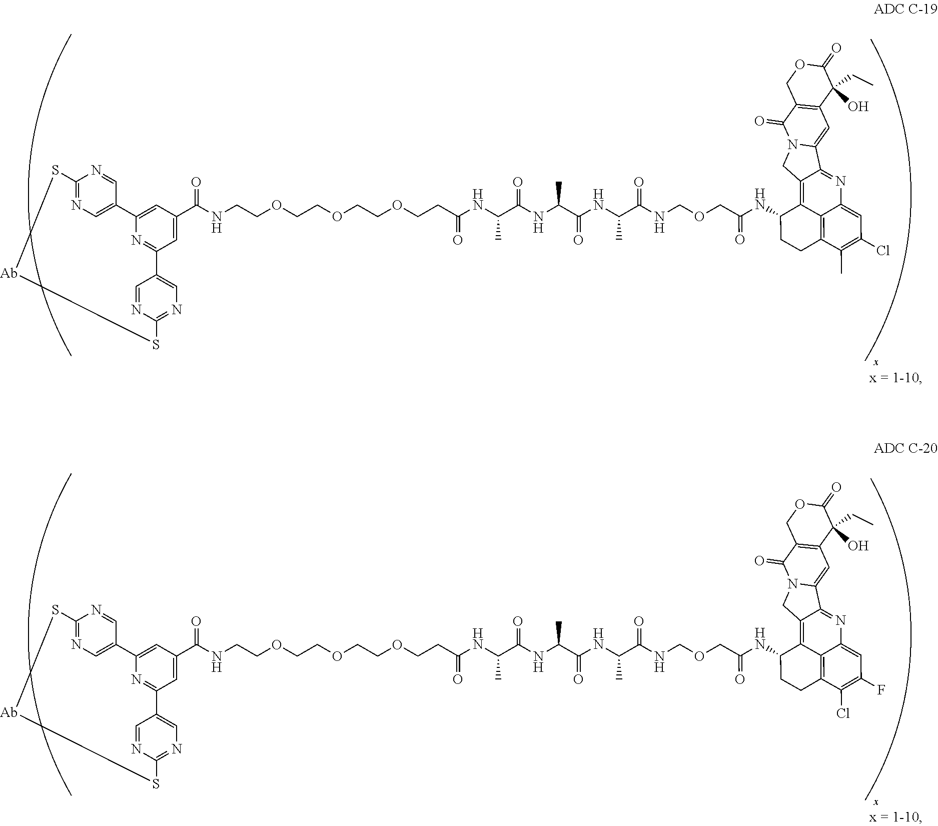

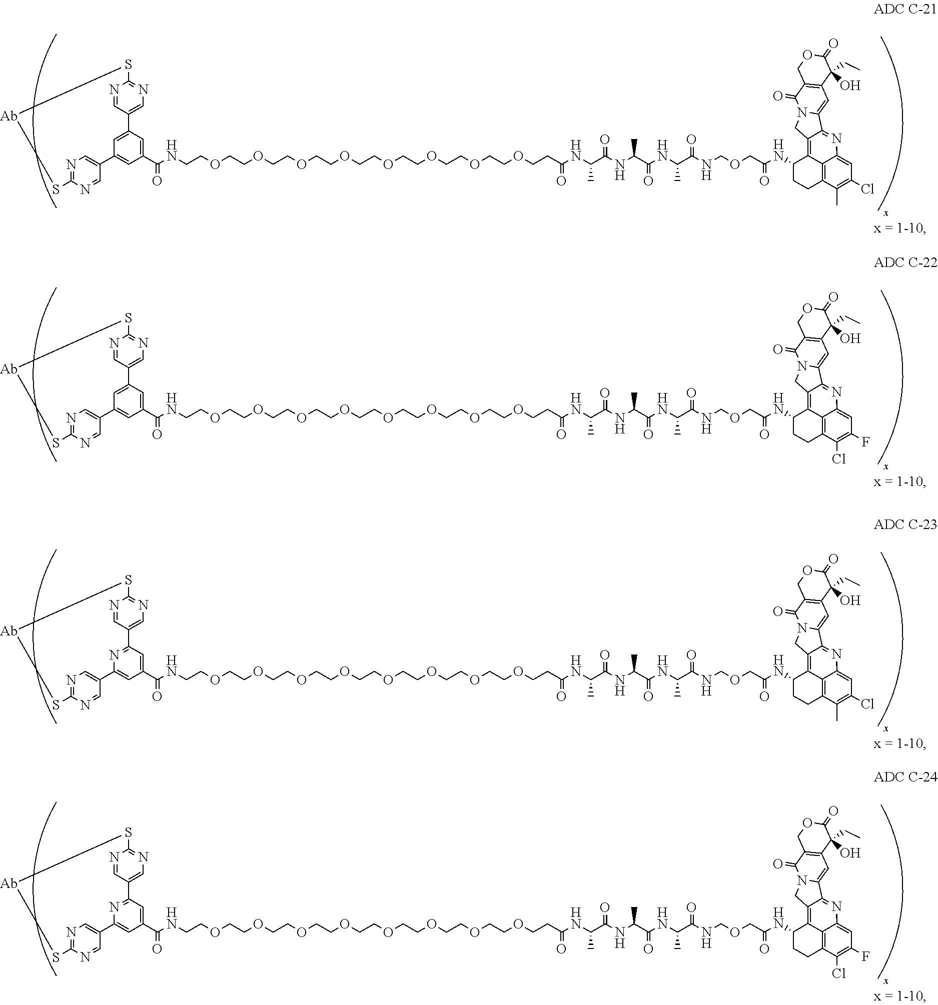

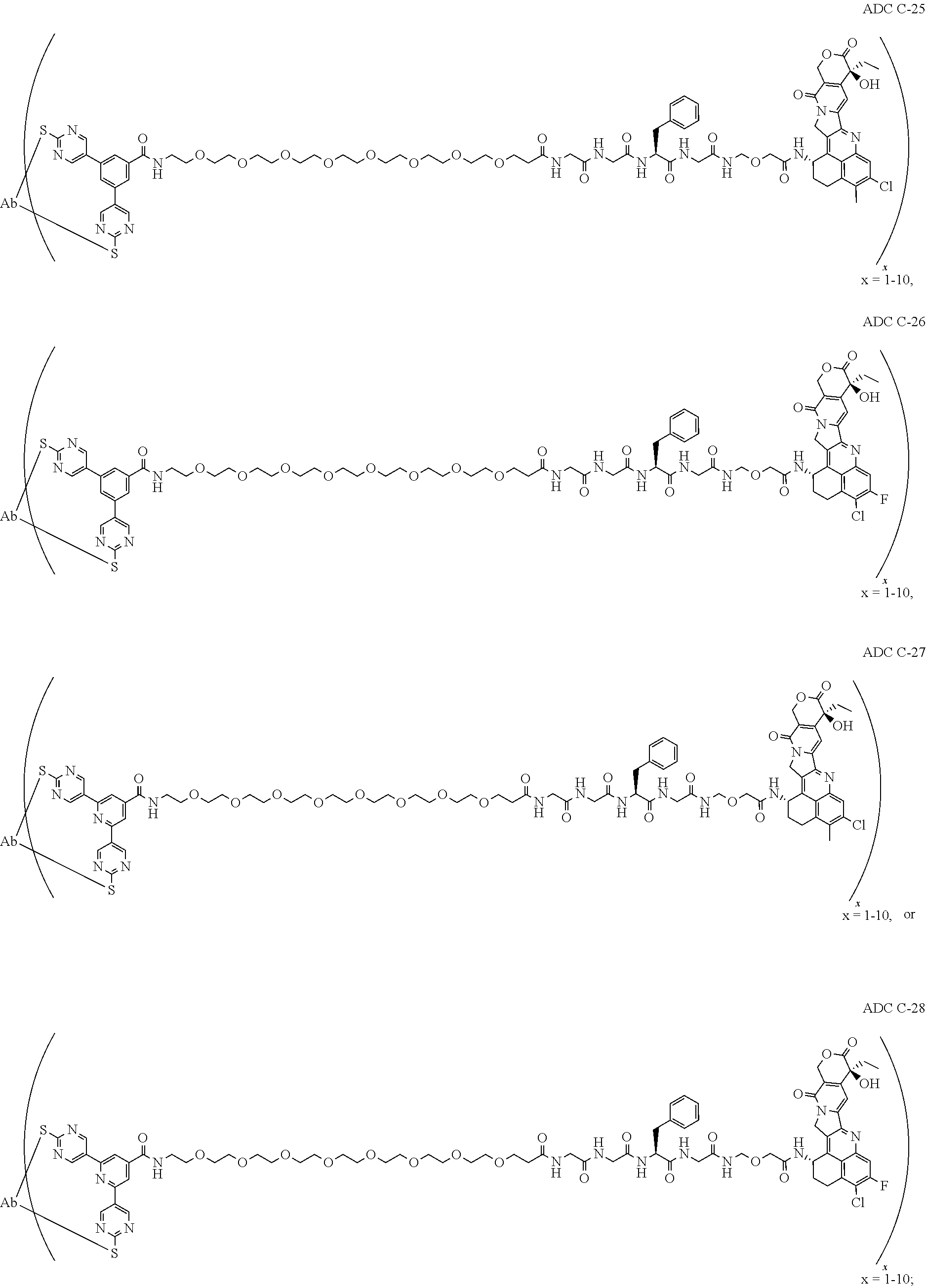

wherein Ab is a bispecific antibody or an antigen-binding fragment thereof as described herein;

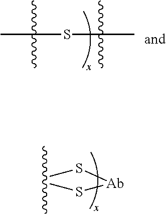

represent the specific connection method between the sulfhydryl group in the bispecific antibody or its antigen-binding fragment and other parts in the antibody-drug conjugate; and x represents the drug load quantity.

In some embodiments, the thiol group in the bispecific antibody or antigen-binding fragment thereof forms a thioether bond through an addition reaction or a substitution reaction with the precursor of other parts of the antibody-drug conjugate to obtain the antibody-drug conjugates.

In some embodiments, the Ab comprises:
(1) a peptide chain I-A comprising the amino acid sequence shown in SEQ ID NO: 1, a peptide chain I-B comprising the amino acid sequence shown in SEQ ID NO: 2 or 9, and/or a peptide chain I-C comprising the amino acid sequence shown in SEQ ID NO: 3 or 10;
(2) a peptide chain II-A comprising the amino acid sequence shown in SEQ ID NO: 4, a peptide chain II-B comprising the amino acid sequence shown in SEQ ID NO: 5 or 7, and/or a peptide chain II-C comprising the amino acid sequence shown in SEQ ID NO: 6 or 8;
(3) a peptide chain IV-A comprising the amino acid sequence shown in SEQ ID NO: 4, a peptide chain IV-B comprising the amino acid sequence shown in SEQ ID NO: 7, and/or a peptide chain IV-C comprising the amino acid sequence shown in SEQ ID NO: 11;
(4) a peptide chain V-A comprising the amino acid sequence shown in SEQ ID NO: 1, a peptide chain V-B comprising the amino acid sequence shown in SEQ ID NO: 9, a peptide chain V-C comprising the amino acid sequence shown in SEQ ID NO: 13, and/or a peptide chain V-D comprising the amino acid sequence shown in SEQ ID NO: 12; or
(5) a peptide chain VII-A comprising the amino acid sequence shown in SEQ ID NO: 14 and/or a peptide chain VII-B comprising the amino acid sequence shown SEQ ID NO: 8.

In some embodiments, the Ab comprises:
(1) a peptide chain I-A comprising the amino acid sequence shown in SEQ ID NO: 1, a peptide chain I-B comprising the amino acid sequence shown in SEQ ID NO: 2, and a peptide chain I-C peptide comprising the amino acid sequence shown in SEQ ID NO: 3;
(2) a peptide chain I-A comprising the amino acid sequence shown in SEQ ID NO: 1, a peptide chain I-B comprising the amino acid sequence shown in SEQ ID NO: 9, and a peptide chain I-C comprising the amino acid sequence shown in SEQ ID NO: 10;
(3) a peptide chain II-A comprising the amino acid sequence shown in SEQ ID NO: 4, a peptide chain II-B comprising the amino acid sequence shown in SEQ ID NO: 5, and a peptide chain II-C comprising the amino acid sequence shown in SEQ ID NO: 6;
(4) a peptide chain II-A comprising the amino acid sequence shown in SEQ ID NO: 4, a peptide chain II-B comprising the amino acid sequence shown in SEQ ID NO:7, and a peptide chain II-C comprising the amino acid sequence shown in SEQ ID NO: 8;
(5) a peptide chain IV-A comprising the amino acid sequence shown in SEQ ID NO: 4, a peptide chain IV-B comprising the amino acid sequence shown in SEQ ID NO: 7, and a peptide chain IV-C comprising the amino acid sequence shown in SEQ ID NO: 11;
(6) a peptide chain V-A comprising the amino acid sequence shown in SEQ ID NO: 1, a peptide chain V-B comprising the amino acid sequence shown in SEQ ID NO: 9, a peptide chain V-C comprising the amino acid sequence shown in SEQ ID NO: 13, and a peptide chain V-D comprising the amino acid sequence shown in SEQ ID NO:12; or
(7) a peptide chain VII-A comprising the amino acid sequence shown in SEQ ID NO: 14 and a peptide chain VII-B comprising the amino acid sequence shown in SEQ ID NO: 8.

In specific embodiments, the Ab is selected from the group consisting of BsAb 07B, BsAb 10B, BsAb 38B, BsAb 41B, BsAb 49B, BsAb 55B, and BsAb 56B.

In specific embodiments of the ADCs provided herein, a heavy chain constant region of the bispecific antibody of the ADC may comprise a C-terminal lysine, lack a C-terminal lysine, or lack a C-terminal glycine-lysine dipeptide. In some embodiments, the N-terminal amino acid of a variable region of the bispecific antibody of an ADC provided herein may comprise a glutamate or glutamine residue or a glutamate or glutamine residue cyclized to pyroglutamic acid.

In some embodiments, the N-terminal amino acid of a variable region of the bispecific antibody or antigen-binding fragment thereof of an ADC provided herein may be cyclized to pyroglutamic acid. Thus, a composition of ADCs disclosed herein may comprise a population of ADCs, wherein each ADC may independently comprise a C-terminal lysine, lack a C-terminal lysine, lack a C-terminal glycine-lysine dipeptide, and/or comprise an N-terminal glutamine or glutamate residue or an N-terminal glutamine or glutamate cyclized to pyroglutamate.

Accordingly, in some embodiments, the application further provides compositions comprising an ADC as described herein, wherein the predominant species of ADC in the composition comprises (i) an antibody wherein the heavy chain C-terminus lacks a lysine residue; (ii) an antibody in which the N-terminus of the heavy chain comprises a glutamine, glutamate, or pyroglutamate residue; (iii) an antibody in which the C-terminus of the heavy chain lacks the amino acid residue lysine and the N-terminus of the heavy chain comprises a glutamine, glutamate or pyroglutamate residue; (iv) an antibody in which the C-terminus of the heavy chain lacks the amino acid residue lysine and the N-terminus of the heavy chain comprises a pyroglutamate residue; or (v) an antibody in which the C-terminus of the heavy chain lacks the amino acid residue lysine and the N-terminus of the heavy chain comprises a glutamine or glutamate residue.

As is known to those skilled in the art, pyroglutamic acid is the conjugate acid of pyroglutamate and is in equilibrium with pyroglutamate in solution. Therefore, the present application also provides a composition comprising one or more ADCs described in any of the preceding embodiments. For example, the present invention provides ADC embodiments comprising an amino acid sequence set forth in SEQ ID NO: 2, 9, 18, or 60 wherein the N-terminal glutamate has been converted to pyroglutamate and/or embodiments comprising an amino acid sequence set forth in SEQ ID NO: 5, 7, 13, or 16 wherein the N-terminal glutamine has been converted to pyroglutamate.

In some embodiments, the composition has a DAR value (Drug-Antibody Conjugation Ratio) of 1-10, such as: 1, 2, 3, 4, 5, 6, 7, 8, 9, 10, 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10.

In some embodiments, the composition has a DAR value of 3 to 9 or of 4 to 8, for example, 3.0 to 3.5, 3.0 to 4.0, 3.0 to 4.5, 3.0 to 5.0, 3.0 to 5.5, 3.0 to 6.0, 3.5 to 4.0, 3.5 to 4.5, 3.5 to 5.0, 3.5 to 5.5, 3.5 to 6.0, 3.5 to 6.5, 3.5 to 7.0, 3.5 to 7.5, 3.5 to 8.0, 4.0 to 4.5, 4.0 to 5.0, 4.0 to 5.5, 4.0 to 6.0, 4.0 to 6.5, 4.0 to 7.0, 4.0 to 7.5, 4.0 to 8.0, 4.5 to 5.0, 4.5 to 5.5, 4.5 to 6.0, 4.5 to 6.5, 4.5 to 7.0, 4.5 to 7.5, 4.5 to 8.0, 5.0 to 5.5, 5.0 to 6.0, 5.0 to 6.5, 5.0 to 7.0, 5.0 to 7.5, 5.0 to 8.0, 5.5 to 6.0, 5.5 to 6.5, 5.5 to 7.0, 5.5 to 7.5, 5.5 to 8.0, 6.0 to 6.5, 6.0 to 7.0, 6.0 to 7.5, 6.0 to 8.5, 6.5 to 7.0, 6.5 to 7.5, 6.5 to 8.5, 7.0 to 7.5, 7.0 to 9.0 or 7.5 to 9.0.

### Conjugation

In another aspect, the present application provides a method for conjugating the linker-payloads described herein to the antibodies described herein to make the antibody-drug conjugates (ADCs) described herein.

In certain embodiments, the antibody described herein is conjugated to a linker-payload described herein via conjugation to a lysine in the antibody.

In certain embodiments, the antibody described herein is conjugated to a linker-payload described herein via conjugation to a cysteine residue in the antibody. In certain embodiments, the cysteine residues are from reduced intrachain disulfide bonds in the antibody. In certain embodiments, the cysteine residues are from reduced interchain disulfide bonds in the antibody.

In some embodiments, the antibody is conjugated to a linker-payload via conjugation to reduced interchain disulfide bonds in the antibody. For example, an IgG1 antibody consists of four polypeptide chains, two heavy chains comprising VH, CH1 and Fc (e.g., hinge, CH2 and CH3) domains, and two light chains comprising VL and CL domains, connected by interchain cysteine disulfide (—S—S—) bonds (e.g., two heavy chain-light chain interchain disulfide bonds and two hinge heavy chain-heavy chain interchain disulfide bonds). In certain embodiments, when these disulfide bonds are broken under reducing conditions, eight (8) reactive cysteine sulfhydryl moieties are produced. In certain embodiments, each of the eight reactive cysteine sulfhydryl moieties is a point of attachment for a linker-drug, such that a maximum of eight (x=8) linker-payloads may be attached to the reduced antibody. In certain embodiments, any one of the four disulfide bonds is broken under reducing conditions, two (2) reactive cysteine sulfhydryl moieties are produced. In further embodiments, each of the two reactive cysteine sulfhydryl moieties is a point of attachment for a linker-drug, such that two (x=2) linker-drugs may be attached to the reduced antibody. In certain embodiments, any two of the four disulfide bonds are broken under reducing conditions, four (4) reactive cysteine sulfhydryl moieties are produced. In further embodiments, each of the four reactive cysteine sulfhydryl moieties is a point of attachment for a linker-drug, such that four (x=4) linker-drugs may be attached to the reduced antibody. In certain embodiments, any three of the four disulfide bonds are broken under reducing conditions, six (6) reactive cysteine sulfhydryl moieties are produced. In further embodiments, each of the six reactive cysteine sulfhydryl moieties is a point of attachment for a linker-drug, such that six (x=6) linker-drugs may be attached to the reduced antibody.

In some embodiments, the interchain disulfide bond is between two cysteine residues, which are broken under reducing conditions, resulting in two reactive cysteine sulfhydryl moieties. In further embodiments, the interchain disulfide bridge in the antibody is between a heavy chain and a light chain, such as between C220 of a heavy chain according to EU numbering and C214 of a kappa light chain according to the EU and/or Kabat numbering, or between C220 of a heavy chain according to the EU numbering and C214 of a lambda light chain according to the EU and/or Kabat numbering. Additionally, or alternatively, the interchain disulfide bridge in the antibody is between two heavy chains, such as between C226 and/or C229 of a first heavy chain and C226 and/or C229 of a second heavy chain according to the EU numbering. In some embodiments, the cysteine residues are in the hinge region of the antibody. In some embodiments, the cysteine residue is at any one or more of positions 220, 226, or 229 in the heavy chain according to EU numbering (also referred to herein as C220, C226 or C229, respectively). In some embodiments, the cysteine residue is at position 214 in the light chain according to EU and/or Kabat numbering (also referred to herein as C214, such as position 214 in the kappa light chain according to EU and Kabat numbering or position 214 in the lambda light chain according to the EU and Kabat numbering). In one embodiment, the cysteine residues are at each of positions 220, 226, and 229 in the heavy chain according to the EU numbering and position 214 in the light chain, according to EU or Kabat numbering. In one embodiment, the cysteine residues are at each of positions 220, 226, and 229 in the heavy chain according to the EU numbering and position 214 in the kappa light chain, according to EU and Kabat numbering. In one embodiment, the cysteine residues are at each of positions 220, 226, and 229 in the heavy chain according to the EU numbering and position 214 in the lambda light chain, according to EU and Kabat numbering. In one embodiment, the cysteine residues are at any one or more of the following positions:
(i) any one, or any two, or any three of positions 220, 226, and 229 in a first heavy chain according to the EU numbering;
(ii) any one, or any two, or any three of positions 220, 226, and 229 in a second heavy chain according to the EU numbering;
(iii) position 214 in a first light chain according to the Kabat numbering and/or EU numbering; and/or
(iv) position 214 in a second light chain according to the Kabat numbering and/or EU numbering.

As used herein, C220, C226, and C229 refer to amino acid residues (cysteine, Cys, C) of an immunoglobulin identified according to the EU numbering. As it would be understood by one of skill in the art, such numberings accordingly represent amino acid residues of a polypeptide aligned to those identified in an immunoglobulin, such as the one shown in www.imgt.org/IMGTScientificChart/Numbering/Hu_IGHGnber.html.

As used herein, the cysteine residue at position 214 of kappa light chain refer to amino acid residues (cysteine, Cys, C) of an immunoglobulin identified according to the Eu or Kabat numbering. As it would be understood by one of skill in the art, such numberings accordingly represent amino acid residues of a polypeptide aligned to those identified in an immunoglobulin, such as the one shown in www.imgt.org/IMGTScientificChart/Numbering/Hu_IGKCnber.html.

As used herein, the cysteine residue at position 214 of lambda light chain refer to amino acid residues (cysteine, Cys, C) of an immunoglobulin identified according to the Eu or Kabat numbering. As it would be understood by one of skill in the art, such numberings accordingly represent amino acid residues of a polypeptide aligned to those identified in an immunoglobulin, such as the one shown in www.imgt.org/IMGTScientificChart/Numbering/Hu_IGLCnber.html.

In certain embodiments, the antibodies described herein comprise four interchain disulfide bonds in the hinge region which may be reduced, thereby breaking the bond, and revealing a reactive sulfhydryl moiety that may be conjugated with a maleimide moiety on a linker-payload, such as the maleimide moiety on linker-payloads described herein.

In certain embodiments, the antibodies described herein comprise lysine residues wherein the reactive amine side chains of lysine residues may be conjugated with a linker-payload, such as a maleimide moiety on linker-payloads described herein.

In one embodiment, the present disclosure provides a method of making an ADC described herein, comprising the steps of: a) providing a solution comprising the antibody; b) contacting the solution of a) with a reducing agent; c) contacting the solution of b) with a solution comprising a linker-payload or a salt thereof, as described herein, to make the ADC.

In one embodiment, the reducing agent is tris(2 carboxyethyl)phosphine (TCEP).

### Pharmaceutical Compositions

In another aspect, the present application provides a pharmaceutical composition comprising one or more ADCs or compositions as described herein and a pharmaceutically acceptable carrier and/or excipient.

In some embodiments, the pharmaceutical composition further comprises an additional pharmaceutically active agent. In certain embodiments, the additional pharmaceutically active agent is a drug having antineoplastic activity. In some embodiments, the additional pharmaceutically active agent is selected from: EGFR inhibitors, HER2 inhibitors, HER3 inhibitors, HER4 inhibitors, IGFR-1 inhibitors, mTOR inhibitors, PI3 kinase inhibitors, c-MET or VEGF inhibitors, chemotherapy drugs, and any combination thereof. In certain embodiments, the ADC or composition as described herein and the additional pharmaceutically active agent are provided as separate components or as mixed components.

In certain embodiments, the ADC or composition in the pharmaceutical composition of the invention is sufficient (e.g., in a subject) to exert a tumor suppressive effect (e.g., a tumor suppressive effect superior to a monospecific anti-c-MET antibody and/or a monospecific anti-EGFR antibody, wherein the amino acid sequences of the CDRs of the monospecific anti-c-MET antibody are identical to the amino acid sequences of the CDRs of the first antigen-binding domain, and the amino acid sequences of the CDRs of the monospecific anti-EGFR antibody are identical to the amino acid sequences of the CDRs of the second antigen-binding domain).

In certain embodiments, the tumor suppressive effects include: inhibition of EGFR and c-MET signaling, antibody-dependent cell-mediated cytotoxicity (ADCC) activity, and/or complement-dependent cytotoxicity (CDC) activity.

In some embodiments, the ADCs provided herein are formulated in unit injection form with a pharmaceutically acceptable parenteral carrier for parenteral use, such as intravenous bolus injection, intravenous injection, intratumoral injection, and the like. Optionally, the ADC with the desired purity is mixed with a pharmaceutically acceptable diluent, carrier, excipient or stabilizer to prepare a lyophilized or solution form (Remington's Pharmaceutical Sciences (1980) p. 16th edition, Osol, A. Ed.). The ADCs described herein or pharmaceutical compositions comprising the ADCs may be administered to a subject by any suitable route.

### Therapeutic Uses of the ADCs

In another aspect, provided herein is the use of any of the ADCs, compositions, or pharmaceutical compositions disclosed herein in preparing a medicament for prevention and/or treatment and/or in a subject or adjuvant treatment of diseases related to c-MET and/or EGFR, and/or for inhibiting the activity of c-MET and/or EGFR in vitro or in a subject; wherein the method is related to c-MET- and/or EGFR-related diseases, including but not limited to cancers associated with EGFR activating mutations, EGFR gene amplification, elevated circulating HGF levels, c-MET activating mutations, and/or c-MET gene amplification.

In some embodiments, the cancer is selected from epithelial cell carcinoma, breast cancer, ovarian cancer, lung adenocarcinoma, small cell lung cancer, non-small cell lung cancer, oral cancer, colorectal cancer, anal cancer, prostate cancer, bladder cancer, pharyngeal cancer, nasal cancer, pancreatic cancer, skin cancer, tongue cancer, esophageal cancer, vaginal cancer, cervical cancer, spleen cancer, testicular cancer, gastric cancer, thymus cancer, thyroid cancer, hepatocellular carcinoma, sporadic or hereditary papillary renal cell carcinoma, colon cancer, liver cancer, kidney cancer, or head and neck cancer.

In some embodiments, the ADC or pharmaceutical composition is administered in combination with an additional pharmaceutically active agent, e.g., administered simultaneously, separately or sequentially. In certain embodiments, the additional pharmaceutically active agent is a drug having antineoplastic activity. In certain embodiments, the additional pharmaceutically active agent is selected from: EGFR inhibitors, HER2 inhibitors, HER3 inhibitors, HER4 inhibitors, IGFR-1 inhibitors, mTOR inhibitors, PI3 kinase inhibitors, c-MET or VEGF inhibitors, chemotherapy drugs, or any combination thereof.

In another aspect, the present invention provides a method of inhibiting the activity of c-MET and/or EGFR in a cell, the method comprising contacting the cell with an ADC composition or pharmaceutical composition as described herein. In certain embodiments, the cell is a cell (e.g., a tumor cell) that expresses c-MET and/or EGFR.

In another aspect, the present invention provides a method for preventing, treating, and/or acting as an adjuvant in treating a disease related to c-MET and/or EGFR in a subject, the method comprising administering an effective amount of an ADC, composition, and/or pharmaceutical composition provided herein to a subject in need thereof.

In certain embodiments, the disease related to c-MET and/or EGFR is cancer. In certain embodiments, the cancer is associated with EGFR activating mutations, EGFR gene amplification, elevated levels of circulating HGF, c-MET activating mutations and/or c-MET gene amplification.

In certain embodiments, the cancer is epithelial cell carcinoma, breast cancer, ovarian cancer, lung adenocarcinoma, small cell lung cancer, non-small cell lung cancer, oral cancer, colorectal cancer, anal cancer, prostate cancer, bladder cancer, pharyngeal cancer, nasal cancer, pancreas cancer, skin cancer, tongue cancer, esophagus cancer, vagina cancer, cervix cancer, spleen cancer, testis cancer, gastric cancer, thymus cancer, thyroid cancer, hepatocellular carcinoma, sporadic or hereditary papillary renal cell carcinoma, colon cancer, liver cancer, kidney cancer or head and neck cancer. In certain embodiments, the method further comprises administering to the subject a second therapy selected from surgery, chemotherapy, radiotherapy, immunotherapy, gene therapy, DNA therapy, RNA therapy, nanotherapy, virotherapy, adjuvant therapy, and any combination thereof.

In certain embodiments, the second therapy may be applied simultaneously, separately or sequentially to the methods described above.

In particular embodiments, the present invention provides an ADC, composition or pharmaceutical composition described herein for use in a method of preventing, treating, and/or acting as an adjuvant in treating a disease related to c-MET and/or EGFR in a subject. In certain embodiments, the disease related to c-MET and/or EGFR is cancer. In certain embodiments, the cancer is associated with EGFR activating mutations, EGFR gene amplification, elevated circulating HGF levels, c-MET activating mutations, and/or c-MET gene amplification.

In some embodiments, the cancer is selected from epithelial cell carcinoma, breast cancer, ovarian cancer, lung adenocarcinoma, small cell lung cancer, non-small cell lung cancer, oral cancer, colorectal cancer, anal cancer, prostate cancer, bladder cancer, Pharyngeal cancer, nasal cancer, pancreatic cancer, skin cancer, tongue cancer, esophageal cancer, vaginal cancer, cervical cancer, spleen cancer, testicular cancer, gastric cancer, thymus cancer, thyroid cancer, hepatocellular carcinoma, sporadic or hereditary papillary kidney cell carcinoma, colon cancer, liver cancer, kidney cancer, or head and neck cancer.

In some embodiments, the method further comprises administering to the subject a second therapy, the second therapy being selected from the group consisting of surgery, chemotherapy, radiotherapy, immunotherapy, gene therapy, DNA therapy, RNA therapy, nanotherapy, viral therapy, adjuvant therapy and any combination thereof. In certain embodiments, the second therapy can be applied simultaneously, separately or sequentially.

The ADC of the present invention and the pharmaceutical composition of the present invention can be formulated into any dosage form known in the medical field, for example, tablet, pill, suspension, emulsion, solution, gel, capsule, powder, granule, elixir, lozenge, suppository, injection (including solution for injection, sterile powder for injection and concentrated solution for injection), inhalant, spray, etc. The preferred dosage form depends on the intended mode of administration and therapeutic use. Pharmaceutical compositions of the invention should be sterile and stable under the conditions of manufacture and storage. A preferred dosage form is injection. Such injection can be a sterile solution for injection. For example, the sterile solution for injection can be prepared by incorporating in an appropriate solvent a necessary dose of the ADC or the pharmaceutical composition of the present invention, and optionally, simultaneously incorporating other desired ingredients (including but not limited to, pH adjusting agent, surfactant, adjuvant, ionic strength enhancer, isotonic agent, preservative, diluent, or any combination thereof), followed by filter sterilization. In addition, a sterile solution for injection can be prepared as sterile lyophilized powder (e.g., by vacuum drying or freeze-drying) for ease of storage and use. Such sterile lyophilized powder can be dispersed in a suitable carrier before use, such as sterile pyrogen-free water.

Furthermore, the ADCs of the present invention may be presented in pharmaceutical compositions in unit dosage form for ease of administration.

The ADC and the pharmaceutical composition of the present invention can be administered by any suitable method known in the art, including but not limited to, oral, buccal, sublingual, ocular, topical, parenteral, rectal, intrathecal, intracytoplasmic reticulum, groin, intravesical, topical (e.g., powder, ointment, or drops), or nasal route. However, for many therapeutic uses, the preferred route/mode of administration is parenteral (e.g., intravenous injection, subcutaneous injection, intraperitoneal injection, intramuscular injection). The skilled artisan will understand that the route and/or mode of administration will vary depending on the intended purpose. In some preferred embodiments, the ADC and the pharmaceutical composition of the present invention are administered by intravenous injection or injection.

The pharmaceutical composition of the present invention may comprise a “therapeutically effective amount” or “prophylactically effective amount” of the bispecific antibody of the present invention. The “prophylactically effective amount” means an amount sufficient to prevent, arrest, or delay the occurrence of a disease. The “therapeutically effective amount” refers to an amount sufficient to cure, or at least partially prevent the disease and its complications in a patient already suffering from the disease. The therapeutically effective amount of the bispecific antibody of the invention may vary according to the following factors: The severity of the disease to be treated, the general state of the patient's immune system, the general condition of the patient such as age, weight and sex, the mode of administration of the drug, and other treatments administered at the same time, etc.

In the present invention, dosing regimens can be adjusted to obtain the optimum desired response (e.g., a therapeutic or prophylactic response). For example, a single dose can be administered, multiple doses can be administered over time or the dose can be proportionally reduced or increased as indicated by the exigencies of the therapeutic situation.

In the present invention, the subject may be a mammal, such as a human.

### Kits

Also provided are kits comprising one or more ADCs described herein, or pharmaceutical compositions thereof. In a specific embodiment, provided herein is a pharmaceutical pack or kit comprising one or more containers filled with one or more of the ingredients of the pharmaceutical compositions described herein, such as one or more ADCs provided herein. In certain embodiments, the kits comprise a pharmaceutical composition described herein and any prophylactic or therapeutic agent, such as those described herein. In certain embodiments, the kits may comprise a T cell mitogen, such as, e.g., phytohaemagglutinin (PHA) and/or phorbol myristate acetate (PMA), or a TCR complex stimulating antibody, such as an anti-CD3 antibody and anti-CD28 antibody. Optionally associated with such container(s) can be a notice in the form prescribed by a governmental agency regulating the manufacture, use or sale of pharmaceuticals or biological products, which notice reflects approval by the agency of manufacture, use or sale for human administration.

Also provided are kits that can be used in the methods described herein. In certain embodiments, a kit comprises an ADC described herein, preferably purified ADC, in one or more containers. In a specific embodiment, kits described herein contain a substantially isolated c-MET and/or EGFR antigen as a control. In another specific embodiment, the kits described herein further comprise a control antibody which does not react with c-MET or EGFR antigen. In another specific embodiment, kits described herein contain one or more elements for detecting the binding of an antibody to a c-MET and/or EGFR antigen (e.g., the antibody can be conjugated to a detectable substrate such as a fluorescent compound, an enzymatic substrate, a radioactive compound or a luminescent compound, or a second antibody which recognizes the first antibody can be conjugated to a detectable substrate). In specific embodiments, a kit provided herein can include a recombinantly produced or chemically synthesized c-MET and/or EGFR antigen. The antigen provided in the kit can also be attached to a solid support. In a more specific embodiment, the detecting means of the above-described kit includes a solid support to which a c-MET and/or EGFR antigen is attached. Such a kit can also include a non-attached reporter-labeled anti-human antibody or anti-mouse/rat antibody. In this embodiment, binding of the bispecific antibody to the antigen can be detected by binding of the said reporter-labeled antibody.

### SPECIFIC EMBODIMENTS OF THE PRESENT INVENTION INCLUDE

Embodiment 1. An antibody-drug conjugate having the structure shown in the formula

Ab-[M-L-E-D]ₓ

wherein: Ab is a bispecific antibody or bispecific antigen-binding fragment thereof comprising a first antigen-binding domain that specifically binds to c-MET and a second antigen-binding domain that specifically binds to EGFR; M is a linker site connected to the bispecific antibody or bispecific antigen-binding fragment thereof, L is a structural fragment connecting the linker sites M and E; E is a structural fragment connecting L and D; D is a cytotoxic drug or residue thereof, x is selected as any integer from 1 to 10.

Embodiment 2. The antibody-drug conjugate of embodiment 1, wherein the first antigen-binding domain comprises a first light chain variable region (VL) and a first heavy chain variable region (VH), the first light chain variable region (VL) and the first heavy chain variable region (VH) together form a domain capable of specifically binding c-MET; the second antigen-binding domain includes a second light chain variable region (VL) and a second heavy chain variable region (VH), which together form a specific domain that binds EGFR; wherein, the first light chain (LC) variable region (VL) comprises:
(i) a complementarity determining region (CDR)-L1 comprising the amino acid sequence shown in SEQ ID NO: 34, a CDR-L2 comprising the amino acid sequence shown in SEQ ID NO: 36, and a CDR-L3 comprising the amino acid sequence shown in SEQ ID NO: 38, wherein the CDRs are defined by the Kabat numbering system;
(ii) a CDR-L1 comprising the amino acid sequence shown in SEQ ID NO: 34, a CDR-L2 comprising the amino acid sequence shown in SEQ ID NO: 36, and a CDR-L3 comprising the amino acid sequence shown in SEQ ID NO: 38, wherein the CDRs are defined by the Chothia numbering system;
(iii) a CDR-L1 comprising the amino acid sequence shown in SEQ ID NO: 34, a CDR-L2 comprising the amino acid sequence shown in SEQ ID NO: 36, and a CDR-L3 comprising the amino acid sequence shown in SEQ ID NO: 38, wherein the CDRs are defined by the Abm numbering system; or
(iv) a CDR-L1 comprising the amino acid sequence shown in SEQ ID NO: 35, a CDR-L2 comprising the amino acid sequence shown in SEQ ID NO: 37, and a CDR-L3 comprising the amino acid sequence shown in SEQ ID NO: 38, wherein the CDRs are defined by the IMGT numbering system;
   and/or the first heavy chain (HC) variable region (VH) contains:
(i) a CDR-H1 comprising the amino acid sequence shown in SEQ ID NO: 39, a CDR-H2 comprising the amino acid sequence shown in SEQ ID NO: 43, and a CDR-H3 comprising the amino acid sequence shown in SEQ ID NO: 47, wherein the CDRs are defined by the Kabat numbering system;
(ii) a CDR-H1 comprising the amino acid sequence shown in SEQ ID NO: 40, a CDR-H2 comprising the amino acid sequence shown in SEQ ID NO: 44, and a CDR-H3 comprising the amino acid sequence shown in SEQ ID NO: 47, wherein the CDRs are defined by the Chothia numbering system;
(iii) a CDR-H1 comprising the amino acid sequence shown in SEQ ID NO: 42, a CDR-H2 comprising the amino acid sequence shown in SEQ ID NO: 46, and a CDR-H3 comprising the amino acid sequence shown in SEQ ID NO: 47, wherein the CDRs are defined by the Abm numbering system; or
(iv) a CDR-H1 comprising the amino acid sequence shown in SEQ ID NO: 41, a CDR-H2 comprising the amino acid sequence shown in SEQ ID NO: 45, and a CDR-H3 comprising the amino acid sequence shown in SEQ ID NO: 48, where the CDRs are defined by the IMGT numbering system.

Embodiment 3. The antibody-drug conjugate of embodiment 2, wherein the second light chain (LC) variable region (VL) comprises:
(i) a complementarity determining region (CDR)-L1 comprising the amino acid sequence shown in SEQ ID NO: 19, a CDR-L2 comprising the amino acid sequence shown in SEQ ID NO: 21, and a CDR-L3 comprising the amino acid sequence shown in SEQ ID NO: 23, wherein the CDRs are defined by the Kabat numbering system;
(ii) a complementarity determining region (CDR)-L1 comprising the amino acid sequence shown in SEQ ID NO: 19, a CDR-L2 comprising the amino acid sequence shown in SEQ ID NO: 21, and a CDR-L3 comprising the amino acid sequence shown in SEQ ID NO: 23, wherein the CDRs are defined by the Chothia numbering system;
(iii) a complementarity determining region (CDR)-L1 comprising the amino acid sequence shown in SEQ ID NO: 19, a CDR-L2 comprising the amino acid sequence shown in SEQ ID NO: 21, and a CDR-L3 comprising the amino acid sequence shown in SEQ ID NO: 23, wherein the CDRs are defined by the Abm numbering system; or
(iv) a complementarity determining region (CDR)-L1 comprising the amino acid sequence shown in SEQ ID NO: 20, a CDR-L2 comprising the amino acid sequence shown in SEQ ID NO: 22, and a CDR-L3 comprising the amino acid sequence shown in SEQ ID NO: 23, wherein the CDRs are defined by the IMGT numbering system;
   and/or the second heavy chain (HC) variable region (VH) contains:
(i) a CDR-H1 comprising the amino acid sequence shown in SEQ ID NO: 24, a CDR-H2 comprising the amino acid sequence shown in SEQ ID NO: 28, and a CDR-H3 comprising the amino acid sequence shown in SEQ ID NO: 32, wherein the CDRs are defined by the Kabat numbering system;
(ii) a CDR-H1 comprising the amino acid sequence shown in SEQ ID NO: 25, a CDR-H2 comprising the amino acid sequence shown in SEQ ID NO: 29, and a CDR-H3 comprising the amino acid sequence shown in SEQ ID NO: 32, wherein the CDRs are defined by the Chothia numbering system;
(iii) a CDR-H1 comprising the amino acid sequence shown in SEQ ID NO: 27, a CDR-H2 comprising the amino acid sequence shown in SEQ ID NO: 31, and a CDR-H3 comprising the amino acid sequence shown in SEQ ID NO: 32, wherein the CDRs are defined by the Abm numbering system; or
(iv) a CDR-H1 comprising the amino acid sequence shown in SEQ ID NO: 26, a CDR-H2 comprising the amino acid sequence shown in SEQ ID NO: 30, and a CDR-H3 comprising the amino acid sequence shown in SEQ ID NO: 33, where the CDRs are defined by the IMGT numbering system.

Embodiment 4. The antibody-drug conjugate of any one of embodiments 2-3, wherein (i) the first light chain variable region (VL) comprises the amino acid sequence shown in SEQ ID NO: 17 and the first heavy chain variable region (VH) comprises the amino acid sequence shown in SEQ ID NO: 18; or (ii) the first light chain variable region (VL) comprises the amino acid sequence shown in SEQ ID NO: 59 and the first heavy chain variable region (VH) comprises the amino acid sequence shown in SEQ ID NO: 60.

Embodiment 5. The antibody-drug conjugate of any one of embodiments 2-4, wherein the second light chain variable region (VL) comprises the amino acid sequence shown in SEQ ID NO: 15, and/or the second heavy chain variable region (VH) comprises the amino acid sequence shown in SEQ ID NO: 16.

Embodiment 6. The antibody-drug conjugate according to any one of embodiments 1 to 5, wherein the bispecific antibody or its antigen binding fragment further comprises an Fc domain dimer comprising first and second Fc domain monomers, and the first and second Fc domain monomers each independently comprise a modification of one or more amino acids that promotes heterodimerization of the first and second Fc domain monomers.

Embodiment 7. The antibody-drug conjugate of embodiment 6, wherein the Fc comprises a first Fc domain monomer containing amino acid modifications capable of forming a knob structure and a second Fc domain monomer containing amino acid modifications capable of forming a hole structure, wherein the hole structure is capable of pairing with the knob structure to form a heterodimeric Fc domain dimer.

Embodiment 8. The antibody-drug conjugate of embodiment 6 or 7, wherein (a) the first Fc domain monomer comprises the amino acid sequence shown in SEQ ID NO: 49, and the second Fc domain monomer comprises the amino acid sequence shown in SEQ ID NO: 50; or (b) the first Fc domain monomer comprises the amino acid sequence shown in SEQ ID NO: 51, and the second Fc domain monomer comprises the amino acid sequence shown in SEQ ID NO: 52.

Embodiment 9. The antibody-drug conjugate according to any one of embodiments 6-8, wherein the first antigen-binding domain and the first Fc domain monomer are connected and the second antigen-binding domain is connected to the second Fc domain monomer; or the first antigen binding domain and the second Fc domain monomer are connected and the second antigen-binding domain is connected to the first Fc domain monomer.

Embodiment 10. The antibody-drug conjugate described in any one of embodiments 1-9, wherein the first antigen-binding domain is a Fab and the second antigen-binding domain is an scFv.

Embodiment 11. The antibody-drug conjugate of embodiment 10, wherein the bispecific antibody comprises polypeptide chain I-A, polypeptide chain I-B, and polypeptide chain I-C; wherein,
(a) the elements comprised in polypeptide chain I-A from the N-terminal end to the C-terminal end comprise the first VL and CL; the elements comprised in polypeptide chain I-B from the N-terminal end to the C-terminal end comprise the first VH, the heavy chain CH1 region, and the first Fc domain monomer; and/or the elements comprised in polypeptide chain I-C from the N-terminal end to the C-terminal end comprise (i) the second VL, the second VH, and the second Fc domain monomer; or (ii) the second VH, the second VL, and the second Fc domain monomer; or
(b) the elements comprised in polypeptide chain I-A from the N-terminal end to the C-terminal end comprise the first VL and CL; the elements comprised in polypeptide chain I-B from the N-terminal end to the C-terminal end comprise the first VH, the heavy chain CH1 region, and the second Fc domain monomer; and/or the elements comprised in polypeptide chain I-C from the N-terminal end to the C-terminal end comprise (i) the second VL, the second VH, and the first Fc domain monomer; or (ii) the second VH, the second VL, and the first Fc domain monomer.

Embodiment 12. The antibody-drug conjugate of embodiment 10 or 11, wherein (a) the polypeptide chain I-A comprises the amino acid sequence shown in SEQ ID NO: 1; the polypeptide chain I-B comprises the amino acid sequence shown in SEQ ID NO: 2; and/or the polypeptide chain I-C comprises the amino acid sequence shown in SEQ ID NO: 3; or (b) the polypeptide chain I-A comprises the amino acid sequence shown in SEQ ID NO: 1; the polypeptide chain I-B comprises the amino acid sequence shown in SEQ ID NO: 9; and/or the polypeptide chain I-C comprises the amino acid sequence shown in SEQ ID NO: 10.

Embodiment 13. The antibody-drug conjugate according to any one of embodiments 1 to 9, wherein the first antigen-binding domain is an scFv, and the second antigen-binding domain is a Fab.

Embodiment 14. The antibody-drug conjugate of embodiment 13, wherein
(a) the bispecific antibody comprises polypeptide chain II-A, polypeptide chain II-B, and polypeptide chain II-C; wherein, the elements comprised in polypeptide chain II-A from the N-terminal end to the C-terminal end comprise the second VL and the CL; the elements comprised in polypeptide chain II-B from the N-terminal end to the C-terminal end comprise the second VH, the heavy chain CH1 region, and the first Fc domain monomer; and/or the elements comprised in polypeptide chain II-C from N-terminal end to C-terminal end comprise:
   (i) the first VL, the first VH, and the second Fc domain monomer; or
   (ii) the first VH, the first VL, and the second Fc domain monomer; or
(b) the bispecific antibody comprises polypeptide chain II-A, polypeptide chain II-B, and polypeptide chain II-C; wherein, the elements comprised in polypeptide chain II-A from the N-terminal end to the C-terminal end comprise the second VL and the CL; the elements comprised in polypeptide chain II-B from the N-terminal end to the C-terminal end comprise the second VH, the heavy chain CH1 region, and the second Fc domain monomer; and/or the elements comprised in polypeptide chain II-C from the N-terminal end to the C-terminal end comprise:
   (i) the first VL, the first VH, and the first Fc domain monomer; or
   (ii) the first VH, the first VL, and the first Fc domain monomer.

Embodiment 15. The antibody-drug conjugate of embodiment 13 or 14, wherein (a) the polypeptide chain II-A comprises the amino acid sequence shown in SEQ ID NO: 4; the polypeptide chain II-B comprises the amino acid sequence shown in SEQ ID NO: 5; and/or the polypeptide chain II-C comprises the amino acid sequence shown in SEQ ID NO: 6; or (b) the polypeptide chain II-A comprises the amino acid sequence shown in SEQ ID NO: 4; the polypeptide chain II-B comprises the amino acid sequence shown in SEQ ID NO: 7; and/or the polypeptide chain II-C comprises the amino acid sequence shown in SEQ ID NO: 8.

Embodiment 16. The antibody-drug conjugate of any one of embodiments 1-9, wherein the first antigen-binding domain is an scFab, and the second antigen-binding domain is a Fab.

Embodiment 17. The antibody-drug conjugate of embodiment 16, wherein
(a) the bispecific antibody comprises polypeptide chain IV-A, polypeptide chain IV-B, and polypeptide chain IV-C; wherein the elements comprised in polypeptide chain IV-A from the N-terminal end to the C-terminal end comprise the second VL and the CL; the elements comprised in polypeptide chain IV-B from the N-terminal end to the C-terminal end comprise the second VH, the heavy chain CH1 region, and the second Fc domain monomer; and/or the elements comprised in polypeptide chain IV-C from the N-terminal end to the C-terminal end comprise:
   (i) the first VL, the CL, the first VH, the heavy chain CH1 region, and the first Fc domain monomer; or
   (ii) the first VH, the heavy chain CH1 region, the first VL, the CL, and the first Fc domain monomer; or
(b) the bispecific antibody comprises polypeptide chain IV-A, polypeptide chain IV-B, and polypeptide chain IV-C; wherein the elements comprised in polypeptide chain IV-A from the N-terminal end to the C-terminal end comprise the second VL and the CL; the elements comprised in polypeptide chain IV-B from the N-terminal end to the C-terminal end comprise the second VH, the heavy chain CH1 region, and the second Fc domain monomer; and/or the elements comprised in polypeptide chain IV-C from the N-terminal end to the C-terminal end comprise:
   (i) the first VL, the CL, the first VH, the heavy chain CH1 region, and the first Fc monomer; or
   (ii) the first VH, the heavy chain CH1 region, the first VL, the CL, and the first Fc domain monomer.

Embodiment 18. The antibody-drug conjugate of embodiment 16 or 17, wherein the polypeptide chain IV-A comprises the amino acid sequence shown in SEQ ID NO: 4; the polypeptide chain IV-B comprises the amino acid sequence shown in SEQ ID NO: 7; and/or the polypeptide chain IV-C comprises the amino acid sequence shown in SEQ ID NO: 11.

Embodiment 19. The antibody-drug conjugate of any one of embodiments 1-9, wherein the first antigen-binding domain and the second antigen-binding domain are each a Fab, and wherein the second antigen-binding domain Fab comprises a CrossMab form of domain swapping.

Embodiment 20. The antibody-drug conjugate of embodiment 19, wherein
(a) the bispecific antibody comprises polypeptide chain V-A, polypeptide chain V-B, polypeptide chain V-C, and polypeptide chain V-D; wherein the elements comprised in polypeptide chain V-A from the N-terminal end to the C-terminal end comprise the first VL and CL; the elements comprised in polypeptide chain V-B from the N-terminal end to the C-terminal end comprise the first VH, the heavy chain CH1 region, and the first Fc domain monomer; the elements comprised in polypeptide chain V-C from the N-terminal end to the C-terminal end comprise the second VH, the CL, and the second Fc domain monomer; and/or the elements comprised in polypeptide chain V-D from the N-terminal end to the C-terminal end comprise the second VL and heavy chain CH1 region; or
(b) the bispecific antibody comprises polypeptide chain V-A, polypeptide chain V-B, polypeptide chain V-C, and polypeptide chain V-D; wherein the elements comprised in polypeptide chain V-A from the N-terminal end to the C-terminal end comprise the first VL and the CL; the elements comprised in polypeptide chain V-B from the N-terminal end to the C-terminal end comprise the first VH, the heavy chain CH1 region, and the second Fc domain monomer; the elements comprised in polypeptide chain V-C from the N-terminal end to the C-terminal end comprise the second VH, the CL, and the first Fc domain monomer; and/or the elements comprised in polypeptide chain V-D from the N-terminal end to the C-terminal end comprise the second VL and heavy chain CH1 region.

Embodiment 21. The antibody-drug conjugate of embodiment 19 or 20, wherein the polypeptide chain V-A comprises the amino acid sequence shown in SEQ ID NO: 1; the polypeptide chain V-B comprises the amino acid sequence shown in SEQ ID NO: 9; the polypeptide chain V-C comprises the amino acid sequence shown in SEQ ID NO: 13; and/or the polypeptide chain V-D comprises the amino acid sequence shown in SEQ ID NO: 12.

Embodiment 22. The antibody-drug conjugate of any one of embodiments 1-9, wherein both the first antigen-binding domain and the second antigen-binding domain are scFv.

Embodiment 23. The antibody-drug conjugate of embodiment 22, wherein
(a) the bispecific antibody comprises polypeptide chain VII-A and polypeptide chain VII-B; wherein the elements comprised in polypeptide chain VII-A from the N-terminal end to the C-terminal end comprise:
   (i) the first VL, the first VH, and the first Fc domain monomer, or
   (ii) the first VH, the first VL, and the first Fc domain monomer; and/or the elements comprised in polypeptide chain VII-B rom the N-terminal end to the C-terminal end comprise:
   (x) the second VL, the second VH, and said second Fc domain monomer, or
   (y) the second VH, the second VL, and the second Fc domain monomer; or
(b) the bispecific antibody comprises polypeptide chain VII-A and polypeptide chain VII-B; wherein the polypeptide chain VII-A from the N-terminal end to the C-terminal end comprise:
   (i) the first VL, the first VH, and the second Fc domain monomer, or
   (ii) the first VH, the first VL, and the second Fc domain monomer; and/or the elements comprised in polypeptide chain VII-B contains from the N-terminal end to the C-terminal end comprise:
   (x) the second VL, the second VH, and said first Fc domain monomer, or
   (y) the second VH, the second VL, and the first Fc domain monomer.

Embodiment 24. The antibody-drug conjugate of embodiment 22 or 23, wherein the polypeptide chain VII-A comprises the amino acid sequence shown in SEQ ID NO: 14, and/or the polypeptide chain VII-B comprises the amino acid sequence shown in SEQ ID NO: 8.

Embodiment 25. The antibody-drug conjugate of embodiment 11, 14, 17, 20 or 23, wherein adjacent elements comprising each of the polypeptide chains are optionally connected through a peptide linker or not.

Embodiment 26. The antibody-drug conjugate of embodiment 25, wherein each peptide linker is independently the same peptide linker or a different peptide linker, wherein each peptide linker selected from the group consisting of a rigid peptide linker and a flexible peptide linker.

Embodiment 27. The antibody-drug conjugate of embodiment 26, wherein each peptide linker is independently selected from peptide linkers comprising one or more glycine (G) and/or serine (S) residues.

Embodiment 28. The antibody-drug conjugate of embodiment 27, wherein each peptide linker independently comprises 1, 2, 3, 4, 5, 6, 7, 8, 9 or 10 tandem copies of a peptide linker subunit comprising the amino acid sequence GGGGS (SEQ ID NO: 57).

Embodiment 29. The antibody-drug conjugate of embodiment 28, wherein each peptide linker independently comprises the amino acid sequence shown in SEQ ID NO: 55 or 56.

Embodiment 30. The antibody-drug conjugate of embodiment 11, 14, 17, 20 or 23, wherein the CL comprises the amino acid sequence shown in SEQ ID NO: 53 or SEQ ID NO: 63; and/or the heavy chain CH1 region comprises the amino acid sequence shown in SEQ ID NO: 54.

Embodiment 31. The antibody-drug conjugate according to any one of embodiments 1 to 30, wherein the bispecific antibody or its antigen-binding fragment comprises:
(a) polypeptide chain I-A comprising the amino acid sequence shown in SEQ ID NO: 1, polypeptide chain I-B comprising the amino acid sequence shown in SEQ ID NO: 2 or 9, and/or polypeptide chain I-C comprising the amino acid sequence shown in SEQ ID NO: 3 or 10;
(b) polypeptide chain II-A comprising the amino acid sequence shown in SEQ ID NO: 4, polypeptide chain II-B comprising the amino acid sequence shown in SEQ ID NO: 5 or 7, and/or polypeptide chain II-C comprising the amino acid sequence shown in SEQ ID NO: 6 or 8;
(c) polypeptide chain IV-A comprising the amino acid sequence shown in SEQ ID NO: 4, polypeptide chain IV-B comprising the amino acid sequence shown in SEQ ID NO: 7, and/or polypeptide chain IV-C comprising the amino acid sequence shown in SEQ ID NO: 11;
(d) polypeptide chain V-A comprising the amino acid sequence shown in SEQ ID NO: 1, polypeptide chain V-B comprising the amino acid sequence shown in SEQ ID NO: 9, polypeptide chain V-C comprising the amino acid sequence shown in SEQ ID NO: 13, and/or polypeptide chain V-D comprising the amino acid sequence shown in SEQ ID NO: 12; or
(e) polypeptide chain VII-A comprises the amino acid sequence shown in SEQ ID NO: 14, and/or polypeptide chain VII-B comprising the amino acid sequence of SEQ ID NO: 8.

Embodiment 32. The antibody-drug conjugate of embodiment 28, wherein the bispecific antibody or antigen-binding fragment thereof comprises:
(a) the polypeptide chain I-A comprising the amino acid sequence shown in SEQ ID NO: 1, the polypeptide chain I-B comprising the amino acid sequence shown in SEQ ID NO: 2, and the polypeptide chain I-C comprising the amino acid sequence shown in SEQ ID NO: 3;
(b) the polypeptide chain I-A comprising the amino acid sequence shown in SEQ ID NO: 1, the polypeptide chain I-B comprising the amino acid sequence shown in SEQ ID NO: 9, and the polypeptide chain I-C comprising the amino acid sequence shown in SEQ ID NO: 10;
(c) the polypeptide chain II-A comprising the amino acid sequence shown in SEQ ID NO: 4, the polypeptide chain II-B comprising the amino acid sequence shown in SEQ ID NO: 5, and the polypeptide chain II-C comprising the amino acid sequence shown in SEQ ID NO: 6;
(d) the polypeptide chain II-A comprising the amino acid sequence shown in SEQ ID NO: 4, the polypeptide chain II-B comprising the amino acid sequence shown in 7SEQ ID NO: and the polypeptide chain II-C comprising the amino acid sequence shown in SEQ ID NO: 8;
(e) the polypeptide chain IV-A comprising the amino acid sequence shown in SEQ ID NO: 4, the polypeptide chain IV-B comprising the amino acid sequence shown in SEQ ID NO: 7, and the polypeptide chain IV-C with the amino acid sequence shown in SEQ ID NO: 11;
(f) the polypeptide chain V-A comprising the amino acid sequence shown in SEQ ID NO: 1, the polypeptide chain V-B comprising the amino acid sequence shown in SEQ ID NO: 9, the polypeptide chain V-C comprising the amino acid sequence shown in SEQ ID NO: 13, and the polypeptide chain V-D comprising the amino acid sequence shown in SEQ ID NO: 12; or
(g) the polypeptide chain VII-A comprising the amino acid sequence shown in SEQ ID NO: 14, and the polypeptide chain VII-B comprising the amino acid sequence shown in SEQ ID NO: 8.

Embodiment 33. The antibody-drug conjugate according to any one of embodiments 1 to 30, wherein the bispecific antibody or its antigen-binding fragment has an enhanced tumor inhibitory effect compared to the monospecific anti-c-MET antibody and/or the monospecific anti-EGFR antibody; and wherein
(a) the amino acid sequences of the CDRs of the monospecific anti-c-MET antibody are the same as the amino acid sequences of the CDRs of the first antigen-binding domain and the amino acid sequence of the CDRs of the monospecific anti-EGFR antibody are the same as the amino acid sequences of the CDRs of the second antigen-binding domain, or
(b) the amino acid sequences of the CDRs of the monospecific anti-c-MET antibody are the same as the amino acid sequences of the CDRs of the second antigen-binding domain and the amino acid sequence of the CDRs of the monospecific anti-EGFR antibody are the same as the amino acid sequences of the CDRs of the first antigen-binding domain.

Embodiment 34. The antibody-drug conjugate of embodiment 33, wherein the tumor inhibitory effect includes inhibition of the EGFR and c-MET signaling pathways, antibody-dependent cell-mediated cytotoxicity (ADCC) activity, and/or complement-dependent cytotoxicity (CDC) activity.

Embodiment 35. The antibody-drug conjugate according to any one of embodiments 1-34, wherein M comprises

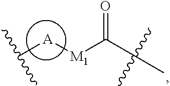

and wherein ring A is a 5-6 membered aliphatic heterocyclic ring, or a 5-20 membered aromatic ring system, and the aliphatic heterocyclic ring and aromatic ring system are optionally replaced by one or more groups independently selected from the group consisting of oxo (═O), halogen, cyano, amino, carboxyl, thiol, and C₁₋₆ alkyl; M₁ is selected from single bond and C₁₋₂₀ alkylene, C₂₋₂₀ alkenylene, C₂₋₂₀ alkynylene, or amine group.

Embodiment 36. The antibody-drug conjugate according to any one of embodiments 1-34, wherein M comprises

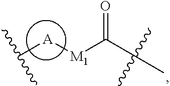

wherein ring A is a 5-membered aliphatic heterocyclic ring, a 6-membered heteroaromatic ring, or a polycyclic ring formed by connecting more than one 6-membered aromatic heterocyclic ring and a benzene ring through a single bond or by connecting more than one 6-membered heteroaromatic rings through single bonds, and wherein the aliphatic heterocyclic ring is optionally substituted by one or more groups selected from oxo (═O), halogen, and C₁₋₄ Alkyl group substitution; M₁ is selected from single bond, C₁₋₂₀ Alkylene, C₂₋₂₀ alkenylene, C₂₋₂₀ alkynylene or amine group.

Embodiment 37. The antibody-drug conjugate according to any one of embodiments 1-34, wherein M is

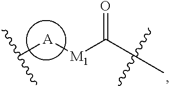

wherein ring A is selected from

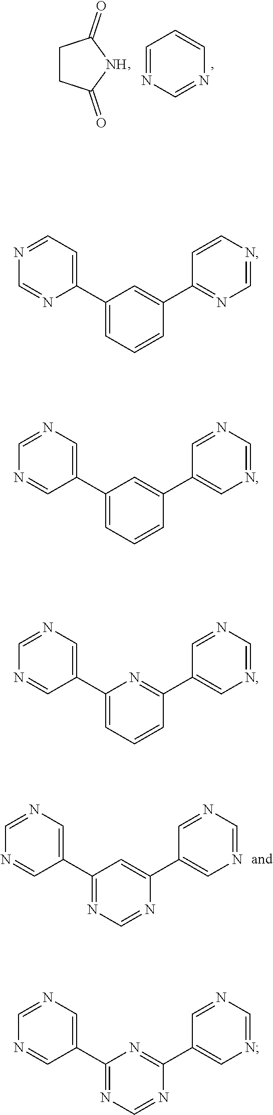

M₁ is selected from a single bond, C₁₋₆ alkylene, C₂₋₆ alkenylene, C₂₋₆ alkynylene, and amine group.

Embodiment 38. The antibody-drug conjugate according to any one of embodiments 1-34, wherein M is selected from

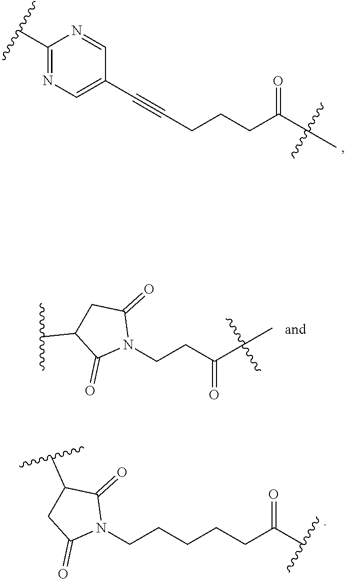 [SMILES: *C(=O)CCCC#Cc1cnc(*)nc1 ; *C(=O)CCN1C(=O)CC(*)C1=O ; *C(=O)CCCCCN1C(=O)CC(*)C1=O]

Embodiment 39. The antibody-drug conjugate according to any one of embodiments 1-34, wherein M is

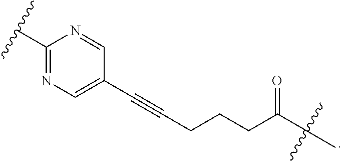 [SMILES: *C(=O)CCCC#Cc1cnc(*)nc1]

Embodiment 40. The antibody-drug conjugate according to any one of embodiments 1-34, wherein M is selected from

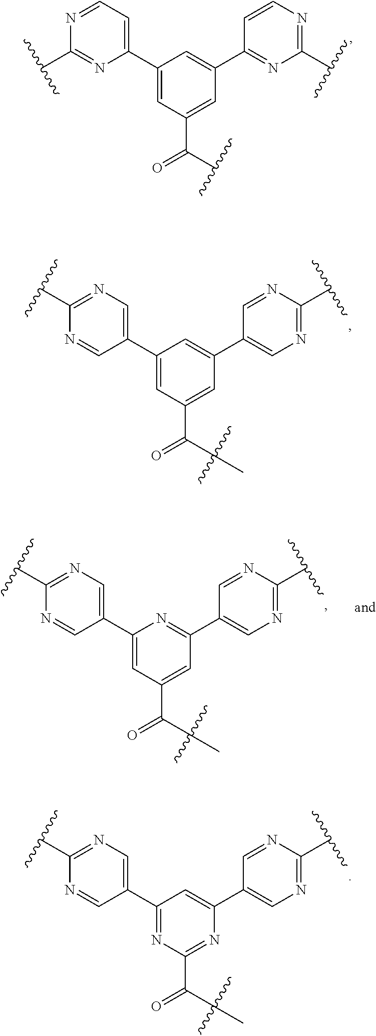 [SMILES: *C(=O)c1cc(-c2ccnc(*)n2)cc(-c2ccnc(*)n2)c1 ; *C(=O)c1cc(-c2cnc(*)nc2)cc(-c2cnc(*)nc2)c1 ; *C(=O)c1cc(-c2cnc(*)nc2)nc(-c2cnc(*)nc2)c1 ; *C(=O)c1nc(-c2cnc(*)nc2)cc(-c2cnc(*)nc2)n1]

Embodiment 41. The antibody-drug conjugate according to any one of embodiments 1-34, wherein M is selected from

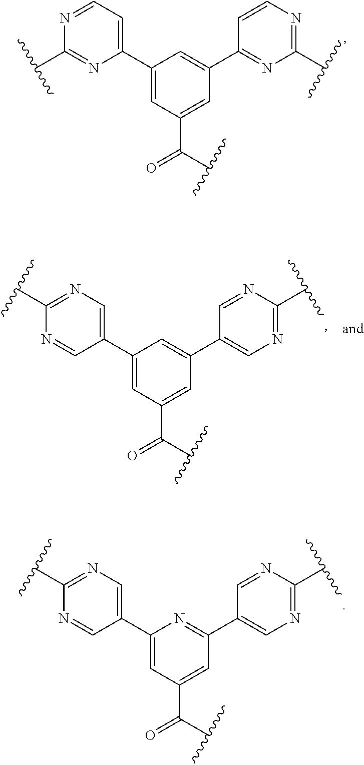 [SMILES: *C(=O)c1cc(-c2ccnc(*)n2)cc(-c2ccnc(*)n2)c1 ; *C(=O)c1cc(-c2cnc(*)nc2)cc(-c2cnc(*)nc2)c1 ; *C(=O)c1cc(-c2cnc(*)nc2)nc(-c2cnc(*)nc2)c1]

Embodiment 42. The antibody-drug conjugate according to any one of embodiments 1-41, wherein L is selected from a structure comprising one or more of the following: C₁₋₆ Alkylene, —N(R′)—, carbonyl, —O—, natural amino acids or unnatural amino acids and their analogs selected from Ala, Arg, Asn, Asp, Cit, Cys, Gln, Glu, Gly, His, Ile, Leu, Lys, Met, Phe, Pro, Ser, Thr, Trp, Tyr, Val, Lys, (COCH₂CH₂(OCH₂CH₂)ₛOCH₃)), and peptides comprising 1, 2, 3, or 4 amino acids,

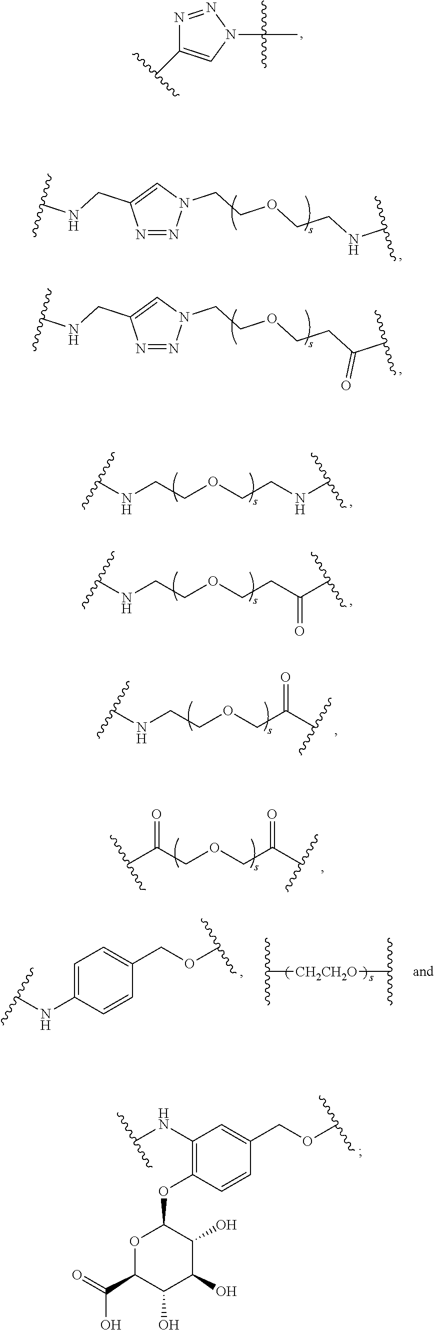

wherein R′ represents hydrogen, C₁₋₆ alkyl, or a polyethylene glycol fragment containing 1-10 ethylene oxide units; and s is selected from an integer of 1-20.

Embodiment 43. The antibody-drug conjugate of embodiment 42, wherein the peptide is selected from Ala-Ala, Ala-Lys, Ala-Lys(Ac), Ala-Pro, Gly-Glu, Gly-Gly, Phe-Lys, Phe-Lys(Ac), Val-Ala, Val-Lys, Val-Lys(Ac), Val-Cit, Ala-Ala-Ala, Ala-Ala-Asn, Leu-Ala-Glu, Gly-Gly-Arg, Gly-Glu-Gly, Gly-Gly-Gly, Gly-Ser-Lys, Glu-Val-Ala, Glu-Val-Cit, Ser-Ala-Pro, Val-Leu-Lys, Val-Lys-Ala, Val-Lys-Gly, Gly-Gly-Phe-Gly (GGFG (SEQ ID NO: 71)), Gly-Gly-Val-Ala (GGVA (SEQ ID NO: 72), Gly-Phe-Leu-Gly (GFLG (SEQ ID NO: 73)), Glu-Ala-Ala-Ala (EAAA (SEQ ID NO: 74)), and Gly-Gly-Gly-Gly-Gly (GGGGG (SEQ ID NO: 75)).

Embodiment 44. The antibody-drug conjugate according to any one of embodiments 1-41, wherein L is selected from a structure comprising one or more of the following: C₁₋₆ Alkylene, carbonyl, —NH—, Ala-Ala, Ala-Lys, Ala-Pro, Gly-Glu, Gly-Gly, Phe-Lys, Val-Ala, Val-Lys, Val-Cit, Ala-Ala-Ala, Ala-Ala-Asn, Leu-Ala-Glu, Gly-Gly-Arg, Gly-Glu-Gly, Gly-Gly-Gly, Gly-Ser-Lys, Glu-Val-Ala, Glu-Val-Cit, Ser-Ala-Pro, Val-Leu-Lys, Val-Lys-Ala, Val-Lys-Gly, Gly-Gly-Phe-Gly (GGFG (SEQ ID NO: 71)), Gly-Gly-Val-Ala (GGVA (SEQ ID NO: 72), Gly-Phe-Leu-Gly (GFLG (SEQ ID NO: 73)), Glu-Ala-Ala-Ala (EAAA (SEQ ID NO: 74)), and Gly-Gly-Gly-Gly-Gly (GGGGG (SEQ ID NO: 75)),

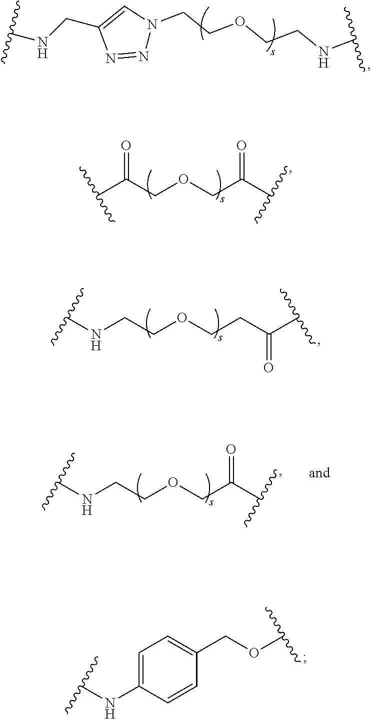

wherein s is selected from an integer of 1-20.

Embodiment 45. The antibody-drug conjugate according to any one of embodiments 1-41, wherein L is selected from a structure consisting of one or more of the following:

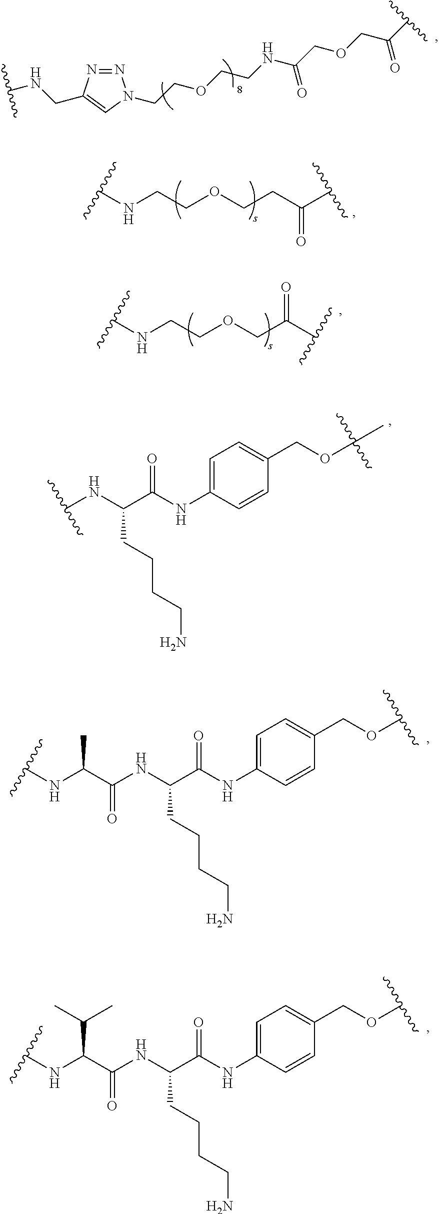

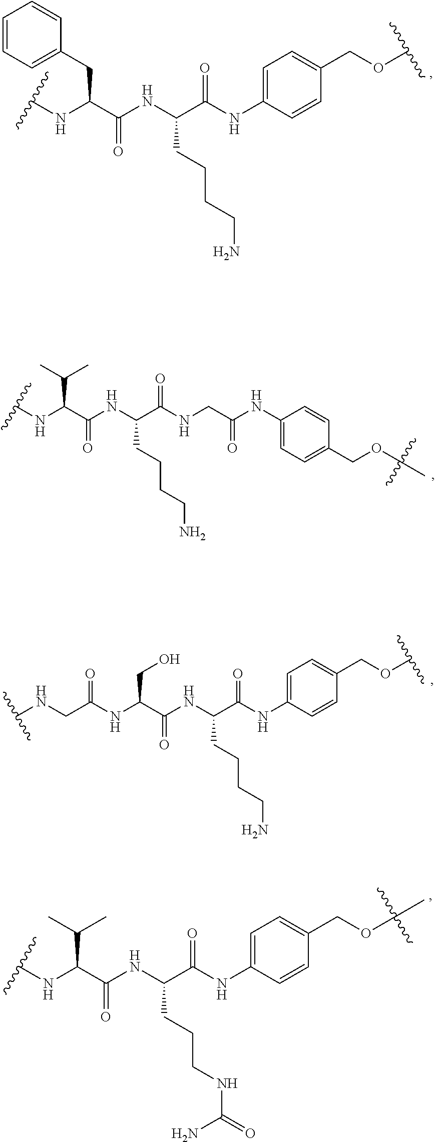 [SMILES: *N[C@@H](Cc1ccccc1)C(=O)N[C@@H](CCCCN)C(=O)Nc1ccc(CO*)cc1 ; *N[C@H](C(=O)N[C@@H](CCCCN)C(=O)NCC(=O)Nc1ccc(CO*)cc1)C(C)C ; *NCC(=O)N[C@@H](CO)C(=O)N[C@@H](CCCCN)C(=O)Nc1ccc(CO*)cc1 ; *N[C@H](C(=O)N[C@@H](CCCNC(N)=O)C(=O)Nc1ccc(CO*)cc1)C(C)C]

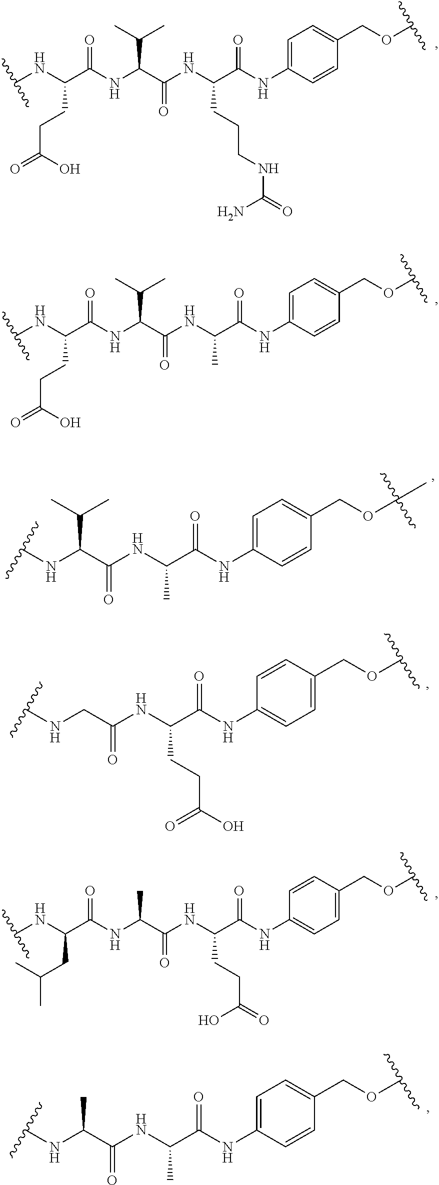 [SMILES: *N[C@@H](CCC(=O)O)C(=O)N[C@H](C(=O)N[C@@H](CCCNC(N)=O)C(=O)Nc1ccc(CO*)cc1)C(C)C ; *N[C@@H](CCC(=O)O)C(=O)N[C@H](C(=O)N[C@@H](C)C(=O)Nc1ccc(CO*)cc1)C(C)C ; *N[C@H](C(=O)N[C@@H](C)C(=O)Nc1ccc(CO*)cc1)C(C)C ; *NCC(=O)N[C@@H](CCC(=O)O)C(=O)Nc1ccc(CO*)cc1 ; *N[C@H](CC(C)C)C(=O)N[C@@H](C)C(=O)N[C@@H](CCC(=O)O)C(=O)Nc1ccc(CO*)cc1 ; *N[C@@H](C)C(=O)N[C@@H](C)C(=O)Nc1ccc(CO*)cc1]

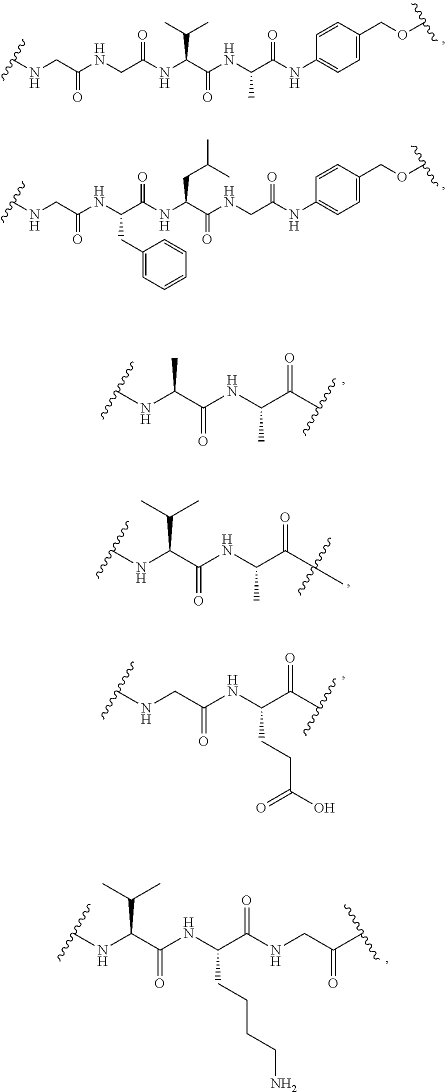 [SMILES: *NCC(=O)NCC(=O)N[C@H](C(=O)N[C@@H](C)C(=O)Nc1ccc(CO*)cc1)C(C)C ; *NCC(=O)N[C@@H](Cc1ccccc1)C(=O)N[C@@H](CC(C)C)C(=O)NCC(=O)Nc1ccc(CO*)cc1 ; *N[C@@H](C)C(=O)N[C@@H](C)C(*)=O ; *N[C@H](C(=O)N[C@@H](C)C(*)=O)C(C)C ; *NCC(=O)N[C@@H](CCC(=O)O)C(*)=O ; *N[C@H](C(=O)N[C@@H](CCCCN)C(=O)NCC(*)=O)C(C)C]

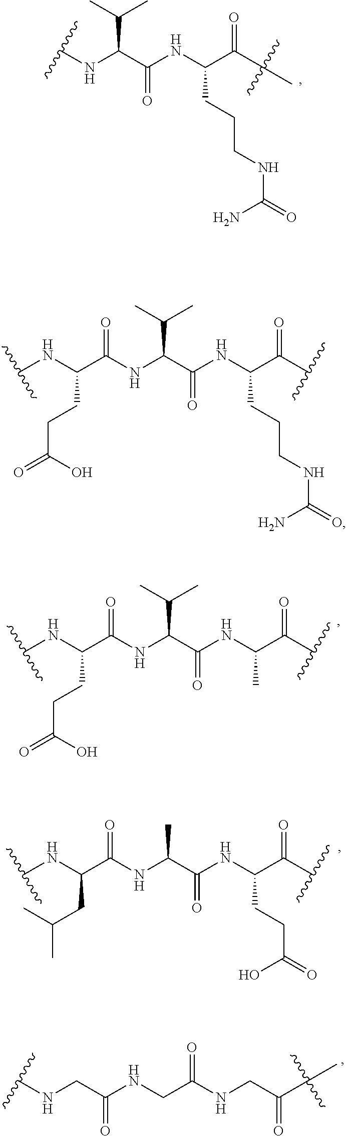 [SMILES: *N[C@H](C(=O)N[C@@H](CCCNC(N)=O)C(*)=O)C(C)C ; *N[C@@H](CCC(=O)O)C(=O)N[C@H](C(=O)N[C@@H](CCCNC(N)=O)C(*)=O)C(C)C ; *N[C@@H](CCC(=O)O)C(=O)N[C@H](C(=O)N[C@@H](C)C(*)=O)C(C)C ; *N[C@H](CC(C)C)C(=O)N[C@@H](C)C(=O)N[C@@H](CCC(=O)O)C(*)=O ; *NCC(=O)NCC(=O)NCC(*)=O]

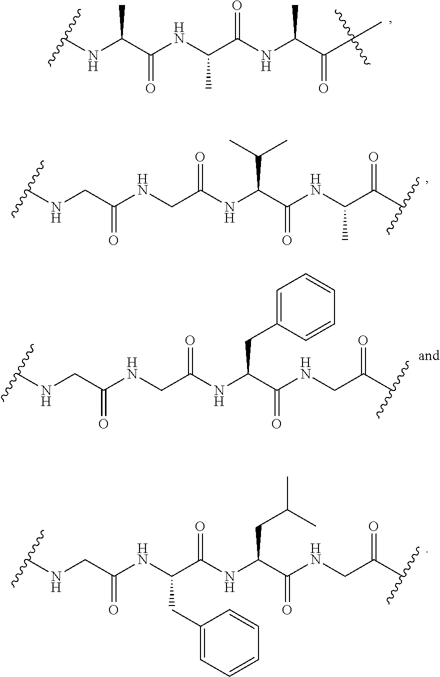

Embodiment 46. The antibody-drug conjugate according to any one of embodiments 1-41, wherein L is selected from the following structures:

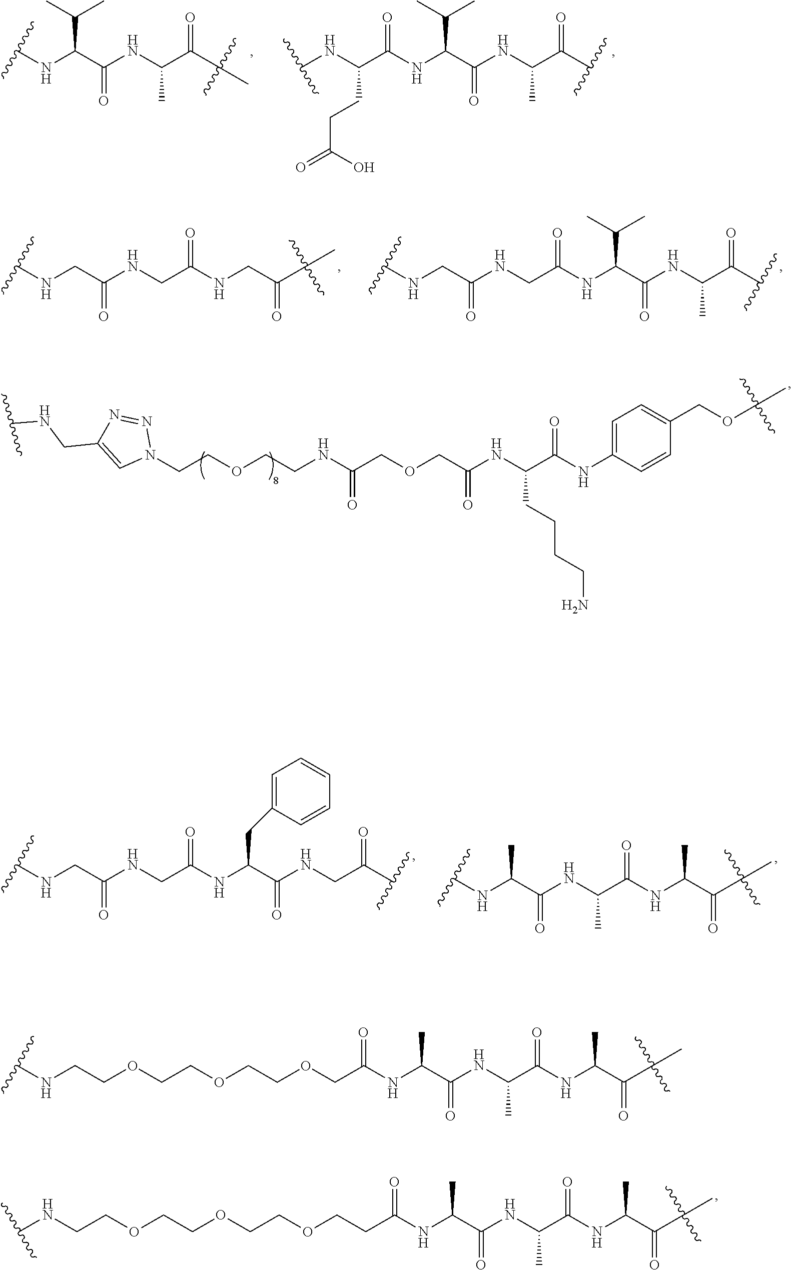

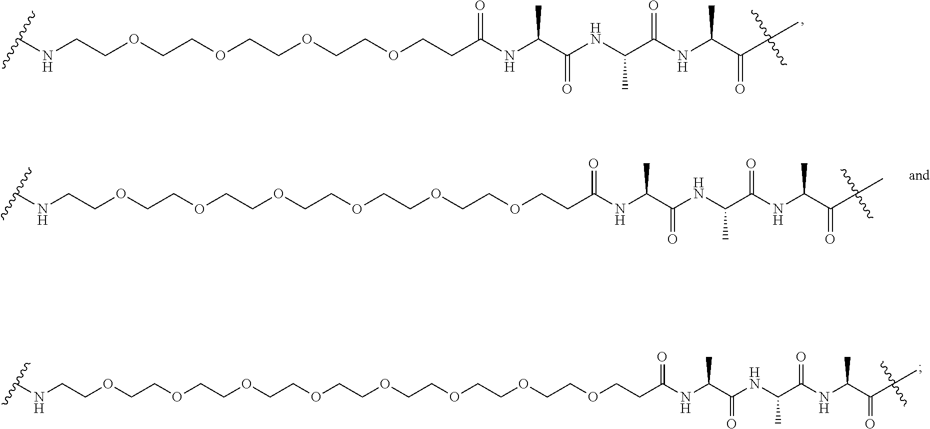 [SMILES: *NCCOCCOCCOCCOCCC(=O)N[C@@H](C)C(=O)N[C@@H](C)C(=O)N[C@@H](C)C(*)=O ; *NCCOCCOCCOCCOCCOCCOCCC(=O)N[C@@H](C)C(=O)N[C@@H](C)C(=O)N[C@@H](C)C(*)=O ; *NCCOCCOCCOCCOCCOCCOCCOCCOCCC(=O)N[C@@H](C)C(=O)N[C@@H](C)C(=O)N[C@@H](C)C(*)=O]

Embodiment 47. The antibody-drug conjugate according to any one of embodiments 1-41, wherein L is selected from the following structures:

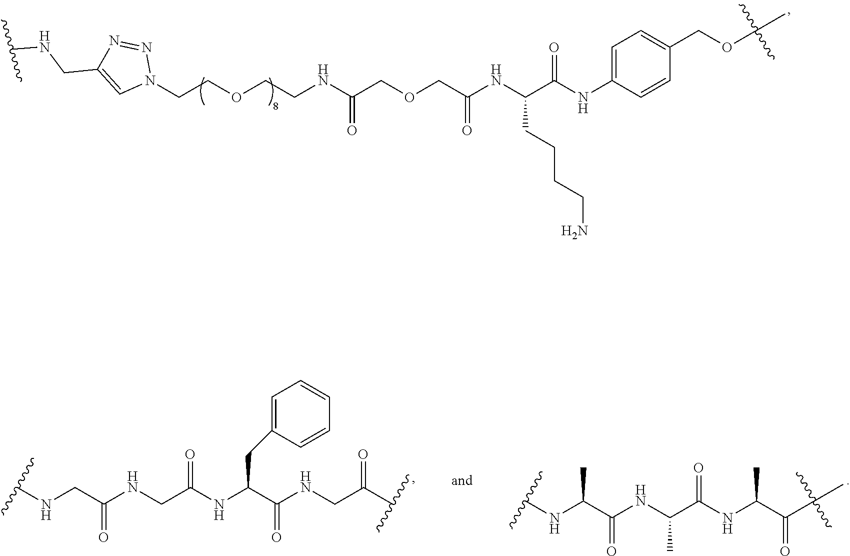

Embodiment 48. The antibody-drug conjugate according to any one of embodiments 1-41, wherein L is selected from the following structures

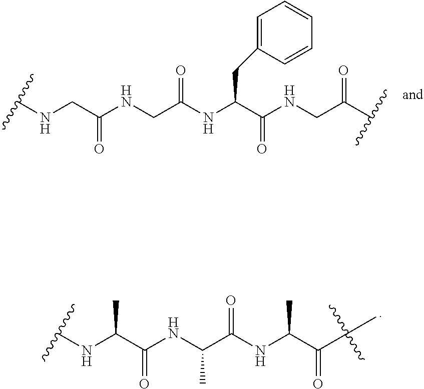 [SMILES: *NCC(=O)NCC(=O)N[C@@H](Cc1ccccc1)C(=O)NCC(*)=O ; *N[C@@H](C)C(=O)N[C@@H](C)C(=O)N[C@@H](C)C(*)=O]

Embodiment 49. The antibody-drug conjugate according to any one of embodiments 1-41, wherein L is selected from the following structures:

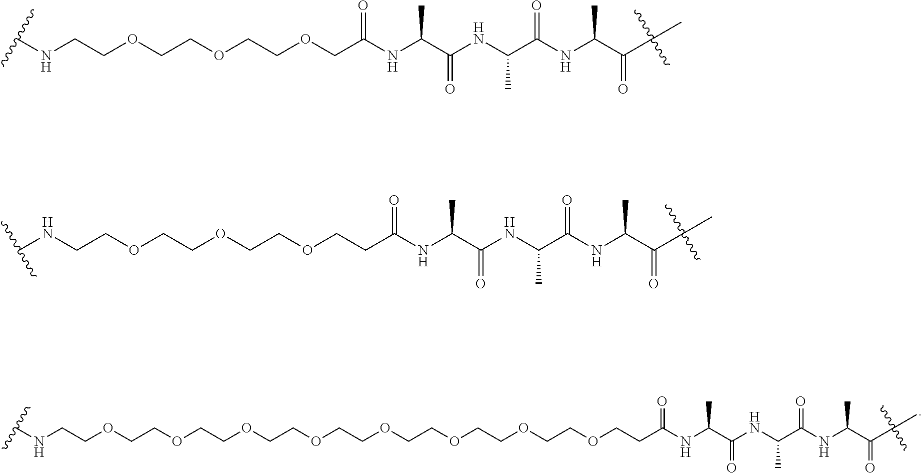 [SMILES: *NCCOCCOCCOCC(=O)N[C@@H](C)C(=O)N[C@@H](C)C(=O)N[C@@H](C)C(*)=O ; *NCCOCCOCCOCCC(=O)N[C@@H](C)C(=O)N[C@@H](C)C(=O)N[C@@H](C)C(*)=O ; *NCCOCCOCCOCCOCCOCCOCCOCCOCCC(=O)N[C@@H](C)C(=O)N[C@@H](C)C(=O)N[C@@H](C)C(*)=O]

Embodiment 50. The antibody-drug conjugate of any one of embodiments 1-49, wherein E is a single bond or selected from the following structures: —NHCH₂—,

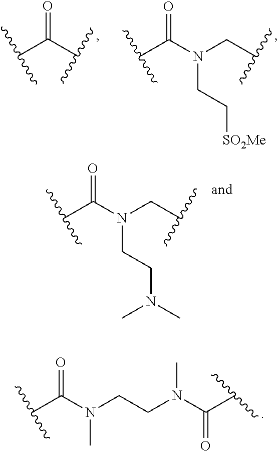

Embodiment 51. The antibody-drug conjugate of any one of embodiments 1-49, wherein E is a single bond, —NHCH₂—,

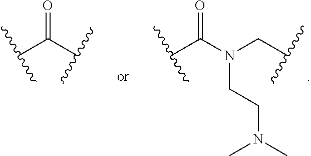

Embodiment 52. The antibody-drug conjugate of any one of embodiments 1-49, wherein E is —NHCH₂— or

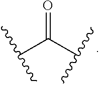 [SMILES: *C(*)=O]

Embodiment 53. The antibody-drug conjugate of any one of embodiments 1-49, wherein E is —NHCH₂—; Preferably, E is a single bond.

Embodiment 54. The antibody-drug conjugate of any one of embodiments 1-49, wherein E is

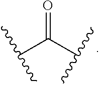 [SMILES: *C(*)=O]

Embodiment 55. The antibody-drug conjugate of described in any one of embodiments 1-54, wherein

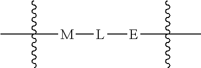

is selected from the following structures:

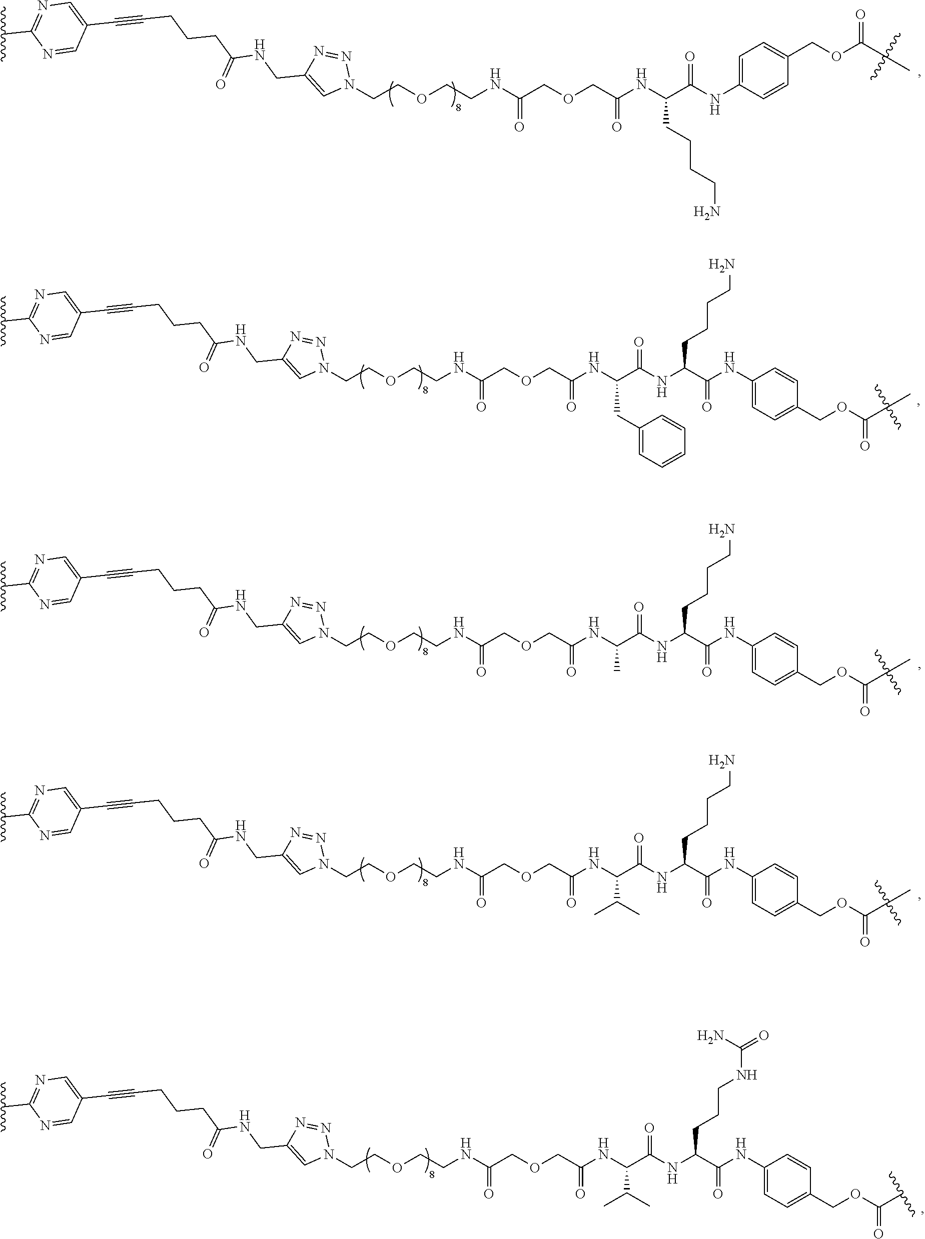

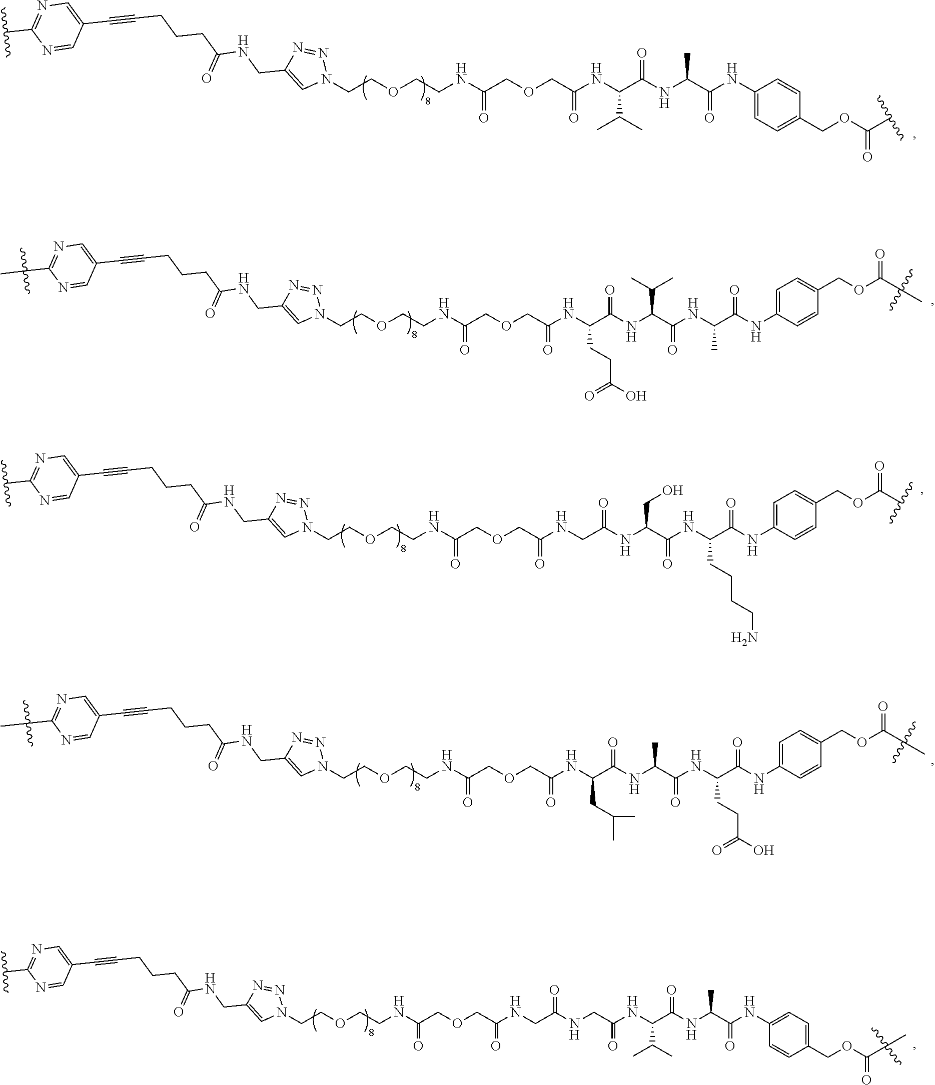

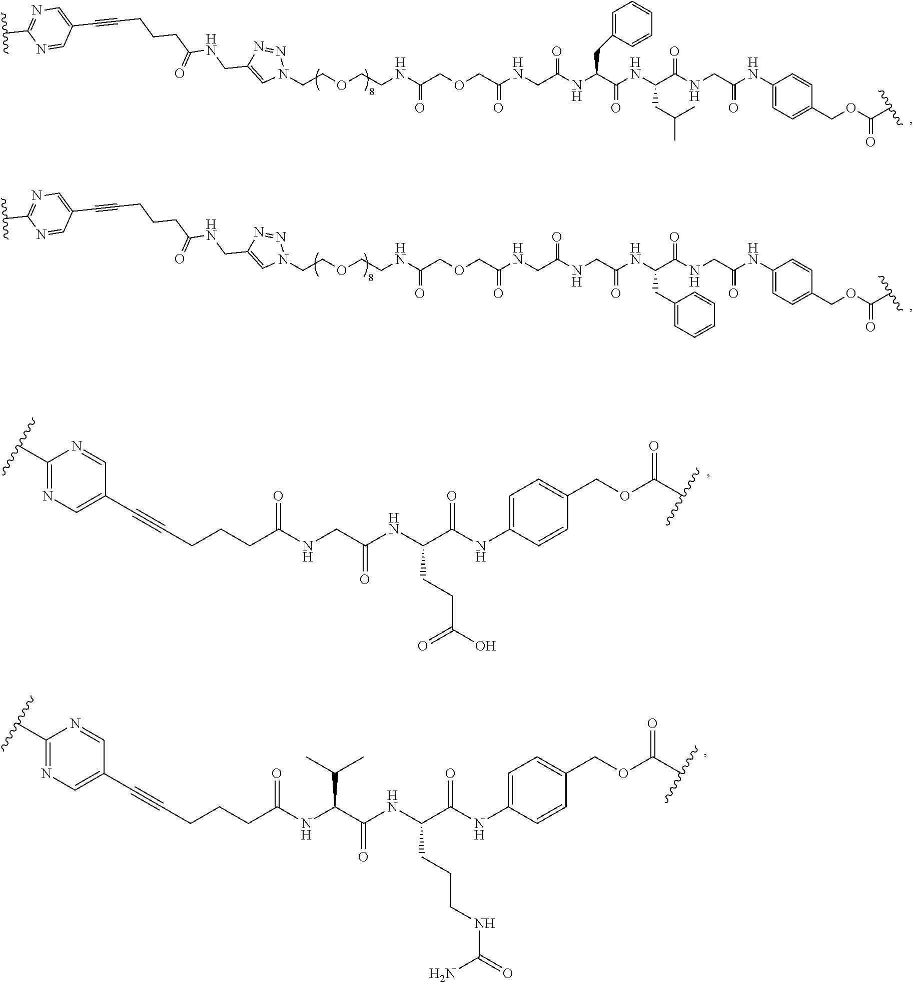

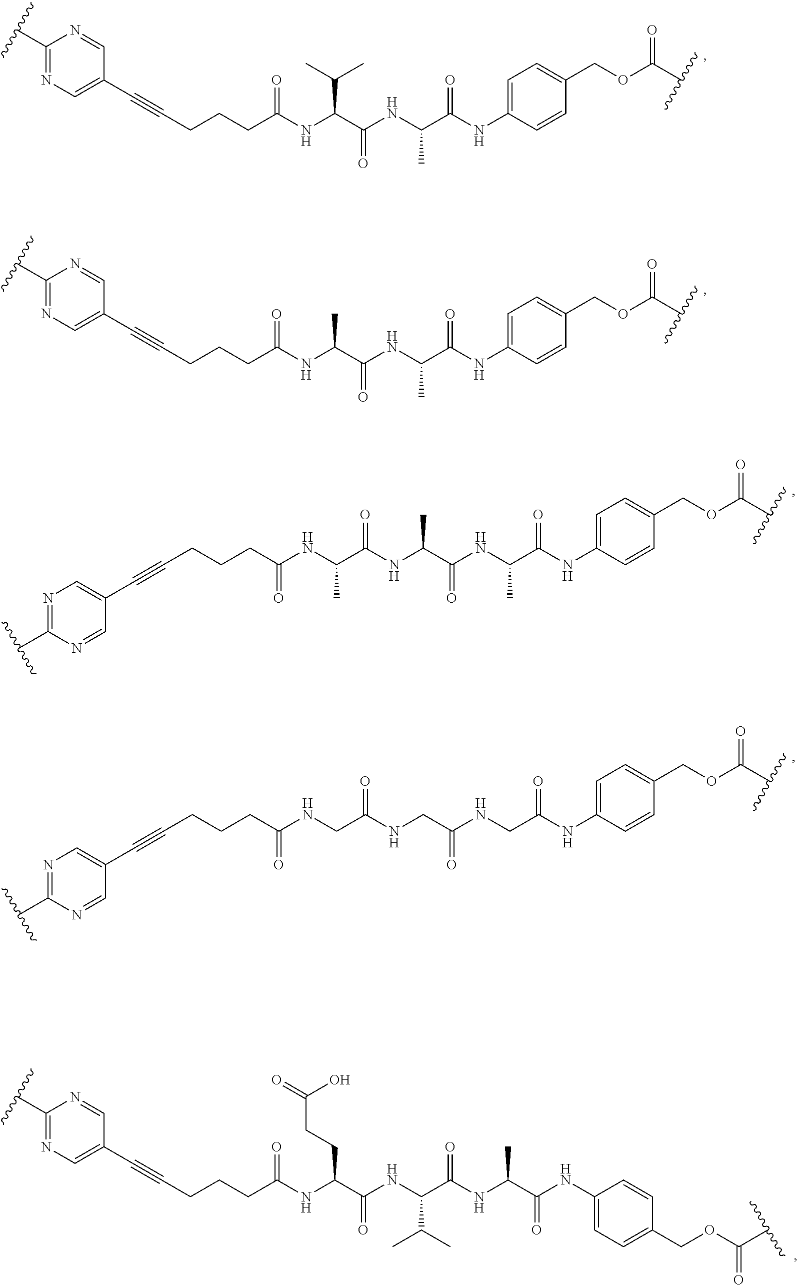 [SMILES: *C(=O)OCc1ccc(NC(=O)[C@H](C)NC(=O)[C@@H](NC(=O)CCCC#Cc2cnc(*)nc2)C(C)C)cc1 ; *C(=O)OCc1ccc(NC(=O)[C@H](C)NC(=O)[C@H](C)NC(=O)CCCC#Cc2cnc(*)nc2)cc1 ; *C(=O)OCc1ccc(NC(=O)[C@H](C)NC(=O)[C@H](C)NC(=O)[C@H](C)NC(=O)CCCC#Cc2cnc(*)nc2)cc1 ; *C(=O)OCc1ccc(NC(=O)CNC(=O)CNC(=O)CNC(=O)CCCC#Cc2cnc(*)nc2)cc1 ; *C(=O)OCc1ccc(NC(=O)[C@H](C)NC(=O)[C@@H](NC(=O)[C@H](CCC(=O)O)NC(=O)CCCC#Cc2cnc(*)nc2)C(C)C)cc1]

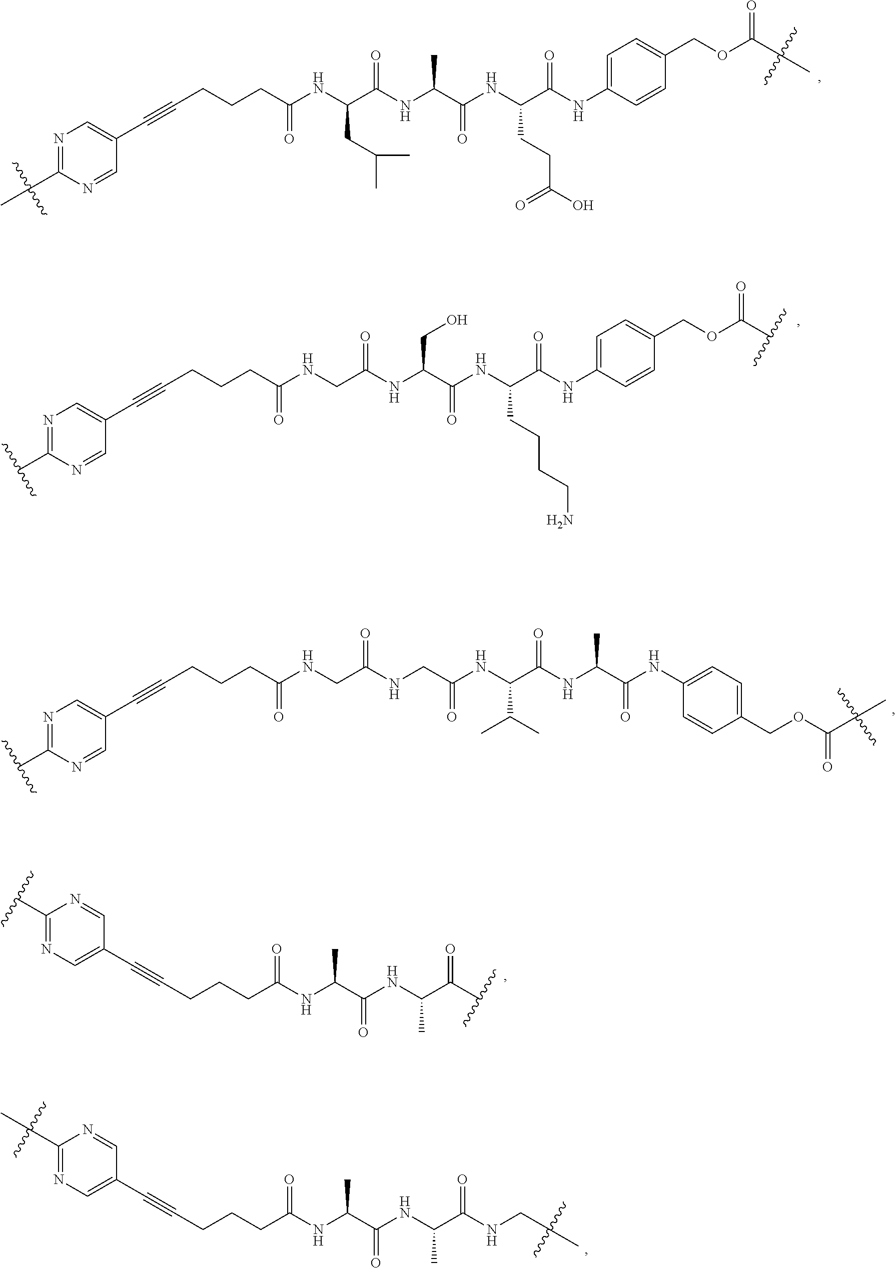 [SMILES: *C(=O)OCc1ccc(NC(=O)[C@H](CCC(=O)O)NC(=O)[C@H](C)NC(=O)[C@@H](CC(C)C)NC(=O)CCCC#Cc2cnc(*)nc2)cc1 ; *C(=O)OCc1ccc(NC(=O)[C@H](CCCCN)NC(=O)[C@H](CO)NC(=O)CNC(=O)CCCC#Cc2cnc(*)nc2)cc1 ; *C(=O)OCc1ccc(NC(=O)[C@H](C)NC(=O)[C@@H](NC(=O)CNC(=O)CNC(=O)CCCC#Cc2cnc(*)nc2)C(C)C)cc1 ; *C(=O)[C@H](C)NC(=O)[C@H](C)NC(=O)CCCC#Cc1cnc(*)nc1 ; *CNC(=O)[C@H](C)NC(=O)[C@H](C)NC(=O)CCCC#Cc1cnc(*)nc1]

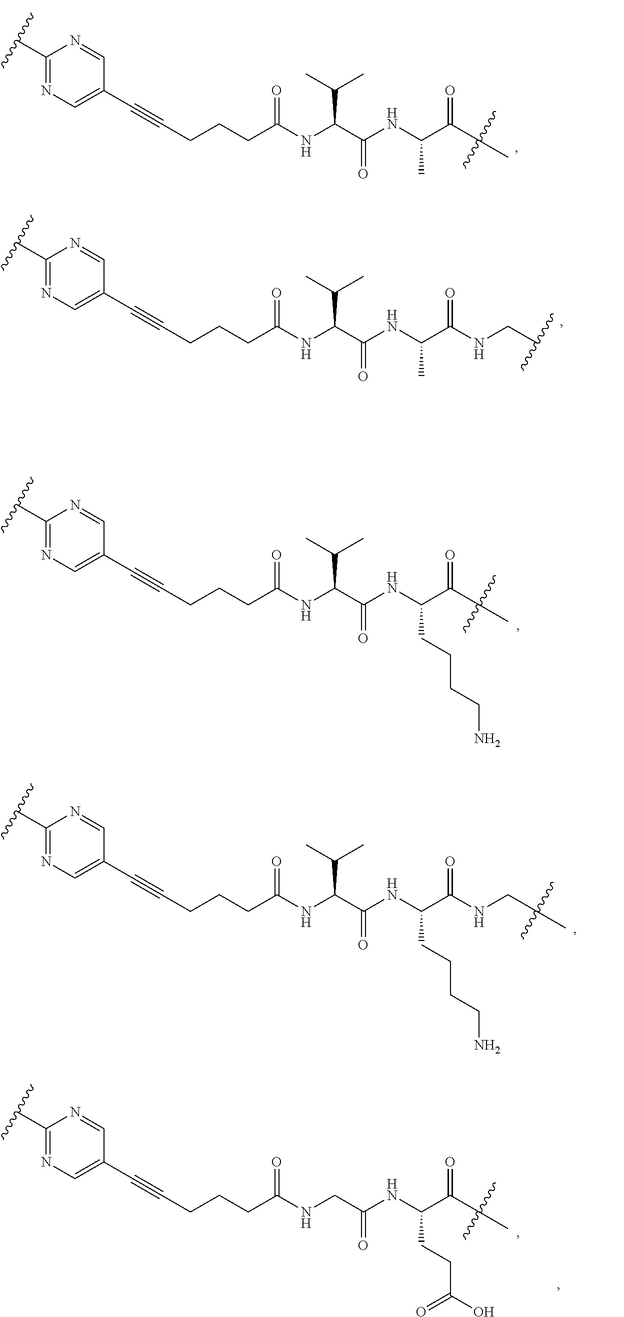 [SMILES: *C(=O)[C@H](C)NC(=O)[C@@H](NC(=O)CCCC#Cc1cnc(*)nc1)C(C)C ; *CNC(=O)[C@H](C)NC(=O)[C@@H](NC(=O)CCCC#Cc1cnc(*)nc1)C(C)C ; *C(=O)[C@H](CCCCN)NC(=O)[C@@H](NC(=O)CCCC#Cc1cnc(*)nc1)C(C)C ; *CNC(=O)[C@H](CCCCN)NC(=O)[C@@H](NC(=O)CCCC#Cc1cnc(*)nc1)C(C)C ; *C(=O)[C@H](CCC(=O)O)NC(=O)CNC(=O)CCCC#Cc1cnc(*)nc1]

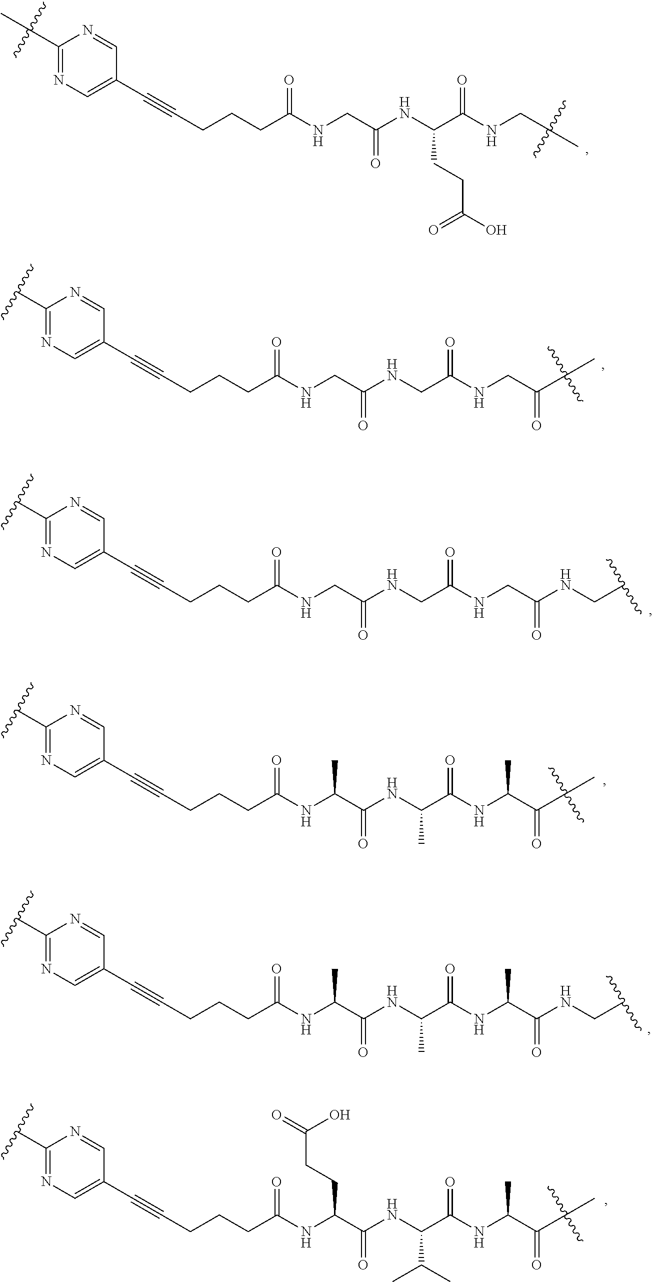 [SMILES: *CNC(=O)[C@H](CCC(=O)O)NC(=O)CNC(=O)CCCC#Cc1cnc(*)nc1 ; *C(=O)CNC(=O)CNC(=O)CNC(=O)CCCC#Cc1cnc(*)nc1 ; *CNC(=O)CNC(=O)CNC(=O)CNC(=O)CCCC#Cc1cnc(*)nc1 ; *C(=O)[C@H](C)NC(=O)[C@H](C)NC(=O)[C@H](C)NC(=O)CCCC#Cc1cnc(*)nc1 ; *CNC(=O)[C@H](C)NC(=O)[C@H](C)NC(=O)[C@H](C)NC(=O)CCCC#Cc1cnc(*)nc1 ; *C(=O)[C@H](C)NC(=O)[C@@H](NC(=O)[C@H](CCC(=O)O)NC(=O)CCCC#Cc1cnc(*)nc1)C(C)C]

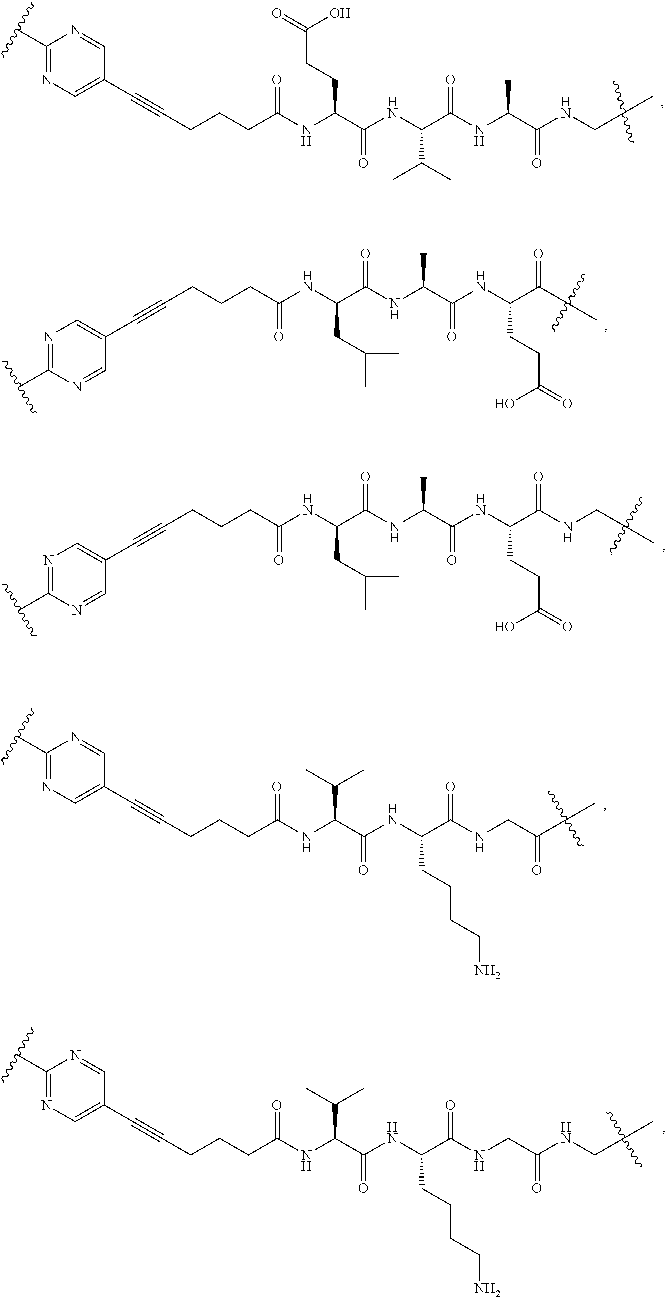 [SMILES: *CNC(=O)[C@H](C)NC(=O)[C@@H](NC(=O)[C@H](CCC(=O)O)NC(=O)CCCC#Cc1cnc(*)nc1)C(C)C ; *C(=O)[C@H](CCC(=O)O)NC(=O)[C@H](C)NC(=O)[C@@H](CC(C)C)NC(=O)CCCC#Cc1cnc(*)nc1 ; *CNC(=O)[C@H](CCC(=O)O)NC(=O)[C@H](C)NC(=O)[C@@H](CC(C)C)NC(=O)CCCC#Cc1cnc(*)nc1 ; *C(=O)CNC(=O)[C@H](CCCCN)NC(=O)[C@@H](NC(=O)CCCC#Cc1cnc(*)nc1)C(C)C ; *CNC(=O)CNC(=O)[C@H](CCCCN)NC(=O)[C@@H](NC(=O)CCCC#Cc1cnc(*)nc1)C(C)C]

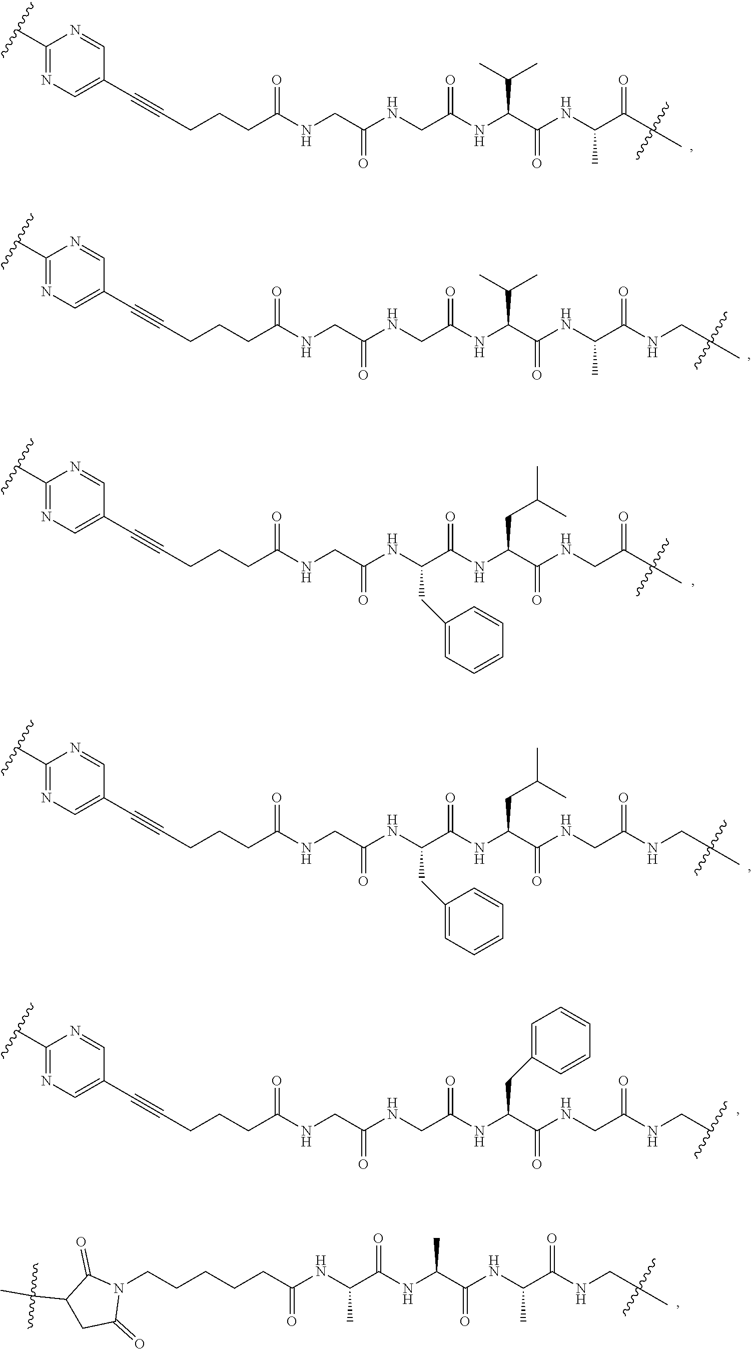 [SMILES: *C(=O)[C@H](C)NC(=O)[C@@H](NC(=O)CNC(=O)CNC(=O)CCCC#Cc1cnc(*)nc1)C(C)C ; *CNC(=O)[C@H](C)NC(=O)[C@@H](NC(=O)CNC(=O)CNC(=O)CCCC#Cc1cnc(*)nc1)C(C)C ; *C(=O)CNC(=O)[C@H](CC(C)C)NC(=O)[C@H](Cc1ccccc1)NC(=O)CNC(=O)CCCC#Cc1cnc(*)nc1 ; *CNC(=O)CNC(=O)[C@H](CC(C)C)NC(=O)[C@H](Cc1ccccc1)NC(=O)CNC(=O)CCCC#Cc1cnc(*)nc1 ; *CNC(=O)CNC(=O)[C@H](Cc1ccccc1)NC(=O)CNC(=O)CNC(=O)CCCC#Cc1cnc(*)nc1 ; *CNC(=O)[C@H](C)NC(=O)[C@H](C)NC(=O)[C@H](C)NC(=O)CCCCCN1C(=O)CC(*)C1=O]

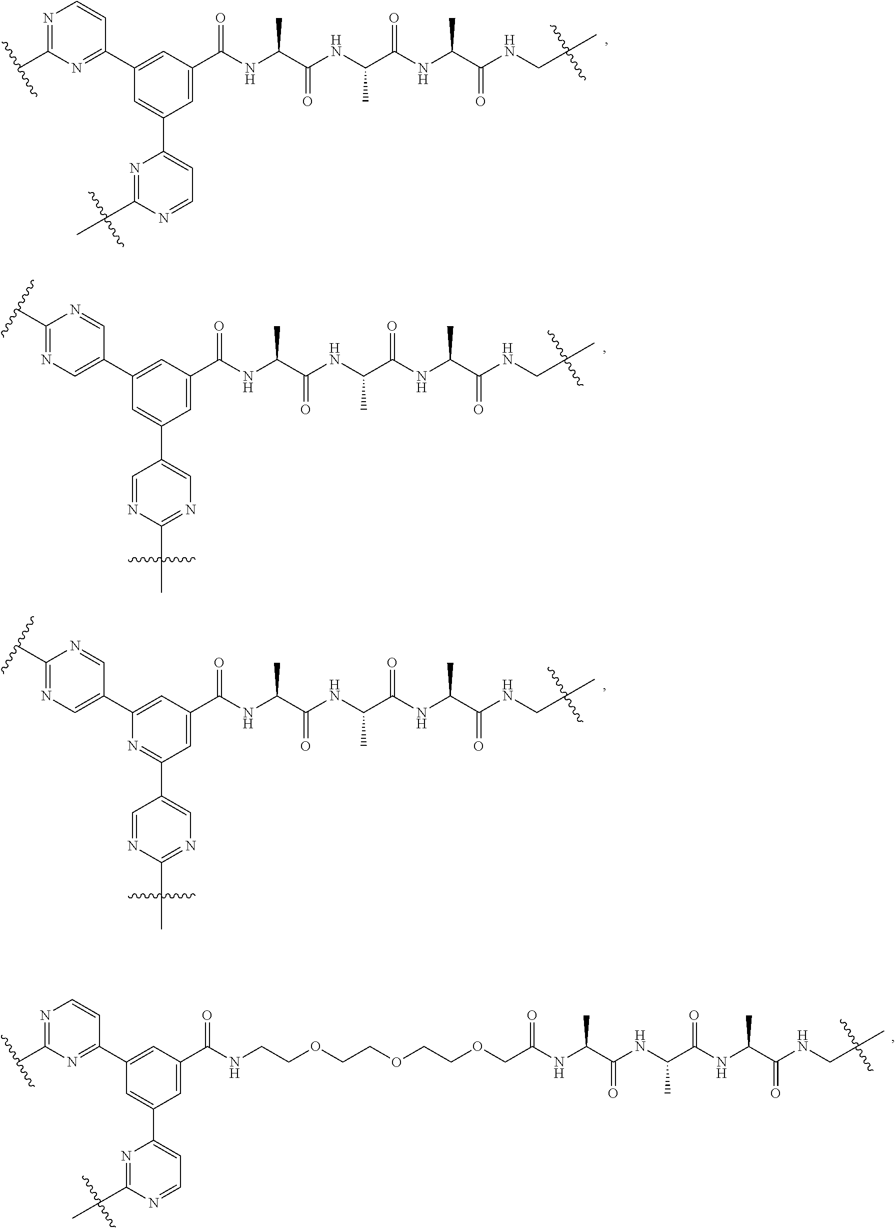 [SMILES: *CNC(=O)[C@H](C)NC(=O)[C@H](C)NC(=O)[C@H](C)NC(=O)c1cc(-c2ccnc(*)n2)cc(-c2ccnc(*)n2)c1 ; *CNC(=O)[C@H](C)NC(=O)[C@H](C)NC(=O)[C@H](C)NC(=O)c1cc(-c2cnc(*)nc2)cc(-c2cnc(*)nc2)c1 ; *CNC(=O)[C@H](C)NC(=O)[C@H](C)NC(=O)[C@H](C)NC(=O)c1cc(-c2cnc(*)nc2)nc(-c2cnc(*)nc2)c1 ; *CNC(=O)[C@H](C)NC(=O)[C@H](C)NC(=O)[C@H](C)NC(=O)COCCOCCOCCNC(=O)c1cc(-c2ccnc(*)n2)cc(-c2ccnc(*)n2)c1]

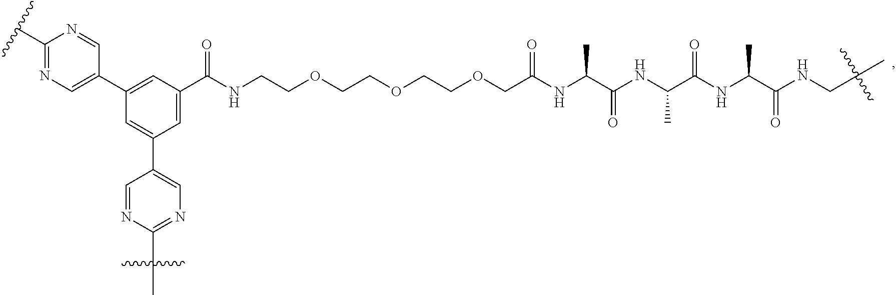 [SMILES: *CNC(=O)[C@H](C)NC(=O)[C@H](C)NC(=O)[C@H](C)NC(=O)COCCOCCOCCNC(=O)c1cc(-c2cnc(*)nc2)cc(-c2cnc(*)nc2)c1]

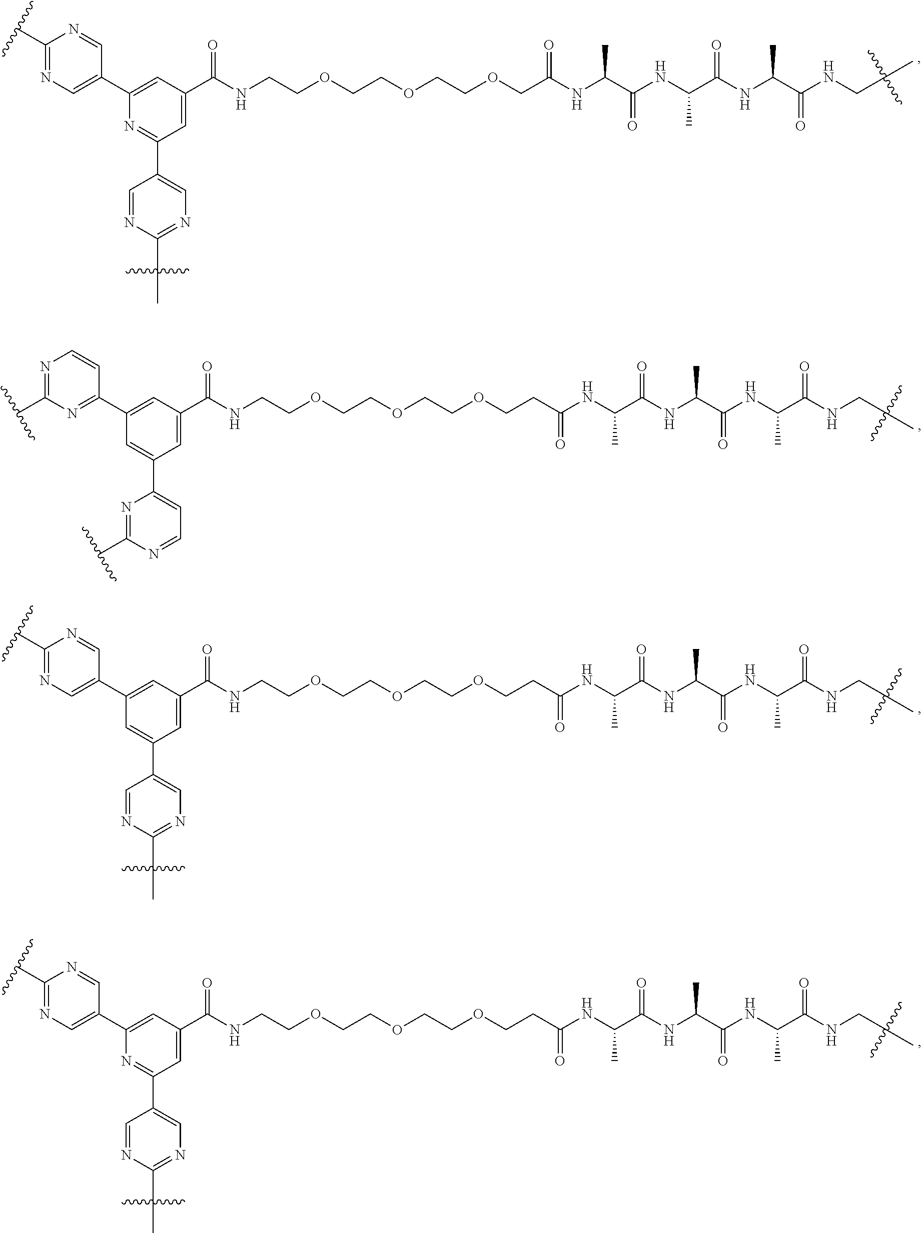 [SMILES: *CNC(=O)[C@H](C)NC(=O)[C@H](C)NC(=O)[C@H](C)NC(=O)COCCOCCOCCNC(=O)c1cc(-c2cnc(*)nc2)nc(-c2cnc(*)nc2)c1 ; *CNC(=O)[C@H](C)NC(=O)[C@H](C)NC(=O)[C@H](C)NC(=O)CCOCCOCCOCCNC(=O)c1cc(-c2ccnc(*)n2)cc(-c2ccnc(*)n2)c1 ; *CNC(=O)[C@H](C)NC(=O)[C@H](C)NC(=O)[C@H](C)NC(=O)CCOCCOCCOCCNC(=O)c1cc(-c2cnc(*)nc2)cc(-c2cnc(*)nc2)c1 ; *CNC(=O)[C@H](C)NC(=O)[C@H](C)NC(=O)[C@H](C)NC(=O)CCOCCOCCOCCNC(=O)c1cc(-c2cnc(*)nc2)nc(-c2cnc(*)nc2)c1]

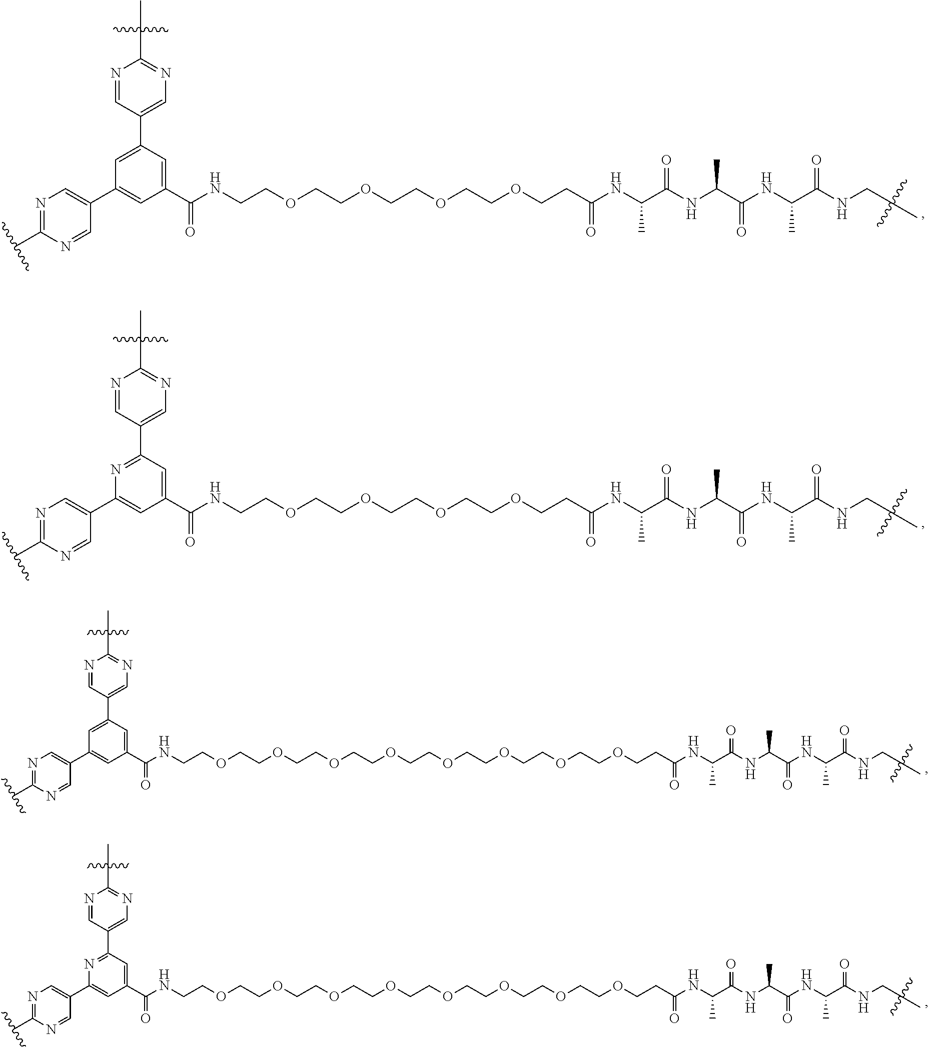 [SMILES: *CNC(=O)[C@H](C)NC(=O)[C@H](C)NC(=O)[C@H](C)NC(=O)CCOCCOCCOCCOCCNC(=O)c1cc(-c2cnc(*)nc2)cc(-c2cnc(*)nc2)c1 ; *CNC(=O)[C@H](C)NC(=O)[C@H](C)NC(=O)[C@H](C)NC(=O)CCOCCOCCOCCOCCNC(=O)c1cc(-c2cnc(*)nc2)nc(-c2cnc(*)nc2)c1 ; *CNC(=O)[C@H](C)NC(=O)[C@H](C)NC(=O)[C@H](C)NC(=O)CCOCCOCCOCCOCCOCCOCCOCCOCCNC(=O)c1cc(-c2cnc(*)nc2)cc(-c2cnc(*)nc2)c1 ; *CNC(=O)[C@H](C)NC(=O)[C@H](C)NC(=O)[C@H](C)NC(=O)CCOCCOCCOCCOCCOCCOCCOCCOCCNC(=O)c1cc(-c2cnc(*)nc2)nc(-c2cnc(*)nc2)c1]

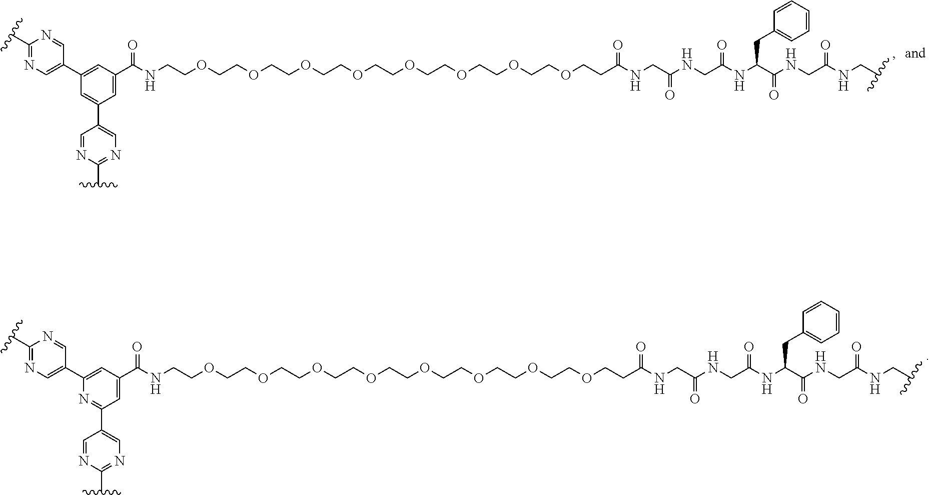 [SMILES: *CNC(=O)CNC(=O)[C@H](Cc1ccccc1)NC(=O)CNC(=O)CNC(=O)CCOCCOCCOCCOCCOCCOCCOCCOCCNC(=O)c1cc(-c2cnc(*)nc2)cc(-c2cnc(*)nc2)c1 ; *CNC(=O)CNC(=O)[C@H](Cc1ccccc1)NC(=O)CNC(=O)CNC(=O)CCOCCOCCOCCOCCOCCOCCOCCOCCNC(=O)c1cc(-c2cnc(*)nc2)nc(-c2cnc(*)nc2)c1]

Embodiment 56. The antibody-drug conjugate of described in any one of embodiments 1-54, wherein

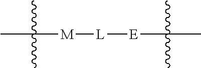

is selected from the following structures:

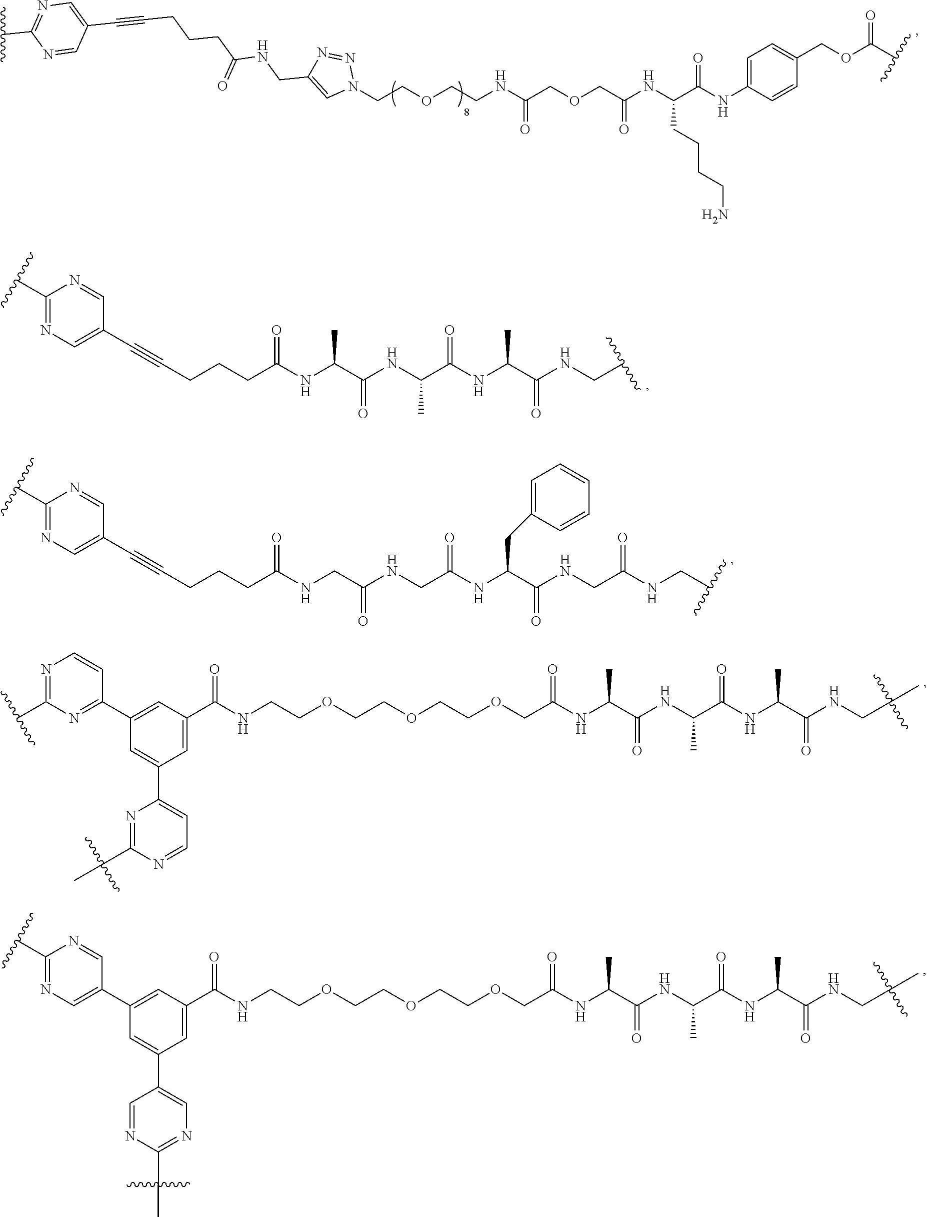

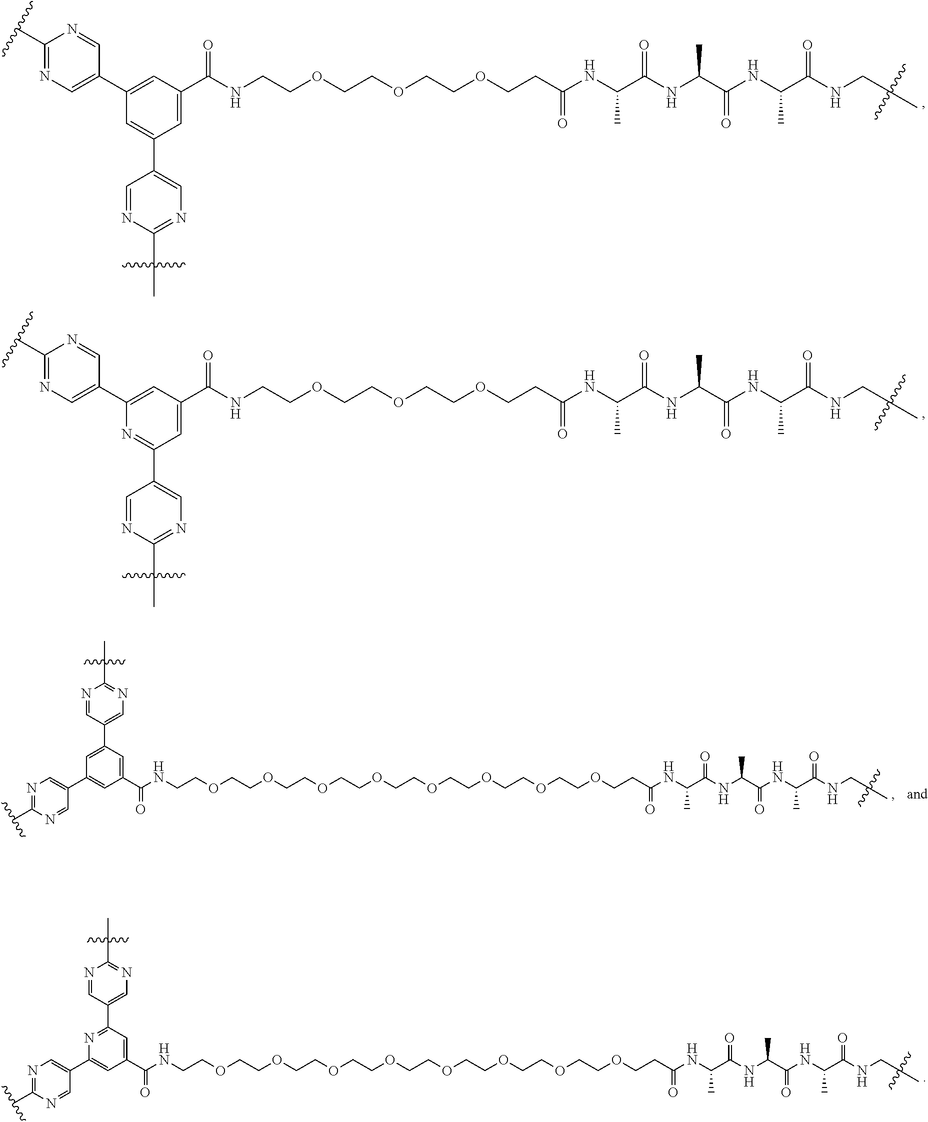 [SMILES: *CNC(=O)[C@H](C)NC(=O)[C@H](C)NC(=O)[C@H](C)NC(=O)CCOCCOCCOCCNC(=O)c1cc(-c2cnc(*)nc2)cc(-c2cnc(*)nc2)c1 ; *CNC(=O)[C@H](C)NC(=O)[C@H](C)NC(=O)[C@H](C)NC(=O)CCOCCOCCOCCNC(=O)c1cc(-c2cnc(*)nc2)nc(-c2cnc(*)nc2)c1 ; *CNC(=O)[C@H](C)NC(=O)[C@H](C)NC(=O)[C@H](C)NC(=O)CCOCCOCCOCCOCCOCCOCCOCCOCCNC(=O)c1cc(-c2cnc(*)nc2)cc(-c2cnc(*)nc2)c1 ; *CNC(=O)[C@H](C)NC(=O)[C@H](C)NC(=O)[C@H](C)NC(=O)CCOCCOCCOCCOCCOCCOCCOCCOCCNC(=O)c1cc(-c2cnc(*)nc2)nc(-c2cnc(*)nc2)c1]

Embodiment 57. The antibody-drug conjugate of any one of embodiments 1-56, wherein the cytotoxic drug is selected from the group consisting of tubulin inhibitors, DNA intercalating agents, DNA Topoisomerase inhibitors, and RNA polymerase inhibitors.

Embodiment 58. The antibody-drug conjugate of embodiment 57, wherein the tubulin inhibitor is an auristatin compound or a maytansinoid compound.

Embodiment 59. The antibody-drug conjugate of embodiment 57, wherein the DNA intercalator is pyrrolobenzodiazepine (PBD).

Embodiment 60. The antibody-drug conjugate of embodiment 57, wherein the DNA topoisomerase inhibitor is a topoisomerase I inhibitor or a topoisomerase II inhibitor.

Embodiment 61. The antibody-drug conjugate of embodiment 60, wherein the topoisomerase I inhibitor is selected from camptothecin, hydroxycamptothecin, 9-aminocamptothecin, SN-38, irinotecan, topotecan, bellotecan, rubotecan, and a pharmaceutically acceptable salt, ester, or analog thereof, and the topoisomerase II inhibitor is selected from doxorubicin, PNU-159682, docarmicin, daunorubicin, mitoxantrone, podophyllin toxin, etoposide, and a pharmaceutically acceptable salt, ester, or analog thereof.

Embodiment 62. The antibody-drug conjugate of embodiment 57, wherein the RNA polymerase inhibitor is α-amanitin or a pharmaceutically acceptable salt, ester, or analog thereof.

Embodiment 63. The antibody-drug conjugate of any one of embodiments 1-56, wherein the cytotoxic drug is selected from the group consisting of compounds represented by Formula I and Formula II, or pharmaceutically acceptable salts, esters, stereoisomers, tautomers or precursors of the compounds represented by Formula I and Formula II:

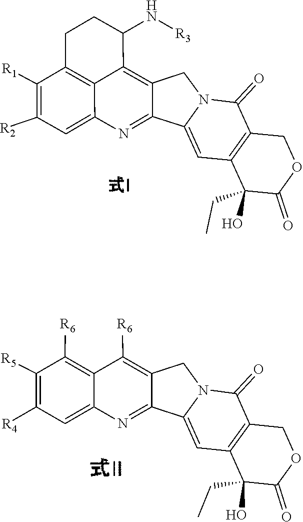

wherein R₁ and R₂ are each independently selected from C₁₋₆ Alkyl and halogen; R₃ is selected from H and —CO—CH₂OH; R₄ and R₅ are each independently selected from H, halogen, and hydroxyl, or R₄ and R₅ are connected to form a 5-6 membered oxygen-containing heterocyclic ring; R₆ is selected from hydrogen or —C₁₋₄ alkylene-NRₐR_{b}; R₇ is selected from C₁₋₆ alkyl, —C₁₋₄ alkylene-NRₐR_{b}, —C₁₋₄ alkylene-SiRₐR_{b}R_{c}, —SiRₐR_{b}R_{c}, —C₁₋₄ alkylene=N—ORₐ; wherein, Rₐ, R_{b} and R_{c} are independently selected from H, C₁₋₆ alkyl, —SO₂—C₁₋₆ alkyl, and —CO—C₁₋₆ alkyl at each occurrence; wherein optionally Rₐ and R_{b} connected to the associated atoms form a 5-6 membered nitrogen containing heterocyclic ring.

Embodiment 64. The antibody-drug conjugate of any one of embodiments 1-56, wherein the cytotoxic drug is selected from the following compounds or pharmaceutically acceptable salts, esters, stereoisomers, tautomers, or prodrugs of said compounds:

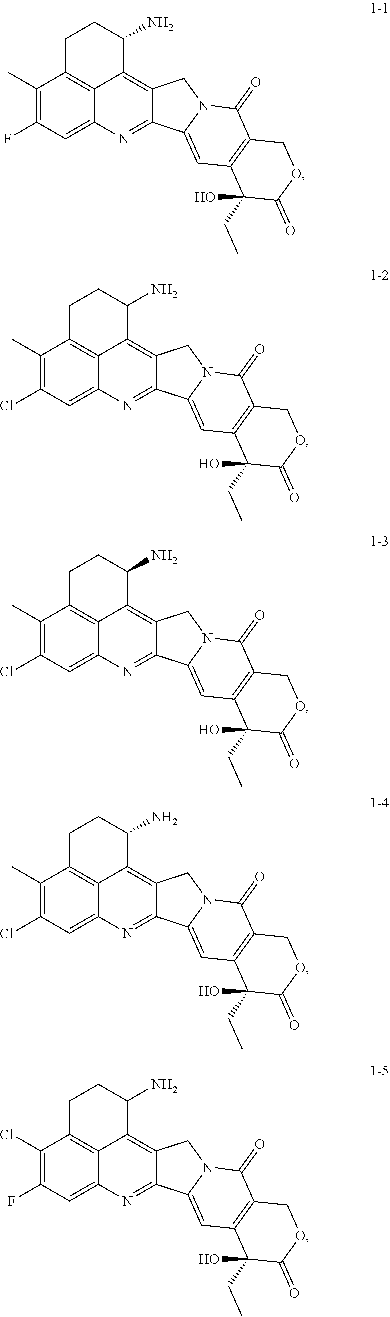 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2C(N)CC3]

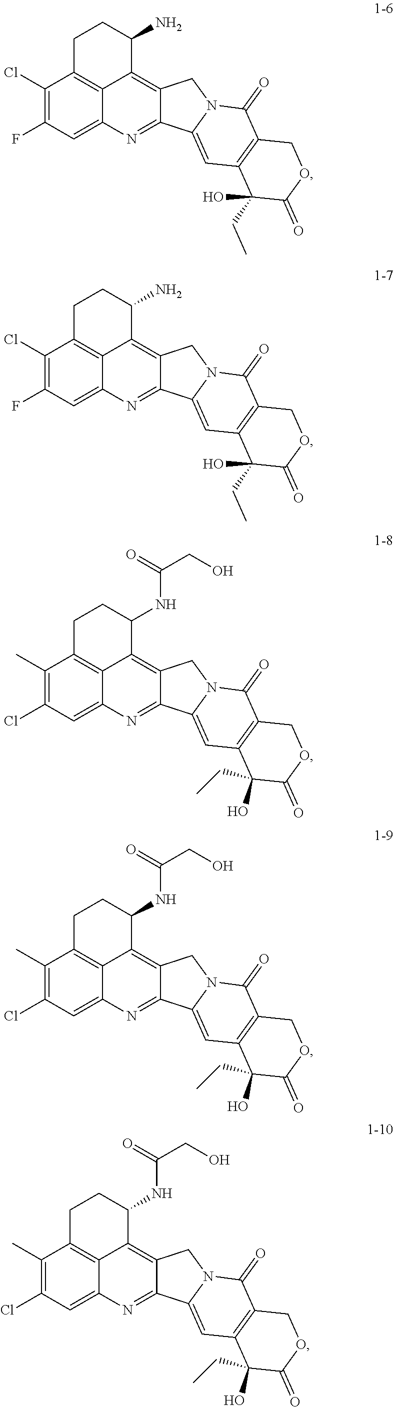 [SMILES: CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3]

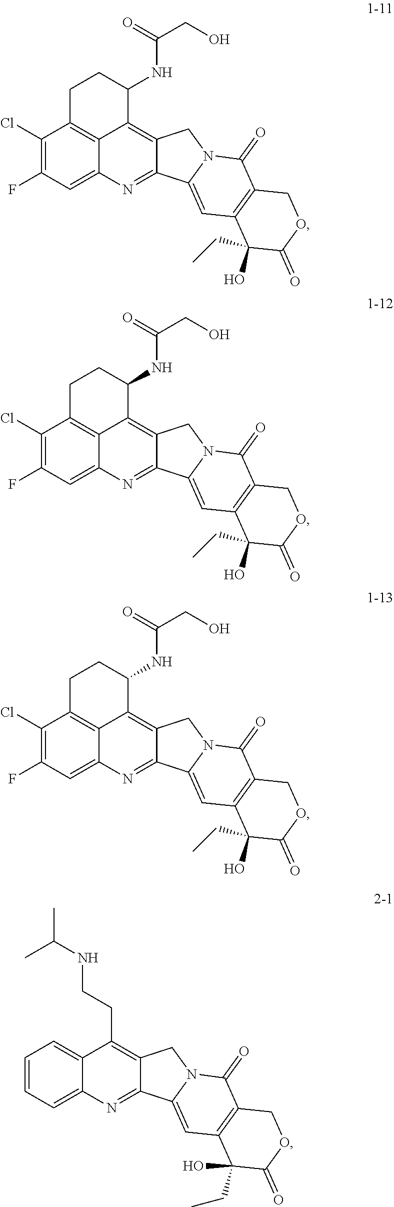 [SMILES: CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1ccccc1c2CCNC(C)C]

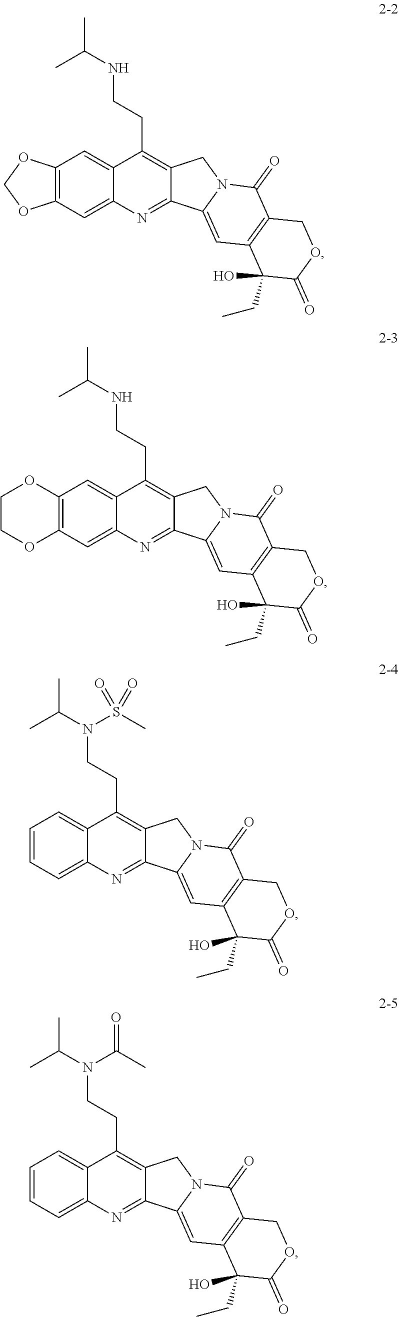 [SMILES: CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1cc3c(cc1c2CCNC(C)C)OCCO3 ; CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1ccccc1c2CCN(C(C)=O)C(C)C]

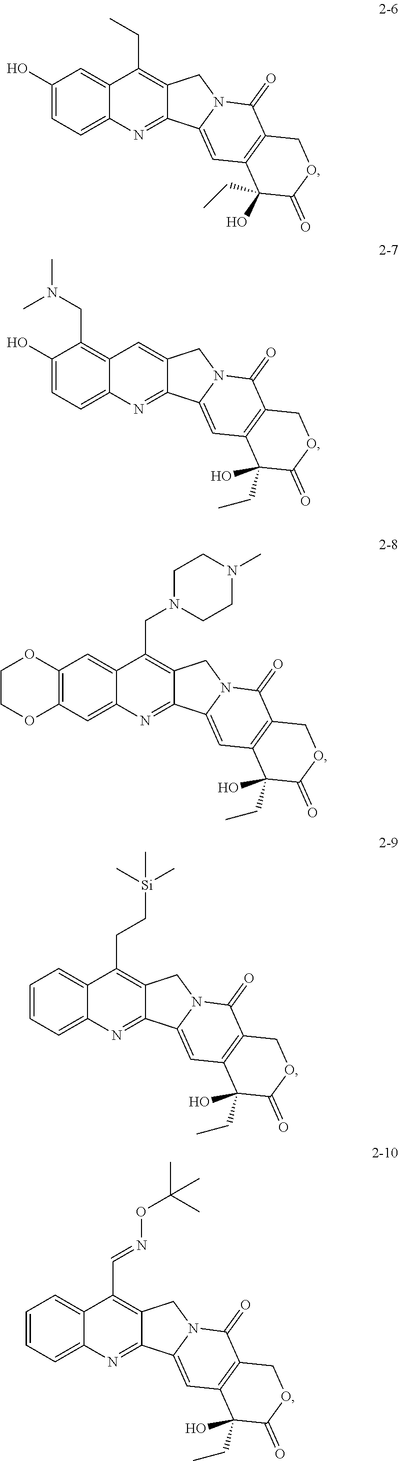

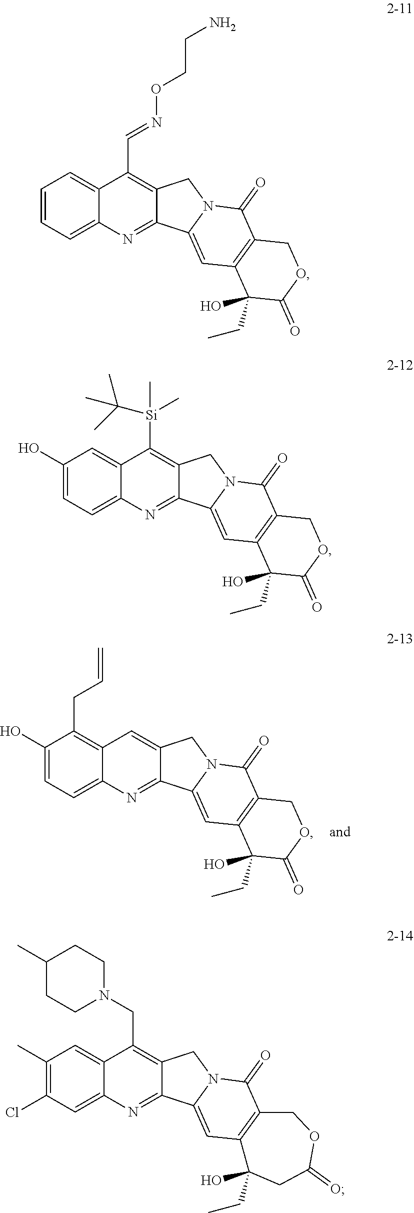 [SMILES: CC[C@@]1(O)C(=O)OCc2c1cc1n(c2=O)Cc2c-1nc1ccccc1c2/C=N/OCCN ; CC[C@@]1(O)C(=O)OCc2c1cc1n(c2=O)Cc2c-1nc1ccc(O)cc1c2[Si](C)(C)C(C)(C)C ; C=CCc1c(O)ccc2nc3c(cc12)Cn1c-3cc2c(c1=O)COC(=O)[C@]2(O)CC ; CC[C@@]1(O)CC(=O)OCc2c1cc1n(c2=O)Cc2c-1nc1cc(Cl)c(C)cc1c2CN1CCC(C)CC1]

wherein the corresponding residue of the cytotoxic drug obtained after connecting the cytotoxic drug to the linker is D in the formula of embodiment 1.

Embodiment 65. The antibody-drug conjugate of any one of embodiments 1-56, wherein the cytotoxic drug is selected from the following compounds or pharmaceutically acceptable salts, esters, stereoisomers, tautomers or prodrugs of said compounds:

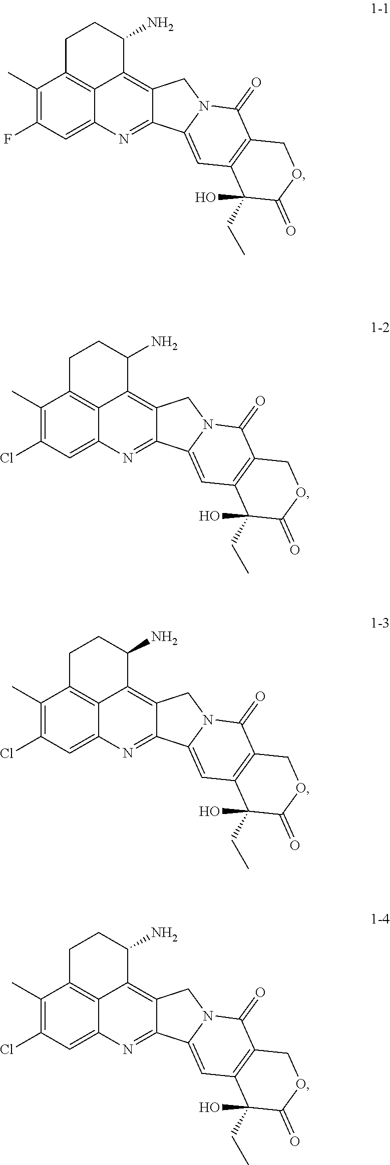 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3]

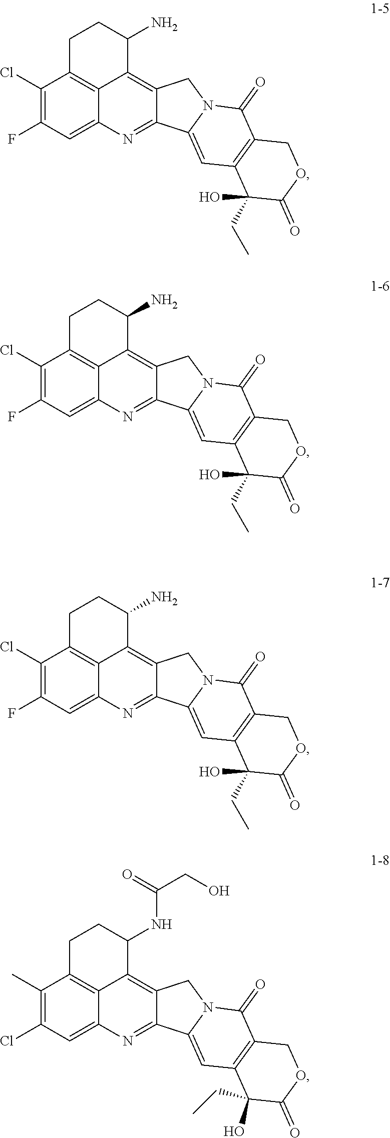 [SMILES: CC[C@@]1(O)C(=O)OCc2c1cc1n(c2=O)Cc2c-1nc1cc(F)c(Cl)c3c1c2C(N)CC3 ; CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3]

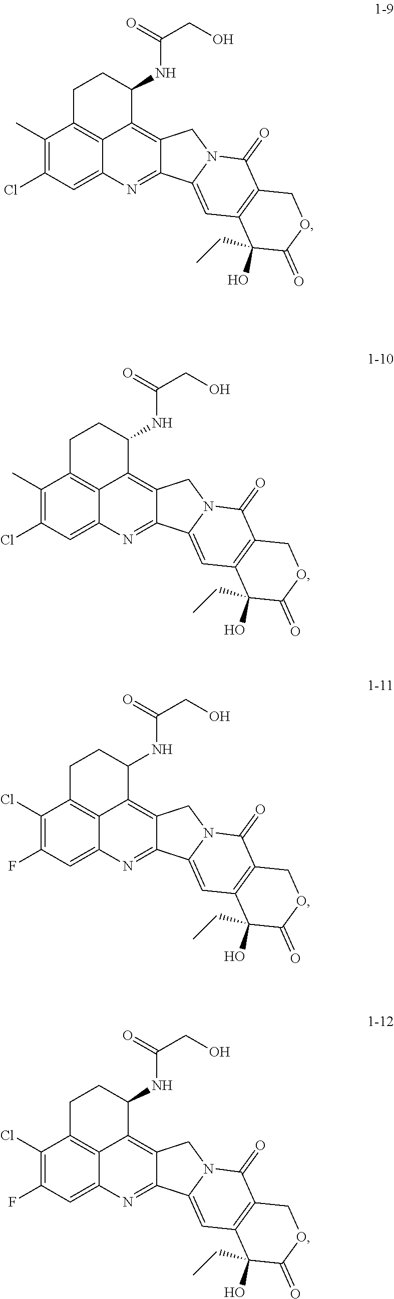 [SMILES: CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3]

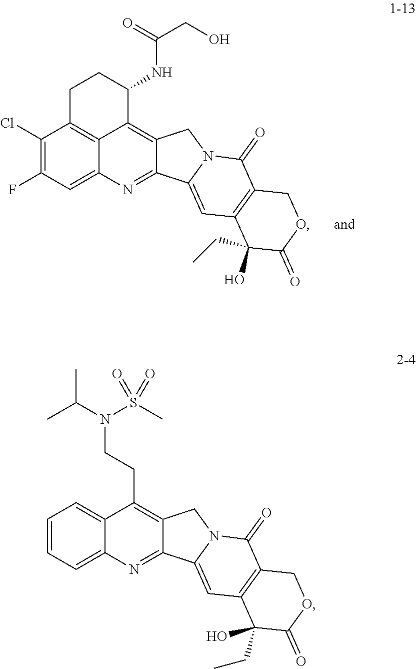 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1ccccc1c2CCN(C(C)C)S(C)(=O)=O]

wherein the corresponding residue of the cytotoxic drug obtained after connecting the cytotoxic drug to the linker is D in the formula of embodiment 1.

Embodiment 66. The antibody-drug conjugate of embodiment 64 or 65, wherein D is a monovalent structure obtained by the loss of one H from an —OH, an —NH₂, or a secondary amine on the cytotoxic drug.

Embodiment 67. The antibody-drug conjugate of any one of embodiments 1-66, wherein the antibody-conjugate is selected from:

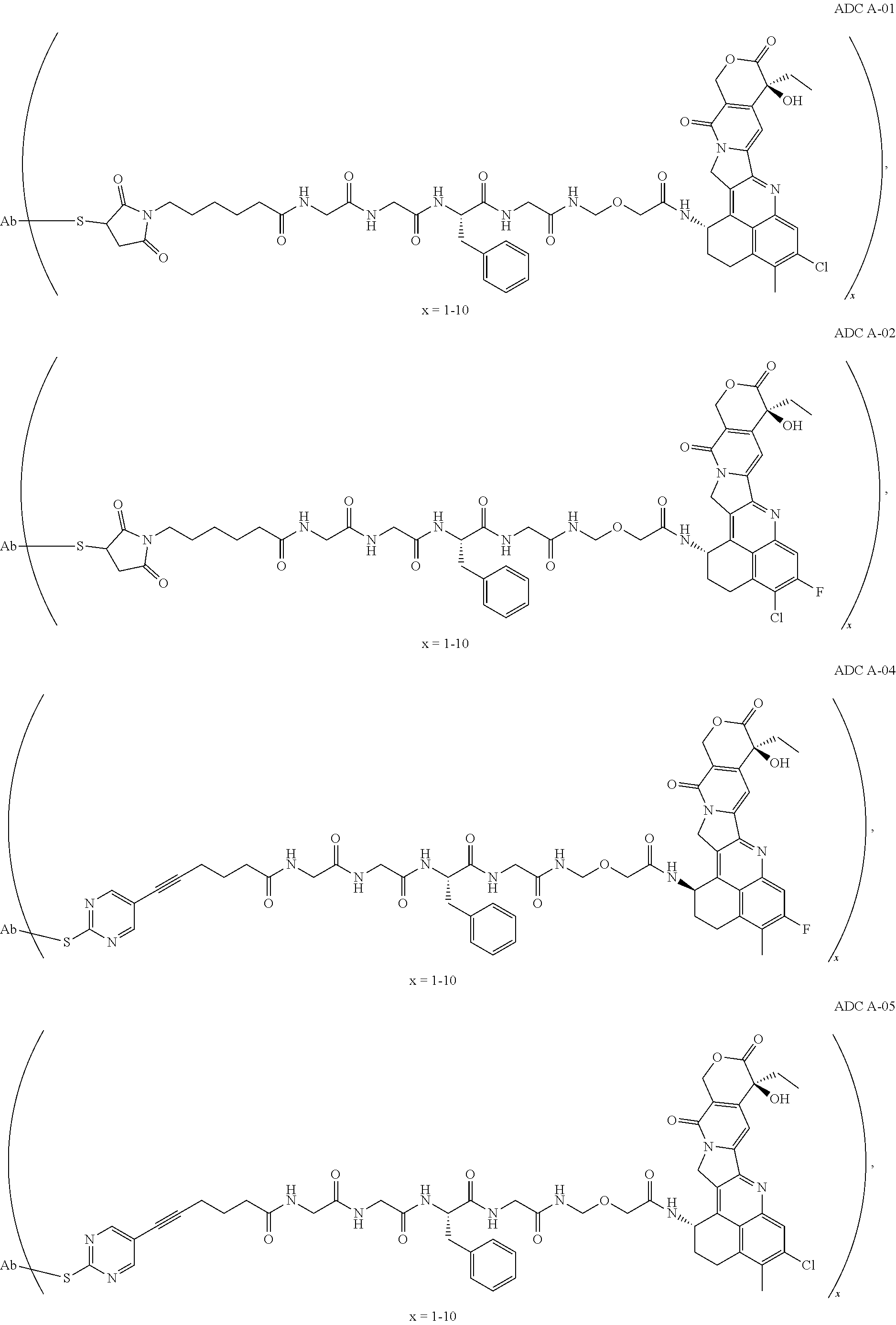

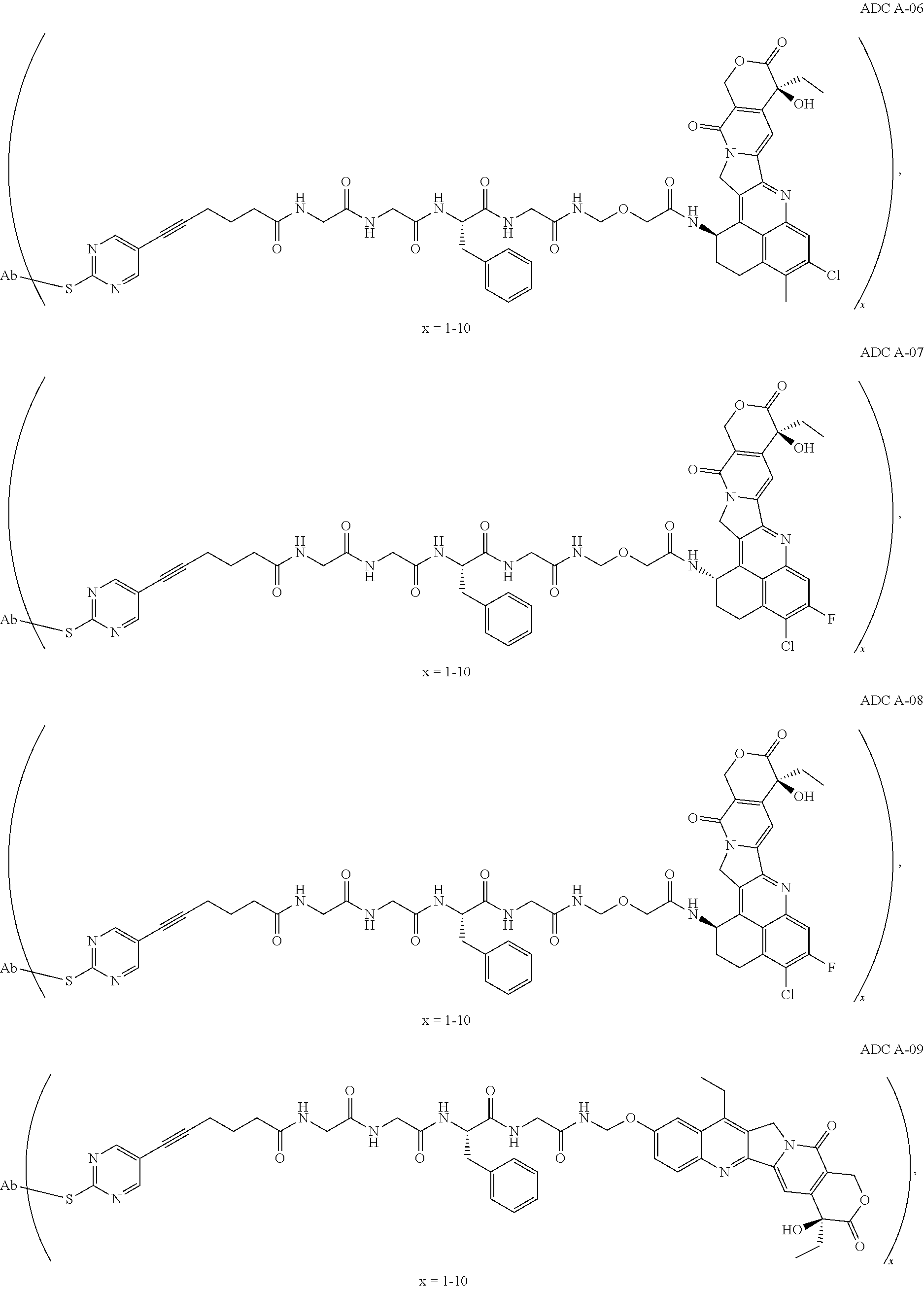

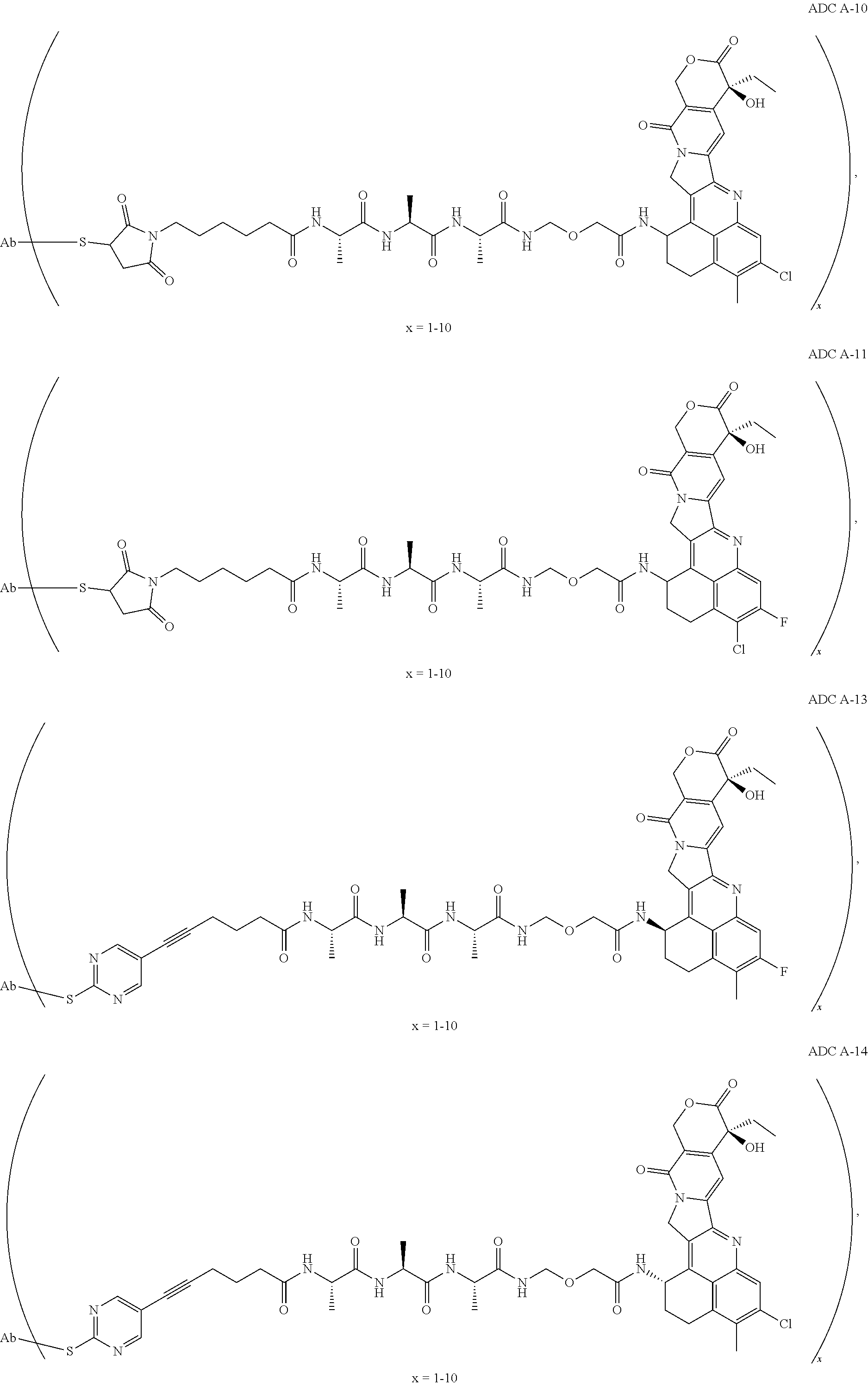

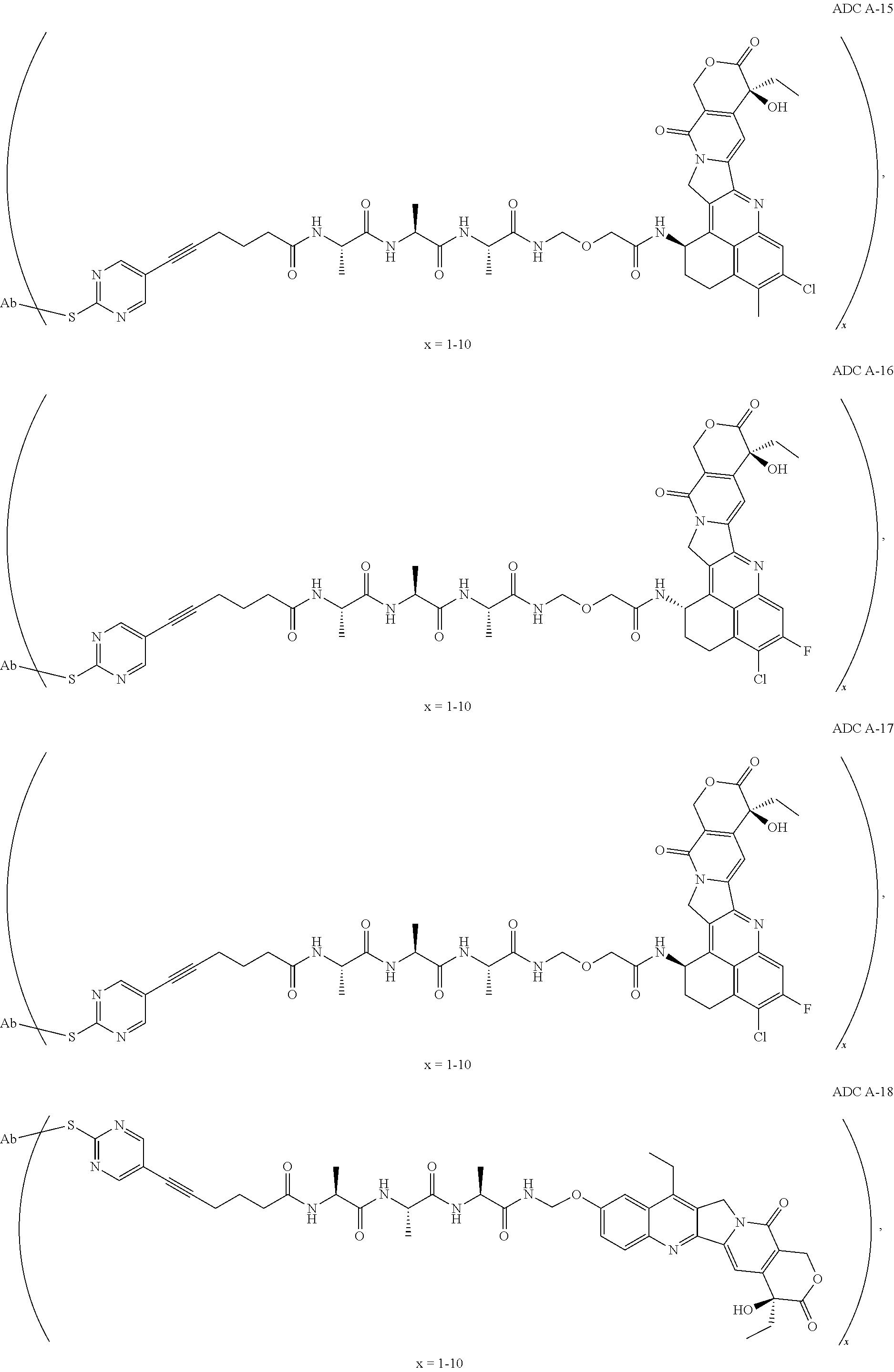

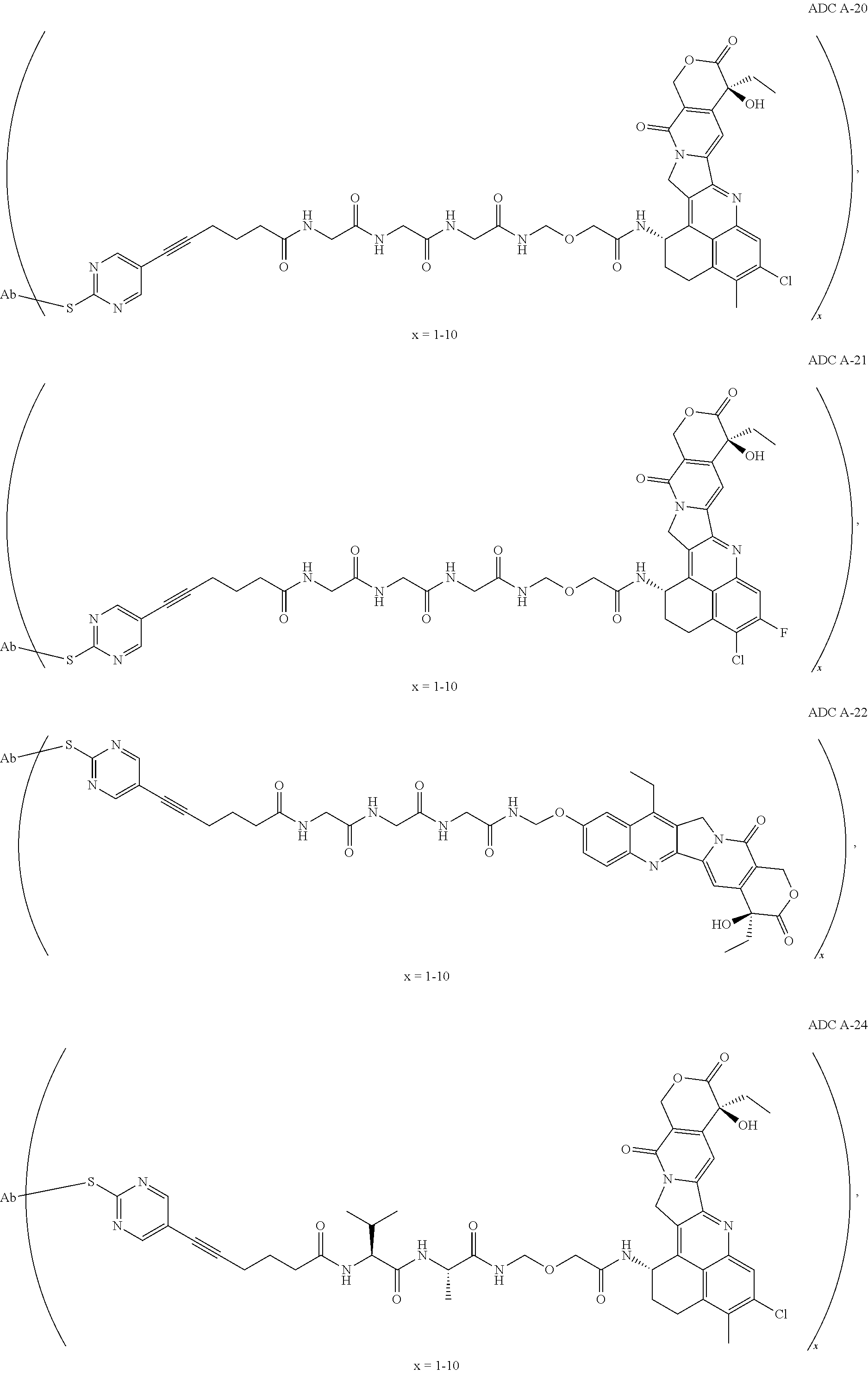

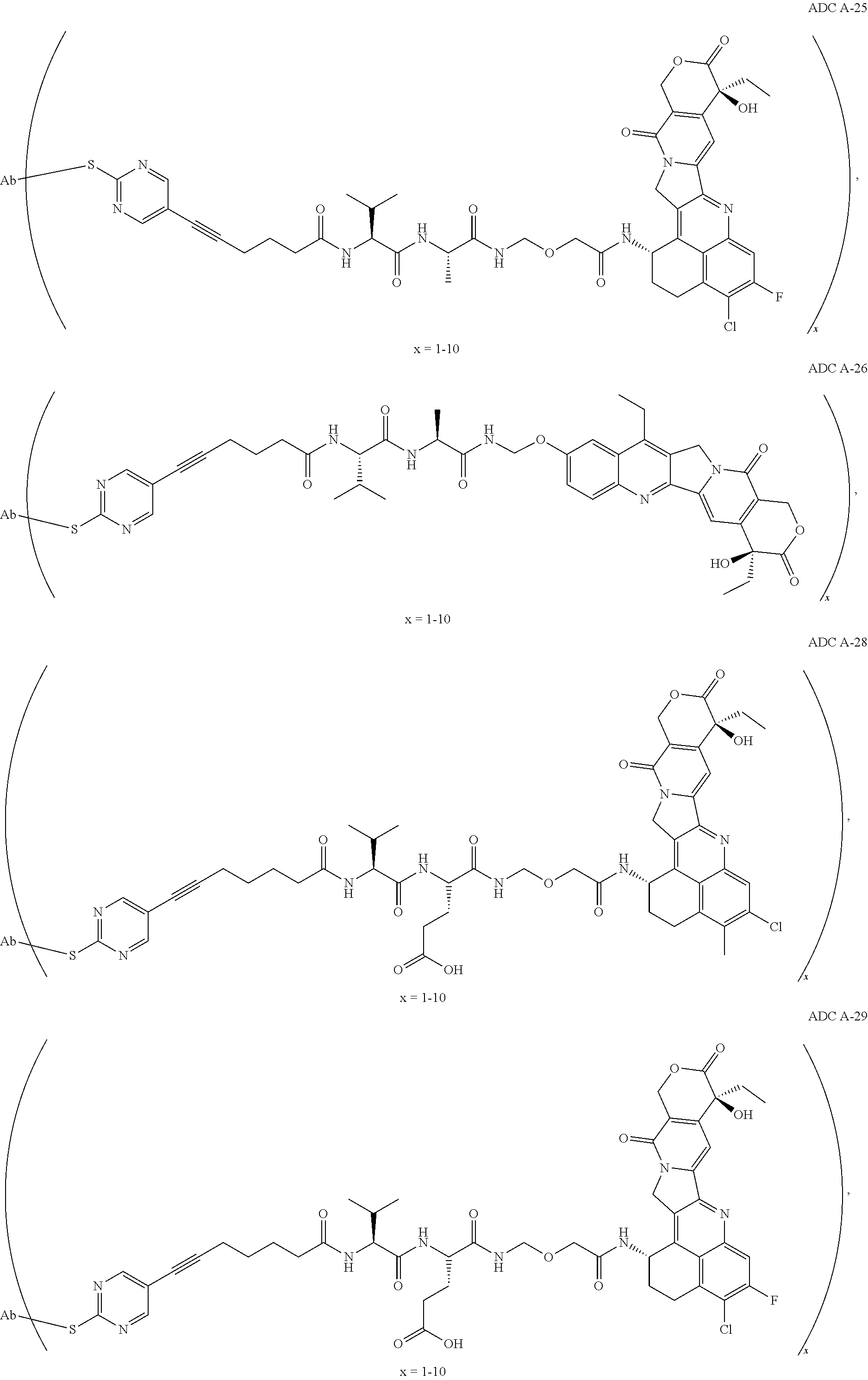

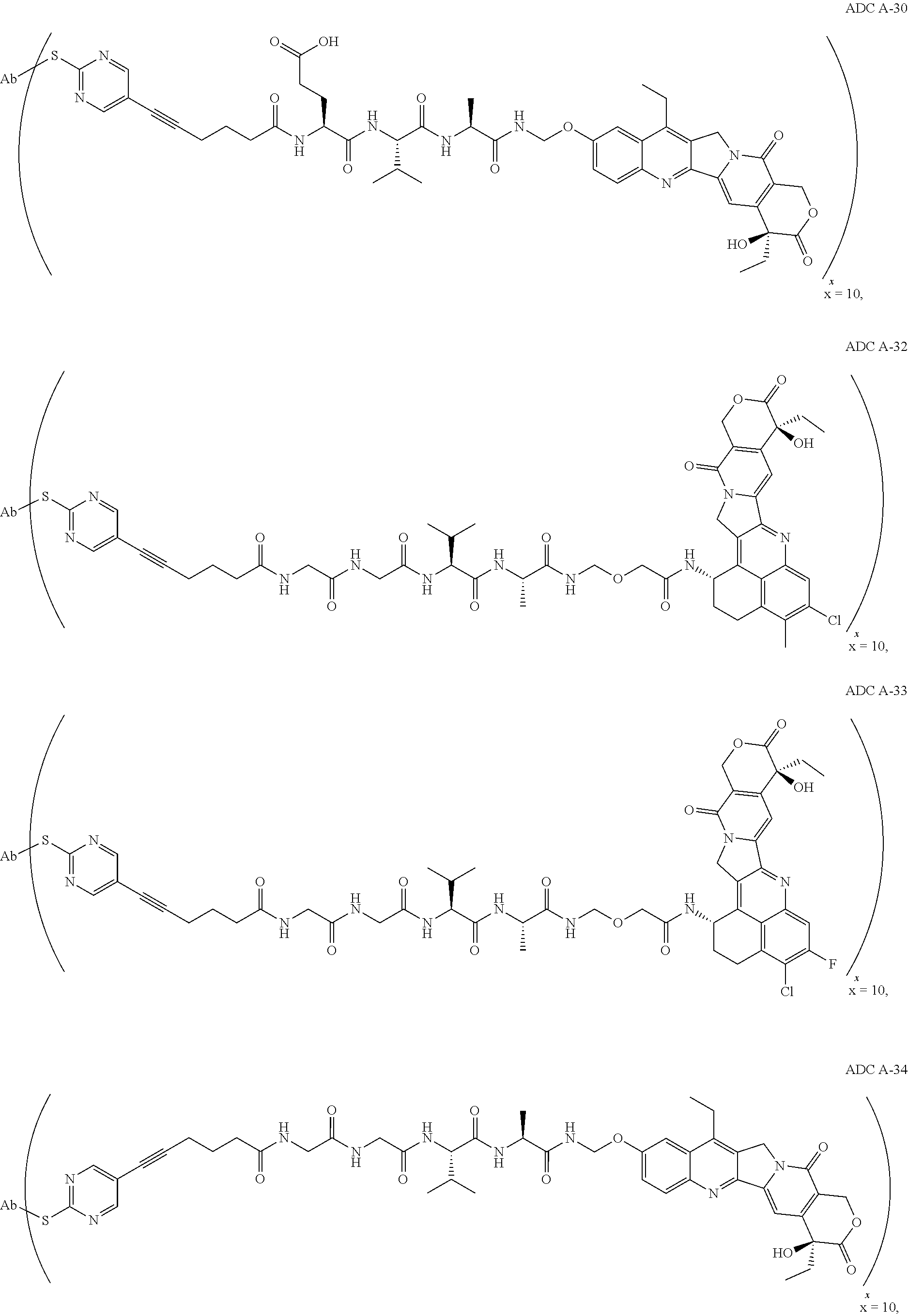

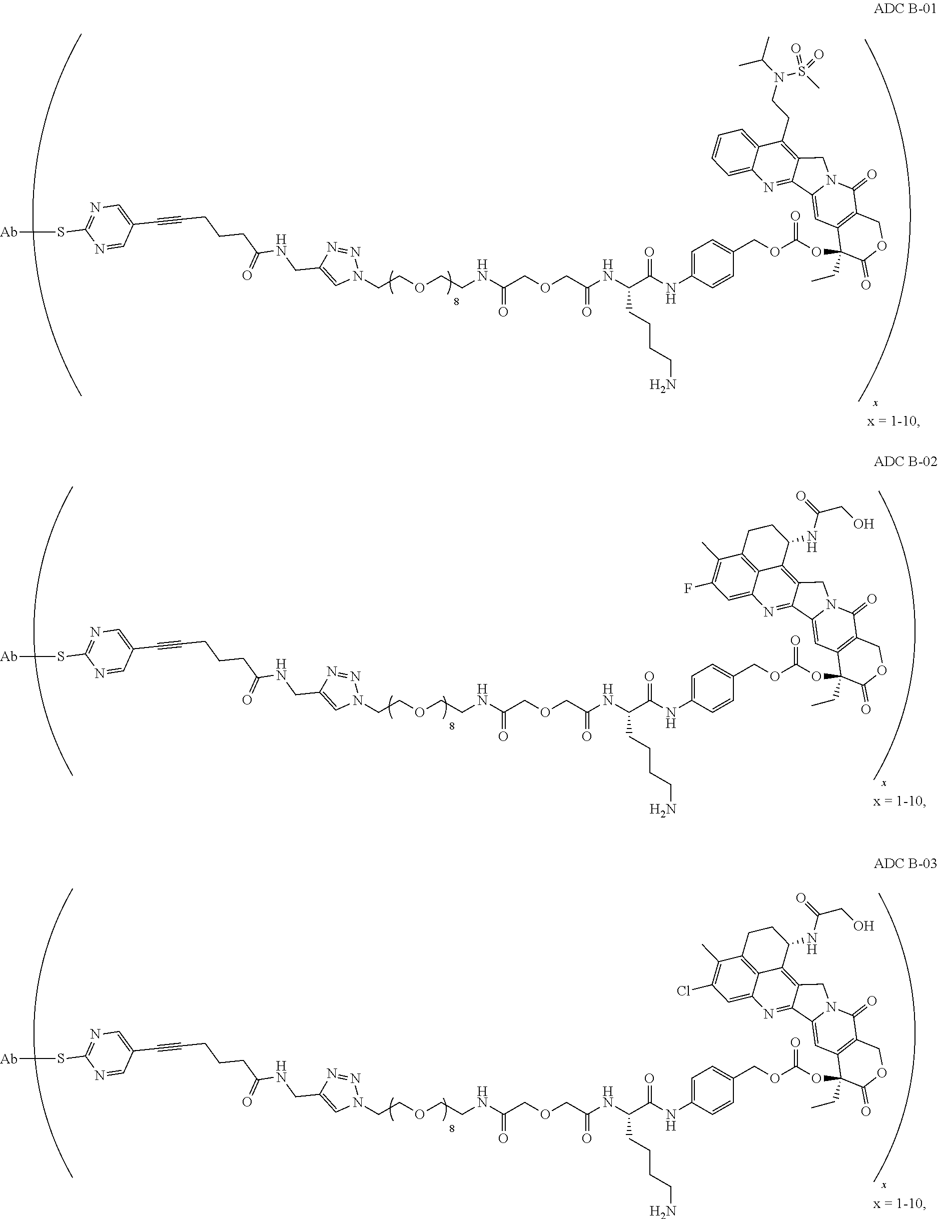

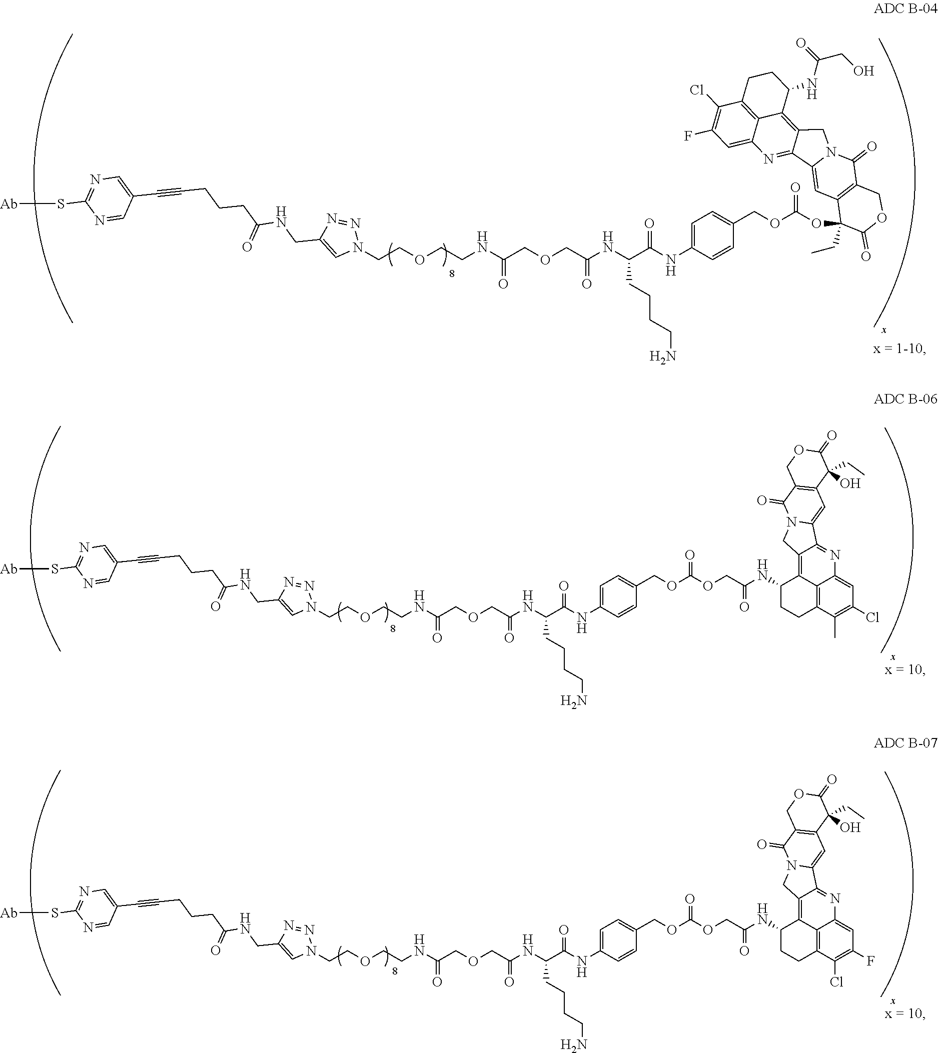

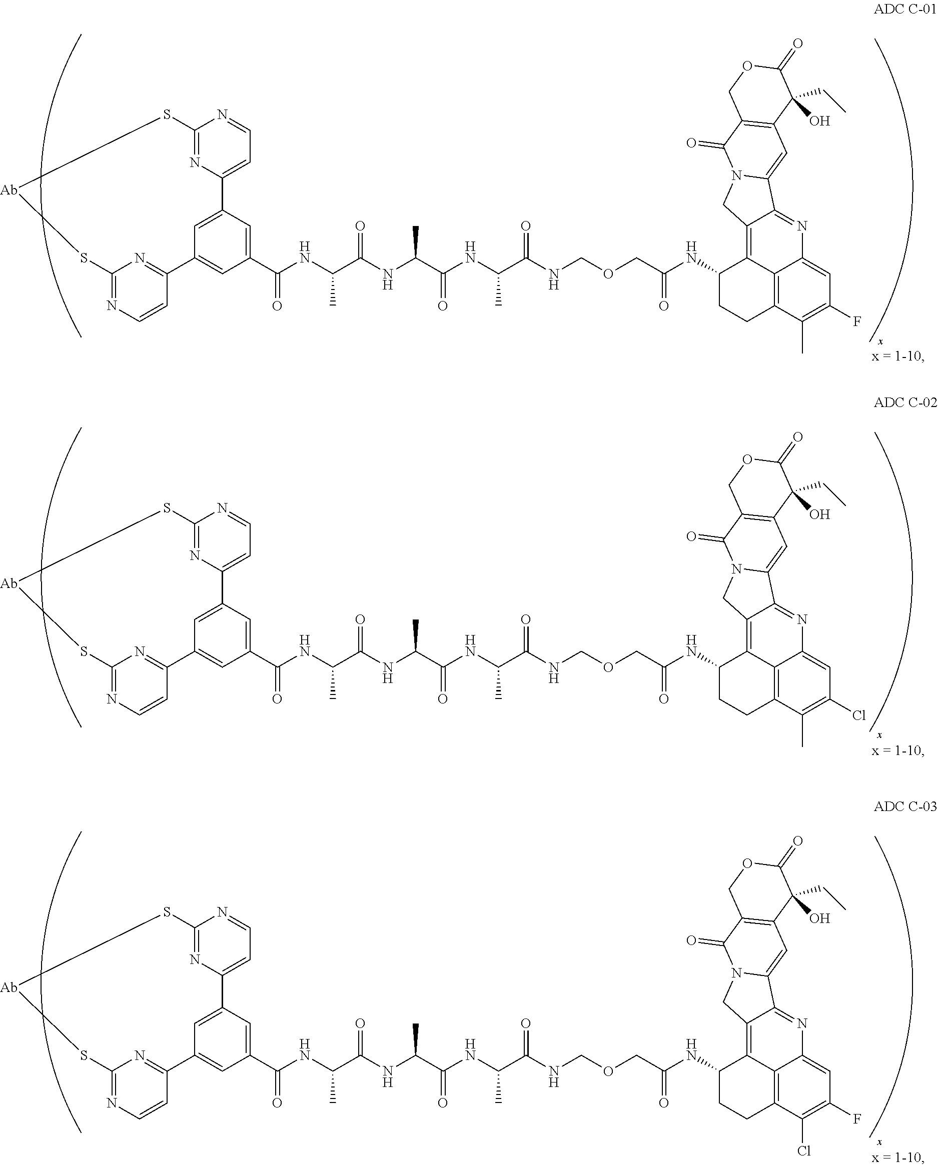

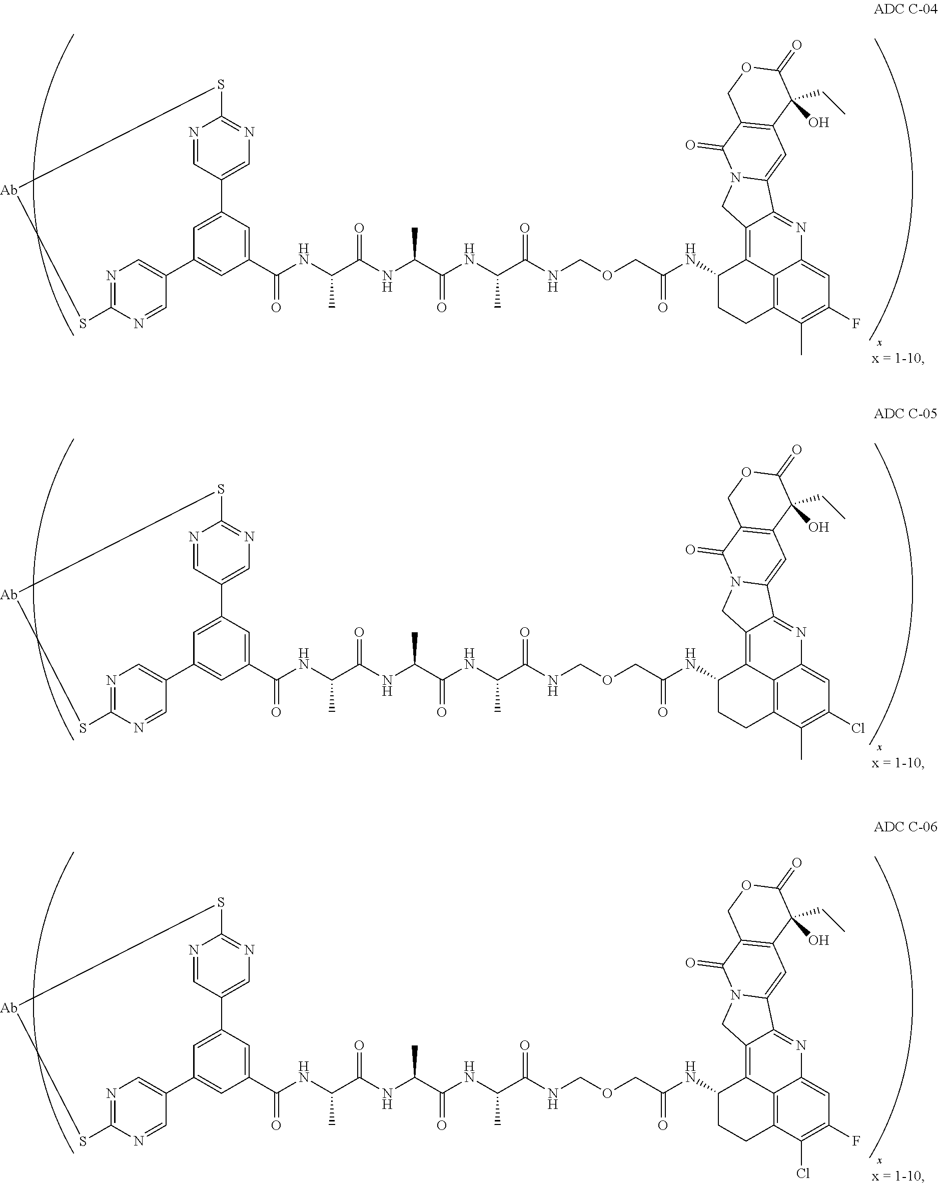

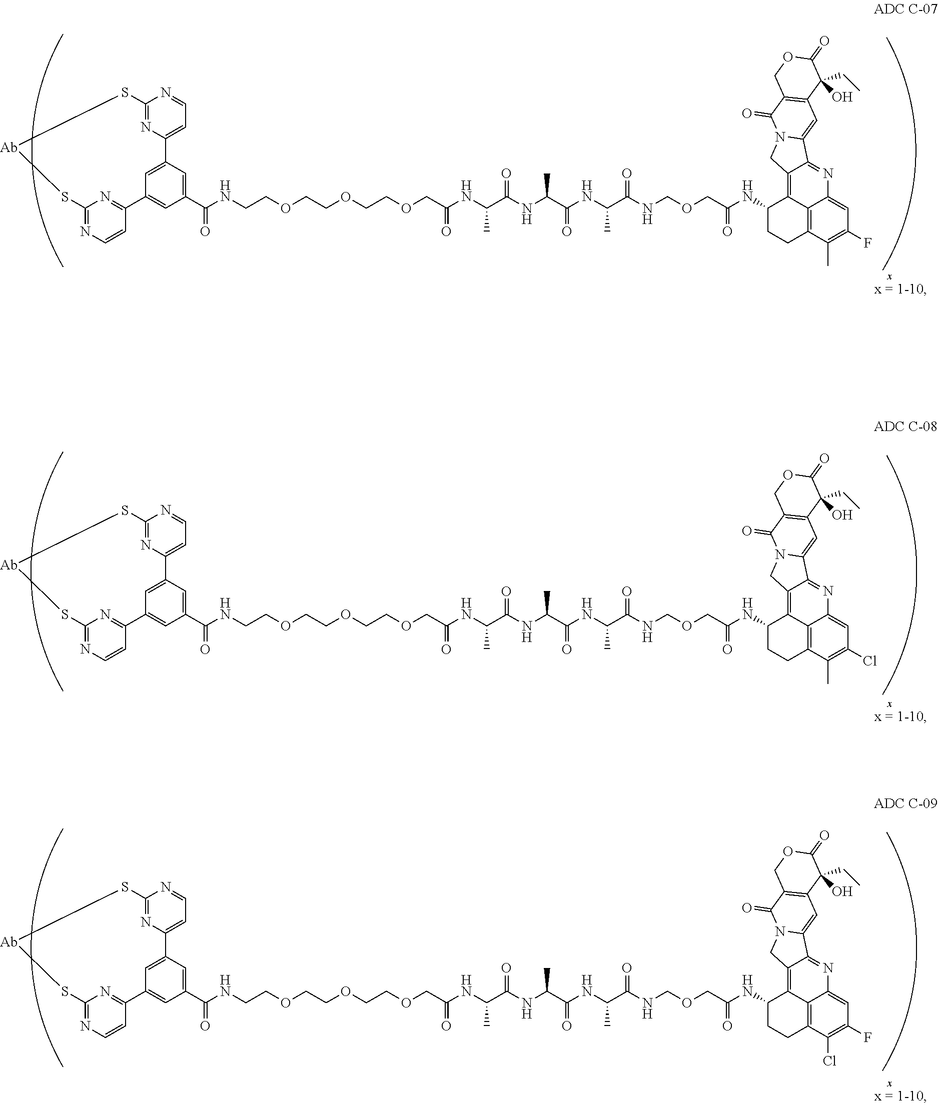

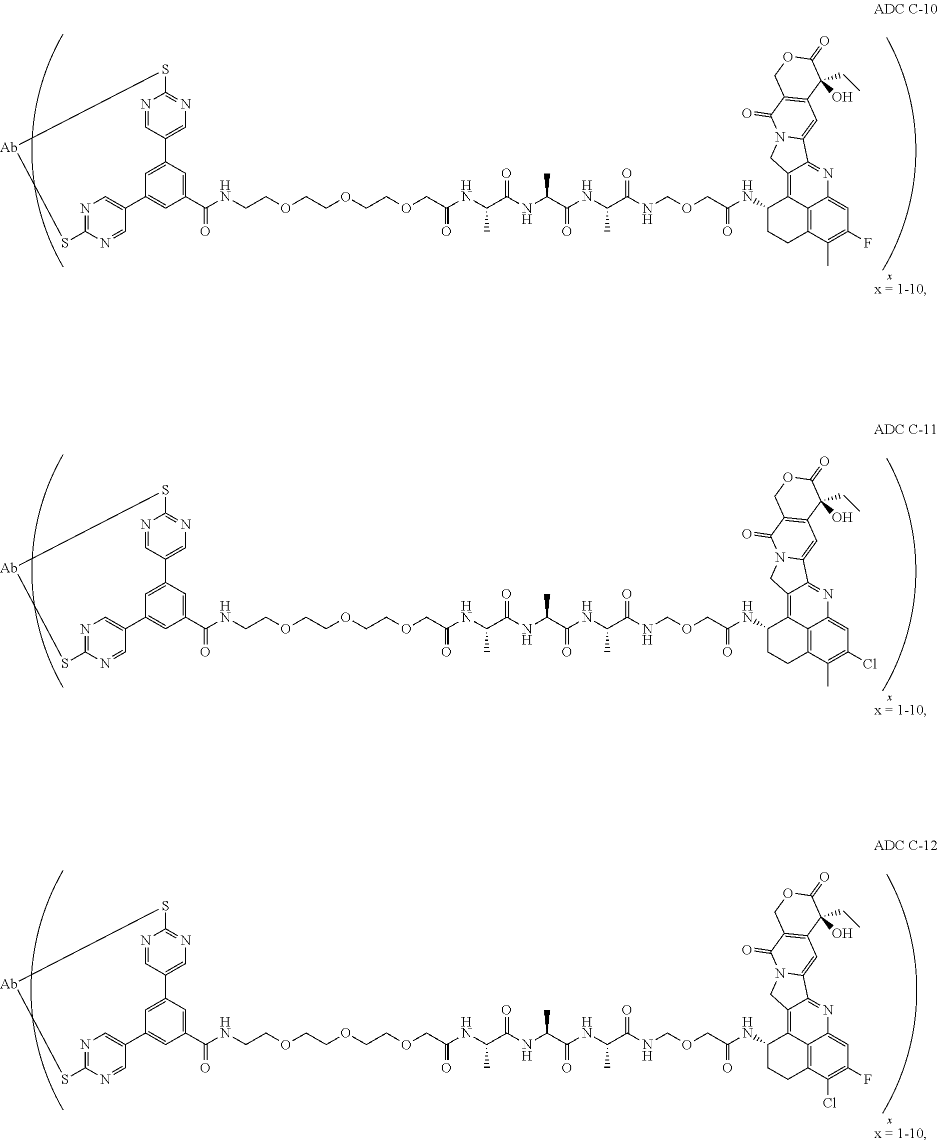

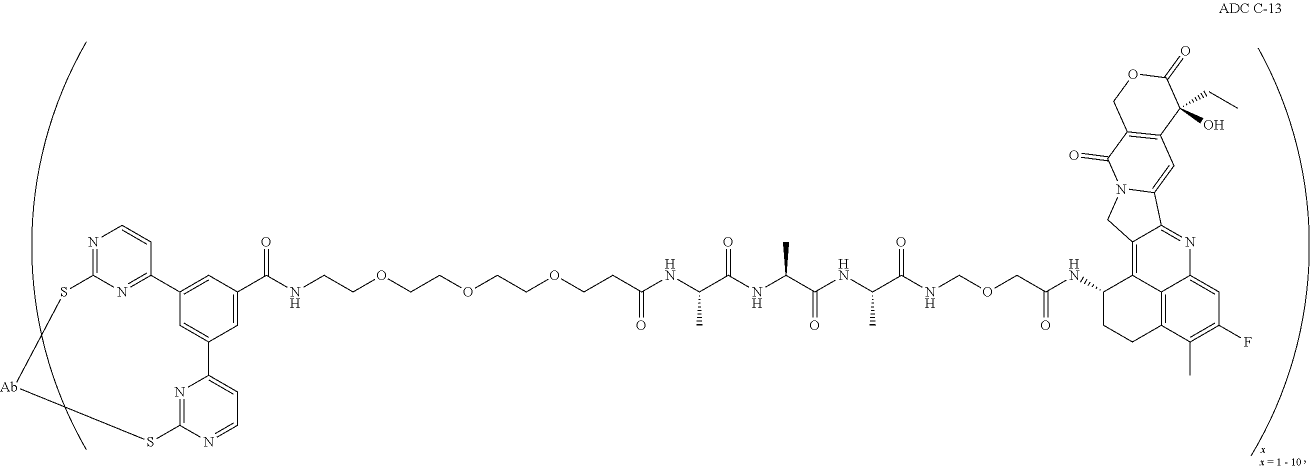

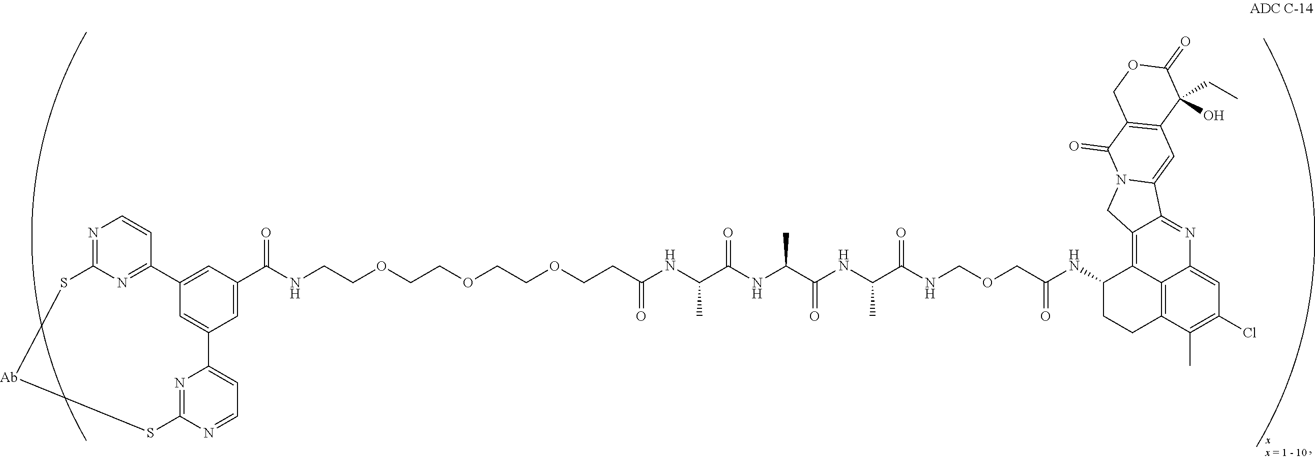

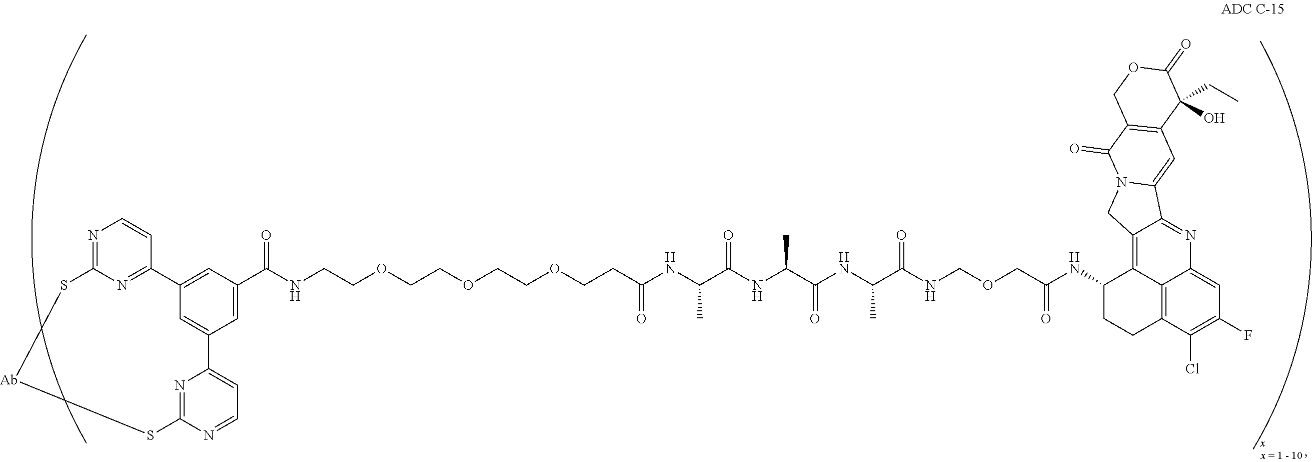

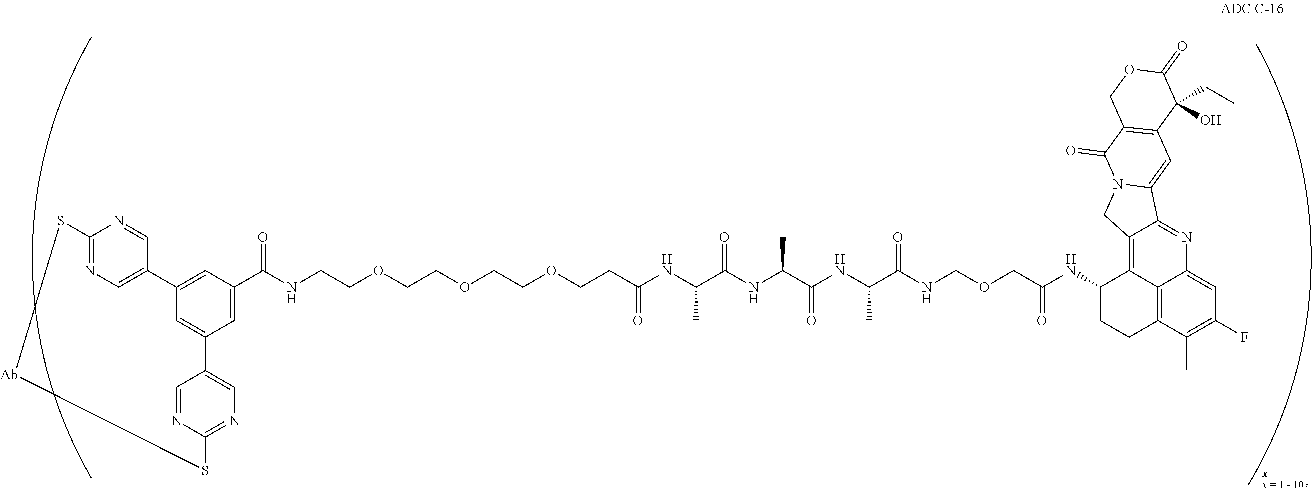

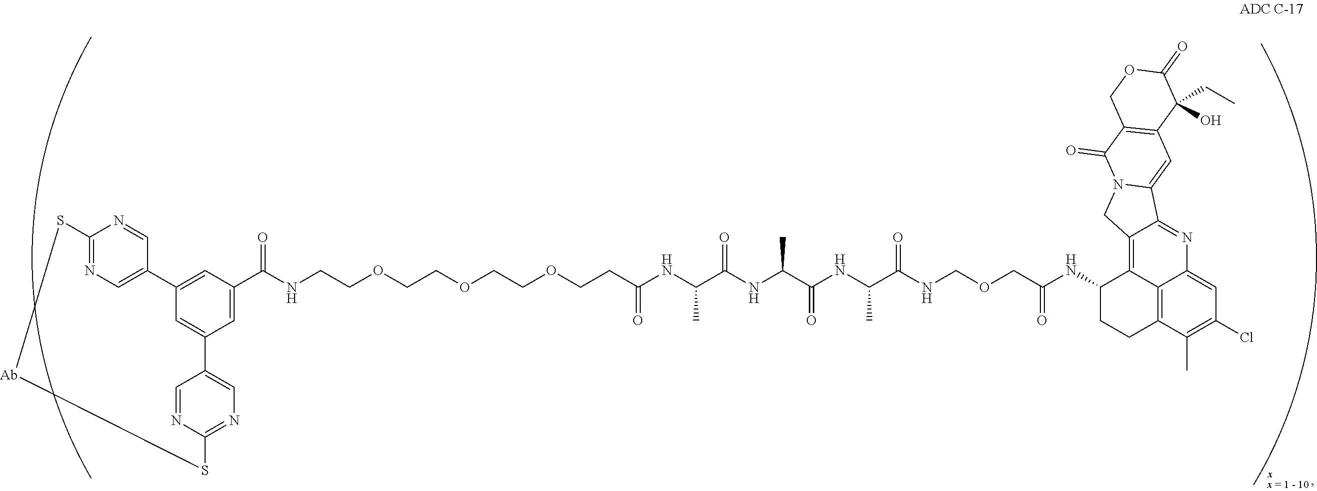

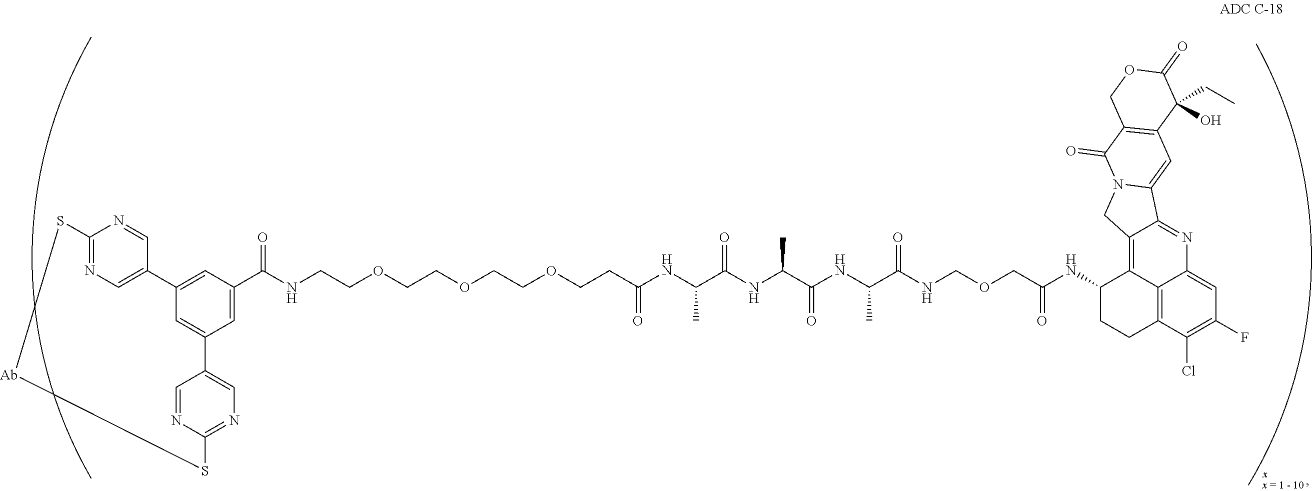

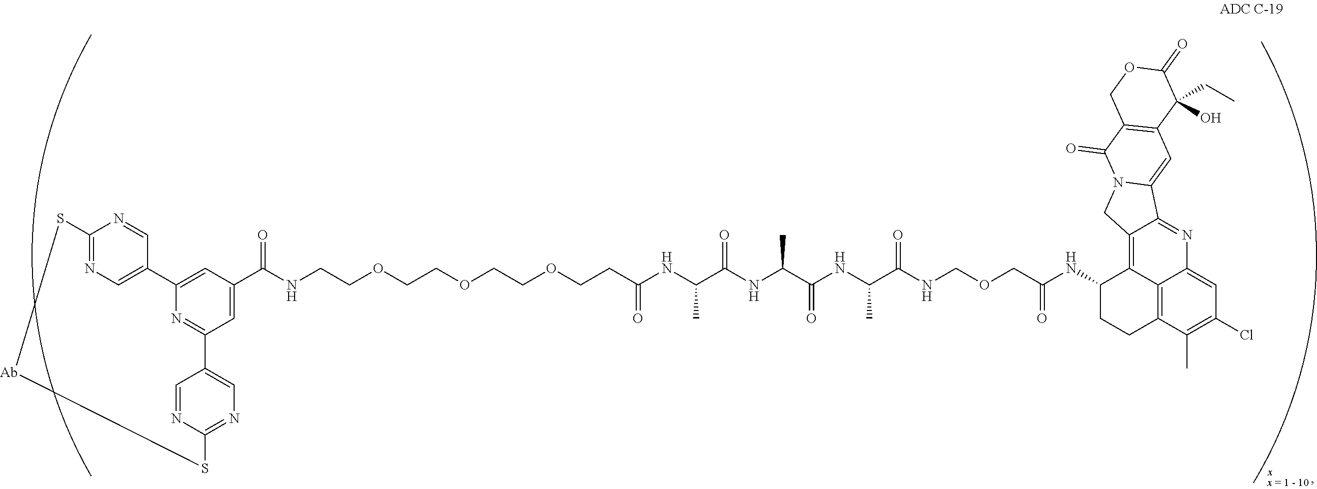

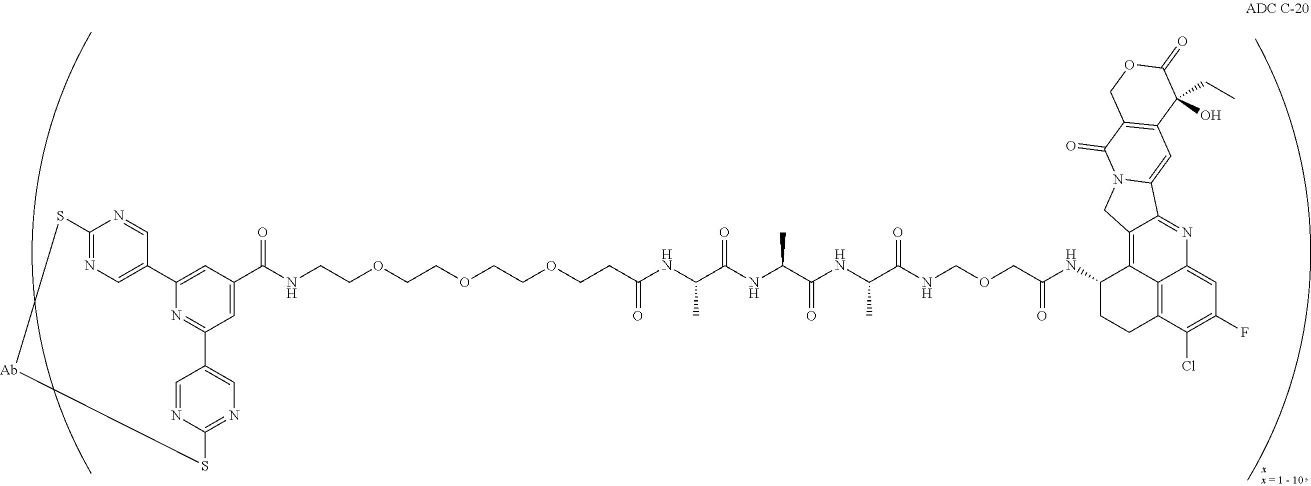

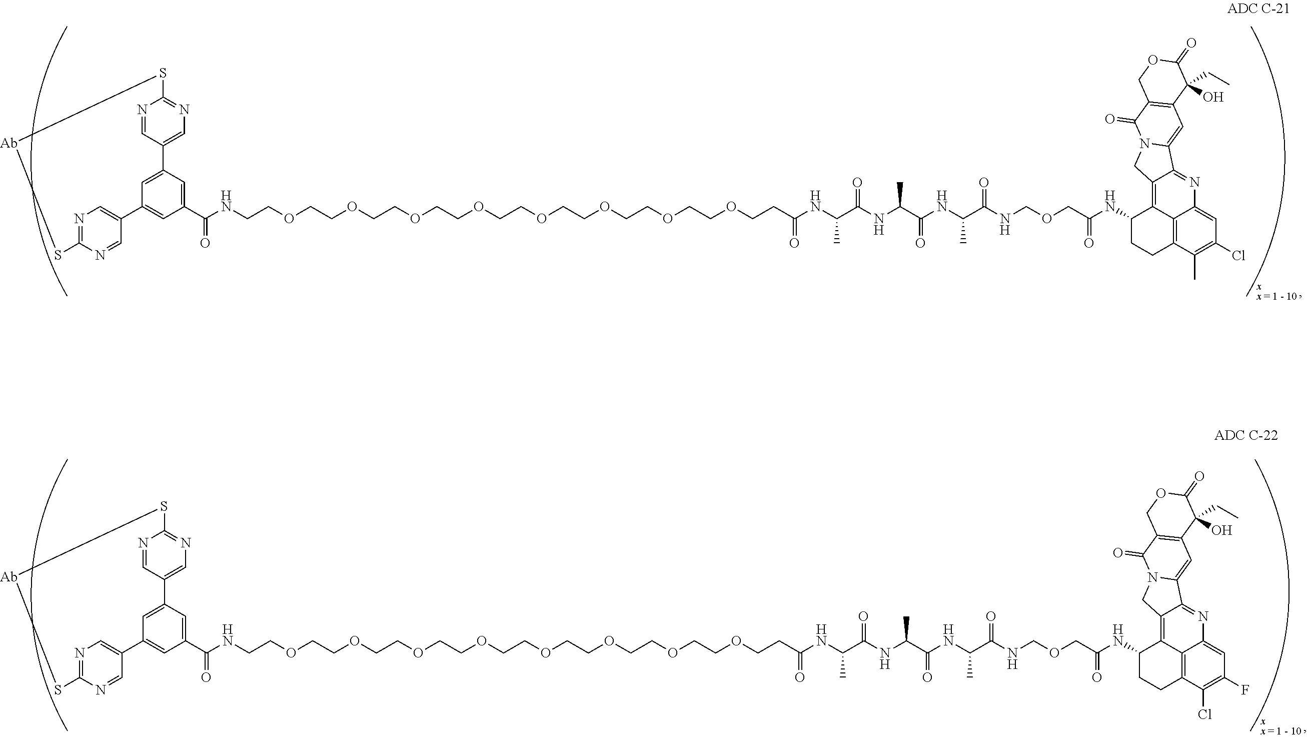

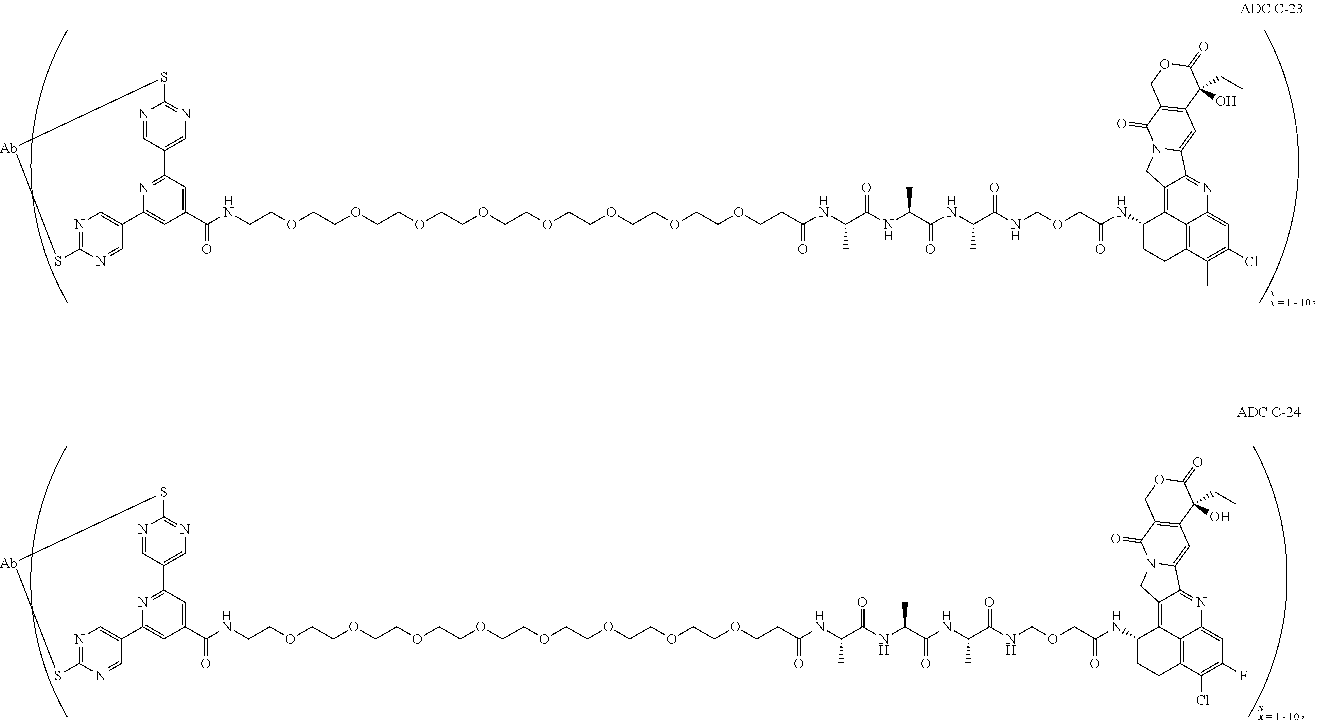

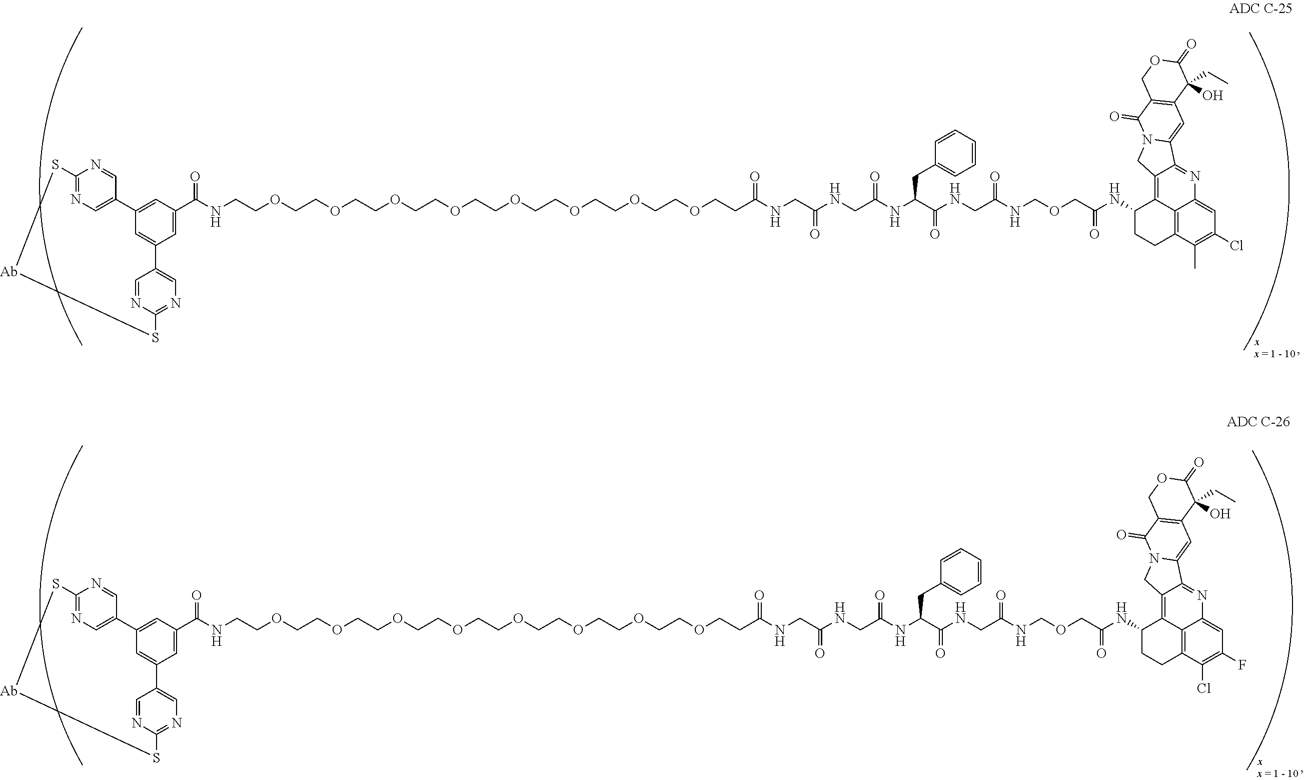

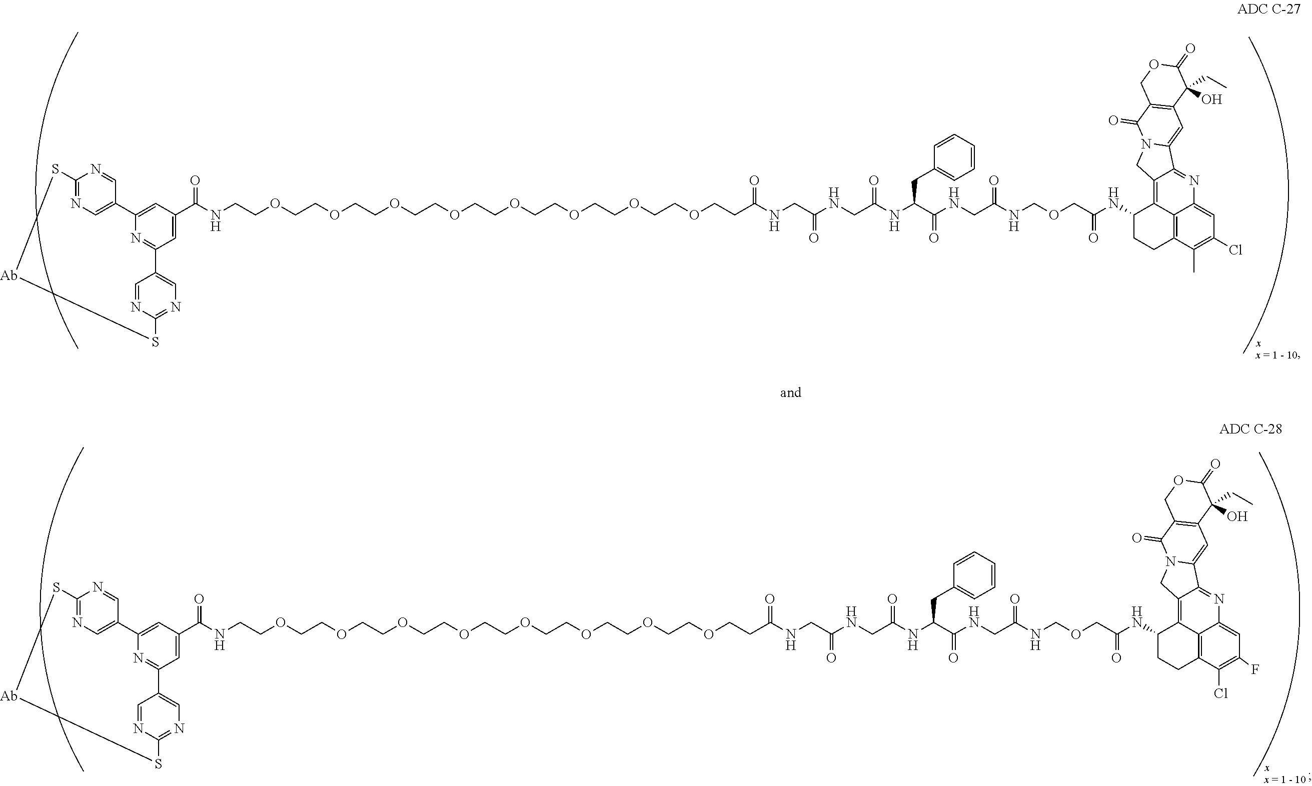

wherein, Ab is a bispecific antibody or an antigen-binding fragment thereof as defined in any one of embodiments 1-34;

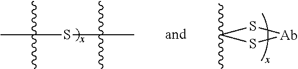

represent the specific connection mode of the sulfhydryl group of a Cys residue in the bispecific antibody or its antigen-binding fragment and M of the antibody-drug conjugate; and x represents the quantity of drug load.

Embodiment 68. The antibody-drug conjugate of embodiment 67, wherein the sulfhydryl group in the bispecific antibody or its antigen-binding fragment forms a thioether bond with M of the antibody-drug conjugate through an addition reaction or a substitution reaction to obtain the antibody-drug conjugate.

Embodiment 69. The antibody-drug conjugate of embodiment 67-68, wherein Ab is a bispecific antibody selected from the group consisting of BsAb 07B, BsAb 10B, BsAb 38B, BsAb 41B, BsAb 49B, BsAb 55B, and BsAb 56B.

Embodiment 70. An antibody-drug conjugate selected from the group consisting of ADC 07B-A-05, ADC 07B-A-14, ADC 38B-A-14, ADC 49B-A-14, ADC 49B-B-01, ADC 49B-A-05, ADC 41B-A-05, ADC 55B-A-14, and ADC 56B-A-14.

Embodiment 71. A composition comprising one or more antibody-drug conjugates according to any one of embodiments 1-70.

Embodiment 72. The composition of embodiment 71, wherein the composition has a DAR value (Drug-Antibody Conjugation Ratio) of 1 to 10.

Embodiment 73. The composition of embodiment 71, wherein the composition has a DAR value (Drug-Antibody Conjugation Ratio) of 1, 2, 3, 4, 5, 6, 7, 8, 9, 10, 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10.

Embodiment 74. The composition of embodiment 71, wherein the composition has a DAR value (Drug-Antibody Conjugation Ratio) of 3 to 9,

Embodiment 75. The composition of embodiment 71, wherein the composition has a DAR value (Drug-Antibody Conjugation Ratio) of 4 to 8,

Embodiment 76. The composition of embodiment 71, wherein the composition has a DAR value (Drug-Antibody Conjugation Ratio) of 3.0 to 3.5, 3.0 to 4.0, 3.0 to 4.5, 3.0 to 5.0, 3.0 to 5.5, 3.0 to 6.0, 3.5 to 4.0, 3.5 to 4.5, 3.5 to 5.0, 3.5 to 5.5, 3.5 to 6.0, 3.5 to 6.5, 3.5 to 7.0, 3.5 to 7.5, 3.5 to 8.0, 4.0 to 4.5, 4.0 to 5.0, 4.0 to 5.5, 4.0 to 6.0, 4.0 to 6.5, 4.0 to 7.0, 4.0 to 7.5, 4.0 to 8.0, 4.5 to 5.0, 4.5 to 5.5, 4.5 to 6.0, 4.5 to 6.5, 4.5 to 7.0, 4.5 to 7.5, 4.5 to 8.0, 5.0 to 5.5, 5.0 to 6.0, 5.0 to 6.5, 5.0 to 7.0, 5.0 to 7.5, 5.0 to 8.0, 5.5 to 6.0, 5.5 to 6.5, 5.5 to 7.0, 5.5 to 7.5, 5.5 to 8.0, 6.0 to 6.5, 6.0 to 7.0, 6.0 to 7.5, 6.0 to 8.5, 6.5 to 7.0, 6.5 to 7.5, 6.5 to 8.5, 7.0 to 7.5, 7.0 to 9.0, or 7.5 to 9.0.

Embodiment 77. A pharmaceutical composition containing one or more antibody-drug conjugates according to any one of embodiments 1-70 or the composition according to embodiment 71, and one or more pharmaceutically acceptable carriers and/or excipients.

Embodiment 78. The pharmaceutical composition of embodiment 77, wherein the pharmaceutical composition further comprises one or more additional pharmaceutically active agents selected from the group consisting of: EGFR inhibitors, HER2 inhibitors, HER3 inhibitors, HER4 inhibitors, IGFR-1 inhibitors, mTOR inhibitors, PI3 kinase inhibitors, c-MET or VEGF inhibitors, chemotherapy drugs, or any combination thereof.

Embodiment 79. A drug combination, comprising a first therapeutic agent and a second therapeutic agent, wherein the first therapeutic agent and the second therapeutic agent are administered concurrently or sequentially; the first therapeutic agent is selected from one or more of the antibody-drug conjugate of embodiments 1-70, the composition of any one of embodiments 71-76; and the second therapeutic agent is selected from the group consisting of an EGFR inhibitor, a HER2 inhibitor, a HER3 inhibitor, a HER4 inhibitor, an IGFR-1 inhibitor, an mTOR inhibitor, a PI3 kinase inhibitor, a c-MET inhibitor, a VEGF inhibitor, a chemotherapy drug, or any combination thereof.

Embodiment 80. Use of the antibody-drug conjugate according to any one of embodiments 1 to 70, the composition according to any one of embodiments 71-76, or the pharmaceutical composition according to embodiment 77 or 78, or the drug combination according to embodiment 79 in preparation of a medicament, wherein the medicament is used to prevent and/or treat and/or assist in the treatment of diseases related to c-MET and/or EGFR in a subject, and/or, for inhibiting c-MET cells in vitro or in the body of a subject or activity of c-MET and/or EGFR; wherein, the diseases related to c-MET and/or EGFR are EGFR activating mutations, EGFR gene amplification, elevated circulating HGF levels, c-MET activating mutations and/or c-MET cancers associated with MET gene amplification.

Embodiment 81. The use of according to embodiment 80, wherein the disease is selected from epithelial cell carcinoma, breast cancer, ovarian cancer, lung adenocarcinoma, small cell lung cancer, non-small cell lung cancer, oral cancer, colorectal cancer, anal cancer, prostate cancer, bladder cancer, pharyngeal cancer, nasal cancer, pancreatic cancer, skin cancer, tongue cancer, esophageal cancer, vaginal cancer, cervical cancer, spleen cancer, testicular cancer, gastric cancer, thymus cancer, thyroid cancer, hepatocellular carcinoma, sporadic or hereditary papillary renal cell carcinoma, colon cancer, liver cancer, kidney cancer, and head and neck cancer.

Embodiment 82. The use of according to embodiment 80 or 81, wherein said antibody-drug conjugate, or pharmaceutical composition and another pharmaceutical are administered in combination, either simultaneously, separately or sequentially; wherein the another pharmaceutically active agent is selected from: EGFR inhibitors, HER2 inhibitors, HER3 inhibitors, HER4 inhibitors, IGFR-1 inhibitors, mTOR inhibitors, PI3 kinase inhibitors, c-MET or VEGF inhibitors, chemotherapy drugs, or any combination thereof.

Embodiment 83. A method for inhibiting the activity of c-MET and/or EGFR in cells, comprising combining the cells with the antibody-drug conjugate of any one of embodiments 1-70, the composition according to any one of embodiments 71-76, the composition or the pharmaceutical composition of embodiment 77 or 78 or the drug combination according to embodiment 79; wherein the cells are cells that express c-MET and/or EGFR.

Embodiment 84. A method for preventing and/or treating and/or adjunctive treatment of a disease associated with c-MET and/or EGFR in a subject, the method comprising administering an effective amount to a subject in need thereof the antibody-drug conjugate according to any one of embodiments 1 to 70, the composition according to any one of embodiments 71-76, the pharmaceutical composition according to embodiment 77 or 78 or the drug combination according to embodiment 79, wherein the disease associated with c-MET and/or EGFR is a disease comprising EGFR activating mutations, EGFR gene amplification, elevated circulating HGF levels, c-MET activating mutations, and/or c-MET gene amplification cancer.

Embodiment 85. The method of embodiment 84, wherein the cancer is selected from the group consisting of epithelial cell carcinoma, breast cancer, ovarian cancer, lung adenocarcinoma, small cell lung cancer, non-small cell lung cancer, oral cancer, colorectal cancer, anal cancer, and prostate cancer, bladder cancer, pharyngeal cancer, nasal cancer, pancreatic cancer, skin cancer, tongue cancer, esophageal cancer, vaginal cancer, cervical cancer, spleen cancer, testicular cancer, gastric cancer, thymus cancer, thyroid cancer, hepatocellular carcinoma, sporadic or hereditary papillary renal cell carcinoma, colon cancer, liver cancer, kidney cancer, or head and neck cancer.

Embodiment 86. The method of embodiment 84 or 85, wherein the method further comprises administering a second therapy to the subject, the second therapy being selected from the group consisting of surgery, chemotherapy, radiotherapy, immunotherapy, gene therapy, DNA therapy, RNA therapy, nanotherapy, viral therapy, adjuvant therapy and any combination thereof, optionally, the second therapy can be applied simultaneously, separately or sequentially.

Embodiment 87. An antibody-drug conjugate according to any one of embodiments 1 to 70, the composition according to any one of embodiments 71-76, the pharmaceutical composition according to embodiment 77 or 78 or the drug combination according to embodiment 79, for preventing and/or treating and/or adjunctive treatment of a disease associated with c-MET and/or EGFR in a subject, wherein the disease associated with c-MET and/or EGFR is a disease comprising EGFR activating mutations, EGFR gene amplification, elevated circulating HGF levels, c-MET activating mutations, and/or c-MET gene amplification cancer.

Embodiment 88. The antibody-drug conjugate of embodiment 87, wherein the cancer is selected from the group consisting of epithelial cell carcinoma, breast cancer, ovarian cancer, lung adenocarcinoma, small cell lung cancer, non-small cell lung cancer, oral cancer, colorectal cancer, anal cancer, and prostate cancer, bladder cancer, pharyngeal cancer, nasal cancer, pancreatic cancer, skin cancer, tongue cancer, esophageal cancer, vaginal cancer, cervical cancer, spleen cancer, testicular cancer, gastric cancer, thymus cancer, thyroid cancer, hepatocellular carcinoma, sporadic or hereditary papillary renal cell carcinoma, colon cancer, liver cancer, kidney cancer, or head and neck cancer.

Embodiment 89. The antibody-drug conjugate of embodiment 87 or 88, wherein the method further comprises administering a second therapy to the subject, the second therapy being selected from the group consisting of surgery, chemotherapy, radiotherapy, immunotherapy, gene therapy, DNA therapy, RNA therapy, nanotherapy, viral therapy, adjuvant therapy and any combination thereof, optionally, the second therapy can be applied simultaneously, separately or sequentially.

Embodiment 90. A bispecific antibody (BsAb) conjugate comprising a bispecific antibody selected from the group consisting of. BsAb 07B, BsAb 10B, BsAb 38B, BsAb 41B, BsAb 49B, BsAb 55B, and BsAb 56B; conjugated to one or more of a linker-payload selected from the group consisting of M-01, A-05, A-07, A-14, B-01, B-02, B-03, C-10, C-17, C-19, C-21, and C-23.

Embodiment 91. A bispecific antibody (BsAb) conjugate comprising a bispecific antibody selected from the group consisting of. BsAb 07B, BsAb 10B, BsAb 38B, BsAb 41B, BsAb 49B, BsAb 55B, and BsAb 56B; conjugated to 1, 2, 3, 4, 5, 6, 7, 8, 9, or 10 molecules of a linker-payload, wherein the linker-payload is selected from the group consisting of M-01, A-05, A-07, A-14, B-01, B-02, B-03, C-10, C-17, C-19, C-21, and C-23.

Embodiment 92. A method for producing a bispecific antibody (BsAb) conjugate comprising: providing a BsAb selected from the group consisting of: BsAb 07B, BsAb 10B, BsAb 38B, BsAb 41B, BsAb 49B, BsAb 55B, and BsAb 56B and a linker-payload selected from the group consisting of: M-01, A-05, A-07, A-14, B-01, B-02, B-03, C-10, C-17, C-19, C-21, and C-23; mixing the BsAb with the linker-payload to provide a reaction mixture; incubating the reaction mixture under conditions suitable for producing the BsAb conjugate; and isolating the BsAb conjugate from the reaction mixture to produce the BsAb conjugate.

Embodiment 93. A method for producing a bispecific antibody (BsAb) conjugate having an approximate DAR of 1, 2, 3, 4, 5, 6, 7, 8, 9, or 10, comprising: providing a BsAb selected from the group consisting of: BsAb 07B, BsAb 10B, BsAb 38B, BsAb 41B, BsAb 49B, BsAb 55B, and BsAb 56B and a linker-payload selected from the group consisting of: M-01, A-05, A-07, A-14, B-01, B-02, B-03, C-10, C-17, C-19, C-21, and C-23; mixing an amount of the BsAb with an amount of the linker-payload to provide a reaction mixture capable of producing a BsAb conjugate having an approximate DAR of 1, 2, 3, 4, 5, 6, 7, 8, 9, or 10; incubating the reaction mixture under conditions suitable for producing the BsAb conjugate having the approximate DAR of 1, 2, 3, 4, 5, 6, 7, 8, 9, or 10; and isolating the BsAb conjugate from the reaction mixture to produce the BsAb conjugate having an approximate DAR of 1, 2, 3, 4, 5, 6, 7, 8, 9, or 10.

### Sequence Information

A description of the sequences covered by various embodiments of the bispecific antibodies comprising the ADCs herein is provided in the table below.

| SEQ | | |
|---|---|---|
| ID | | |
| NO: | Description | Amino acid sequence |
| 1 | LC for BsAb 07B, | DIQMTQSPSSLSASVGDRVTITCKSSQSLLYTSSQKNY |
| | 49B, and 55B | LAWYQQKPGKAPKLLIYWASTRESGVPSRFSGSGSGTD |
| | [anti-c-MET-VL: CL] | FTLTISSLQPEDFATYYCQQYYAYPWTFGQGTKVEIKR |
| | | TVAAPSVFIFPPSDEQLKSGTASVVCLLNNFYPREAKV |
| | | QWKVDNALQSGNSQESVTEQDSKDSTYSLSSTLTLSKA |
| | | DYEKHKVYACEVTHQGLSSPVTKSFNRGEC |
| 2 | HC for BsAb 07B | EVQLVESGGGLVQPGGSLRLSCAASGYTFTSYWLHWVR |
| | [anti-c-MET-VH: | QAPGKGLEWVGMIDPSNSDTRFNPNFKDRFTISADTSK |
| | Fc: D356E: L358M: | NTAYLQMNSLRAEDTAVYYCATYRSYVTPLDYWGQGTL |
| | KIH-knob: | VTVSSASTKGPSVFPLAPSSKSTSGGTAALGCLVKDYF |
| | S354C: T366W | PEPVTVSWNSGALTSGVHTFPAVLQSSGLYSLSSVVTV |
| | (Fc Eu numbering | PSSSLGTQTYICNVNHKPSNTKVDKKVEPKSCDKTHTC |
| | scheme)] | PPCPAPELLGGPSVFLFPPKPKDTLMISRTPEVTCVVV |
| | | DVSHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPCREEMTKNQVSLWCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| 3 | scFv-Fc for BsAb0 | AIQLTQSPSSLSASVGDRVTITCRASQDISSALVWYQQ |
| | 7B | KPGKAPKLLIYDASSLESGVPSRFSGSESGTDFTLTIS |
| | [anti-EGFR-VL: anti- | SLQPEDFATYYCQQFNSYPLTFGGGTKVEIKGGGGSGG |
| | EGFR-VH: | GGSGGGGSQVQLVESGGGVVQPGRSLRLSCAASGFTFS |
| | Fc: D356E: L358M: KI | TYGMHWVRQAPGKGLEWVAVIWDDGSYKYYGDSVKGRF |
| | H-hole: Y349C: | TISRDNSKNTLYLQMNSLRAEDTAVYYCARDGITMVRG |
| | T366S: L368A: Y407 | VMKDYFDYWGQGTLVTVSSGGGGDKTHTCPPCPAPELL |
| | V (Fc Eu numbering | GGPSVFLFPPKPKDTLMISRTPEVTCVVVDVSHEDPEV |
| | scheme)] | KFNWYVDGVEVHNAKTKPREEQYNSTYRVVSVLTVLHQ |
| | | DWLNGKEYKCKVSNKALPAPIEKTISKAKGQPREPQVC |
| | | TLPPSREEMTKNQVSLSCAVKGFYPSDIAVEWESNGQP |
| | | ENNYKTTPPVLDSDGSFFLVSKLTVDKSRWQQGNVFSC |
| | | SVMHEALHNHYTQKSLSLSPGK |
| 4 | LC for BsAb 10B, | AIQLTQSPSSLSASVGDRVTITCRASQDISSALVWYQQ |
| | 38B, and 41B | KPGKAPKLLIYDASSLESGVPSRFSGSESGTDFTLTIS |
| | [anti-EGFR-VL: CL] | SLQPEDFATYYCQQFNSYPLTFGGGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| 5 | HC for BsAb10B | QVQLVESGGGVVQPGRSLRLSCAASGFTFSTYGMHWVR |
| | [anti-EGFR-VH: | QAPGKGLEWVAVIWDDGSYKYYGDSVKGRFTISRDNSK |
| | Fc: D356E: L358M: | NTLYLQMNSLRAEDTAVYYCARDGITMVRGVMKDYFDY |
| | KIH-knob: | WGQGTLVTVSSASTKGPSVFPLAPSSKSTSGGTAALGC |
| | S354C: T366W | LVKDYFPEPVTVSWNSGALTSGVHTFPAVLQSSGLYSL |
| | (Fc Eu numbering | SSVVTVPSSSLGTQTYICNVNHKPSNTKVDKKVEPKSC |
| | scheme)] | DKTHTCPPCPAPELLGGPSVFLFPPKPKDTLMISRTPE |
| | | VTCVVVDVSHEDPEVKFNWYVDGVEVHNAKTKPREEQY |
| | | NSTYRVVSVLTVLHQDWLNGKEYKCKVSNKALPAPIEK |
| | | TISKAKGQPREPQVYTLPPCREEMTKNQVSLWCLVKGF |
| | | YPSDIAVEWESNGQPENNYKTTPPVLDSDGSFFLYSKL |
| | | TVDKSRWQQGNVFSCSVMHEALHNHYTQKSLSLSPGK |
| 6 | scFv-Fc for BsAb | DIQMTQSPSSLSASVGDRVTITCKSSQSLLYTSSQKNY |
| | 10B [anti-c-MET- | LAWYQQKPGKAPKLLIYWASTRESGVPSRFSGSGSGTD |
| | VL: anti-c-MET-VH: | FTLTISSLQPEDFATYYCQQYYAYPWTFGQGTKVEIKG |
| | Fc: D356E: L358M: | GGGSGGGGSGGGGSEVQLVESGGGLVQPGGSLRLSCAA |
| | KIH-hole: Y349C: | SGYTFTSYWLHWVRQAPGKGLEWVGMIDPSNSDTRFNP |
| | T366S: L368A: Y407 | NFKDRFTISADTSKNTAYLQMNSLRAEDTAVYYCATYR |
| | V (Fc Eu numbering | SYVTPLDYWGQGTLVTVSSGGGGDKTHTCPPCPAPELL |
| | scheme)] | GGPSVFLFPPKPKDTLMISRTPEVTCVVVDVSHEDPEV |
| | | KFNWYVDGVEVHNAKTKPREEQYNSTYRVVSVLTVLHQ |
| | | DWLNGKEYKCKVSNKALPAPIEKTISKAKGQPREPQVC |
| | | TLPPSREEMTKNQVSLSCAVKGFYPSDIAVEWESNGQP |
| | | ENNYKTTPPVLDSDGSFFLVSKLTVDKSRWQQGNVFSC |
| | | SVMHEALHNHYTQKSLSLSPGK |
| 7 | HC for BsAb 38B | QVQLVESGGGVVQPGRSLRLSCAASGFTFSTYGMHWVR |
| | and 41B [anti-EGFR- | QAPGKGLEWVAVIWDDGSYKYYGDSVKGRFTISRDNSK |
| | VH: | NTLYLQMNSLRAEDTAVYYCARDGITMVRGVMKDYFDY |
| | Fc: L234A: L235A: P3 | WGQGTLVTVSSASTKGPSVFPLAPSSKSTSGGTAALGC |
| | 29A: KIH- | LVKDYFPEPVTVSWNSGALTSGVHTFPAVLQSSGLYSL |
| | knob: S354C: T366W | SSVVTVPSSSLGTQTYICNVNHKPSNTKVDKKVEPKSC |
| | (Fc Eu numbering | DKTHTCPPCPAPEAAGGPSVFLFPPKPKDTLMISRTPE |
| | scheme)] | VTCVVVDVSHEDPEVKFNWYVDGVEVHNAKTKPREEQY |
| | | NSTYRVVSVLTVLHQDWLNGKEYKCKVSNKALAAPIEK |
| | | TISKAKGQPREPQVYTLPPCRDELTKNQVSLWCLVKGF |
| | | YPSDIAVEWESNGQPENNYKTTPPVLDSDGSFFLYSKL |
| | | TVDKSRWQQGNVFSCSVMHEALHNHYTQKSLSLSPGK |
| 8 | scFv-Fc for BsAb | DIQMTQSPSSLSASVGDRVTITCKSSQSLLYTSSQKNY |
| | 38B and 56B | LAWYQQKPGKAPKLLIYWASTRESGVPSRFSGSGSGTD |
| | [anti-c-MET-VL: anti- | FTLTISSLQPEDFATYYCQQYYAYPWTFGQGTKVEIKG |
| | c-MET-VH: | GGGSGGGGSGGGGSEVQLVESGGGLVQPGGSLRLSCAA |
| | Fc: L234A: L235A: P3 | SGYTFTSYWLHWVRQAPGKGLEWVGMIDPSNSDTRFNP |
| | 29A: KIH- | NFKDRFTISADTSKNTAYLQMNSLRAEDTAVYYCATYR |
| | hole: Y349C: | SYVTPLDYWGQGTLVTVSSGGGGDKTHTCPPCPAPEAA |
| | T366S: L368A: Y407 | GGPSVFLFPPKPKDTLMISRTPEVTCVVVDVSHEDPEV |
| | V (Fc Eu numbering | KFNWYVDGVEVHNAKTKPREEQYNSTYRVVSVLTVLHQ |
| | scheme)] | DWLNGKEYKCKVSNKALAAPIEKTISKAKGQPREPQVC |
| | | TLPPSRDELTKNQVSLSCAVKGFYPSDIAVEWESNGQP |
| | | ENNYKTTPPVLDSDGSFFLVSKLTVDKSRWQQGNVFSC |
| | | SVMHEALHNHYTQKSLSLSPGK |
| 9 | HC for BsAb 49B | EVQLVESGGGLVQPGGSLRLSCAASGYTFTSYWLHWVR |
| | and 55B [anti-c- | QAPGKGLEWVGMIDPSNSDTRFNPNFKDRFTISADTSK |
| | MET-VH: | NTAYLQMNSLRAEDTAVYYCATYRSYVTPLDYWGQGTL |
| | Fc: L234A: L235A: P3 | VTVSSASTKGPSVFPLAPSSKSTSGGTAALGCLVKDYF |
| | 29A: KIH- | PEPVTVSWNSGALTSGVHTFPAVLQSSGLYSLSSVVTV |
| | knob: S354C: T366W | PSSSLGTQTYICNVNHKPSNTKVDKKVEPKSCDKTHTC |
| | (Fc Eu numbering | PPCPAPEAAGGPSVFLFPPKPKDTLMISRTPEVTCVVV |
| | scheme)] | DVSHEDPEVKFNWYVDGVEVHNAKTKPREEQYNSTYRV |
| | | VSVLTVLHQDWLNGKEYKCKVSNKALAAPIEKTISKAK |
| | | GQPREPQVYTLPPCRDELTKNQVSLWCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| 10 | scFv-Fc for BsAb | AIQLTQSPSSLSASVGDRVTITCRASQDISSALVWYQQ |
| | 49B | KPGKAPKLLIYDASSLESGVPSRFSGSESGTDFTLTIS |
| | [anti-EGFR-VL: anti- | SLQPEDFATYYCQQFNSYPLTFGGGTKVEIKGGGGSGG |
| | EGFR-VH: | GGSGGGGSQVQLVESGGGVVQPGRSLRLSCAASGFTFS |
| | Fc: L234A: L235A: P3 | TYGMHWVRQAPGKGLEWVAVIWDDGSYKYYGDSVKGRF |
| | 29A: KIH- | TISRDNSKNTLYLQMNSLRAEDTAVYYCARDGITMVRG |
| | hole: Y349C: | VMKDYFDYWGQGTLVTVSSGGGGDKTHTCPPCPAPEAA |
| | T366S: L368A: Y407 | GGPSVFLFPPKPKDTLMISRTPEVTCVVVDVSHEDPEV |
| | V (Fc Eu numbering | KFNWYVDGVEVHNAKTKPREEQYNSTYRVVSVLTVLHQ |
| | scheme)] | DWLNGKEYKCKVSNKALAAPIEKTISKAKGQPREPQVC |
| | | TLPPSRDELTKNQVSLSCAVKGFYPSDIAVEWESNGQP |
| | | ENNYKTTPPVLDSDGSFFLVSKLTVDKSRWQQGNVFSC |
| | | SVMHEALHNHYTQKSLSLSPGK |
| 11 | scFab-Fc for BsAb | DIQMTQSPSSLSASVGDRVTITCKSSQSLLYTSSQKNY |
| | 41B | LAWYQQKPGKAPKLLIYWASTRESGVPSRFSGSGSGTD |
| | [anti-cMET-VL | FTLTISSLQPEDFATYYCQQYYAYPWTFGCGTKVEIKR |
| | (Q106C): anti-c-MET- | TVAAPSVFIFPPSDEQLKSGTASVVCLLNNFYPREAKV |
| | VH (G304C): | QWKVDNALQSGNSQESVTEQDSKDSTYSLSSTLTLSKA |
| | Fc: L234A: L235A: P3 | DYEKHKVYACEVTHQGLSSPVTKSFNRGECGGGGSGGG |
| | 29A: KIH- | GSGGGGSGGGGSGGGGSGGGGSGGGGSGGGGSEVQLVE |
| | hole: Y349C: | SGGGLVQPGGSLRLSCAASGYTFTSYWLHWVRQAPGKC |
| | T366S: L368A: Y407V | LEWVGMIDPSNSDTRFNPNFKDRFTISADTSKNTAYLQ |
| | (Fc Eu numbering | MNSLRAEDTAVYYCATYRSYVTPLDYWGQGTLVTVSSA |
| | scheme)] | STKGPSVFPLAPSSKSTSGGTAALGCLVKDYFPEPVTV |
| | | SWNSGALTSGVHTFPAVLQSSGLYSLSSVVTVPSSSLG |
| | | TQTYICNVNHKPSNTKVDKKVEPKSCDKTHTCPPCPAP |
| | | EAAGGPSVFLFPPKPKDTLMISRTPEVTCVVVDVSHED |
| | | PEVKFNWYVDGVEVHNAKTKPREEQYNSTYRVVSVLTV |
| | | LHQDWLNGKEYKCKVSNKALAAPIEKTISKAKGQPREP |
| | | QVCTLPPSRDELTKNQVSLSCAVKGFYPSDIAVEWESN |
| | | GQPENNYKTTPPVLDSDGSFFLVSKLTVDKSRWQQGNV |
| | | FSCSVMHEALHNHYTQKSLSLSPGK |
| 12 | VL-CH1 for BsAb | AIQLTQSPSSLSASVGDRVTITCRASQDISSALVWYQQ |
| | 55B [anti-EGFR- | KPGKAPKLLIYDASSLESGVPSRFSGSESGTDFTLTIS |
| | VL: CH1] | SLQPEDFATYYCQQFNSYPLTFGGGTKVEIKSSASTKG |
| | | PSVFPLAPSSKSTSGGTAALGCLVKDYFPEPVTVSWNS |
| | | GALTSGVHTFPAVLQSSGLYSLSSVVTVPSSSLGTQTY |
| | | ICNVNHKPSNTKVDKKVEPKSC |
| 13 | VH-CL-Fc for BsAb | QVQLVESGGGVVQPGRSLRLSCAASGFTFSTYGMHWVR |
| | 55B [anti-EGFR- | QAPGKGLEWVAVIWDDGSYKYYGDSVKGRFTISRDNSK |
| | VH: CL: | NTLYLQMNSLRAEDTAVYYCARDGITMVRGVMKDYFDY |
| | Fc: L234A: L235A: | WGQGTLVTVSSASVAAPSVFIFPPSDEQLKSGTASVVC |
| | P329A: KIH- | LLNNFYPREAKVQWKVDNALQSGNSQESVTEQDSKDST |
| | hole: Y349C: | YSLSSTLTLSKADYEKHKVYACEVTHQGLSSPVTKSFN |
| | T366S: L368A: Y407V | RGECDKTHTCPPCPAPEAAGGPSVFLFPPKPKDTLMIS |
| | (Fc Eu numbering | RTPEVTCVVVDVSHEDPEVKFNWYVDGVEVHNAKTKPR |
| | scheme)] | EEQYNSTYRVVSVLTVLHQDWLNGKEYKCKVSNKALAA |
| | | PIEKTISKAKGQPREPQVCTLPPSRDELTKNQVSLSCA |
| | | VKGFYPSDIAVEWESNGQPENNYKTTPPVLDSDGSFFL |
| | | VSKLTVDKSRWQQGNVFSCSVMHEALHNHYTQKSLSLS |
| | | PGK |
| 14 | scFv-Fc for BsAb | AIQLTQSPSSLSASVGDRVTITCRASQDISSALVWYQQ |
| | 56B [anti-EGFR- | KPGKAPKLLIYDASSLESGVPSRFSGSESGTDFTLTIS |
| | VL: anti-EGFR-VH: | SLQPEDFATYYCQQFNSYPLTFGGGTKVEIKGGGGSGG |
| | Fc: L234A: L235A: P3 | GGSGGGGSQVQLVESGGGVVQPGRSLRLSCAASGFTFS |
| | 29A: KIH- | TYGMHWVRQAPGKGLEWVAVIWDDGSYKYYGDSVKGRF |
| | knob: S354C: T366W | TISRDNSKNTLYLQMNSLRAEDTAVYYCARDGITMVRG |
| | (Fc Eu numbering | VMKDYFDYWGQGTLVTVSSGGGGDKTHTCPPCPAPEAA |
| | scheme)] | GGPSVFLFPPKPKDTLMISRTPEVTCVVVDVSHEDPEV |
| | | KFNWYVDGVEVHNAKTKPREEQYNSTYRVVSVLTVLHQ |
| | | DWLNGKEYKCKVSNKALAAPIEKTISKAKGQPREPQVY |
| | | TLPPCRDELTKNQVSLWCLVKGFYPSDIAVEWESNGQP |
| | | ENNYKTTPPVLDSDGSFFLYSKLTVDKSRWQQGNVFSC |
| | | SVMHEALHNHYTQKSLSLSPGK |
| 15 | Anti-EGFR-VL | AIQLTQSPSSLSASVGDRVTITCRASQDISSALVWYQQ |
| | | KPGKAPKLLIYDASSLESGVPSRFSGSESGTDFTLTIS |
| | | SLQPEDFATYYCQQFNSYPLTFGGGTKVEIK |
| 16 | Anti-EGFR-VH | QVQLVESGGGVVQPGRSLRLSCAASGFTFSTYGMHWVR |
| | | QAPGKGLEWVAVIWDDGSYKYYGDSVKGRFTISRDNSK |
| | | NTLYLQMNSLRAEDTAVYYCARDGITMVRGVMKDYFDY |
| | | WGQGTLVTVSS |
| 17 | Anti-c-MET-VL | DIQMTQSPSSLSASVGDRVTITCKSSQSLLYTSSQKNY |
| | | LAWYQQKPGKAPKLLIYWASTRESGVPSRFSGSGSGTD |
| | | FTLTISSLQPEDFATYYCQQYYAYPWTFGQGTKVEIK |
| 18 | Anti-c-MET-VH | EVQLVESGGGLVQPGGSLRLSCAASGYTFTSYWLHWVR |
| | | QAPGKGLEWVGMIDPSNSDTRFNPNFKDRFTISADTSK |
| | | NTAYLQMNSLRAEDTAVYYCATYRSYVTPLDYWGQGTL |
| | | VTVSS |
| 19 | Anti-EGFR-CDR-L1 | RASQDISSALV |
| | (Kabat/Chothia/Abm | |
| | definition) | |
| 20 | Anti-EGFR-CDR-L1 | QDISSA |
| | (IMGT definition) | |
| 21 | Anti-EGFR-CDR-L2 | DASSLES |
| | (Kabat/Chothia/Abm | |
| | definition) | |
| 22 | Anti-EGFR-CDR-L2 | DAS |
| | (IMGT definition) | |
| 23 | Anti-EGFR-CDR-L3 | QQFNSYPLT |
| | (Kabat/Chothia/Abm/ | |
| | IMGT definition) | |
| 24 | Anti-EGFR-CDR-H1 | TYGMH |
| | (Kabat definition) | |
| 25 | Anti-EGFR-CDR-H1 | GFTFSTY |
| | (Chothia definition) | |
| 26 | Anti-EGFR-CDR-H1 | GFTFSTYG |
| | (IMGT definition) | |
| 27 | Anti-EGFR-CDR-H1 | GFTFSTYGMH |
| | (Abm definition) | |
| 28 | Anti-EGFR-CDR-H2 | VIWDDGSYKYYGDSVKG |
| | (Kabat definition) | |
| 29 | Anti-EGFR-CDR-H2 | WDDGSY |
| | (Chothia definition) | |
| 30 | Anti-EGFR-CDR-H2 | IWDDGSYK |
| | (IMGT definition) | |
| 31 | Anti-EGFR-CDR-H2 | VIWDDGSYKY |
| | (Abm definition) | |
| 32 | Anti-EGFR-CDR-H3 | DGITMVRGVMKDYFDY |
| | (Kabat/Chothia/Abm | |
| | definition) | |
| 33 | Anti-EGFR-CDR-H3 | ARDGITMVRGVMKDYFDY |
| | (IMGT definition) | |
| 34 | Anti-c-MET-CDR-L1 | KSSQSLLYTSSQKNYLA |
| | (Kabat/Chothia/Abm | |
| | definition) | |
| 35 | Anti-c-MET-CDR-L1 | QSLLYTSSQKNY |
| | (IMGT definition) | |
| 36 | Anti-c-MET-CDR-L2 | WASTRES |
| | (Kabat/Chothia/Abm | |
| | definition) | |
| 37 | Anti-c-MET-CDR-L2 | WAS |
| | (IMGT definition) | |
| 38 | Anti-c-MET-CDR-L3 | QQYYAYPWT |
| | (Kabat/Chothia/Abm/ | |
| | IMGT definition) | |
| 39 | Anti-c-MET-CDR- | SYWLH |
| | H1 (Kabat definition) | |
| 40 | Anti-c-MET-CDR-H1 | GYTFTSY |
| | (Chothia definition) | |
| 41 | Anti-c-MET-CDR-H1 | GYTFTSYW |
| | (IMGT definition) | |
| 42 | Anti-c-MET-CDR- | GYTFTSYWLH |
| | H1 (Abm definition) | |
| 43 | Anti-c-MET-CDR- | MIDPSNSDTRFNPNFKD |
| | H2 (Kabat definition) | |
| 44 | Anti-c-MET-CDR-H2 | DPSNSD |
| | (Chothia definition) | |
| 45 | Anti-c-MET-CDR-H2 | IDPSNSDT |
| | (IMGT definition) | |
| 46 | Anti-c-MET-CDR- | MIDPSNSDTR |
| | H2 (Abm definition) | |
| 47 | Anti-c-MET-CDR-H3 | YRSYVTPLDY |
| | (Kabat/Chothia/Abm | |
| | definition) | |
| 48 | Anti-c-MET-CDR-H3 | ATYRSYVTPLDY |
| | (IMGT definition) | |
| 49 | Fc (DELM: KIH) | APELLGGPSVFLFPPKPKDTLMISRTPEVTCVVVDVSH |
| | (knob) for BsAb 07B | EDPEVKFNWYVDGVEVHNAKTKPREEQYNSTYRVVSVL |
| | and 10B | TVLHQDWLNGKEYKCKVSNKALPAPIEKTISKAKGQPR |
| | [Fc: D356E: L358M: K | EPQVYTLPPCREEMTKNQVSLWCLVKGFYPSDIAVEWE |
| | IH-knob: | SNGQPENNYKTTPPVLDSDGSFFLYSKLTVDKSRWQQG |
| | S354C: T366W | NVFSCSVMHEALHNHYTQKSLSLSPGK |
| | (Fc Eu numbering | |
| | scheme)] | |
| 50 | Fc (DELM: KIH) | APELLGGPSVFLFPPKPKDTLMISRTPEVTCVVVDVSH |
| | (hole) for BsAb 07B | EDPEVKFNWYVDGVEVHNAKTKPREEQYNSTYRVVSVL |
| | and 10B | TVLHQDWLNGKEYKCKVSNKALPAPIEKTISKAKGQPR |
| | [Fc: D356E: L358M: K | EPQVCTLPPSREEMTKNQVSLSCAVKGFYPSDIAVEWE |
| | IH-hole (Y349C: | SNGQPENNYKTTPPVLDSDGSFFLVSKLTVDKSRWQQG |
| | T366S: L368A: Y407 | NVFSCSVMHEALHNHYTQKSLSLSPGK |
| | V Fc Eu numbering | |
| | scheme)] | |
| 51 | Fc (LALAPA: KIH | APEAAGGPSVFLFPPKPKDTLMISRTPEVTCVVVDVSH |
| | (knob) for BsAb 38B, | EDPEVKFNWYVDGVEVHNAKTKPREEQYNSTYRVVSVL |
| | 41B, 49B, 55B and | TVLHQDWLNGKEYKCKVSNKALAAPIEKTISKAKGQPR |
| | 56B | EPQVYTLPPCRDELTKNQVSLWCLVKGFYPSDIAVEWE |
| | [Fc: L234A: L235A: P3 | SNGQPENNYKTTPPVLDSDGSFFLYSKLTVDKSRWQQG |
| | 29A: KIH- | NVFSCSVMHEALHNHYTQKSLSLSPGK |
| | knob: S354C: T366 | |
| | W | |
| | (Fc Eu numbering | |
| | scheme)] | |
| 52 | Fc (LALAPA: KIH | APEAAGGPSVFLFPPKPKDTLMISRTPEVTCVVVDVSH |
| | (hole) for BsAb 38B, | EDPEVKFNWYVDGVEVHNAKTKPREEQYNSTYRVVSVL |
| | 41B, 49B, 55B and | TVLHQDWLNGKEYKCKVSNKALAAPIEKTISKAKGQPR |
| | 56B | EPQVCTLPPSRDELTKNQVSLSCAVKGFYPSDIAVEWE |
| | [Fc: (L234A: L235A: P | SNGQPENNYKTTPPVLDSDGSFFLVSKLTVDKSRWQQG |
| | 329A): KIH-hole | NVFSCSVMHEALHNHYTQKSLSLSPGK |
| | (Y349C: | |
| | T366S: L368A: Y407 | |
| | V Fc Eu numbering | |
| | scheme)] | |
| 53 | CL for BsAb 07B, | RTVAAPSVFIFPPSDEQLKSGTASVVCLLNNFYPREAK |
| | 10B, 38B, 41B, 49B | VQWKVDNALQSGNSQESVTEQDSKDSTYSLSSTLTLS |
| | and 55B | KADYEKHKVYACEVTHQGLSSPVTKSFNRGEC |
| 54 | CH1 | ASTKGPSVFPLAPSSKSTSGGTAALGCLVKDYFPEPVT |
| | | VSWNSGALTSGVHTFPAVLQSSGLYSLSSVVTVPSSSL |
| | | GTQTYICNVNHKPSNTKVDKKVEPKSC |
| 55 | Peptide Linker-1 | GGGGSGGGGSGGGGS |
| | (GGGGS)₃ | |
| 56 | Peptide Linker-2 | GGGGSGGGGSGGGGSGGGGSGGGGSGGGGSGGGGSG |
| | (GGGGS)₈ | GGGS |
| 57 | Peptide Linker | GGGGS |
| 58 | Peptide linker | GGGGSGGGGSGGGGSGGGGS |
| | (GGGGS)₄ | |
| 59 | Anti-c-MET-VL | DIQMTQSPSSLSASVGDRVTITCKSSQSLLYTSSQKNY |
| | (Q106C) | LAWYQQKPGKAPKLLIYWASTRESGVPSRFSGSGSGTD |
| | | FTLTISSLQPEDFATYYCQQYYAYPWTFGCGTKVEIK |
| 60 | Anti-c-MET-VH | EVQLVESGGGLVQPGGSLRLSCAASGYTFTSYWLHWVR |
| | (G44C) | QAPGKCLEWVGMIDPSNSDTRFNPNFKDRFTISADTSK |
| | | NTAYLQMNSLRAEDTAVYYCATYRSYVTPLDYWGQGTL |
| | | VTVSS |
| 61 | Peptide linker | GGGG |
| 62 | Heavy chain CH1 | DKTHTCPPCP |
| | hinge region | |
| 63 | CL for BsAb 55B | ASVAAPSVFIFPPSDEQLKSGTASVVCLLNNFYPREAK |
| | | VQWKVDNALQSGNSQESVTEQDSKDSTYSLSSTLTLSK |
| | | ADYEKHKVYACEVTHQGLSSPVTKSFNRGEC |
| 64 | Mutated human IgG1 | ASTKGPSVFPLAPSSKSTSGGTAALGCLVKDYFPEPVT |
| | | VSWNSGALTSGVHTFPAVLQSSGLYSLSSVVTVPSSSL |
| | | GTQTYICNVNHKPSNTKVDKKVEPKSCDKTHTCPPCPA |
| | | PEAAGAPSVFLFPPKPKDTLMISRTPEVTCVVVDVSHE |
| | | DPEVKFNWYVDGVEVHNAKTKPREEQYNSTYRVVSVLT |
| | | VLHQDWLNGKEYKCKVSNKALPAPIEKTISKAKGQPRE |
| | | PQVYTLPPSRDELTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| | heavy chain constant | |
| | region | |
| 65 | Peptide linker | GGGGSGGGGS |
| | (GGGGS)₂ | |
| 66 | Peptide linker | GGGGSGGGGSGGGGSGGGGSGGGGS |
| | (GGGGS)₅ | |
| 67 | Peptide linker | GGGGSGGGGSGGGGSGGGGSGGGGSGGGGS |
| | (GGGGS)₆ | |
| 68 | Peptide linker | GGGGSGGGGSGGGGSGGGGSGGGGSGGGGSGGGGS |
| | (GGGGS)₇ | |
| 69 | Peptide linker | GGGGSGGGGSGGGGSGGGGSGGGGSGGGGSGGGGSG |
| | (GGGGS)₉ | GGGSGGGGS |
| 70 | Peptide linker | GGGGSGGGGSGGGGSGGGGSGGGGSGGGGSGGGGSG |
| | (GGGGS)₁₀ | GGGSGGGGSGGGGS |
| 71 | Gly-Gly-Phe-Gly | GGFG |
| | peptide 1 | |
| 72 | Gly-Gly-Val-Ala | GGVA |
| | peptide 2 | |
| 73 | Gly-Phe-Leu-Gly | GFLG |
| | peptide 3 | |
| 74 | Glu-Ala-Ala-Ala | EAAA |
| | peptide 4 | |
| 75 | Gly-Gly-Gly-Gly-Gly | GGGGG |
| | peptide 5 | |

### EXAMPLES

The present invention will be further described below through the description of specific embodiments, but this is not intended to limit the invention. Those skilled in the art can make various modifications or improvements based on the teachings of the present invention without departing from the basic idea and scope of the present invention.

The structures of the compounds described in the following examples were determined by NMR (¹H NMR) or mass spectrometry (MS).

Nuclear Magnetic Resonance (¹H NMR) was measured using a Bruker 400 MHz nuclear magnetic resonance instrument; the deuterated reagent was hexadeuterated dimethyl sulfoxide (DMSO-d6); the internal standard substance was tetramethylsilane (TMS).

The abbreviations in the nuclear magnetic resonance (NM/R) spectra used in the examples are shown below.
s: Single peak (singlet), d: Doublet, t: Triplet, q: Quartet, m: Multiplet, br: Broad, J: Coupling constant, Hz: Hertz, DMSO-d6: Deuterated dimethyl sulfoxide. Delta values are expressed in ppm values.

Mass spectrometry (MS) was measured using an Agilent (ESI) mass spectrometer, model Agilent 6120B.

### Example 1. Preparation of Compounds

### 1.1. Preparation of N—((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′, 4′: 6,7]indolizino[1,2-b]quinolin-1-yl)amino)-1,6,9,12,15-pentaoxo-3-oxa-5,8,11,14-tetraazahexadecan-16-yl)-6-(2,5-dioxo-2,5-dihydro-1H-pyrrole-1-yl) hexanamide (M-01)

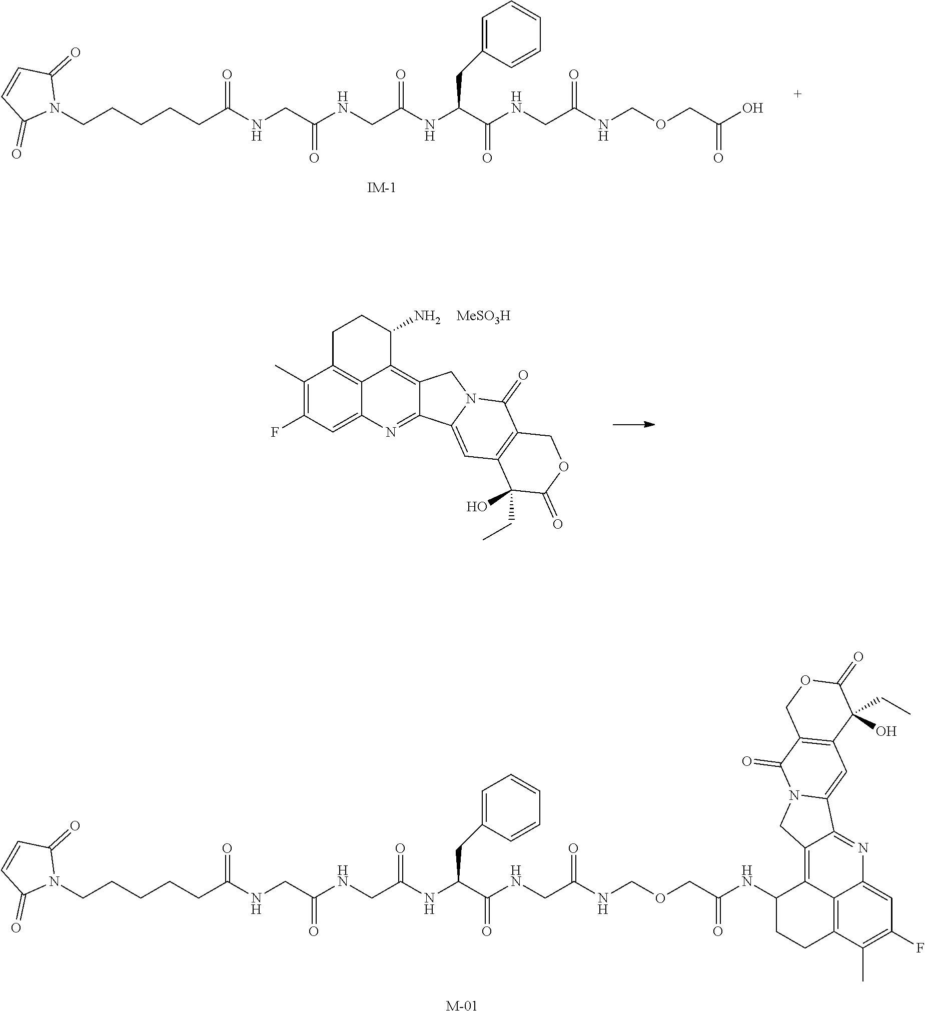

Compound IM-1 (0.40 g, 640.59 mol, its synthesis refers to patent application CN 111936169A) and exatecan mesylate (0.37 g, 704.65 mol) were dissolved in DMF (8 mL), and HATU (0.32 g, 832.77 mol) and DIPEA (0.25 g, 1.92 mmol) were added, the resulting mixture was reacted at 25° C. for a 4 hours. DIPEA was removed under reduced pressure and freeze-dried by adding water to remove most of the DMF to obtain a crude product. The crude product was purified by preparative high performance liquid chromatography (conditions were as follows) to obtain 273 mg of compound M-01.

Column: Waters XBridge Prep C18 OBD 45 mm×450 mm×8.0 μm

Mobile phase A: Acetonitrile; mobile phase B: Water (0.05% trifluoroacetic acid)

| | Mobile | Mobile | |
|---|---|---|---|
| Time | phase A | phase B | Flow rate |
| [min] | [%] | [%] | [mL/min] |
| 0.00 | 30 | 70 | 70 |
| 5.00 | 30 | 70 | 70 |
| 60.00 | 70 | 30 | 70 |

The structural representation data of M-01 were as follows:

ESI-MS (m/z): 1034.4[M+H]⁺.

### 1.2. Preparation of N—((S)-10-benzyl-1-(((1S,9S)-5-chloro-9-ethyl-9-hydroxy-4-methyl-10,13-dioxo-2,3,9,10,13,15-Hexahydro-1H,12H-benzo[de]pyrano[3′,4′: 6,7]indolizino[1,2-b]quinolin-1-yl)amino)-1,6,9,12,15-pentoxo-3-oxy-5,8,11,14-tetraazahexadecan-16-yl)-6-(2-(methylsulfonyl)pyrimidin-5-yl)hex-5-ynamide (A-05)

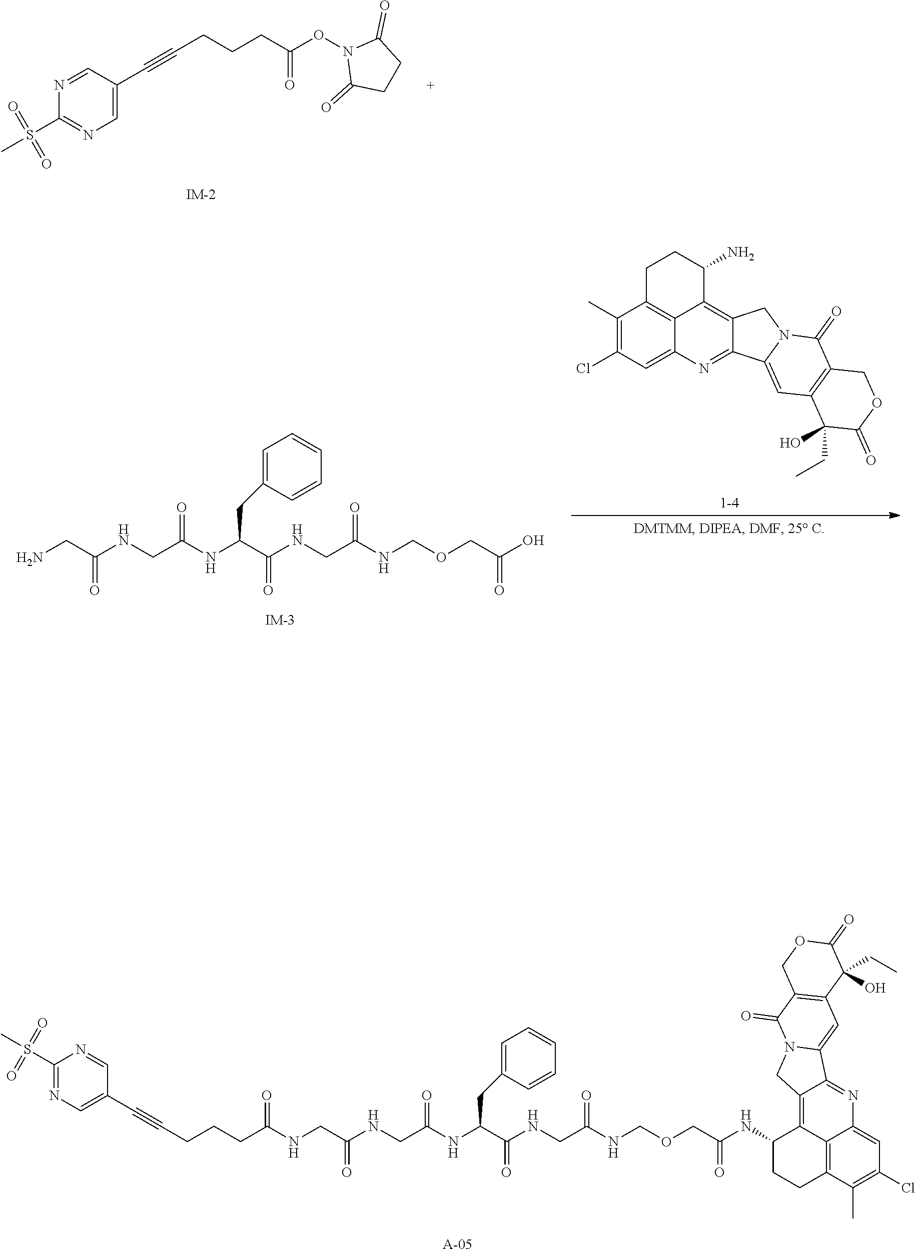

Under nitrogen protection, 2,5-dioxopyrrolidin-1-yl-6-(2-(methanesulfonyl)pyrimidin-5-yl)hex-5-ynoate (IM-2, 0.66 g, 1.80 mmol) and (R)-16-amino-10-benzyl-6,9,12,15-tetraoxo-3-oxa-5,8,11,14-tetraazahexadecanoic acid (IM-3, 0.75 g, 1.77 mmol) was added to DMF (19 mL), and the temperature was raised to 35° C. After 16 hours of reaction, (1S,9S)-1-amino-5-chloro-9-ethyl-9-hydroxy-4-methyl-1,2,3,9,12,15-hexahydro-10H,13H-benzo[de]pyran [3′, 4′: 6, 7]indolizino[1,2-b]quinoline-10,13-dione (1-4, 1.00 g, 1.77 mmol) was added to the system, cooled by the ice water to 5° C. to 15° C. DMTMM (0.98 g, 3.53 mmol) was added, and then DIPEA (1.14 g, 8.84 mmol) was drop in, at 25° C. for a 16 hour reaction time. The reaction solution was poured into a mixture of DCM (600 mL), IPA (60 mL), and water (100 mL) and stirred for 10 minutes. The DCM phase was separated, washed with brine (100 ml), concentrated to obtain crude product, and purified using preparative high-performance liquid chromatography. After purification, 0.98 g of compound A-05 was obtained by freeze-drying.

The separation and purification method of A-05 was as follows:

Column: Waters SunFire Prep C18 OBD (5 m*19 mm*150 mm)

Mobile phase A: Acetonitrile; mobile phase B: Water (0.05% formic acid)

| | Mobile | Mobile | |
|---|---|---|---|
| Time | phase A | phase B | Flow rate |
| [min] | [%] | [%] | [mL/min] |
| 0.00 | 30 | 70 | 24 |
| 2.00 | 30 | 70 | 24 |
| 18.00 | 80 | 20 | 24 |

The structural representation data of A-05 were as follows:

MS m/z (ESI): 1107.3 [M+H]+

¹H NMR (400 MHz, DMSO) δ 9.10 (s, 2H), 8.66-8.63 (m, 1H), 8.51 (d, J=8.8 Hz, 1H), 8.34-8.31 (m, 1H), 8.21-8.19 (m, 1H), 8.17-8.09 (m, 2H), 8.08-8.04 (m, 1H), 7.30 (s, 1H), 7.26-7.15 (m, 5H), 6.55 (s, 1H), 5.56-5.55 (m, 1H), 5.48-5.35 (m, 2H), 5.25-5.10 (m, 2H), 4.64 (d, J=6.4 Hz, 2H), 4.45-4.44 (m, 1H), 4.06-3.98 (m, 2H), 3.77-3.52 (m, 6H), 3.41 (s, 3H), 3.25-3.12 (m, 2H), 3.03-3.00 (m, 1H), 2.83-2.72 (m, 1H), 2.58-2.56 (m, 2H), 2.48 (s, 3H), 2.33-2.30 (m, 2H), 2.21-2.13 (m, 2H), 1.91-1.76 (m, 4H), 0.87 (t, J=7.2H z, 3H).

### 1.3. Preparation of N—((S)-10-benzyl-1-(((1S,9S)-5-fluoro-9-ethyl-9-hydroxy-4-chloro-10,13-dioxo-2,3,9,10,13,15-Hexahydro-1H,12H-benzo[de]pyrano[3′, 4′: 6,7]indolazino[1,2-b]quinolin-1-yl)amino)-1,6,9,12,15-pentoxo-3-Oxy-5,8,11,14-tetraazahexadecane-16-yl)-6-(2-(methylsulfonyl)pyrimidin-5-yl)hex-5-ynamide (A-07)

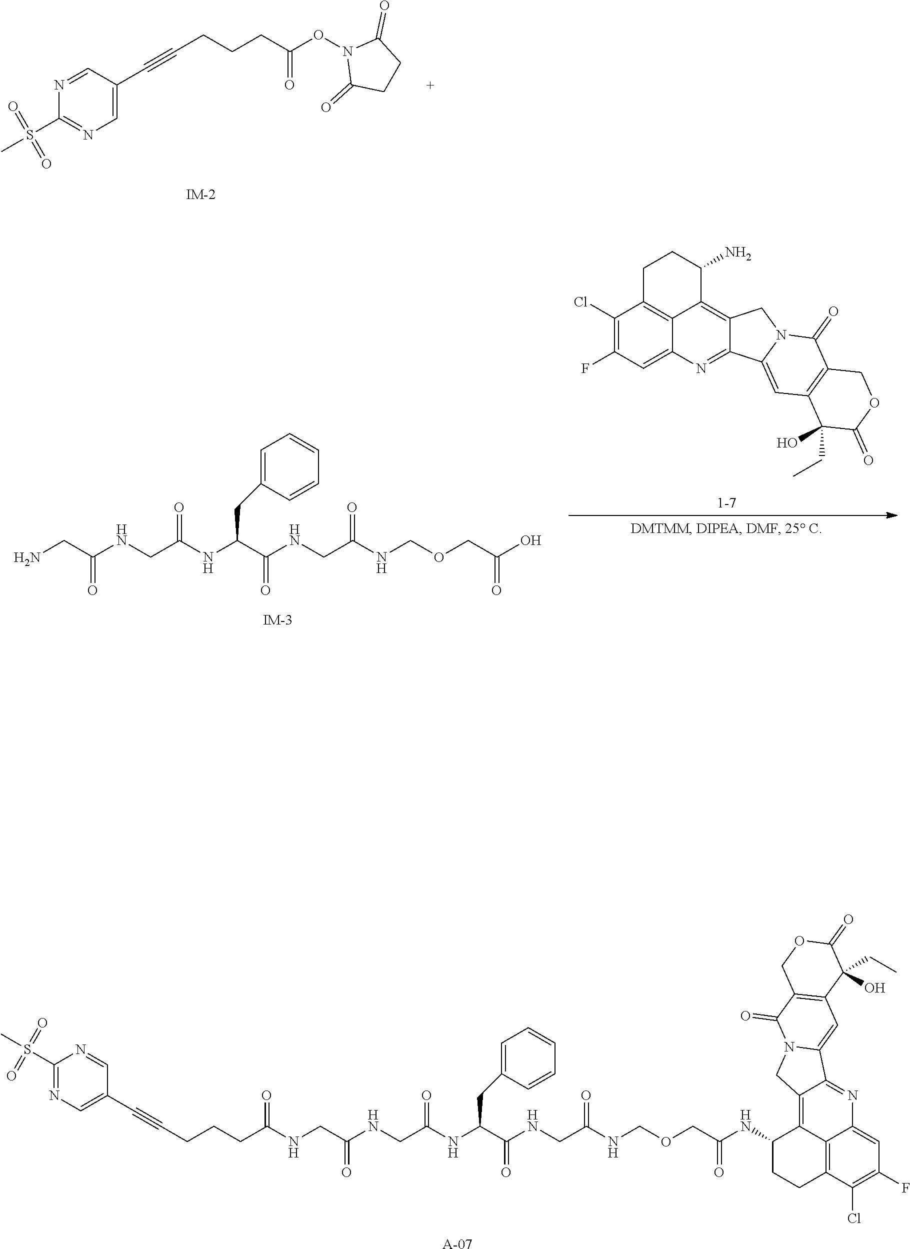

Under nitrogen protection, 2,5-dioxopyrrolidin-1-yl-6-(2-(methanesulfonyl)pyrimidin-5-yl)hex-5-ynoate (IM-2, 21.6 mg, 0.059 mmol) and (R)-16-amino-10-benzyl-6,9,12,15-tetraoxo-3-oxa-5,8,11,14-tetraazahexadecanoic acid (IM-3, 24.5 mg, 0.058 mmol) was added to DMF (1 mL), and the temperature was raised to 35° C. After 16 hours of reaction, (1S, 9S)-1-amino-5-fluoro-9-ethyl-9-hydroxy-4-chloro-1,2,3,9,12,15-hexahydro-10H,13H-benzo[de]pyrano[3′,4′:6,7]indolizino[1,2-b]quinoline-10,13-dione trifluoroacetate (30.0 mg, 0.053 mmol), HATU (30 mg, 0.079 mmol) and DIPEA (27.2 mg, 0.21 mmol) were added to the system. The reaction system was incubated at 25° C. for a 16 hour reaction time. The reaction solution was directly purified by preparative high-performance liquid chromatography and then freeze-dried to obtain 26.4 mg of compound A-07.

The separation and purification method of A-07 was as follows:

Column: SunFire Prep C18 OBD 19 mm×150 mm×5.0 μm

Mobile phase A: Acetonitrile; mobile phase B: Water (0.05% formic acid)

| | Mobile | Mobile | |
|---|---|---|---|
| Time | phase A | phase B | Flow rate |
| [min] | [%] | [%] | [mL/min] |
| 0 | 30 | 70 | 28 |
| 2 | 30 | 70 | 28 |
| 18 | 90 | 10 | 28 |

The structural representation data of A-07 were as follows:

ESI-MS (m/z): 1111.3[M+H]⁺.

### 1.4. Preparation of N-((7S,10S,13S)-1-(((1S,9S)-5-chloro-9-ethyl-9-hydroxy-4-methyl-10,13-dioxo-2,3,9,10,13,15-hexahydro-1H,12H-benzo[de]pyrano[3′, 4′: 6,7]indolizino [1,2-b]quinolin-1-yl)amino)-7,10-dimethyl-1,6,9,12-tetraoxo-3-oxa-5,8,11-triazatetradecan-13-yl)-6-(2-(methylsulfonyl) pyrimidin-5-yl)hex-5-ynamide (A-14)

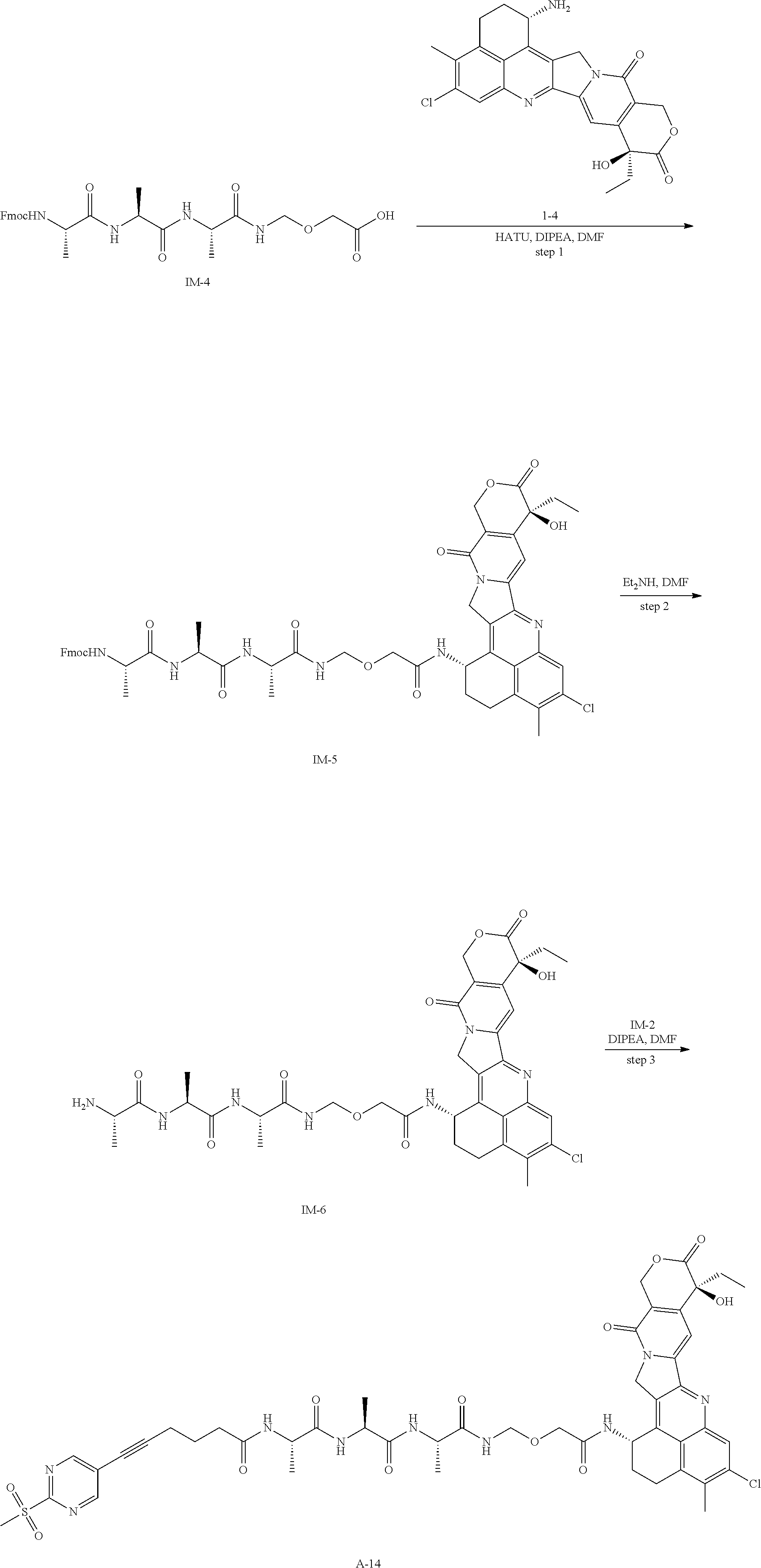

Step one: Compound IM-4 (657 mg, 1.22 mmol) and compound 1-4 (500 mg, 1.11 mmol) were dissolved in N,N-dimethylformamide (10 mL), followed by addition of HATU (630.67 mg, 1.66 mmol) and N,N-diisopropylethylamine (428 mg, 3.32 mmol), and stirred at room temperature for 1 hour. After the reaction was completed, the reaction solution was directly purified by preparative high-performance liquid chromatography and then freeze-dried to obtain 700 mg of IM-5 compound.

The preparative HPLC purification method was as follows:

Column: Waters SunFire Prep C18 OBD (5 m*19 mm*150 mm)

Mobile phase A: Acetonitrile; mobile phase B: Water (0.05% formic acid)

| | Mobile | Mobile | |
|---|---|---|---|
| Time | phase A | phase B | Flow rate |
| [min] | [%] | [%] | [mL/min] |
| 0.00 | 30 | 70 | 28 |
| 2.00 | 30 | 70 | 28 |
| 18.00 | 90 | 10 | 28 |

Step two: Compound IM-5 (500 mg, 0.513 mmol) was dissolved in N,N-dimethylformamide (2 mL), diethylamine (75.05 mg, 1.03 mmol) was added, and the reaction occurred at room temperature for 1 hour. After the reaction, the reaction solution was directly purified by preparative high-performance liquid chromatography and then freeze-dried to obtain 307 mg of IM-6 compound.

The preparative HPLC purification method was as follows:

Column: Waters SunFire Prep C18 OBD (5 m*19 mm*150 mm)

Mobile phase A: Acetonitrile; mobile phase B: Water (0.05% formic acid)

| | Mobile | Mobile | |
|---|---|---|---|
| Time | phase A | phase B | Flow rate |
| [min] | [%] | [%] | [mL/min] |
| 0.00 | 20 | 80 | 24 |
| 2.00 | 20 | 80 | 24 |
| 18.00 | 80 | 20 | 24 |

Step three: IM-6 (170 mg, 0.226 mmol) and compound IM-2 (90.83 mg, 0.249 mmol) were dissolved in N, N-dimethylformamide (10 mL) and N,N-diisopropylethylamine (29.21 mg, 0.226 mmol) was added. The reaction solution was stirred at room temperature for 16 hours. The reaction solution was directly purified by preparative high-performance liquid chromatography and then freeze-dried to obtain 50.56 mg of compound A-14.

Its structural characterization data were as follows:

MS m/z (ESI): 1002.4 [M+H]⁺

The preparative HPLC purification method was as follows:

Column: Waters SunFire Prep C18 OBD (5 m*19 mm*150 mm)

Mobile phase A: Acetonitrile; mobile phase B: Water (0.05% formic acid)

| | Mobile | Mobile | |
|---|---|---|---|
| Time | phase A | phase B | Flow rate |
| [min] | [%] | [%] | [mL/min] |
| 0.00 | 30 | 70 | 28 |
| 2.00 | 30 | 70 | 28 |
| 18.00 | 90 | 10 | 28 |

¹H NMR (400 MHz, DMSO) δ 9.11 (s, 2H), 8.68 (t, J=6.4 Hz, 1H), 8.49 (d, J=8.8 Hz, 1H), 8.16 (s, 1H), 8.10 (d, J=7.2 Hz, 1H), 8.01 (d, J=7.2 Hz, 1H), 7.91 (d, J=6.8 Hz, 1H), 7.31 (s, 1H), 6.55 (s, 1H), 5.65-5.55 (m, 1H), 5.43 (s, 2H), 5.21 (s, 2H), 4.67-4.55 (m, 2H), 4.29-4.15 (m, 3H), 3.98 (s, 2H), 3.41 (s, 3H), 3.25-3.15 (m, 2H), 2.57-2.56 (m, 2H), 2.35-2.27 (m, 2H), 2.22-2.12 (m, 2H), 1.91-1.75 (m, 4H), 1.23-1.09 (m, 9H), 0.87 (t, J=7.2 Hz, 3H).

### 1.5. Preparation of 4-((S)-2-(4-aminobutyl)-35-(4-((6-(2-(methylsulfonyl)pyrimidin-5-yl)hex-5-ynamide) methyl)-1H-1,2,3-triazol-1-yl)-4,8-dioxo-6,12,15,18,21,24,27,30,33-nonaoxa-3,9-diazapentatriacontanamido)benzyl ((S)-4-ethyl-11-(2-(N-(isopropyl)methylsulfonamido)ethyl)-3,14-dioxo-3,4,12,14-tetrahydro-1H-pyrano[3′, 4′: 6,7]indolizino[1,2-b]quinolin-4-yl) carbonate (B-01)

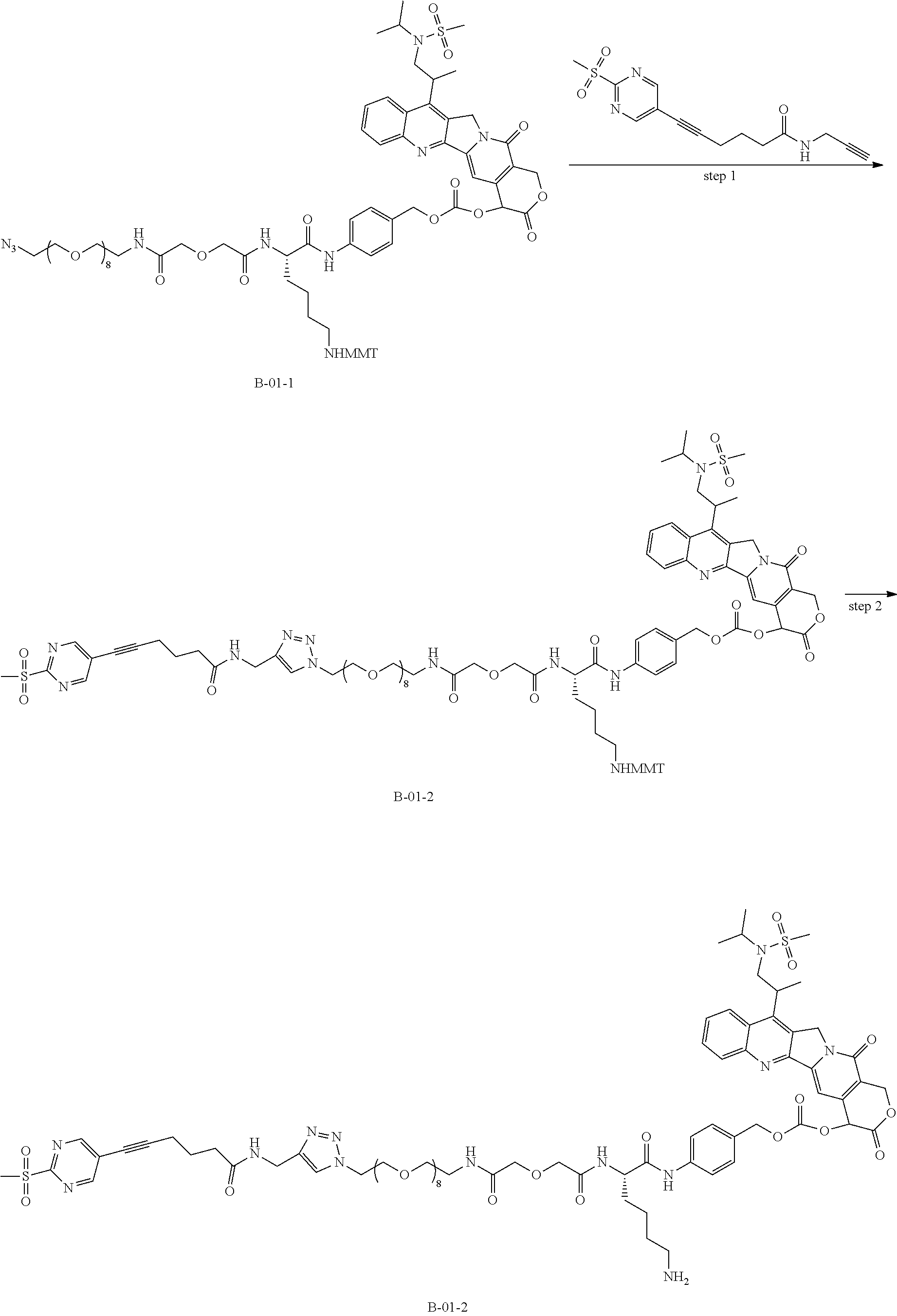

Step one: Compound B-01-1 (413.40 mg, 0.251 mmol, its synthesis refers to patent CN111295389B) was dissolved in dimethyl sulfoxide and water (2.0 mL:0.5 mL), copper bromide (72.95 mg, 0.503 mmol) and 6-(2-(methylsulfonyl)pyrimidin-5-yl)-N-(prop-2-yn-1-yl)-Hex-5-ynamide (95.10 mg, 0.302 mmol) were added, and the solution was stirred for 1 hour and then filtered. The filtrate was purified by preparative high performance liquid chromatography (conditions were as follows) to obtain 30.00 mg of compound B-01-2.

Column: SunFire Prep C18 OBD 19 mm×150 mm×5.0 μm

Mobile phase A: Acetonitrile; mobile phase B: water

| | Mobile | Mobile | |
|---|---|---|---|
| Time | phase A | phase B | Flow rate |
| [min] | [%] | [%] | [mL/min] |
| 0.00 | 40 | 60 | 28 |
| 8.00 | 40 | 60 | 28 |
| 50.00 | 90 | 10 | 28 |

Step 2: Compound B-01-2 (30.00 mg, 0.02 mmol) was dissolved in dichloromethane (1.0 mL), trifluoroacetic acid (0.2 mL) was added, and the reaction occurred at room temperature for 30 min. The reaction solution was concentrated under reduced pressure and purified by preparative high-performance liquid chromatography (conditions were as follows) to obtain 20.00 mg of the trifluoroacetate salt of compound B-01.

Column: SunFire Prep C18 OBD 19 mm×150 mm×5.0 μm

Mobile phase A: Acetonitrile; mobile phase B: Water (0.05% trifluoroacetic acid)

| | Mobile | Mobile | |
|---|---|---|---|
| Time | phase A | phase B | Flow rate |
| [min] | [%] | [%] | [mL/min] |
| 0.00 | 15 | 85 | 28 |
| 8.00 | 15 | 85 | 28 |
| 50.00 | 60 | 40 | 28 |

The structural characterization data were as follows: ESI-MS (m/z): 1631.7 [M+H]⁺, 816.0[M/2+H]⁺.

### 1.6. Preparation of 4-((S)-2-(4-aminobutyl)-35-(4-((6-(2-(methanesulfonyl)pyrimidin-5-yl)hex-5-ynamide))methyl)-1H-1,2,3-triazol-1-yl)-4,8-dioxy-6,12,15,18,21,24,27,30,33-nonaza-3,9-diazatripentadecanoyl)benzyl ((1S,9R)-9-ethyl-5-fluoro-1-(2-hydroxyacetamido)-4-methyl-10,13-dioxy-2,3,9,10,13,15-hexahydro-1H,12H-benzo [de]pyrano[3′, 4′: 6,7]indolizino[1,2-b]quinolin-9-yl)carbonate (B-02)

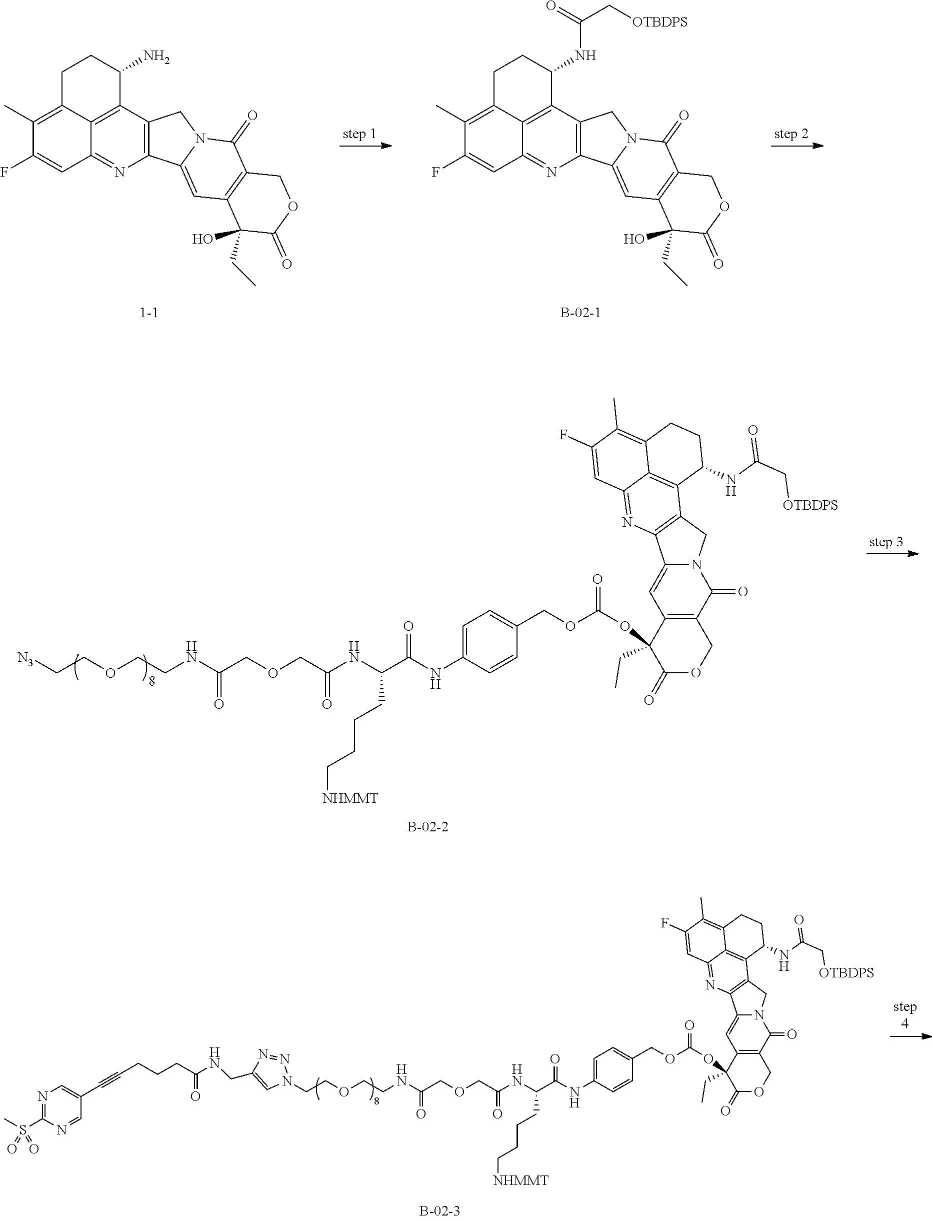

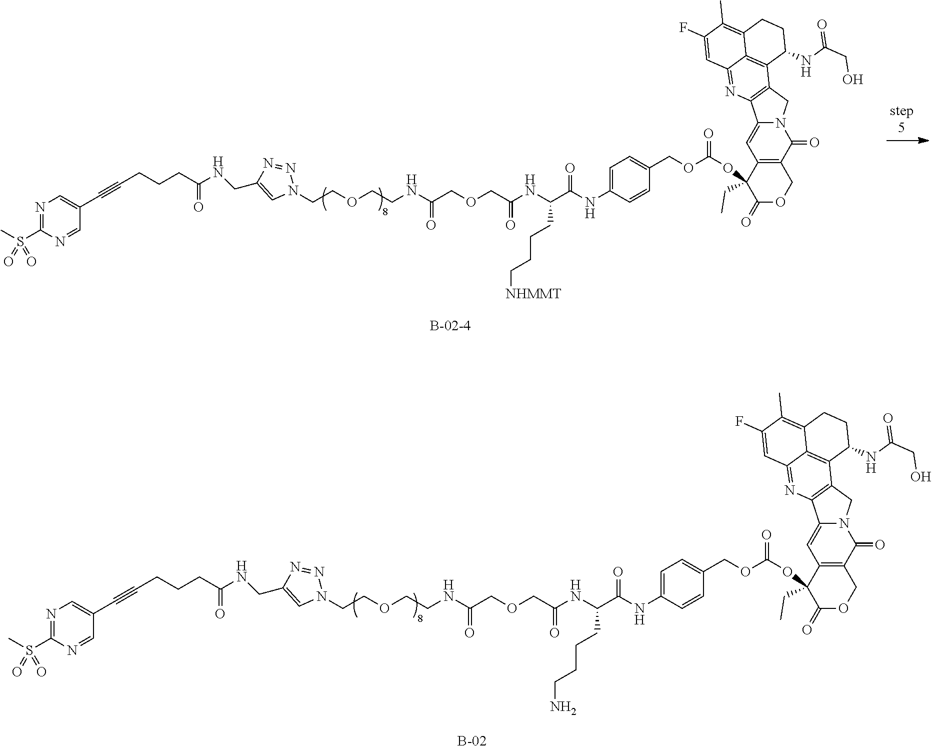

Step one: At 25° C., methanesulfonate of 1-1 (30.00 mg, 56.44 μmol) was dissolved in N,N-dimethylformamide (1 mL), and 1H-benzotriazol-1-yloxytripyrrolidinophosphonium hexafluorophosphate (58.74 mg, 112.88 μmol), N,N-diisopropylethylamine (43.76 mg, 338.63 mol) and 2-((tert-butyldiphenylsilyl)oxy)acetic acid (26.62 mg, 84.66 mol) were added in sequence, maintained at 25° C. for a reaction time of 1 hour. The reaction was monitored with liquid mass spectrometry. After the reaction was completed, water was added to the reaction solution, the reaction solution was extracted with ethyl acetate, the organic phases were combined, dried over sodium sulfate and concentrated under reduced pressure, and the crude product was separated by thin layer chromatography (dichloromethane:methanol=15:1) to obtain 27.00 mg of compound B-02-1.

Step two: At 0° C., B-02-1 (20 mg, 27.33 mol) was dissolved in dichloromethane (2 mL), and 4-dimethylaminopyridine (26.71 mg, 218.61 mol) and triphosgene (8.11 mg, 27.33 mol) in dichloromethane (0.5 mL) were added in sequence, and maintained at 0° C. for a reaction time of 0.5 hours. Residual triphosgene was replaced with nitrogen and (S)-2-(32-azido-5-oxo-3,9,12,15,18,21,24,27,30-nonaoxa-6-azadotriacontanamido)-N-(4-(hydroxymethyl)phenyl)-6-(((4-methoxyphenyl)diphenylmethyl)amino)caproamide (43.46 mg, 40.99 mol) in dichloromethane (1 mL) was added dropwise, and maintained at 0° C. for a reaction time of 0.5 hours. Liquid mass spectrometry was used to monitor the reaction. After the reaction was completed, the reaction solution was concentrated, and the crude product was separated by thin layer chromatography (dichloromethane:methanol=15:1) and purified to obtain 30.00 mg of compound B-02-2.

Step three: B-02-2 (250.00 mg, 137.51 mol) was dissolved in a mixed solvent of DMSO (2 mL) and water (0.4 mL) at 25° C., and 6-(2-(methanesulfonyl)pyrimidin-5-yl)-N-(prop-2-yn-1-yl)hex-5-ynamide (62.98 mg, 206.26 mol) and copper bromide (39.45 mg, 275.01 mol) were added, and the reaction was kept at 25° C. for 1 hour; liquid mass spectrometry chromatography was used to monitor the reaction. After the reaction was completed, the reaction solution was purified by preparative high performance liquid chromatography (conditions were as shown below), and the preparation solution was freeze-dried to obtain 150.00 mg of compound B-02-3.

Column: SunFire Prep C18 OBD 19 mm×150 mm×5.0 μm

Mobile phase A: acetonitrile; mobile phase B: water (0.05% formic acid)

| | Mobile | Mobile | |
|---|---|---|---|
| Time | phase A | phase B | Flow rate |
| [min] | [%] | [%] | [mL/min] |
| 0 | 15 | 85 | 28 |
| 18 | 90 | 10 | 28 |

Step four: B-02-3 mg, 49.45 μmol) was dissolved in tetrahydrofuran (1 mL) at 25° C., and a mixture of tetrabutylammonium fluoride (1M solution in tetrahydrofuran)/glacial acetic acid (v/v=13/1) (50 uL) was added dropwise, and the reaction was kept at 25° C. for 0.5 hours and monitored with liquid mass spectrometry. After the reaction was completed, the reaction solution was purified by preparative high-performance liquid chromatography (conditions were as follows), and the preparation solution was freeze-dried to obtain 50.00 mg of compound B-02-4.

Column: SunFire Prep C18 OBD 19 mmx 150 mmx 5.0 μm

Mobile phase A: acetonitrile; mobile phase B: water (0.05% formic acid)

| | Mobile | Mobile | |
|---|---|---|---|
| Time | phase A | phase B | Flow rate |
| [min] | [%] | [%] | [mL/min] |
| 0 | 15 | 85 | 28 |
| 20 | 90 | 10 | 28 |

Step five: B-02-4 (50 mg, 26.52 mol) was dissolved in dichloromethane (1 mL) at 25° C., trifluoroacetic acid (60.49 mg, 530.49 mol) was added, and the reaction was maintained at 25° C. for 0.5 hours. The reaction was monitored with liquid mass spectrometry. After the reaction was completed, the reaction solution was concentrated, the crude product was purified by preparative high-performance liquid chromatography (conditions were as follows), and the preparation solution was freeze-dried to obtain 23.69 mg of compound B-02.

Column: SunFire Prep C18 OBD 19 mmx 150 mmx 5.0 μm

Mobile phase A: acetonitrile; mobile phase B: water (0.05% formic acid)

| | Mobile | Mobile | |
|---|---|---|---|
| Time | phase A | phase B | Flow rate |
| [min] | [%] | [%] | [mL/min] |
| 0 | 15 | 85 | 28 |
| 18 | 90 | 10 | 28 |

The structural characterization data of B-02 were as follows:

ESI-MS (m/z): 1613.6[M+H]⁺.

### 1.7. Preparation of N-((7S,10S,13S)-1-(((1S,9S)-9-ethyl-5-fluoro-9-hydroxy-4-methyl-10,13-dioxo-2,3,9,10,13,15-Hexahydro-1H,12H-benzo[de]pyrano[3′, 4′. 6,7]indolizino[1,2-b]quinolin-1-yl)amino)-7,10,13-trimethyl-1,6,9,12,15-pentaoxo-3,17,20,23-tetraoxa-5,8,11,14-tetraazapentacosan-25-yl)-3,5-bis(2-(methylsulfonyl)pyrimidin-4-yl)benzamide (C-07)

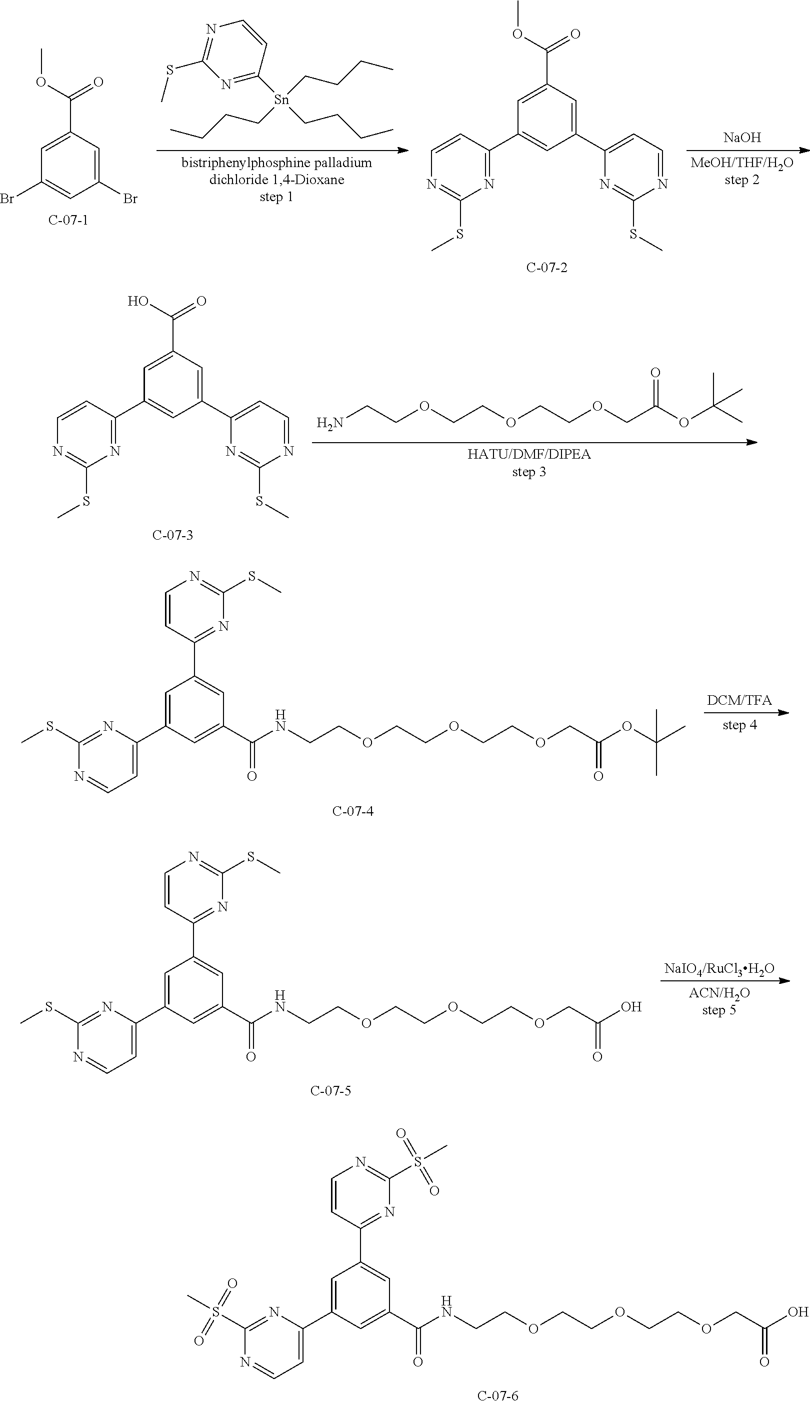

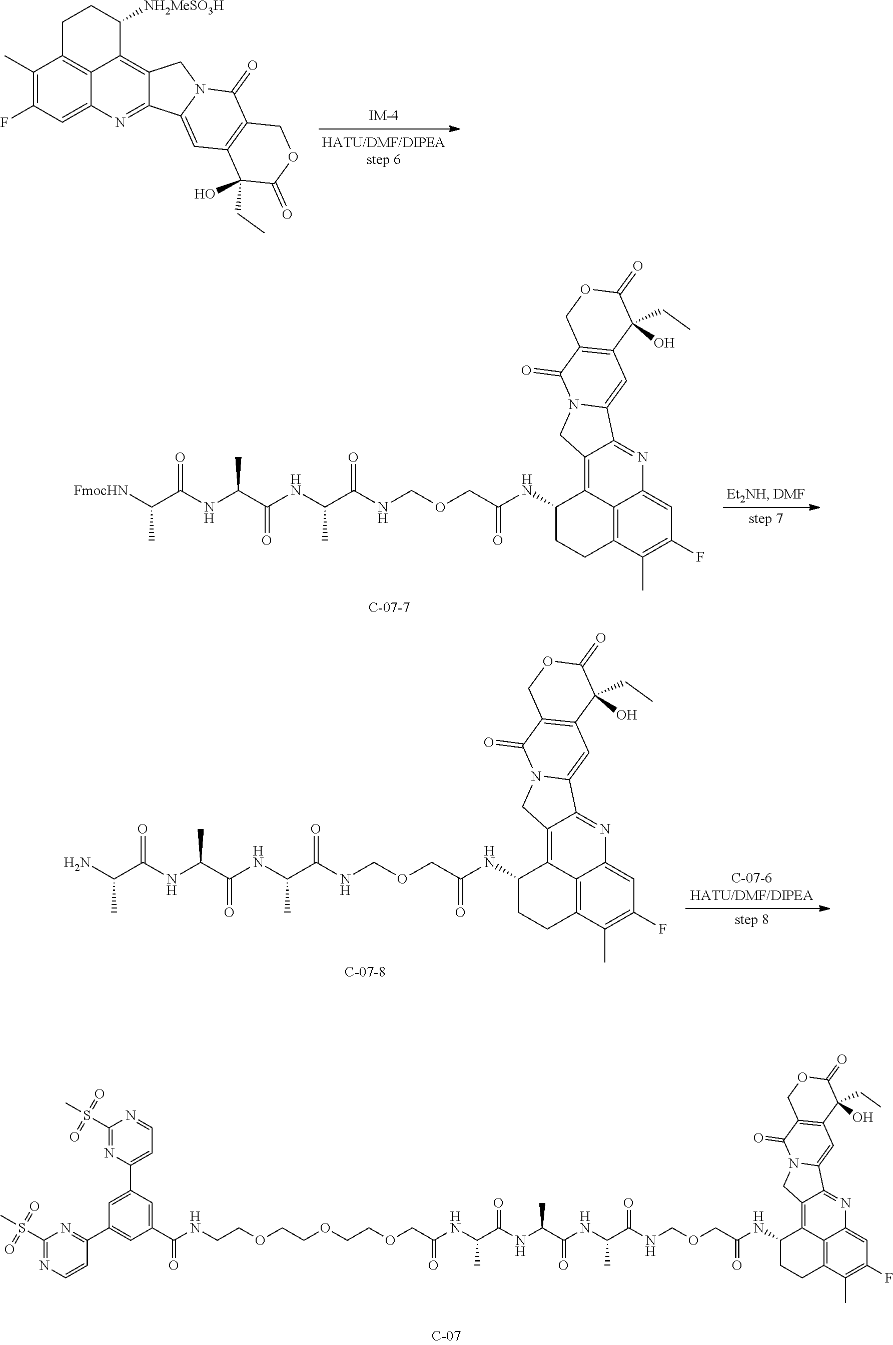

Step one: The raw materials C-07-1 (4.80 g, 16.33 mmol), tributyl(2-methylthiopyrimidin-4-yl)tin (16.27 g, 39.19 mmol) and bistriphenylphosphine palladium dichloride (2.29 g, 3.27 mmol) were dissolved in 1,4-dioxane (100 mL), and the reaction system was stirred for 5 hours at 110° C. under a nitrogen atmosphere, LC-MS was used to monitor the reaction, the reaction system was concentrated and purified through column chromatography (EA/PE=0-50%) to obtain 1.36 g of compound C-07-2.

Step two: Compound C-07-2 (510 mg, 1.33 mol), NaOH (212.24 mg, 5.31 mmol) were dissolved in THF (12.5 mL), MeOH (12.5 mL) and H₂O (2.5 mL). The reaction was stirred for 2 hours at 25° C. The reaction was monitored with LC-MS. 3N HCl was used to adjust the pH of the system to about 2. A large amount of solid precipitated. The reaction was filtered, the filter cake was collected, and dried to obtain 380 mg Compound C-07-3.

Step three: Compound C-07-3 (315 mg, 850.32 mol), tert-butyl 2-[2-(2-aminoethoxy)ethoxy]ethoxyacetate (246.31 mg, 935.35 mol), HATU (484.99 mg, 1.28 mmol), DIPEA (329.69 mg, 2.55 mmol) were added to DMF (3 mL), and reacted at 25° C. for 2 hours. The reaction was monitored by LC-MS. The reaction solution was purified by preparative high-performance liquid chromatography and then freeze-dried to obtain 40 mg of compound C-07-3. Its preparation method was as follows:

Column: Waters XBridge Prep C18OBD (5 m*19 mm*150 mm)

Mobile phase A: Acetonitrile; mobile phase B: Water (0.05% formic acid)

| | Mobile | Mobile | |
|---|---|---|---|
| Time | phase A | phase B | Flow rate |
| [min] | [%] | [%] | [mL/min] |
| 0.00 | 55 | 45 | 24 |
| 2.00 | 55 | 45 | 24 |
| 18.00 | 100 | 0 | 24 |

Step four: Compound C-07-4 (40 mg, 64.96 mol) was dissolved in DCM (3 mL) and TFA (1.5 mL), reacted at 25° C. for 1.5 hours, LC-MS was used to monitor the reaction, and the reaction system was concentrated to dryness to obtain 36 mg of compound C-07-5.

Step five: Compound C-07-5 (26 mg, 46.46 mol), sodium periodate (99.37 mg, 464.57 mol), RuCl₃·H₂O (9.64 mg, 46.46 mol) were combined and dissolved in ACN (15 mL) and water (7.5 mL), and the reaction was kept at 25° C. for 40 minutes. The reaction was monitored with LC-MS. Water and ethyl acetate were added for extraction, and the ethyl acetate layer was concentrated to obtain 28 mg of compound C-07-6.

Step six: The compound exatecan mesylate (600 mg, 1.13 mmol), (5S,8S,11S)-1-(9H-fluoren-9-yl)-5,8,11-trimethyl-3, 6, 9, 12-tetraoxy-2,15-dioxa-4,7,10,13-tetraazaheptadecan-17-oic acid (IM-4, 610.17 mg, 1.13 mmol), HATU (643.81 mg, 1.69 mmol), DIPEA (437.65 mg, 3.39 mmol) were added to DMF (6 mL), and reacted at 25° C. for 16 hours. The reaction was monitored by LC-MS. Water was added to the reaction solution to precipitate a large amount of solid, which was filtered and collected. The solid was dissolved in DCM and concentrated to obtain a crude product, which was purified by column chromatography (DCM/MeOH=0-10%) to obtain 660 mg of compound C-07-7.

Step 7: Compound C-07-7 (660 mg, 688.94 mol) was dissolved in N,N-dimethylformamide (6 mL), diethylamine (251.94 mg, 3.44 mmol) was added, and the reaction was carried out at room temperature for 1 hour. After the reaction, the reaction solution was directly purified by preparative high-performance liquid chromatography and then freeze-dried to obtain 325 mg of compound C-07-8. Its preparation method was as follows:

Column: Waters SunFire Prep C18 OBD (5 m*19 mm*150 mm)

Mobile phase A: Acetonitrile; mobile phase B: Water (0.05% formic acid)

| | Mobile | Mobile | |
|---|---|---|---|
| Time | phase A | phase B | Flow rate |
| [min] | [%] | [%] | [mL/min] |
| 0.00 | 10 | 90 | 28 |
| 4.00 | 10 | 90 | 28 |
| 20.00 | 90 | 10 | 28 |

Step eight: Compounds C-07-6 (15.95 mg, 25.58 mol), C-07-8 (20 mg, 25.58 mol), HATU (14.59 mg, 38.37 mol), and DIPEA (9.92 mg, 76.75 mol) were added to DMF (3 mL), and the reaction was kept at 25° C. for 2 h. The reaction was monitored by LC-MS. The reaction solution was purified by preparative high-performance liquid chromatography and then freeze-dried to obtain 7 mg of compound C-07.

Its structural characterization data were as follows:

ESI-MS (m/z): 1342.4 [M+H]⁺.

Its preparation method was as follows:

Column: Waters XBridge Prep C18OBD (5 m*19 mm*150 mm)

Mobile phase A: Acetonitrile; mobile phase B: Water (0.05% formic acid)

| | Mobile | Mobile | |
|---|---|---|---|
| Time | phase A | phase B | Flow rate |
| [min] | [%] | [%] | [mL/min] |
| 0.00 | 20 | 80 | 24 |
| 2 | 20 | 80 | 24 |
| 18.00 | 90 | 10 | 24 |

### 1.8. Preparation of N-((7S,10S,13S)-1-(((1S,9S)-9-ethyl-5-fluoro-9-hydroxy-4-methyl-10,13-dioxo-2,3,9,10,13,15-Hexahydro-1H,12H-benzo[de]pyrano[3′, 4′: 6,7]indolizino[1,2-b]quinolin-1-yl)amino)-7,10,13-trimethyl-1,6,9,12,15-pentaoxo-3,17,20,23-tetraoxa-5,8,11,14-tetraazapentacosan-25-yl)-3,5-bis(2-(methylsulfonyl)pyrimidin-5-yl)benzamide (C-10)

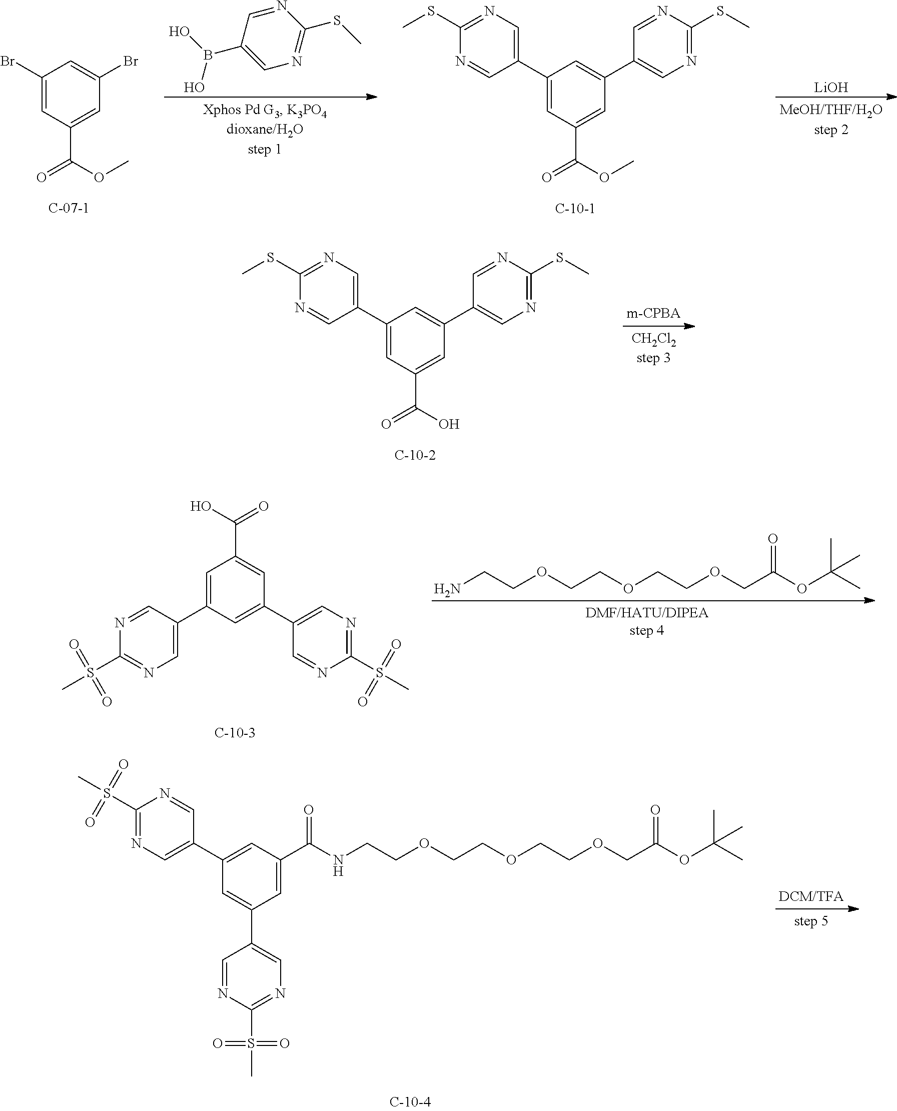

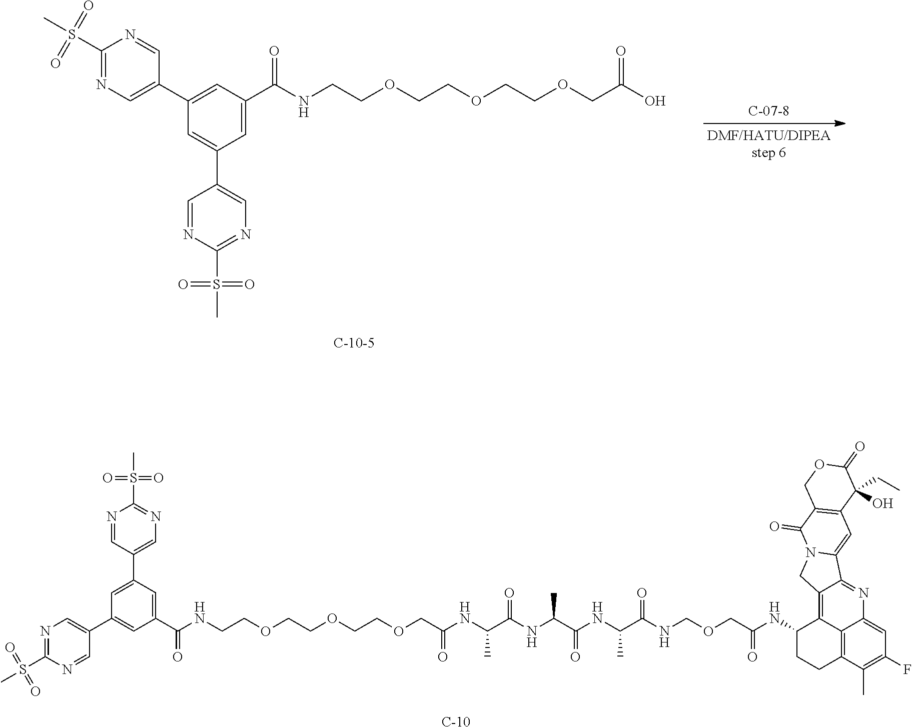

Step one: Raw materials C-07-1 (720 mg, 2.45 mmol), 2-methylthiopyrimidine-5-boronic acid (874 mg, 5.14 mmol), XPhosPd G3 (207 mg, 245 mol), K₃PO₄ (1.56 g, 7.35 mmol) were combined and added to a mixed solvent of 1,4-dioxane (12 mL) and H₂O (4 mL), the reaction system was stirred at 90° C. for 3 hours under a nitrogen atmosphere. The reaction was monitored with LC-MS, filtered through diatomaceous earth, and water and ethyl acetate were added to the filtrate, which was extracted and concentrated to obtain a crude product, which was purified by column chromatography (EA/PE=0-25%) to obtain 710 mg of compound C-10-1.

Step two: Compound C-10-1 (650 mg, 1.69 mol) and lithium hydroxide (121 mg, 5.07 mmol) were dissolved in THE (2 mL), MeOH (2 mL) and H₂O (2 mL). The reaction was stirred for 2 hours at 25° C. The reaction was monitored with LC-MS. 1N HCl was used to adjust the pH of the system to about 2. A large amount of solid precipitated. The precipitate was filtered, and the filter cake was collected and dried to obtain 560 mg of compound C-10-2.

Step three: compound C-10-2 (450.80 mg, 1.22 mmol) was dissolved in DCM (10 mL), m-CPBA (2.46 g, 12.1 mmol, purity 85%) was added to the reaction system, and the reaction was maintained at 25° C. for 12 hours and monitored with LC-MS. The solvent was dried by nitrogen flow to obtain a crude product, which was purified by preparative high-performance liquid chromatography and then freeze-dried to obtain 153 mg of compound C-10-3.

Its preparation method was as follows:

Column: Phenomenex Luna C18 200*40 mm*10 um.

Mobile phase A: Acetonitrile; mobile phase B: Water (0.05% hydrochloric acid)

Mobile phase: [water(HCl)-ACN]; B %: 13%-43%, 10 min).

| | Mobile | Mobile | |
|---|---|---|---|
| Time | phase A | phase B | Flow rate |
| [min] | [%] | [%] | [mL/min] |
| 0.00 | 13 | 87 | 25 |
| 10.00 | 43 | 57 | 25 |
| 18.00 | 90 | 10 | 25 |

Step four: Compound C-10-3 (140 mg, 322.25 mol), tert-butyl 2-[2-(2-aminoethoxy)ethoxy]ethoxyacetate (84.86 mg, 322.25 mol), HATU (183.80 mg, 483.37 mol), DIPEA (124.94 mg, 966.75 mol) were added to DMF (4 mL), and reacted at 25° C. for 2 hours. The reaction was monitored by LC-MS. The reaction solution was purified by preparative high-performance liquid chromatography and then freeze-dried to obtain 51 mg of compound C-10-4.

Its preparation method was as follows:

Column: Waters XBridge Prep C18OBD (5 m*19 mm*150 mm)

Mobile phase A: Acetonitrile; mobile phase B: Water (0.05% formic acid)

| Time | Mobile | Mobile | Flow rate |
|---|---|---|---|
| [min] | phase A [%] | phase B [%] | [mL/min] |
| 0.00 | 30 | 70 | 24 |
| 2.00 | 30 | 70 | 24 |
| 18.00 | 90 | 10 | 24 |

Step five: Compound C-10-4 (50 mg, 73.56 mol) was added to DCM (2 mL) and TFA (1 mL), reacted at 25° C. for 1 hour, monitored using LC-MS, and the reaction system was concentrated to dryness to obtain 45 mg of compound C-10-5.

Step six: Compounds C-10-5 (31.91 mg, 51.17 mol), C-07-8 (40 mg, 51.17 mol), HATU (29.18 mg, 76.75 mol), DIPEA (19.84 mg, 153.50 mol) were added to DMF (3 mL), and the reaction was maintained at 25° C. for 2 h. The reaction was monitored by LC-MS. The reaction solution was purified by preparative high-performance liquid chromatography and then freeze-dried to obtain 13 mg of compound C-10.

Its structural characterization data were as follows:

ESI-MS (m/z): 1342.5 [M+H]⁺.

Its preparation method was as follows:

Column: Waters XBridge Prep C18OBD (5 m*19 mm*150 mm)

Mobile phase A: Acetonitrile; mobile phase B: Water (0.05% formic acid)

| Time | Mobile | Mobile | Flow rate |
|---|---|---|---|
| [min] | phase A [%] | phase B [%] | [mL/min] |
| 0.00 | 20 | 80 | 24 |
| 2.00 | 20 | 80 | 24 |
| 18.00 | 90 | 10 | 24 |

### 1.9. Preparation of N-((7S,10S,13S)-1-(((1S,9S)-9-ethyl-5-chloro-9-hydroxy-4-methyl-10,13-dioxo-2,3,9,10,13,15-Hexahydro-1H,12H-benzo[de]pyrano[3′, 4′: 6,7]indolizino[1,2-b]quinolin-1-yl)amino)-7,10,13-trimethyl-1,6,9,12,15-pentaoxo-3,18,21,24-tetraoxa-5,8,11,14-tetraazahexacosan-26-yl)-3,5-bis(2-(methylsulfonyl)pyrimidin-5-yl)benzamide (C-17)

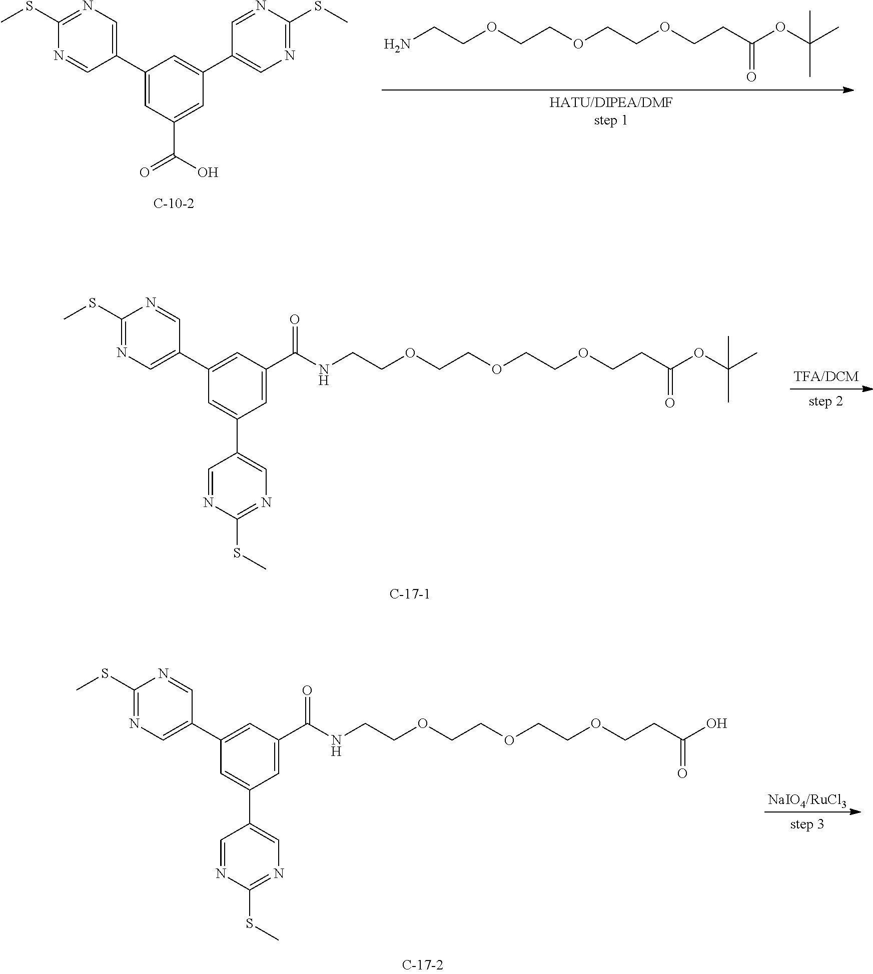

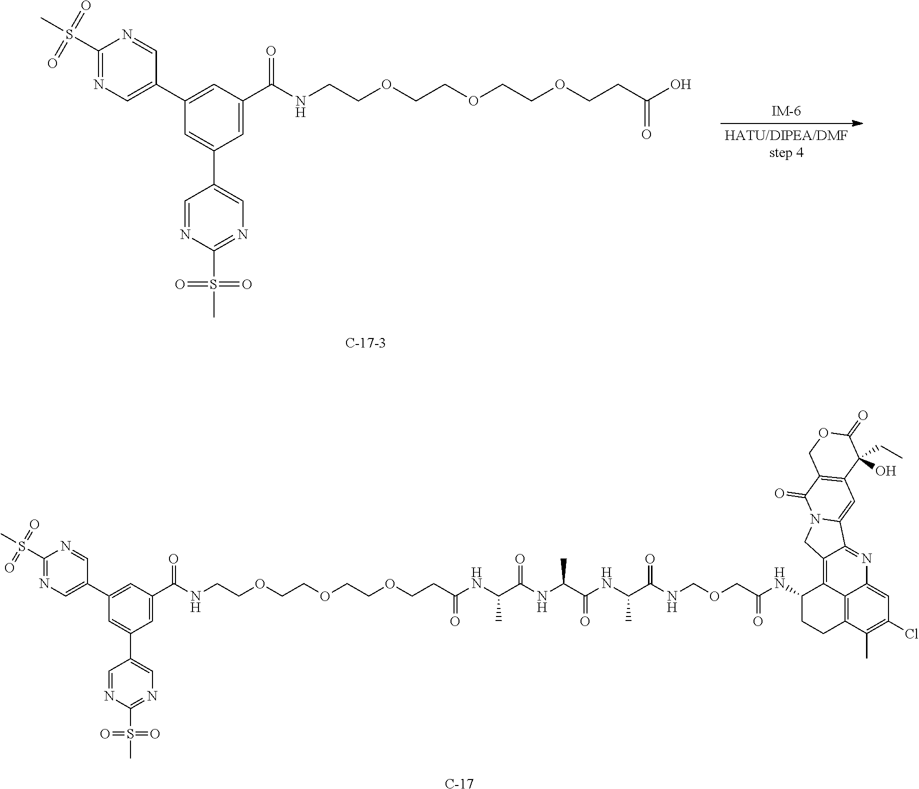

Step one: C-10-2 (3.00 g, 8.10 mmol) and tert-butyl 3-[2-[2-(2-aminoethoxy)ethoxy]ethoxy]-propionate (2.25 g, 8.10 mmol) were added to DMF (3 mL), followed by HOBt (3.28 g, 24.3 mmol), EDCI (4.66 g, 24.3 mmol) and DIPEA (4.19 g, 32.4 mmol, 5.64 mL), and the temperature was raised to 60° C. for 2 hours. Water (50 mL) was added to the reaction solution, extracted with ethyl acetate (30 mL×3), the combined organic phases were dried over anhydrous sodium sulfate, filtered and concentrated to obtain crude C-17-1 (3.8 g, 4.75 mmol), which was used directly in the next step without purification.

Step two: C-17-1 (3.40 g, 5.40 mmol) was dissolved in dichloromethane (30 mL), and trifluoroacetic acid (10.8 g, 94.2 mmol, 7 mL) was added. The reaction solution was stirred at 25° C. for 2 hours. The reaction solution was directly concentrated, purified by preparative high-performance liquid chromatography, and then freeze-dried to obtain C-17-2 (2.09 g, 3.64 mmol).

Its preparation method was as follows:

Column: Phenomenex luna C18 (250 mm*70 mm*10 m)

Mobile phase A: Acetonitrile; mobile phase B: Water (0.05% formic acid)

| Time | Mobile | Mobile | Flow rate |
|---|---|---|---|
| [min] | phase A [%] | phase B [%] | [mL/min] |
| 0.00 | 30 | 70 | 30 |
| 22.00 | 60 | 40 | 30 |

Step three: C-17-2 (56 mg, 97.61 μmol) was added to acetonitrile (6 mL) and water (3 mL), and then sodium periodate (208.79 mg, 976.15 μmol) and ruthenium trichloride hydrate (8.10 mg, 39.05 μmol) were added to the reaction system, which was stirred at 25° C. for 30 minutes and monitored with LC-MS. Water and ethyl acetate were added for extraction and concentrated to obtain C-17-3 (60 mg).

Step four: IM-6 (20 mg, 25.06 μmol), C-17-3 (16 mg, 25.06 μmol), HATU (19.05 mg, 50.11 μmol), DIPEA (16.19 mg, 125.28 μmol) were added to DMF (3 mL) in sequence, and the reaction system was adjusted to 25° C. and maintained for 1 hour. The reaction solution was directly purified by preparative high-performance liquid chromatography and then freeze-dried to obtain C-17 (16 mg).

Its structural characterization data were as follows:

ESI-MS (m/z): 1371.4 [M+H]⁺.

Its preparation method was as follows:

Column: Waters XBridge Prep C18OBD (5 m*19 mm*150 mm)

Mobile phase A: Acetonitrile; mobile phase B: Water (0.05% formic acid)

| Time | Mobile | Mobile | Flow rate |
|---|---|---|---|
| [min] | phase A [%] | phase B [%] | [mL/min] |
| 0.00 | 20 | 80 | 30 |
| 2.00 | 20 | 80 | 30 |
| 18.00 | 80 | 20 | 30 |

### 1.10. Preparation of N-((7S,10S,13S)-1-(((1S,9S)-5-chloro-9-ethyl-9-hydroxy-4-methyl-10,13-dioxo-2,3,9,10,13,15-Hexahydro-1H,12H-benzo[de]pyrano[3′, 4′: 6,7]indolizino[1,2-b]quinolin-1-yl)amino)-7,10,13-trimethyl-1,6,9,12,15-pentaoxo-3,18,21,24-tetraoxa-5,8,11,14-tetraazahexacosane-26-yl)-2,6-bis(2-(methylsulfonyl)pyrimidin-5-yl)isonicotinamide (C-19)

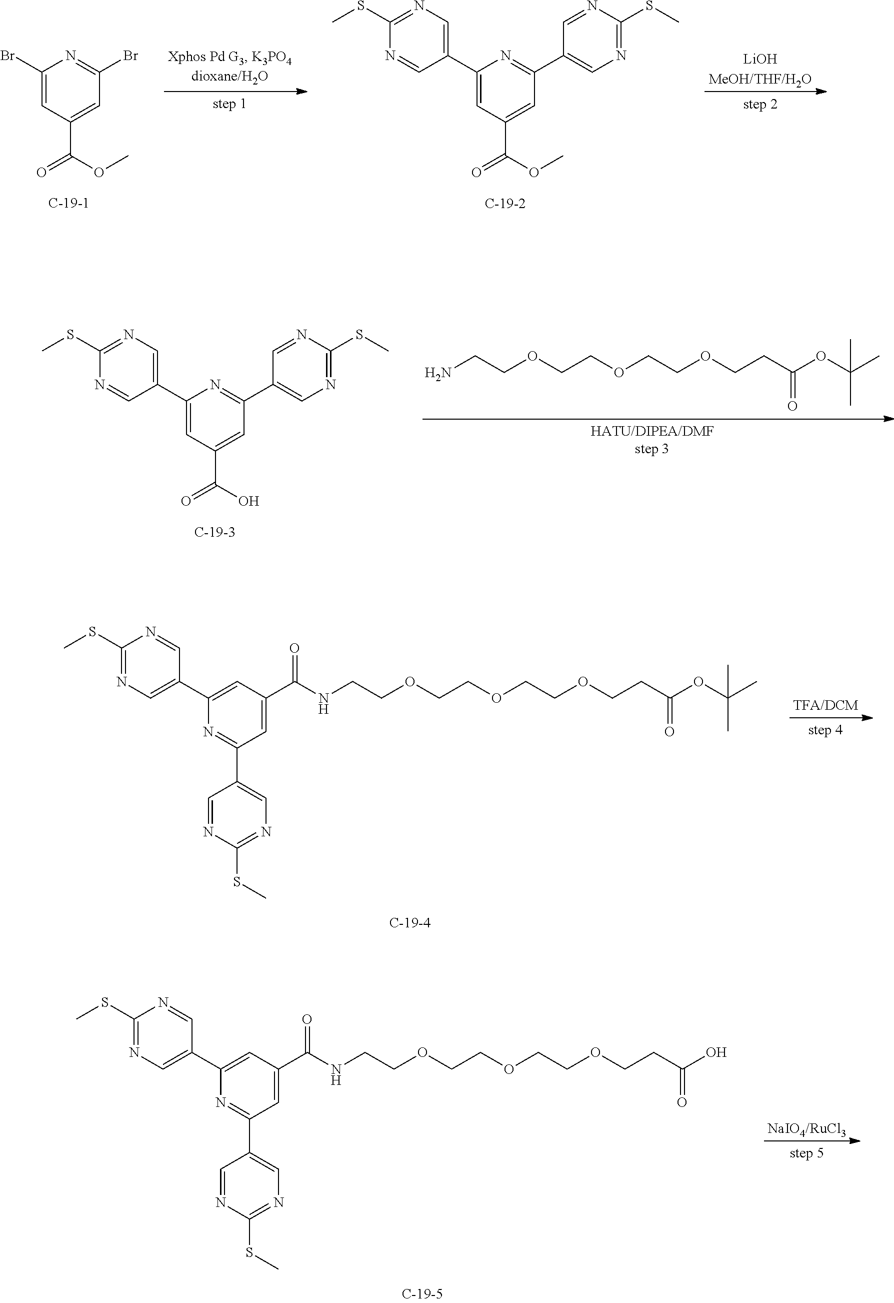

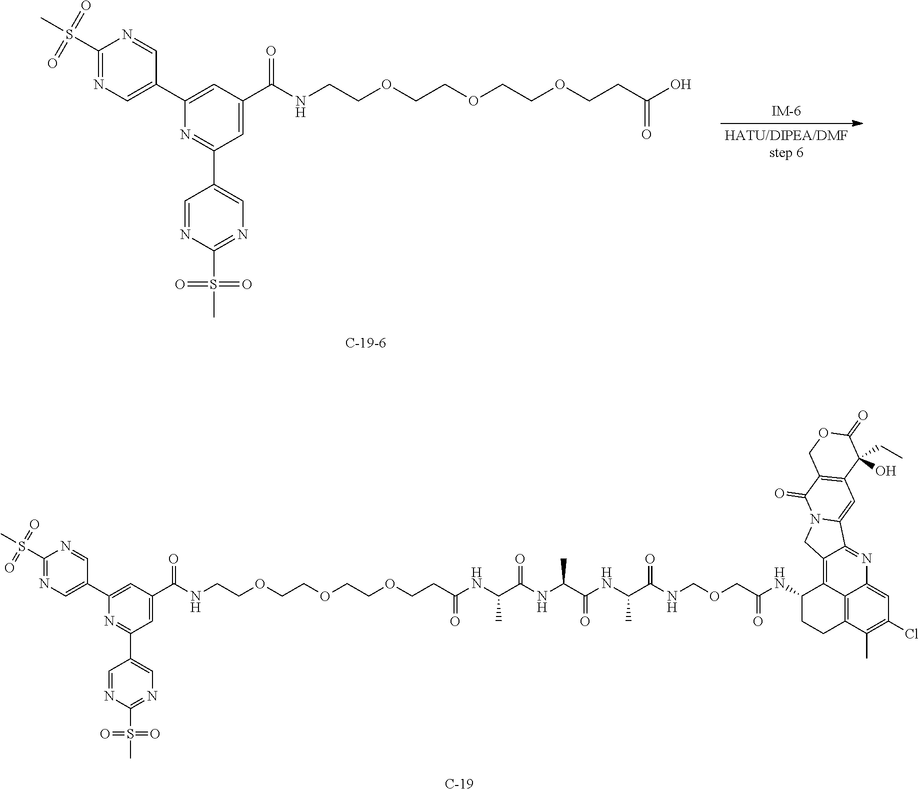

Step one: C-19-1 (5.00 g, 16.9 mmol), (2-(methylthio)pyrimidin-5-yl)boronic acid (6.34 g, 37.3 mmol), XPhos Pd G3 (1.44 g, 1.70 mmol), and potassium phosphate (10.80 g, 50.9 mmol) were combined and added to 1,4-dioxane (51.0 mL) and water (17.0 mL). The reaction system was replaced with nitrogen three times and reacted at 100° C. for 5 hours. After the reaction system was cooled to room temperature, water (50.0 mL) was added to the reaction solution and filtered. The filtrate was concentrated to obtain a crude product, which was beaten with petroleum ether and filtered again. The filter cake was vacuum dried to obtain C-19-2 (5.65 g).

Step two: C-19-2 (5.26 g, 13.7 mmol) was dissolved in THE (30.0 mL), MeOH (30.0 mL) and water (30.0 mL), LiOH·H₂O (1.72 g, 40.9 mmol) was added, and the reaction was stirred at 25° C. for 2 hours. The reaction solution was adjusted to pH 3 with 1 N hydrochloric acid aqueous solution. A solid precipitated and was filtered, and the filter cake was vacuum dried to obtain C-19-3 (4.20 g).

Step three: C-19-3 (1.50 g, 4.04 mmol) and tert-butyl 3-(2-(2-aminoethoxy)ethoxyethoxy)propionate (1.12 g, 4.04 mmol) were dissolved in DMF (20.0 mL), HOBt (1.64 g, 12.1 mmol), EDCI (2.32 g, 12.1 mmol) and DIPEA (2.09 g, 16.2 mmol) were added in sequence, the temperature was raised to 60° C. and stirred for 2 hours. The reaction system was allowed to cool to room temperature, water (10.0 mL) and ethyl acetate (20.0 mL) were added to the reaction solution, the aqueous phase was extracted twice with ethyl acetate (25.0 mL*2), and the organic phases were combined. The reaction was dried over anhydrous sodium sulfate, filtered, and concentrated to obtain C-19-4 crude product (2.50 g), which was used directly in the next step without purification.

Step four: C-19-4 (2.50 g, 3.96 mmol) was dissolved in dichloromethane (3.00 mL). After adding TFA (4.61 g, 40.4 mmol), the reaction system was stirred at 25° C. for 12 hours. The reaction solution was directly concentrated, purified by preparative high-performance liquid chromatography, and then freeze-dried to obtain C-19-5 (1.20 g).

Its preparation method was as follows:

Column: Phenomenex luna C18 (150 mm*25 mm*10 μm)

Mobile phase A: Acetonitrile; mobile phase B: Water (0.05% formic acid)

| Time | Mobile | Mobile | Flow rate |
|---|---|---|---|
| [min] | phase A [%] | phase B [%] | [mL/min] |
| 0.00 | 36 | 64 | 30 |
| 15.00 | 66 | 34 | 30 |

Step five: C-19-5 (1.10 g, 1.91 mmol) was dissolved in a mixed solvent of acetonitrile (30 mL) and water (15 mL), ruthenium trichloride hydrate (39.70 mg, 0.19 mmol) and sodium periodate (4.09 g, 19.14 mmol) were added, and the reaction system was reacted at 25° C. for 1 hour and then extracted with water (50 mL) and ethyl acetate (80 mL). The organic phase was concentrated to obtain a crude product, which was purified by column chromatography (MeOH/DCM=10.20%), and concentrated to obtain C-19-6 (130 mg).

Step six: IM-6 (20.0 mg, 0.025 mmol) and C-19-6 (16.0 mg, 0.025 mmol) were added to DMF (1 mL) and stirred to dissolve. HATU (19.0 mg, 0.050 mmol) and DIPEA (12.9 mg, 0.100 mmol) were added, the reaction was maintained at room temperature for 2 hours, and the reaction solution was directly purified by preparative high-performance liquid chromatography and then freeze-dried to obtain C-19 (20.4 mg).

Its structural characterization data were as follows:

ESI-MS (m/z):1372.4 [M+H]⁺.

Column: Waters XBridge Prep C18OBD (5 m*19 mm*150 mm)

Mobile phase A: Acetonitrile; mobile phase B: Water (0.05% formic acid)

| Time | Mobile | Mobile | Flow rate |
|---|---|---|---|
| [min] | phase A [%] | phase B [%] | [mL/min] |
| 0.00 | 30 | 70 | 30 |
| 4.00 | 30 | 70 | 30 |
| 24.00 | 90 | 10 | 30 |

### Preparation of N-((7S,10S,13S)-1-(((1S,9S)-5-chloro-9-ethyl-9-hydroxy-4-methyl-10,13-dioxo-2,3,9,10,13,15-Hexahydro-1H,12H-benzo[de]pyrano[3′, 4′: 6,7]indolizino[1,2-b]quinolin-1-yl)amino)-7,10,13-trimethyl-1,6,9,12,15-pentaoxo-3,18,21,24,27,30,33,36,39-nonaoxa-5,8,11,14-tetraazahentetracontan-41-yl)-3,5-bis(2-(methylsulfonyl)pyrimidin-5-yl)benzamide (C-21)

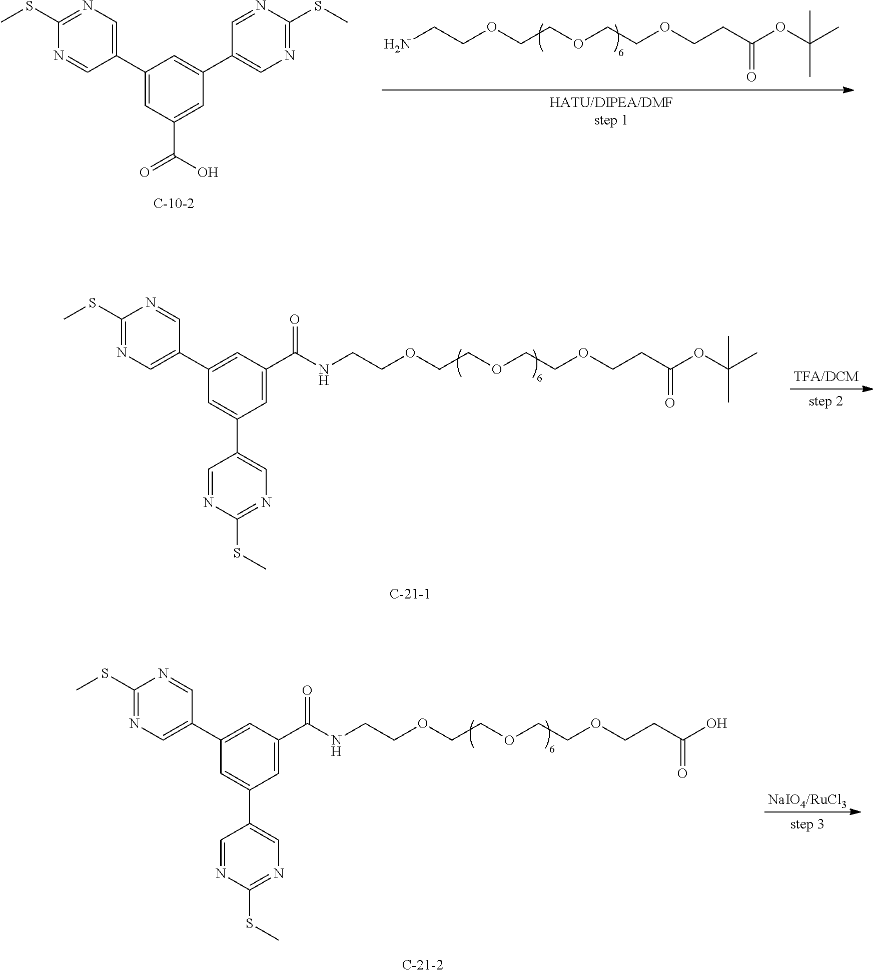

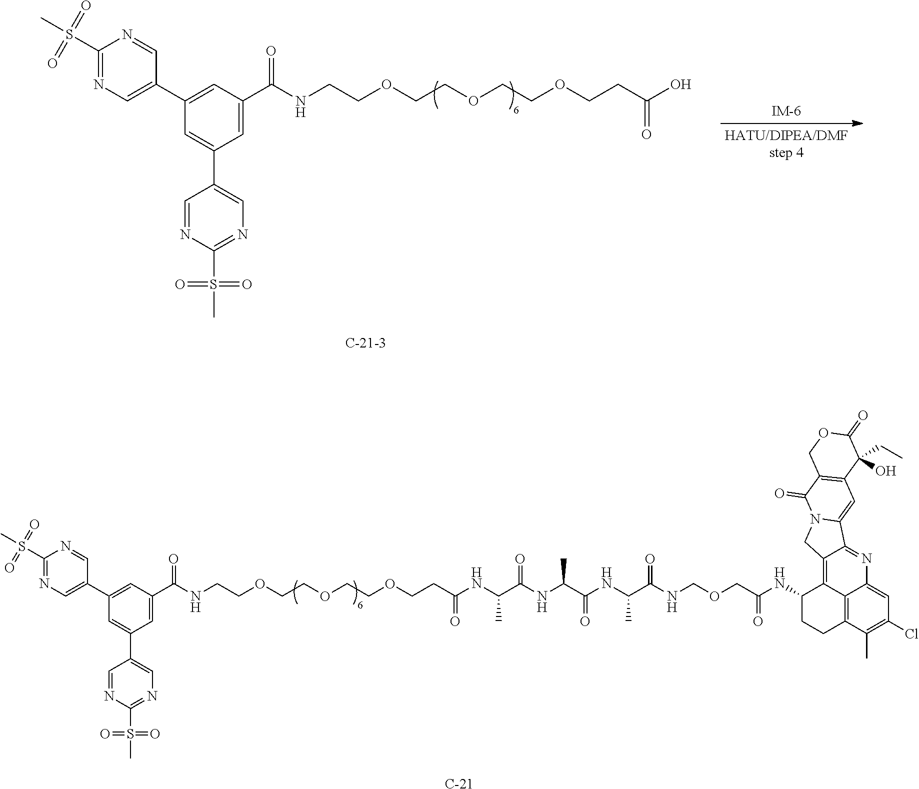

Step one: C-10-2 (3.00 g, 8.10 mmol) and tert-butyl 1-amino-3,6,9,12,15,18,21,24-octaoxaheptacosan-27-ate (4.03 g, 8.10 mmol) were added to DMF (40 mL), sequentially HOBt (3.28 g, 24.3 mmol), EDCI (4.66 g, 24.3 mmol) and DIPEA (4.19 g, 32.4 mmol, 5.64 mL) were added, and the reaction system was stirred at 60° C. for 2 hours. Water (100 mL) and ethyl acetate were added to the reaction solution for extraction (60 mL×3). The organic phases were combined, dried over anhydrous sodium sulfate, filtered, and concentrated to obtain C-21-1 (4.20 g, 4.14 mmol). It was used directly in the next step without purification.

Step two: C-21-1 (3.60 g, 4.24 mmol) was dissolved in dichloromethane (30 mL), TFA (15.3 g, 134 mmol, 10 mL) was added, and the reaction system was stirred at 25° C. for 6 hours. Water (60 mL) and ethyl acetate were added to the reaction solution for extraction (40 mL×3). The organic phases were combined, dried over anhydrous sodium sulfate, filtered, and concentrated to obtain a crude product. The crude product was purified by preparative high-performance liquid chromatography and then freeze-dried to obtain C-21-2 (2.93 g, 3.63 mmol).

Its preparation method was as follows:

Column: Phenomenex luna C18 (250 mm*70 mm*10 μm)

Mobile phase A: Acetonitrile; mobile phase B: Water (0.05% formic acid)

| Time | Mobile | Mobile | Flow rate |
|---|---|---|---|
| [min] | phase A [%] | phase B [%] | [mL/min] |
| 0.00 | 30 | 70 | 30 |
| 15.00 | 60 | 40 | 30 |

Step three: C-21-2 (148 mg, 0.186 mmol) was added to acetonitrile (15 mL) and water (7.5 mL), and then sodium periodate (398.71 mg, 1.86 mmol) and ruthenium trichloride hydrate (15.47 mg, 74.56 μmol) were added to the reaction system, and the reaction was stirred at 25° C. for 30 minutes. The reaction system was extracted with water and ethyl acetate and concentrated to obtain C-21-3 (155 mg).

Step four: IM-6 (27.91 mg, 34.97 μmol), C-21-3 (30 mg, 34.97 μmol), HATU (26.59 mg, 69.93 μmol), and DIPEA (22.60 mg, 174.84 μmol) were combined and added to DMF (3 mL), and the reaction system was reacted at 25° C. for 1 hr. The reaction solution was purified by high performance liquid chromatography and then freeze-dried to obtain C-21 (15 mg).

Its structural characterization data were as follows:

ESI-MS (m/z): 1591.7 [M+H]⁺.

Its preparation method was as follows:

Column: Waters XBridge Prep C18OBD (5 μm*19 mm*150 mm)

Mobile phase A: Acetonitrile; mobile phase B: Water (0.05% formic acid)

| Time | Mobile | Mobile | Flow rate |
|---|---|---|---|
| [min] | phase A [%] | phase B [%] | [mL/min] |
| 0.00 | 20 | 80 | 30 |
| 2.00 | 20 | 80 | 30 |
| 18.00 | 90 | 10 | 30 |

### 1.12. Preparation of N-((7S,10S,13S)-1-(((1S,9S)-5-chloro-9-ethyl-9-hydroxy-4-methyl-10,13-dioxo-2,3,9,10,13,15-Hexahydro-1H,12H-benzo[de]pyrano[3′, 4′: 6,7]indolizino [1,2-b]quinolin-1-yl)amino)-7, 10,13-Trimethyl-, 6,9,12,15-Pentaoxo-3,18,21,24,27,30,33,36,39-nonoxa-5,8,11,14-tetraazahentetracontan-41-yl)-2,6-bis(2-(methyl sulfonyl)pyrimidin-5-yl)isonicotinamide (C-23)

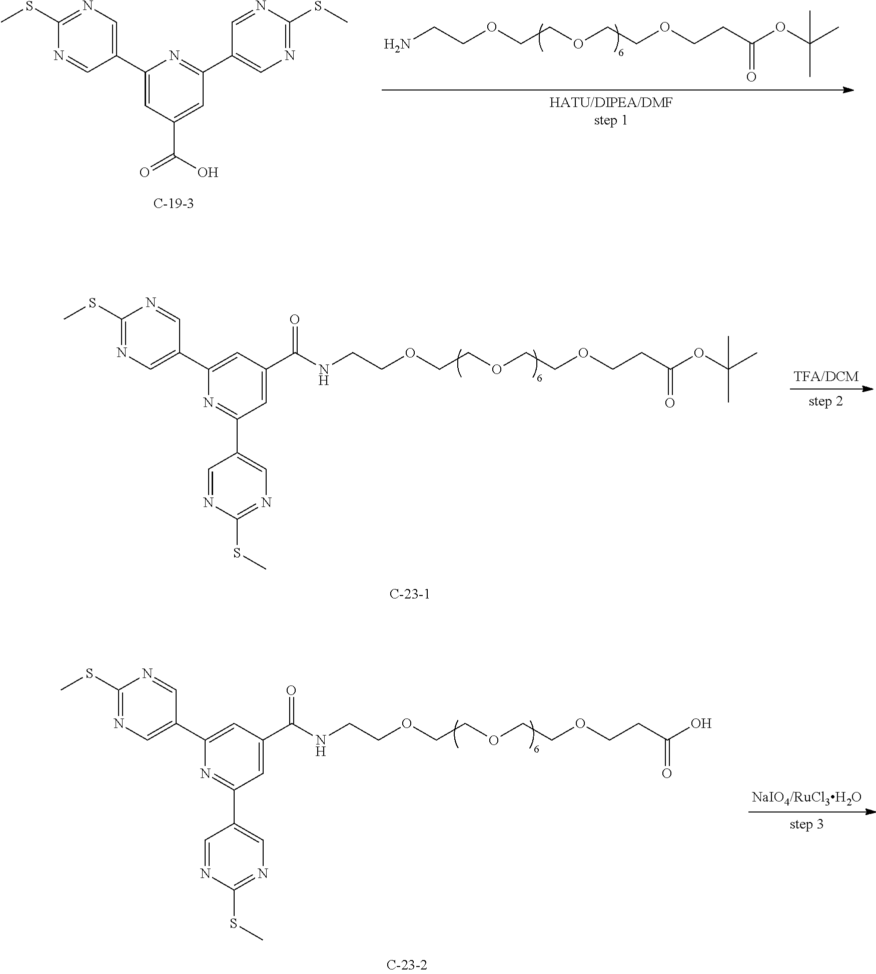

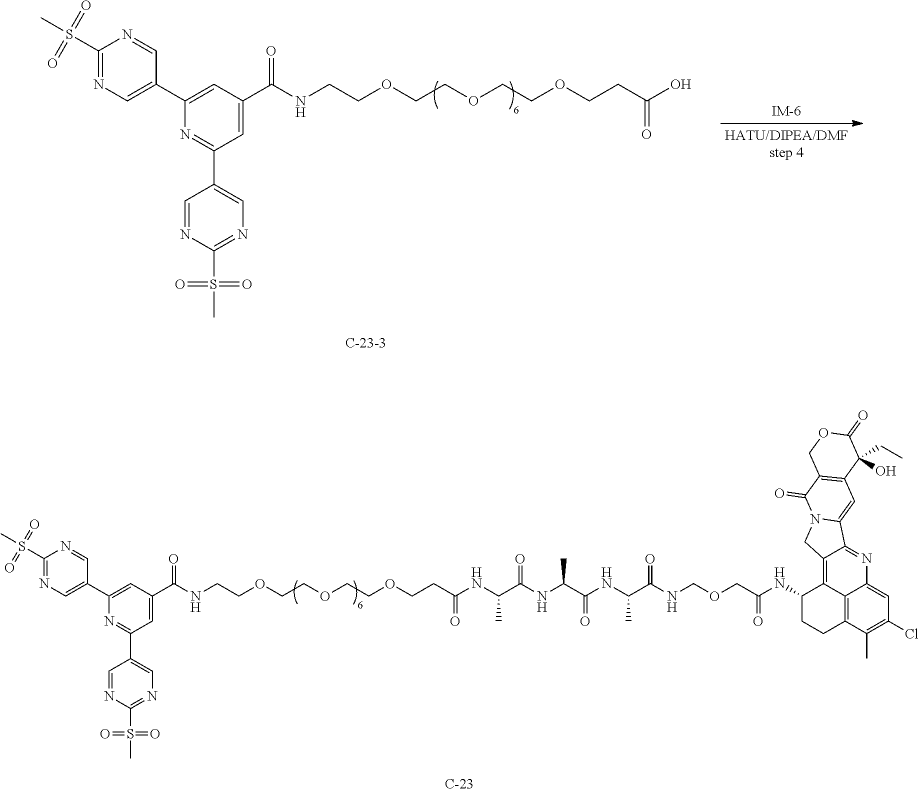

Step one: C-19-3 (1.50 g, 4.04 mmol) and tert-butyl 1-amino-3,6,9,12,15,18,21,24-octaoxaheptacosan-27-ate (2.01 g, 4.04 mmol) were added to DMF (20.0 mL), HOBt (1.64 g, 12.1 mmol), EDCI (2.32 g, 12.1 mmol) and DIEA (2.09 g, 16.2 mmol) were added sequentially, the temperature was raised to 60° C., and the reaction was stirred for 2 hours. The reaction was cooled to room temperature, water (10.0 mL) and ethyl acetate (20.0 mL) were added to the reaction solution to separate the layers. The aqueous phase was extracted twice with ethyl acetate (25.0 mL×2). The combined organic phases were dried over anhydrous sodium sulfate, filtered, and concentrated to obtain crude product C-23-1 (3.00 g), which was directly used in the next step.

Step two: C-23-1 (3.00 g, 3.53 mmol) was added to dichloromethane (10.0 mL), TFA (15.4 g, 134 mmol) was added, and the mixture was stirred at 25° C. for 12 hours. The reaction solution was directly concentrated to obtain a crude product, which was purified by preparative high-performance liquid chromatography and then freeze-dried to obtain C-23-2 (1.20 g).

Its preparation method was as follows:

Column: Welch Ultimate C18 (150 mm*25 mm*5 μm)

Mobile phase A: Acetonitrile; mobile phase B: Water (0.05% formic acid)

| Time | Mobile | Mobile | Flow rate |
|---|---|---|---|
| [min] | phase A [%] | phase B [%] | [mL/min] |
| 0.00 | 34 | 66 | 25 |
| 10.00 | 64 | 36 | 25 |

Step three: C-23-2 (500 mg, 0.63 mmol) was added to acetonitrile 10 mL and water (5 mL), ruthenium trichloride hydrate (13.0 mg, 0.063 mmol) and sodium periodate (1.35 g, 6.29 mmol) were added, and the system was reacted at 25° C. for 1 hour and then extracted with water (10 ml) and ethyl acetate (40 ml). The organic phase was concentrated to obtain a crude product, which was purified by column chromatography (MeOH/DCM=10.20%) and concentrated to obtain C-23-3 (350 mg).

Step four: IM-6 (20.0 mg, 0.025 mmol) and C-23-3 (21.5 mg, 0.025 mmol) were dissolved in DMF (1 mL), and HATU (19.0 mg, 0.050 mmol) and DIPEA (12.9 mg, 0.100 mmol) were added. After reacting at room temperature for 2 hours, the reaction solution was directly purified by preparative high-performance liquid chromatography and then freeze-dried to obtain C-23 (17.0 mg).

Its structural characterization data were as follows:

ESI-MS (m/z):1592.6 [M+H]⁺.

Its preparation method was as follows:

Column: Waters XBridge Prep C18OBD (5 m*19 mm*150 mm)

Mobile phase A: Acetonitrile; mobile phase B: Water (0.05% formic acid)

| Time | Mobile | Mobile | Flow rate |
|---|---|---|---|
| [min] | phase A [%] | phase B [%] | [mL/min] |
| 0.00 | 30 | 70 | 28 |
| 4.00 | 30 | 70 | 28 |
| 24.00 | 90 | 10 | 28 |

### Preparation of 4-((S)-2-(4-aminobutyl)-35-(4-((6-(2-(methylsulfonyl)pyrimidin-5-yl)hex-5-ynamido)methyl)-1H-1,2,3-triazol-1-yl)-4,8-dioxo-6,12,15,18,21,24,27,30,33-nonaoxa-3,9-diazapentatriacontanamido)benzyl ((1S,9R)-5-chloro-9-ethyl-1-(2-hydroxyacetamido)-4-methyl-10,13-dioxo-2,3,9,10,13,15-hexahydro-1H,12H-benzo[d]Pyrano[3′, 4′: 6,7]indolizino[1,2-b]quinolin-9-yl)carbonate (B-03)

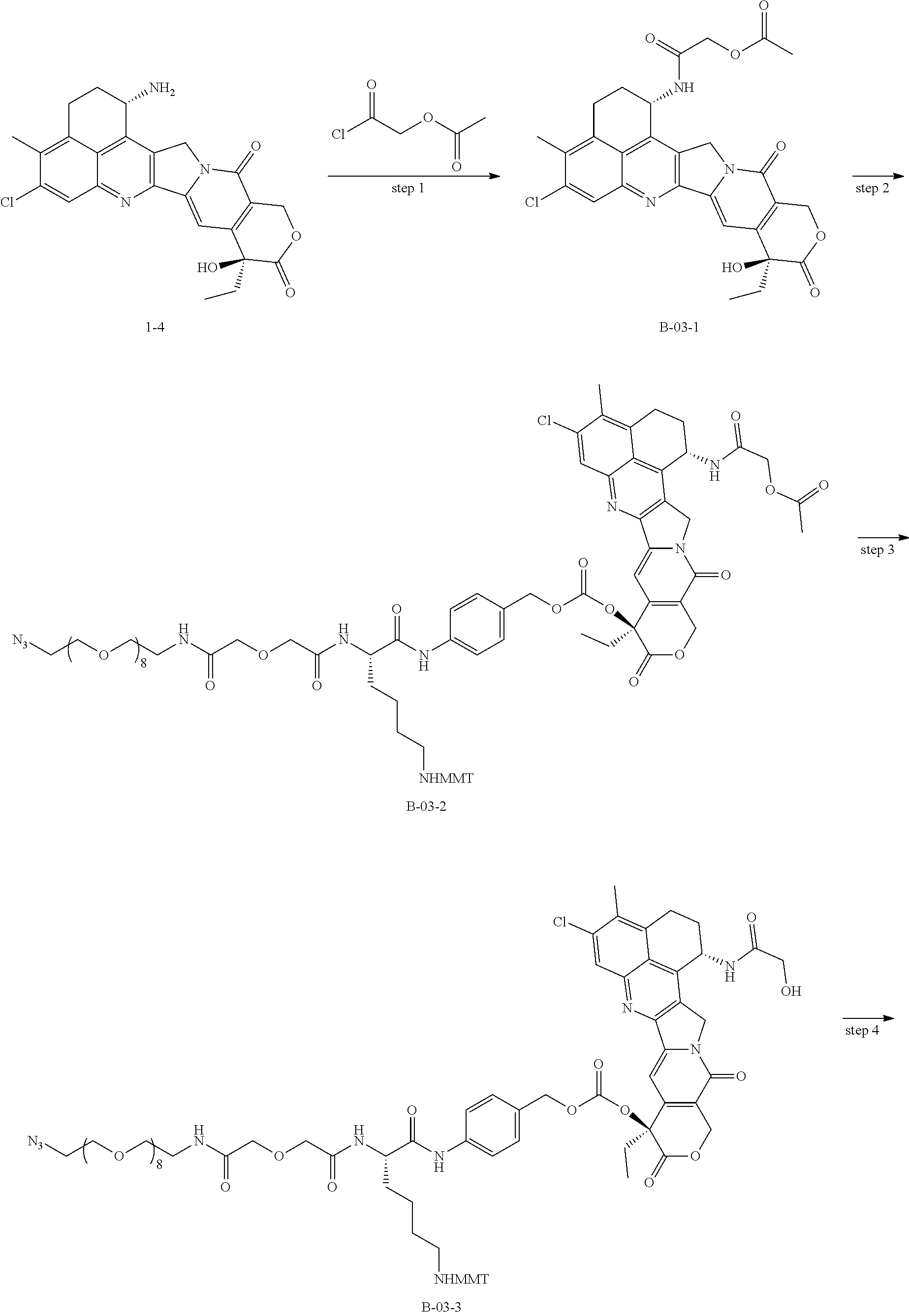

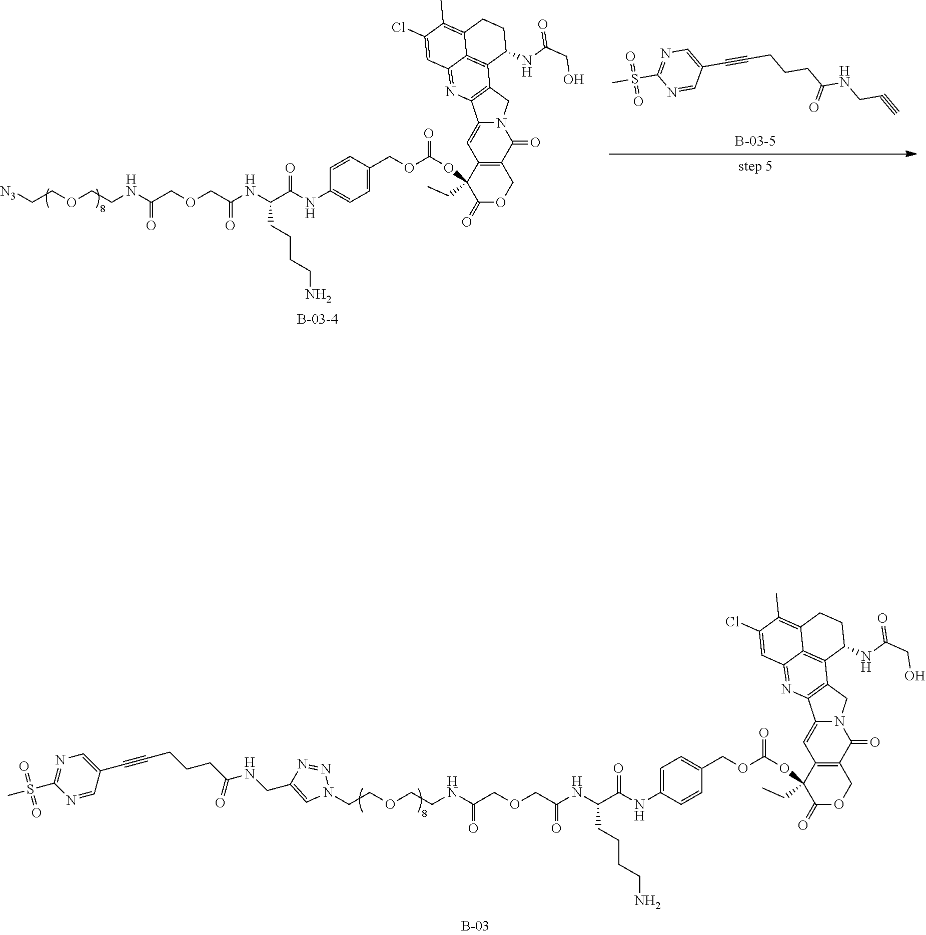

Step one: (1S,9S)-1-amino-5-chloro-9-ethyl-9-hydroxy-4-methyl-1,2,3,9,12,15-hexahydro-10H,13H-benzo[d]pyrano[3′, 4′: 6,7]indolizino[1,2-b]quinoline-10,13-dione (2 g, 3.65 mmol) was dissolved in DMF (50 mL), followed by dropwise addition of DIPEA (1.18 g, 9.12 mmol, 1.59 mL), and acetoxyacetyl chloride (548.12 mg, 4.01 mmol, 431.59 L) was added dropwise while cooling in an ice bath and stirring, and stirring was continued for 1 hour. The reaction solution was added to 0.1M dilute hydrochloric acid aqueous solution to precipitate the solid and filtered. The filter cake was dissolved in dichloromethane and methanol, dried over anhydrous sodium sulfate, filtered and concentrated to obtain a crude product, which was purified by a silica gel column (methanol/dichloromethane=0% to 5%) and concentrated again to obtain compound B-03-1 (1.7 g, 3.077 mmol).

Its structural characterization data were as follows:

ESI-MS (m/z): 552.2[M+1]+

Step two: Compound B-03-1 (500 mg, 0.905 mmol) and DMAP (885.33 mg, 7.25 mmol) were dissolved in dry dichloromethane (5 mL), cooled to 0° C. under nitrogen protection, and triphosgene (268.81 mg, 0.905 mmol) in dichloromethane (5 mL) was added dropwise, and stirred for 0.5 hours with the temperature maintained. (S)-2-(32-azido-5-oxo-3,9,12,15,18,21,24,27,30-nonaoxa-6-azadotriacontanamido)-N-(4-(hydroxymethyl)phenyl)-6-(((4-methoxyphenyl)diphenylmethyl) amino)hexanamide (1.44 g, 1.36 mmol) in dichloromethane was slowly dropped in, and the reaction was allowed to naturally return to room temperature and reacted for 4 hours. Water was added to quench the reaction, which was extracted with dichloromethane three times (100 mL×3). The organic phases were combined, washed with saturated brine, dried and concentrated. The crude product was purified by silica gel column (MeOH/DCM=0% to 5%) to obtain compound B-03-2 (498 mg, 0.304 mmol).

Its structural characterization data were as follows:

ESI-MS (m/z): 1352.8[M+1]⁺

Step three: Compound B-03-2 was dissolved in THE (3 mL) and MeOH (3 mL), and an aqueous solution (1 mL) of sodium carbonate (25.88 mg, 0.224 mmol) was added dropwise with stirring. After the dropwise addition, stirring was continued for 1 hour. Dilute hydrochloric acid was added dropwise into the reaction solution to neutralize the reaction, which was concentrated under reduced pressure and used directly in the next step.

Its structural characterization data were as follows:

ESI-MS (m/z): 1596.7[M+1]⁺

Step four: Compound B-03-3 (190 mg, 119.04 mol) was dissolved in dichloromethane (5 mL), trifluoroacetic acid (0.5 mL) was added, and the reaction continued for 1 hour. Saturated aqueous sodium bicarbonate solution was added dropwise to the reaction solution to neutralize and separate the layers, and the organic phase was concentrated to obtain a crude product. After purification by reversed-phase column chromatography (acetonitrile/1% formic acid aqueous solution=0% to 50%), compound B-03-4 (95 mg, 69.35 mol) was obtained by freeze-drying.

Its structural characterization data were as follows:

ESI-MS (m/z): 1323.6[M+1]⁺

Step five: Compound B-03-4 (90 mg, 0.066 mmol) and 6-(2-(methylsulfonyl)pyrimidin-5-yl)-N-(prop-2-yn-1-yl)hex-5-ynamide (24.07 mg, 0.079 mmol) were dissolved in DMSO (2 mL) and water (0.2 mL), copper bromide (9.42 mg, 0.066 mmol) was added, and stirring was continued for 2 hours. The reaction solution was directly filtered and concentrated to obtain a crude product, which was purified by preparative high-performance liquid chromatography and then freeze-dried to obtain compound B-03 (42.2 mg, 24.69 mol).

Its structural characterization data were as follows:

ESI-MS (m/z): 1628.7[M+1]⁺

Its separation and purification method was as follows:

Column: SunFire Prep C18 OBD 19 mmx 150 mmx 5.0 μm

Mobile phase A: acetonitrile; mobile phase B: water (0.05% formic acid)

| Time | Mobile | Mobile | Flow rate |
|---|---|---|---|
| [min] | phase A [%] | phase B [%] | [mL/min] |
| 0.00 | 10 | 90 | 28 |
| 2.00 | 10 | 90 | 28 |
| 18.00 | 90 | 10 | 28 |

### Example 2. Preparation of Antibodies

### 2.1. Construction and Expression of Recombinant Anti-EGFR and Anti-c-MET Bispecific Antibodies

### Fab-scFv-Fc(KIH) Bispecific Antibody

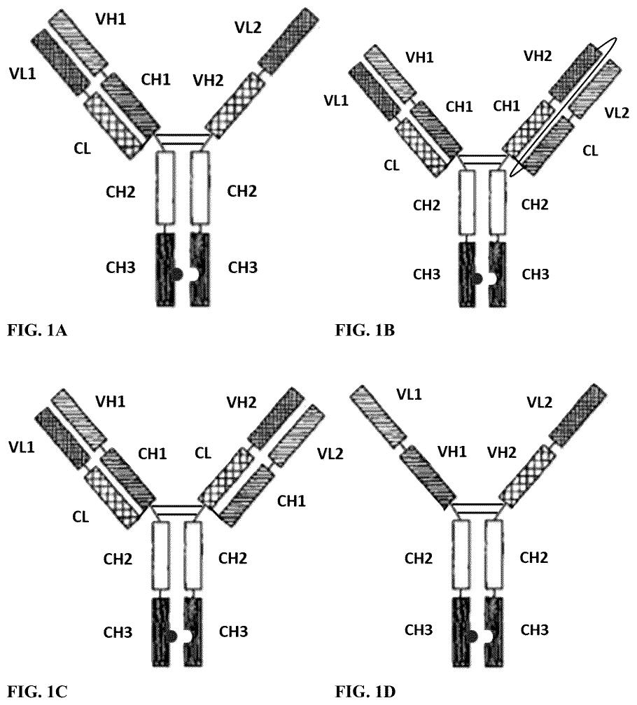

The bispecific antibody structure adopts the Fab-scFv-Fc (KIH) bispecific antibody structure, and a knob-in-hole (KIH) structure was used between the heavy chains to prevent mismatches. The structural diagram is shown in FIG. 1A. The amino acid sequence composition of anti-EGFR and anti-c-MET bispecific antibodies is shown in Table 1.

TABLE 1
Fab-scFv-Fc(KIH) bispecific antibody amino acid sequences

| BsAb | Type | LC | HC(knob) | scFv-Fc(hole) |
|---|---|---|---|---|
| 07B | Anti-c-MET | SEQ ID NO: 1 | SEQ ID NO: 2 | / |
| | Anti-EGFR | / | / | SEQ ID NO: 3 |
| 10B | Anti-EGFR | SEQ ID NO: 4 | SEQ ID NO: 5 | |
| | Anti-c-MET | / | / | SEQ ID NO: 6 |
| 38B | Anti-EGFR | SEQ ID NO: 4 | SEQ ID NO: 7 | / |
| | Anti-c-MET | / | / | SEQ ID NO: 8 |
| 49B | Anti-c-MET | SEQ ID NO: 1 | SEQ ID NO: 9 | / |
| | Anti-EGFR | / | / | SEQ ID NO: 10 |

The above amino acid sequences were sent to Genscript Biotechnology Co., Ltd. for codon optimization and DNA synthesis, and the light and heavy chains of the bispecific antibody were cloned into a pKL GS expression vector (containing two expression cassettes; designed by Sichuan Kelun-Biotech Biopharmaceutical Co., Ltd. and gene synthesis by GenScript). The promoter of the first expression cassette is the mouse CMV promoter and the polyA signal sequence is the sv40 polyA signal sequence. The promoter of the second expression cassette is the mouse CMV promoter and the polyA signal sequence is the thymidine kinase (TK) polyA signal sequence. The scFv-Fc of each bispecific antibody was separately cloned into a pKL5 Vector (containing 1 expression cassette; the promoter is the mouse CMV promoter and the polyA signal sequence is the CMV polyA signal sequence; designed by Sichuan Kelun-Biotech Biopharmaceutical Co., Ltd. and gene synthesis by GenScript or pKL8 expression vector (containing 1 expression cassette; the promoter is mouse CMV promoter and the polyA signal sequence is the CMV polyA signal sequence). For each plasmid pair of the plasmid expressing the light and heavy chains of a bispecific antibody and the plasmid expressing the bispecific antibody scFv-Fc as shown in Table 1, the plasmid pair was co-transfected into CHO-K1 cells (obtained from American Type Culture Collection (ATCC™), Manasas, VA) and the cells were cultured in a cell culture medium for expression of the bispecific antibodies. After allowing expression of the bispecific antibodies for a period of time, the bispecific antibodies were harvested from the culture medium and Protein A (MabSelect™ SuRe, GE) chromatography was used to capture the bispecific antibodies. The bispecific antibodies were then purified using cation (Eshmuno® CPX, Merck KGaA) chromatography to provide BsAb 07B, BsAb 10B, BsAb 38B, and BsB 49B.

### Fab-scFab-Fc (KIH) Bispecific Antibody

The bispecific antibody structure comprises the Fab-scFab-Fc (KIH) bispecific antibody structure and a KIH structure was used between the heavy chains to prevent mismatching. To increase stability, a pair of disulfide bonds were introduced into the scFab-Fc chain, that is, the Q at position 106 of scFab-Fc was mutated to C, and the G at position 304 was mutated to C. The structural diagram is shown in FIG. 1B. The amino acid sequence of the anti-EGFR and anti-c-MET bispecific antibodies are shown in Table 2.

TABLE 2
Fab-scFab-Fc (KIH) bispecific antibody amino acid sequences

| BsAb | Type | LC | HC(knob) | scFab-Fc(hole) |
|---|---|---|---|---|
| 41B | Anti-EGFR | SEQ ID NO: 4 | SEQ ID NO: 7 | |
| | Anti-c-MET | | / | SEQ ID NO: 11 |

The above amino acid sequences were sent to Genscript Biotechnology Co., Ltd. for codon optimization and DNA synthesis. The light and heavy chains of the bispecific antibody were cloned into a pKLGS expression vector (Sichuan Kelun-Biotech Biopharmaceutical Co., Ltd.), and the scFab-Fc of the bispecific antibody was cloned into a pKL5 expression vector (Sichuan Kelun-Biotech Biopharmaceutical Co., Ltd.). The plasmid pair of the plasmid expressing the light and heavy chains of the bispecific antibody and the bispecific antibody scFab-Fc were co-transfected into CHO-K1 cells (ATCC™) and the cells were cultured in cell culture medium for expression of the bispecific antibody 41B. After allowing expression of the bispecific antibodies for a period of time, the bispecific antibodies were harvested from the culture medium and captured by Protein A (MabSelect SuRe™ LX, GE) affinity Chromatography was used to capture the bispecific antibody. The bispecific antibodies were then purified using cation chromatography (Eshmuno® CPX, Merck KGaA) to provide BsAb 41B.

### CrossMab Bispecific Antibody

In this embodiment, the bispecific antibody structure comprises the CrossMab^{VH-VL} bispecific antibody structure, and the KIH structure was used between the heavy chains to prevent mismatches. The structural diagram is shown in FIG. 1C. The amino acid sequence composition of anti-EGFR and anti-c-MET bispecific antibodies is shown in Table 3.

TABLE 3
CrossMab^{VH-VL}
bispecific antibody amino acid sequences

| | | | | | VH2-CL- |
|---|---|---|---|---|---|
| BsAb | Type | LC | HC(knob) | VL2-CH1 | Fc(hole) |
| 55B | Anti-c-MET | SEQ ID | SEQ ID | | |
| | | NO: 1 | NO: 9 | | |
| | Anti-EGFR | | | SEQ ID | SEQ ID |
| | | | | NO: 12 | NO: 13 |

The above amino acid sequences were sent to Genscript Biotechnology Co., Ltd. for codon optimization and DNA synthesis. The light and heavy chains of BsAb 55B were cloned into a pKLGS expression vector (Sichuan Kelun-Biotech Biopharmaceutical Co., Ltd.), and the VL-CH1 and VH-CL-Fc of BsAb 55B were cloned into another pKLGS expression vector. The two pKLGS plasmids were co-transfected into CHO-K1 cells (ATCC™) and the cells were cultured in cell culture medium for expression of the BsAb 55B. After allowing expression of the bispecific antibodies for a period of time, the bispecific antibodies were harvested from the culture medium and Protein A (MabSelect SuRe™ LX, GE) affinity Chromatography was used to capture the bispecific antibody. The bispecific antibodies were then purified using cation chromatography (Eshmuno® CPX, Merck KGaA) to provide bispecific antibody 55B.

scFv-Fe(KIH) Bispecific Antibody

The bispecific antibody structure adopts the scFv-Fc (KIH) bispecific antibody structure, and a KIH structure was used between the heavy chains to prevent mismatches. The structural diagram is shown in FIG. 1D. The amino acid sequence composition of anti-EGFR and anti-c-MET bispecific antibodies is shown in Table 4.

TABLE 4
scFv-Fc(KIH) bispecific antibody amino acid sequences

| BsAb | Type | scFv-Fc(knob) | scFv-Fc(hole) |
|---|---|---|---|
| 56B | Anti-EGFR | SEQ ID NO: 14 | / |
| | Anti-c-MET | / | SEQ ID NO: 8 |

The above amino acid sequences were sent to Genscript Biotechnology Co., Ltd. for codon optimization and DNA synthesis. The scFv-Fc (knob) and scFv-Fc (hole) of the bispecific antibody were cloned into pKLGS expression vectors (Sichuan Kelun-Biotech Biopharmaceutical Co., Ltd.). The bispecific antibody-expressing plasmid was transfected into CHO-K1 cells (ATCC™) and the cells were cultured in cell culture medium for expression of BsAb 56B. After allowing expression of the bispecific antibodies for a period of time, the bispecific antibodies were harvested from the culture medium and Protein A (MabSelect SuRe™ LX, GE) affinity Chromatography was used to capture the bispecific antibody. The bispecific antibodies were then purified using cation (Eshmuno® CPX, Merck) chromatography to provide BsAb 56B.

2.2. Construction and Expression of Control Recombinant Anti-EGFR Monoclonal Antibodies and Anti-c-MET Monoclonal Antibodies, and hIgG1 Antibodies.

Anti-EGFR monoclonal antibody Zalutumumab is a parental monoclonal antibody of the bispecific antibody provided by the present invention. The amino acid sequence of the variable region of Zalutumumab is the same as the anti-EGFR portion of the bispecific antibody provided by the present invention. The amino acid sequence of the variable region of Zalutumumab may be obtained from the IMGT database using INN number 8605. The heavy chain variable region was fused to a mutated human IgG1 heavy chain constant region (SEQ ID NO: 64), and the light chain variable region was fused to a human kappa light chain constant region (SEQ ID NO: 53). MRG003 is an ADC based on an anti-EGFR monoclonal antibody in clinical phase II and this ADC has shown good efficacy in patients with nasopharyngeal carcinoma and head and neck cancer. BA03 is the antibody of MRG003. The BA03 monoclonal antibody amino acid sequence was obtained from Chinese patent CN106999606B. The anti-c-MET monoclonal antibody Onartuzumab is a parental monoclonal antibody of the bispecific antibody provided by the present invention. The amino acid sequence of the variable region of Onartuzumab is the same as the anti-c-MET portion of the bispecific antibody provided by the present invention. The amino acid sequence of the variable region of Onartuzumab may be obtained from the IMGT database using INN number 9368. The heavy chain variable region was fused to a mutated human IgG1 heavy chain constant region (SEQ ID NO:64), and the light chain variable region was fused to a human kappa light chain constant region (SEQ ID NO:53). ABBV399 is an anti-c-MET monoclonal antibody ADC in Phase III clinical trials and has shown good efficacy in patients with non-small cell lung cancer. Telisotuzumab is the antibody of ABBV399. The amino acid sequence of Telisotuzumab may be obtained from the IMGT database using INN number 10366. hIgG1 is an anti-chicken lysozyme antibody. The variable region sequence comes from patent CA2309763A1. The heavy chain variable region was fused to a mutated human IgG1 heavy chain constant region (SEQ ID NO: 64), and the light chain variable region was fused to a human kappa light chain constant region (SEQ ID NO:53). To obtain control monoclonal antibodies of Zalutumumab, Onartuzumab and Telisotuzumab, expression plasmids were constructed that encoded the heavy and light chains of the antibodies. The expression plasmids were separately transfected into CHO-K1 cells and the transfected cells were cultivated in culture medium under suitable conditions for expression of the antibodies into the culture medium. After allowing expression of the control antibodies for a period of time, the control antibodies were harvested from the culture medium and Protein A (MabSelect SuRe LX, GE) affinity Chromatography was used to capture the control antibodies. The control antibodies were then purified using cation (Eshmuno® CPX, Merck) chromatography to provide control antibodies Zalutumumab, Onartuzumab, Telisotuzumab, and hIgG1.

2.3. Anti-EGFR c-MET Control Bispecific Antibody Expression.

Anti-EGFR/c-MET bispecific antibody Amivantamab is a marketed bispecific antibody approved for the treatment of non-small cell lung cancer. The amino acid sequence may be obtained from the World Health Organization (WHO) International Non-proprietary name (INN) database. The Amivantamab was assigned INN number 11030. The bispecific antibody amino acid sequence was sent to Genscript Biotechnology Co., Ltd. for expression bispecific antibody Amivantamab. AZD9592 is an anti-EGFR and c-MET dual-antibody ADC in clinical phase I. The source of the antibody sequence is patent application publication WO2023083846A1. An expression plasmid expressing the AZD9592 antibody was transfected into CHO-K1 cells and the transfected cells were cultivated in culture medium under suitable conditions for expression of the AZD9592 antibody into the culture medium. After allowing expression of the AZD9592 antibody for a period of time, the AZD9592 antibody was harvested from the culture medium and Protein A (MabSelect SuRe LX, GE) affinity chromatography was used to capture the AZD9592 antibody.

### Example 3. Conjugation of Compounds Containing Cell Bioactive Molecules and Linkers to Antibodies

3.1. Preparation of ADC Comprising Monospecific Antibodies (Teliotuzumab, BA03, and hIgG1) Coupled to VC-MMAE (Vedotin)

Solutions of Telisotuzumab (Teliso; ABT-700, see U.S. Pat. No. 8,329,173), BA03 (see CN103772504B), and hIgG1 (anti-chicken lysozyme) antibodies were diluted to 3 mg/mL with 20 mM PB, and the pH was adjusted to pH 7.3 with 1 M Na₂HPO₄. 10 mM TCEP-HCl (Tris(2-carboxyethyl)phosphine hydrochloride, pH 7.3) was then added to the antibody solutions, mixed, and the resulting solutions were incubated overnight in an ice bath at 4° C. to produce a solution of reduced antibody. Then a solution of MC-VC-MMAE (vedotin; Chengdu Dingdang Times Pharmaceutical Technology Co., Ltd.) dissolved in N, N-dimethylacetamide was added to each reduced antibody solution at a molar ratio of 6:1 of MC-VC-MMAE to antibody, and the resulting solutions were incubated at 4° C. After reacting for 4 hours, the antibody conjugate was purified by Toyopearl™ PPG-600M (Tosoh) hydrophobic chromatography, and then the buffer was replaced with a 20 mM histidine buffer solution of pH 6.0 using Amicon Ultra-Centrifugal Filters (Millipore) to obtain the antibody conjugates Telisotuzumab vedotin (see U.S. patent Ser. No. 10/603,389), MRG003 (BA03-vedotin; see WO2023083846), and hIgG1-vedotin. For each type of the obtained antibody conjugates, the DAR value measured by mass spectrometry was approximately 4.

### 3.2. Preparation of AZD9592

The pH of a solution of AZD9592 antibody was adjusted to 7.4 with 1M Na₂ HPO₄. Then 10 mM TCEP-HCl (Tris(2-carboxyethyl)phosphine hydrochloride, pH 7.4) was added to the solution, mixed, and the reduced antibody solution was incubated at room temperature for 3.5 hours. Then a 10% (v/v) solution of SG3932 (see WO2023083846A1, purchased from Shanghai Xiyao Pharmaceutical Technology Co., Ltd.) dissolved in DMSO was added to each reduced antibody solution at a molar ratio of 12:1 of SG3932 to antibody, and the resulting solution was incubated at room temperature for 3 hours. An NAP™ gel column (Cytiva) was used to replace the buffer with a 20 mM histidine buffer of pH 6.0 to obtain the antibody drug conjugate AZD9592, with a DAR value of approximately 6 as measured by mass spectrometry.

3.3. Preparation of ADCs Comprising BsAb 07B, BsAb 10B, BsAb 38B, BsAb 41B, BsAb 49B, and hIgG1-Conjugated to A-05

Solutions of BsAb 07B, BsAb 10B, BsAb 38B, BsAb 41B, BsAb 49B and hIgG1 were diluted with 20 mM phosphate buffer (PB)+0.1 M Ethylenediaminetetraacetic acid (EDTA) pH 7.6 and the pH was adjusted to 7.6 with 1 M Na₂ HPO₄. Then, a 10 mM TCEP-HCl solution (pH 7.6) was added to the BsAb solutions, mixed well, and incubated at room temperature for 2 hours to produce reduced BsAb solutions. For bispecific antibodies 07B, 10B, 38B, and 49B, a 10 mM solution of A-05 dissolved in dimethyl sulfoxide was added to each reduced BsAb solution at a molar ratio of 8:1 of A-05 to antibody. For bispecific antibody 41B and antibody hIgG1, a 10 mM solution of A-05 dissolved in dimethyl sulfoxide was added to the reduced BsAb solution at a molar ratio of 10:1 of A-05 to antibody. Both resulting solutions were then incubated at room temperature for 2 hours. The buffer was then replaced with a 20 mM histidine buffer solution at pH 6.0 using NAP™ gel columns (Cytiva) to obtain ADC 07B-A-05, ADC 10B-A-05, ADC 38B-A-05, ADC 41B-A-05, ADC 49B-A-05, and hIgG1-A-05. The DAR values of ADC 07B-A-05, ADC 10B-A-05, ADC 38B-A-05, and ADC 49B-A-05 as measured by mass spectrometry were approximately 6, and the DAR values of ADC 41B-A-05 and hIgG1-A-05 were approximately 8.

3.4. Preparation of ADCs Comprising BsAb 38B, BsAb 49B, BsAb 55B, BsAb 56B, Onartuzumab, Zalutumumab, and hIgG1-Conjugated to A-14

Solutions of BsAb 38B, BsAb 49B, BsAb 55B, BsAb 56B, Onartuzumab, Zalutumumab, and hIgG1 were diluted with 20 mM PB+0.1 M EDTA pH 7.6, and pH was adjusted to 7.6 with 1 M Na₂ HPO₄. 10 mM TCEP-HCl solution (pH 7.6) was then added, mixed well, and incubated at room temperature for 2 hours to produce reduced BsAb solutions. For BsAb 38B and BsAb 49B, a 10 mM solution of A-14 dissolved in dimethyl sulfoxide was added to each reduced BsAb solution at a molar ratio of 8:1 of A-14 to antibody. For BsAb 55B, Onartuzumab, Zalutumumab, and hIgG1, a 10 mM solution of A-14 dissolved in dimethyl sulfoxide was added to each reduced BsAb solution at a molar ratio of 10:1 of A-14 to antibody. For BsAb 56B, a 10 mM solution of A-14 dissolved in dimethyl sulfoxide was added to each reduced BsAb solution at a molar ratio of 6:1 of A-14 to antibody. All the resulting solutions were incubated at room temperature for 2 hours. The buffer for each was then replaced with a 20 mM histidine buffer solution at pH 6.0 using NAP™ gel columns (Cytiva) to obtain ADC 38B-A-14, ADC 49B-A-14, ADC 55B-A-14, ADC 56B-A-14, ADC Onartuzumab-A-14, ADC Zalutumumab-A-14, and ADC hIgG1-A-14. The DAR values of ADC 38B-A-14 and ADC 49B-A-14 as measured by mass spectrometry were approximately 6, and the DAR values of ADC 55B-A-14, ADC Onartuzumab-A-14, ADC Zalutumumab-A-14, and ADC hIgG1-A-14 were approximately 8. The DAR value of ADC 56B-A-14 was approximately 4.

3.5. Preparation of ADCs Comprising BsAb 49B or hIgG1 Conjugated to B-01 ADC

Solutions of BsAb 49B and antibody hIgG1 were diluted to 3 mg/mL with 20 mM PB+0.1 M EDTA pH 7.60, and the pH was adjusted to 7.6 using 1 M Na₂ HPO₄. For BsAb 49B, a 10 mM solution of B-01 dissolved in dimethyl sulfoxide (8 molar equivalent/antibody) was added to each reduced BsAb solution. For IgG1, a 10 mM solution of B-01 dissolved in dimethyl sulfoxide was added to the reduced antibody solution at a molar ratio of 10:1 of B-01 to antibody. The resulting solutions were incubated at room temperature for 2 hours. The buffer for each was then replaced with a 20 mM histidine buffer solution at pH 6.0 using NAP™ gel columns (Cytiva) to obtain ADC 49B-B-01 and ADC hIgG1-B-01. The DAR value of ADC 49B-B-01 measured by mass spectrometry was approximately 6, and the DAR value of ADC hIgG1-B-01 was approximately 8.

### 3.6. Hydrophilicity Properties of Bispecific Antibodies

The hydrophilicity of the antibodies was detected by using Agilent 1260 with TSKgel Butyl-NPR column. Column temperature: 30° C., detection wavelength: 280 nm, flow rate: 0.5 ml/min, mobile phase A: 1.5 mol/L (NH₄)₂SO₄, mobile phase B: 25 mmol/L Na₂HPO₄, pH=7.0, 25% IPA. A suitable amount of the test sample was diluted with diluent (0.75 mol/L (NH₄)₂SO₄) to make a solution of 1.0 mg/ml as the test sample solution. The control antibodies were Tagitanlimab as a hydrophilic control (disclosed in WO2017148424) and Sacituzumab as a hydrophobic control (disclosed in WO2019114666). (Both antibodies are produced by Kelun-Biotech.) Each of the control antibodies were diluted with the diluent to make a solution of 1.0 mg/ml as the system suitability solution. About 40 g of sample was injected and analyzed in gradient elution: 0-3 min, maintaining mobile phase A of 95% and mobile phase B of 5%; 3-40 min, mobile phase B from 5% to 100%; 40-45 min, maintaining mobile phase A of 95% and mobile phase B of 5%. The hydrophobicity value of the test sample was calculated according to the control sample with the formula: (retention time of the test sample−retention time of the hydrophilic control)/(retention time of the hydrophobic control−retention time of the hydrophilic control), and the smaller the retention time and hydrophobicity value, the better the hydrophilicity of the antibody. The results are shown in Table 5. The bispecific antibodies all have good hydrophilicity. Good hydrophilicity is beneficial for the production of antibodies, quality control or conjugation with small molecules, etc., which will enhance the in vivo drug efficacy and improve the pharmacokinetic properties and the medication safety.

TABLE 5
Hydrophilicity properties of bispecific antibodies

| | Retention time | Hydrophobicity |
|---|---|---|
| Samples | (min) | value |
| Tagitanlimab | 11.009 | 0.00 |
| (hydrophilic | | |
| control) | | |
| Sacituzumab | 19.001 | 1.00 |
| (hydrophobic | | |
| control) | | |
| 07B | 14.734 | 0.47 |
| 10B | 15.702 | 0.59 |
| 38B | 15.332 | 0.54 |
| 41B | 11.516 | 0.06 |
| 49B | 11.368 | 0.04 |
| 55B | 10.871 | −0.02 |
| 56B | 13.909 | 0.36 |

### 3.7. Detection of Hydrophilicity of Different ADCs

Hydrophobicity of the ADCs was determined using an Agilent 1260 and the analytical column TSKgel© Butyl-NPR. The column temperature was 30° C., the detection wavelength was 280 nm, and the flow rate was: 0.5 mL/min, mobile phase A: 1.5 mol/L (NH₄)₂SO₄; mobile phase B: 25 mmol/L Na₂HPO₄, pH=7.0, 25% isopropanol alcohol (IPA). For analysis, a test sample of ADC was diluted to 1 mg/mL solution using the diluent 0.75 mol/L (NH₄)₂SO₄ to provide a test solution. The hydrophilic control antibody was Tagitanlimab and the hydrophobic control antibody was Sacituzumab. Control antibodies were produced by Sichuan Kelun-Biotech Biopharmaceutical Co., Ltd. using the diluent to make a 1 mg/mL test solution as the system suitability solution. About 40 g was injected and eluted using a gradient elution: 0-3 minutes with the mobile phase A at 95% and mobile phase B at 5%; then 3-40 minutes with mobile phase B increasing from 5% to 100%; then 40-45 minutes at mobile phase A at 95% and mobile phase B at 5%. After testing, the hydrophobicity value of the test sample was calculated based on the control sample. The calculation formula was: (Retention time of test sample−Retention time of hydrophilic control)/(Retention time of hydrophobic control−Retention time of hydrophilic control). The smaller the retention time and hydrophobicity value, the higher the hydrophilicity of the antibody.

The results are shown in Table 6. Bispecific ADC 07B-A-05, ADC 07B-A-14, ADC 38B-A-14, ADC 49B-A-14, ADC 49B-B-01, ADC 49B-A-05, ADC 41B-A-05, ADC 55B-A-14, and ADC 56B-A-14 all displayed good hydrophilicity, i.e., reduced hydrophobicity compared to the Sacituzumab control. Good hydrophilicity is beneficial to production and quality control, while improving in vivo drug efficacy and drug metabolism. Reducing hydrophobicity also reduces immunogenicity.

TABLE 6
Bispecific antibody hydrophilicity
determination of various ADCs

| | Retention time | Hydrophobicity |
|---|---|---|
| Sample | (minutes) | value |
| Tagitanlimab | 11.01 | 0.00 |
| (hydrophilic | | |
| control) | | |
| Sacituzumab | 19.00 | 1.00 |
| (hydrophobic | | |
| control) | | |
| ADC 07B-A-05 | 17.50 | 0.81 |
| ADC 07B-A-14 | 18.11 | 0.89 |
| ADC 10B-A-05 | 20.21 | 1.15 |
| ADC 38B-A-14 | 18.10 | 0.89 |
| ADC 49B-A-14 | 13.98 | 0.37 |
| ADC 49B-B-01 | 14.65 | 0.46 |
| ADC 49B-A-05 | 16.23 | 0.65 |
| ADC 41B-A-05 | 16.50 | 0.69 |
| ADC 55B-A-14 | 13.98 | 0.37 |
| ADC 56B-A-14 | 17.23 | 0.78 |

### Example 4. Detection of ADC Activity

4.1. Cell Affinity Detection of Anti-Human EGFR c-MET Bispecific Antibodies and their Respective Conjugates

A flow cytometer (Beckman, model Cytoflex) was used to detect the interaction between anti-human EGFR/c-MET bispecific antibodies and their conjugates and human gastric cancer cells MKN45 (Nanjing Cobioer Biosciences) and human gastric cancer cells overexpressing human EGFR MKN45-EGFR (Kelun-Biotech), human lung squamous cell carcinoma EBC-1 (Nanjing Cobioer Biosciences), human lung squamous cell carcinoma EBC-1-EGFR overexpressing human EGFR (Kelun-Biotech), human non-small cell lung cancer cell HCC827 (ATCC™), and human epidermal squamous cell carcinoma cell A431 (Nanjing Cobioer Biosciences). The protocol was as follows.

The adherent cells were digested with Trypsin-EDTA solution (0.25%) (Shanghai BasalMedia) and the cell density adjusted to 4.0×10⁶ per mL. The cells were washed twice with 1% BSA and resuspended in 1% BSA solution. Then 50 μL cell suspension was added to each well of a 96-well plate (the number of cells was 2×10⁵ per well). The antibody and its conjugate were diluted with 1% BSA (final concentration 10 g/mL, 3-fold gradient dilution, a total of 11 concentration points). Then 50 μL of the diluted antibody and ADC were added to the wells of the 96-well plate and the plate was incubated at 4° C. for 60 minutes. The wells of the 96-well plate were then washed twice with 1% BSA, 50 μL of diluted secondary antibody was added to each well, and the 96-well plate was incubated at 4° C. for 30 minutes. Then the cells were washed twice with 1% BSA, and the cells were resuspended in in 200 μL of 1% BSA and subjected to flow cytometry detection.

Data processing: the Median PE value was exported and then imported into GraphPad Prism 6 software to calculate EC₅₀.

The tumor cell affinity results for the anti-EGFR/c-MET bispecific antibodies and their conjugates are shown in Table 7 and Table 8. HCC827 and A431 cells highly express EGFR, MKN45 and EBC-1 cells highly express c-MET, and MKN45-EGFR and EBC-1-EGFR cells simultaneously highly express EGFR and c-MET. Regardless of the expression level of EGFR and c-MET, the bispecific antibodies could effectively bind. The tumor cell affinities of the tested BsAb 07B, BsAb 10B, BsAb 38B, BsAb 41B, BsAb 49B and their corresponding ADCs conjugated to A-05 were equivalent and displayed consistent affinity.

TABLE 7
Results of the affinity determination between anti-human EGFR/c-MET
BsAb 07B, BsAb 10B, and BsAb 41B and their respective ADCs
for EGFR and c-MET expressing tumor cells

| | EC₅₀(nM) | | | |
|---|---|---|---|---|
| Compound | A431 | MKN45 | EBC-1 | MKN45-EGFR |
| BsAb 07B | 2.66 | / | 1.46 | 1.41 |
| ADC 07B-A-05 | 2.43 | / | 1.47 | 1.23 |
| BsAb 10B | 2.19 | 1.10 | 1.51 | 2.74 |
| ADC10B-A-05 | 1.62 | 2.14 | 1.91 | 1.71 |
| BsAb 41B | 1.34 | 1.77 | / | / |
| ADC41B-A-05 | 0.48 | 0.69 | / | / |

Note:
“/” means not tested.

TABLE 8
Results of the affinity determination between anti-human EGFR/
c-MET BsAb 38B and BsAb 49B and their respective ADCs for EGFR
and c-MET expressing tumor cells

| | EC₅₀ (nM) | | | | |
|---|---|---|---|---|---|
| Compound | HCC827 | MKN45 | EBC-1 | MKN45-EGFR | EBC-1-EGFR |
| BsAb 38B | 0.65 | 1.16 | 1.04 | 1.19 | 0.99 |
| ADC 38B-A-14 | 0.62 | 0.68 | 1.29 | 1.04 | 0.73 |
| BsAb 49B | 0.30 | 0.34 | 1.32 | 0.41 | 0.98 |
| ADC 49B-A-14 | 0.70 | 1.06 | 1.51 | 0.97 | 1.04 |
| ADC 49B-B-01 | 0.74 | 1.36 | 1.13 | 0.83 | 1.21 |
| ADC 49B-A-05 | 0.70 | 1.58 | 0.95 | 1.26 | 0.63 |

4.2. Detection of Endocytic Activity of Anti-Human EGFR c-MET Bispecific Antibodies and their Conjugates

A flow cytometer (Thermo Fisher, model Attune™ NxT) was used to detect the effects of anti-EGFR/c-MET bispecific antibodies and their conjugates on human gastric cancer cells MKN45 (Nanjing Cobioer Biosciences) and human gastric cancer cells overexpressing human EGFR (MKN45-EGFR) (Sichuan Kelun-Biotech Biopharmaceutical Co., Ltd.), human lung squamous cell carcinoma EBC-1 (Nanjing Cobioer Biosciences), human lung squamous cell carcinoma EBC-1-EGFR overexpressing human EGFR (Sichuan Kelun-Biotech Biopharmaceutical Co., Ltd.), human non-small cell lung cancer cell HCC827 (from the ATCC™) and endocytic activity of human epidermal squamous cell carcinoma cell A431 (Nanjing Cobioer Biosciences). The adherent cells were digested with Trypsin-EDTA solution (0.25%) (Shanghai BasalMedia), the cells were counted, and the cell density was adjusted to 1×10⁵ cells/mL with complete culture medium. Then 100 μL cell suspension was added to each well of a 96-well plate (the number of cells was 1×10⁴ cells per well) and the plates were incubated at 37° C., CO₂ for 24 hours. The culture medium was removed and 50 μL of fresh complete culture medium was added to each well; complete culture medium was used to gradually dilute the bispecific antibody and ADC, with the final concentrations being 0.52, 2.08, 8.33, 33.30, 100.00, 200.00, 333.33, 500 μg/mL (a total of 8 concentration points). PHrodo® reagent (Sichuan Kelun-Biotech Biopharmaceutical Co., Ltd.) in complete culture medium was reduced to 12 μg/mL (the final concentration of PHrodo® was 3 μg/mL). The serially diluted antibody to be tested and ADC were mixed with the diluted PHrodo® reagent at the ratio of 1:1 (30 μL:30 μL), and the resultant mixture was incubated at room temperature for 30 minutes in the dark. 50 μL of the mixture was then added to the wells of a 96-well plate and incubated at 37° C., 5% CO₂ for 24 hours. Afterwards, the culture medium was removed, the wells were washed once with sterile PBS, and then 100 μL Trypsin-EDTA (0.25%) was added to each well to release the cells from the wells. Next, 100 μL complete culture medium was added to neutralize the trypsin and the cells were dispersed by pipetting and tested via FACS.

Data processing: The Median YL1-H value was exported then imported into GraphPad Prism 6 software to calculate EC₅₀. The results are shown in Table 9 and Table 10. The bispecific antibody displayed good endocytic activity on tumor cells and the endocytic activity of the ADC was consistent with that of the corresponding antibody.

TABLE 9
Endocytic activity assay results for the anti-human EGFR/c-MET
BsAb 07B and BsAb 10B and their respective ADCs

| | EC₅₀(nM) | |
|---|---|---|
| Compound | EBC-1 | MKN45-EGFR |
| BsAb 07B | 0.42 | 0.59 |
| ADC 07B-A-05 | 0.67 | 1.01 |
| BsAb 10B | 0.28 | 1.14 |
| ADC 10B-A-05 | 0.79 | 0.76 |

TABLE 10
Endocytic activity assay results for the anti-human EGFR/c-MET
BsAb 38B and BsAb 49B and their respective ADCs

| | EC₅₀(nM) | | | | |
|---|---|---|---|---|---|
| Compound | HCC827 | MKN45 | EBC-1 | MKN45-EGFR | EBC-1-EGFR |
| BsAb 38B | 0.53 | 1.04 | 0.49 | 1.05 | 0.29 |
| ADC 38B-A-14 | 0.67 | 1.20 | 0.57 | 0.34 | 0.27 |
| BsAb 49B | 0.63 | 2.06 | 0.19 | 0.96 | 0.27 |
| ADC 49B-A-14 | 0.53 | 1.14 | 0.72 | 0.51 | 0.21 |
| ADC 49B-B-01 | 0.49 | 1.13 | 0.29 | 1.27 | 0.24 |
| ADC 49B-A-05 | 0.47 | 1.22 | 0.3 | 1.11 | 0.29 |

4.3. Anti-Human EGFR c-MET Bispecific ADC In Vitro Cell Killing Assay

Trypsin-EDTA (0.25%) (Shanghai BasalMedia) solution was used to digest adherent human gastric cancer cells MKN45 (Nanjing Cobioer Biosciences), human gastric cancer cells overexpressing human EGFR MKN45-EGFR (Kelon Botai), and human lung squamous cells. Cancer cells EBC-1 (Nanjing Cobioer Biosciences), human lung squamous cell carcinoma EBC-1-EGFR overexpressing human EGFR (Kelen Botai) and human non-small cell lung cancer cells HCC827 (from the ATCC™). The cells were counted and the cell density was adjusted to 5×10⁴ cells/mL with complete medium, and 100 μl cell suspension was added to each well of the 96-well plate (the number of each type of cells was 5,000/well), the 96-well plate was placed in a 37° C., CO₂ constant temperature incubator for 24 h. Complete culture medium was used to dilute the ADC to be tested, starting with a final concentration of 800 nM, and diluted 3 times, with a total of 11 concentration points; 100 μl of the diluted ADC was added to a 96-well plate, and the plate was incubated at 37° C., 5% CO₂ for 5 days. 20 μl CCK8 reagent was added to each well for MKN45, MKN45-EGFR, EBC-1 and EBC-1-EGFR cells and incubated at 37° C. for 2 to 3 hours, a microplate reader was used to detect the OD_{450 nm} signal value, which was imported into GraphPad Prism 6 software to calculate IC₅₀. For HCC827 cells, 20 μl CCL reagent was added to each well, the plate was shaken at 600 rpm for 5 minutes, and a microplate reader was used to detect the luminescence signal value, which was imported into GraphPad Prism 6 software to calculate IC₅₀. The results are shown in Table 11. The tested dual-antibody ADCs had comparable killing activities on various cells. The killing activity of the bispecific antibody conjugate was significantly stronger than that of the hIgG1 antibody conjugate, indicating that the killing effect was mediated by the target.

TABLE 11
In vitro cell killing results of anti-human EGFR/c-MET
ADCs comprising BsAb 07B, BsAb 10B, BsAb 38B, and
BsAb 49B conjugated to A-05, A-14, or B-01

| | IC₅₀(nM) | | | | |
|---|---|---|---|---|---|
| Compound | HCC827 | MKN45 | EBC-1 | MKN45-EGFR | EBC-1-EGFR |
| ADC 07B-A-05 | / | 0.41 | 0.48 | 0.09 | / |
| ADC 10B-A-05 | / | 0.33 | 0.59 | 0.08 | / |
| ADC 38B-A-05 | / | 0.41 | 0.73 | 0.07 | / |
| ADC 41B-A-05 | / | 0.42 | 0.44 | 0.07 | / |
| ADC 49B-A-05 | 0.63 | 0.68 | 1.08 | 0.16 | 0.39 |
| ADC hIgG1-A-05 | 1675.00 | 418.30 | 501.20 | 261.40 | 267.60 |
| ADC 38B-A-14 | 0.08 | 0.30 | 1.03 | 0.10 | 0.38 |
| ADC 49B-A-14 | 0.21 | 0.57 | 2.93 | 0.14 | 0.29 |
| ADC 55B-A-14 | 0.14 | 0.40 | 0.42 | 0.10 | / |
| ADC 56B-A-14 | 0.30 | 1.17 | 2.95 | 0.27 | / |
| ADC hIgG1-A-14 | 126.90 | 48.73 | 132.40 | 38.43 | 61.64 |
| ADC 49B-B-01 | 3.43 | 0.78 | 0.66 | 0.17 | 0.37 |
| ADC hIgG1-B-01 | 6.15 | 0.85 | 0.91 | 0.56 | 0.91 |

Note:
“/” means not tested.

### 4.4. In Vivo Efficacy Testing of Different Antibody Conjugates MKN45 Model

Human gastric cancer cells MKN45 (purchased from Nanjing Cobioer Biosciences Co., Ltd.) were cultured as a monolayer in vitro in RPMI1640 medium containing 10% fetal calf serum at 37° C. in 5% CO₂. Cells were digested with trypsin-EDTA and passaged 2-3 times/week. When the cells were in the exponential growth phase, the culture medium was removed for mycoplasma detection and the cells were collected. Each mouse was subcutaneously inoculated into the right shoulder blade with 2.4×10⁶ MKN45 cells suspended in 0.1 mL PBS. When the average tumor volume had grown to about 100 to 200 mm³, mice with irregular, small, or large tumors were eliminated and the remaining mice were randomly divided into groups according to tumor volume and animal weight, with 6 mice in each group. The ADCs 07B-A-05, 10B-A-05, hIgG1-A-05, and 41B-A-05 were administered by tail vein injection (i.v.), once on the day of grouping. Dose settings: 07B-A-05 was administered at 3 mg/kg; 10B-A-05 was administered at 3 mg/kg and 10 mg/kg for different groups of mice, respectively; hIgG1-A-05 and 41B-A-05 were administered such that the molar dose of payload of each of hIgG1-A-05 and 41B-A-05 was the same as that of 07B-A-05. After grouping, the tumors were measured twice a week with a vernier caliper, and the tumor volume was calculated according to the following calculation formula: V=0.5a×b², wherein a and b represent the long and short diameters of a tumor, respectively; the death of animals was observed and recorded every day. Percent tumor growth inhibition (TGI) was calculated as follows: TGI (%) (tumor volume)=[1−(T_{Vt}−T_{V0})/(C_{Vt}−C_{V0})]×100%; when the tumor regresses, TGI (%)(tumor Volume)=100%−(T_{Vt}−T_{V0})/T_{V0}×100%. T_{V0} is the average tumor volume of the test drug group during group administration; T_{Vt} is the average tumor volume of the test drug group on days t after administration. C_{V0} is the average tumor volume of the vehicle group during group administration; C_{Vt} is the average tumor volume in the vehicle group on administration day t.

The results showed that ADCs 07B-A-05, 10B-A-05, and 41B-A-05 all demonstrated significant efficacy. The specific results are shown in Table 12 and FIG. 2.

TABLE 12
Analysis of the efficacy of different ADCs on
MKN45 cell tumor-bearing mouse models

| | Dosage | V_{P0}(mm³, | V_{P29}(mm³ | TGI |
|---|---|---|---|---|
| Compound | (mg/kg) | mean ± SEM) | mean ± SEM) | (%) |
| Saline | / | 158.42 ± 12.03 | 2083.19 ± 294.96 | / |
| ADC hIgG1-A-05 | 2.7 | 158.91 ± 12.06 | 1806.14 ± 275.55 | 14.42 |
| ADC 07B-A-05 | 3 | 158.75 ± 10.69 | 1169.46 ± 121.84 | 47.49 |
| ADC 10B-A-05 | 3 | 157.96 ± 11.96 | 1489.51 ± 173.46 | 30.82 |
| ADC 10B-A-05 | 10 | 155.97 ± 9.46 | 891.94 ± 101.12 | 61.76 |
| ADC 41B-A-05 | 2.9 | 159.33 ± 9.35 | 392.77 ± 42.89 | 87.72 |

### 4.5. In Vivo Efficacy Testing of Different Antibody Conjugated Drugs MKN45-EGFR Model

Human gastric cancer cells MKN45-EGFR were cultured in vitro as a monolayer culture in DMEM culture containing 10% fetal bovine serum and 3 g/mL Puromycin at 37° C. and 5% CO₂. Cells were digested with trypsin-EDTA and passaged 2-3 times/week. When the cells were in the exponential growth phase, the culture medium was removed for mycoplasma detection and the cells were collected. Each mouse was subcutaneously inoculated into the right shoulder blade with 2.5×10⁶ MKN45 cells suspended in 0.1 mL PBS. When the average tumor volume had grown to about 100 to 200 mm³, mice with irregular, small, or large tumors were eliminated and the remaining mice were randomly divided into groups according to tumor volume and animal weight, with 5 mice in each group. The bispecific ADCs 38B-A-14, ADC 38B-A-05, and ADC 49B-A-14, the monospecific ADCs hIgG1-A-14 (hIgG conjugated to A-14), Zalutumumab-A-14 (Zalutumumab conjugated to A-14), and Onartuzumab-A-14 (Onartuzumab conjugated to A-14) were administered by tail vein injection (i.v.), once on the day of grouping. Dose settings: 38B-A-14, 38B-A-05, 49B-A-14 were administered at 3 mg/kg, respectively; hIgG1-A-14, Onartuzumab-A-14 and Zalutumumab-A-14 were administered such that the molar dose of payload of each of hIgG1-A-14, Onartuzumab-A-14 and Zalutumumab-A-14 was the same as that of 38B-A-14. The ADCs were only administered once when grouped; Amivantamab (bispecific antibody anti-EGFR/c-MET marketed under the tradename RYBREVANT®, Janssen Pharmaceuticals, Inc.) was used as a comparator and was administered twice a week after grouping, for a total of 5 times with a dose of 10 mg/kg. After grouping, the tumors were measured twice a week with a vernier caliper, and the tumor volume was calculated according to the following calculation formula: V=0.5a×b², wherein a and b represent the long and short diameters of a tumor, respectively; the death of animals was observed and recorded every day. Percent TGI was calculated as follows: TGI (%) (tumor volume)=[1−(T_{Vt}−T_{V0})/(C_{Vt}−C_{V0})]×100%; when the tumor regresses, TGI (%)(tumor Volume)=100%−(T_{Vt}−T_{V0})/T_{V0}×100%. T_{V0} is the average tumor volume of the tested drug group during group administration; T_{Vt} is the average tumor volume of the tested drug group on days t after administration. C_{V0} is the average tumor volume of the vehicle group during group administration; C_{Vt} is the average tumor volume in the vehicle group on administration day t.

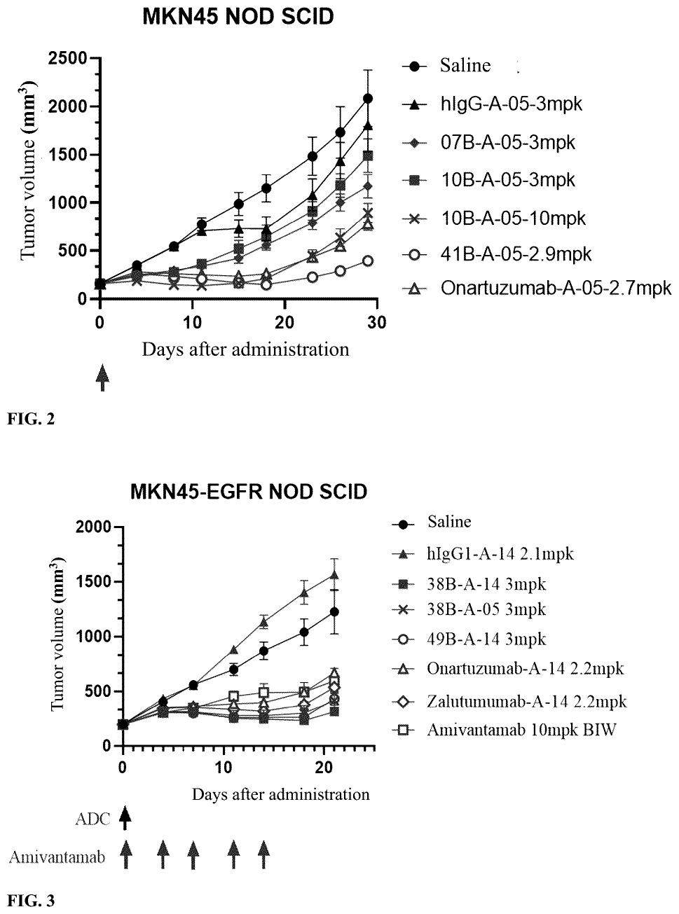

The results showed that the bispecific ADCs 38B-A-05, 38B-A-14, and 49B-A-14 were more effective than the isotoxin parent monoclonal antibody conjugate drugs ADC Onartuzumab-A-14 and ADC Zalutumumab-A-14, and bispecific antibody Amivantamab. The specific results are shown in Table 13 and FIG. 3.

TABLE 13
Analysis of the efficacy of different ADCs on
MKN45-EGFR cell tumor-bearing mouse models

| | Dosage | V_{P0}(mm³, | V_{P21}(mm³, | TGI |
|---|---|---|---|---|
| Compound | (mg/kg) | mean ± SEM) | mean ± SEM) | (%) |
| Saline | / | 198.32 ± 11.37 | 1226.14 ± 199.67 | / |
| ADC hIgG1-A-14 | 2.1 | 198.24 ± 11.58 | 1564.6 ± 147.6 | −32.94 |
| ADC 38B-A-14 | 3 | 198.49 ± 10.79 | 313.39 ± 30.29 | 88.82 |
| ADC 38B-A-05 | 3 | 196.97 ± 12.19 | 416.45 ± 85.67 | 78.65 |
| ADC 49B-A-14 | 3 | 196.72 ± 10.35 | 431.89 ± 59.74 | 77.12 |
| ADC | 2.2 | 197.26 ± 11.56 | 668.78 ± 17.08 | 54.12 |
| Onartuzumab-A-14 | | | | |
| ADC | 2.2 | 197.27 ± 10.36 | 536.64 ± 43.19 | 66.98 |
| Zalutumumab-A-14 | | | | |
| Amivantamab | 10 | 196.45 ± 10.84 | 595.29 ± 113.66 | 61.20 |

### 4.6. In Vivo Efficacy Testing of Different Antibody Conjugated Drugs MKN45 Model

Human gastric cancer cells MKN45 (purchased from Nanjing Cobioer Biosciences Co., Ltd.) were cultured in vitro as a monolayer in DMEM medium containing 10% fetal bovine serum at 37° C. and 5% CO₂. Cells were digested with trypsin-EDTA and passaged 2-3 times/week. When the cells were in the exponential growth phase, the culture medium was removed for mycoplasma detection and the cells were collected. Each mouse was subcutaneously inoculated into the right shoulder blade with 2.5×10⁶ MKN45 cells suspended in 0.1 mL PBS. When the average tumor volume had grown to about 100 to 200 mm³, mice with irregular, small, or large tumors were eliminated and the remaining mice were randomly divided into groups according to tumor volume and animal weight, with 6 mice in each group. The bispecific ADCs 38B-A-14 and 49B-A-14, the monospecific ADCs MRG003, Teliso-v, hIgG1-A-14, hIgG1-vedotin, Zalutumumab-A-14, and Onartuzumab-A-14 were administered via tail vein injection (i.v.), once on the day of grouping. Dose settings: 38B-A-14, 49B-A-14 were administered at 3 mg/kg, respectively; hIgG1-A-14, Onartuzumab-A-14 and Zalutumumab-A-14 were administered such that the molar dose of payload of each of hIgG1-A-14, Onartuzumab-A-14 and Zalutumumab-A-14 was the same as that of 38B-A-14; hIgG1-vedotin, Teliso-v, and MRG003 were administered such that the molar dose of protein of each of hIgG1-vedotin, Teliso-v, and MRG003 was the same as 38B-A-14. After grouping, the tumors were measured twice a week with a vernier caliper, and the tumor volume was calculated according to the following calculation formula: V=0.5a×b², wherein a and b represent the long and short diameters of a tumor, respectively; the death of animals was observed and recorded every day. Percent TGI was calculated as follows: TGI (%) (tumor volume)=[1−(T_{Vt}−T_{V0})/(C_{Vt}−C_{V0})]×100%; when the tumor regresses, TGI (%)(tumor Volume)=100%−(T_{Vt}−T_{V0})/T_{V0}×100%. T_{V0} is the average tumor volume of the tested drug group during group administration; T_{Vt} is the average tumor volume of the tested drug group on days t after administration. C_{V0} is the average tumor volume of the vehicle group during group administration; C_{Vt} is the average tumor volume in the vehicle group on day t.

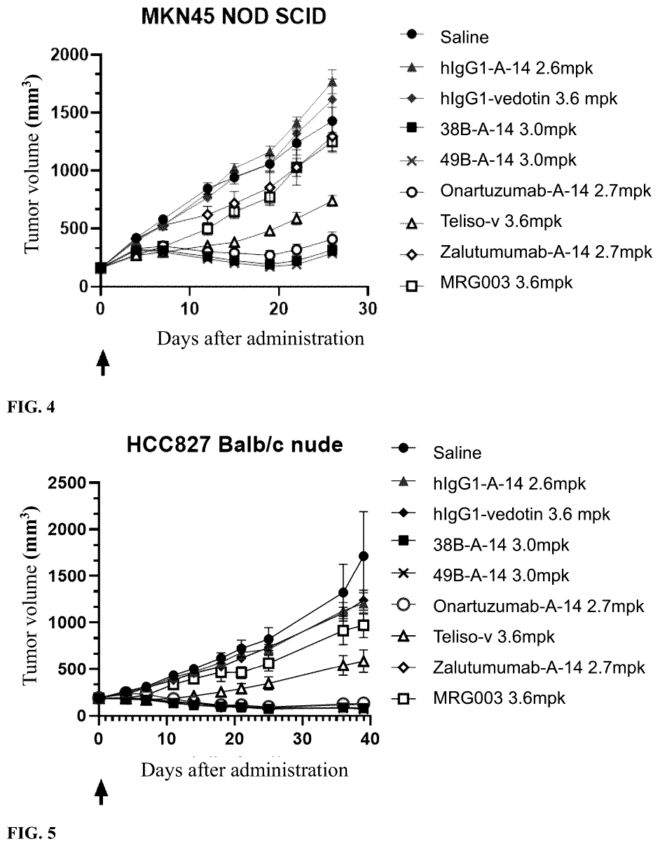

The results showed that the bispecific ADCs 38B-A-14 and 49B-A-14 were more effective than the clinical stage monospecific ADCs MRG003, Teliso-v, and the isotoxin parent monoclonal ADCs Zalutumumab-A-14 and Onartuzumab-A-14. The specific results are shown in Table 14 and FIG. 4.

TABLE 14
Analysis of the efficacy of different ADCs on
MKN45 cell tumor-bearing mouse models

| | Dosage | V_{P0}(mm³, | V_{P26}(mm³, | TGI |
|---|---|---|---|---|
| Drug | (mg/kg) | mean ± SEM) | mean ± SEM) | (%) |
| Saline | / | 161 ± 7.8 | 1428.63 ± 116.9 | / |
| ADC hIgG1-A-14 | 2.6 | 160.8 ± 9.12 | 1769.47 ± 103.38 | −26.9 |
| ADC hIgG1-vedotin | 3.6 | 160.98 ± 6.76 | 1614.88 ± 177.36 | −14.7 |
| ADC 38B-A-14 | 3 | 161.88 ± 8.3 | 316.04 ± 46.72 | 87.8 |
| ADC 49B-A-14 | 3 | 161.3 ± 6.38 | 287.15 ± 28.06 | 90.1 |
| ADC Onartuzumab-A-14 | 2.7 | 161.11 ± 6.73 | 409.25 ± 63.79 | 80.4 |
| ADC Teliso-v | 3.6 | 160.62 ± 7.24 | 740.15 ± 47.51 | 54.3 |
| ADC Zalutumumab-A-14 | 2.7 | 160.21 ± 7.82 | 1297.61 ± 138.67 | 10.3 |
| ADC MRG003 | 3.6 | 162.28 ± 8.74 | 1250.2 ± 79.98 | 14.2 |

### 4.7. In Vivo Efficacy Testing of Different Antibody Conjugated Drugs HCC827 Model

Human non-small cell lung cancer cells HCC827 (purchased from Nanjing Cobioer Biosciences Co., Ltd.) were cultured in monolayer in vitro in RPMI1640 medium containing 10% fetal calf serum at 37° C. and 5% CO₂. Cells were digested with trypsin-EDTA and passaged 2-3 times/week. When the cells were in the exponential growth phase, the culture medium was removed for mycoplasma detection and the cells were collected. Each mouse was subcutaneously inoculated into the right shoulder blade with 5×10⁶ HCC827 cells suspended in 0.1 mL PBS. When the average tumor volume had grown to about 100 to 200 mm³, mice with irregular, small, or large tumors were eliminated and the remaining mice were randomly divided into groups according to tumor volume and animal weight, with 6 mice in each group. The bispecific ADCs 38B-A-14 and 49B-A-14, the monospecific ADCs MRG003, Teliso-v, hIgG1-A-14, hIgG1-vedotin, Zalutumumab-A-14, and Onartuzumab-A-14 were administered via tail vein injection (i.v.), once on the day of grouping. Dose setting: 38B-A-14, 49B-A-14 were administered at 3 mg/kg, respectively; hIgG1-A-14, Onartuzumab-A-14 and Zalutumumab-A-14 was administered such that the molar dose of payload of each of hIgG1-A-14, Onartuzumab-A-14 and Zalutumumab-A-14 was the same as 38B-A-14; hIgG1-vedotin, Teliso-v, and MRG003 were administered such that the molar dose of protein of each of hIgG1-vedotin, Teliso-v, and MRG003 was the same as 38B-A-14. After grouping, the tumors were measured twice a week with a vernier caliper, and the tumor volume was calculated according to the following calculation formula: V=0.5a×b², wherein a and b represent the long and short diameters of a tumor, respectively; the death of animals was observed and recorded every day. Percent TGI was calculated as follows: TGI (%) (tumor volume)=[1−(T_{Vt}−T_{V0})/(C_{Vt}−C_{V0})]×100%; when the tumor regresses, TGI (%)(tumor Volume)=100%−(T_{Vt}−T_{V0})/T_{V0}×100%. T_{V0} is the average tumor volume of the tested drug group during group administration; T_{Vt} is the average tumor volume of the tested drug group on days t after administration. C_{V0} is the average tumor volume of the vehicle group during group administration; C_{Vt} is the average tumor volume in the vehicle group on administration day t.

The results showed that the bispecific ADCs 38B-A-14 and 49B-A-14 were more effective than clinical stage drugs MRG003, Teliso-v, and isotoxin parent monoclonal ADCs Zalutumumab-A-14 and Onartuzumab-A-14. The specific results are shown in Table 15 and FIG. 5.

TABLE 15
Analysis of the efficacy of different antibody
conjugated drugs on HCC827 cell tumor-bearing mouse model

| | Dosage | V_{P0}(mm³, | V_{P39}(mm³, | TGI |
|---|---|---|---|---|
| Drug | (mg/kg) | mean ± SEM) | mean ± SEM) | (%) |
| Saline | / | 190.29 ± 13.08 | 1716.47 ± 478.35 | / |
| ADC hIgG1-A-14 | 2.6 | 191.61 ± 13.74 | 1203.91 ± 116.19 | 33.7 |
| ADC hIgG1-vedotin | 3.6 | 194.11 ± 15.92 | 1237.92 ± 108.62 | 31.6 |
| ADC 38B-A-14 | 3 | 185.22 ± 13.07 | 84.83 ± 12.25 | 154.2 |
| ADC 49B-A-14 | 3 | 189.03 ± 19.94 | 74.7 ± 14.66 | 160.5 |
| ADC | 2.7 | 192.32 ± 22.06 | 135.02 ± 46.64 | 129.8 |
| Onartuzumab-A-14 | | | | |
| ADC Teliso-v | 3.6 | 189.05 ± 14.9 | 585.89 ± 120.65 | 74.0 |
| ADC | 2.7 | 187 ± 15.35 | 123.93 ± 41.3 | 133.7 |
| Zalutumumab-A-14 | | | | |
| ADC MRG003 | 3.6 | 188.27 ± 12.35 | 970.76 ± 131.53 | 48.7 |

### 4.8. In Vivo Efficacy Testing of Different Antibody Conjugated Drugs EBC-1-EGFR Model

Human non-small cell lung cancer cells EBC-1-EGFR were cultured in vitro in a monolayer in DMEM medium containing 10% fetal calf serum and 1 g/mL Puromycin at 37° C. and 5% CO₂. Cells were digested with trypsin-EDTA and passaged 2-3 times/week. When the cells were in the exponential growth phase, the culture medium was removed for mycoplasma detection and the cells were collected. Each mouse was subcutaneously inoculated into the right shoulder blade with 5×10⁶ EBC-1-EGFR cells suspended in 0.1 mL PBS. When the average tumor volume had grown to about 100 to 200 mm³, mice with irregular, small, or large tumors were eliminated and the remaining mice were randomly divided into groups according to tumor volume and animal weight, with 5 mice in each group. The bispecific ADC 49B-A-14 was administered via tail vein injection (i.v.) at 1 mg/kg and 3 mg/kg for different groups of mice, respectively; hIgG1-A-14 was administered such that the molar dose of payload of hIgG1-A-14 was the same as that of 49B-A-14 at the dose of 3 mg/kg; Onartuzumab-A-14 and Zalutumumab-A-14 were administered such that the molar dose of payload of each of Onartuzumab-A-14 and Zalutumumab-A-14 was the same as that of 49B-A-14 at the dose of 1 mg/kg; hIgG1-vedotin, Teliso-v, and MRG003 were administered such that the molar dose of protein of each of hIgG1-vedotin, Teliso-v, and MRG003 was the same as that of 49B-A-14 at the dose of 1 mg/kg. After grouping, the tumors were measured twice a week with a vernier caliper, and the tumor volume was calculated according to the following calculation formula: V=0.5a×b², wherein a and b represent the long and short diameters of a tumor, respectively; the death of animals was observed and recorded every day. Percent tumor growth inhibition (TGI) was calculated as follows: TGI (%) (tumor volume)=[1−(T_{Vt}−T_{V0})/(C_{Vt}−C_{V0})]×100%; when the tumor regresses, TGI (%)(tumor Volume)=100%−(T_{Vt}−T_{V0})/T_{V0}×100%. T_{V0} is the average tumor volume of the tested drug group during group administration; T_{Vt} is the average tumor volume of the tested drug group on days t after administration. C_{V0} is the average tumor volume of the vehicle group during group administration; C_{Vt} is the average tumor volume in the vehicle group on administration day t.

The results showed that the bispecific ADC 49B-A-14 completely regressed the tumor at 3 mg/kg and the TGI at the dose of 1 mg/kg was 69.89%. The efficacy was greater than that observed for monospecific ADCs MRG003, Teliso-v, and the isotoxin parent monoclonal ADCs Zalutumumab-A-14 and Onartuzumab-A-14. The specific results are shown in Table 16 and FIG. 6.

TABLE 16
Analysis of the efficacy of different ADCs on
EBC-1-EGFR cell tumor-bearing mouse model

| | Dosage | V_{P0}(mm³, | V_{P28}(mm³, | TGI |
|---|---|---|---|---|
| Drug | (mg/kg) | mean ± SEM) | mean ± SEM) | (%) |
| Saline | / | 147 ± 12.36 | 2121.27 ± 226.93 | / |
| ADC hIgG1-A-14 | 2.7 | 148.63 ± 13.54 | 1900.59 ± 210.2 | 11.26 |
| ADC hIgG1-vedotin | 1.2 | 147.29 ± 13.47 | 2124.9 ± 361 | −0.17 |
| ADC 49B-A-14 | 1 | 149.36 ± 12.02 | 743.8 ± 246.91 | 69.89 |
| ADC 49B-A-14 | 3 | 152.78 ± 15.79 | 0 ± 0 | 200.00 |
| ADC | 0.9 | 148.56 ± 18.36 | 1514.41 ± 341.97 | 30.82 |
| Onartuzumab-A-14 | | | | |
| ADC Teliso-v | 1.2 | 147.25 ± 12.8 | 981.94 ± 257.46 | 57.72 |
| ADC | 0.9 | 145.9 ± 11.34 | 1173.32 ± 164.67 | 47.96 |
| Zalutumumab-A-14 | | | | |
| ADC MRG003 | 1.2 | 148.6 ± 13.93 | 1890.29 ± 277.85 | 11.78 |

### 4.9 In Vivo Efficacy Testing of Different Antibody-Conjugated Drug HCC827 Models

Human non-small cell lung cancer cells HCC827 (purchased from Nanjing Cobioer Biosciences Co., Ltd.) were cultured in vitro in a monolayer in RPMI1640 medium with 10% fetal calf serum at 37° C. and 5% CO₂. Cells were digested with trypsin-EDTA and passaged 2-3 times/week. When the cells were in the exponential growth phase, the culture medium was removed for mycoplasma detection and the cells were collected. Each mouse was subcutaneously inoculated into the right shoulder blade with 5×10⁶ HCC827 cells suspended in 0.1 mL serum-free RPMI1640. When the average tumor volume had grown to about 100 to 200 mm³, mice with irregular, small, or large tumors were eliminated and the remaining mice were randomly divided into groups according to tumor volume and animal weight, with 5 mice in each group. The bispecific ADCs 49B-A-14 and AZD9592 were administered via tail vein injection (i.v.) at 1 mg/kg, once a week (two times in total). After grouping, the tumors were measured twice a week with a vernier caliper, and the tumor volume was calculated according to the following calculation formula: V=0.5a×b², wherein a and b represent the long and short diameters of a tumor, respectively; the death of animals was observed and recorded every day. Percent tumor growth inhibition (TGI) was calculated as follows: TGI (%) (tumor volume)=[1−(T_{Vt}−T_{V0})/(C_{Vt}−C_{V0})]×100%; when the tumor regresses, TGI (%)(tumor Volume)=100%−(T_{Vt}−T_{V0})/T_{V0}×100%. T_{V0} is the average tumor volume of the tested drug group during group administration; T_{Vt} is the average tumor volume of the tested drug group on days t after administration. C_{V0} is the average tumor volume of the vehicle group during group administration; C_{Vt} is the average tumor volume in the vehicle group on administration day t.

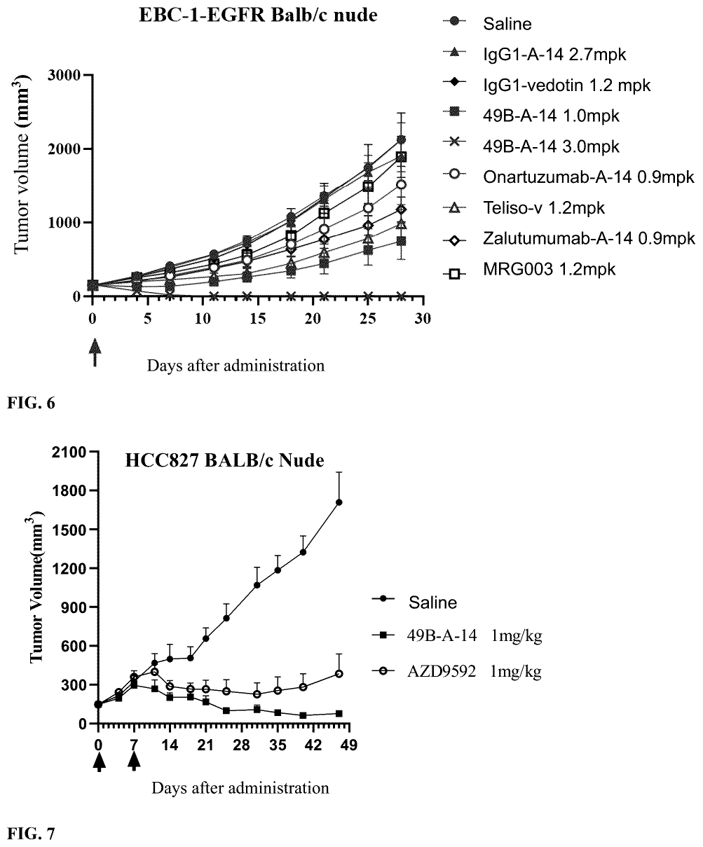

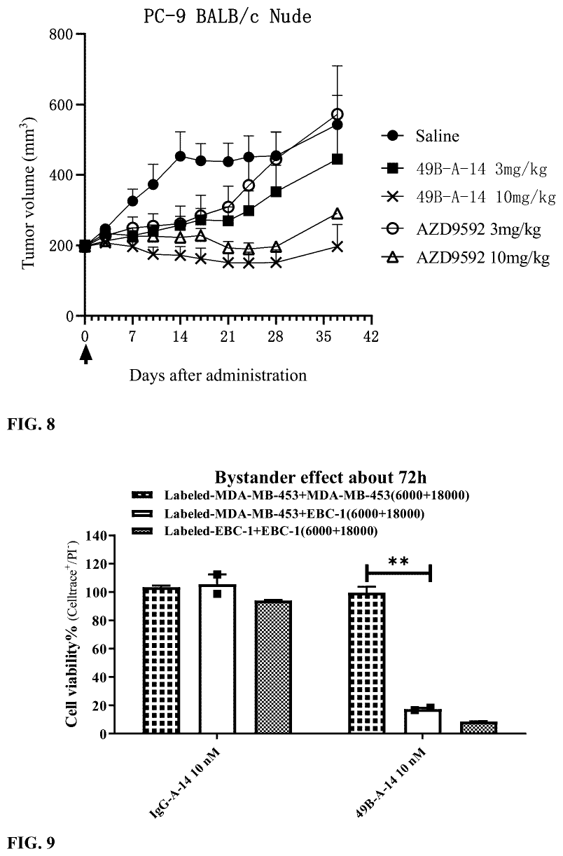

The results showed that the TGI of the bispecific antibody conjugate drug 49B-A-14 in the 1 mg/kg group was 147.69%, and the efficacy was significant. The TGI of AZD9592 in the 1 mg/kg group was 84.80%. 49B-A-14 was significantly better than AZD9592 (**, p<0.01). The specific results are shown in Table 17 and FIG. 7.

TABLE 17
Analysis of the efficacy of different ADCs on
HCC827 cell tumor-bearing mouse model

| | Dosage | V_{P0}(mm³, | V_{P47}(mm³, | TGI |
|---|---|---|---|---|
| Drug | (mg/kg) | mean ± SEM) | mean ± SEM) | (%) |
| Saline | / | 150.45 ± 11.24 | 1707.55 ± 232.81 | / |
| 49B-A-14 | 1 | 147.38 ± 9.93 | 77.1 ± 14.58 | 147.69 |
| AZD9592 | 1 | 148.09 ± 10.78 | 384.84 ± 151.86 | 84.80 |

### 4.10 In Vivo Efficacy Testing of Different Antibody Conjugated Drugs PC-9 Model

Human non-small cell lung cancer cells PC-9 (purchased from Nanjing Cobioer Biosciences Co., Ltd.) were cultured in vitro in a monolayer in RPMI1640 medium with 10% fetal calf serum at 37° C. and 5% CO₂. Cells were digested with trypsin-EDTA and passaged 2-3 times/week. When the cells were in the exponential growth phase, the culture medium was removed for mycoplasma detection and the cells were collected. Each mouse was subcutaneously inoculated into the right scapula with 5×10⁶ PC-9 cells suspended in 0.1 mL serum-free RPMI1640 containing 50% Matrigel. When the average tumor volume had grown to about 100 to 200 mm³, mice with irregular, small, or large tumors were eliminated and the remaining mice were randomly divided into groups according to tumor volume and animal weight, with 5 mice in each group. The bispecific ADCs 49B-A-14 and AZD9592 were administered once on the day of grouping via tail vein injection (i.v.) at 3 mg/kg and 10 mg/kg doses for each. After grouping, the tumors were measured twice a week with a vernier caliper, and the tumor volume was calculated according to the following calculation formula: V=0.5a×b², wherein a and b represent the long and short diameters of a tumor, respectively; the death of animals was observed and recorded every day. Percent tumor growth inhibition (TGI) was calculated as follows: TGI (%) (tumor volume)=[1−(T_{Vt}−T_{V0})/(C_{Vt}−C_{V0})]×100%; when the tumor regresses, TGI (%)(tumor Volume)=100%−(T_{Vt}−T_{V0})/T_{V0}×100%. T_{V0} is the average tumor volume of the tested drug group during group administration; T_{Vt} is the average tumor volume of the tested drug group on days t after administration. C_{V0} is the average tumor volume of the vehicle group during group; C_{Vt} is the average tumor volume in the vehicle group on administration day t.

The results showed that the TGI of the bispecific antibody conjugate drug 49B-A-14 at doses of 3 mg/kg and 10 mg/kg was 28.81% and 100.03% respectively, and the TGI of AZD9592 at doses of 3 mg/kg and 10 mg/kg was −8.86% and 72.83% respectively. 49B-A-14 was significantly better than AZD9592 at 10 mg/kg (*, p<0.05). The specific results are shown in Table 18 and FIG. 8.

TABLE 18
Analysis of the efficacy of different ADCs on PC-9
cell tumor-bearing mouse model

| | Dosage | V_{P0}(mm³, | V_{P37}(mm³, | TGI |
|---|---|---|---|---|
| Drug | (mg/kg) | mean ± SEM) | mean ± SEM) | (%) |
| Saline | / | 197.61 ± 12.49 | 543.02 ± 83.04 | |
| 49B-A-14 | 3 | 199.22 ± 14.08 | 445.11 ± 91.60 | 28.81 |
| 49B-A-14 | 10 | 196.47 ± 12.41 | 196.40 ± 62.50 | 100.03 |
| AZD9592 | 3 | 196.26 ± 18.65 | 572.26 ± 137.06 | −8.86 |
| AZD9592 | 10 | 197.09 ± 11.46 | 290.95 ± 14.03 | 72.83 |

### 4.11 Bystander Effect Detection of Different Antibody Conjugated Drugs

EGFR/c-Met double-negative human breast cancer cell line MDA-MB-453 cells (purchased from Concortis) were cultured in vitro in a monolayer in DMEM/F12 medium with 10% fetal bovine serum (complete medium) at 37° C. and 5% CO₂. EGFR/c-Met double-positive human non-small cell lung cancer cell line EBC-1 cells (purchased from Nanjing Cobioer Biosciences Co., Ltd.) were cultured in vitro in a monolayer in MEM medium containing 1% NEAA, 1% sodium pyruvate, and 10% fetal calf serum (complete medium) at 37° C. and 5% CO₂. Cells were digested with trypsin-EDTA and passaged 2-3 times/week. When the cells were in the exponential growth phase, the cells were collected and counted. The two types of cells were divided into two parts, for one part, complete culture medium was used to adjust the cell density to 2.4×10⁴ cells/ml. For another portion of cells, serum-free medium was used to adjust the cell density to 1×10⁶ cells/ml. CellTrace™ Violet (1 μl) was added in the dark, and the cells were incubated at 37° C. for 20 minutes for fluorescent labeling to prepare labeled cells. Complete medium was added to terminate labeling. After washing, complete culture medium was used to adjust the labeled cell density to 2.4×10⁴ cells/ml. Three combinations of cells were prepared, as shown in Table 19. Combination one was pure negative cells, combination two was mixed cells, and combination three was pure positive cells.

TABLE 19
Combinations of cells

| | | Labeled-MDA- | | Labeled- |
|---|---|---|---|---|
| Combination | MDA-MB-453 | MB-453 | EBC-1 | EBC-1 |
| Combination 1 | 18000 | 6000 | / | / |
| (cells/well) | | | | |
| Combination 2 | / | 6000 | 18000 | / |
| (cells/well) | | | | |
| Combination 3 | / | / | 18000 | 6000 |
| (cells/well) | | | | |

After mixing, cells were added to a 24-well plate, with 1 ml of combined cell suspension added to each well and cultured overnight. After overnight culture, 10 nM hIgG1-A-14 and 49B-A-14 were added to the cells of each combination, and only complete culture medium was added to the control well for a total of 72 h. After 72 hours of incubation, the culture medium was removed from each well, trypsin was added after washing with PBS, and all cells were collected from each well. PI/RNase was added to each well to mark dead cells. The FSC/SSC/YL-1(PI)/VL-1 (CellTrace™) channel in the flow cytometer was used to detect the number of CellTrace™-labeled viable cells in each well at a fixed volume. The survival rate of labeled cells was calculated based on the measured number of labeled cells: cell survival rate %=number of cells in the sample well/number of cells in the control group×100%. The data were graphed using GraphPad Prism, and the data were displayed as mean+standard error (Mean+SEM). The results showed that after 49B-A-14 treatment, the survival rate of MDA-MB-453 cells in the mixed system was significantly lower than the survival rate of MDA-MB-453 cells in the pure negative cell system (p<0.01), indicating that 49B-A-14 has an obvious bystander effect. The specific results are shown in FIG. 9. Statistical differences between the two groups were calculated using two-tailed T-TEST, and p<0.05 was considered significant.

Unless otherwise specified, the molecular biology experimental and immunoassay methods used in the present invention are performed basically according to the methods described in J. Sambrook et al., Molecular cloning: Laboratory Manual, 2nd edition, Cold Spring Harbor Laboratory Press, 1989, and F. M. Ausubel et al., Compiled Molecular Biology Laboratory Guide, 3rd ed., John Wiley & Sons, Inc., 1995.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1. An antibody-drug conjugate comprising a structure shown in formula:
Ab-[M-L-E-D]ₓ
wherein:
Ab is a bispecific antibody or bispecific antigen-binding fragment thereof comprising a first antigen-binding domain specifically binding to c-MET and a second antigen-binding domain specifically binding to EGFR, wherein the first antigen-binding domain comprises a first light chain variable region (VL) and a first heavy chain variable region (VH), and the first VL and the first VH collectively form a domain capable of specifically binding to c-MET; and the second antigen-binding domain comprises a second VL and a second VH, and the second VL and the second VH collectively form a domain capable of specifically binding to EGFR;
M is a linker site connected to the bispecific antibody or bispecific antigen-binding fragment thereof;
L is a structural fragment connecting the linker sites M and E;
E is a structural fragment connecting L and D;
D is a cytotoxic drug or residue thereof; and
x is selected as any integer from 1 to 10.

2. (canceled)

3. The antibody-drug conjugate of claim 1, wherein the bispecific antibody or bispecific antigen-binding fragment thereof further comprises an Fc domain comprising a first Fc domain monomer and a second Fc domain monomer, and wherein the first and second Fc domain monomers comprise one or more modifications that promote heterodimerization of the Fc domain monomers.

4. (canceled)

5. The antibody-drug conjugate of claim 3, wherein the first Fc domain monomer comprises the amino acid sequence as shown in SEQ ID NO: 49 or 51, and the second Fc domain monomer comprises the amino acid sequence as shown in SEQ ID NO: 50 or 52.

6. The antibody-drug conjugate of claim 3, wherein the first antigen-binding domain and the second antigen-binding domain are each linked to one of the first and second Fc domain monomers of the Fc domain.

7.-8. (canceled)

9. The antibody-drug conjugate of claim 1, wherein:
the first VL comprises:
(i) a CDR-L1 comprising the amino acid sequence as shown in SEQ ID NO: 34, a CDR-L2 comprising the amino acid sequence as shown in SEQ ID NO: 36, and a CDR-L3 comprising the amino acid sequence as shown in SEQ ID NO: 38; or
(ii) a CDR-L1 comprising the amino acid sequence as shown in SEQ ID NO: 35, a CDR-L2 comprising the amino acid sequence as shown in SEQ ID NO: 37, and a CDR-L3 comprising the amino acid sequence as shown in SEQ ID NO: 38; and/or
the first VH comprises:
(i) a CDR-H1 comprising the amino acid sequence as shown in SEQ ID NO: 39, a CDR-H2 comprising the amino acid sequence as shown in SEQ ID NO: 43, and a CDR-H3 comprising the amino acid sequence as shown in SEQ ID NO: 47;
(ii) a CDR-H1 comprising the amino acid sequence as shown in SEQ ID NO: 40, a CDR-H2 comprising the amino acid sequence as shown in SEQ ID NO: 44, and a CDR-H3 comprising the amino acid sequence as shown in SEQ ID NO: 47;
(iii) a CDR-H1 comprising the amino acid sequence as shown in SEQ ID NO: 42, a CDR-H2 comprising the amino acid sequence as shown in SEQ ID NO: 46, and a CDR-H3 comprising the amino acid sequence as shown in SEQ ID NO: 47; or
(iv) a CDR-H1 comprising the amino acid sequence as shown in SEQ ID NO: 41, a CDR-H2 comprising the amino acid sequence as shown in SEQ ID NO: 45, and a CDR-H3 comprising the amino acid sequence as shown in SEQ ID NO: 48.

10. The antibody-drug conjugate of claim 1, wherein:
the first VL comprises the amino acid sequence as shown in SEQ ID NO: 17 or 59 and/or the first VH comprises the amino acid sequence as shown in SEQ ID NO: 18 or 60; or
the first VL comprises the amino acid sequence as shown in SEQ ID NO: 17, the first VH comprises the amino acid sequence as shown in SEQ ID NO: 18; or the first VL comprises the amino acid sequence as shown in SEQ ID NO: 59, the first VH comprises the amino acid sequence as shown in SEQ ID NO: 60.

11. (canceled)

12. The antibody-drug conjugate of claim 1, wherein:
the second VL comprises:
(i) a CDR-L1 comprising the amino acid sequence as shown in SEQ ID NO: 19, a CDR-L2 comprising the amino acid sequence as shown in SEQ ID NO: 21, and a CDR-L3 comprising the amino acid sequence as shown in SEQ ID NO: 23; or
(ii) a CDR-L1 comprising the amino acid sequence as shown in SEQ ID NO: 20, a CDR-L2 comprising the amino acid sequence as shown in SEQ ID NO: 22, and a CDR-L3 comprising the amino acid sequence as shown in SEQ ID NO: 23; and/or
the second VH comprises:
(i) a CDR-H1 comprising the amino acid sequence as shown in SEQ ID NO: 24, a CDR-H2 comprising the amino acid sequence as shown in SEQ ID NO: 28, and a CDR-H3 comprising the amino acid sequence as shown in SEQ ID NO: 32;
(ii) a CDR-H1 comprising the amino acid sequence as shown in SEQ ID NO: 25, a CDR-H2 comprising the amino acid sequence as shown in SEQ ID NO: 29, and a CDR-H3 comprising the amino acid sequence as shown in SEQ ID NO: 32;
(iii) a CDR-H1 comprising the amino acid sequence as shown in SEQ ID NO: 27, a CDR-H2 comprising the amino acid sequence as shown in SEQ ID NO: 31, and a CDR-H3 comprising the amino acid sequence as shown in SEQ ID NO: 32; or
(iv) a CDR-H1 comprising the amino acid sequence as shown in SEQ ID NO: 26, a CDR-H2 comprising the amino acid sequence as shown in SEQ ID NO: 30, and a CDR-H3 comprising the amino acid sequence as shown in SEQ ID NO: 33.

13. The antibody-drug conjugate of claim 1, wherein the second VL comprises the amino acid sequence as shown in SEQ ID NO: 15, and/or the second VH comprises the amino acid sequence as shown in SEQ ID NO: 16.

14. The antibody-drug conjugate of claim 1, wherein the first antigen-binding domain is a Fab, and the second antigen-binding domain is an scFv.

15. The antibody-drug conjugate of claim 14, wherein the bispecific antibody comprises a polypeptide chain I-A, a polypeptide chain I-B and a polypeptide chain I-C; wherein the polypeptide chain I-A comprises the first VL and a light chain constant region; the polypeptide chain I-B comprises: the first VH, a heavy chain CH1 region and the first Fc domain monomer (or the second Fc domain monomer); and/or the polypeptide chain I-C comprises: the second VL, the second VH and the second Fc domain monomer (or the first Fc domain monomer).

16.-18. (canceled)

19. The antibody-drug conjugate of claim 15, wherein the light chain constant region comprises the amino acid sequence as shown in SEQ ID NO: 53 or SEQ ID NO: 63, and/or the heavy chain CH1 region comprises the amino acid sequence as shown in SEQ ID NO: 54.

20. The antibody-drug conjugate of claim 15, wherein the polypeptide chain I-A comprises the amino acid sequence as shown in SEQ ID NO: 1, the polypeptide chain I-B comprises the amino acid sequence as shown in SEQ ID NO: 2 or 9, and/or the polypeptide chain I-C comprises the amino acid sequence as shown in SEQ ID NO: ID NO: 3 or 10.

21. The antibody-drug conjugate of claim 1, wherein the first antigen-binding domain is an scFv, and the second antigen-binding domain is a Fab.

22. The antibody-drug conjugate of claim 21, wherein the bispecific antibody comprises a polypeptide chain II-A, a polypeptide chain II-B and a polypeptide chain II-C; wherein the polypeptide chain II-A comprises: the second VL and a light chain constant region; the polypeptide chain II-B comprises the second VH, a heavy chain CH1 region and the first Fc domain monomer (or the second Fc domain monomer); and/or the polypeptide chain II-C comprises: the first VL, the first VH and the second Fc domain monomer (or the first Fc domain monomer).

23.-25. (canceled)

26. The antibody-drug conjugate of claim 22, wherein the light chain constant region comprises the amino acid sequence as shown in SEQ ID NO: 53 or SEQ ID NO: 63, and/or the heavy chain CH1 region comprises the amino acid sequence as shown in SEQ ID NO: 54.

27. The antibody-drug conjugate of claim 22, wherein the polypeptide chain II-A comprises the amino acid sequence as shown in SEQ ID NO: 4, the polypeptide chain II-B comprises the amino acid sequence as shown in SEQ ID NO: 5 or 7, and/or the polypeptide chain II-C comprises the amino acid sequence as shown in SEQ ID NO: 6 or 8.

28. The antibody-drug conjugate of claim 1, wherein the first antigen-binding domain is a Fab, and the second antigen-binding domain is an scFab.

29. The antibody-drug conjugate of claim 28, wherein the bispecific antibody comprises a polypeptide chain III-A, a polypeptide chain III-B and a polypeptide chain III-C; wherein the polypeptide chain III-A comprises the first VL and a light chain constant region; the polypeptide chain III-B comprises: the first VH, a heavy chain CH1 region and the first Fc domain monomer (or the second Fc domain monomer), the polypeptide chain III-C comprises: the second VL, a light chain constant region, the second VH, a heavy chain CH1 region and the second Fc domain monomer (or the first Fc domain monomer).

30.-32. (canceled)

33. The antibody-drug conjugate of claim 29, wherein the light chain constant region comprises the amino acid sequence as shown in SEQ ID NO: 53 or SEQ ID NO: 63, and/or the CH1 region of the heavy chain comprises the amino acid sequence as shown in SEQ ID NO: 54.

34. The antibody-drug conjugate of claim 1, wherein the first antigen-binding domain is an scFab, and the second antigen-binding domain is a Fab.

35. The antibody-drug conjugate of claim 34, wherein the bispecific antibody comprises a polypeptide chain IV-A, a polypeptide chain IV-B and a polypeptide chain IV-C; wherein the polypeptide chain IV-A comprises the second VL and a light chain constant region; the polypeptide chain IV-B comprises: the second VH, a heavy chain CH1 region and the first Fc domain monomer (or the second Fc domain monomer); the polypeptide chain IV-C comprises: the first VL, a light chain constant region, the first VH, a heavy chain CH1 region and the second Fc domain monomer (or the first Fc domain monomer).

36.-38. (canceled)

39. The antibody-drug conjugate of claim 35, wherein the light chain constant region comprises the amino acid sequence as shown in SEQ ID NO: 53 or SEQ ID NO: 63, and/or the heavy chain CH1 region comprises the amino acid sequence as shown in SEQ ID NO: 54.

40. The antibody-drug conjugate of claim 35, wherein the polypeptide chain IV-A comprises the amino acid sequence as shown in SEQ ID NO: 4, the polypeptide chain IV-B comprises the amino acid sequence as shown in SEQ ID NO: 7, and/or the polypeptide chain IV-C comprises the amino acid sequence as shown in SEQ ID NO: 11.

41. The antibody-drug conjugate of claim 1, wherein the first antigen-binding domain and the second antigen-binding domain are Fabs, and the Fab of the second antigen-binding domain comprises domain swaps in a form of CrossMab.

42. The antibody-drug conjugate of claim 41, wherein the bispecific antibody comprises a polypeptide chain V-A, a polypeptide chain V-B, a polypeptide chain V-C and a polypeptide chain V-D; wherein the polypeptide chain V-A comprises the first VL and a light chain constant region; the polypeptide chain V-B comprises: the first VH, a heavy chain CH1 region and the first Fc domain monomer (or the second Fc domain monomer), and the polypeptide chain V-C comprises: the second VH, a light chain constant region and the second Fc domain monomer (or the first Fc domain monomer), the polypeptide chain V-D comprises: the second VL and a heavy chain CH1 region.

43.-45. (canceled)

46. The antibody-drug conjugate of claim 42, wherein the light chain constant region comprises the amino acid sequence as shown in SEQ ID NO: 53 or SEQ ID NO: 63, and/or the heavy chain CH1 region comprises the amino acid sequence as shown in SEQ ID NO: 54.

47. The antibody-drug conjugate of claim 42, wherein the polypeptide chain V-A comprises the amino acid sequence as shown in SEQ ID NO: 1, the polypeptide chain V-B comprises the amino acid sequence as shown in SEQ ID NO: 9; the polypeptide chain V-C comprises the amino acid sequence as shown in SEQ ID NO: 13, and/or the sequence of the polypeptide chain V-D is shown in SEQ ID NO: 12.

48. The antibody-drug conjugate of claim 1, wherein the first antigen-binding domain and the second antigen-binding domain are Fabs, and the Fab of the first antigen-binding domain comprises domain swaps in a form of CrossMab.

49. The antibody-drug conjugate of claim 48, wherein the bispecific antibody comprises a polypeptide chain VI-A, a polypeptide chain VI-B, a polypeptide chain VI-C and a polypeptide chain VI-D; wherein the polypeptide chain VI-A comprises the second VL and a light chain constant region; the polypeptide chain VI-B comprises: the second VH, a heavy chain CH1 region and the first Fc domain monomer (or the second Fc domain monomer); the polypeptide chain VI-C comprises: the first VH, a light chain constant region and the second Fc domain monomer (or the first Fc domain monomer); the polypeptide chain VI-D comprises: the first VL and a heavy chain CH1 region.

50.-52. (canceled)

53. The antibody-drug conjugate of claim 49, wherein the light chain constant region comprises the amino acid sequence as shown in SEQ ID NO: 53 or SEQ ID NO: 63, and/or the heavy chain CH1 region comprises the amino acid sequence as shown in SEQ ID NO: 54.

54. The antibody-drug conjugate of claim 1, wherein both the first antigen-binding domain and the second antigen-binding domain are scFvs.

55. The antibody-drug conjugate of claim 54, wherein the bispecific antibody comprises a polypeptide chain VII-A and polypeptide chain VII-B; wherein the polypeptide chain VII-A comprises: the first VL, the first VH and the first Fc domain monomer (or the second Fc domain monomer), the polypeptide chain VII-B comprises: the second VL, the second VH and the second Fc domain monomer (or the first Fc domain monomer).

56.-59. (canceled)

60. The antibody-drug conjugate of claim 55, wherein the polypeptide chain VII-A comprises the amino acid sequence as shown in SEQ ID NO: 14, and/or the polypeptide chain VII-B comprises the amino acid sequence as shown in SEQ ID NO: 8.

61. The antibody-drug conjugate of claim 1, wherein the bispecific antibody or antigen-binding fragment thereof comprises:
(1) a polypeptide chain I-A comprising the amino acid sequence shown in SEQ ID NO: 1, a polypeptide chain I-B comprising the amino acid sequence shown in SEQ ID NO: 2, and a polypeptide chain I-C comprising the amino acid sequence shown in SEQ ID NO: 3;
(2) a polypeptide chain I-A comprising the amino acid sequence shown in SEQ ID NO: 1, a polypeptide chain I-B comprising the amino acid sequence shown in SEQ ID NO: 9, and a polypeptide chain I-C comprising the amino acid sequence shown in SEQ ID NO: 10;
(3) a polypeptide chain II-A comprising the amino acid sequence shown in SEQ ID NO: 4, a polypeptide chain II-B comprising the amino acid sequence shown in SEQ ID NO: 5, and a polypeptide chain II-C comprising the amino acid sequence shown in SEQ ID NO: 6;
(4) a polypeptide chain II-A comprising the amino acid sequence shown in SEQ ID NO: 4, a polypeptide chain II-B comprising the amino acid sequence shown in SEQ ID NO: 7, and a polypeptide chain II-C comprising the amino acid sequence shown in SEQ ID NO: 8;
(5) a polypeptide chain IV-A comprising the amino acid sequence shown in SEQ ID NO: 4, a polypeptide chain IV-B comprising the amino acid sequence shown in SEQ ID NO: 7, and a polypeptide chain IV-C comprising the amino acid sequence shown in SEQ ID NO: 11;
(6) a polypeptide chain V-A comprising the amino acid sequence shown in SEQ ID NO: 1, a polypeptide chain V-B comprising the amino acid sequence shown in SEQ ID NO: 9, a polypeptide chain V-C comprising the amino acid sequence shown in SEQ ID NO: 13, and a polypeptide chain V-D comprising the amino acid sequence as shown in SEQ ID NO: 12; or
(7) a polypeptide chain VII-A comprising the amino acid sequence shown in SEQ ID NO: 14, and a polypeptide chain VII-B comprising the amino acid sequence shown in SEQ ID NO: 8.

62.-63. (canceled)

64. The antibody-drug conjugate of claim 1, wherein M comprises 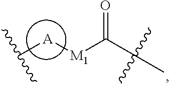
and wherein ring A is a 5-6 membered aliphatic heterocyclic ring or a 5-20 membered aromatic ring system, or a polycyclic ring formed by connecting more than one 6-membered aromatic heterocyclic ring and a benzene ring through a single bond or by connecting more than one 6-membered heteroaromatic rings through single bonds, and wherein the aliphatic heterocyclic ring and aromatic ring system are optionally replaced by one or more groups independently selected from the group consisting of oxo (═O), halogen, cyano, amino, carboxyl, thiol, and C₁₋₆ alkyl; and M₁ is selected from single bond, C₁₋₂₀ alkylene, C₂₋₂₀ alkenylene, C₂₋₂₀ alkynylene, or amine group.

65.-66. (canceled)

67. The antibody-drug conjugate of claim 1, wherein M is selected from

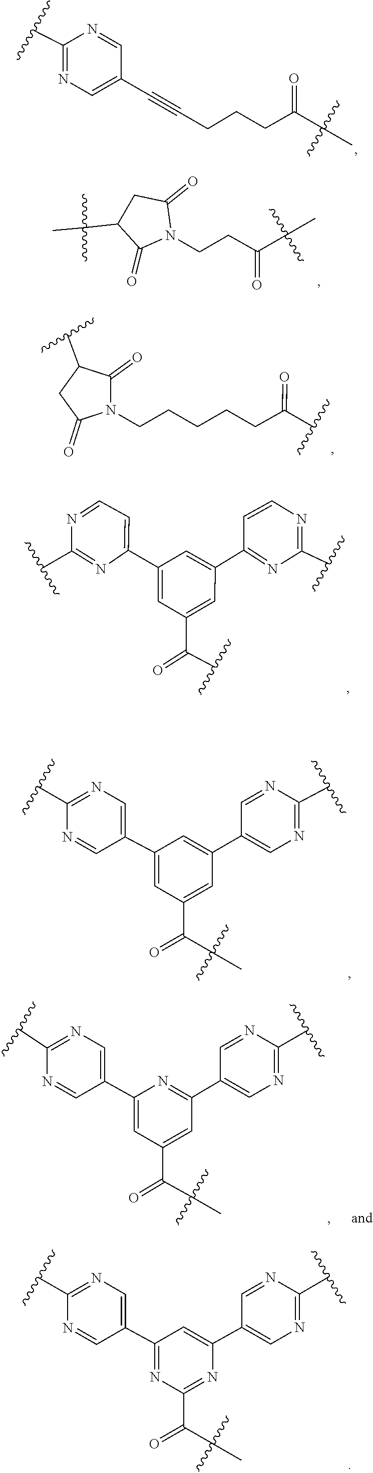

68.-70. (canceled)

71. The antibody-drug conjugate of claim 1, wherein L is selected from a structure comprising one or more of the following: C₁₋₆ alkylene, —N(R′)—, carbonyl, —O—, natural amino acids or unnatural amino acids and their analogs selected from Ala, Arg, Asn, Asp, Cit, Cys, Gln, Glu, Gly, His, Ile, Leu, Lys, Met, Phe, Pro, Ser, Thr, Trp, Tyr, Val, Lys (COCH₂CH₂(OCH₂CH₂)ₛOCH₃), and peptides comprising 1, 2, 3, or 4 amino acids 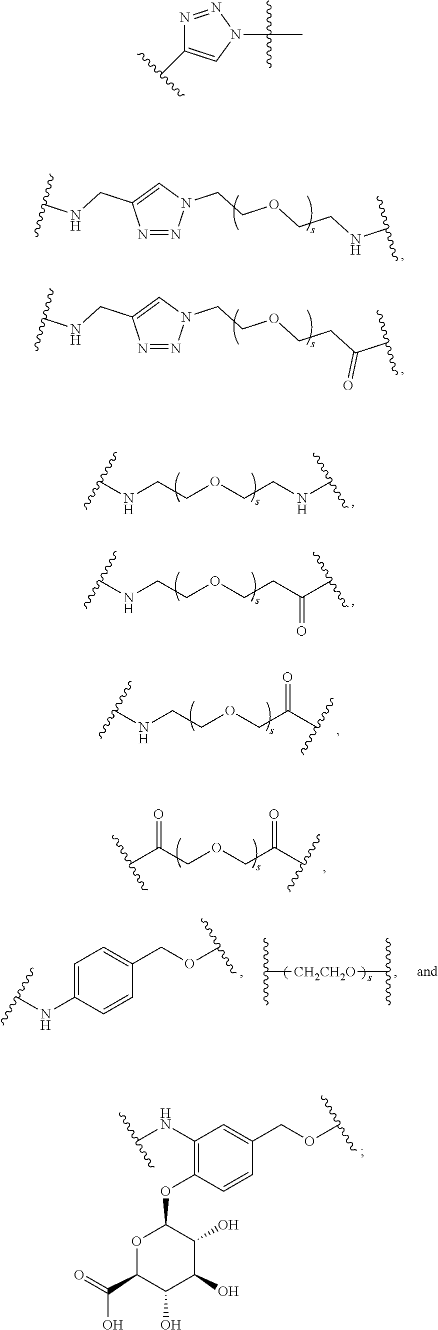
wherein R′ represents hydrogen, C₁₋₆ alkyl, or a polyethylene glycol fragment containing 1-10 ethylene oxide units; and s is selected from an integer of 1-20.

72. The antibody-drug conjugate of claim 71, wherein the peptide is selected from Ala-Ala, Ala-Lys, Ala-Lys(Ac), Ala-Pro, Gly-Glu, Gly-Gly, Phe-Lys, Phe-Lys(Ac), Val-Ala, Val-Lys, Val-Lys(Ac), Val-Cit, Ala-Ala-Ala, Ala-Ala-Asn, Leu-Ala-Glu, Gly-Gly-Arg, Gly-Glu-Gly, Gly-Gly-Gly, Gly-Ser-Lys, Glu-Val-Ala, Glu-Val-Cit, Ser-Ala-Pro, Val-Leu-Lys, Val-Lys-Ala, Val-Lys-Gly, Gly-Gly-Phe-Gly (GGFG (SEQ ID NO: 71)), Gly-Gly-Val-Ala (GGVA (SEQ ID NO: 72), Gly-Phe-Leu-Gly (GFLG (SEQ ID NO: 73)), Glu-Ala-Ala-Ala (EAAA (SEQ ID NO: 74)), and Gly-Gly-Gly-Gly-Gly (GGGGG (SEQ ID NO: 75).

73. (canceled)

74. The antibody-drug conjugate of claim 71, wherein L is selected from a structure consisting of one or more of the following:

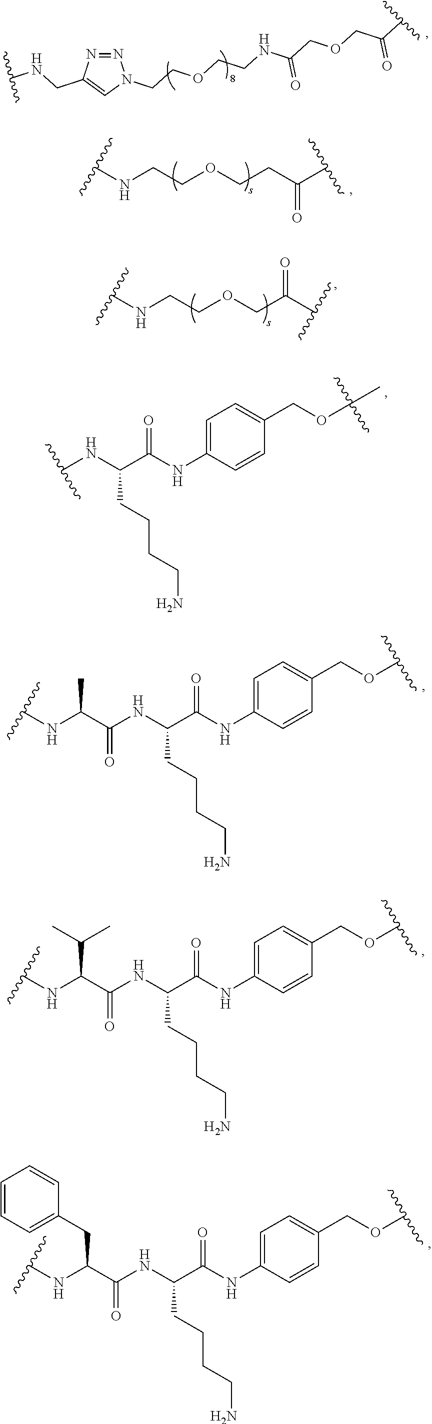

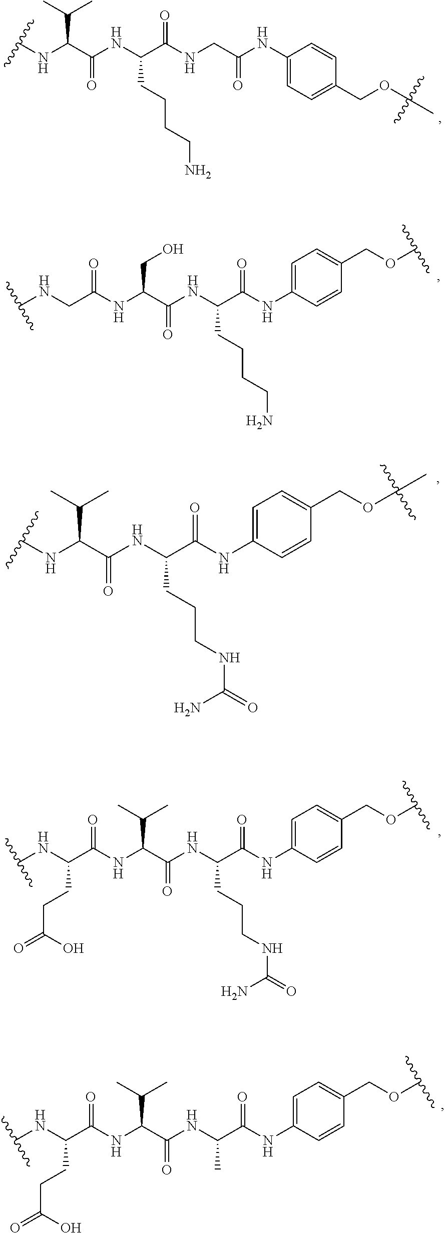 [SMILES: *N[C@H](C(=O)N[C@@H](CCCCN)C(=O)NCC(=O)Nc1ccc(CO*)cc1)C(C)C ; *NCC(=O)N[C@@H](CO)C(=O)N[C@@H](CCCCN)C(=O)Nc1ccc(CO*)cc1 ; *N[C@H](C(=O)N[C@@H](CCCNC(N)=O)C(=O)Nc1ccc(CO*)cc1)C(C)C ; *N[C@@H](CCC(=O)O)C(=O)N[C@H](C(=O)N[C@@H](CCCNC(N)=O)C(=O)Nc1ccc(CO*)cc1)C(C)C ; *N[C@@H](CCC(=O)O)C(=O)N[C@H](C(=O)N[C@@H](C)C(=O)Nc1ccc(CO*)cc1)C(C)C]

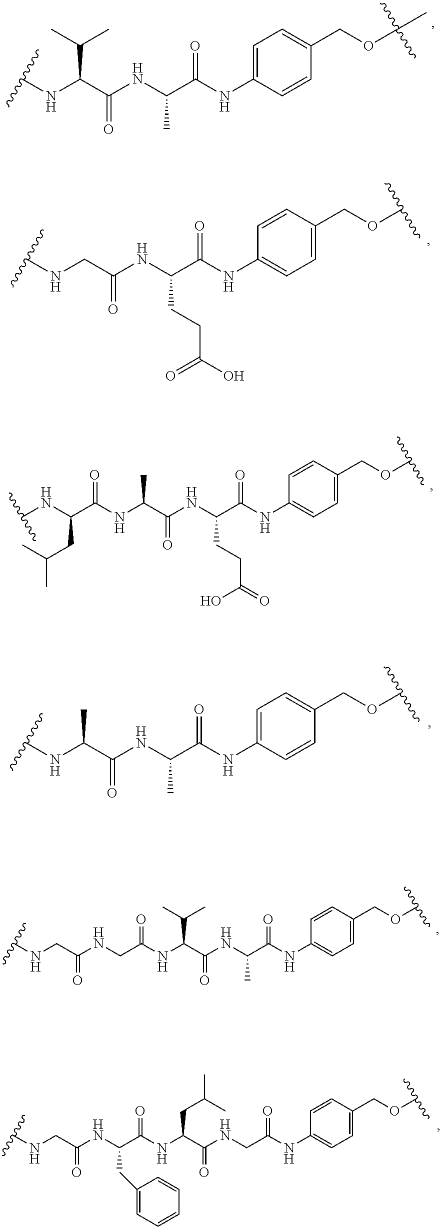 [SMILES: *N[C@H](C(=O)N[C@@H](C)C(=O)Nc1ccc(CO*)cc1)C(C)C ; *NCC(=O)N[C@@H](CCC(=O)O)C(=O)Nc1ccc(CO*)cc1 ; *N[C@H](CC(C)C)C(=O)N[C@@H](C)C(=O)N[C@@H](CCC(=O)O)C(=O)Nc1ccc(CO*)cc1 ; *N[C@@H](C)C(=O)N[C@@H](C)C(=O)Nc1ccc(CO*)cc1 ; *NCC(=O)NCC(=O)N[C@H](C(=O)N[C@@H](C)C(=O)Nc1ccc(CO*)cc1)C(C)C ; *NCC(=O)N[C@@H](Cc1ccccc1)C(=O)N[C@@H](CC(C)C)C(=O)NCC(=O)Nc1ccc(CO*)cc1]

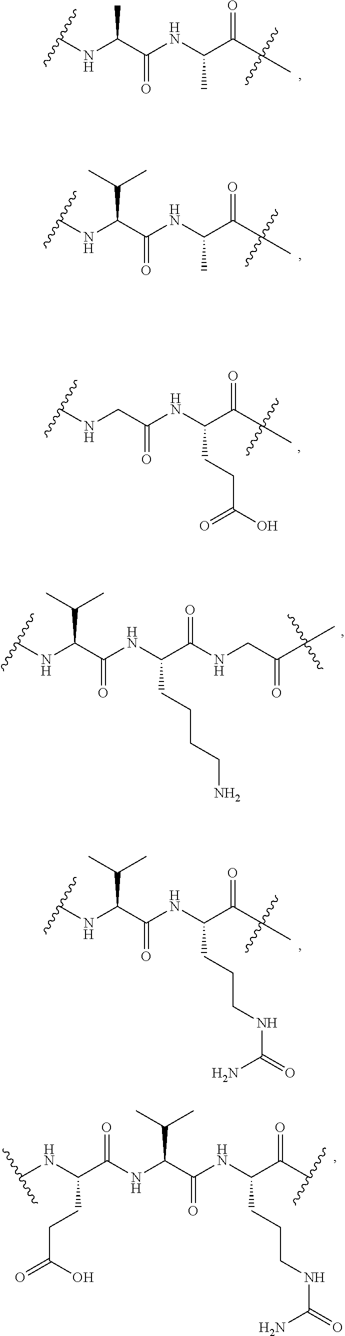 [SMILES: *N[C@@H](C)C(=O)N[C@@H](C)C(*)=O ; *N[C@H](C(=O)N[C@@H](C)C(*)=O)C(C)C ; *NCC(=O)N[C@@H](CCC(=O)O)C(*)=O ; *N[C@H](C(=O)N[C@@H](CCCCN)C(=O)NCC(*)=O)C(C)C ; *N[C@H](C(=O)N[C@@H](CCCNC(N)=O)C(*)=O)C(C)C ; *N[C@@H](CCC(=O)O)C(=O)N[C@H](C(=O)N[C@@H](CCCNC(N)=O)C(*)=O)C(C)C]

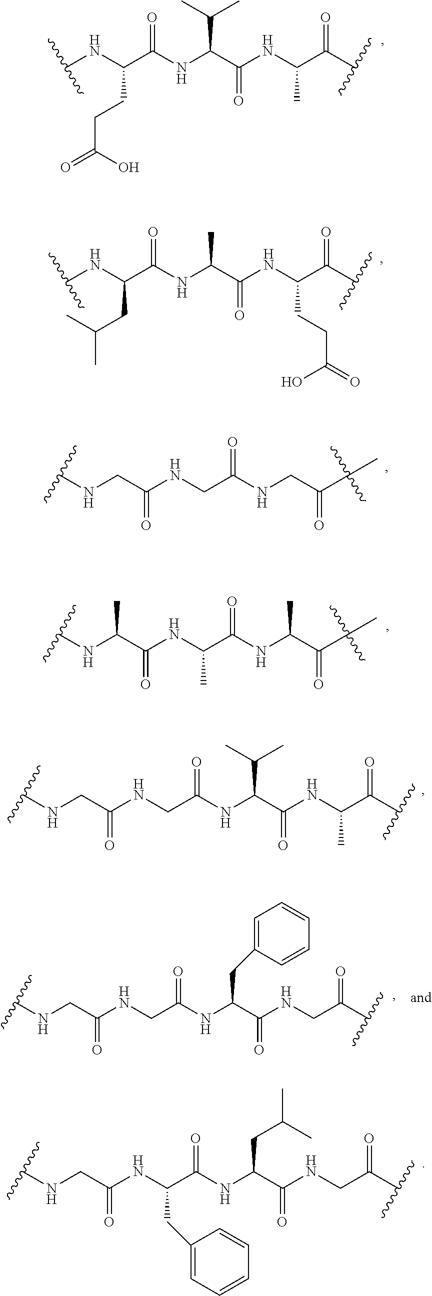

75.-78. (canceled)

79. The antibody-drug conjugate of claim 1, wherein E is a single bond or selected from the following structures: —NHCH₂—,

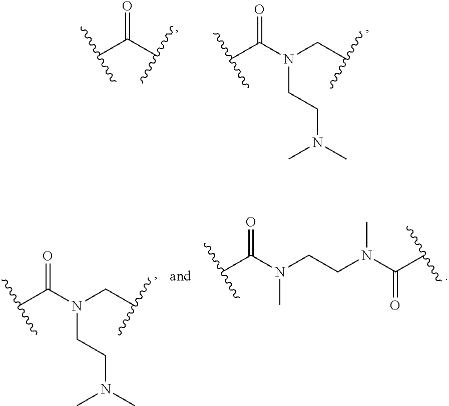

80.-83. (canceled)

84. The antibody-drug conjugate of claim 1, wherein 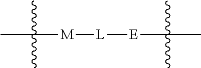
is selected from the following structures:

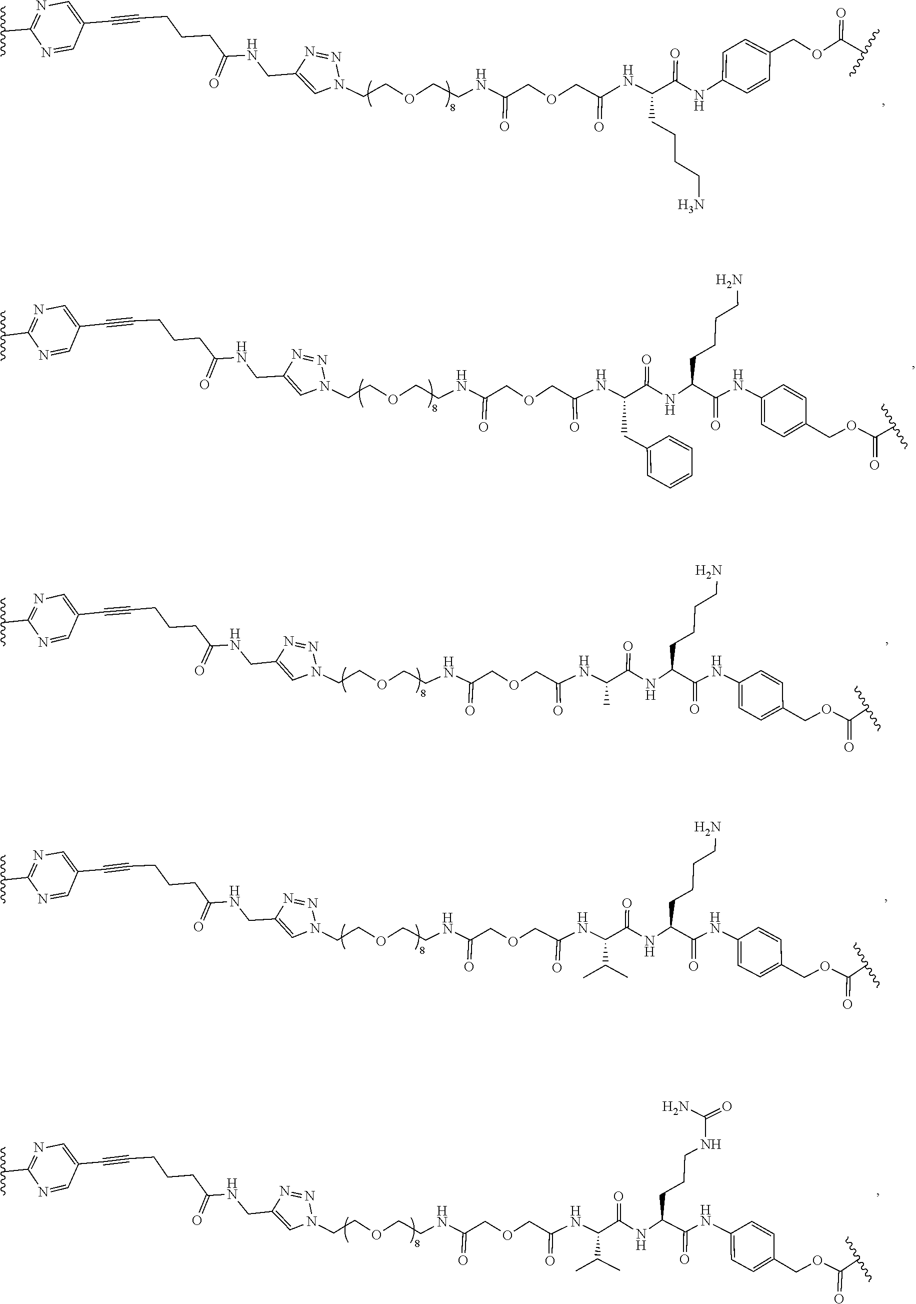

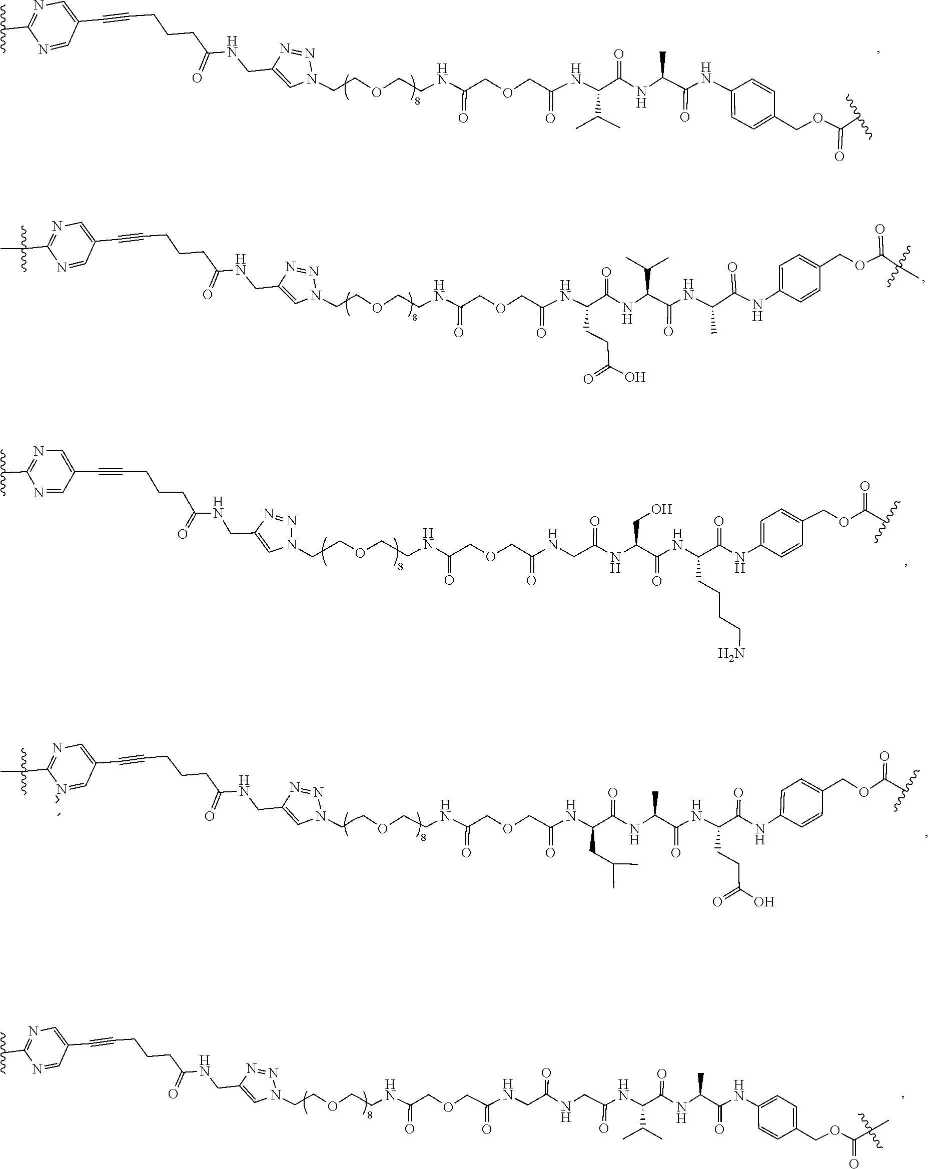

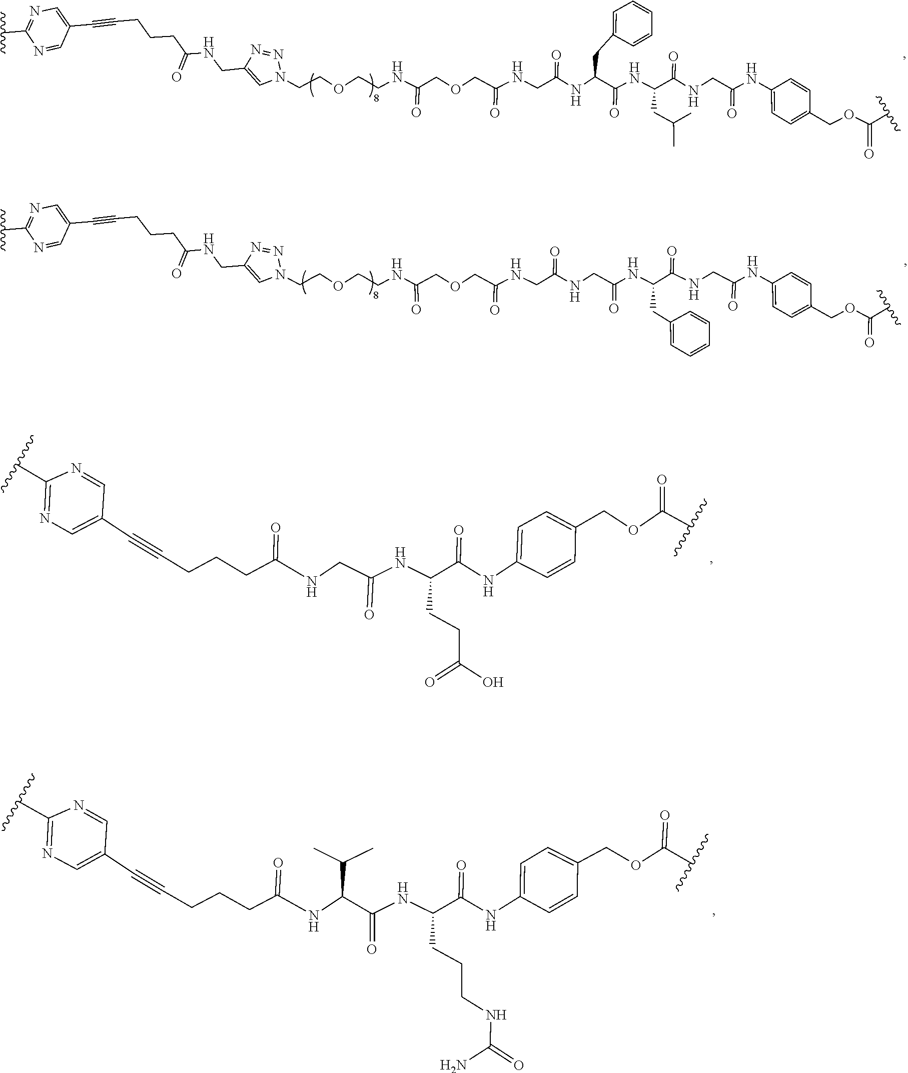

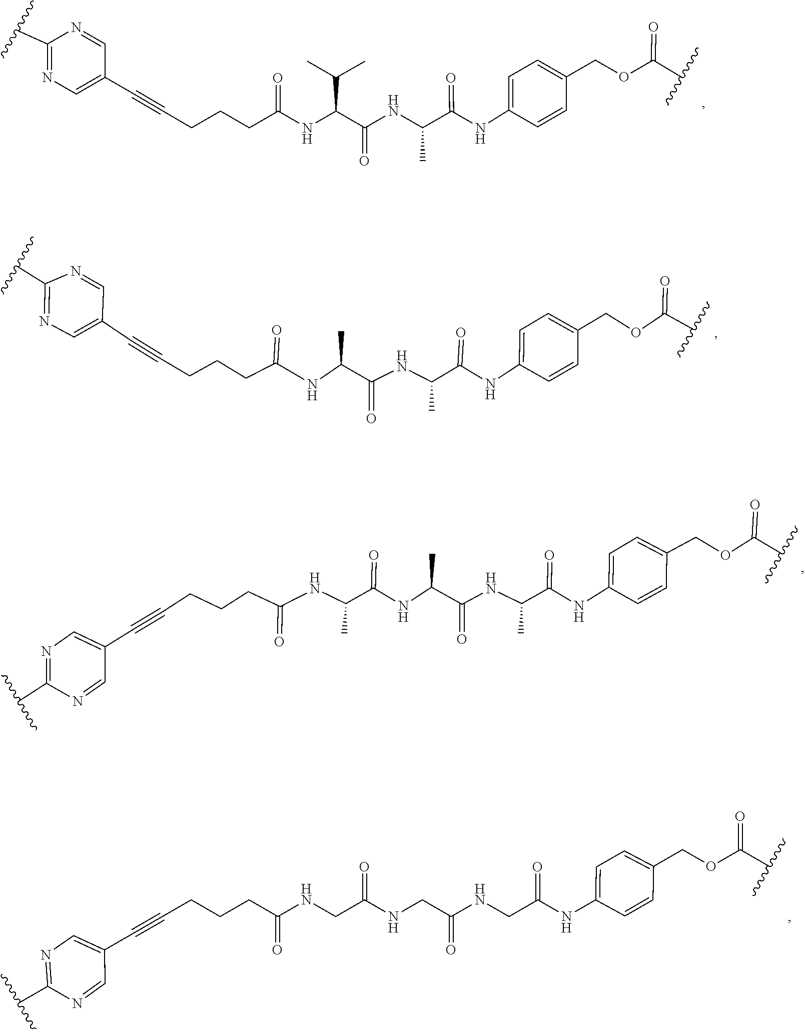

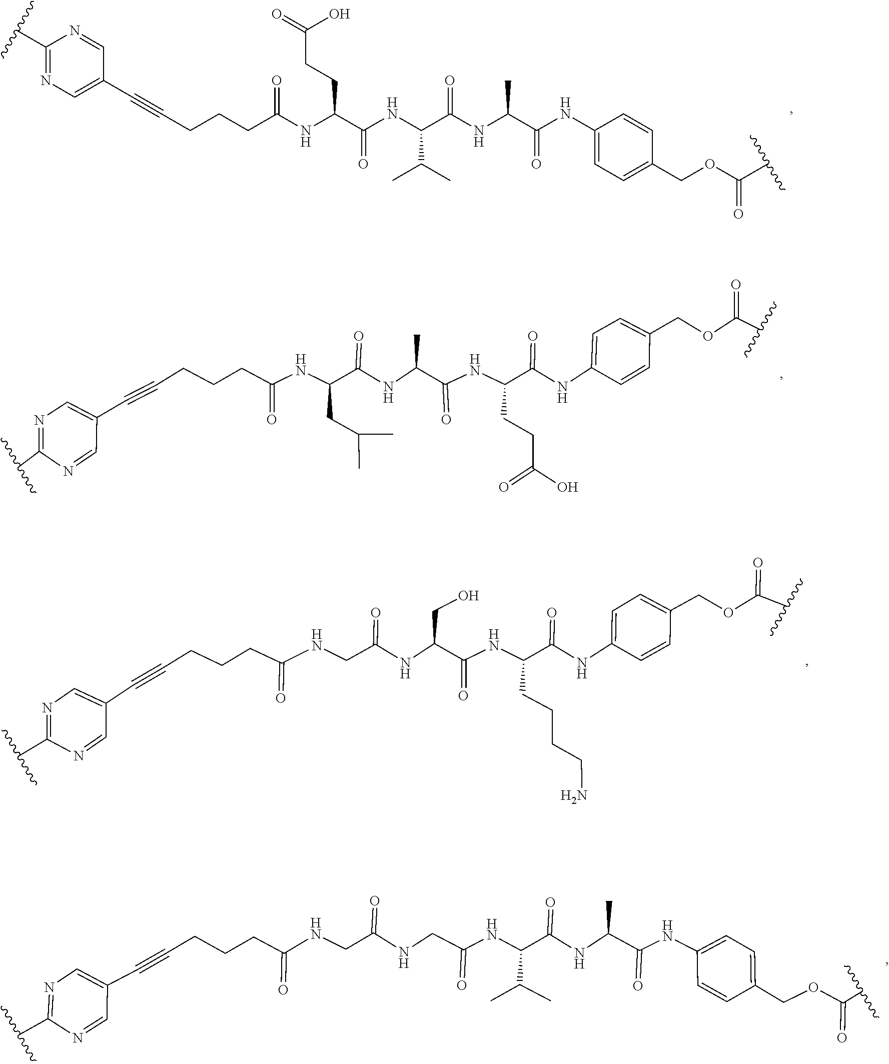 [SMILES: *C(=O)OCc1ccc(NC(=O)[C@H](C)NC(=O)[C@@H](NC(=O)[C@H](CCC(=O)O)NC(=O)CCCC#Cc2cnc(*)nc2)C(C)C)cc1 ; *C(=O)OCc1ccc(NC(=O)[C@H](CCC(=O)O)NC(=O)[C@H](C)NC(=O)[C@@H](CC(C)C)NC(=O)CCCC#Cc2cnc(*)nc2)cc1 ; *C(=O)OCc1ccc(NC(=O)[C@H](CCCCN)NC(=O)[C@H](CO)NC(=O)CNC(=O)CCCC#Cc2cnc(*)nc2)cc1 ; *C(=O)OCc1ccc(NC(=O)[C@H](C)NC(=O)[C@@H](NC(=O)CNC(=O)CNC(=O)CCCC#Cc2cnc(*)nc2)C(C)C)cc1]

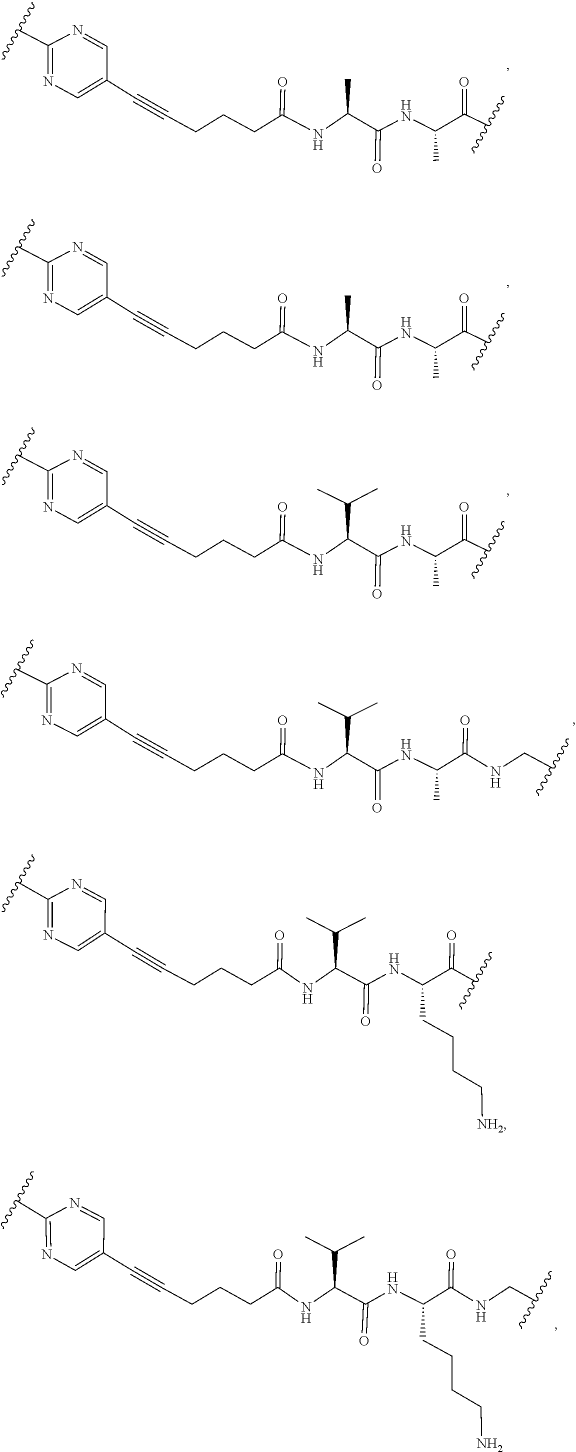 [SMILES: *C(=O)[C@H](C)NC(=O)[C@H](C)NC(=O)CCCC#Cc1cnc(*)nc1 ; *C(=O)[C@H](C)NC(=O)[C@H](C)NC(=O)CCCC#Cc1cnc(*)nc1 ; *C(=O)[C@H](C)NC(=O)[C@@H](NC(=O)CCCC#Cc1cnc(*)nc1)C(C)C ; *CNC(=O)[C@H](C)NC(=O)[C@@H](NC(=O)CCCC#Cc1cnc(*)nc1)C(C)C ; *C(=O)[C@H](CCCCN)NC(=O)[C@@H](NC(=O)CCCC#Cc1cnc(*)nc1)C(C)C ; *CNC(=O)[C@H](CCCCN)NC(=O)[C@@H](NC(=O)CCCC#Cc1cnc(*)nc1)C(C)C]

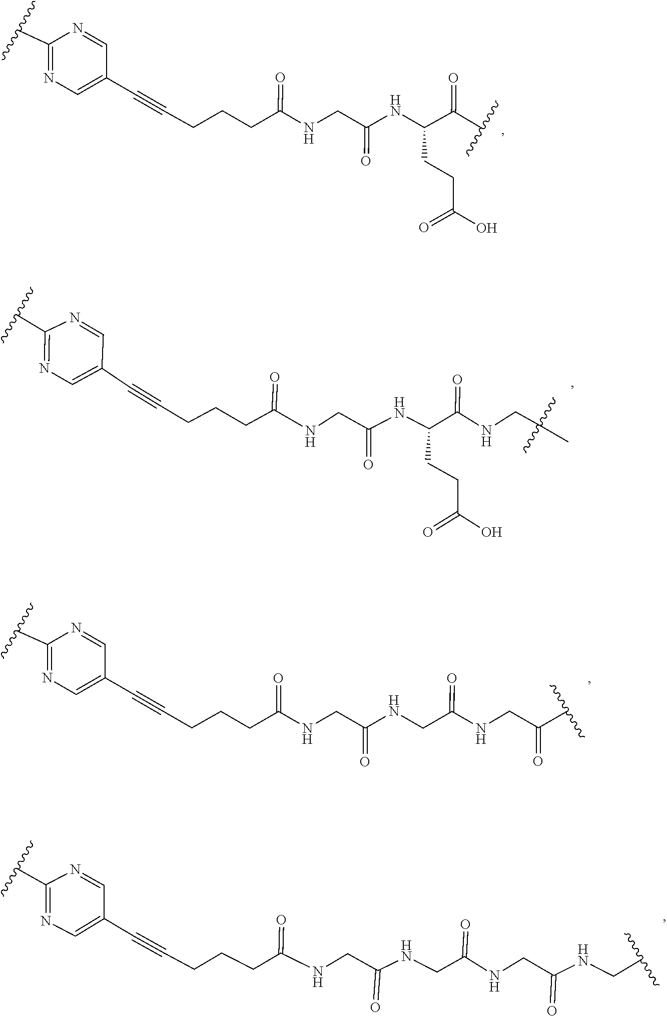 [SMILES: *C(=O)[C@H](CCC(=O)O)NC(=O)CNC(=O)CCCC#Cc1cnc(*)nc1 ; *CNC(=O)[C@H](CCC(=O)O)NC(=O)CNC(=O)CCCC#Cc1cnc(*)nc1 ; *C(=O)CNC(=O)CNC(=O)CNC(=O)CCCC#Cc1cnc(*)nc1 ; *CNC(=O)CNC(=O)CNC(=O)CNC(=O)CCCC#Cc1cnc(*)nc1]

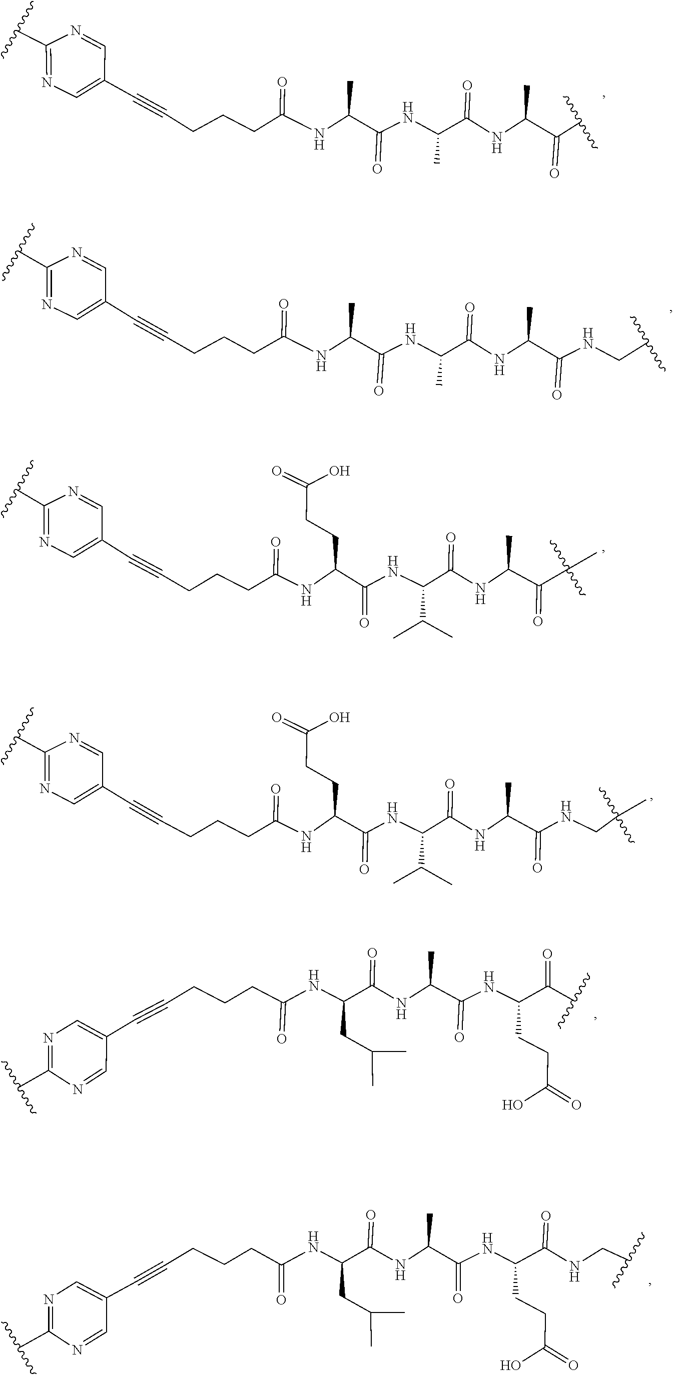 [SMILES: *C(=O)[C@H](C)NC(=O)[C@H](C)NC(=O)[C@H](C)NC(=O)CCCC#Cc1cnc(*)nc1 ; *CNC(=O)[C@H](C)NC(=O)[C@H](C)NC(=O)[C@H](C)NC(=O)CCCC#Cc1cnc(*)nc1 ; *C(=O)[C@H](C)NC(=O)[C@@H](NC(=O)[C@H](CCC(=O)O)NC(=O)CCCC#Cc1cnc(*)nc1)C(C)C ; *CNC(=O)[C@H](C)NC(=O)[C@@H](NC(=O)[C@H](CCC(=O)O)NC(=O)CCCC#Cc1cnc(*)nc1)C(C)C ; *C(=O)[C@H](CCC(=O)O)NC(=O)[C@H](C)NC(=O)[C@@H](CC(C)C)NC(=O)CCCC#Cc1cnc(*)nc1 ; *CNC(=O)[C@H](CCC(=O)O)NC(=O)[C@H](C)NC(=O)[C@@H](CC(C)C)NC(=O)CCCC#Cc1cnc(*)nc1]

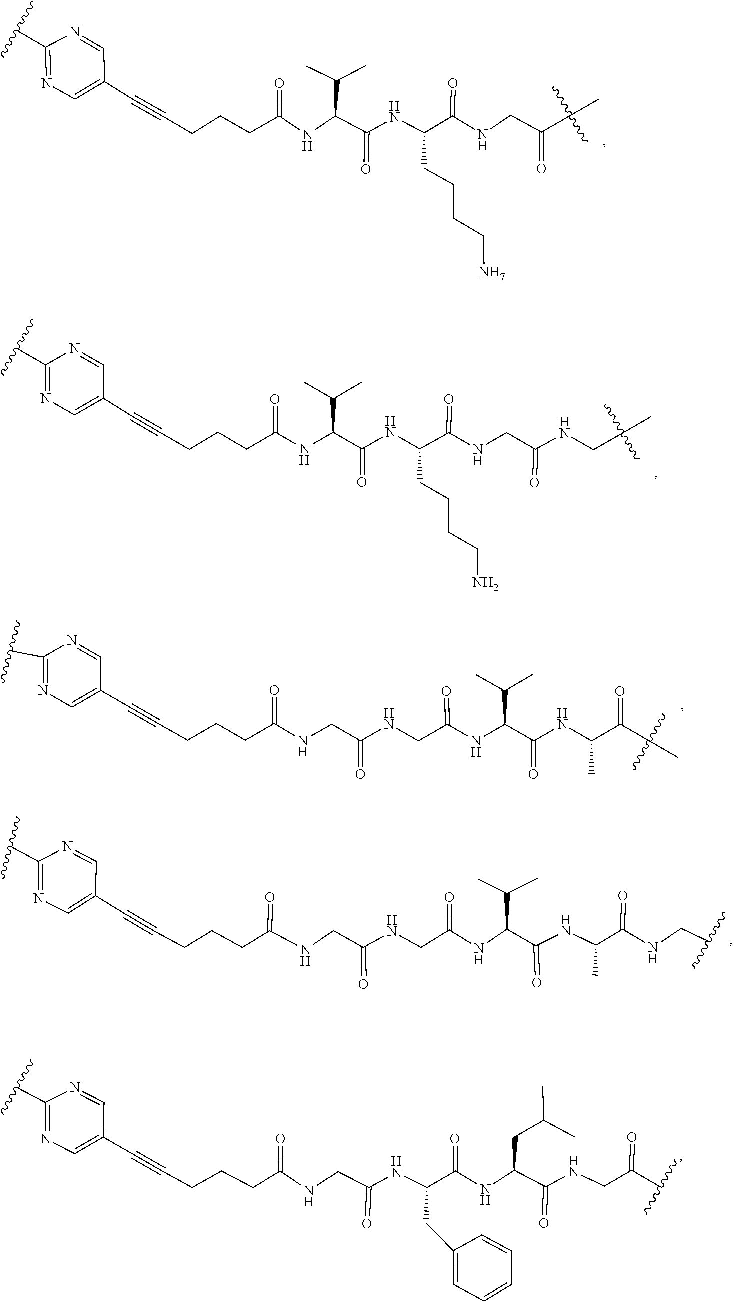

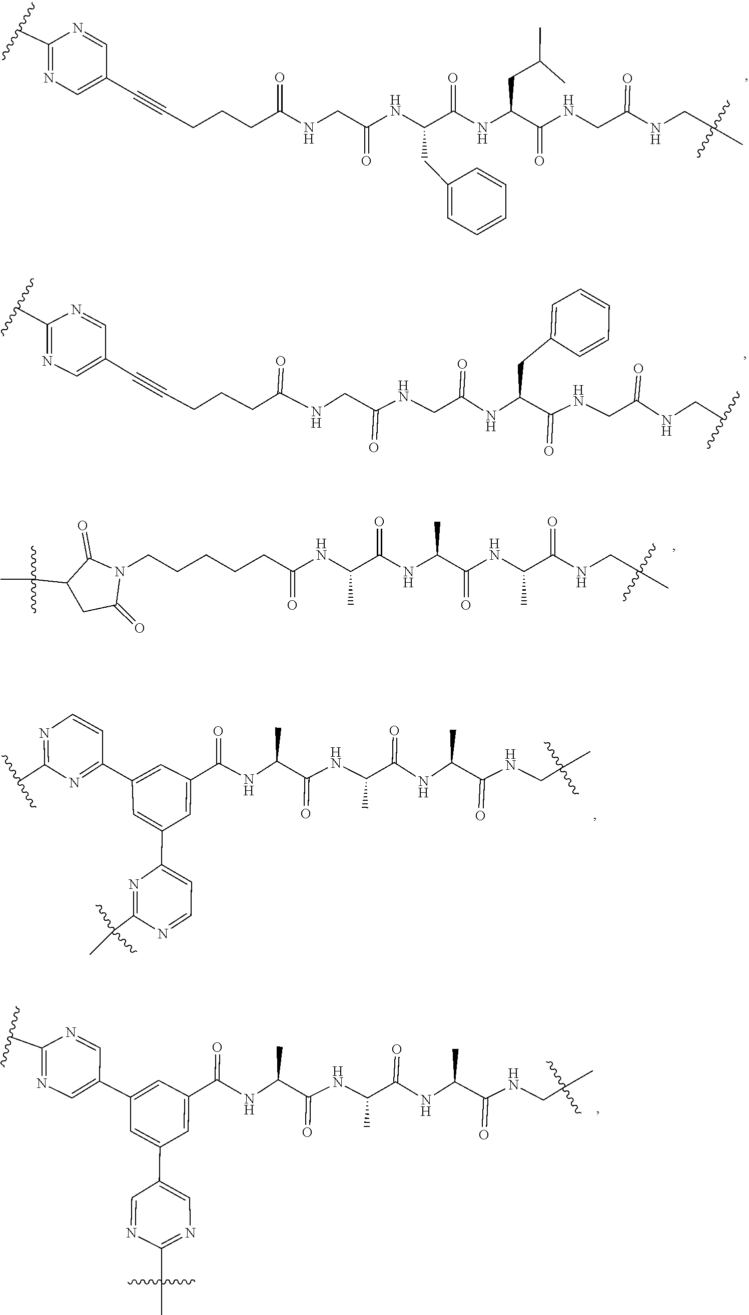 [SMILES: *CNC(=O)CNC(=O)[C@H](CC(C)C)NC(=O)[C@H](Cc1ccccc1)NC(=O)CNC(=O)CCCC#Cc1cnc(*)nc1 ; *CNC(=O)CNC(=O)[C@H](Cc1ccccc1)NC(=O)CNC(=O)CNC(=O)CCCC#Cc1cnc(*)nc1 ; *CNC(=O)[C@H](C)NC(=O)[C@H](C)NC(=O)[C@H](C)NC(=O)CCCCCN1C(=O)CC(*)C1=O ; *CNC(=O)[C@H](C)NC(=O)[C@H](C)NC(=O)[C@H](C)NC(=O)c1cc(-c2ccnc(*)n2)cc(-c2ccnc(*)n2)c1 ; *CNC(=O)[C@H](C)NC(=O)[C@H](C)NC(=O)[C@H](C)NC(=O)c1cc(-c2cnc(*)nc2)cc(-c2cnc(*)nc2)c1]

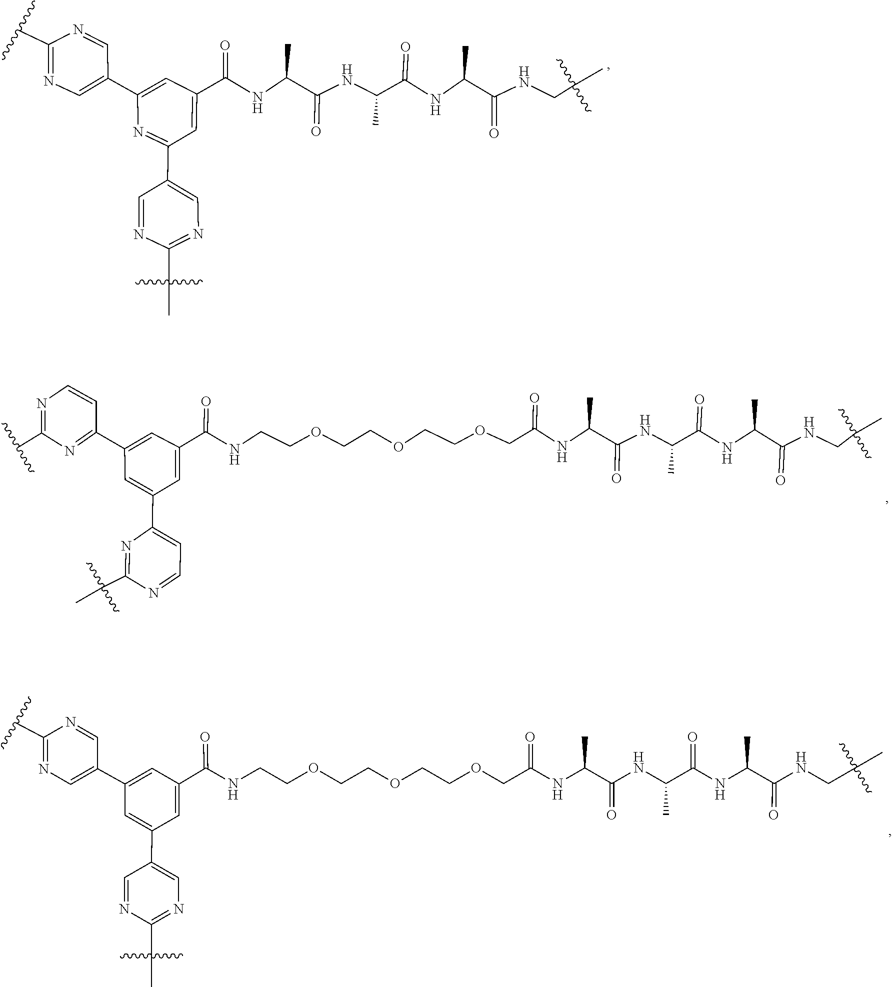 [SMILES: *CNC(=O)[C@H](C)NC(=O)[C@H](C)NC(=O)[C@H](C)NC(=O)c1cc(-c2cnc(*)nc2)nc(-c2cnc(*)nc2)c1 ; *CNC(=O)[C@H](C)NC(=O)[C@H](C)NC(=O)[C@H](C)NC(=O)COCCOCCOCCNC(=O)c1cc(-c2ccnc(*)n2)cc(-c2ccnc(*)n2)c1 ; *CNC(=O)[C@H](C)NC(=O)[C@H](C)NC(=O)[C@H](C)NC(=O)COCCOCCOCCNC(=O)c1cc(-c2cnc(*)nc2)cc(-c2cnc(*)nc2)c1]

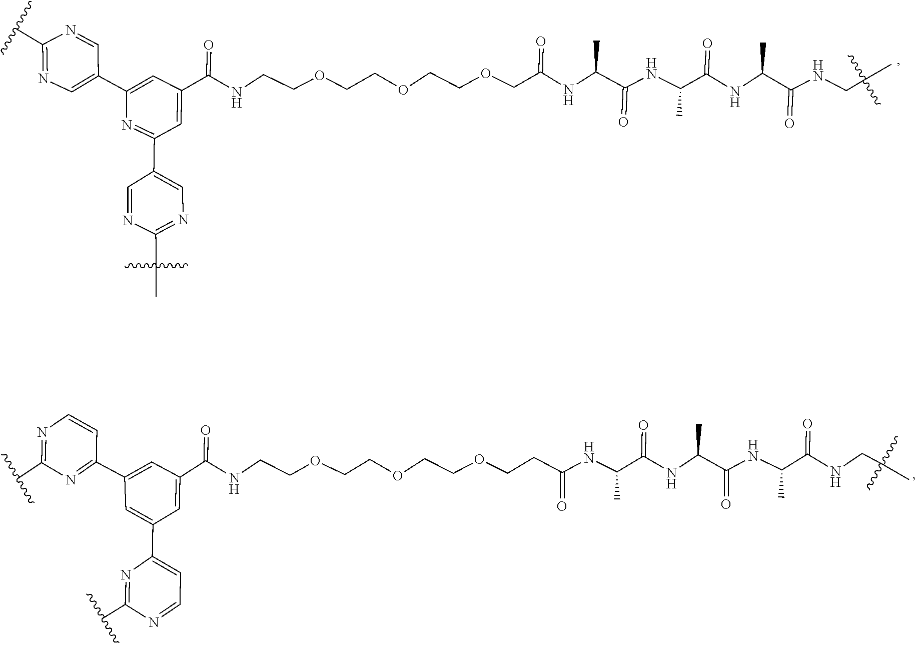 [SMILES: *CNC(=O)[C@H](C)NC(=O)[C@H](C)NC(=O)[C@H](C)NC(=O)COCCOCCOCCNC(=O)c1cc(-c2cnc(*)nc2)nc(-c2cnc(*)nc2)c1 ; *CNC(=O)[C@H](C)NC(=O)[C@H](C)NC(=O)[C@H](C)NC(=O)CCOCCOCCOCCNC(=O)c1cc(-c2ccnc(*)n2)cc(-c2ccnc(*)n2)c1]

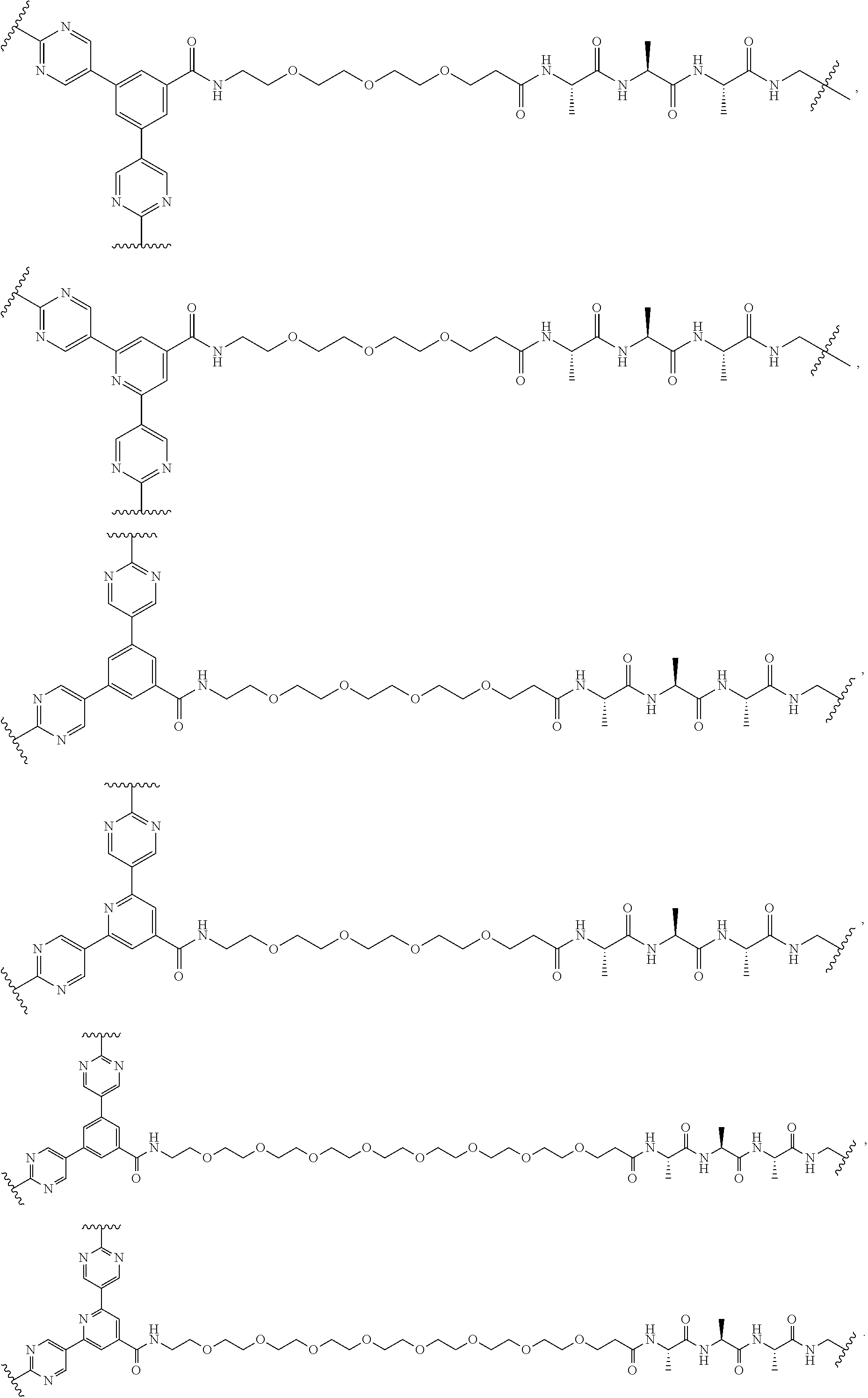

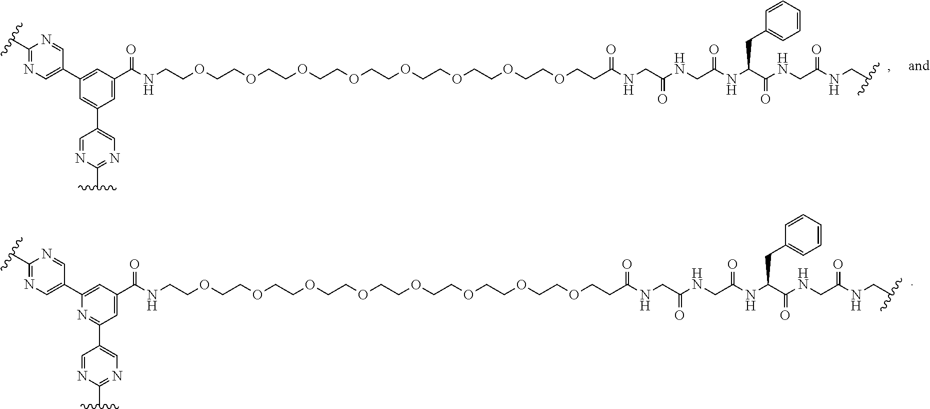 [SMILES: *CNC(=O)CNC(=O)[C@H](Cc1ccccc1)NC(=O)CNC(=O)CNC(=O)CCOCCOCCOCCOCCOCCOCCOCCOCCNC(=O)c1cc(-c2cnc(*)nc2)cc(-c2cnc(*)nc2)c1 ; *CNC(=O)CNC(=O)[C@H](Cc1ccccc1)NC(=O)CNC(=O)CNC(=O)CCOCCOCCOCCOCCOCCOCCOCCOCCNC(=O)c1cc(-c2cnc(*)nc2)nc(-c2cnc(*)nc2)c1]

85. The antibody-drug conjugate of claim 1, wherein 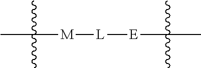
is selected from the following structures:

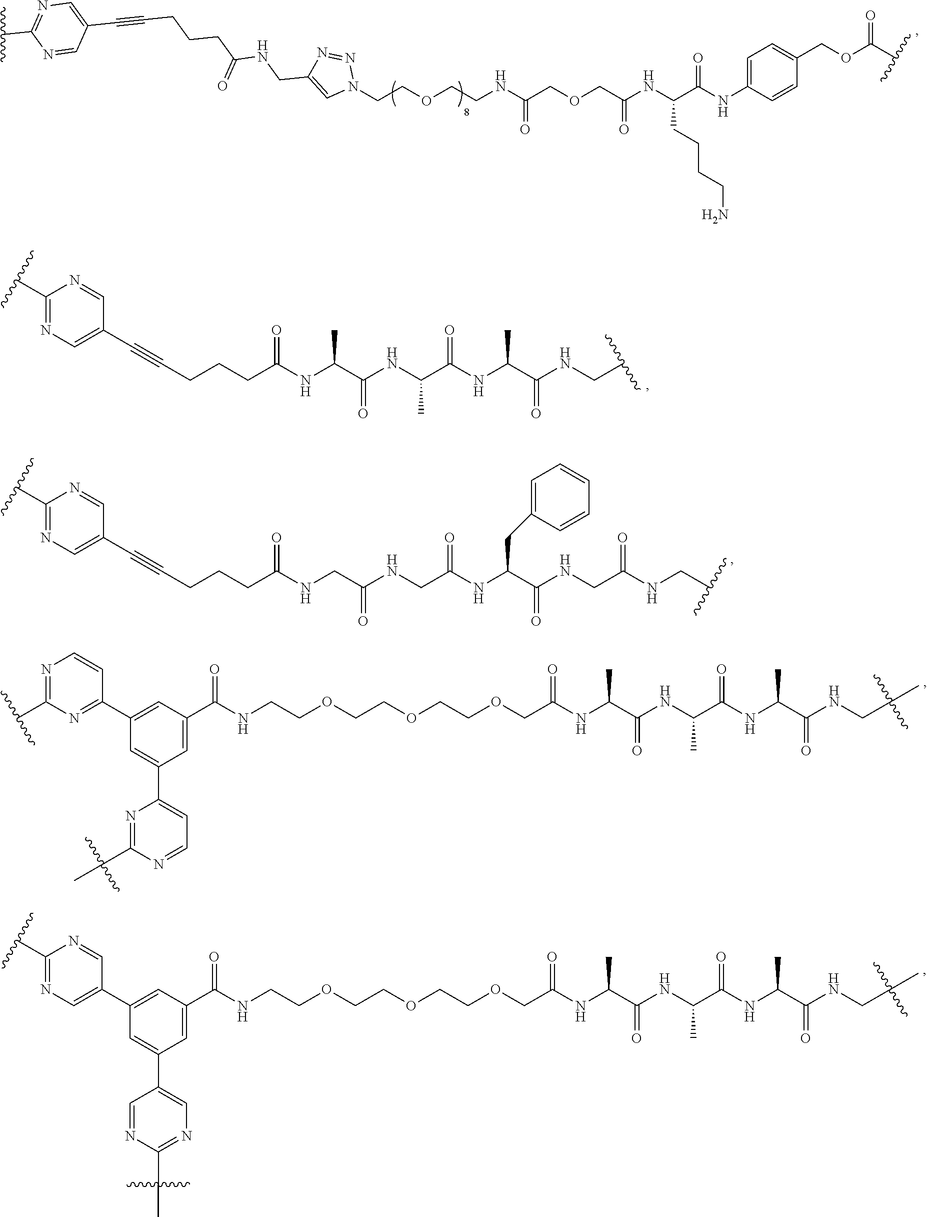

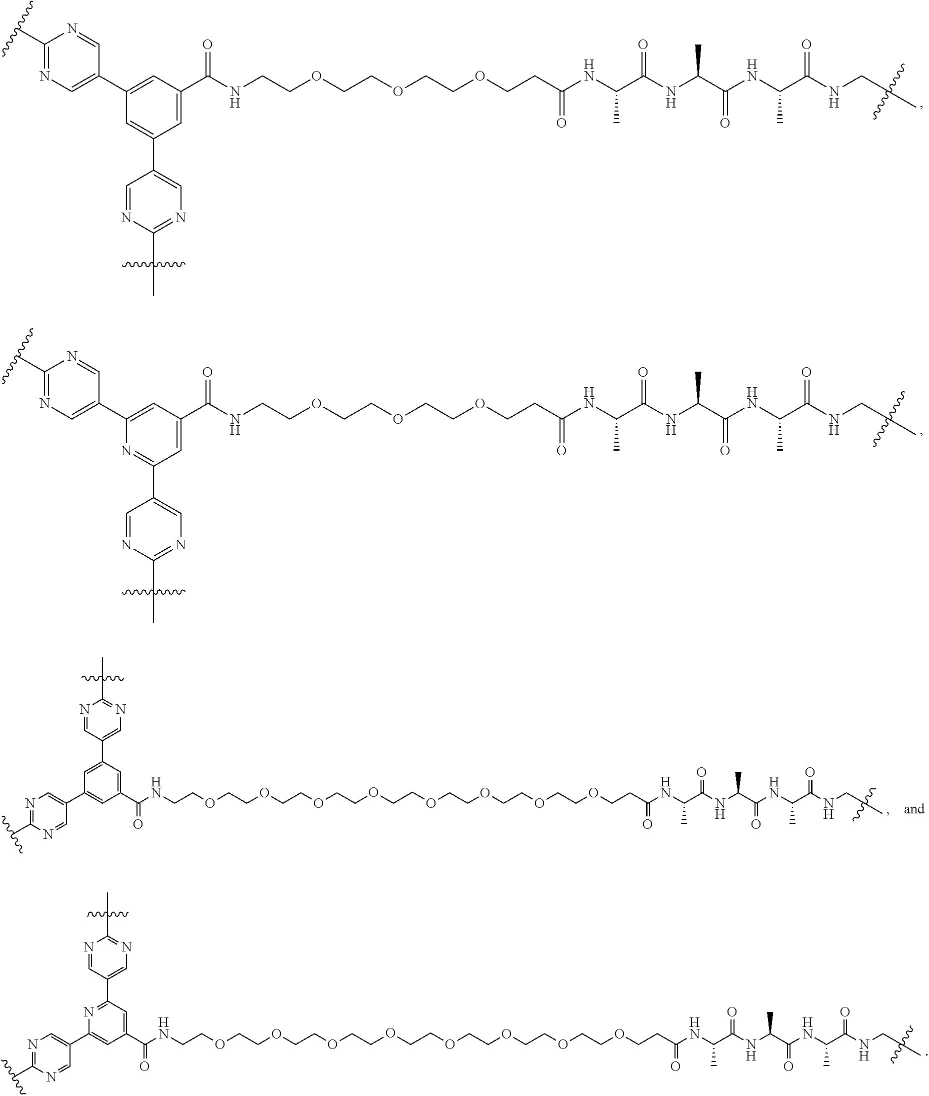 [SMILES: *CNC(=O)[C@H](C)NC(=O)[C@H](C)NC(=O)[C@H](C)NC(=O)CCOCCOCCOCCNC(=O)c1cc(-c2cnc(*)nc2)cc(-c2cnc(*)nc2)c1 ; *CNC(=O)[C@H](C)NC(=O)[C@H](C)NC(=O)[C@H](C)NC(=O)CCOCCOCCOCCNC(=O)c1cc(-c2cnc(*)nc2)nc(-c2cnc(*)nc2)c1 ; *CNC(=O)[C@H](C)NC(=O)[C@H](C)NC(=O)[C@H](C)NC(=O)CCOCCOCCOCCOCCOCCOCCOCCOCCNC(=O)c1cc(-c2cnc(*)nc2)cc(-c2cnc(*)nc2)c1 ; *CNC(=O)[C@H](C)NC(=O)[C@H](C)NC(=O)[C@H](C)NC(=O)CCOCCOCCOCCOCCOCCOCCOCCOCCNC(=O)c1cc(-c2cnc(*)nc2)nc(-c2cnc(*)nc2)c1]

86. The antibody-drug conjugate of claim 1, wherein the cytotoxic drug is selected from the group consisting of tubulin inhibitors, DNA intercalating agents, DNA Topoisomerase inhibitors, and RNA polymerase inhibitors.

87.-91. (canceled)

92. The antibody-drug conjugate of claim 1, wherein the cytotoxic drug is selected from the group consisting of compounds represented by Formula I and Formula II, or pharmaceutically acceptable salts, esters, stereoisomers, tautomers or precursors of the compounds represented by Formula I and Formula II: 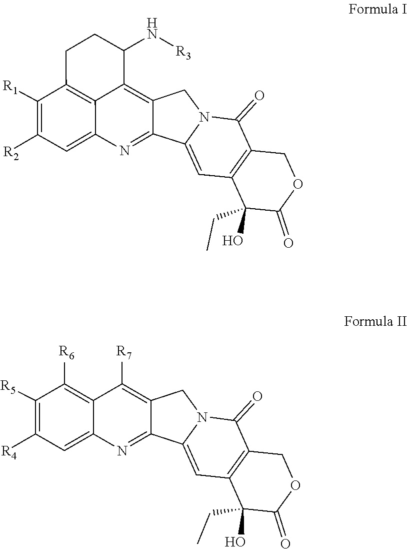
wherein R₁ and R₂ are each independently selected from C₁₋₆ alkyl and halogen;
R₃ is selected from H and —CO—CH₂OH;
R₄ and R₅ are each independently selected from H, halogen, and hydroxyl, or R₄ and R₅ are connected to form a 5-6 membered oxygen-containing heterocyclic ring;
R₆ is selected from hydrogen or —C₁₋₄ alkylene-NRₐR_{b}; and
R₇ is selected from C₁₋₆ alkyl, —C₁₋₄ alkylene-NRₐR_{b}, —C₁₋₄ alkylene-SiRₐR_{b}R_{c}, —SiRₐR_{b}R_{c}, —C₁₋₄ alkylene=N—ORₐ; wherein, Rₐ, R_{b} and R_{c} are independently selected from H, C₁₋₆ alkyl, —SO₂—C₁₋₆ alkyl, and —CO—C₁₋₆ alkyl at each occurrence; wherein optionally Rₐ and R_{b} connected to the associated atoms form a 5-6 membered nitrogen containing heterocyclic ring.

93. The antibody-drug conjugate of claim 1, wherein the cytotoxic drug is selected from the following compounds or pharmaceutically acceptable salts, esters, stereoisomers, tautomers, or prodrugs of said compounds: 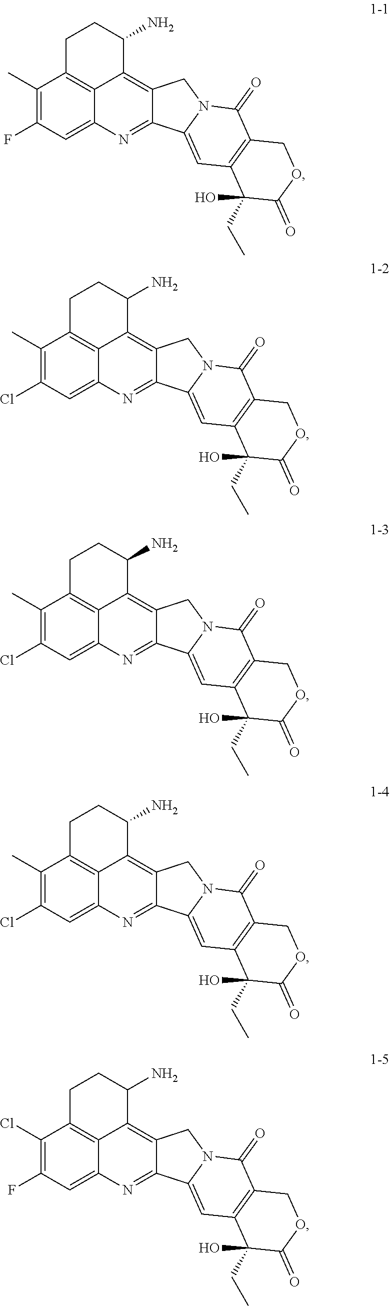 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2C(N)CC3] 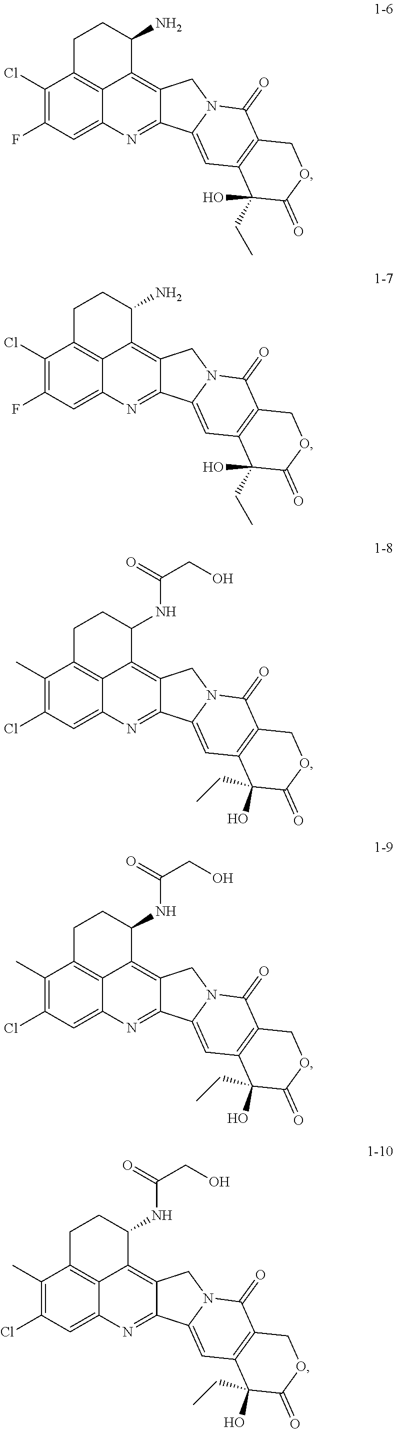 [SMILES: CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3] 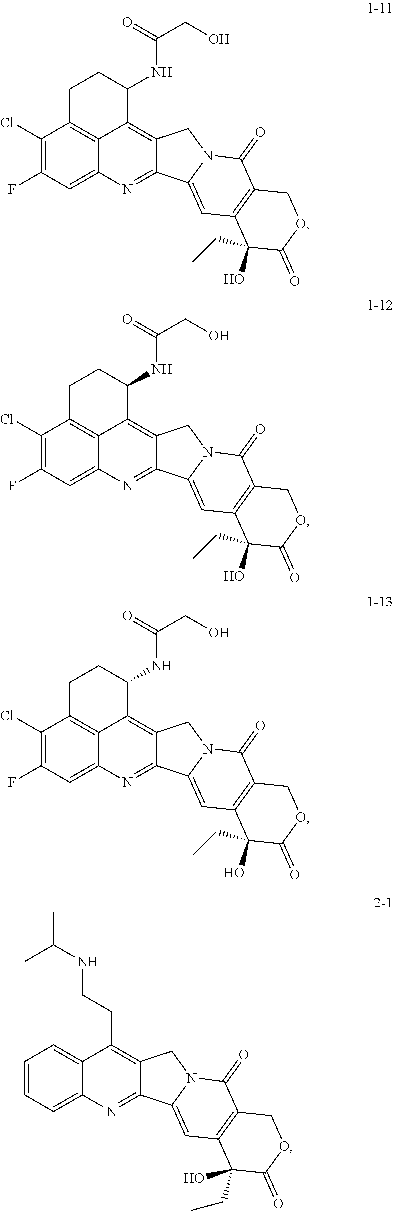 [SMILES: CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1ccccc1c2CCNC(C)C] 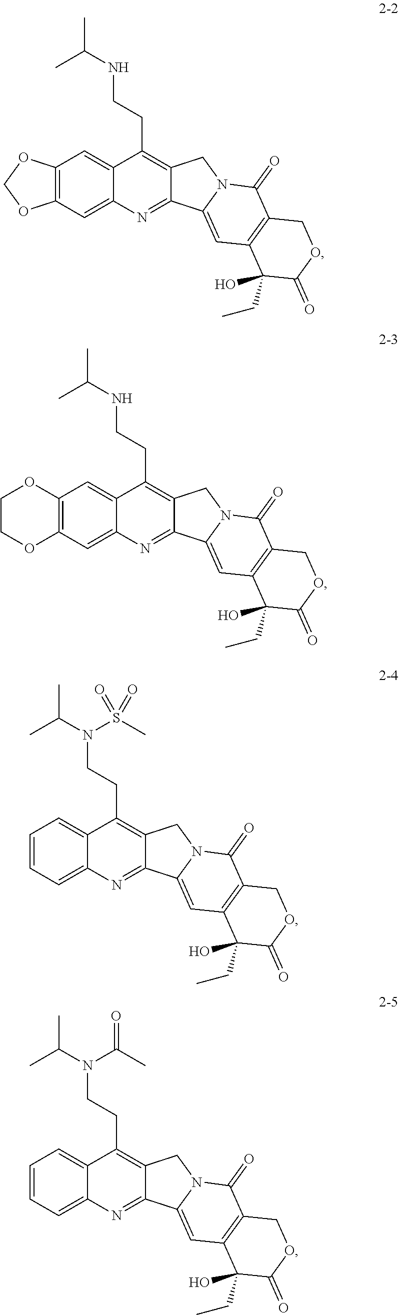 [SMILES: CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1cc3c(cc1c2CCNC(C)C)OCCO3 ; CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1ccccc1c2CCN(C(C)=O)C(C)C] 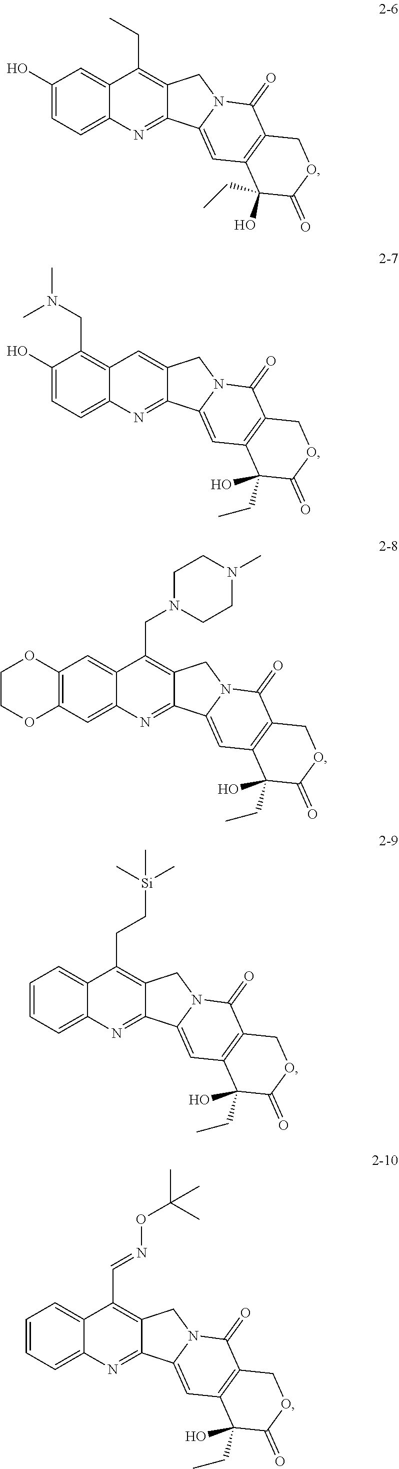 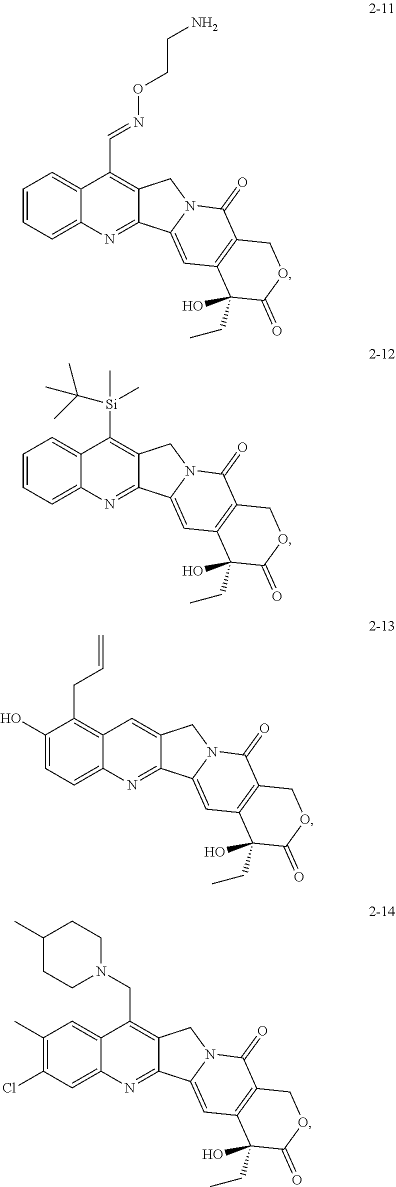 [SMILES: CC[C@@]1(O)C(=O)OCc2c1cc1n(c2=O)Cc2c-1nc1ccccc1c2/C=N/OCCN ; CC[C@@]1(O)C(=O)OCc2c1cc1n(c2=O)Cc2c-1nc1ccccc1c2[Si](C)(C)C(C)(C)C ; C=CCc1c(O)ccc2nc3c(cc12)Cn1c-3cc2c(c1=O)COC(=O)[C@]2(O)CC ; CC[C@@]1(O)C(=O)OCc2c1cc1n(c2=O)Cc2c-1nc1cc(Cl)c(C)cc1c2CN1CCC(C)CC1]
wherein the corresponding residue of the cytotoxic drug obtained after connecting the cytotoxic drug to the linker is D in the formula of claim 1.

94. (canceled)

95. The antibody-drug conjugate of claim 93, wherein D is a monovalent structure obtained by the loss of one H from an —OH, an —NH₂, or a secondary amine on the cytotoxic drug.

96. The antibody-drug conjugate of claim 1, wherein the antibody-drug conjugate is selected from: 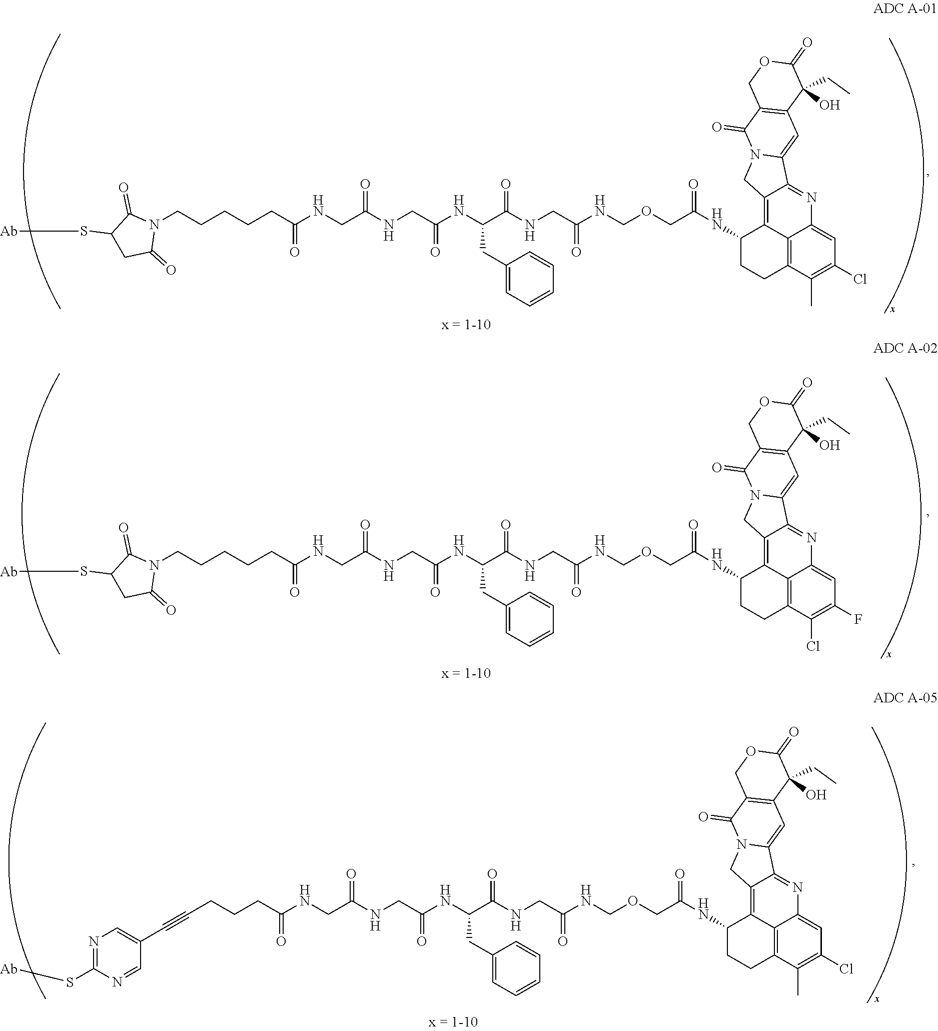 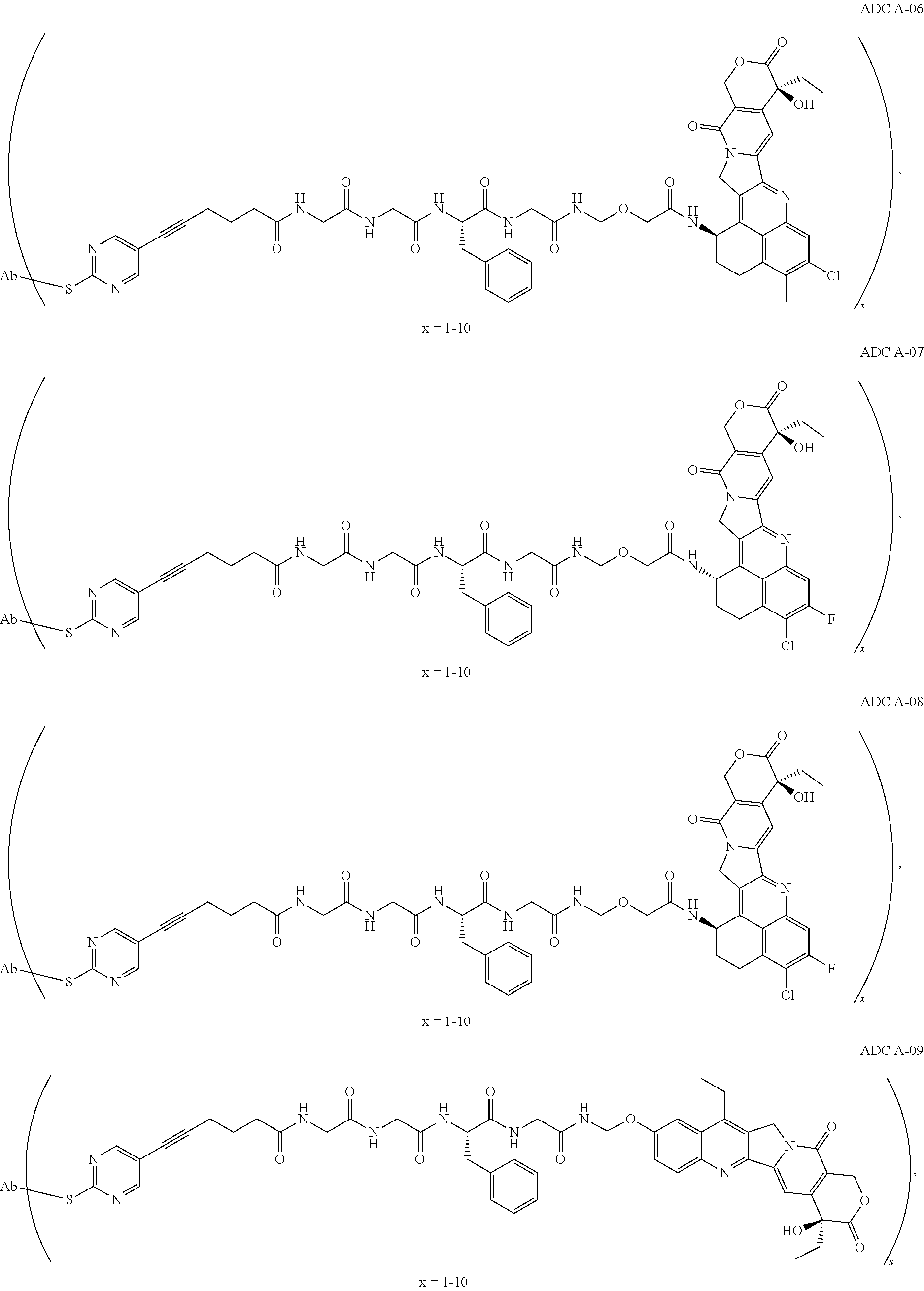 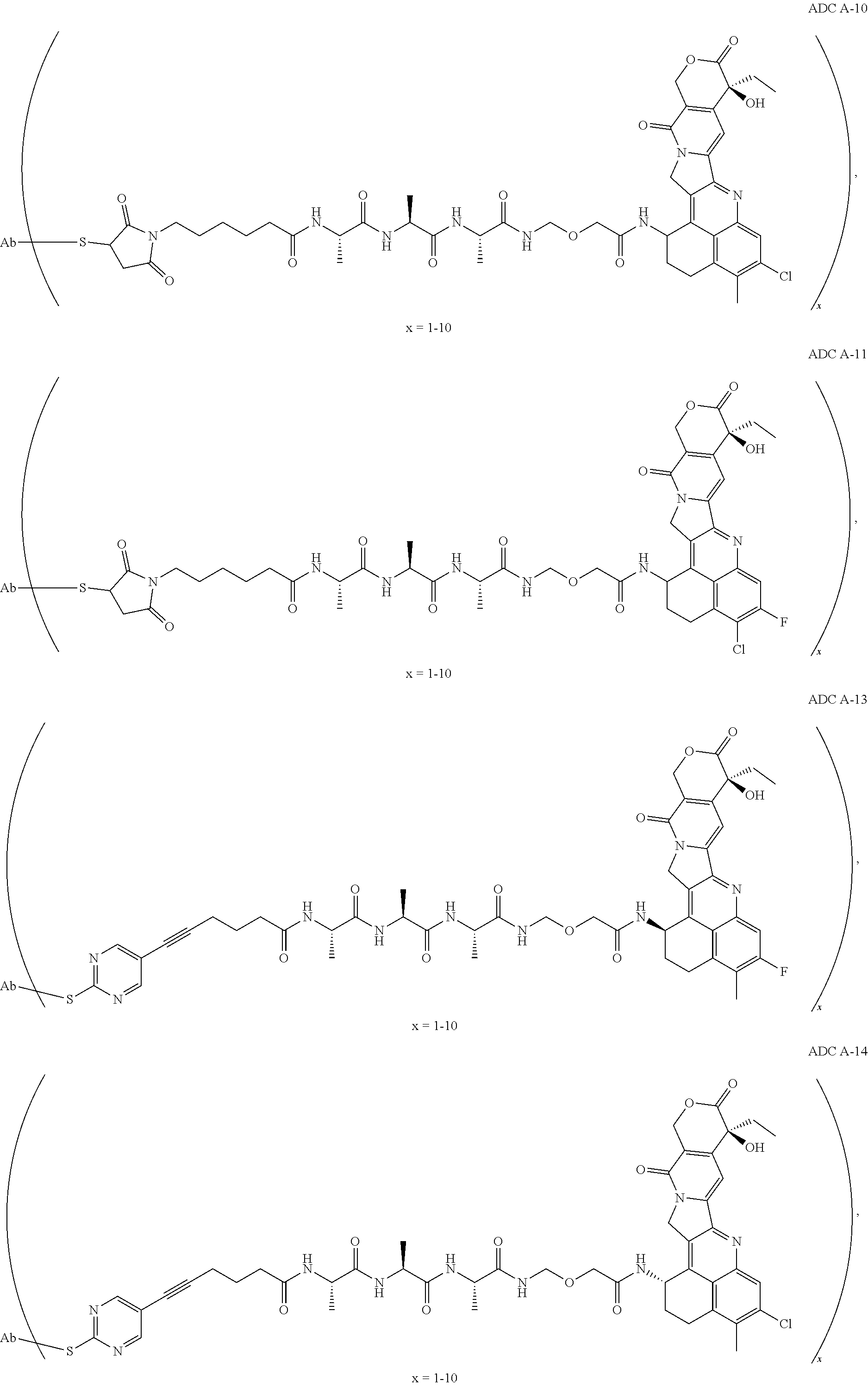 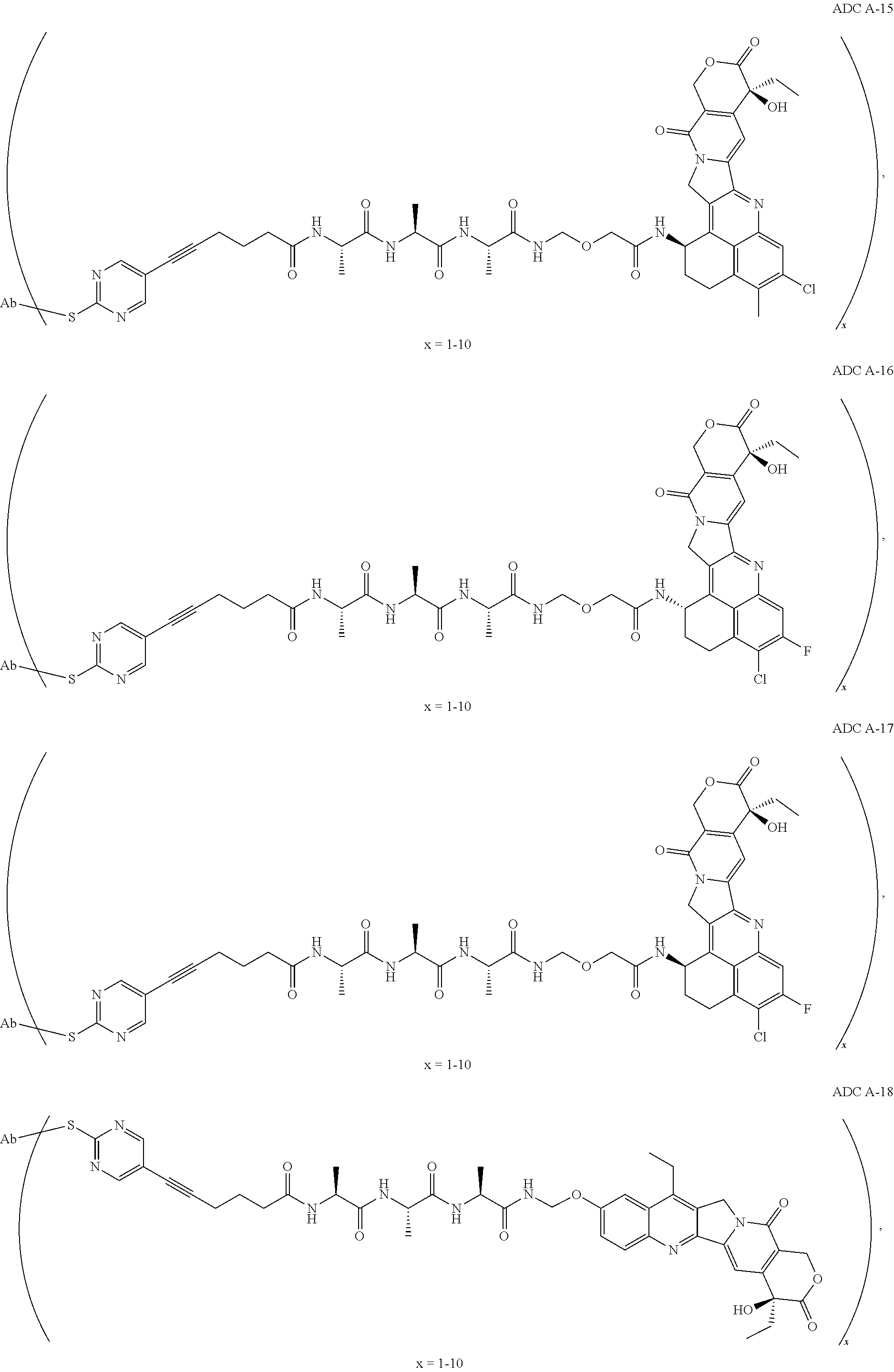 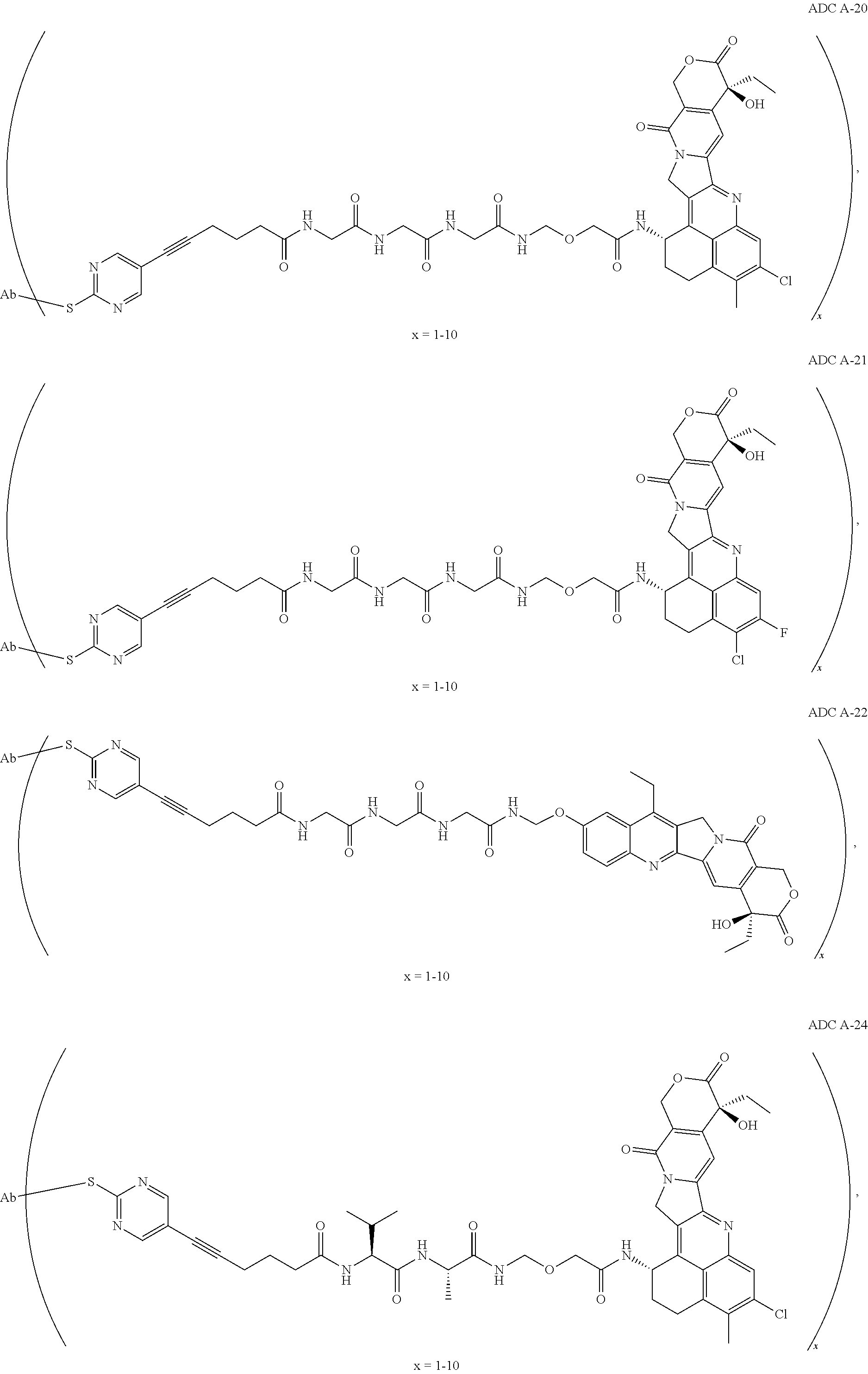 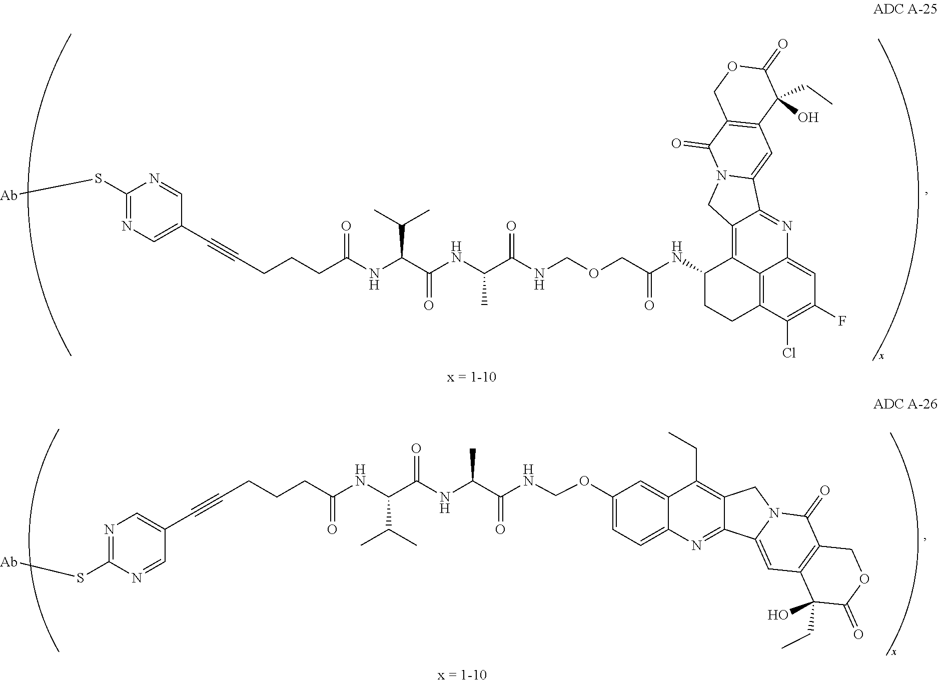 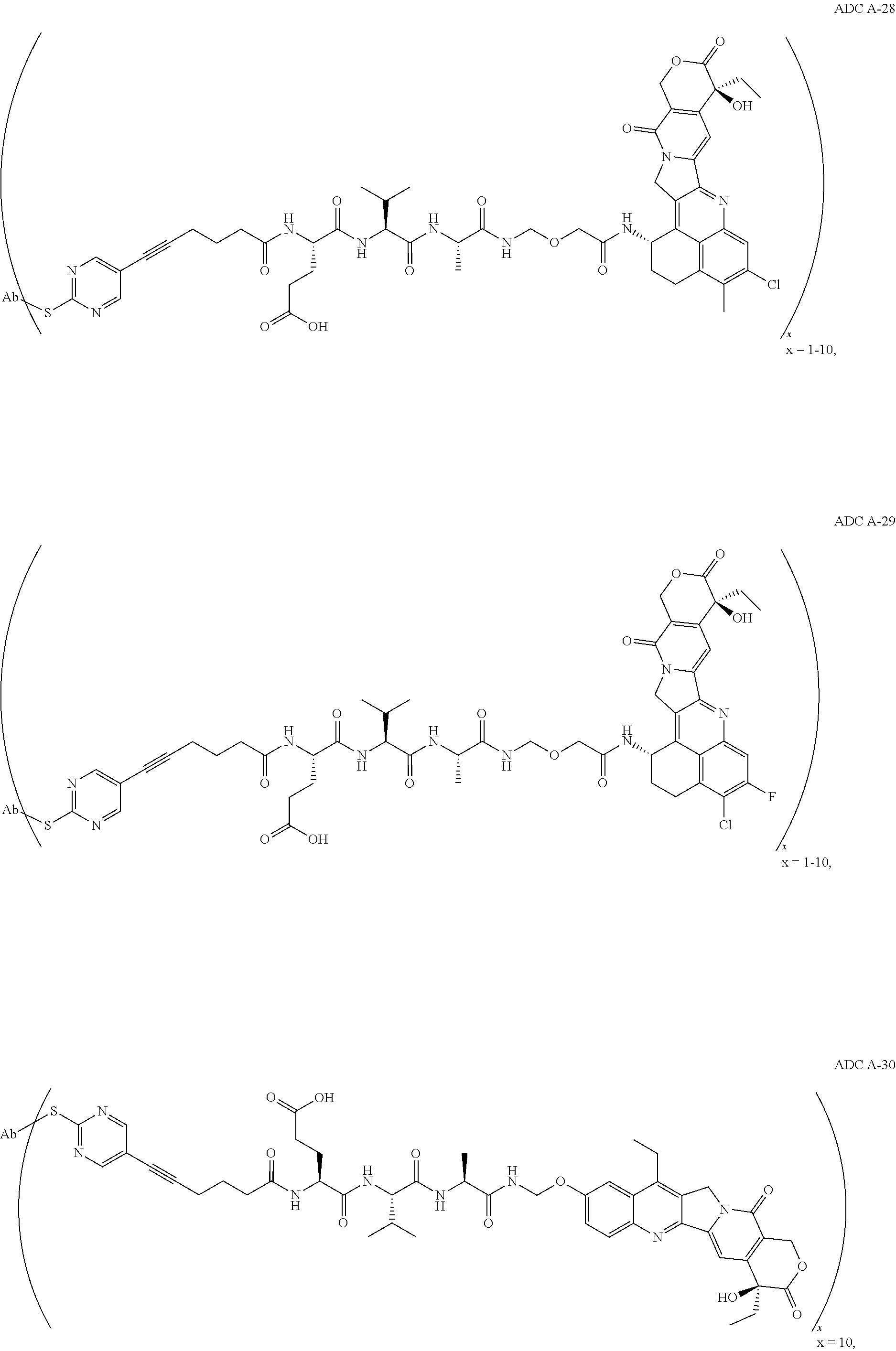 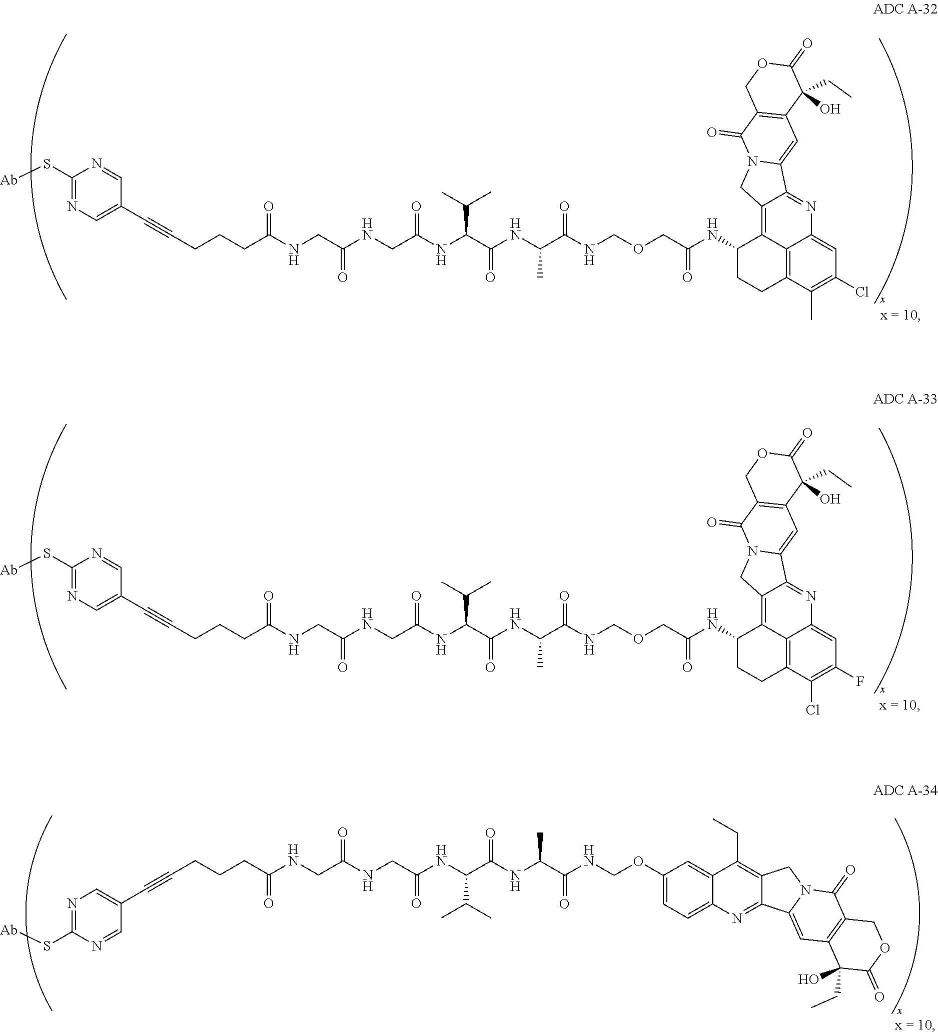 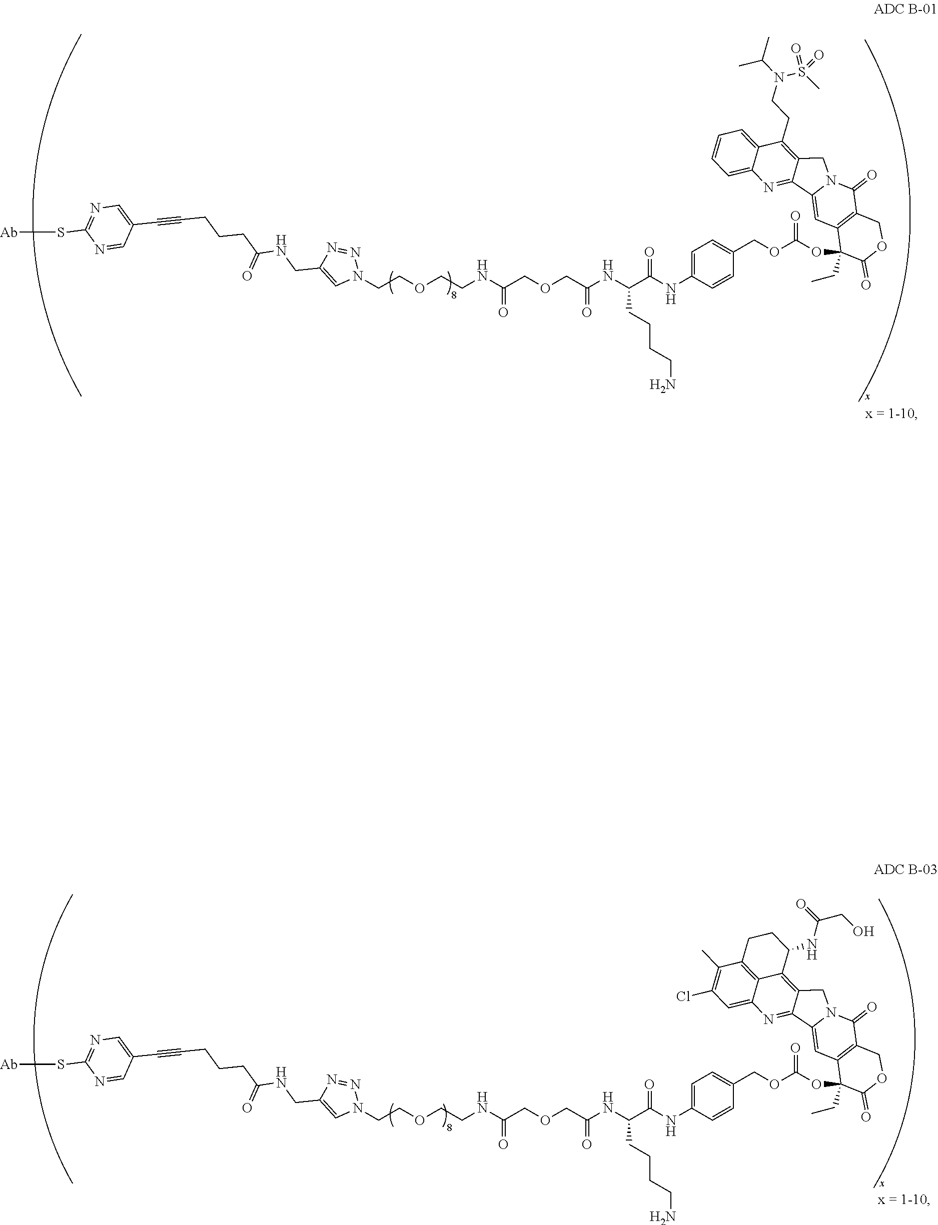 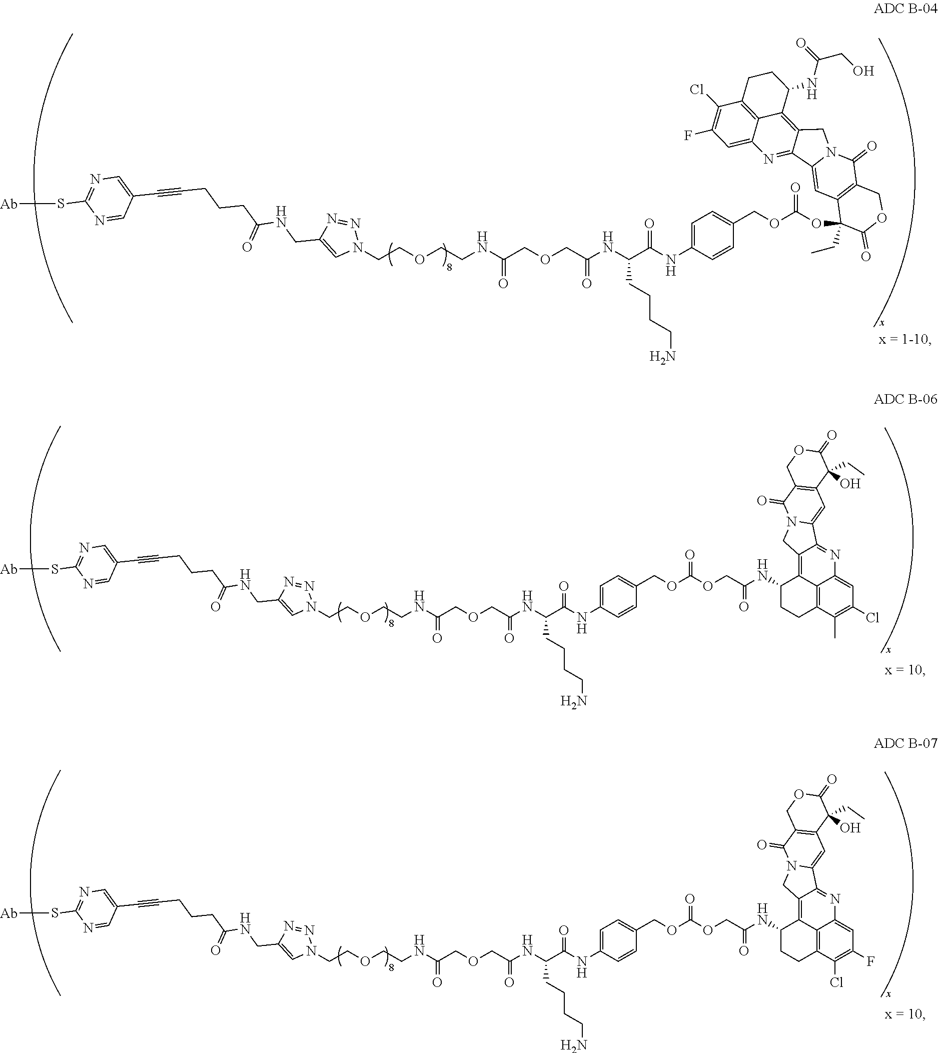 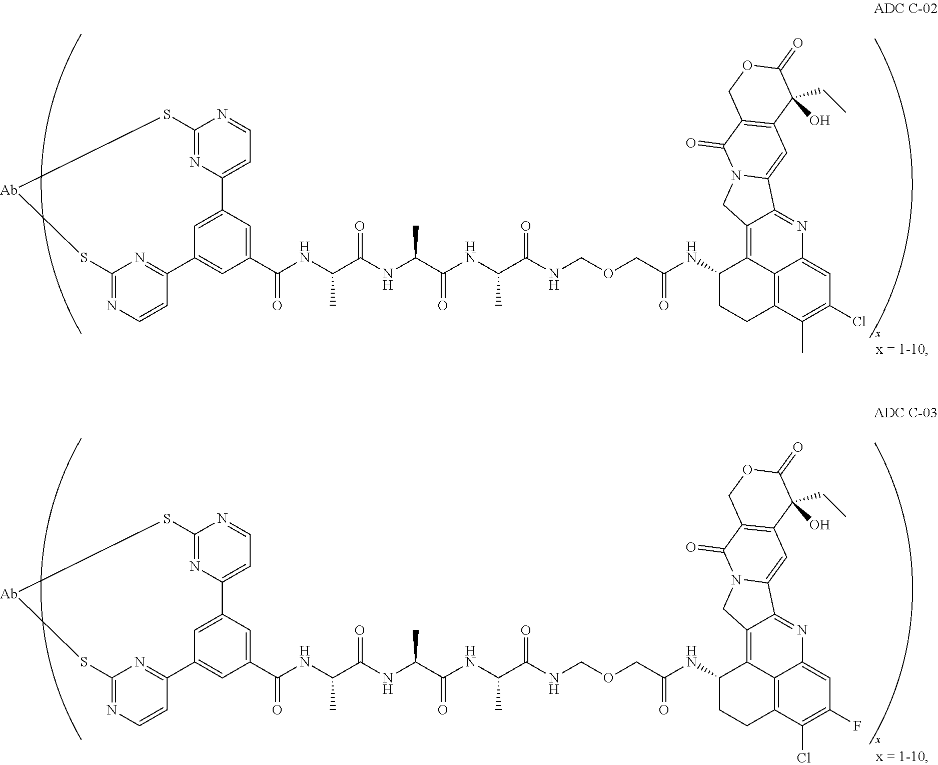 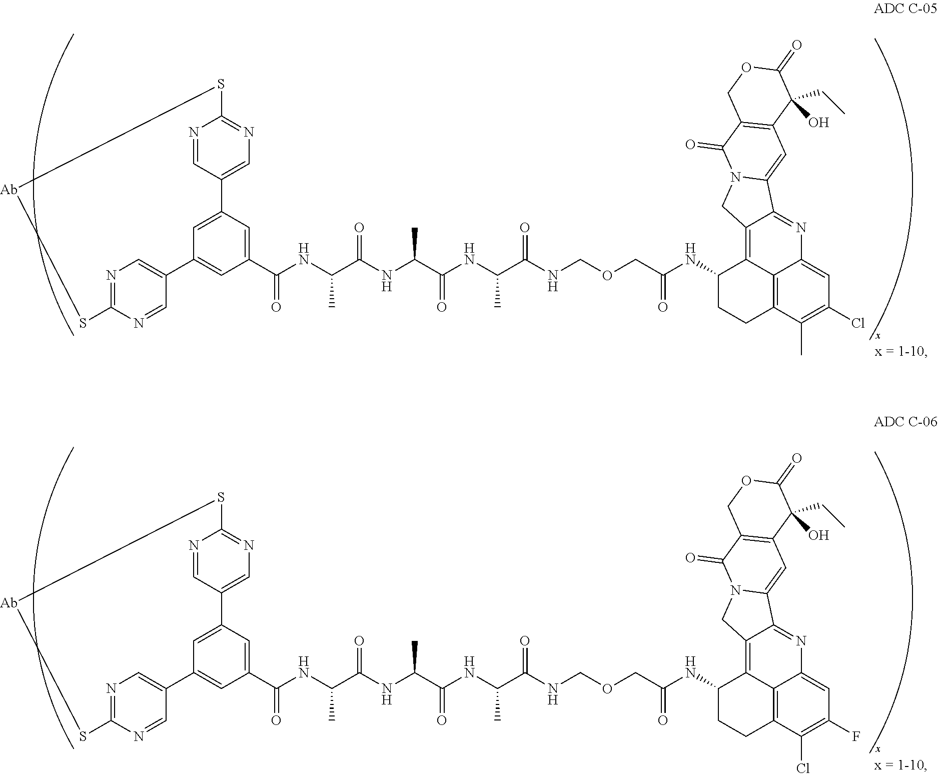 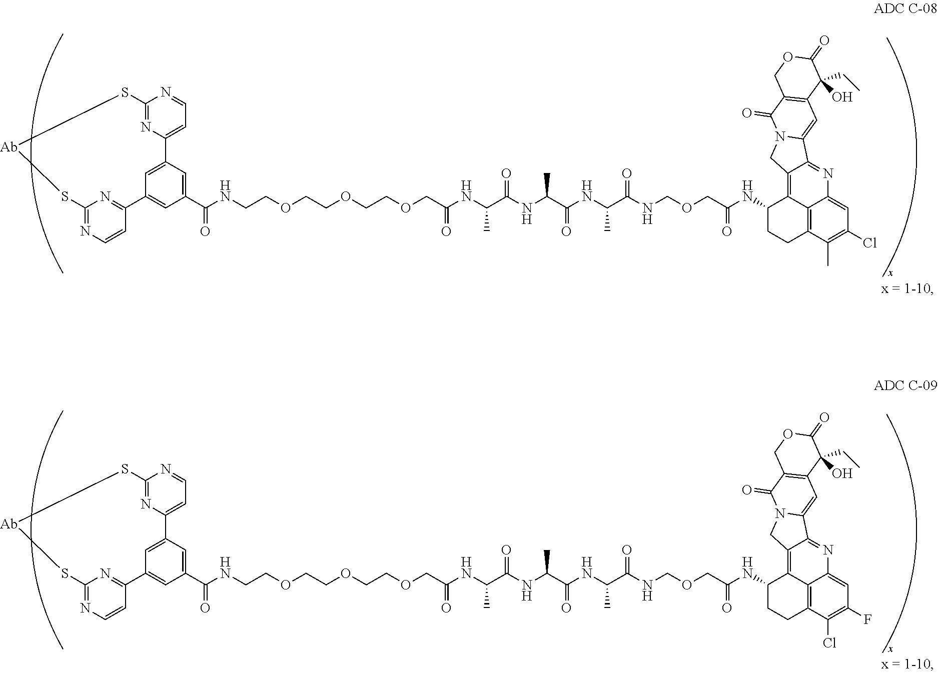 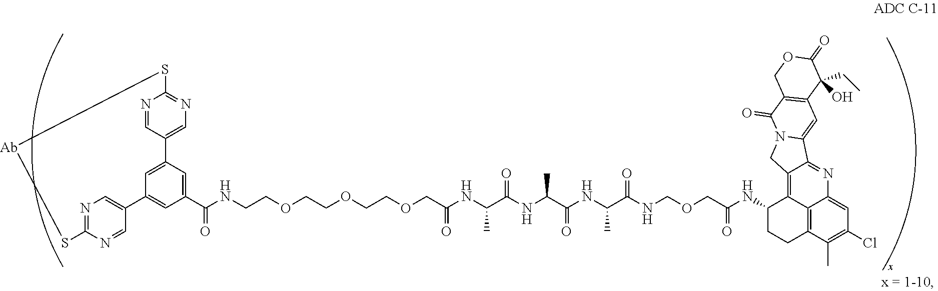 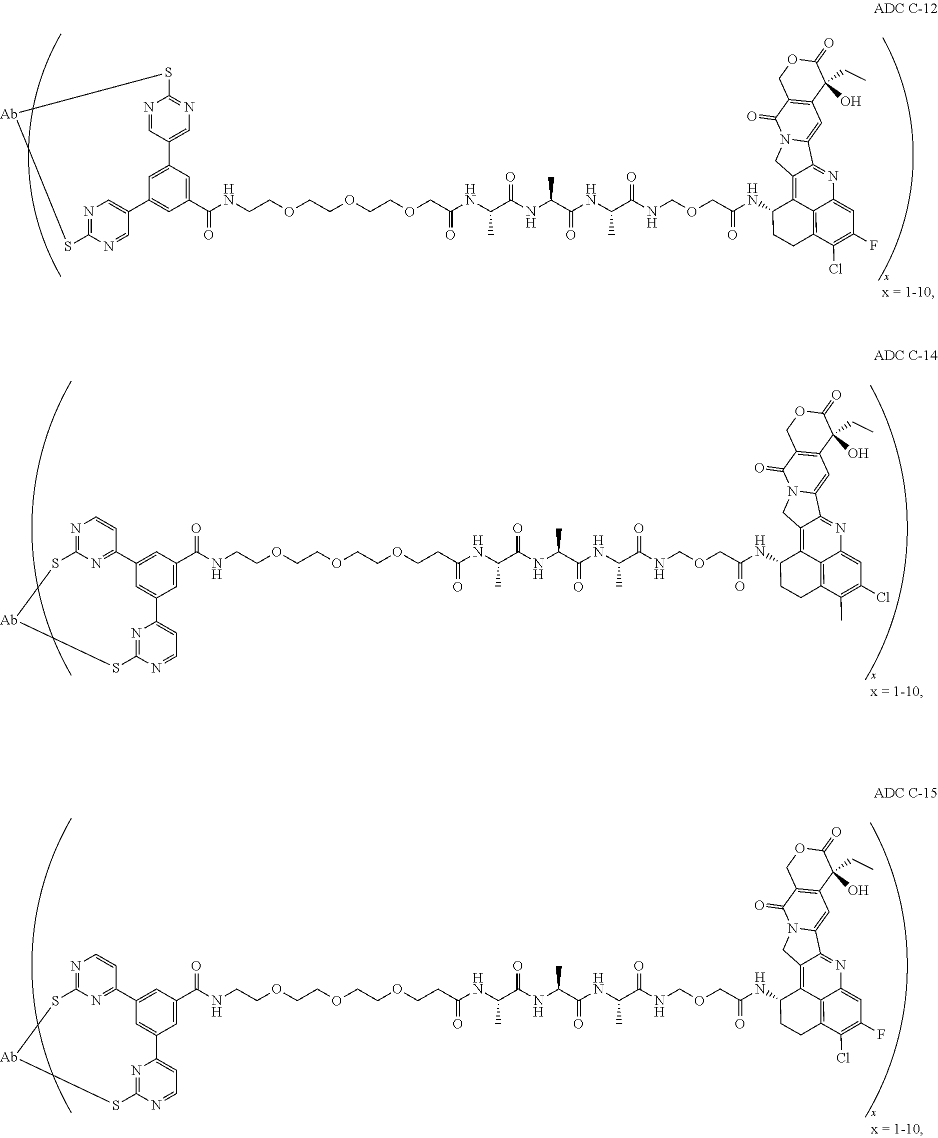 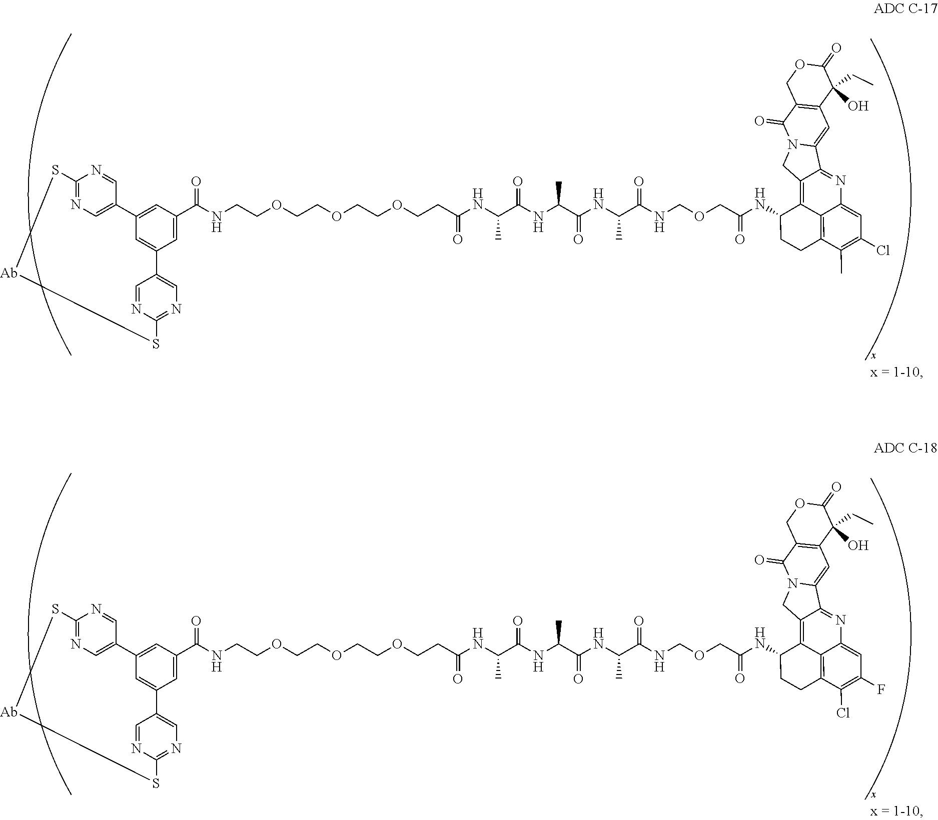 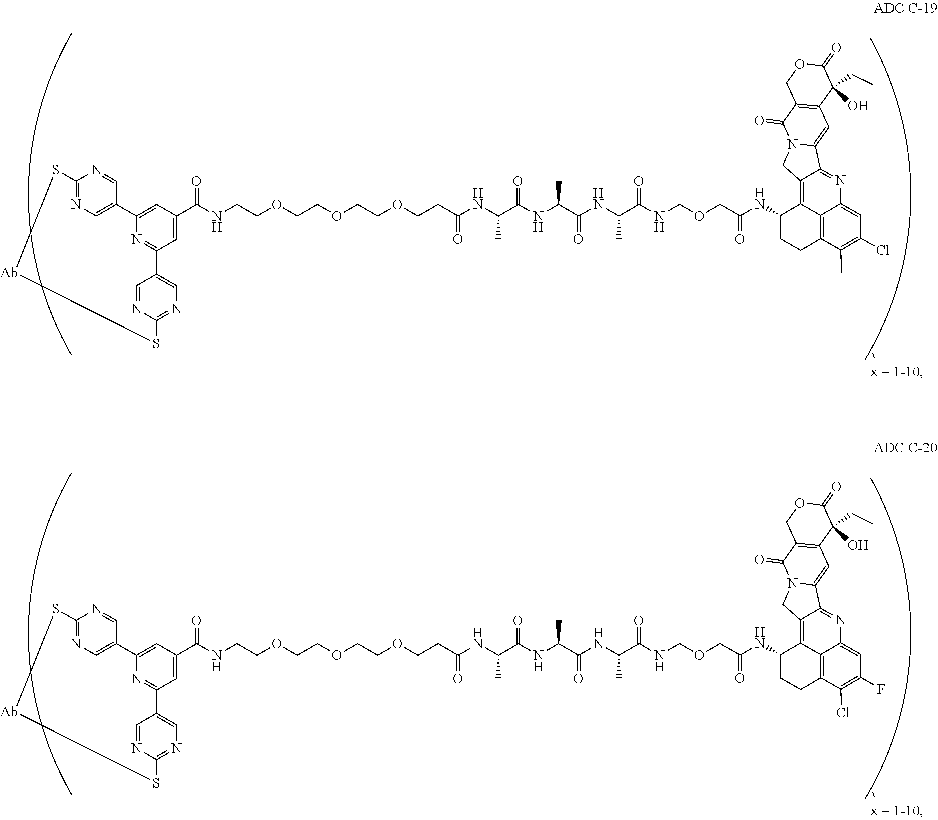 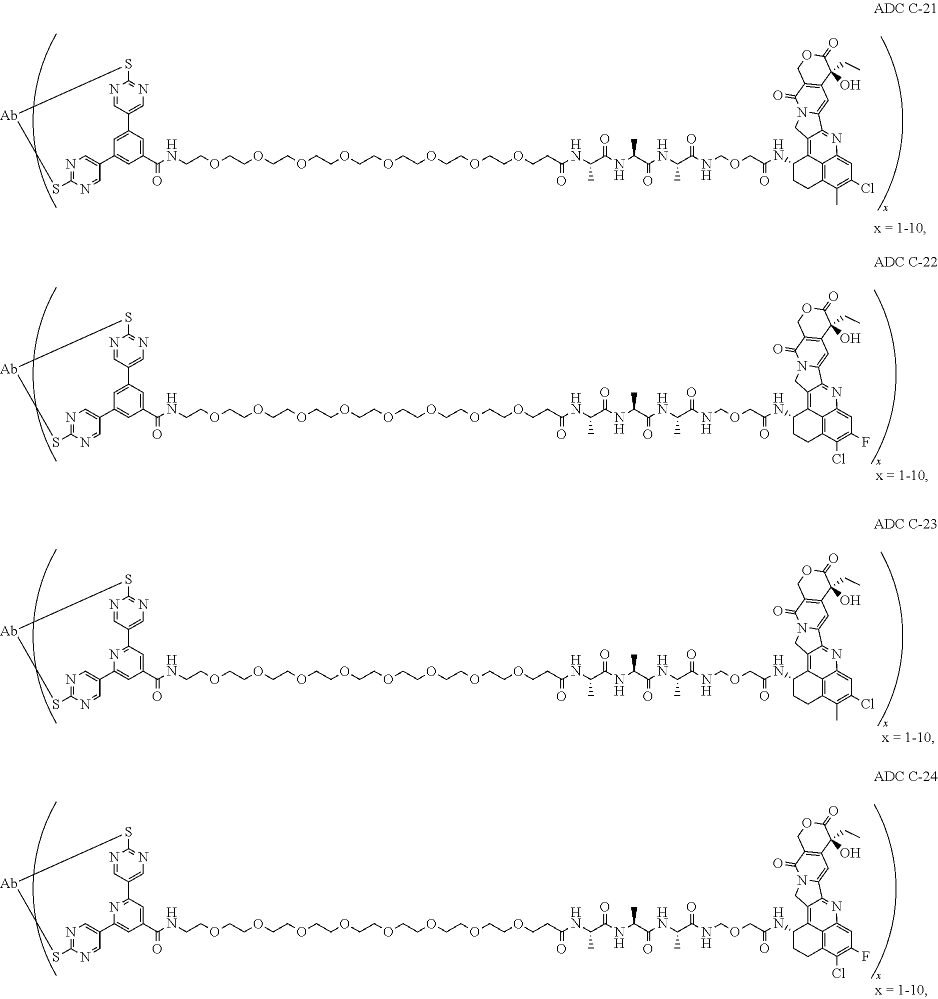 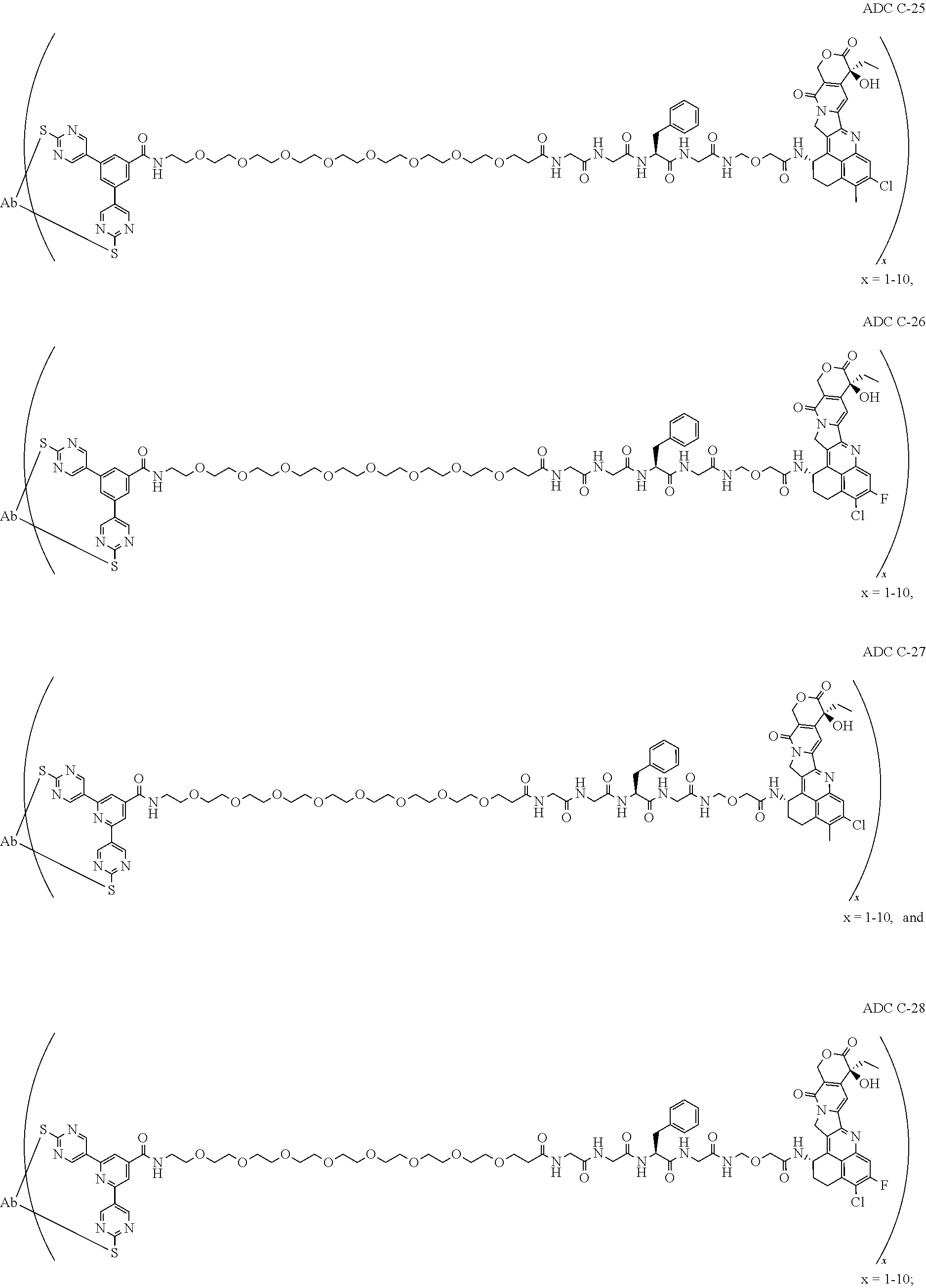
wherein, Ab is the bispecific antibody or an antigen-binding fragment thereof; 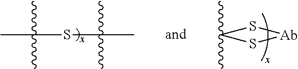
represent the specific connection mode of the sulfhydryl group of a Cys residue in the bispecific antibody or its antigen-binding fragment and M of the antibody-drug conjugate; and x represents the quantity of drug load.

97. The antibody-drug conjugate of claim 96, wherein the sulfhydryl group in the bispecific antibody or its antigen-binding fragment forms a thioether bond with M of the antibody-drug conjugate through an addition reaction or a substitution reaction to obtain the antibody-drug conjugate.

98. The antibody-drug conjugate of claim 96, wherein Ab is a bispecific antibody selected from the group consisting of BsAb 07B, BsAb 10B, BsAb 38B, BsAb 41B, BsAb 49B, BsAb 55B, and BsAb 56B.

99. An antibody-drug conjugate selected from the group consisting of ADC 07B-A-05, ADC 07B-A-14, ADC 38B-A-14, ADC 49B-A-14, ADC 49B-B-01, ADC 49B-A-05, ADC 41B-A-05, ADC 55B-A-14, and ADC 56B-A-14.

100. A composition comprising one or more antibody-drug conjugates of claim 1.

101. (canceled)

102. The composition of claim 100, wherein the composition has a DAR value of 1, 2, 3, 4, 5, 6, 7, 8, 9, 10, 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10.

103.-105. (canceled)

106. A pharmaceutical composition comprising one or more antibody-drug conjugates of claim 1, and one or more pharmaceutically acceptable carriers and/or excipients.

107.-111. (canceled)

112. A method for inhibiting the activity of c-MET and/or EGFR in cells, the method comprising contacting the cells with the antibody-drug conjugate of claim 1, wherein the cells are cells that express c-MET and/or EGFR.

113. A method for preventing, treating, and/or acting as an adjuvant in treating a disease associated with c-MET and/or EGFR in a subject in need thereof, the method comprising administering to the subject an effective amount of the antibody-drug conjugate of claim 1, wherein the disease associated with c-MET and/or EGFR is a disease comprising EGFR activating mutations, EGFR gene amplification, elevated circulating HGF levels, c-MET activating mutations, and/or c-MET gene amplification, optionally wherein the disease associated with c-MET and/or EGFR is cancer.

114. The method of claim 113, wherein the cancer is selected from the group consisting of epithelial cell carcinoma, breast cancer, ovarian cancer, lung adenocarcinoma, small cell lung cancer, non-small cell lung cancer, oral cancer, colorectal cancer, anal cancer, and prostate cancer, bladder cancer, pharyngeal cancer, nasal cancer, pancreatic cancer, skin cancer, tongue cancer, esophageal cancer, vaginal cancer, cervical cancer, spleen cancer, testicular cancer, gastric cancer, thymus cancer, thyroid cancer, hepatocellular carcinoma, sporadic or hereditary papillary renal cell carcinoma, colon cancer, liver cancer, kidney cancer, or head and neck cancer

115.-118. (canceled)


SEQUENCE LISTING (75 sequences)

SEQ ID NO: 1 - protein, 220 aa, synthetic construct
  source 1..220: note = LC for BsAb 07B, 49B, and 55B [anti-c-MET-VL:CL]
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, synthetic construct
  source 1..449: note = HC for BsAb 07B [anti-c-MET-VH: Fc:D356E:L358M:KIH-knob:S354C:T366W (Fc Eu numbering scheme)]
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 478 aa, synthetic construct
  source 1..478: note = scFv-Fc for BsAb 07B [anti-EGFR-VL:anti-EGFR-VH: Fc:D356E:L358M:KIH-hole:Y349C: T366S:L368A:Y407V (Fc Eu numbering scheme)]
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 4 - protein, 214 aa, synthetic construct
  source 1..214: note = LC for BsAb 10B, 38B, and 41B [anti-EGFR-VL:CL]
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 455 aa, synthetic construct
  source 1..455: note = HC for BsAb10B [anti-EGFR-VH: Fc:D356E:L358M:KIH-knob:S354C:T366W (Fc Eu numbering scheme)]
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 478 aa, synthetic construct
  source 1..478: note = scFv-Fc for BsAb 10B [anti-c-MET-VL:anti-c-MET-VH: Fc:D356E:L358M:KIH-hole:Y349C: T366S:L368A:Y407V (Fc Eu numbering scheme)]
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKGGGGSGGGGSGGGGSEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 7 - protein, 455 aa, synthetic construct
  source 1..455: note = HC for BsAb 38B and 41B [anti-EGFR-VH: Fc:L234A:L235A:P329A: KIH-knob:S354C:T366W (Fc Eu numbering scheme)]
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 478 aa, synthetic construct
  source 1..478: note = scFv-Fc for BsAb 38B and 56B [anti-c-MET-VL:anti-c-MET-VH: Fc:L234A:L235A:P329A: KIH-hole:Y349C: T366S:L368A:Y407V (Fc Eu numbering scheme)]
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKGGGGSGGGGSGGGGSEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 9 - protein, 449 aa, synthetic construct
  source 1..449: note = HC for BsAb 49B and 55B [anti-c-MET-VH: Fc:L234A:L235A:P329A: KIH-knob:S354C:T366W (Fc Eu numbering scheme)]
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 478 aa, synthetic construct
  source 1..478: note = scFv-Fc for BsAb 49B [anti-EGFR-VL:anti-EGFR-VH: Fc:L234A:L235A:P329A: KIH-hole:Y349C: T366S:L368A:Y407V (Fc Eu numbering scheme)]
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 709 aa, synthetic construct
  source 1..709: note = scFab-Fc for BsAb 41B [anti-cMET-VL (Q106C):anti-c-MET-VH (G304C): Fc:L234A:L235A:P329A: KIH-hole:Y349C: T366S:L368A:Y407V (Fc Eu numbering scheme)]
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGCGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSEVQLVESGGGLVQPGGSLRLSCAASGYTFTSYWLHWVRQAPGKCLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 12 - protein, 212 aa, synthetic construct
  source 1..212: note = VL-CH1 for BsAb 55B [anti-EGFR-VL:CH1]
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 13 - protein, 459 aa, synthetic construct
  source 1..459: note = VH-CL-Fc for BsAb 55B [anti-EGFR-VH:CL: Fc:L234A:L235A:P329A: KIH-hole:Y349C: T366S:L368A:Y407V (Fc Eu numbering scheme)]
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 14 - protein, 478 aa, synthetic construct
  source 1..478: note = scFv-Fc for BsAb 56B [anti-EGFR-VL:anti-EGFR-VH: Fc:L234A:L235A:P329A: KIH-knob:S354C:T366W (Fc Eu numbering scheme)]
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 107 aa, synthetic construct [sequence omitted]

SEQ ID NO: 16 - protein, 125 aa, synthetic construct [sequence omitted]

SEQ ID NO: 17 - protein, 113 aa, synthetic construct [sequence omitted]

SEQ ID NO: 18 - protein, 119 aa, synthetic construct [sequence omitted]

SEQ ID NO: 19 - protein, 11 aa, synthetic construct
  source 1..11: note = Anti-EGFR-CDR-L1 (Kabat/Chothia/Abm definition)
RASQDISSALV

SEQ ID NO: 20 - protein, 6 aa, synthetic construct
  source 1..6: note = Anti-EGFR-CDR-L1 (IMGT definition)
QDISSA

SEQ ID NO: 21 - protein, 7 aa, synthetic construct
  source 1..7: note = Anti-EGFR-CDR-L2 (Kabat/Chothia/Abm definition)
DASSLES

SEQ ID NO: 22 - skipped

SEQ ID NO: 23 - protein, 9 aa, synthetic construct
  source 1..9: note = Anti-EGFR-CDR-L3 (Kabat/Chothia/Abm/IMGT definition)
QQFNSYPLT

SEQ ID NO: 24 - protein, 5 aa, synthetic construct
  source 1..5: note = Anti-EGFR-CDR-H1 (Kabat definition)
TYGMH

SEQ ID NO: 25 - protein, 7 aa, synthetic construct
  source 1..7: note = Anti-EGFR-CDR-H1 (Chothia definition)
GFTFSTY

SEQ ID NO: 26 - protein, 8 aa, synthetic construct
  source 1..8: note = Anti-EGFR-CDR-H1 (IMGT definition)
GFTFSTYG

SEQ ID NO: 27 - protein, 10 aa, synthetic construct
  source 1..10: note = Anti-EGFR-CDR-H1 (Abm definition)
GFTFSTYGMH

SEQ ID NO: 28 - protein, 17 aa, synthetic construct
  source 1..17: note = Anti-EGFR-CDR-H2 (Kabat definition)
VIWDDGSYKYYGDSVKG

SEQ ID NO: 29 - protein, 6 aa, synthetic construct
  source 1..6: note = Anti-EGFR-CDR-H2 (Chothia definition)
WDDGSY

SEQ ID NO: 30 - protein, 8 aa, synthetic construct
  source 1..8: note = Anti-EGFR-CDR-H2 (IMGT definition)
IWDDGSYK

SEQ ID NO: 31 - protein, 10 aa, synthetic construct
  source 1..10: note = Anti-EGFR-CDR-H2 (Abm definition)
VIWDDGSYKY

SEQ ID NO: 32 - protein, 16 aa, synthetic construct
  source 1..16: note = Anti-EGFR-CDR-H3 (Kabat/Chothia/Abm definition)
DGITMVRGVMKDYFDY

SEQ ID NO: 33 - protein, 18 aa, synthetic construct
  source 1..18: note = Anti-EGFR-CDR-H3 (IMGT definition)
ARDGITMVRGVMKDYFDY

SEQ ID NO: 34 - protein, 17 aa, synthetic construct
  source 1..17: note = Anti-c-MET-CDR-L1 (Kabat/Chothia/Abm definition)
KSSQSLLYTSSQKNYLA

SEQ ID NO: 35 - protein, 12 aa, synthetic construct
  source 1..12: note = Anti-c-MET-CDR-L1 (IMGT definition)
QSLLYTSSQKNY

SEQ ID NO: 36 - protein, 7 aa, synthetic construct
  source 1..7: note = Anti-c-MET-CDR-L2 (Kabat/Chothia/Abm definition)
WASTRES

SEQ ID NO: 37 - skipped

SEQ ID NO: 38 - protein, 9 aa, synthetic construct
  source 1..9: note = Anti-c-MET-CDR-L3 (Kabat/Chothia/Abm/IMGT definition)
QQYYAYPWT

SEQ ID NO: 39 - protein, 5 aa, synthetic construct
  source 1..5: note = Anti-c-MET-CDR-H1 (Kabat definition)
SYWLH

SEQ ID NO: 40 - protein, 7 aa, synthetic construct
  source 1..7: note = Anti-c-MET-CDR-H1 (Chothia definition)
GYTFTSY

SEQ ID NO: 41 - protein, 8 aa, synthetic construct
  source 1..8: note = Anti-c-MET-CDR-H1 (IMGT definition)
GYTFTSYW

SEQ ID NO: 42 - protein, 10 aa, synthetic construct
  source 1..10: note = Anti-c-MET-CDR-H1 (Abm definition)
GYTFTSYWLH

SEQ ID NO: 43 - protein, 17 aa, synthetic construct
  source 1..17: note = Anti-c-MET-CDR-H2 (Kabat definition)
MIDPSNSDTRFNPNFKD

SEQ ID NO: 44 - protein, 6 aa, synthetic construct
  source 1..6: note = Anti-c-MET-CDR-H2 (Chothia definition)
DPSNSD

SEQ ID NO: 45 - protein, 8 aa, synthetic construct
  source 1..8: note = Anti-c-MET-CDR-H2 (IMGT definition)
IDPSNSDT

SEQ ID NO: 46 - protein, 10 aa, synthetic construct
  source 1..10: note = Anti-c-MET-CDR-H2 (Abm definition)
MIDPSNSDTR

SEQ ID NO: 47 - protein, 10 aa, synthetic construct
  source 1..10: note = Anti-c-MET-CDR-H3 (Kabat/Chothia/Abm definition)
YRSYVTPLDY

SEQ ID NO: 48 - protein, 12 aa, synthetic construct
  source 1..12: note = Anti-c-MET-CDR-H3 (IMGT definition)
ATYRSYVTPLDY

SEQ ID NO: 49 - protein, 217 aa, synthetic construct
  source 1..217: note = Fc (DELM:KIH) (knob) for BsAb 07B and 10B [Fc:D356E:L358M:KIH-knob:S354C:T366W (Fc Eu numbering scheme)]
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 217 aa, synthetic construct
  source 1..217: note = Fc (DELM:KIH) (hole) for BsAb 07B and 10B [Fc:D356E:L358M:KIH-hole (Y349C: T366S:L368A:Y407V Fc Eu numbering scheme)]
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 217 aa, synthetic construct
  source 1..217: note = Fc (LALAPA:KIH (knob) for BsAb 38B, 41B, 49B, 55B and 56B [Fc:L234A:L235A:P329A: KIH-knob:S354C:T366 W (Fc Eu numbering scheme)]
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 217 aa, synthetic construct
  source 1..217: note = Fc (LALAPA:KIH (hole) for BsAb 38B, 41B, 49B, 55B and 56B [Fc:(L234A:L235A:P329A):KIH-hole (Y349C: T366S:L368A:Y407V Fc Eu numbering scheme)]
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 53 - protein, 107 aa, synthetic construct [sequence omitted]

SEQ ID NO: 54 - protein, 103 aa, synthetic construct
  source 1..103: note = CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 55 - protein, 15 aa, synthetic construct
  source 1..15: note = Peptide Linker-1 (GGGGS)3
GGGGSGGGGSGGGGS

SEQ ID NO: 56 - protein, 40 aa, synthetic construct
  source 1..40: note = Peptide Linker-2 (GGGGS)8
GGGGSGGGGSGGGGSGGGGSGGGGSGGGGSGGGGSGGGGS

SEQ ID NO: 57 - protein, 5 aa, synthetic construct
  source 1..5: note = Peptide Linker
GGGGS

SEQ ID NO: 58 - protein, 20 aa, synthetic construct
  source 1..20: note = Peptide linker (GGGGS)4
GGGGSGGGGSGGGGSGGGGS

SEQ ID NO: 59 - protein, 113 aa, synthetic construct
  source 1..113: note = Anti-c-MET-VL (Q106C)
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGCGTKVEIK

SEQ ID NO: 60 - protein, 119 aa, synthetic construct
  source 1..119: note = Anti-c-MET-VH (G44C)
EVQLVESGGGLVQPGGSLRLSCAASGYTFTSYWLHWVRQAPGKCLEWVGMIDPSNSDTRFNPNFKDRFTISADTSKNTAYLQMNSLRAEDTAVYYCATYRSYVTPLDYWGQGTLVTVSS

SEQ ID NO: 61 - protein, 4 aa, synthetic construct
  source 1..4: note = Peptide linker
GGGG

SEQ ID NO: 62 - protein, 10 aa, synthetic construct
  source 1..10: note = Heavy chain CH1 hinge region
DKTHTCPPCP

SEQ ID NO: 63 - protein, 107 aa, synthetic construct
  source 1..107: note = CL for BsAb 55B
ASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 330 aa, synthetic construct
  source 1..330: note = Mutated human IgG1 heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 10 aa, synthetic construct
  source 1..10: note = Peptide linker (GGGGS)2
GGGGSGGGGS

SEQ ID NO: 66 - protein, 25 aa, synthetic construct
  source 1..25: note = Peptide linker (GGGGS)5
GGGGSGGGGSGGGGSGGGGSGGGGS

SEQ ID NO: 67 - protein, 30 aa, synthetic construct
  source 1..30: note = Peptide linker (GGGGS)6
GGGGSGGGGSGGGGSGGGGSGGGGSGGGGS

SEQ ID NO: 68 - protein, 35 aa, synthetic construct
  source 1..35: note = Peptide linker (GGGGS)7
GGGGSGGGGSGGGGSGGGGSGGGGSGGGGSGGGGS

SEQ ID NO: 69 - protein, 45 aa, synthetic construct
  source 1..45: note = Peptide linker (GGGGS)9
GGGGSGGGGSGGGGSGGGGSGGGGSGGGGSGGGGSGGGGSGGGGS

SEQ ID NO: 70 - protein, 50 aa, synthetic construct
  source 1..50: note = Peptide linker (GGGGS)10
GGGGSGGGGSGGGGSGGGGSGGGGSGGGGSGGGGSGGGGSGGGGSGGGGS

SEQ ID NO: 71 - protein, 4 aa, synthetic construct
  source 1..4: note = Gly-Gly-Phe-Gly peptide 1
GGFG

SEQ ID NO: 72 - protein, 4 aa, synthetic construct
  source 1..4: note = Gly-Gly-Val-Ala peptide 2
GGVA

SEQ ID NO: 73 - protein, 4 aa, synthetic construct
  source 1..4: note = Gly-Phe-Leu-Gly peptide 3
GFLG

SEQ ID NO: 74 - protein, 4 aa, synthetic construct
  source 1..4: note = Glu-Ala-Ala-Ala peptide 4
EAAA

SEQ ID NO: 75 - protein, 5 aa, synthetic construct
  source 1..5: note = Gly-Gly-Gly-Gly-Gly peptide 5
GGGGG